(19) United States
(12) Patent Application Publication
(10) Pub. No.: US 2024/0384358 A1
(43) Pub. Date: Nov. 21, 2024

(54) PROFILING HEMATOLOGICAL NEOPLASMS

(71) Applicant: Roswell Park Cancer Institute Corporation, Buffalo, NY (US)

(72) Inventors: Carl Morrison, Buffalo, NY (US); Sean Glenn, Buffalo, NY (US); Manuel Glynias, Buffalo, NY (US)

(21) Appl. No.: 18/833,786

(22) PCT Filed: Jan. 27, 2023
(86) PCT No.: PCT/US2023/011770

§ 371 Date: Jul. 26, 2024

Related U.S. Application Data
(60) Provisional application No. 63/304,391, filed on Jan. 28, 2022.
(60) Provisional application No. 63/428,898, filed on Nov. 30, 2022.

(51) Int. Cl. C12Q 1/6886

(52) U.S. Cl. CPC C12Q 1/6886; C12Q 2600/156; C12Q 2600/158

(57) ABSTRACT

The present disclosure relates to dual DNA-based and RNA-based next generation sequencing assays, comprising a DNA component comprising an oligonucleotide and a detection probe targeting DNA isolated from the hematological neoplasm and an RNA component comprising oligonucleotide and a detection probe targeting RNA isolated from the hematological neoplasm, as well as the treatment of hematological neoplasms.

### RELATED APPLICATION

This PCT application claims priority to, and the benefit of, U.S. Provisional Patent Application No. 63/304,391, filed Jan. 28, 2022 and U.S. Provisional Patent Application No. 63/428,898 filed Nov. 30, 2022, which are incorporated by reference herein in its entirety.

### FIELD

The present disclosure relates next generation sequencing assays and methods for identifying or profiling hematological neoplasms.

### BACKGROUND

High-throughput sequencing technology is an advanced detection method compared to conventional one generation sequencing methods. With high-throughput sequencing, also known as next-generation sequencing (NGS) or deep sequencing, there is capacity to detect and sequence hundreds of thousands or millions of nucleic acid molecules at one time allowing for thorough analyses of transcriptomes and genomes in one subject. Specifically, NGS detection methods can detect numerous genes to meet the high demands of clinical detection, and can not only detect known mutation sites, but also unknown mutation sites. Further, NGS can also detect various types of mutations and gene variants in various types of clinical samples, including, but not limited to full blood, bone marrow, tissue, serum, or isolated DNA or RNA. NGS can also identify and profile various forms of cancer, including hematological neoplasms and other blood related cancers.

Hematological neoplasms are cancers that begin in blood-forming tissue, such as the bone marrow or immune system cells, and impact the normal production and function of blood cells. Genomic and transcriptomic characterization is essential for the diagnosis, risk assessment, therapeutic strategies, disease monitoring, disease progression, and treatment resistance of hematological neoplasms. However, a complete panel of genes and variants thereof, and an assay for profiling these genes are lacking in the clinical setting. Development of a NGS assay for hematological neoplasms would allow for in depth genomic profiling for the necessary diagnosis and disease management in patients affected by hematological neoplasms.

Given the limitations described above, there is a need for a NGS assay for identifying and profiling hematological neoplasms. The assays and methods disclosed herein address these needs.

### SUMMARY

The present disclosure provides a next generation sequencing assay for identifying or profiling hematological neoplasms.

In one aspect disclosed herein are dual DNA-based and RNA-based next generation sequencing (NGS) assays for detecting a hematological neoplasm comprising a DNA component comprising an oligonucleotide and a detection probe targeting DNA isolated from the hematological neoplasm and an RNA component comprising oligonucleotide and a detection probe targeting RNA isolated from the hematological neoplasm, and wherein the hematopoietic neoplasm is selected from acute myeloid leukemia (AML), myelodysplastic syndrome (MDS), myeloproliferative neoplasm (MPN), non-Hodgkin lymphoma (NHL), chronic lymphocytic leukemia (CLL), and multiple myeloma (MM). In some aspects, the nucleic acid sequence targeted by the assay encodes BRAF, EZH2, KIT, MYD88, TP53, TET2, or a gene expressed in AML, MDS, MPN, NHL, CLL, or MM. For example, the nucleic acid sequence targeted by the DNA component can encode a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCL, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF21, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3KL, MAP3K14, MCL1, MECOM, MED12, MEF2B, MET, MGA, MLH1, MPEG1, MPL, MSH2, MSH6, MTOR, MYB, MYC, MYD88, NBN, NF1, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2. In one aspect the DNA component of the dual DNA-based and RNA-based NGS assay detects at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, or all 345 of said heme-related genes.

Also disclosed herein are dual DNA-based and RNA-based NGS assays of any preceding aspect, wherein the DNA component of the NGS assay detects a variant selected from a group consisting of a single nucleotide variant, an insertion deletion (indel), a chimeric fusion, a structural variant, a copy number variant, and a chromosomal abnormality.

In one aspect, disclosed herein are dual DNA-based and RNA-based NGS assays of any preceding aspect, wherein the DNA component of the NGS assay simultaneously detects more than one variant within the hematological neoplasm.

Also disclosed herein are dual DNA-based and RNA-based NGS assays of any preceding aspect, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2.

In one aspect, disclosed herein are dual DNA-based and RNA-based NGS assays of any preceding aspect, wherein the gene expressed in MDS is selected from ASXL1, ATM, BCOR, CALR, CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

Also disclosed herein are dual DNA-based and RNA-based NGS assays of any preceding aspect, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

In one aspect, disclosed herein are dual DNA-based and RNA-based NGS assays of any preceding aspect, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

Also disclosed herein are dual DNA-based and RNA-based NGS assay of any preceding aspect, wherein the gene expressed in CLL is selected from ATM, BCL2, BTK, NOTCH1, PLCG2, RB1, SF3B1, and TP53.

In one aspect, disclosed herein are dual DNA-based and RNA-based NGS assays of any preceding aspect, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

Also disclosed herein are dual DNA-based and RNA-based NGS assays of any preceding aspect, wherein the DNA component of the NGS assay detects one or more single nucleotide variations listed in Table 29.

Also disclosed herein are dual DNA-based and RNA-based NGS assays of any preceding aspect, wherein the DNA component of the NGS assay simultaneously detects a gene variant and the sytogenic alternation selected from the group consisting of an EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, an odd number of chromosomes, and trisomy 21.

In one aspect, disclosed herein are dual DNA-based and RNA-based NGS assays of any preceding aspect, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, an odd number of chromosomes, or trisomy 21.

Also disclosed herein are dual DNA-based and RNA-based NGS assays of any preceding aspect, wherein the RNA component of the NGS assay detects a chimeric gene fusion without prior knowledge of the fusion partners or the breakpoint translocation.

In one aspect, disclosed herein are dual DNA-based and RNA-based NGS assays of any preceding aspect, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CIITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCH1, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TAL1, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUXL, SEPTIN9, TP63, SPI1, RNF213, TCL1A, ELL, DDX3X, or HNRNPC

Also disclosed herein are dual DNA-based and RNA-based NGS assays of any preceding aspect, wherein the chimeric fusion comprises CCND1-IGH, FGFR3-IGH, BCL6-IGH, BCL2-IGH, MYC-IGH, MALT1-IGH, AP12-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are dual DNA-based and RNA-based NGS assays of any preceding aspect, wherein the chimeric fusion in mantel cell lymphoma (MCL) comprises CCND1-IGH.

Also disclosed herein are dual DNA-based and RNA-based NGS assays of any preceding aspect, wherein the chimeric fusion in MM comprises CCND1-IGH or FGFR3-IGH.

In one aspect, disclosed herein are dual DNA-based and RNA-based NGS assays of any preceding aspect, wherein the chimeric fusion in diffuse large B-cell lymphoma (DLBCL) comprises BCL6-IGH or BCL2-IGH.

Also disclosed herein are dual DNA-based and RNA-based NGS assays of any preceding aspect, wherein the chimeric fusion in follicular lymphoma (FL) comprises BCL6-IGH, BCL2-IGH, or MYC-IGH.

In one aspect, disclosed herein are dual DNA-based and RNA-based NGS assays of any preceding aspect, w % herein the chimeric fusion in Burkitt lymphoma comprises MYC-IGH.

Also disclosed herein are dual DNA-based and RNA-based NGS assays of any preceding aspect, wherein the chimeric fusion in mucosal-associated lymphoma (MALT) comprises MALT1-IGH or API2-MALT1.

In one aspect, disclosed herein are dual DNA-based and RNA-based NGS assays of any preceding aspect, wherein the chimeric fusion in B-acute lymphocytic leukemia (B-ALL) comprises ETV6-RUNX1(TEL-AML1), TCF3-PBX1 (E2A-PBX), ABL1-BCR, or AF1-MLL.

Also disclosed herein are dual DNA-based and RNA-based NGS assays of any preceding aspect, wherein the chimeric fusion in chronic myelogenous leukemia (CML) comprises ABL1-BCR.

In one aspect, disclosed herein are dual DNA-based and RNA-based NGS assays of any preceding aspect, wherein the chimeric fusions detected in AML or an AML related therapy comprises ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, or RUNX1-ZFPM2.

Also disclosed herein are dual DNA-based and RNA-based NGS assays of any preceding aspect, wherein the chimeric fusions detected in MDS comprise CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are dual DNA-based and RNA-based NGS assays of any preceding aspect, wherein the chimeric fusions detected in MPN comprise KANK1-PDGFRB, ETV6-LYN, PCM1-JAK2, PDE4DIP-PDGFRB, or GOLGIB1-PDGFRB.

Also disclosed herein are dual DNA-based and RNA-based NGS assays of any preceding aspect, wherein the chimeric fusion detected in NHL comprises CCND1-IGH, FGFR3-IGH, BCL6-IGH, BCL2-IGH, MYC-IGH, MALT1-IGH, or API2-MALT1.

In one aspect, disclosed herein are dual DNA-based and RNA-based NGS assays of any preceding aspect, wherein the sequencing assay further comprises a gene library construction reagent.

Also disclosed herein are dual DNA-based and RNA-based NGS assays of any preceding aspect, wherein the sequencing assay further comprises a gene amplification reagent.

In one aspect, disclosed herein are dual DNA-based and RNA-based NGS assays of any preceding aspect, wherein the sequencing assay further comprises a verification sample comprising a nucleic acid standard composition encoding one or more genes selected from ABL1, ABL2, ACTB, AFF1, AHI1, ALK, ASXL1, ASXL2, ATF7IP, ATM, BCL11B, BCL2, BCL6, BCOR, BCR, BIRC3, BMI1, BTK, CALR, CBFB, CBFA2T3, CBL, CCND1, CCND3, CD28, CDK6, CDKN2C, CEP43, CEBPA, CHD1, CHEK2, CHIC2, CIITA, CREBBP, CRLF2, CSF1R, CUX1, CXCR4, DAZAP1, DDX6, DDX10, DDX3X, DDX41, DEK, DIS3, DNMT3A, DSCAM, DUSP22, EBF1, EGR1, EIF4A1, ELL, EP300, EPOR, ERG, ETS2, ETV6, FGFR1, FGFR3, FLT3, FOXP1, FOXR1, FUS, GATA2, GLIS2, H2AX, HNRNPC, HOXA9, HOXA11, IDH1, IDH2, IGH, IGK, IGL, IKZF1, IKZF2, IKZF3, IRF4, JAK2, KANSL1, KAT6A, KDM5A, KLF2, KMT2A, KMT2C, KRAS, LMO1, LMO2, LYN, MALT1, MAP2K1, MECOM, MEK, MLF1, MLLT4, MLLT10, MKL1, MPL, MN1, MPO, MRTFA, MSI2, MTCP1, MYB, MYC, MYH9, MYH11, NF1, NFIA, NFE2, NFKB2, NIPBL, NOTCH1, NPM1, NRAS, NSD1, NTRK3, NUP214, NUP98, NUTM1, P2RY8, PAG1, PAX5, PBX1, PDCD1LG2, PDGFRA, PDGFRB, PHF6, PICALM, PLCG2, PML, PPM1D, PRDM16, PRRC2B, PTK2B, RARA, RARG, RB1, RBM15, RET, ROS1, RNF213, RUNX1, RUNX1T1, SEC31A, SETBP1, SEMA6A, SEPTIN9, SETD2, SF3B1, SH2B3, SPI1, SRSF2, SSBP2, STAG2, STAT3, STAT6, STIL, SYK, TAL1, TBL1XR1, TCF3, TCL1A, TFG, TP63, TP73, TRAF2, TPM3, TPM4, TRA, TRB, TRD, TYK2, U2AF1, UBA2, VAV1, WT1, XPO1, YPEL5, ZCCHC7, ZFP36L2, ZNF362, ZNF384, and ZRSR2.

Also disclosed herein are dual DNA-based and RNA-based NGS assays of any preceding aspect, wherein the detection probe emits a light or a color signal w % ben the oligonucleotide interacts with the nucleic acid sequence.

In one aspect, disclosed herein are methods of identifying or profiling a hematological neoplasm in a subject in need thereof, the method comprising a) obtaining a tissue sample from the subject, b) performing the dual DNA-based and RNA-based NGS assay of any preceding aspect, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). For example, disclosed herein are methods of identifying or profiling a hematological neoplasm in a subject in need thereof, the method comprising a) obtaining a tissue sample from the subject; b) performing dual DNA-based and RNA-based next generation sequencing (NGS) assay on the tissue sample using an oligonucleotide and a detection probe targeting DNA isolated from the tissue sample and an oligonucleotide and a detection probe targeting RNA isolated from the tissue sample, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM).

In one aspect, disclosed herein are methods of identifying or profiling a hematological neoplasm of any preceding aspect, wherein the nucleic acid sequence targeted by the DNA component encodes a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF2I, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NFL, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2. In some aspects, the DNA component of the dual DNA-based and RNA-based NGS assay detects at least 1, 2, 3, 4, 5, 6.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, or all 345 of said heme-related genes.

Also disclosed herein are methods of identifying or profiling a hematological neoplasm of any preceding aspect, wherein the NGS assay detects a variant selected from a group consisting of a single nucleotide variant, an insertion deletion (indel), a chimeric fusion, a structural variant, a copy number variant, and a chromosomal abnormality.

In one aspect, disclosed herein are methods of identifying or profiling a hematological neoplasm of any preceding aspect, wherein the nucleic acid sequence encodes BRAF, EZH2, KIT, MYD88, TP53, TET2, or a gene expressed in AML, MDS, MPN, NHL, CLL, or MM.

In one aspect, disclosed herein are methods of identifying or profiling a hematological neoplasm of any preceding aspect, wherein the NGS assay simultaneously detects more than one variants within the hematological neoplasm.

Also disclosed herein are methods of identifying or profiling a hematological neoplasm of any preceding aspect, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2.

In one aspect, disclosed herein are methods of identifying or profiling a hematological neoplasm of any preceding aspect, wherein the gene expressed in MDS is selected from ASXL1. ATM, BCOR, CALR, CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

Also disclosed herein are methods of identifying or profiling a hematological neoplasm of any preceding aspect, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

In one aspect, disclosed herein are methods of identifying or profiling a hematological neoplasm of any preceding aspect, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

Also disclosed herein are methods of identifying or profiling a hematological neoplasm of any preceding aspect, wherein the gene expressed in CLL is selected from ATM, BCL2, BTK, NOTCH1. PLCG2, RB1, SF3B1, and TP53.

In one aspect, disclosed herein are methods of identifying or profiling a hematological neoplasm of any preceding aspect, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

Also disclosed herein are methods of identifying or profiling a hematological neoplasm of any preceding aspect, wherein the DNA component of the NGS assay detects one or more single nucleotide variations listed in Table 29.

Also disclosed herein are methods of identifying or profiling a hematological neoplasm of any preceding aspect, wherein the DNA component of the NGS assay detects a gene variant and the chromosomal abnormality selected from EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, and an odd number of chromosomes.

In one aspect, disclosed herein are methods of identifying or profiling a hematological neoplasm of any preceding aspect, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, or an odd number of chromosomes.

Also disclosed herein are methods of identifying or profiling a hematological neoplasm of any preceding aspect, wherein the RNA component of the NGS assay detects a chimeric gene fusion without prior knowledge of the fusion partners or the breakpoint translocation.

In one aspect, disclosed herein are methods of identifying or profiling a hematological neoplasm of any preceding aspect, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CIITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCH1, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TAL1, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUX1, SEPTIN9, TP63, SPI1, RNF213, TCL1A, ELL, DDX3X, or HNRNPC

Also disclosed herein are methods of identifying or profiling a hematological neoplasm of any preceding aspect, wherein the chimeric fusion comprises CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of identifying or profiling a hematological neoplasm of any preceding aspect, wherein the chimeric fusion in mantel cell lymphoma (MCL) comprises CCND1-IGH.

Also disclosed herein are methods of identifying or profiling a hematological neoplasm of any preceding aspect, wherein the chimeric fusion in MM comprises CCND1-IGH or FGFR3-IGH.

In one aspect, disclosed herein are methods of identifying or profiling a hematological neoplasm of any preceding aspect, wherein the chimeric fusion in diffuse large B-cell lymphoma (DLBCL) comprises BCL6-IGH or BCL2-IGH.

Also disclosed herein are methods of identifying or profiling a hematological neoplasm of any preceding aspect, wherein the chimeric fusion in follicular lymphoma (FL) comprises BCL6-IGH, BCL2-IGH, or MYC-IGH.

In one aspect, disclosed herein are methods of identifying or profiling a hematological neoplasm of any preceding aspect, wherein the chimeric fusion in Burkitt lymphoma comprises MYC-IGH.

Also disclosed herein are methods of identifying or profiling a hematological neoplasm of any preceding aspect, wherein the chimeric fusion in mucosal-associated lymphoma (MALT) comprises MALT1-IGH or API2-MALT1.

In one aspect, disclosed herein are methods of identifying or profiling a hematological neoplasm of any preceding aspect, wherein the chimeric fusion in B-acute lymphocytic leukemia (B-ALL) comprises ETV6-RUNX1(TEL-AML1), TCF3-PBX1 (E2A-PBX), ABL1-BCR, or AF1-MLL.

Also disclosed herein are methods of identifying or profiling a hematological neoplasm of any preceding aspect, wherein the chimeric fusion in chronic myelogenous leukemia (CML) comprises ABL1-BCR.

In one aspect, disclosed herein are methods of identifying or profiling a hematological neoplasm of any preceding aspect, wherein the chimeric fusions detected in AML or an AML related therapy comprises ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, or RUNX1-ZFPM2.

Also disclosed herein are methods of identifying or profiling a hematological neoplasm of any preceding aspect, wherein the chimeric fusions detected in MDS comprise CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of identifying or profiling a hematological neoplasm of any preceding aspect, wherein the chimeric fusions detected in MPN comprise KANK1-PDGFRB, ETV6-LYN, PCM1-JAK2, PDE4DIP-PDGFRB, or GOLGIB1-PDGFRB.

Also disclosed herein are methods of identifying or profiling a hematological neoplasm of any preceding aspect, wherein the chimeric fusion detected in NHL comprises CCND1-IGH, FGFR3-IGH, BCL6-IGH, BCL2-IGH, MYC-IGH, MALT1-IGH, or AP12-MALT1.

In one aspect, disclosed herein are methods of identifying or profiling a hematological neoplasm of any preceding aspect, wherein the detection probe emits a light or a color signal when the oligonucleotide interacts with the nucleic acid sequence.

Also disclosed herein are methods of identifying or profiling a hematological neoplasm of any preceding aspect, wherein the tissue sample is a blood, bone marrow, serum, or fluid sample.

In one aspect, disclosed herein are methods of identifying or profiling a hematological neoplasm of any preceding aspect, wherein the method further comprises administering to the subject a therapeutically effective amount of an anti-cancer agent appropriate for the treatment of the hematological neoplasm identified.

Also disclosed herein are methods of treating, inhibiting, reducing, decreasing, ameliorating, and/or preventing a hematological neoplasm in a subject comprising identifying or profiling a hematological neoplasm by the methods of any preceding aspect and/or using the dual DNA-based and RNA-based assay of any preceding aspect and administering to the subject a therapeutically effective amount of an anti-cancer agent appropriate for the treatment of the hematological neoplasm identified. For example, disclosed herein are methods of treating, inhibiting, reducing, decreasing, ameliorating, and/or preventing a hematological neoplasm in a subject in need thereof, the method comprising a) obtaining a tissue sample from the subject; b) performing dual DNA-based and RNA-based next generation sequencing (NGS) assay on the tissue sample using an oligonucleotide and a detection probe targeting DNA isolated from the tissue sample and an oligonucleotide and a detection probe targeting RNA isolated from the tissue sample, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). In some aspects, the nucleic acid sequence detected by the assay encodes BRAF. EZH2, KIT, MYD88, TP53, TET2, or a gene expressed in AML, MDS, MPN, NHL, CLL, or MM.

In one aspect, disclosed herein are methods of treating, inhibiting, reducing, decreasing, ameliorating, and/or preventing a hematological neoplasm of any preceding aspect, wherein the nucleic acid sequence targeted by the DNA component encodes a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF21, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NF1, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2. In some aspects, the DNA component of the dual DNA-based and RNA-based NGS assay detects at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, or all 345 of said heme-related genes.

Also disclosed herein are methods of treating, inhibiting, reducing, decreasing, ameliorating, and/or preventing a hematological neoplasm of any preceding aspect, wherein the NGS assay detects a variant selected from a group consisting of a single nucleotide variant, an insertion deletion (indel), a chimeric fusion, a structural variant, a copy number variant, and a chromosomal abnormality.

In one aspect, disclosed herein are methods of treating, inhibiting, reducing, decreasing, ameliorating, and/or preventing a hematological neoplasm of any preceding aspect, wherein the nucleic acid sequence encodes BRAF, EZH2, KIT, MYD88, TP53, TET2, or a gene expressed in AML, MDS, MPN, NHL, CLL, or MM.

In one aspect, disclosed herein are methods of treating, inhibiting, reducing, decreasing, ameliorating, and/or preventing a hematological neoplasm of any preceding aspect, wherein the NGS assay simultaneously detects more than one variants within the hematological neoplasm.

Also disclosed herein are methods of treating, inhibiting, reducing, decreasing, ameliorating, and/or preventing a hematological neoplasm of any preceding aspect, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2.

In one aspect, disclosed herein are methods of treating, inhibiting, reducing, decreasing, ameliorating, and/or preventing a hematological neoplasm of any preceding aspect, wherein the gene expressed in MDS is selected from ASXL1, ATM, BCOR, CALR, CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

Also disclosed herein are methods of treating, inhibiting, reducing, decreasing, ameliorating, and/or preventing a hematological neoplasm of any preceding aspect, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

In one aspect, disclosed herein are methods of treating, inhibiting, reducing, decreasing, ameliorating, and/or preventing a hematological neoplasm of any preceding aspect, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

Also disclosed herein are methods of treating, inhibiting, reducing, decreasing, ameliorating, and/or preventing a hematological neoplasm of any preceding aspect, wherein the gene expressed in CLL is selected from ATM, BCL2, BTK, NOTCH1, PLCG2, RB1, SF3B1, and TP53.

In one aspect, disclosed herein are methods of treating, inhibiting, reducing, decreasing, ameliorating, and/or preventing a hematological neoplasm of any preceding aspect, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

Also disclosed herein are methods of treating, inhibiting, reducing, decreasing, ameliorating, and/or preventing a hematological neoplasm of any preceding aspect, wherein the DNA component of the NGS assay detects one or more single nucleotide variations listed in Table 29.

Also disclosed herein are methods of treating, inhibiting, reducing, decreasing, ameliorating, and/or preventing a hematological neoplasm of any preceding aspect, wherein the DNA component of the NGS assay detects a gene variant and the chromosomal abnormality selected from EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11 q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, and an odd number of chromosomes.

In one aspect, disclosed herein are methods of treating, inhibiting, reducing, decreasing, ameliorating, and/or preventing a hematological neoplasm of any preceding aspect, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 1 q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, or an odd number of chromosomes.

Also disclosed herein are methods of treating, inhibiting, reducing, decreasing, ameliorating, and/or preventing a hematological neoplasm of any preceding aspect, wherein the RNA component of the NGS assay detects a chimeric gene fusion without prior knowledge of the fusion partners or the breakpoint translocation.

In one aspect, disclosed herein are methods of treating, inhibiting, reducing, decreasing, ameliorating, and/or preventing a hematological neoplasm of any preceding aspect, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CIITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCH1, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TAL1, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUX1, SEPTIN9, TP63, SPI1, RNF213, TCL1A, ELL, DDX3X, or HNRNPC

Also disclosed herein are methods of treating, inhibiting, reducing, decreasing, ameliorating, and/or preventing a hematological neoplasm of any preceding aspect, wherein the chimeric fusion comprises CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of treating, inhibiting, reducing, decreasing, ameliorating, and/or preventing a hematological neoplasm of any preceding aspect, wherein the chimeric fusion in mantel cell lymphoma (MCL) comprises CCND1-IGH.

Also disclosed herein are methods of treating, inhibiting, reducing, decreasing, ameliorating, and/or preventing a hematological neoplasm of any preceding aspect, wherein the chimeric fusion in MM comprises CCND1-IGH or FGFR3-IGH.

In one aspect, disclosed herein are methods of treating, inhibiting, reducing, decreasing, ameliorating, and/or preventing a hematological neoplasm of any preceding aspect, wherein the chimeric fusion in diffuse large B-cell lymphoma (DLBCL) comprises BCL6-IGH or BCL2-IGH.

Also disclosed herein are methods of treating, inhibiting, reducing, decreasing, ameliorating, and/or preventing a hematological neoplasm of any preceding aspect, wherein the chimeric fusion in follicular lymphoma (FL) comprises BCL6-IGH, BCL2-IGH, or MYC-IGH.

In one aspect, disclosed herein are methods of treating, inhibiting, reducing, decreasing, ameliorating, and/or preventing a hematological neoplasm of any preceding aspect, wherein the chimeric fusion in Burkitt lymphoma comprises MYC-IGH.

Also disclosed herein are methods of treating, inhibiting, reducing, decreasing, ameliorating, and/or preventing a hematological neoplasm of any preceding aspect, wherein the chimeric fusion in mucosal-associated lymphoma (MALT) comprises MALT1-IGH or API2-MALT1.

In one aspect, disclosed herein are methods of treating, inhibiting, reducing, decreasing, ameliorating, and/or preventing a hematological neoplasm of any preceding aspect, wherein the chimeric fusion in B-acute lymphocytic leukemia (B-ALL) comprises ETV6-RUNX1(TEL-AML1), TCF3-PBX1 (E2A-PBX), ABL1-BCR, or AF1-MLL.

Also disclosed herein are methods of treating, inhibiting, reducing, decreasing, ameliorating, and/or preventing a hematological neoplasm of any preceding aspect, wherein the chimeric fusion in chronic myelogenous leukemia (CML) comprises ABL1-BCR.

In one aspect, disclosed herein are methods of treating, inhibiting, reducing, decreasing, ameliorating, and/or preventing a hematological neoplasm of any preceding aspect, wherein the chimeric fusions detected in AML or an AML related therapy comprises ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, or RUNX1-ZFPM2.

Also disclosed herein are methods of treating, inhibiting, reducing, decreasing, ameliorating, and/or preventing a hematological neoplasm of any preceding aspect, wherein the chimeric fusions detected in MDS comprise CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of treating, inhibiting, reducing, decreasing, ameliorating, and/or preventing a hematological neoplasm of any preceding aspect, wherein the chimeric fusions detected in MPN comprise KANK1-PDGFRB, ETV6-LYN, PCM1-JAK2, PDE4DIP-PDGFRB, or GOLGIB1-PDGFRB.

Also disclosed herein are methods of treating, inhibiting, reducing, decreasing, ameliorating, and/or preventing a hematological neoplasm of any preceding aspect, wherein the chimeric fusion detected in NHL comprises CCND1-IGH, FGFR3-IGH, BCL6-IGH, BCL2-IGH, MYC-IGH, MALT1-IGH, or API2-MALT1.

In one aspect, disclosed herein are methods of treating, inhibiting, reducing, decreasing, ameliorating, and/or preventing a hematological neoplasm of any preceding aspect, wherein the detection probe emits a light or a color signal when the oligonucleotide interacts with the nucleic acid sequence.

Also disclosed herein are methods of treating, inhibiting, reducing, decreasing, ameliorating, and/or preventing a hematological neoplasm of any preceding aspect, wherein the tissue sample is a blood, bone marrow, serum, or fluid sample.

### BRIEF DESCRIPTION OF FIGURES

The accompanying figures, which are incorporated in and constitute a part of this specification, illustrate several aspects described below.

FIG. 1 shows the clinical value of the hematological neoplasmic gene panel of the present disclosure relative to other heme panels.

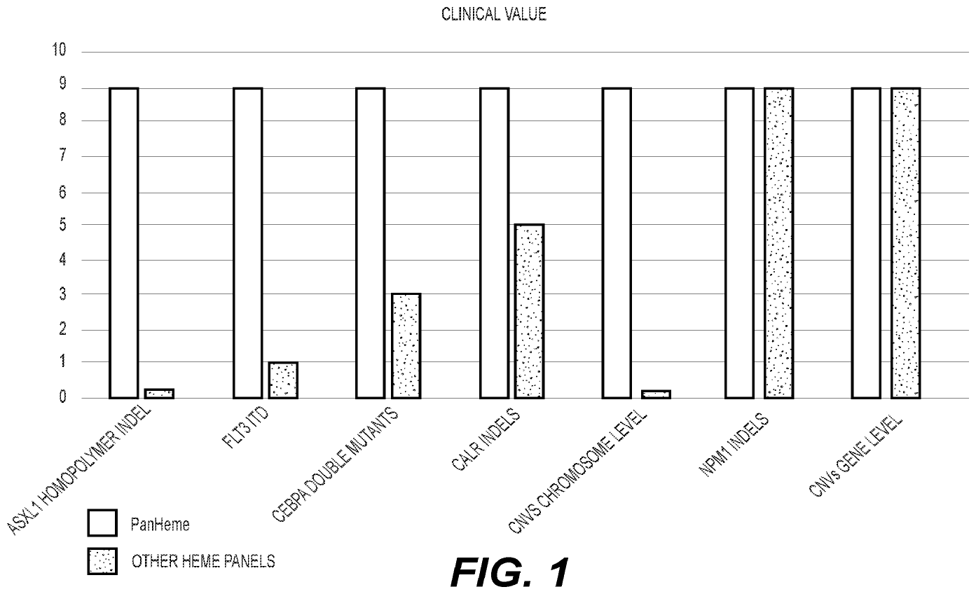

FIG. 2 shows the workflow diagram of a next generation sequencing assay for hematological neoplasms.

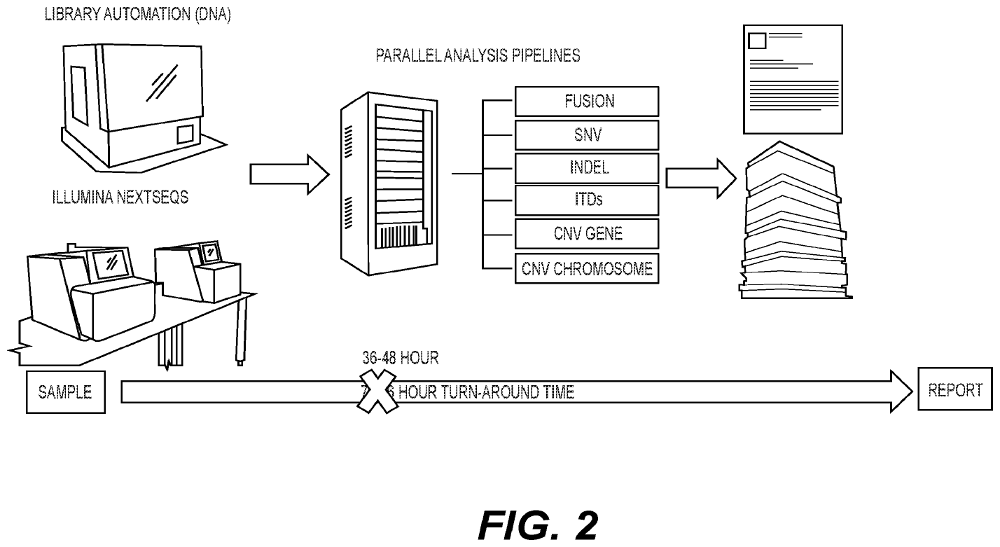

FIGS. 3A, 3B, and 3C show the validation and testing results of the next generation sequencing assay for hematological neoplasms.

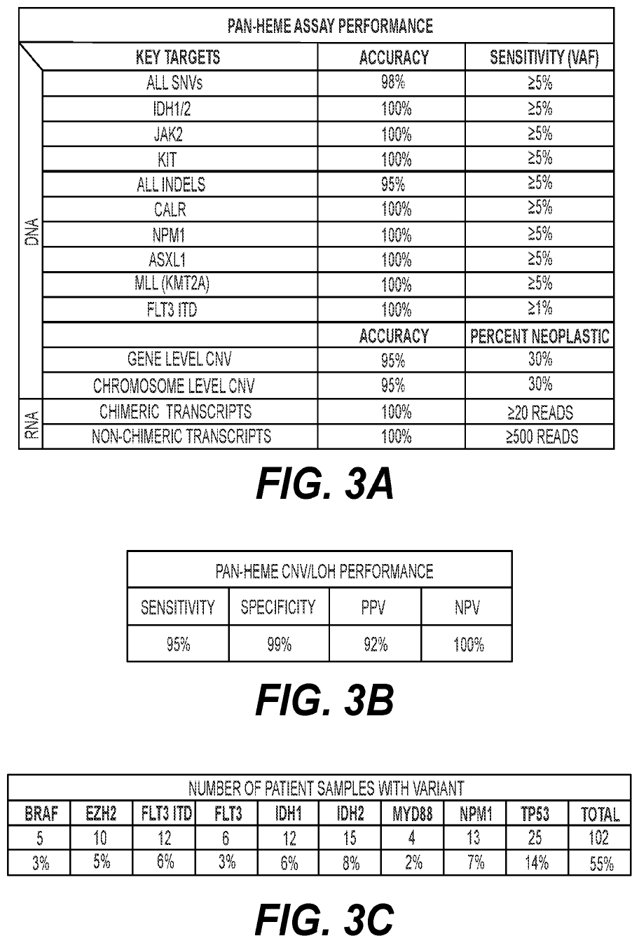

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

The term “detect” or “detecting” refers to an output signal released for the purpose of sensing of physical phenomenon. An event or change in environment is sensed and signal output released in the form of light, heat, or a color change (i.e.: color change from red to blue, white to black, or vice versa).

The term “cancer” is used to address any neoplastic disease. Specifically, it is used here to describe hematologic malignancies, including lymphomas, leukemias and myeloma.

The term “kit” describes a wide variety of bags, containers, carrying cases, and other portable enclosures which may be used to carry and store solid substances, liquid substances, and other accessories necessary to perform a next generation sequencing, deep sequencing, or high-throughput sequencing/screening assays. Such kits and their contents along with any applicable procedures may be used to provide access to variations detected in the hematological neoplasms in accordance with the teachings of the present disclosure.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated. A “gene panel” refers to a compilation, list, or grouping of genes for testing of variations, mutations, and changes in one or more genes. “Profiling” refers to a collection of information relating to a specific gene, protein, disease, or disorder. Profiles can describe the risk of disease, preventive care assessments and utilization, and disease prevalence.

An oligonucleotide refers to a synthetic, short, single-stranded nucleic acid sequence (typically 10-30 nucleotides in length) used to target specified DNA or RNA sequences of interest in a wide range of applications, including, but not limited to biotechnology and genetics testing. The length of an oligonucleotide is usually denoted by “-mer”. For example, an oligonucleotide comprising six nucleotides is a hexamer and an oligonucleotide comprising 25 nucleotides is a “25-mer”. Oligonucleotides can be chemically modified to comprise a detection probe including, but not limited to a fluorophore, an enzyme, a protein, or compound. An oligonucleotide can also be a primer often used in a “polymerase chain reaction”. PCR is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally stable polymerase enzyme. Methods for PCR are well known in the art, and taught, for example in “PCR: A PRACTICAL APPROACH” (M. MacPherson et al., IRL Press at Oxford University Press (1991)).

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A, Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

As used herein, a “mutation” refers to changing the structure of a gene, resulting in a variant form that may be transmitted to later generations. A mutation is caused by the alteration of single nucleotides in DNA, or the deletion, insertion, or rearrangement of larger sections of genes. A mutation can lead to the expression of a protein that has been changed physically or functionally leading to lethality, non-lethal dysfunction effects, or no effects.

An “assay standard”, “standard”, or a“verification sample” refers to any molecule, compound, or composition of known quantity (concentration, volume, mass, etc.) used to determine the quantity of an unknown molecule, compound, or composition. A standard is usually used in an assay to quantify a final product of the assay.

“Hybridizing” refers to the process in which two complementary nucleic acid molecules bind together to form a new double stranded molecule. Binding between the two nucleic acid molecules depends on the appropriate base pairing across the two single stranded molecules.

“Fusing” refers to the process in which two different (usually non-complementary) nucleic acid molecules are joined together to form a new double stranded molecule. Fusing is also an induced process, not dependent on sequence base pairing to force generation of new genes.

### Next Generation Sequencing (NGS) Assays

Next-generation sequencing is a massive parallel sequencing technique wherein isolated DNA or RNA samples are amplified, sequenced, and analyzed for complete genomic or transcriptomic overview. This method of sequencing requires smaller sample sizes, less reagents and time, and lower reagent costs while providing whole genome data from a single subject. The present disclosure describes next generation sequencing assays and methods for profiling hematological neoplasms.

In one aspect disclosed herein are dual DNA-based and RNA-based next generation sequencing (NGS) assays for detecting, identifying, and/or profiling a hematological neoplasm comprising a DNA component comprising an oligonucleotide and a detection probe targeting DNA isolated from the hematological neoplasm and an RNA component comprising oligonucleotide and a detection probe targeting RNA isolated from the hematological neoplasm, and wherein the hematopoietic neoplasm is selected from acute myeloid leukemia (AML), myelodysplastic syndrome (MDS), myeloproliferative neoplasm (MPN), non-Hodgkin lymphoma (NHL), chronic lymphocytic leukemia (CLL), and multiple myeloma (MM). In some aspects, the nucleic acid sequence targeted by the DNA component encodes BRAF, EZH2, KIT, MYD88, TP53, TET2, or a gene expressed in AML, MDS, MPN, NHL, CLL, or MM. For example, the nucleic acid sequence targeted by the DNA component can encode a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF2I, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NFL, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2. It is understood and herein contemplated that the DNA component is not limited to the detection of a single nucleic acid, but can detect multiple nucleic acids simultaneously with the use of multiple oligonucleotides and probes. Accordingly, in one aspect the DNA component of the dual DNA-based and RNA-based NGS assay detects at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, or all 345 of said heme-related genes.

As noted above, the DNA-based component of the dual DNA-based and RNA-based NGS assay comprises an oligonucleotide that is complementary to the nucleic acid sequence isolated from the hematological neoplasm. Thus, in some embodiments, the NGS assay is a DNA-based assay comprising hybridizing the oligonucleotide to a DNA sequence. In this respect, the DNA component of the NGS assay detects a variant selected from a group consisting of a single nucleotide variant, an insertion deletion (indel), a chimeric fusion, a structural variant, a copy number variant, and a chromosomal abnormality. Similarly, the RNA-based component of the dual DNA-based and RNA-based NGS assay comprises an oligonucleotide that is complementary or non-complementary to the nucleic acid sequence isolated from the hematological neoplasm. In some embodiments, the NGS assay is an RNA-based assay comprising fusing the oligonucleotide to an RNA sequence. In some embodiments, the oligonucleotides comprises 15, 16, 17, 18, 19, 20, 21, 22, 23, 24, 25, or more nucleotides.

The disclosed dual DNA-based and RNA-based NGS assay detecting, identifying, and/or profiling neoplasms in particular neoplasms that are characterized into one of six classifications of neoplasms: acute myeloid leukemia (AML), myelodysplastic syndrome (MDS), myeloproliferative neoplasm (MPN), non-Hodgkin lymphoma (NHL), chronic lymphocytic leukemia (CLL), and multiple myeloma (MM). It is understood and herein contemplated that w % herein the neoplasms is an AML, the AML includes, but is not limited to acute lymphoblastic leukemia (ALL), Lymphoblastic (diffuse) lymphoma, acute myeloblastic leukemia, chronic lymphoblastic leukemia of B cell type, myeloid leukemia, atypical chronic myeloid leukemia BCR/ABL—negative, myeloid sarcoma, acute promyelocytic leukemia, acute myelomonocytic leukemia, acute myeloid leukemia with 11q23-abnormality, chronic myeloid leukemia (CML), B-acute lymphocytic leukemia (B-ALL), and acute myeloid leukemia with multidimensional dysplasia. Similarly, wherein the neoplasm is classified as an MDS, the MDS includes, but is not limited to refractory anemia, refractory cytopenia with multilineage dysplasia, refractory anemia with ringed sideroblasts, refractory cytopenia with multilineage dysplasia and ringed sideroblasts, and refractory anemia with excess blasts. Likewise, wherein the neoplasm is classified as a MPN, the MPN includes, but is not limited to polycythemia vera, chronic myeloproliferative disease, and essential (hemorrhagic) thrombocythemia. It is further understood that wherein the neoplasm classifies as a NHL, the NHL includes, but is not limited to small cell B cell lymphoma (splenic marginal zone lymphoma), mantle cell lymphoma, multiple myeloma, diffuse large B cell lymphoma, peripheral T cell lymphoma (not classified), Waldenstrom macroglobulinemia (WM), follicular lymphoma, Burkitt lymphoma, mucosal-associated lymphoma, and post-transplant lymphoproliferative disorder (PTLD). Additionally, wherein the neoplasm classifies as a CLL, the CLL includes, but is not limited to chronic lymphocytic leukemia of B cell type, small lymphocytic lymphoma (SLL), prolymphocytic leukemia (PLL), large granular lymphocyte (LGL) leukemia, and hairy cell leukemia (HCL). Lastly, when the neoplasm classifies as a MM, the MM includes, but is not limited to smoldering multiple myeloma (SMM), light chain amyloidosis, and Waldenstrom macroglobulinemia (WM).

In some embodiments, the nucleic acid sequence targeted by the DNA component encodes BRAF, EZH2, KIT, MYD88, TP53, TET2, or a gene expressed in AML, MDS, MPN, NHL, CLL, or MM, such as, for example, ABL1, ABL2, ACTB, AFF1, AHI1, ALK, ASXL1, ASXL2, ATF7IP, ATM, BCL1B, BCL2, BCL6, BCOR, BCR, BIRC3, BMI1, BTK, CALR, CBFB, CBFA2T3, CBL, CCND1, CCND3, CD28, CDK6, CDKN2C, CEP43, CEBPA, CHD1, CHEK2, CHIC2, CIITA, CREBBP, CRLF2, CSF1R, CUX1, CXCR4, DAZAP1, DDX6, DDX10, DDX3X, DDX41, DEK, DIS3, DNMT3A, DSCAM, DUSP22, EBF1, EGR1, EIF4A1, ELL, EP300, EPOR, ERG, ETS2, ETV6, FGFR1, FGFR3, FLT3, FOXP1, FOXR1, FUS, GATA2, GLIS2, H2AX, HNRNPC, HOXA9, HOXA11, IDH1, IDH2, IGH, IGK, IGL, IKZF1, IKZF2, IKZF3, IRF4, JAK2, KANSL1, KAT6A, KDM5A, KLF2, KMT2A, KMT2C, KRAS, LMO1, LMO2, LYN, MALT1, MAP2K1, MECOM, MEK, MLF1, MLLT4, MLLT10, MKL1, MPL, MN1, MPO, MRTFA, MSI2, MTCP1, MYB, MYC, MYH9, MYH11, NF1, NFIA, NFE2, NFKB2, NIPBL, NOTCH1, NPM1, NRAS, NSD1, NTRK3, NUP214, NUP98, NUTM1, P2RY8, PAG1, PAX5, PBX1, PDCD1LG2, PDGFRA, PDGFRB, PHF6, PICALM, PLCG2, PML, PPM1D, PRDM16, PRRC2B, PTK2B, RARA, RARG, RB1, RBM15, RET, ROS1, RNF213, RUNX1, RUNX1T1, SEC31A, SETBP1, SEMA6A, SEPTIN9, SETD2, SF3B1, SH2B3, SPI1, SRSF2, SSBP2, STAG2, STAT3, STAT6, STIL, SYK, TAL1, TBL1XR1, TCF3, TCL1A, TFG, TP63, TP73, TRAF2, TPM3, TPM4, TRA, TRB, TRD, TYK2, U2AF1, UBA2, VAV1, WT1, XPO1, YPEL5, ZCCHC7, ZFP36L2, ZNF362, ZNF384, and ZRSR2.

It is understood and herein contemplated that the genes targeted and/or detected in the disclosed dual DNA-based and RNA-based NGS assays will vary depending on the classification of hematological neoplasms. For example, disclosed herein are dual DNA-based and RNA-based NGS assays, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2. In one aspect, disclosed herein are dual DNA-based and RNA-based NGS assays, wherein the gene expressed in MDS is selected from ASXL1, ATM, BCOR, CALR, CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

Also disclosed herein are dual DNA-based and RNA-based NGS assays, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

In one aspect, disclosed herein are dual DNA-based and RNA-based NGS assays, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

Also disclosed herein are dual DNA-based and RNA-based NGS assays, wherein the gene expressed in CLL is selected from ATM, BCL2, BTK, NOTCH1, PLCG2, RB1, SF3B1, and TP53.

In one aspect, disclosed herein are dual DNA-based and RNA-based NGS assays, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

It is understood and herein contemplated that the disclosed assay can also incorporate the detection of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 375, 400, 425, 450, 475, 500, 525, 550, 575, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000 or more of the single nucleotide variants (i.e., single nucleotide polymorphisms) described in Table 29. Thus, in one aspect, disclosed herein are dual DNA-based and RNA-based NGS assays, wherein the DNA component of the NGS assay detects 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 375, 400, 425, 450, 475, 500, 525, 550, 575, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000 or more single nucleotide variations listed in Table 29.

Also disclosed herein are dual DNA-based and RNA-based NGS assays, wherein the DNA component of the NGS assay simultaneously detects a gene variant and the cytogenetic alternation selected from the group consisting of an EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, an odd number of chromosomes, and trisomy 21.

In one aspect, disclosed herein are dual DNA-based and RNA-based NGS assays, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 1 q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, an odd number of chromosomes, or trisomy 21.

It should be understood that the NGS assay can detect multiple changes in a nucleic acid sequence including, but not limited to deletions, additions, insertions, or a variant or derivative thereof. Further NGS can detect known or unknown deletions, insertions/addition, or a variant or derivative thereof. A deletion of a gene comprises removing a part of a gene or an entire/complete gene. A deletion also comprises removal of large segments of a chromosome. In some embodiments, a deletion comprises removal of at least one or more nucleotides from a gene. In some embodiments, a deletion comprises removal of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62.63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411.412, 413, 414, 415, 416, 417, 418, 419, 420.421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, or more nucleotides.

In some embodiments, the NGS assay can detect deletion of segments of a chromosome. In some embodiments, the NGS assay can detect deletion of an entire chromosome.

An addition/insertion comprises adding or inserting nucleotides, oligonucleotides, or polynucleotides into a gene. An addition/insertion can also be a duplication, wherein a segment of a gene or an entire/complete gene is repeated contiguously. In some embodiments, an addition/insertion comprises adding at least one or more nucleotides to a gene. In some embodiments, an addition/insertion comprises adding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, or more nucleotides.

In some embodiments, a duplication comprises repeating a gene at least one time. In some embodiments, a duplication comprises repeating a gene 1, 2, 3, 4, 5, 6, 7, 8, 9, 10 or more.

A “single nucleotide variant”, “SNV”, or “single nucleotide polymorphism” refers to a nucleic acid sequence variation wherein a single nucleotide (adenine, thymine, cytosine, or guanine) in the genome sequence is altered into another nucleotide. In some embodiments, the nucleic acid sequence comprises a single nucleotide variant.

An “insertion-deletion” or “indel” refers to nucleic acid variant wherein a deletion and an insertion occur at the same time at the same genomic location, possibly resulting in a protein not folding or functioning properly. Therefore, an “insertion-deletion” variant requires at least nucleotide to be removed and at least one nucleotide to be added. In some embodiments, the nucleic acid sequence comprises an insertion-deletion variant.

A “chimeric fusion” variant refers to a nucleic acid formed when two or more genes (usually from two distinct genomic locations) combine into one new gene, and express proteins with functions differing from that of the parent proteins. In some embodiments, the nucleic acid sequence comprises a chimeric fusion variant.

A “structural variant” refers to a nucleic acid, usually 1000 base pairs, or 1 kilobases (kb), in size, wherein the overall chromosomal structure is altered. “Structural variations” can further comprise other variations, including, but not limited to deletions, insertions, indels, copy number variations, duplications, inversion, substitutions, and derivatives thereof. In some embodiments, the nucleic acid comprises a structural variant.

A “copy number variant” refers to nucleic acid sequence wherein segment(s) of the genome are repeated at least one time, and the number of repeats in the genome varies between species and individuals with a species. A “copy number variant” is also a specific type of “structural variant”. In some embodiments, the nucleic acid sequence comprises a copy number variant.

In some embodiments, the nucleic acid sequence comprises a variant selected from a group consisting of a single nucleotide variant, an insertion deletion (indel), a chimeric fusion, a structural variant, and a copy number variant.

In some embodiments, the NGS assay simultaneously detects more than one variant within the hematological neoplasm.

In some embodiments, the NGS assay detects one or more variants in a gene affected by a hematological neoplasm or a blood-related cancer.

Gene libraries are collections of total genomic DNA from an individual subject that can be used to generate a genetic profile for the said subject. Such libraries are a part of personalized medicine strategies to help guide practitioners and medical professionals to make diagnostic and therapeutic decisions. Building such libraries requires reagents, including, but not limited to expression vectors, storage buffers, nuclease enzymes, and ligation enzymes. Accordingly, in one aspect, the sequencing assay further comprises a gene library construction reagent. In some embodiments, the gene library construction reagent(s) include, but are not limited to an expression vector, an endonuclease or nuclease enzyme, a nucleotide solution, a ligase enzyme, and storage and reaction buffers, including, but not limited to Tris, TE, water, or saline.

The term “polymerase chain reaction” (“PCR”) refers to a method for increasing or amplifying the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multiplex-PCR, reverse transcription PCR, or any of the other PCR variants known to those skilled in the art. Reagents used to perform PCRs include, but are not limited to a polymerase enzyme, storage and reaction buffers, and a nucleotide solution.

In some embodiments, the NGS assay further comprises a gene amplification reagent. In some embodiments, the gene amplification reagent is a PCR reagent. In some embodiments, the gene amplification reagent(s) includes, but is not limited to an RNA or DNA polymerase enzyme, a nucleotide solution, and storage and reaction buffers, including, but not limited to Tris, TE, water, or saline.

In some embodiments, the NGS assay further comprises a verification sample comprising a nucleic acid standard composition encoding one or more genes selected from ABL1, ABL2, ACTB, AFF1, AHI1, ALK, ASXL1, ASXL2, ATF7IP, ATM, BCL11B, BCL2, BCL6, BCOR, BCR, BIRC3, BMI1, BTK, CALR, CBFB, CBFA2T3, CBL, CCND1, CCND3, CD28, CDK6, CDKN2C, CEP43, CEBPA, CHD1, CHEK2, CHIC2, CIITA, CREBBP, CRLF2, CSF1R, CUX1, CXCR4, DAZAP1, DDX6, DDX10, DDX3X, DDX41, DEK, DIS3, DNMT3A, DSCAM, DUSP22, EBF1, EGR1, EIF4A1, ELL, EP300, EPOR, ERG, ETS2, ETV6, FGFR1, FGFR3, FLT3, FOXP1, FOXR1, FUS, GATA2, GLIS2, H2AX, HNRNPC, HOXA9, HOXA11, IDH1, IDH2, IGH, IGK, IGL, IKZF1, IKZF2, IKZF3, IRF4, JAK2, KANSL1, KAT6A, KDM5A, KLF2, KMT2A, KMT2C, KRAS, LMO1, LMO2, LYN, MALT1, MAP2K1, MECOM, MEK, MLF1, MLLT4, MLLT10, MKL1, MPL, MN1, MPO, MRTFA, MSI2, MTCP1, MYB, MYC, MYH9, MYH11, NF1, NFIA, NFE2, NFKB2, NIPBL, NOTCH1, NPM1, NRAS, NSD1, NTRK3, NUP214, NUP98, NUTM1, P2RY8, PAG1, PAX5, PBX1, PDCD1LG2, PDGFRA, PDGFRB, PHF6, PICALM, PLCG2, PML, PPM1D, PRDM16, PRRC2B, PTK2B, RARA, RARG, RB1, RBM15, RET, ROS1, RNF213, RUNX1, RUNX1T1, SEC31A, SETBP1, SEMA6A, SEPTIN9, SETD2, SF3B1, SH2B3, SPI1, SRSF2, SSBP2, STAG2, STAT3, STAT6, STIL, SYK, TAL1, TBL1XR1, TCF3, TCL1A, TFG, TP63, TP73, TRAF2, TPM3, TPM4, TRA, TRB, TRD, TYK2, U2AF1, UBA2, VAV1, WT1, XPO1, YPEL5, ZCCHC7, ZFP36L2, ZNF362, ZNF384, and ZRSR2.

In addition to the DNA-based sequencing, the disclosed dual NGS assays utilize an RNA-based component. The RNA component of the assay was designed and developed using specific databases to identify key fusion genes important to hematological malignancies. This custom RNA hybridization capture assay allows for the fusion analysis of 125 genes associated with hematological malignancies without the need for prior knowledge of the fusion partners or the breakpoints of the translocations when a chimeric transcript is formed.

In one aspect, disclosed herein are dual DNA-based and RNA-based NGS assays, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CIITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCH1, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TAL1, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUX1, SEPTIN9, TP63, SPI1, RNF213, TCL1A, ELL, DDX3X, or HNRNPC.

Also disclosed herein are dual DNA-based and RNA-based NGS assays, wherein the chimeric fusion comprises CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are dual DNA-based and RNA-based NGS assays, wherein the chimeric fusion in mantel cell lymphoma (MCL) comprises CCND1-IGH.

Also disclosed herein are dual DNA-based and RNA-based NGS assays, wherein the chimeric fusion in MM comprises CCND1-IGH or FGFR3-IGH.

In one aspect, disclosed herein are dual DNA-based and RNA-based NGS assays, wherein the chimeric fusion in diffuse large B-cell lymphoma (DLBCL) comprises BCL6-IGH or BCL2-IGH.

Also disclosed herein are dual DNA-based and RNA-based NGS assay of any preceding aspect, wherein the chimeric fusion in follicular lymphoma (FL) comprises BCL6-IGH, BCL2-IGH, or MYC-IGH.

In one aspect, disclosed herein are dual DNA-based and RNA-based NGS assays, wherein the chimeric fusion in Burkitt lymphoma comprises MYC-IGH.

Also disclosed herein are dual DNA-based and RNA-based NGS assays, wherein the chimeric fusion in mucosal-associated lymphoma (MALT) comprises MALT1-IGH or AP12-MALT1.

In one aspect, disclosed herein are dual DNA-based and RNA-based NGS assays, wherein the chimeric fusion in B-acute lymphocytic leukemia (B-ALL) comprises ETV6-RUNX1(TEL-AML1), TCF3-PBX1 (E2A-PBX), ABL1-BCR, or AF1-MLL.

Also disclosed herein are dual DNA-based and RNA-based NGS assays, wherein the chimeric fusion in chronic myelogenous leukemia (CML) comprises ABL1-BCR.

In one aspect, disclosed herein are dual DNA-based and RNA-based NGS assays, wherein the chimeric fusions detected in AML or an AML related therapy comprises ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, or RUNX1-ZFPM2.

Also disclosed herein are dual DNA-based and RNA-based NGS assays, wherein the chimeric fusions detected in MDS comprise CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are dual DNA-based and RNA-based NGS assays, wherein the chimeric fusions detected in MPN comprise KANK1-PDGFRB, ETV6-LYN, PCM1-JAK2, PDE4DIP-PDGFRB, or GOLGIB1-PDGFRB.

Also disclosed herein are dual DNA-based and RNA-based NGS assays, wherein the chimeric fusion detected in NHL comprises CCND1-IGH, FGFR3-IGH, BCL6-IGH, BCL2-IGH, MYC-IGH, MALT1-IGH, or API2-MALT1.

As used herein, a “verification sample”, also known as a “positive control” sample refers to a nucleic acid sample of known content, concentration, and volume, often referred to as “normal”, used as a standard comparison to a test sample, or in this case the nucleic acid sequence comprising the variant.

In some embodiments, the detection probe emits a light or color signal when the oligonucleotide interacts the nucleic acid sequence. In some embodiments, the detection probe is a fluorescent probe, a chemiluminescent probe, a luminescent probe, a fluorescent protein, or a light/color emitting compound.

In some embodiments, the detection probe is a fluorophore including, but not limited to Alexa Fluor 350, Alexa Fluor 405, Alexa Fluor 488, Alexa Fluor 532, Alexa Fluor 546, Alexa Fluor 555, Alexa Fluor 561, Alexa Fluor 568, Alexa Fluor 594, Alexa Fluor 647, Alexa Fluor 660, Alexa Fluor 680, Alexa Fluor 700, Alexa Fluor 750, BODIPY FL, Coumarin, Cy3, Cy5, Fluorescein (FITC), Oregon Green, Pacific Blue, Pacific Green, Pacific Orange, PE-Cvanine7, PerCP-Cyanine5.5, Tetramethylrhodamine (TRITC), Texas Red, DAPI, Propidium Iodide, SYTO 9, SYTOX Green, TO-PRO-3, Allophycocyanin (APC), R-Phycoerythrin (R-PE), Cyan Fluorescent Protein (CFP), Green Fluorescent Protein (GFP), Red Fluorescent Protein (RFP), and/or Blue Fluorescent Protein (BFP).

In some embodiments, the detection probe is a fluorescent protein including, but not limited to luciferase, green fluorescent protein (GFP), yellow fluorescent protein (YFP), blue fluorescent protein (BFP), cyane fluorescent protein (CFP), monomeric red fluorescent protein (mRFP). Discosoma striata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

### Methods of Identifying or Profiling Hematological Neoplasms

The present disclosure provides a method of identifying or profiling hematological neoplasms in a subject.

In one aspect, disclosed herein are methods of identifying or profiling a hematological neoplasm in a subject in need thereof, the method comprising a) obtaining a tissue sample from the subject, b) performing the dual DNA-based and RNA-based NGS assay, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). For example, disclosed herein are methods of identifying or profiling a hematological neoplasm in a subject in need thereof, the method comprising a) obtaining a tissue sample from the subject; b) performing dual DNA-based and RNA-based next generation sequencing (NGS) assay on the tissue sample using an oligonucleotide and a detection probe targeting DNA isolated from the tissue sample and an oligonucleotide and a detection probe targeting RNA isolated from the tissue sample, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). In some aspects, the nucleic acid sequence detected by the assay encodes BRAF, EZH2, KIT, MYD88, TP53, TET2, or a gene expressed in AML, MDS, MPN, NHL, CLL, or MM.

In one aspect, disclosed herein are methods of identifying or profiling a hematological neoplasm of any preceding aspect, wherein the nucleic acid sequence targeted by the DNA component encodes a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUXL, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO1, FBXW7, FGFR1, FGFR2, FGFR3, FLT3, FOXO1, FYN, G6PC3, GATA1, GATA2, GATA3, GFI1, GNA13, GNAS, GPR34, GRB2, GTF2I, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NF1, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2. It is understood and herein contemplated that the DNA component is not limited to the detection of a single nucleic acid, but can detect multiple nucleic acids simultaneously with the use of multiple oligonucleotides and probes. Accordingly, in one aspect, the DNA component of the dual DNA-based and RNA-based NGS assay detects at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, or all 345 of said heme-related genes.

In some embodiments, the AML includes, but is not limited to acute lymphoblastic leukemia (ALL), Lymphoblastic (diffuse) lymphoma, acute myeloblastic leukemia, chronic lymphoblastic leukemia of B cell type, myeloid leukemia, atypical chronic myeloid leukemia BCR/ABL—negative, myeloid sarcoma, acute promyelocytic leukemia, acute myelomonocytic leukemia, acute myeloid leukemia with 11q23-abnormality, chronic myeloid leukemia (CML), B-acute lymphocytic leukemia (B-ALL), and acute myeloid leukemia with multidimensional dysplasia.

In some embodiments, the MDS includes, but is not limited to refractory anemia, refractory cytopenia with multilineage dysplasia, refractory anemia with ringed sideroblasts, refractory cytopenia with multilineage dysplasia and ringed sideroblasts, and refractory anemia with excess blasts.

In some embodiments, the MPN includes, but is not limited to polycythemia vera, chronic myeloproliferative disease, and essential (hemorrhagic) thrombocythemia.

In some embodiments, the NHL includes, but is not limited to small cell B cell lymphoma (splenic marginal zone lymphoma), mantle cell lymphoma, multiple myeloma, diffuse large B cell lymphoma, peripheral T cell lymphoma (not classified), Waldenstrom macroglobulinemia (WM), follicular lymphoma, Burkitt lymphoma, mucosal-associated lymphoma, and post-transplant lymphoproliferative disorder (PTLD).

In some embodiments, the CLL includes, but is not limited to chronic lymphocytic leukemia of B cell type, small lymphocytic lymphoma (SLL), prolymphocytic leukemia (PLL), large granular lymphocyte (LGL) leukemia, and hairy cell leukemia (HCL).

In some embodiments, the MM includes, but is not limited to smoldering multiple myeloma (SMM), light chain amyloidosis, and Waldenstrom macroglobulinemia (WM).

In some embodiments, the method comprises a DNA-based NGS assay or an RNA-based NGS assay. In some embodiments, the method comprises hybridizing a complementary oligonucleotide to a DNA sequence isolated from the tissue sample. In some embodiments, the method comprises fusing a complementary or non-complementary oligonucleotide to an RNA sequence isolated from the tissue sample. In some embodiments, the oligonucleotides comprises 15, 16, 17, 18, 19, 20, 21, 22, 23, 24, 25, or more nucleotides.

Also disclosed herein are methods of identifying or profiling a hematological neoplasm, wherein the NGS assay detects a variant selected from a group consisting of a single nucleotide variant, an insertion deletion (indel), a chimeric fusion, a structural variant, a copy number variant, and a chromosomal abnormality.

In one aspect, disclosed herein are methods of identifying or profiling a hematological neoplasm of any preceding aspect, wherein the nucleic acid sequence encodes BRAF, EZH2, KIT, MYD88, TP53, TET2, or a gene expressed in AML, MDS, MPN, NHL, CLL, or MM.

In one aspect, disclosed herein are methods of identifying or profiling a hematological neoplasm, wherein the NGS assay simultaneously detects more than one variants within the hematological neoplasm.

Also disclosed herein are methods of identifying or profiling a hematological neoplasm, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2.

In one aspect, disclosed herein are methods of identifying or profiling a hematological neoplasm, wherein the gene expressed in MDS is selected from ASXL1, ATM, BCOR, CALR. CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

Also disclosed herein are methods of identifying or profiling a hematological neoplasm, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

In one aspect, disclosed herein are methods of identifying or profiling a hematological neoplasm, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

Also disclosed herein are methods of identifying or profiling a hematological neoplasm, wherein the gene expressed in CLL is selected from ATM, BCL2, BTK, NOTCH1, PLCG2, RB1, SF3B1, and TP53.

In one aspect, disclosed herein are methods of identifying or profiling a hematological neoplasm, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

Also disclosed herein are methods of identifying or profiling a hematological neoplasm, wherein the DNA component of the NGS assay detects a gene variant and the chromosomal abnormality selected from EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, and an odd number of chromosomes.

In one aspect, disclosed herein are methods of identifying or profiling a hematological neoplasm, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9. PTEN variant and deletion of chromosome segment 10q23, RB1 variant and deletion of chromosome segment 13q14, POT1 variant and deletion of chromosome segment 7q31, duplication of chromosome 12, or an odd number of chromosomes.

It is understood and herein contemplated that the dual DNA-based and RNA-based NGS assays used in the disclosed methods of identifying or profiling a hematological neoplasm can also incorporate the detection of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 375, 400, 425, 450, 475, 500, 525, 550, 575, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000 or more of the single nucleotide variants (i.e., single nucleotide polymorphisms) described in Table 29. Thus, in one aspect, disclosed herein are methods of identifying or profiling a hematological neoplasm, wherein the DNA component of the NGS assay detects 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 375, 400, 425, 450, 475, 500, 525, 550, 575, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000 or more single nucleotide variations listed in Table 29.

Also disclosed herein are methods of identifying or profiling a hematological neoplasm, wherein the RNA component of the NGS assay detects a chimeric gene fusion without prior knowledge of the fusion partners or the breakpoint translocation.

In one aspect, disclosed herein are methods of identifying or profiling a hematological neoplasm, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CIITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCHL, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TAL1, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUX1, SEPTIN9, TP63, SPI1, RNF213, TCL1A, ELL, DDX3X, or HNRNPC Also disclosed herein are methods of identifying or profiling a hematological neoplasm, wherein the chimeric fusion comprises CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of identifying or profiling a hematological neoplasm, wherein the chimeric fusion in mantel cell lymphoma (MCL) comprises CCND1-IGH.

Also disclosed herein are methods of identifying or profiling a hematological neoplasm, wherein the chimeric fusion in MM comprises CCND1-IGH or FGFR3-IGH.

In one aspect, disclosed herein are methods of identifying or profiling a hematological neoplasm, wherein the chimeric fusion in diffuse large B-cell lymphoma (DLBCL) comprises BCL6-IGH or BCL2-IGH.

Also disclosed herein are methods of identifying or profiling a hematological neoplasm, wherein the chimeric fusion in follicular lymphoma (FL) comprises BCL6-IGH, BCL2-IGH, or MYC-IGH.

In one aspect, disclosed herein are methods of identifying or profiling a hematological neoplasm, wherein the chimeric fusion in Burkitt lymphoma comprises MYC-IGH.

Also disclosed herein are methods of identifying or profiling a hematological neoplasm, wherein the chimeric fusion in mucosal-associated lymphoma (MALT) comprises MALT1-IGH or API2-MALT1.

In one aspect, disclosed herein are methods of identifying or profiling a hematological neoplasm, wherein the chimeric fusion in B-acute lymphocytic leukemia (B-ALL) comprises ETV6-RUNX1(TEL-AML1), TCF3-PBX1 (E2A-PBX), ABL1-BCR, or AF1-MLL.

Also disclosed herein are methods of identifying or profiling a hematological neoplasm, wherein the chimeric fusion in chronic myelogenous leukemia (CML) comprises ABL1-BCR.

In one aspect, disclosed herein are methods of identifying or profiling a hematological neoplasm, wherein the chimeric fusions detected in AML or an AML related therapy comprises ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, or RUNX1-ZFPM2.

Also disclosed herein are methods of identifying or profiling a hematological neoplasm, wherein the chimeric fusions detected in MDS comprise CCND1-IGH, FGFR3-IGH, BCL6-IGH, BCL2-IGH, MYC-IGH, MALT1-IGH, API2-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of identifying or profiling a hematological neoplasm, wherein the chimeric fusions detected in MPN comprise KANK1-PDGFRB, ETV6-LYN, PCM1-JAK2, PDE4DIP-PDGFRB, or GOLGIB1-PDGFRB.

Also disclosed herein are methods of identifying or profiling a hematological neoplasm, wherein the chimeric fusion detected in NHL comprises CCND1-IGH, FGFR3-IGH, BCL6-IGH, BCL2-IGH, MYC-IGH, MALT1-IGH, or API2-MALT1.

In some embodiments, the variant is detected by a light or color emitting probe when the oligonucleotide interacts with the nucleic acid sequence. In some embodiments, the detection probe is a fluorescent probe, a chemiluminescent probe, a luminescent probe, a fluorescent protein, or a light/color emitting compound.

In some embodiments, the detection probe is a fluorophore including, but not limited to Alexa Fluor 350, Alexa Fluor 405, Alexa Fluor 488, Alexa Fluor 532, Alexa Fluor 546, Alexa Fluor 555, Alexa Fluor 561, Alexa Fluor 568, Alexa Fluor 594, Alexa Fluor 647, Alexa Fluor 660, Alexa Fluor 680, Alexa Fluor 700, Alexa Fluor 750, BODIPY FL, Coumarin, Cy3, Cy5, Fluorescein (FITC), Oregon Green, Pacific Blue, Pacific Green, Pacific Orange, PE-Cyanine7, PerCP-Cyanine5.5, Tetramethylrhodamine (TRITC), Texas Red, DAPI, Propidium Iodide, SYTO 9, SYTOX Green, TO-PRO-3, Allophycocyanin (APC), R-Phycoerythrin (R-PE), Cyan Fluorescent Protein (CFP), Green Fluorescent Protein (GFP), Red Fluorescent Protein (RFP), and/or Blue Fluorescent Protein (BFP).

In some embodiments, the detection probe is a fluorescent protein including, but not limited to luciferase, green fluorescent protein (GFP), yellow fluorescent protein (YFP), blue fluorescent protein (BFP), cyane fluorescent protein (CFP), monomeric red fluorescent protein (mRFP), Discosoma striata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

In some embodiments, the tissue sample is a blood, bone marrow, serum, or fluid sample.

In some embodiments, the subject is a mammal. In some embodiments, the subject is a human.

### Methods of Treating a Hematological Neoplasm

In one aspect, disclosed herein are methods of treating, inhibiting, reducing, decreasing, ameliorating, and/or preventing a hematological neoplasm in a subject in need thereof, the method comprising a) obtaining a tissue sample from the subject, b) performing the dual DNA-based and RNA-based NGS assay of any preceding aspect, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). For example, disclosed herein are methods of treating, inhibiting, reducing, decreasing, ameliorating, and/or preventing a hematological neoplasm in a subject in need thereof, the method comprising a) obtaining a tissue sample from the subject; b) performing dual DNA-based and RNA-based next generation sequencing (NGS) assay on the tissue sample using an oligonucleotide and a detection probe targeting DNA isolated from the tissue sample and an oligonucleotide and a detection probe targeting RNA isolated from the tissue sample, and c) detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). In some aspects, the nucleic acid sequence detected by the assay encodes BRAF, EZH2, KIT, MYD88, TP53, TET2, or a gene expressed in AML, MDS, MPN, NHL, CLL, or MM.

Also disclosed herein are methods of treating, inhibiting, reducing, decreasing, ameliorating, and/or preventing a hematological neoplasm wherein the nucleic acid sequence targeted by the DNA component encodes a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF2I, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NFL, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2. It is understood and herein contemplated that the DNA component is not limited to the detection of a single nucleic acid, but can detect multiple nucleic acids simultaneously with the use of multiple oligonucleotides and probes. Accordingly, in one aspect, the DNA component of the dual DNA-based and RNA-based NGS assay detects at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, or all 345 of said heme-related genes.

In some embodiments, the AML includes, but is not limited to acute lymphoblastic leukemia (ALL), Lymphoblastic (diffuse) lymphoma, acute myeloblastic leukemia, chronic lymphoblastic leukemia of B cell type, myeloid leukemia, atypical chronic myeloid leukemia BCR/ABL—negative, myeloid sarcoma, acute promyelocytic leukemia, acute myelomonocytic leukemia, acute myeloid leukemia with 11q23-abnormality, chronic myeloid leukemia (CML), B-acute lymphocytic leukemia (B-ALL), and acute myeloid leukemia with multidimensional dysplasia.

In some embodiments, the MDS includes, but is not limited to refractory anemia, refractory cytopenia with multilineage dysplasia, refractory anemia with ringed sideroblasts, refractory cytopenia with multilineage dysplasia and ringed sideroblasts, and refractory anemia with excess blasts.

In some embodiments, the MPN includes, but is not limited to polycythemia vera, chronic myeloproliferative disease, and essential (hemorrhagic) thrombocythemia.

In some embodiments, the NHL includes, but is not limited to small cell B cell lymphoma (splenic marginal zone lymphoma), mantle cell lymphoma, multiple myeloma, diffuse large B cell lymphoma, peripheral T cell lymphoma (not classified), Waldenstrom macroglobulinemia (WM), follicular lymphoma, Burkitt lymphoma, mucosal-associated lymphoma, and post-transplant lymphoproliferative disorder (PTLD).

In some embodiments, the CLL includes, but is not limited to chronic lymphocytic leukemia of B cell type, small lymphocytic lymphoma (SLL), prolymphocytic leukemia (PLL), large granular lymphocyte (LGL) leukemia, and hairy cell leukemia (HCL).

In some embodiments, the MM includes, but is not limited to smoldering multiple myeloma (SMM), light chain amyloidosis, and Waldenstrom macroglobulinemia (WM).

Also disclosed herein are methods of treating, inhibiting, reducing, decreasing, ameliorating, and/or preventing a hematological neoplasm, wherein the NGS assay detects a variant selected from a group consisting of a single nucleotide variant, an insertion deletion (indel), a chimeric fusion, a structural variant, a copy number variant, and a chromosomal abnormality.

In one aspect, disclosed herein are methods of treating, inhibiting, reducing, decreasing, ameliorating, and/or preventing a hematological neoplasm of any preceding aspect, wherein the nucleic acid sequence encodes BRAF, EZH2, KIT, MYD88, TP53, TET2, or a gene expressed in AML, MDS, MPN, NHL, CLL, or MM.

In one aspect, disclosed herein are methods of treating, inhibiting, reducing, decreasing, ameliorating, and/or preventing a hematological neoplasm, wherein the NGS assay simultaneously detects more than one variants within the hematological neoplasm.

Also disclosed herein are methods of treating, inhibiting, reducing, decreasing, ameliorating, and/or preventing a hematological neoplasm, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2. In one aspect, disclosed herein are methods of treating, inhibiting, reducing, decreasing, ameliorating, and/or preventing a hematological neoplasm, wherein the gene expressed in MDS is selected from ASXL1, ATM, BCOR, CALR, CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

Also disclosed herein are methods of treating, inhibiting, reducing, decreasing, ameliorating, and/or preventing a hematological neoplasm, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

In one aspect, disclosed herein are methods of treating, inhibiting, reducing, decreasing, ameliorating, and/or preventing a hematological neoplasm, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

Also disclosed herein are methods of treating, inhibiting, reducing, decreasing, ameliorating, and/or preventing a hematological neoplasm, wherein the gene expressed in CLL is selected from ATM, BCL2, BTK, NOTCH1, PLCG2, RB1, SF3B1, and TP53.

In one aspect, disclosed herein are methods of treating, inhibiting, reducing, decreasing, ameliorating, and/or preventing a hematological neoplasm, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

Also disclosed herein are methods of treating, inhibiting, reducing, decreasing, ameliorating, and/or preventing a hematological neoplasm, wherein the DNA component of the NGS assay detects a gene variant and the chromosomal abnormality selected from EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, and an odd number of chromosomes.

In one aspect, disclosed herein are methods of treating, inhibiting, reducing, decreasing, ameliorating, and/or preventing a hematological neoplasm, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, or an odd number of chromosomes.

It is understood and herein contemplated that the dual DNA-based and RNA-based NGS assays used in the disclosed methods of treating, inhibiting, reducing, decreasing, ameliorating, and/or preventing a hematological neoplasm can also incorporate the detection of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 375, 400, 425, 450, 475, 500, 525, 550, 575, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000 or more of the single nucleotide variants (i.e., single nucleotide polymorphisms) described in Table 29. Thus, in one aspect, disclosed herein are methods of treating, inhibiting, reducing, decreasing, ameliorating, and/or preventing a hematological neoplasm, wherein the DNA component of the NGS assay detects 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 375, 400, 425, 450, 475, 500, 525, 550, 575, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000 or more single nucleotide variations listed in Table 29.

Also disclosed herein are methods of treating, inhibiting, reducing, decreasing, ameliorating, and/or preventing a hematological neoplasm, wherein the RNA component of the NGS assay detects a chimeric gene fusion without prior knowledge of the fusion partners or the breakpoint translocation.

In one aspect, disclosed herein are methods of treating, inhibiting, reducing, decreasing, ameliorating, and/or preventing a hematological neoplasm, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CIITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCHL, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TALL, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUX1, SEPTIN9, TP63, SPI1, RNF213, TCL1A, ELL, DDX3X, or HNRNPC

Also disclosed herein are methods of treating, inhibiting, reducing, decreasing, ameliorating, and/or preventing a hematological neoplasm, wherein the chimeric fusion comprises CCND1-IGH, FGFR3-IGH, BCL6-IGH, BCL2-IGH, MYC-IGH, MALT1-IGH, AP12-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of treating, inhibiting, reducing, decreasing, ameliorating, and/or preventing a hematological neoplasm, wherein the chimeric fusion in mantel cell lymphoma (MCL) comprises CCND1-IGH.

Also disclosed herein are methods of treating, inhibiting, reducing, decreasing, ameliorating, and/or preventing a hematological neoplasm, wherein the chimeric fusion in MM comprises CCND1-IGH or FGFR3-IGH.

In one aspect, disclosed herein are methods of treating, inhibiting, reducing, decreasing, ameliorating, and/or preventing a hematological neoplasm, wherein the chimeric fusion in diffuse large B-cell lymphoma (DLBCL) comprises BCL6-IGH or BCL2-IGH.

Also disclosed herein are methods of treating, inhibiting, reducing, decreasing, ameliorating, and/or preventing a hematological neoplasm, wherein the chimeric fusion in follicular lymphoma (FL) comprises BCL6-IGH, BCL2-IGH, or MYC-IGH.

In one aspect, disclosed herein are methods of treating, inhibiting, reducing, decreasing, ameliorating, and/or preventing a hematological neoplasm, wherein the chimeric fusion in Burkitt lymphoma comprises MYC-IGH.

Also disclosed herein are methods of treating, inhibiting, reducing, decreasing, ameliorating, and/or preventing a hematological neoplasm, wherein the chimeric fusion in mucosal-associated lymphoma (MALT) comprises MALT1-IGH or API2-MALT1.

In one aspect, disclosed herein are methods of treating, inhibiting, reducing, decreasing, ameliorating, and/or preventing a hematological neoplasm, wherein the chimeric fusion in B-acute lymphocytic leukemia (B-ALL) comprises ETV6-RUNX1(TEL-AML1), TCF3-PBX1 (E2A-PBX), ABL1-BCR, or AF1-MLL.

Also disclosed herein are methods of treating, inhibiting, reducing, decreasing, ameliorating, and/or preventing a hematological neoplasm, wherein the chimeric fusion in chronic myelogenous leukemia (CML) comprises ABL1-BCR.

In one aspect, disclosed herein are methods of treating, inhibiting, reducing, decreasing, ameliorating, and/or preventing a hematological neoplasm, wherein the chimeric fusions detected in AML or an AML related therapy comprises ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, or RUNX1-ZFPM2.

Also disclosed herein are methods of treating, inhibiting, reducing, decreasing, ameliorating, and/or preventing a hematological neoplasm, wherein the chimeric fusions detected in MDS comprise CCND1-IGH, FGFR3-IGH, BCL6-IGH, BCL2-IGH, MYC-IGH, MALT1-IGH, AP12-MALT1, ETV6-RUNX1(TEL-AML1), TCF3-PBX1(E2A-PBX), AF1-MLL, ABL1-BCR, RUNX1-RUNX1T1(AML1-ETO), PML-RARA, MYH11-CBFB, ETV6-ACSL6, ETV6-SYK, KAT6B-CREBBP, KMT2A-ACTN4, KMT2A-CREBBP, KMT2A-ELL, KMT2A-MLLT3, KMT2A-MYH11, RUNX1-CBFA2T3, RUNX1-MECOM, RUNX1-USP42, RUNX1-ZFPM2, KANK1-PDGFRB, ETV6-LYN, PCM1-JAK2, PDE4DIP-PDGFRB, GOLGIB1-PDGFRB, or DEK-NUP214.

In one aspect, disclosed herein are methods of treating, inhibiting, reducing, decreasing, ameliorating, and/or preventing a hematological neoplasm, wherein the chimeric fusions detected in MPN comprise KANK1-PDGFRB, ETV6-LYN, PCM1-JAK2, PDE4DIP-PDGFRB, or GOLGIB1-PDGFRB.

Also disclosed herein are methods of treating, inhibiting, reducing, decreasing, ameliorating, and/or preventing a hematological neoplasm, wherein the chimeric fusion detected in NHL comprises CCND1-IGH, FGFR3-IGH, BCL6-IGH, BCL2-IGH, MYC-IGH, MALT1-IGH, or API2-MALT1.

In one aspect, disclosed herein are methods of treating, inhibiting, reducing, decreasing, ameliorating, and/or preventing a hematological neoplasm, wherein the detection probe emits a light or a color signal when the oligonucleotide interacts with the nucleic acid sequence.

Also disclosed herein are methods of treating, inhibiting, reducing, decreasing, ameliorating, and/or preventing a hematological neoplasm, wherein the tissue sample is a blood, bone marrow, serum, or fluid sample.

The disclosed methods of treatment can utilize any anti-cancer therapy known in the art appropriate for the treatment, inhibition, reduction, decrease, amelioration, and/or prevention of a particular hematological neoplasm including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine). Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuwumab), Camptosar, (Irinotecan Hydrochloride). Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine). CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab. DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochlonde Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI. FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab). Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diflitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin). Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine. Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: A Custom Next Generation Sequencing Assay for Genomic Profiling for Hematological Neoplasms

For purposes of validation this test has been designated the RP-PanHeme (RPPH) assay. The RP-PanHeme assay is designed to best serve both the clinical and research mission of RPCCC. The panel has been characterized into six generic classifications of hematopoietic neoplasms, including acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM). These are the most useful indications for molecular testing as each of these six groups has associations with specific genomic variants that have primarily prognostic, diagnostic, or theranostic applications. In addition to these 6 general classifications there are specific instances where variants in the gene TP53, MYD88, BRAF, EZH2, TET2, and KIT are specifically requested for specific purposes. The following examples provide a detailed test description of each of these assays as would be provided to providers or payers in a clinical context. All 12 assays have the same report format that is limited to the reported variant, level of evidence for that variant per ASCO/CAP guidelines, and if applicable associated clinical trials open at RPCCC. Therapeutic, diagnostic, or prognostic associations are not part of the report, but can be searched for in a Roswell Park campus-wide genomics knowledge database licensed from Jackson Laboratory. Results for all variants not part of a specific assay are placed into a research database that is available to investigators with appropriate IRB approval that is managed outside of the clinical laboratory by RPCCC designated National Cancer Institute Cancer Center Support Center core facilities. The stratification of specific reporting based on sub-class of disease may not be part of the model when distributed outside of RPCCC.

RPPH is an RNA- and DNA-based targeted next generation sequencing (NGS) assay for which the results can be divided into 5 basic categories that include single nucleotide variants, insertion deletions (indels), copy number variants at gene level, chimeric fusions, and structural variants at a cytogenetic level. The latter of these 5 basic categories of genomic characteristics is possible as the assay includes over 20,000 highly informative polymorphic single nucleotide polymorphisms (SNPs).

The DNA component of RPPH is a custom hybridization capture panel of 345 heme-related genes ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF21, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NF1, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AFL, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2 combined with a genome wide SNP backbone (Table 29) that evaluates single nucleotide variants (SNVs), insertion deletions (indels), and copy number changes at both the gene and cytogenetic level. The design for the 345 heme-related genes is complete exon coverage and for the genome wide SNP backbone there is an average of one amplicon every million base pairs. RPPH was designed to manage the more difficult technological problems such as large internal tandem duplications of 125 to 200 base in FLT3, SNVs in high GC content such as CEBPA, and identification of insertions in homopolymers in genes such as for ASXL1. Additionally, the complete exon coverage allows for the identification of less common mutations in frequently mutated genes such as TP53.

TABLE 29
Agilent SureSelect DNA Genomic
CNV Regions Covered by Probes

| | | |
|---|---|---|
| chr1 | 11601 | 11725 |
| chr1 | 163679 | 163808 |
| chr1 | 174354 | 174476 |
| chr1 | 569440 | 569564 |
| chr1 | 699519 | 699649 |
| chr1 | 744936 | 745068 |
| chr1 | 817800 | 817924 |
| chr1 | 948435 | 948563 |
| chr1 | 1124573 | 1124711 |
| chr1 | 1327713 | 1327847 |
| chr1 | 1478120 | 1478268 |
| chr1 | 1594050 | 1594178 |
| chr1 | 1659935 | 1660063 |
| chr1 | 1718080 | 1718206 |
| chr1 | 1848991 | 1849149 |
| chr1 | 2010651 | 2010773 |
| chr1 | 2192569 | 2192696 |
| chr1 | 2325921 | 2326059 |
| chr1 | 2546187 | 2546313 |
| chr1 | 2577648 | 2577769 |
| chr1 | 2753197 | 2753325 |
| chr1 | 2957548 | 2957670 |
| chr1 | 3072654 | 3072778 |
| chr1 | 3292822 | 3292960 |
| chr1 | 3357964 | 3358088 |
| chr1 | 3594775 | 3594897 |
| chr1 | 3628756 | 3628877 |
| chr1 | 3840788 | 3840912 |
| chr1 | 3996092 | 3996213 |
| chr1 | 4082313 | 4082455 |
| chr1 | 4343840 | 4344010 |
| chr1 | 4462406 | 4462536 |
| chr1 | 4582015 | 4582137 |
| chr1 | 4796185 | 4796309 |
| chr1 | 4965557 | 4965683 |
| chr1 | 5103978 | 5104136 |
| chr1 | 5193585 | 5193709 |
| chr1 | 5332587 | 5332731 |
| chr1 | 5471429 | 5471551 |
| chr1 | 5697051 | 5697173 |
| chr1 | 5744831 | 5745009 |
| chr1 | 5908687 | 5908821 |
| chr1 | 6063603 | 6063733 |
| chr1 | 6243675 | 6243803 |
| chr1 | 6361947 | 6362069 |
| chr1 | 6623409 | 6623533 |
| chr1 | 6715366 | 6715492 |
| chr1 | 6877482 | 6877616 |
| chr1 | 6953635 | 6953763 |
| chr1 | 7123755 | 7123877 |
| chr1 | 7299542 | 7299676 |
| chr1 | 7421804 | 7421942 |
| chr1 | 7637917 | 7638067 |
| chr1 | 7780056 | 7780200 |
| chr1 | 7969989 | 7970123 |
| chr1 | 7996854 | 7996978 |
| chr1 | 8168756 | 8168878 |
| chr1 | 8395040 | 8395174 |
| chr1 | 8497227 | 8497351 |
| chr1 | 8642847 | 8642969 |
| chr1 | 8804943 | 8805081 |
| chr1 | 8999791 | 8999929 |
| chr1 | 9138672 | 9138816 |
| chr1 | 9267839 | 9267979 |
| chr1 | 9380683 | 9380805 |
| chr1 | 9497566 | 9497688 |
| chr1 | 9738696 | 9738822 |
| chr1 | 9885200 | 9885354 |
| chr1 | 10029873 | 10029997 |
| chr1 | 10286286 | 10286410 |
| chr1 | 10463808 | 10463952 |
| chr1 | 10644285 | 10644427 |
| chr1 | 10705828 | 10705954 |
| chr1 | 10911411 | 10911535 |
| chr1 | 10988413 | 10988537 |
| chr1 | 11166639 | 11166793 |
| chr1 | 11374861 | 11375137 |
| chr1 | 11558161 | 11558287 |
| chr1 | 11641247 | 11641371 |
| chr1 | 11750476 | 11750598 |
| chr1 | 12019317 | 12019443 |
| chr1 | 12117843 | 12117989 |
| chr1 | 12277582 | 12277716 |
| chr1 | 12342333 | 12342457 |
| chr1 | 12592196 | 12592330 |
| chr1 | 12714201 | 12714385 |
| chr1 | 12803885 | 12804031 |
| chr1 | 12866305 | 12866449 |
| chr1 | 12908801 | 12908925 |
| chr1 | 12942248 | 12942367 |
| chr1 | 13001387 | 13001506 |
| chr1 | 13219573 | 13219727 |
| chr1 | 13365697 | 13365851 |
| chr1 | 13781549 | 13781670 |
| chr1 | 13931921 | 13932071 |
| chr1 | 14087030 | 14087154 |
| chr1 | 14173226 | 14173366 |
| chr1 | 14297667 | 14297791 |
| chr1 | 14558036 | 14558158 |
| chr1 | 14629562 | 14629684 |
| chr1 | 14852328 | 14852452 |
| chr1 | 14981768 | 14981892 |
| chr1 | 15026178 | 15026318 |
| chr1 | 15208830 | 15208958 |
| chr1 | 15410656 | 15410780 |
| chr1 | 15496433 | 15496563 |
| chr1 | 15733338 | 15733466 |
| chr1 | 15900479 | 15900607 |
| chr1 | 15944530 | 15944668 |
| chr1 | 16196685 | 16196809 |
| chr1 | 16331895 | 16332017 |
| chr1 | 16393146 | 16393370 |
| chr1 | 16593977 | 16594105 |
| chr1 | 16686704 | 16686828 |
| chr1 | 16878277 | 16878397 |
| chr1 | 16890104 | 16890248 |
| chr1 | 16968844 | 16968968 |
| chr1 | 17009292 | 17009446 |
| chr1 | 17051005 | 17051275 |
| chr1 | 17182530 | 17182650 |
| chr1 | 17206377 | 17206531 |
| chr1 | 17249285 | 17249413 |
| chr1 | 17289823 | 17289953 |
| chr1 | 17303537 | 17303659 |
| chr1 | 17386779 | 17386901 |
| chr1 | 17437278 | 17437400 |
| chr1 | 17600241 | 17600373 |
| chr1 | 17794115 | 17794255 |
| chr1 | 17876351 | 17876477 |
| chr1 | 18084250 | 18084374 |
| chr1 | 18263696 | 18263826 |
| chr1 | 18408705 | 18408855 |
| chr1 | 18577785 | 18577921 |
| chr1 | 18632314 | 18632438 |
| chr1 | 18895761 | 18895907 |
| chr1 | 18965930 | 18966070 |
| chr1 | 19169916 | 19170038 |
| chr1 | 19304549 | 19304693 |
| chr1 | 19480755 | 19480877 |
| chr1 | 19535092 | 19535220 |
| chr1 | 19809706 | 19809832 |
| chr1 | 19849594 | 19849742 |
| chr1 | 19978859 | 19978983 |
| chr1 | 20188414 | 20188542 |
| chr1 | 20359126 | 20359274 |
| chr1 | 20451752 | 20451882 |
| chr1 | 20582599 | 20582735 |
| chr1 | 20872614 | 20872744 |
| chr1 | 20977968 | 20978116 |
| chr1 | 21069055 | 21069177 |
| chr1 | 21221429 | 21221573 |
| chr1 | 21430532 | 21430654 |
| chr1 | 21516047 | 21516195 |
| chr1 | 21729069 | 21729190 |
| chr1 | 21910613 | 21910737 |
| chr1 | 21988753 | 21988919 |
| chr1 | 22076558 | 22076680 |
| chr1 | 22283757 | 22283885 |
| chr1 | 22426125 | 22426261 |
| chr1 | 22616943 | 22617073 |
| chr1 | 22684839 | 22684975 |
| chr1 | 22954292 | 22954426 |
| chr1 | 23017529 | 23017661 |
| chr1 | 23215094 | 23215220 |
| chr1 | 23277295 | 23277437 |
| chr1 | 23432278 | 23432434 |
| chr1 | 23622637 | 23622785 |
| chr1 | 23752872 | 23753004 |
| chr1 | 23991684 | 23991824 |
| chr1 | 24154476 | 24154614 |
| chr1 | 24286834 | 24287089 |
| chr1 | 24288599 | 24288723 |
| chr1 | 24340642 | 24340897 |
| chr1 | 24491953 | 24492087 |
| chr1 | 24641699 | 24641833 |
| chr1 | 24852344 | 24852466 |
| chr1 | 25020949 | 25021073 |
| chr1 | 25097356 | 25097480 |
| chr1 | 25326567 | 25326689 |
| chr1 | 25498984 | 25499106 |
| chr1 | 25580151 | 25580279 |
| chr1 | 25719561 | 25719687 |
| chr1 | 25887435 | 25887603 |
| chr1 | 26092204 | 26092330 |
| chr1 | 26156818 | 26156952 |
| chr1 | 26370177 | 26370299 |
| chr1 | 26475483 | 26475609 |
| chr1 | 26702954 | 26703088 |
| chr1 | 26743821 | 26743945 |
| chr1 | 27009336 | 27009458 |
| chr1 | 27033320 | 27033446 |
| chr1 | 27313716 | 27313915 |
| chr1 | 27412428 | 27412558 |
| chr1 | 27493641 | 27493765 |
| chr1 | 27773840 | 27773990 |
| chr1 | 27885840 | 27886086 |
| chr1 | 27971434 | 27971580 |
| chr1 | 28104441 | 28104723 |
| chr1 | 28236628 | 28236760 |
| chr1 | 28394123 | 28394265 |
| chr1 | 28589500 | 28589634 |
| chr1 | 28776559 | 28776705 |
| chr1 | 28894185 | 28894311 |
| chr1 | 29027586 | 29027716 |
| chr1 | 29238811 | 29238961 |
| chr1 | 29300280 | 29300404 |
| chr1 | 29428920 | 29429054 |
| chr1 | 29608266 | 29608410 |
| chr1 | 29830885 | 29831025 |
| chr1 | 30031764 | 30031885 |
| chr1 | 30191109 | 30191237 |
| chr1 | 30342292 | 30342416 |
| chr1 | 30496387 | 30496521 |
| chr1 | 30675791 | 30675933 |
| chr1 | 30777033 | 30777161 |
| chr1 | 31062544 | 31062668 |
| chr1 | 31164534 | 31164660 |
| chr1 | 31276508 | 31276650 |
| chr1 | 31485097 | 31485229 |
| chr1 | 31560305 | 31560445 |
| chr1 | 31741521 | 31741665 |
| chr1 | 31903598 | 31903722 |
| chr1 | 32018262 | 32018388 |
| chr1 | 32147589 | 32147727 |
| chr1 | 32302056 | 32302190 |
| chr1 | 32537771 | 32537971 |
| chr1 | 32616851 | 32617115 |
| chr1 | 32812041 | 32812298 |
| chr1 | 32887180 | 32887301 |
| chr1 | 33166839 | 33167111 |
| chr1 | 33289352 | 33289476 |
| chr1 | 33446789 | 33446913 |
| chr1 | 33583098 | 33583236 |
| chr1 | 33761608 | 33761732 |
| chr1 | 33802856 | 33802994 |
| chr1 | 34054772 | 34054896 |
| chr1 | 34215863 | 34215993 |
| chr1 | 34345171 | 34345333 |
| chr1 | 34515240 | 34515364 |
| chr1 | 34597836 | 34597962 |
| chr1 | 34809823 | 34809957 |
| chr1 | 34931590 | 34931764 |
| chr1 | 35119939 | 35120061 |
| chr1 | 35174645 | 35174781 |
| chr1 | 35397783 | 35397909 |
| chr1 | 35530683 | 35530927 |
| chr1 | 35687125 | 35687259 |
| chr1 | 35783116 | 35783392 |
| chr1 | 35939400 | 35939522 |
| chr1 | 36083641 | 36083762 |
| chr1 | 36195194 | 36195316 |
| chr1 | 36382392 | 36382534 |
| chr1 | 36603127 | 36603251 |
| chr1 | 36716889 | 36717090 |
| chr1 | 36912485 | 36912619 |
| chr1 | 37033074 | 37033216 |
| chr1 | 37114014 | 37114152 |
| chr1 | 37320877 | 37321005 |
| chr1 | 37430730 | 37430852 |
| chr1 | 37605351 | 37605489 |
| chr1 | 37711359 | 37711495 |
| chr1 | 37848757 | 37848881 |
| chr1 | 38109525 | 38109659 |
| chr1 | 38177282 | 38177408 |
| chr1 | 38309812 | 38309956 |
| chr1 | 38480027 | 38480163 |
| chr1 | 38619105 | 38619229 |
| chr1 | 38858460 | 38858588 |
| chr1 | 38937198 | 38937322 |
| chr1 | 39135410 | 39135554 |
| chr1 | 39258630 | 39258762 |
| chr1 | 39467762 | 39467908 |
| chr1 | 39507101 | 39507239 |
| chr1 | 39733697 | 39733821 |
| chr1 | 39820836 | 39820976 |
| chr1 | 39945207 | 39945329 |
| chr1 | 40136008 | 40136130 |
| chr1 | 40242877 | 40243003 |
| chr1 | 40395093 | 40395235 |
| chr1 | 40672332 | 40672494 |
| chr1 | 40806923 | 40807047 |
| chr1 | 40866200 | 40866350 |
| chr1 | 41021346 | 41021486 |
| chr1 | 41160130 | 41160252 |
| chr1 | 41397638 | 41397768 |
| chr1 | 41581984 | 41582106 |
| chr1 | 41849837 | 41849961 |
| chr1 | 41900630 | 41900792 |
| chr1 | 42105685 | 42105813 |
| chr1 | 42256269 | 42256405 |
| chr1 | 42334709 | 42334847 |
| chr1 | 42562858 | 42563006 |
| chr1 | 42725588 | 42725732 |
| chr1 | 42876339 | 42876481 |
| chr1 | 42941769 | 42941917 |
| chr1 | 43100154 | 43100282 |
| chr1 | 43277222 | 43277348 |
| chr1 | 43471108 | 43471236 |
| chr1 | 43623568 | 43623692 |
| chr1 | 43761563 | 43761687 |
| chr1 | 43857030 | 43857154 |
| chr1 | 43981209 | 43981341 |
| chr1 | 44240449 | 44240589 |
| chr1 | 44274641 | 44274783 |
| chr1 | 44468943 | 44469081 |
| chr1 | 44653797 | 44653931 |
| chr1 | 44801603 | 44801743 |
| chr1 | 44923431 | 44923561 |
| chr1 | 45096690 | 45096834 |
| chr1 | 45259205 | 45259375 |
| chr1 | 45430158 | 45430288 |
| chr1 | 45611672 | 45611797 |
| chr1 | 45720812 | 45720938 |
| chr1 | 45872572 | 45872726 |
| chr1 | 46013124 | 46013246 |
| chr1 | 46187268 | 46187400 |
| chr1 | 46309358 | 46309498 |
| chr1 | 46444773 | 46444901 |
| chr1 | 46528523 | 46528653 |
| chr1 | 46734704 | 46734830 |
| chr1 | 46895587 | 46895721 |
| chr1 | 47109842 | 47109970 |
| chr1 | 47213332 | 47213458 |
| chr1 | 47297751 | 47297872 |
| chr1 | 47525173 | 47525294 |
| chr1 | 47629978 | 47630101 |
| chr1 | 47789592 | 47789714 |
| chr1 | 47936642 | 47936778 |
| chr1 | 48161591 | 48161713 |
| chr1 | 48203902 | 48204044 |
| chr1 | 48442110 | 48442268 |
| chr1 | 48492019 | 48492145 |
| chr1 | 48647039 | 48647195 |
| chr1 | 48850333 | 48850467 |
| chr1 | 49019398 | 49019534 |
| chr1 | 49204654 | 49204802 |
| chr1 | 49303620 | 49303742 |
| chr1 | 49508193 | 49508317 |
| chr1 | 49615478 | 49615618 |
| chr1 | 49676702 | 49676846 |
| chr1 | 49857859 | 49857981 |
| chr1 | 50081515 | 50081649 |
| chr1 | 50191021 | 50191145 |
| chr1 | 50371964 | 50372086 |
| chr1 | 50509286 | 50509410 |
| chr1 | 50581405 | 50581529 |
| chr1 | 50871795 | 50871917 |
| chr1 | 50961505 | 50961626 |
| chr1 | 51093920 | 51094082 |
| chr1 | 51220037 | 51220177 |
| chr1 | 51382105 | 51382240 |
| chr1 | 51549338 | 51549460 |
| chr1 | 51734347 | 51734614 |
| chr1 | 51799056 | 51799190 |
| chr1 | 52061216 | 52061482 |
| chr1 | 52198147 | 52198269 |
| chr1 | 52354302 | 52354442 |
| chr1 | 52469540 | 52469711 |
| chr1 | 52568506 | 52568627 |
| chr1 | 52800968 | 52801261 |
| chr1 | 52913675 | 52913799 |
| chr1 | 53114252 | 53114400 |
| chr1 | 53244295 | 53244419 |
| chr1 | 53407821 | 53407977 |
| chr1 | 53515267 | 53515389 |
| chr1 | 53701503 | 53701643 |
| chr1 | 53752562 | 53752708 |
| chr1 | 53922103 | 53922241 |
| chr1 | 54172856 | 54173012 |
| chr1 | 54257322 | 54257462 |
| chr1 | 54470703 | 54470829 |
| chr1 | 54566220 | 54566362 |
| chr1 | 54684183 | 54684317 |
| chr1 | 54887947 | 54888079 |
| chr1 | 55025304 | 55025436 |
| chr1 | 55222327 | 55222449 |
| chr1 | 55248624 | 55248752 |
| chr1 | 55504598 | 55504724 |
| chr1 | 55666391 | 55666521 |
| chr1 | 55731183 | 55731305 |
| chr1 | 55944481 | 55944605 |
| chr1 | 56018690 | 56018812 |
| chr1 | 56139591 | 56139739 |
| chr1 | 56304467 | 56304589 |
| chr1 | 56519129 | 56519253 |
| chr1 | 56690277 | 56690401 |
| chr1 | 56836026 | 56836152 |
| chr1 | 57009388 | 57009510 |
| chr1 | 57057695 | 57057817 |
| chr1 | 57193017 | 57193151 |
| chr1 | 57432038 | 57432166 |
| chr1 | 57605340 | 57605488 |
| chr1 | 57742727 | 57742853 |
| chr1 | 57776344 | 57776486 |
| chr1 | 58002971 | 58003121 |
| chr1 | 58103589 | 58103721 |
| chr1 | 58296973 | 58297125 |
| chr1 | 58489395 | 58489529 |
| chr1 | 58643215 | 58643363 |
| chr1 | 58784301 | 58784427 |
| chr1 | 58830707 | 58830837 |
| chr1 | 59119180 | 59119310 |
| chr1 | 59241938 | 59242076 |
| chr1 | 59402510 | 59402634 |
| chr1 | 59478471 | 59478601 |
| chr1 | 59694551 | 59694693 |
| chr1 | 59830725 | 59830867 |
| chr1 | 59965703 | 59965861 |
| chr1 | 60069430 | 60069551 |
| chr1 | 60201105 | 60201243 |
| chr1 | 60327051 | 60327187 |
| chr1 | 60575837 | 60575981 |
| chr1 | 6071026 | 60710402 |
| chr1 | 60913987 | 60914117 |
| chr1 | 60961848 | 60961974 |
| chr1 | 61156643 | 61156783 |
| chr1 | 61257124 | 61257250 |
| chr1 | 61423099 | 61423263 |
| chr1 | 61556144 | 61556310 |
| chr1 | 61712576 | 61712697 |
| chr1 | 61913729 | 61913850 |
| chr1 | 62085334 | 62085462 |
| chr1 | 62210391 | 62210547 |
| chr1 | 62357709 | 62357859 |
| chr1 | 62482038 | 62482164 |
| chr1 | 62674782 | 62674916 |
| chr1 | 62859171 | 62859297 |
| chr1 | 62922608 | 62922730 |
| chr1 | 63156052 | 63156174 |
| chr1 | 63290054 | 63290182 |
| chr1 | 63338142 | 63338274 |
| chr1 | 63541030 | 63541174 |
| chr1 | 63701677 | 63701801 |
| chr1 | 63900687 | 63900825 |
| chr1 | 64027804 | 64027950 |
| chr1 | 64127378 | 64127506 |
| chr1 | 64300929 | 64301063 |
| chr1 | 64441287 | 64441411 |
| chr1 | 64596730 | 64596854 |
| chr1 | 64693865 | 64693989 |
| chr1 | 64870640 | 64870774 |
| chr1 | 65000707 | 65000839 |
| chr1 | 65146222 | 65146370 |
| chr1 | 65274985 | 65275125 |
| chr1 | 65542353 | 65542503 |
| chr1 | 65663324 | 65663460 |
| chr1 | 65836146 | 65836306 |
| chr1 | 65935420 | 65935542 |
| chr1 | 66124788 | 66124934 |
| chr1 | 66321127 | 66321269 |
| chr1 | 66362897 | 66363019 |
| chr1 | 66552619 | 66552757 |
| chr1 | 66646706 | 66646828 |
| chr1 | 66778219 | 66778345 |
| chr1 | 67005532 | 67005670 |
| chr1 | 67100804 | 67100932 |
| chr1 | 67237390 | 67237536 |
| chr1 | 67479546 | 67479686 |
| chr1 | 67571827 | 67571979 |
| chr1 | 67696495 | 67696625 |
| chr1 | 67912205 | 67912339 |
| chr1 | 68056076 | 68056230 |
| chr1 | 68135748 | 68135874 |
| chr1 | 68330994 | 68331134 |
| chr1 | 68507063 | 68507534 |
| chr1 | 68713311 | 68713435 |
| chr1 | 68864128 | 68864252 |
| chr1 | 69007305 | 69007429 |
| chr1 | 69093541 | 69093665 |
| chr1 | 69281665 | 69281791 |
| chr1 | 69358972 | 69359093 |
| chr1 | 69579200 | 69579322 |
| chr1 | 69651406 | 69651558 |
| chr1 | 69829849 | 69830098 |
| chr1 | 70021226 | 70021358 |
| chr1 | 70180388 | 70180518 |
| chr1 | 70262917 | 70263087 |
| chr1 | 70398982 | 70399106 |
| chr1 | 70583805 | 70583969 |
| chr1 | 70690460 | 70690588 |
| chr1 | 70903992 | 70904118 |
| chr1 | 71002574 | 71002716 |
| chr1 | 71178915 | 71179051 |
| chr1 | 71406509 | 71406645 |
| chr1 | 71480520 | 71480644 |
| chr1 | 71703358 | 71703488 |
| chr1 | 71830107 | 71830245 |
| chr1 | 71996050 | 71996176 |
| chr1 | 72080514 | 72080648 |
| chr1 | 72309554 | 72309700 |
| chr1 | 72426911 | 72427039 |
| chr1 | 72499874 | 72500000 |
| chr1 | 72707190 | 72707312 |
| chr1 | 72877855 | 72877977 |
| chr1 | 73071380 | 73071512 |
| chr1 | 73136008 | 73136154 |
| chr1 | 73263827 | 73263951 |
| chr1 | 73459699 | 73459831 |
| chr1 | 73625755 | 73625901 |
| chr1 | 73701685 | 73701809 |
| chr1 | 73899678 | 73899826 |
| chr1 | 74049302 | 74049426 |
| chr1 | 74188513 | 74188657 |
| chr1 | 74417668 | 74417798 |
| chr1 | 74501403 | 74501529 |
| chr1 | 74710364 | 74710498 |
| chr1 | 74842697 | 74842841 |
| chr1 | 75012585 | 75012715 |
| chr1 | 75089078 | 75089202 |
| chr1 | 75313401 | 75313533 |
| chr1 | 75426134 | 75426292 |
| chr1 | 75571631 | 75571761 |
| chr1 | 75717496 | 75717626 |
| chr1 | 75884086 | 75884216 |
| chr1 | 76053037 | 76053161 |
| chr1 | 76080126 | 76080250 |
| chr1 | 76263045 | 76263216 |
| chr1 | 76444034 | 76444160 |
| chr1 | 76548692 | 76548834 |
| chr1 | 76786032 | 76786172 |
| chr1 | 76890687 | 76890835 |
| chr1 | 77023189 | 77023321 |
| chr1 | 77154136 | 77154276 |
| chr1 | 77410355 | 77410495 |
| chr1 | 77446077 | 77446207 |
| chr1 | 77630353 | 77630507 |
| chr1 | 77809794 | 77809930 |
| chr1 | 77947165 | 77947293 |
| chr1 | 78133002 | 78133136 |
| chr1 | 78266357 | 78266489 |
| chr1 | 78419531 | 78419657 |
| chr1 | 78524120 | 78524250 |
| chr1 | 78688570 | 78688706 |
| chr1 | 78791718 | 78791856 |
| chr1 | 78967735 | 78967875 |
| chr1 | 79123012 | 79123144 |
| chr1 | 79330469 | 79330623 |
| chr1 | 79499114 | 79499248 |
| chr1 | 79664640 | 79664784 |
| chr1 | 79818983 | 79819129 |
| chr1 | 79960547 | 79960685 |
| chr1 | 80052417 | 80052549 |
| chr1 | 80234472 | 80234600 |
| chr1 | 80329228 | 80329368 |
| chr1 | 80511570 | 80511748 |
| chr1 | 80589894 | 80590038 |
| chr1 | 80833398 | 80833524 |
| chr1 | 80942067 | 80942201 |
| chr1 | 81136182 | 81136306 |
| chr1 | 81288637 | 81288777 |
| chr1 | 81364539 | 81364663 |
| chr1 | 81546858 | 81546982 |
| chr1 | 81661502 | 81661626 |
| chr1 | 81818240 | 81818378 |
| chr1 | 82049185 | 82049321 |
| chr1 | 82126490 | 82126614 |
| chr1 | 82296637 | 82296781 |
| chr1 | 82388824 | 82388950 |
| chr1 | 82627490 | 82627622 |
| chr1 | 82766833 | 82766971 |
| chr1 | 82874030 | 82874158 |
| chr1 | 83084819 | 83084949 |
| chr1 | 83160151 | 83160275 |
| chr1 | 83376832 | 83376958 |
| chr1 | 83454175 | 83454297 |
| chr1 | 83615421 | 83615567 |
| chr1 | 83762477 | 83762625 |
| chr1 | 83826631 | 83826755 |
| chr1 | 83919722 | 83919842 |
| chr1 | 83955633 | 83955791 |
| chr1 | 84044228 | 84044378 |
| chr1 | 84248277 | 84248405 |
| chr1 | 84398430 | 84398552 |
| chr1 | 84528344 | 84528468 |
| chr1 | 84662630 | 84662760 |
| chr1 | 84812310 | 84812439 |
| chr1 | 84934788 | 84934934 |
| chr1 | 85115778 | 85115916 |
| chr1 | 85350262 | 85350398 |
| chr1 | 85408980 | 85409114 |
| chr1 | 85649384 | 85649536 |
| chr1 | 85785699 | 85785823 |
| chr1 | 85967925 | 85968063 |
| chr1 | 86063563 | 86063687 |
| chr1 | 86190576 | 86190700 |
| chr1 | 86326491 | 86326629 |
| chr1 | 86459678 | 86459820 |
| chr1 | 86692061 | 86692203 |
| chr1 | 86802747 | 86802889 |
| chr1 | 86892332 | 86892476 |
| chr1 | 87106062 | 87106184 |
| chr1 | 87265716 | 87265840 |
| chr1 | 87328475 | 87328601 |
| chr1 | 87506081 | 87506209 |
| chr1 | 87655137 | 87655261 |
| chr1 | 87876327 | 87876451 |
| chr1 | 88033614 | 88033738 |
| chr1 | 88087936 | 88088076 |
| chr1 | 88334129 | 88334271 |
| chr1 | 88488290 | 88488450 |
| chr1 | 88540495 | 88540625 |
| chr1 | 88724761 | 88724889 |
| chr1 | 88960121 | 88960251 |
| chr1 | 88975320 | 88975456 |
| chr1 | 89222364 | 89222508 |
| chr1 | 89373321 | 89373455 |
| chr1 | 89438512 | 89438654 |
| chr1 | 89666874 | 89666996 |
| chr1 | 89745957 | 89746103 |
| chr1 | 89989282 | 89989406 |
| chr1 | 90025653 | 90025777 |
| chr1 | 90196474 | 90196600 |
| chr1 | 90343862 | 90343988 |
| chr1 | 90558302 | 90558426 |
| chr1 | 90693790 | 90693914 |
| chr1 | 90822653 | 90822779 |
| chr1 | 90979633 | 90979771 |
| chr1 | 91102606 | 91102732 |
| chr1 | 91336035 | 91336165 |
| chr1 | 91413874 | 91414140 |
| chr1 | 91566371 | 91566495 |
| chr1 | 91785495 | 91785645 |
| chr1 | 91961029 | 91961171 |
| chr1 | 92018341 | 92018473 |
| chr1 | 92213177 | 92213321 |
| chr1 | 92378759 | 92378883 |
| chr1 | 92447949 | 92448083 |
| chr1 | 92676816 | 92677070 |
| chr1 | 92727299 | 92727423 |
| chr1 | 93004451 | 93004577 |
| chr1 | 93065509 | 93065631 |
| chr1 | 93216930 | 93217060 |
| chr1 | 93458991 | 93459117 |
| chr1 | 93480143 | 93480271 |
| chr1 | 93666313 | 93666437 |
| chr1 | 93788682 | 93788828 |
| chr1 | 94002759 | 94002883 |
| chr1 | 94115235 | 94115363 |
| chr1 | 94292109 | 94292231 |
| chr1 | 94388479 | 94388623 |
| chr1 | 94535987 | 94536115 |
| chr1 | 94760645 | 94760771 |
| chr1 | 94971799 | 94971933 |
| chr1 | 95119750 | 95119878 |
| chr1 | 95157954 | 95158076 |
| chr1 | 95279399 | 95279545 |
| chr1 | 95572472 | 95572602 |
| chr1 | 95599459 | 95599585 |
| chr1 | 95850588 | 95850736 |
| chr1 | 95889861 | 95890005 |
| chr1 | 96072674 | 96072816 |
| chr1 | 96217479 | 96217607 |
| chr1 | 96414269 | 96414415 |
| chr1 | 96587745 | 96587883 |
| chr1 | 96694500 | 96694644 |
| chr1 | 96805944 | 96806074 |
| chr1 | 97007703 | 97007835 |
| chr1 | 97108291 | 97108441 |
| chr1 | 97249944 | 97250076 |
| chr1 | 97428328 | 97428466 |
| chr1 | 97621277 | 97621415 |
| chr1 | 97750494 | 97750624 |
| chr1 | 97904728 | 97904858 |
| chr1 | 98044891 | 98045142 |
| chr1 | 98208766 | 98208939 |
| chr1 | 98399507 | 98399689 |
| chr1 | 98537439 | 98537689 |
| chr1 | 98597124 | 98597258 |
| chr1 | 98855124 | 98855250 |
| chr1 | 99005630 | 99005756 |
| chr1 | 99081221 | 99081349 |
| chr1 | 99258219 | 99258341 |
| chr1 | 99328355 | 99328479 |
| chr1 | 99521230 | 99521364 |
| chr1 | 99648884 | 99649006 |
| chr1 | 99811401 | 99811523 |
| chr1 | 100015887 | 100016013 |
| chr1 | 100075596 | 100075728 |
| chr1 | 100245543 | 100245671 |
| chr1 | 100397080 | 100397216 |
| chr1 | 100641359 | 100641497 |
| chr1 | 100793991 | 100794145 |
| chr1 | 100885002 | 100885130 |
| chr1 | 101001571 | 101001711 |
| chr1 | 101239927 | 101240059 |
| chr1 | 101333665 | 101333825 |
| chr1 | 101564545 | 101564671 |
| chr1 | 101590263 | 101590395 |
| chr1 | 101836870 | 101837002 |
| chr1 | 101986380 | 101986514 |
| chr1 | 102099233 | 102099355 |
| chr1 | 102197465 | 102197641 |
| chr1 | 102404389 | 102404511 |
| chr1 | 102477046 | 102477186 |
| chr1 | 102735750 | 102735876 |
| chr1 | 102831321 | 102831447 |
| chr1 | 103062125 | 103062277 |
| chr1 | 103150405 | 103150551 |
| chr1 | 103367580 | 103367849 |
| chr1 | 103421392 | 103421583 |
| chr1 | 103552999 | 103553330 |
| chr1 | 103676756 | 103676880 |
| chr1 | 103916495 | 103916782 |
| chr1 | 104010228 | 104010349 |
| chr1 | 104125486 | 104125664 |
| chr1 | 104406419 | 104406541 |
| chr1 | 104536011 | 104536163 |
| chr1 | 104674070 | 104674214 |
| chr1 | 104735997 | 104736139 |
| chr1 | 104939399 | 104939529 |
| chr1 | 105082832 | 105082956 |
| chr1 | 105292262 | 105292396 |
| chr1 | 105394119 | 105394261 |
| chr1 | 105558126 | 105558266 |
| chr1 | 105736876 | 105737024 |
| chr1 | 105825473 | 105825613 |
| chr1 | 105962144 | 105962268 |
| chr1 | 106141576 | 106141712 |
| chr1 | 106286264 | 106286388 |
| chr1 | 106395078 | 106395212 |
| chr1 | 106640029 | 106640175 |
| chr1 | 106765171 | 106765297 |
| chr1 | 106830341 | 106830481 |
| chr1 | 107002045 | 107002167 |
| chr1 | 107223551 | 107223697 |
| chr1 | 107368280 | 107368422 |
| chr1 | 107510855 | 107510983 |
| chr1 | 107603248 | 107603394 |
| chr1 | 107872878 | 107873006 |
| chr1 | 107990158 | 107990284 |
| chr1 | 108123599 | 108123743 |
| chr1 | 108184184 | 108184308 |
| chr1 | 108467296 | 108467448 |
| chr1 | 108596971 | 108597093 |
| chr1 | 108673353 | 108673475 |
| chr1 | 108899425 | 108899546 |
| chr1 | 109015243 | 109015368 |
| chr1 | 109115995 | 109116127 |
| chr1 | 109303267 | 109303393 |
| chr1 | 109515600 | 109515732 |
| chr1 | 109583498 | 109583632 |
| chr1 | 109718631 | 109718771 |
| chr1 | 109835297 | 109835421 |
| chr1 | 110104105 | 110104227 |
| chr1 | 110144569 | 110144705 |
| chr1 | 110312951 | 110313075 |
| chr1 | 110573075 | 110573323 |
| chr1 | 110680249 | 110680371 |
| chr1 | 110866846 | 110866970 |
| chr1 | 110895929 | 110896075 |
| chr1 | 111065988 | 111066112 |
| chr1 | 111199149 | 111199287 |
| chr1 | 111392493 | 111392617 |
| chr1 | 111550392 | 111550514 |
| chr1 | 111658064 | 111658188 |
| chr1 | 111810090 | 111810246 |
| chr1 | 112000991 | 112001119 |
| chr1 | 112163456 | 112163596 |
| chr1 | 112289931 | 112290055 |
| chr1 | 112403243 | 112403365 |
| chr1 | 112569883 | 112570015 |
| chr1 | 112822929 | 112823069 |
| chr1 | 112961679 | 112961803 |
| chr1 | 113006714 | 113006866 |
| chr1 | 113163548 | 113163674 |
| chr1 | 113362003 | 113362153 |
| chr1 | 113564556 | 113564692 |
| chr1 | 113673332 | 113673453 |
| chr1 | 113822208 | 113822334 |
| chr1 | 113936932 | 113937062 |
| chr1 | 114048451 | 114048572 |
| chr1 | 114318665 | 114318809 |
| chr1 | 114426764 | 114426898 |
| chr1 | 114482436 | 114482572 |
| chr1 | 114752993 | 114753117 |
| chr1 | 114838096 | 114838220 |
| chr1 | 115073718 | 115073852 |
| chr1 | 115219031 | 115219153 |
| chr1 | 115324327 | 115324614 |
| chr1 | 115488232 | 115488356 |
| chr1 | 115606722 | 115606846 |
| chr1 | 115697506 | 115697628 |
| chr1 | 115970955 | 115971107 |
| chr1 | 116034248 | 116034402 |
| chr1 | 116238436 | 116238560 |
| chr1 | 116279362 | 116279484 |
| chr1 | 116449992 | 116450124 |
| chr1 | 116709596 | 116709724 |
| chr1 | 116752306 | 116752444 |
| chr1 | 116975166 | 116975430 |
| chr1 | 117126261 | 117126382 |
| chr1 | 117159248 | 117159378 |
| chr1 | 117214688 | 117214814 |
| chr1 | 117388837 | 117388981 |
| chr1 | 117540338 | 117540474 |
| chr1 | 117665493 | 117665617 |
| chr1 | 117883752 | 117883882 |
| chr1 | 117953104 | 117953230 |
| chr1 | 118208634 | 118208762 |
| chr1 | 118320350 | 118320474 |
| chr1 | 118510694 | 118510840 |
| chr1 | 118550370 | 118550492 |
| chr1 | 118678703 | 118678847 |
| chr1 | 118925575 | 118925715 |
| chr1 | 119017990 | 119018140 |
| chr1 | 119226824 | 119226968 |
| chr1 | 119369757 | 119369893 |
| chr1 | 119494207 | 119494333 |
| chr1 | 119577781 | 119577925 |
| chr1 | 119750922 | 119751058 |
| chr1 | 119926723 | 119926861 |
| chr1 | 120070456 | 120070582 |
| chr1 | 120216991 | 120217137 |
| chr1 | 120387870 | 120387994 |
| chr1 | 120501591 | 120501733 |
| chr1 | 120561157 | 120561283 |
| chr1 | 120601700 | 120601826 |
| chr1 | 120658884 | 120659008 |
| chr1 | 120839609 | 120839744 |
| chr1 | 120936229 | 120936363 |
| chr1 | 121107926 | 121108054 |
| chr1 | 121135217 | 121135337 |
| chr1 | 121137485 | 121137623 |
| chr1 | 121313234 | 121313360 |
| chr1 | 142585789 | 142585909 |
| chr1 | 142641810 | 142641954 |
| chr1 | 142867191 | 142867335 |
| chr1 | 143140605 | 143140749 |
| chr1 | 143450385 | 143450529 |
| chr1 | 143500646 | 143500770 |
| chr1 | 143744226 | 143744352 |
| chr1 | 143912384 | 143912518 |
| chr1 | 144005519 | 144005639 |
| chr1 | 144022533 | 144022691 |
| chr1 | 144051605 | 144051729 |
| chr1 | 144095133 | 144095261 |
| chr1 | 144340551 | 144340677 |
| chr1 | 144452425 | 144452549 |
| chr1 | 144520799 | 144520925 |
| chr1 | 144555939 | 144556061 |
| chr1 | 144590094 | 144590218 |
| chr1 | 144692690 | 144692830 |
| chr1 | 144828973 | 144829117 |
| chr1 | 144838204 | 144838326 |
| chr1 | 144967993 | 144968133 |
| chr1 | 144999305 | 144999426 |
| chr1 | 145160717 | 145160841 |
| chr1 | 145219610 | 145219734 |
| chr1 | 145260120 | 145260246 |
| chr1 | 145368873 | 145369017 |
| chr1 | 145499656 | 145499800 |
| chr1 | 145560388 | 145560510 |
| chr1 | 145718059 | 145718199 |
| chr1 | 145829410 | 145829536 |
| chr1 | 146013284 | 146013462 |
| chr1 | 146033831 | 146033975 |
| chr1 | 146108941 | 146109065 |
| chr1 | 146164214 | 146164356 |
| chr1 | 146214765 | 146214885 |
| chr1 | 146356514 | 146356638 |
| chr1 | 146564490 | 146564614 |
| chr1 | 146629862 | 146629998 |
| chr1 | 146729894 | 146730016 |
| chr1 | 146997169 | 146997299 |
| chr1 | 147138827 | 147138955 |
| chr1 | 147240791 | 147240941 |
| chr1 | 147377706 | 147377829 |
| chr1 | 147555061 | 147555239 |
| chr1 | 147575612 | 147575756 |
| chr1 | 147650774 | 147650898 |
| chr1 | 147706041 | 147706183 |
| chr1 | 147726432 | 147726553 |
| chr1 | 147825394 | 147825515 |
| chr1 | 147931749 | 147931875 |
| chr1 | 147999521 | 147999645 |
| chr1 | 148004214 | 148004358 |
| chr1 | 148193069 | 148193193 |
| chr1 | 148246835 | 148246959 |
| chr1 | 148251541 | 148251685 |
| chr1 | 148515553 | 148515675 |
| chr1 | 148549467 | 148549591 |
| chr1 | 148594829 | 148594973 |
| chr1 | 148599543 | 148599667 |
| chr1 | 148679720 | 148679874 |
| chr1 | 148680456 | 148680580 |
| chr1 | 148756873 | 148757017 |
| chr1 | 148761585 | 148761709 |
| chr1 | 148841403 | 148841557 |
| chr1 | 148842140 | 148842264 |
| chr1 | 148856017 | 148856155 |
| chr1 | 148858304 | 148858424 |
| chr1 | 148890630 | 148890760 |
| chr1 | 149005286 | 149005410 |
| chr1 | 149005993 | 149006147 |
| chr1 | 149050012 | 149050128 |
| chr1 | 149063100 | 149063264 |
| chr1 | 149239750 | 149239874 |
| chr1 | 149259388 | 149259512 |
| chr1 | 149282320 | 149282474 |
| chr1 | 149368874 | 149369008 |
| chr1 | 149576548 | 149576674 |
| chr1 | 149645005 | 149645129 |
| chr1 | 149667890 | 149668044 |
| chr1 | 149680134 | 149680268 |
| chr1 | 149753830 | 149753964 |
| chr1 | 149815193 | 149815314 |
| chr1 | 149923379 | 149923631 |
| chr1 | 150080111 | 150080239 |
| chr1 | 150237564 | 150237712 |
| chr1 | 150354592 | 150354732 |
| chr1 | 150552556 | 150552710 |
| chr1 | 150683335 | 150683471 |
| chr1 | 150809857 | 150809987 |
| chr1 | 150961164 | 150961302 |
| chr1 | 151186009 | 151186139 |
| chr1 | 151357295 | 151357421 |
| chr1 | 151477141 | 151477263 |
| chr1 | 151632594 | 151632716 |
| chr1 | 151722942 | 151723106 |
| chr1 | 151897955 | 151898087 |
| chr1 | 151996534 | 151996656 |
| chr1 | 152184624 | 152184770 |
| chr1 | 152399967 | 152400091 |
| chr1 | 152508559 | 152508703 |
| chr1 | 152708535 | 152708669 |
| chr1 | 152869673 | 152869799 |
| chr1 | 152947113 | 152947251 |
| chr1 | 153135856 | 153135998 |
| chr1 | 153214488 | 153214612 |
| chr1 | 153455275 | 153455409 |
| chr1 | 153613459 | 153613613 |
| chr1 | 153689937 | 153690061 |
| chr1 | 153901073 | 153901199 |
| chr1 | 154047455 | 154047578 |
| chr1 | 154093735 | 154093859 |
| chr1 | 154291884 | 154292006 |
| chr1 | 154380381 | 154380503 |
| chr1 | 154538510 | 154538638 |
| chr1 | 154701350 | 154701478 |
| chr1 | 154930399 | 154930670 |
| chr1 | 155115204 | 155115330 |
| chr1 | 155197422 | 155197546 |
| chr1 | 155274870 | 155275010 |
| chr1 | 155433890 | 155434024 |
| chr1 | 155646857 | 155646986 |
| chr1 | 155811814 | 155811993 |
| chr1 | 155907781 | 155907905 |
| chr1 | 156140870 | 156141002 |
| chr1 | 156275219 | 156275357 |
| chr1 | 156461617 | 156461741 |
| chr1 | 156540674 | 156540802 |
| chr1 | 156765891 | 156766023 |
| chr1 | 156804031 | 156804183 |
| chr1 | 157017151 | 157017291 |
| chr1 | 157206087 | 157206217 |
| chr1 | 157360538 | 157360688 |
| chr1 | 157483358 | 157483484 |
| chr1 | 157641593 | 157641741 |
| chr1 | 157694558 | 157694682 |
| chr1 | 157921323 | 157921461 |
| chr1 | 158049814 | 158049940 |
| chr1 | 158140115 | 158140279 |
| chr1 | 158403267 | 158403401 |
| chr1 | 158506642 | 158506776 |
| chr1 | 158616558 | 158616696 |
| chr1 | 158804593 | 158804727 |
| chr1 | 158930291 | 158930415 |
| chr1 | 159092587 | 159092715 |
| chr1 | 159273302 | 159273460 |
| chr1 | 159439825 | 159439999 |
| chr1 | 159585509 | 159585633 |
| chr1 | 159707977 | 159708107 |
| chr1 | 159834387 | 159834521 |
| chr1 | 159997119 | 159997249 |
| chr1 | 160152793 | 160152965 |
| chr1 | 160224620 | 160224752 |
| chr1 | 160438991 | 160439133 |
| chr1 | 160583280 | 160583418 |
| chr1 | 160680387 | 160680521 |
| chr1 | 160859501 | 160859637 |
| chr1 | 161014300 | 161014458 |
| chr1 | 161229569 | 161229707 |
| chr1 | 161388894 | 161389034 |
| chr1 | 161502179 | 161502300 |
| chr1 | 161659010 | 161659180 |
| chr1 | 161849106 | 161849236 |
| chr1 | 161932103 | 161932247 |
| chr1 | 162109319 | 162109447 |
| chr1 | 162279678 | 162279810 |
| chr1 | 162462466 | 162462612 |
| chr1 | 162603645 | 162603789 |
| chr1 | 162654733 | 162654855 |
| chr1 | 162837724 | 162837860 |
| chr1 | 163028429 | 163028551 |
| chr1 | 163148642 | 163148768 |
| chr1 | 163341471 | 163341601 |
| chr1 | 163422083 | 163422235 |
| chr1 | 163544520 | 163544660 |
| chr1 | 163739627 | 163739751 |
| chr1 | 163873734 | 163873858 |
| chr1 | 163992308 | 163992438 |
| chr1 | 164195225 | 164195355 |
| chr1 | 164293366 | 164293488 |
| chr1 | 164503150 | 164503282 |
| chr1 | 164619836 | 164619988 |
| chr1 | 164797760 | 164797886 |
| chr1 | 164936799 | 164936945 |
| chr1 | 165116757 | 165116895 |
| chr1 | 165240515 | 165240639 |
| chr1 | 165353035 | 165353185 |
| chr1 | 165622483 | 165622615 |
| chr1 | 165685147 | 165685289 |
| chr1 | 165853167 | 165853303 |
| chr1 | 165934642 | 165934776 |
| chr1 | 166151259 | 166151397 |
| chr1 | 166243222 | 166243360 |
| chr1 | 166438859 | 166438993 |
| chr1 | 166547464 | 166547628 |
| chr1 | 166711237 | 166711365 |
| chr1 | 166904105 | 166904231 |
| chr1 | 167066519 | 167066693 |
| chr1 | 167127314 | 167127438 |
| chr1 | 167389717 | 167389841 |
| chr1 | 167504094 | 167504230 |
| chr1 | 167594742 | 167594866 |
| chr1 | 167861781 | 167861919 |
| chr1 | 167976757 | 167976883 |
| chr1 | 168117826 | 168117952 |
| chr1 | 168304239 | 168304387 |
| chr1 | 168356951 | 168357099 |
| chr1 | 168487569 | 168487697 |
| chr1 | 168712083 | 168712205 |
| chr1 | 168833463 | 168833589 |
| chr1 | 168942749 | 168942903 |
| chr1 | 169143158 | 169143282 |
| chr1 | 169342566 | 169342720 |
| chr1 | 169462851 | 169462999 |
| chr1 | 169658435 | 169658557 |
| chr1 | 169772913 | 169773065 |
| chr1 | 169882985 | 169883111 |
| chr1 | 170085379 | 170085501 |
| chr1 | 170167875 | 170167999 |
| chr1 | 170302706 | 170302848 |
| chr1 | 170535554 | 170535682 |
| chr1 | 170692038 | 170692174 |
| chr1 | 170845693 | 170845815 |
| chr1 | 170958406 | 170958548 |
| chr1 | 171119120 | 171119252 |
| chr1 | 171187969 | 171188109 |
| chr1 | 171456704 | 171456836 |
| chr1 | 171559544 | 171559694 |
| chr1 | 171648879 | 171649003 |
| chr1 | 171919532 | 171919666 |
| chr1 | 172072577 | 172072723 |
| chr1 | 172191342 | 172191464 |
| chr1 | 172333194 | 172333318 |
| chr1 | 172419471 | 172419599 |
| chr1 | 172582307 | 172582431 |
| chr1 | 172796195 | 172796345 |
| chr1 | 172828394 | 172828568 |
| chr1 | 173097223 | 173097373 |
| chr1 | 173144929 | 173145069 |
| chr1 | 173326048 | 173326172 |
| chr1 | 173556338 | 173556464 |
| chr1 | 173694474 | 173694624 |
| chr1 | 173773620 | 173773744 |
| chr1 | 173926375 | 173926501 |
| chr1 | 174154040 | 174154263 |
| chr1 | 174291499 | 174291654 |
| chr1 | 174466297 | 174466439 |
| chr1 | 174543798 | 174543934 |
| chr1 | 174756680 | 174756879 |
| chr1 | 174880773 | 74880897 |
| chr1 | 175006890 | 175007028 |
| chr1 | 175115493 | 175115615 |
| chr1 | 175344904 | 175345082 |
| chr1 | 175378177 | 175378305 |
| chr1 | 175655486 | 175655612 |
| chr1 | 175756449 | 175756573 |
| chr1 | 175886471 | 175886595 |
| chr1 | 176001738 | 176001864 |
| chr1 | 176249465 | 176249603 |
| chr1 | 176287200 | 176287344 |
| chr1 | 176457192 | 176457318 |
| chr1 | 176605149 | 176605279 |
| chr1 | 176802730 | 176802854 |
| chr1 | 176976956 | 176977092 |
| chr1 | 177053984 | 177054124 |
| chr1 | 177211085 | 177211217 |
| chr1 | 177362900 | 177363024 |
| chr1 | 177601978 | 177602126 |
| chr1 | 177679602 | 177679726 |
| chr1 | 177873345 | 177873471 |
| chr1 | 177943722 | 177943876 |
| chr1 | 178142160 | 178142284 |
| chr1 | 178357823 | 178357963 |
| chr1 | 178476464 | 178476588 |
| chr1 | 178626779 | 178626921 |
| chr1 | 178784906 | 178785028 |
| chr1 | 178942944 | 178943098 |
| chr1 | 179035372 | 179035518 |
| chr1 | 179172766 | 179172910 |
| chr1 | 179394338 | 179394460 |
| chr1 | 179440257 | 179440397 |
| chr1 | 179709110 | 179709246 |
| chr1 | 179853022 | 179853162 |
| chr1 | 179964801 | 179964937 |
| chr1 | 180126077 | 180126219 |
| chr1 | 180260303 | 180260433 |
| chr1 | 180419406 | 180419532 |
| chr1 | 180494572 | 180494696 |
| chr1 | 180643728 | 180643975 |
| chr1 | 180800013 | 180800141 |
| chr1 | 181018747 | 181018875 |
| chr1 | 181213649 | 181213771 |
| chr1 | 181232311 | 181232437 |
| chr1 | 181421291 | 181421437 |
| chr1 | 181579149 | 181579273 |
| chr1 | 181674827 | 181674961 |
| chr1 | 181870143 | 181870265 |
| chr1 | 182064426 | 182064550 |
| chr1 | 182274123 | 182274251 |
| chr1 | 182346176 | 182346308 |
| chr1 | 182460905 | 182461029 |
| chr1 | 182714533 | 182714657 |
| chr1 | 182831729 | 182831885 |
| chr1 | 182917445 | 182917624 |
| chr1 | 183037613 | 183037749 |
| chr1 | 183313838 | 183313968 |
| chr1 | 183405799 | 183405941 |
| chr1 | 183564247 | 183564373 |
| chr1 | 183714069 | 183714209 |
| chr1 | 183797882 | 183798036 |
| chr1 | 184032339 | 184032489 |
| chr1 | 184160929 | 184161063 |
| chr1 | 184288403 | 184288547 |
| chr1 | 184386027 | 184386157 |
| chr1 | 184533938 | 184534068 |
| chr1 | 184728796 | 184728930 |
| chr1 | 184872137 | 184872295 |
| chr1 | 185065134 | 185065258 |
| chr1 | 185157610 | 185157766 |
| chr1 | 185381602 | 185381748 |
| chr1 | 185497476 | 185497598 |
| chr1 | 185621841 | 185621965 |
| chr1 | 185758781 | 185759041 |
| chr1 | 185924796 | 185925043 |
| chr1 | 186039376 | 186039512 |
| chr1 | 186189402 | 186189552 |
| chr1 | 186471215 | 186471343 |
| chr1 | 186508658 | 186508804 |
| chr1 | 186676143 | 186676263 |
| chr1 | 186879849 | 186879973 |
| chr1 | 186966574 | 186966732 |
| chr1 | 187089758 | 187089936 |
| chr1 | 187320354 | 187320484 |
| chr1 | 187399819 | 187399941 |
| chr1 | 187661970 | 187662100 |
| chr1 | 187685063 | 187685209 |
| chr1 | 187908182 | 187908330 |
| chr1 | 188114443 | 188114569 |
| chr1 | 188183005 | 188183131 |
| chr1 | 188289905 | 188290027 |
| chr1 | 188477713 | 188477837 |
| chr1 | 188611165 | 188611289 |
| chr1 | 188730330 | 188730452 |
| chr1 | 188983566 | 188983704 |
| chr1 | 189107956 | 189108078 |
| chr1 | 189240566 | 189240690 |
| chr1 | 189407463 | 189407597 |
| chr1 | 189562327 | 189562449 |
| chr1 | 189742067 | 189742219 |
| chr1 | 189924182 | 189924306 |
| chr1 | 189998836 | 189998984 |
| chr1 | 190108797 | 190108953 |
| chr1 | 190259883 | 190260021 |
| chr1 | 190460804 | 190460950 |
| chr1 | 190533621 | 190533777 |
| chr1 | 190813314 | 190813456 |
| chr1 | 190911517 | 190911655 |
| chr1 | 191059369 | 191059507 |
| chr1 | 191146152 | 191146276 |
| chr1 | 191306205 | 191306353 |
| chr1 | 191431109 | 191431241 |
| chr1 | 191614936 | 191615062 |
| chr1 | 191793221 | 191793345 |
| chr1 | 191934058 | 191934182 |
| chr1 | 192102704 | 192102842 |
| chr1 | 192213146 | 192213292 |
| chr1 | 192424079 | 192424217 |
| chr1 | 192586649 | 192586773 |
| chr1 | 192732268 | 192732438 |
| chr1 | 192906403 | 192906580 |
| chr1 | 193036862 | 193036986 |
| chr1 | 193161634 | 193161756 |
| chr1 | 193366206 | 193366494 |
| chr1 | 193456518 | 193456648 |
| chr1 | 193569696 | 193569818 |
| chr1 | 193769265 | 193769395 |
| chr1 | 193873259 | 193873385 |
| chr1 | 194090892 | 194091016 |
| chr1 | 194266850 | 194266978 |
| chr1 | 194357430 | 194357572 |
| chr1 | 194494726 | 194494852 |
| chr1 | 194590331 | 194590475 |
| chr1 | 194743453 | 194743577 |
| chr1 | 194907716 | 194907884 |
| chr1 | 195027740 | 195027864 |
| chr1 | 195273733 | 195273871 |
| chr1 | 195469619 | 195469791 |
| chr1 | 195567156 | 195567286 |
| chr1 | 195649194 | 195649318 |
| chr1 | 195859205 | 195859329 |
| chr1 | 195997894 | 195998026 |
| chr1 | 196111628 | 196111766 |
| chr1 | 196281712 | 196281856 |
| chr1 | 196397739 | 196397863 |
| chr1 | 196603068 | 196603195 |
| chr1 | 196709298 | 196709419 |
| chr1 | 196867789 | 196867910 |
| chr1 | 197054937 | 197055061 |
| chr1 | 197245796 | 197245920 |
| chr1 | 197305200 | 197305354 |
| chr1 | 197504280 | 197504406 |
| chr1 | 197701629 | 197701751 |
| chr1 | 197826686 | 197826818 |
| chr1 | 197970234 | 197970366 |
| chr1 | 198144894 | 198145028 |
| chr1 | 198174773 | 198174925 |
| chr1 | 198384908 | 198385036 |
| chr1 | 198532249 | 198532383 |
| chr1 | 198627600 | 198627722 |
| chr1 | 198807808 | 198807936 |
| chr1 | 199010651 | 199010803 |
| chr1 | 199087059 | 199087183 |
| chr1 | 199265675 | 199265835 |
| chr1 | 199520500 | 199520640 |
| chr1 | 199644452 | 199644580 |
| chr1 | 199819346 | 199819492 |
| chr1 | 199966482 | 199966606 |
| chr1 | 200055025 | 200055149 |
| chr1 | 200145457 | 200145579 |
| chr1 | 200379931 | 200380053 |
| chr1 | 200446829 | 200446957 |
| chr1 | 200693942 | 200694078 |
| chr1 | 200834028 | 200834168 |
| chr1 | 200920290 | 200920422 |
| chr1 | 201133718 | 201133852 |
| chr1 | 201193423 | 201193547 |
| chr1 | 201400941 | 201401077 |
| chr1 | 201588500 | 201588638 |
| chr1 | 201721874 | 201722016 |
| chr1 | 201899122 | 201899248 |
| chr1 | 201972003 | 201972129 |
| chr1 | 202080246 | 202080416 |
| chr1 | 202244021 | 202244143 |
| chr1 | 202461299 | 202461425 |
| chr1 | 202638052 | 202638176 |
| chr1 | 202737173 | 202737297 |
| chr1 | 202883305 | 202883449 |
| chr1 | 203106215 | 203106365 |
| chr1 | 203169301 | 203169459 |
| chr1 | 203336740 | 203336864 |
| chr1 | 203437644 | 203437790 |
| chr1 | 203707716 | 203707858 |
| chr1 | 203753254 | 203753430 |
| chr1 | 203945449 | 203945571 |
| chr1 | 204132564 | 204132688 |
| chr1 | 204193718 | 204193846 |
| chr1 | 204345853 | 204345999 |
| chr1 | 204541891 | 204542021 |
| chr1 | 204747224 | 204747356 |
| chr1 | 204803019 | 204803151 |
| chr1 | 204997056 | 204997184 |
| chr1 | 205164588 | 205164738 |
| chr1 | 205326759 | 205326911 |
| chr1 | 205381888 | 205382010 |
| chr1 | 205580238 | 205580366 |
| chr1 | 205683977 | 205684117 |
| chr1 | 205919845 | 205919967 |
| chr1 | 206139081 | 206139215 |
| chr1 | 206214278 | 206214430 |
| chr1 | 206329532 | 206329828 |
| chr1 | 206485246 | 206485404 |
| chr1 | 206514315 | 206514439 |
| chr1 | 206558139 | 206558267 |
| chr1 | 206712503 | 206712629 |
| chr1 | 206768317 | 206768459 |
| chr1 | 206962547 | 206962697 |
| chr1 | 207095665 | 207095811 |
| chr1 | 207240504 | 207240636 |
| chr1 | 207360003 | 207360137 |
| chr1 | 207587945 | 207588071 |
| chr1 | 207640601 | 207640761 |
| chr1 | 207903592 | 207903742 |
| chr1 | 207993609 | 207993735 |
| chr1 | 208142838 | 208142972 |
| chr1 | 208281933 | 208282087 |
| chr1 | 208513464 | 208513602 |
| chr1 | 208554137 | 208554271 |
| chr1 | 208777050 | 208777172 |
| chr1 | 208855333 | 208855473 |
| chr1 | 209037519 | 209037697 |
| chr1 | 209126826 | 209126990 |
| chr1 | 209336170 | 209336318 |
| chr1 | 209528288 | 209528420 |
| chr1 | 209699899 | 209700023 |
| chr1 | 209861455 | 209861597 |
| chr1 | 209968229 | 209968375 |
| chr1 | 210156712 | 210156852 |
| chr1 | 210194934 | 210195072 |
| chr1 | 210458615 | 210458739 |
| chr1 | 210592535 | 210592659 |
| chr1 | 210635576 | 210635750 |
| chr1 | 210810134 | 210810258 |
| chr1 | 210995800 | 210995954 |
| chr1 | 211168693 | 211168841 |
| chr1 | 211275245 | 211275373 |
| chr1 | 211415276 | 211415412 |
| chr1 | 211568943 | 211569095 |
| chr1 | 211771977 | 211772103 |
| chr1 | 211936148 | 211936270 |
| chr1 | 212026949 | 212027079 |
| chr1 | 212152121 | 212152243 |
| chr1 | 212324445 | 212324589 |
| chr1 | 212500369 | 212500511 |
| chr1 | 212715973 | 212716097 |
| chr1 | 212776288 | 212776416 |
| chr1 | 212996357 | 212996507 |
| chr1 | 213164003 | 213164131 |
| chr1 | 213210213 | 213210339 |
| chr1 | 213434339 | 213434475 |
| chr1 | 213529126 | 213529268 |
| chr1 | 213666853 | 213667025 |
| chr1 | 213854091 | 213854223 |
| chr1 | 214006510 | 214006640 |
| chr1 | 214197689 | 214197968 |
| chr1 | 214292089 | 214292213 |
| chr1 | 214514317 | 214514439 |
| chr1 | 214612731 | 214612893 |
| chr1 | 214747221 | 214747343 |
| chr1 | 214839213 | 214839335 |
| chr1 | 214988138 | 214988276 |
| chr1 | 215151011 | 215151133 |
| chr1 | 215401799 | 215401933 |
| chr1 | 215546853 | 215547120 |
| chr1 | 215592209 | 215592333 |
| chr1 | 215740068 | 215740189 |
| chr1 | 215949258 | 215949404 |
| chr1 | 216098914 | 216099038 |
| chr1 | 216268062 | 216268188 |
| chr1 | 216452756 | 216452896 |
| chr1 | 216560829 | 216560951 |
| chr1 | 216682392 | 216682534 |
| chr1 | 216809821 | 216809953 |
| chr1 | 217027960 | 217028094 |
| chr1 | 217096151 | 217096285 |
| chr1 | 217365913 | 217366037 |
| chr1 | 217417537 | 217417675 |
| chr1 | 217565040 | 217565184 |
| chr1 | 217714402 | 217714544 |
| chr1 | 217931903 | 217932025 |
| chr1 | 217988682 | 217988806 |
| chr1 | 218218743 | 218218871 |
| chr1 | 218313171 | 218313295 |
| chr1 | 218516258 | 218516394 |
| chr1 | 218670267 | 218670391 |
| chr1 | 218739408 | 218739530 |
| chr1 | 218968706 | 218968850 |
| chr1 | 219088034 | 219088174 |
| chr1 | 219225481 | 219225605 |
| chr1 | 219415428 | 219415552 |
| chr1 | 219581342 | 219581482 |
| chr1 | 219677472 | 219677616 |
| chr1 | 219811721 | 219811863 |
| chr1 | 220050390 | 220050514 |
| chr1 | 220155623 | 220155747 |
| chr1 | 220237596 | 220237717 |
| chr1 | 220448288 | 220448418 |
| chr1 | 220569884 | 220570028 |
| chr1 | 220796478 | 220796602 |
| chr1 | 220934528 | 220934652 |
| chr1 | 220989642 | 220989772 |
| chr1 | 221128252 | 221128382 |
| chr1 | 221396261 | 221396395 |
| chr1 | 221472327 | 221472457 |
| chr1 | 221714594 | 221714724 |
| chr1 | 221825051 | 221825177 |
| chr1 | 221991362 | 221991498 |
| chr1 | 222038971 | 222039121 |
| chr1 | 222212039 | 222212173 |
| chr1 | 222360190 | 222360332 |
| chr1 | 222484655 | 222484805 |
| chr1 | 222682937 | 222683067 |
| chr1 | 222762954 | 222763114 |
| chr1 | 222813655 | 222813781 |
| chr1 | 223028118 | 223028278 |
| chr1 | 223105217 | 223105343 |
| chr1 | 223295180 | 223295302 |
| chr1 | 223487650 | 223487774 |
| chr1 | 223539425 | 223539579 |
| chr1 | 223724990 | 223725111 |
| chr1 | 223906023 | 223906161 |
| chr1 | 224075393 | 224075537 |
| chr1 | 224169876 | 224170006 |
| chr1 | 224176348 | 224176470 |
| chr1 | 224229479 | 224229705 |
| chr1 | 224411558 | 224411684 |
| chr1 | 224533819 | 224533985 |
| chr1 | 224647156 | 224647278 |
| chr1 | 224772577 | 224772723 |
| chr1 | 225005229 | 225005353 |
| chr1 | 225067241 | 225067383 |
| chr1 | 225182759 | 225182883 |
| chr1 | 225354331 | 225354485 |
| chr1 | 225520132 | 225520272 |
| chr1 | 225744283 | 225744407 |
| chr1 | 225842874 | 225843014 |
| chr1 | 225944989 | 225945139 |
| chr1 | 226165939 | 226166069 |
| chr1 | 226347381 | 226347521 |
| chr1 | 226459517 | 226459663 |
| chr1 | 226664686 | 226664828 |
| chr1 | 226721412 | 226721564 |
| chr1 | 226947388 | 226947546 |
| chr1 | 227047211 | 227047337 |
| chr1 | 227154197 | 227154321 |
| chr1 | 227408989 | 227409113 |
| chr1 | 227459447 | 227459579 |
| chr1 | 227636281 | 227636415 |
| chr1 | 227854103 | 227854227 |
| chr1 | 227994120 | 227994242 |
| chr1 | 228126430 | 228126554 |
| chr1 | 228244734 | 228244908 |
| chr1 | 228371664 | 228371794 |
| chr1 | 228546188 | 228546330 |
| chr1 | 228631637 | 228631775 |
| chr1 | 228924245 | 228924389 |
| chr1 | 229018876 | 229019002 |
| chr1 | 229195109 | 229195235 |
| chr1 | 229248884 | 229249018 |
| chr1 | 229422650 | 229422778 |
| chr1 | 229673850 | 229673976 |
| chr1 | 229710367 | 229710505 |
| chr1 | 229845721 | 229845885 |
| chr1 | 230088826 | 230089091 |
| chr1 | 230134642 | 230134794 |
| chr1 | 230388937 | 230389073 |
| chr1 | 230430328 | 230430454 |
| chr1 | 230681207 | 230681333 |
| chr1 | 230761147 | 230761289 |
| chr1 | 230948220 | 230948346 |
| chr1 | 231162439 | 231162565 |
| chr1 | 231311809 | 231311955 |
| chr1 | 231348942 | 231349074 |
| chr1 | 231614003 | 231614127 |
| chr1 | 231769372 | 231769494 |
| chr1 | 231879540 | 231879680 |
| chr1 | 231928899 | 231929023 |
| chr1 | 232161857 | 232161981 |
| chr1 | 232331909 | 232332051 |
| chr1 | 232423805 | 232423933 |
| chr1 | 232562678 | 232562802 |
| chr1 | 232795015 | 232795167 |
| chr1 | 232923787 | 232923911 |
| chr1 | 233090133 | 233090279 |
| chr1 | 233182227 | 233182365 |
| chr1 | 233341505 | 233341651 |
| chr1 | 233520084 | 233520212 |
| chr1 | 233601123 | 233601267 |
| chr1 | 233786218 | 233786348 |
| chr1 | 233876547 | 233876675 |
| chr1 | 234119748 | 234119872 |
| chr1 | 234206535 | 234206679 |
| chr1 | 234420691 | 234420831 |
| chr1 | 234493602 | 234493734 |
| chr1 | 234627915 | 234628043 |
| chr1 | 234781819 | 234781969 |
| chr1 | 235017895 | 235018037 |
| chr1 | 235178496 | 235178640 |
| chr1 | 235315593 | 235315727 |
| chr1 | 235432161 | 235432311 |
| chr1 | 235533349 | 235533483 |
| chr1 | 235781482 | 235781618 |
| chr1 | 235871887 | 235872011 |
| chr1 | 236111773 | 236111929 |
| chr1 | 236150520 | 236150650 |
| chr1 | 236309934 | 236310058 |
| chr1 | 236529049 | 236529185 |
| chr1 | 236636101 | 236636229 |
| chr1 | 236818693 | 236818817 |
| chr1 | 237022037 | 237022159 |
| chr1 | 237093530 | 237093668 |
| chr1 | 237199689 | 237199835 |
| chr1 | 237426783 | 237426915 |
| chr1 | 237589547 | 237589689 |
| chr1 | 237740219 | 237740351 |
| chr1 | 237886092 | 237886226 |
| chr1 | 238015433 | 238015579 |
| chr1 | 238211671 | 238211956 |
| chr1 | 238305452 | 238305578 |
| chr1 | 238403203 | 238403373 |
| chr1 | 238625820 | 238625964 |
| chr1 | 238706858 | 238706986 |
| chr1 | 238909929 | 238910053 |
| chr1 | 239048196 | 239048330 |
| chr1 | 239255583 | 239255731 |
| chr1 | 239378923 | 239379099 |
| chr1 | 239535325 | 239535469 |
| chr1 | 239581218 | 239581350 |
| chr1 | 239870685 | 239870827 |
| chr1 | 240018229 | 240018361 |
| chr1 | 240137721 | 240137853 |
| chr1 | 240305299 | 240305437 |
| chr1 | 240359600 | 240359722 |
| chr1 | 240614198 | 240614346 |
| chr1 | 240675163 | 240675287 |
| chr1 | 240869758 | 240869912 |
| chr1 | 241070319 | 241070441 |
| chr1 | 241177547 | 241177689 |
| chr1 | 241338623 | 241338747 |
| chr1 | 241439588 | 241439726 |
| chr1 | 241536526 | 241536672 |
| chr1 | 241720517 | 241720641 |
| chr1 | 241882613 | 241882735 |
| chr1 | 242062726 | 242062852 |
| chr1 | 242246753 | 242246891 |
| chr1 | 242292190 | 242292328 |
| chr1 | 242445226 | 242445362 |
| chr1 | 242618888 | 242619012 |
| chr1 | 242827272 | 242827402 |
| chr1 | 242924936 | 242925060 |
| chr1 | 243121126 | 243121272 |
| chr1 | 243250428 | 243250558 |
| chr1 | 243261089 | 243261211 |
| chr1 | 243381084 | 243381206 |
| chr1 | 243615505 | 243615653 |
| chr1 | 243731804 | 243731926 |
| chr1 | 243868310 | 243868450 |
| chr1 | 244024178 | 244024306 |
| chr1 | 244125636 | 244125760 |
| chr1 | 244297942 | 244298080 |
| chr1 | 244401661 | 244401799 |
| chr1 | 244609018 | 244609150 |
| chr1 | 244775807 | 244775953 |
| chr1 | 244974244 | 244974540 |
| chr1 | 245075269 | 245075409 |
| chr1 | 245148678 | 245148808 |
| chr1 | 245413418 | 245413552 |
| chr1 | 245470070 | 245470218 |
| chr1 | 245696274 | 245696398 |
| chr1 | 245807929 | 245808053 |
| chr1 | 246011243 | 246011391 |
| chr1 | 246101107 | 246101233 |
| chr1 | 246274999 | 246275159 |
| chr1 | 246341133 | 246341255 |
| chr1 | 246578679 | 246578807 |
| chr1 | 246717319 | 246717453 |
| chr1 | 246776393 | 246776529 |
| chr1 | 246949777 | 246949901 |
| chr1 | 247205596 | 247205744 |
| chr1 | 247259487 | 247259613 |
| chr1 | 247438394 | 247438530 |
| chr1 | 247624907 | 247625033 |
| chr1 | 247683065 | 247683211 |
| chr1 | 247857318 | 247857442 |
| chr1 | 248094530 | 248094682 |
| chr1 | 248227113 | 248227249 |
| chr1 | 248358604 | 248358728 |
| chr1 | 248497714 | 248497838 |
| chr1 | 248687367 | 248687488 |
| chr1 | 248837661 | 248837789 |
| chr1 | 248907414 | 248907568 |
| chr1 | 249128656 | 249128794 |
| chr1 | 249174477 | 249174770 |
| chr2 | 83962 | 84092 |
| chr2 | 298203 | 298355 |
| chr2 | 424089 | 424213 |
| chr2 | 613995 | 614119 |
| chr2 | 651547 | 651687 |
| chr2 | 830701 | 830849 |
| chr2 | 1007535 | 1007663 |
| chr2 | 1170847 | 1170973 |
| chr2 | 1327630 | 1327754 |
| chr2 | 1417154 | 1417300 |
| chr2 | 1618577 | 1618725 |
| chr2 | 1737070 | 1737212 |
| chr2 | 1860493 | 1860615 |
| chr2 | 2066621 | 2066747 |
| chr2 | 2209370 | 2209494 |
| chr2 | 2412830 | 2412960 |
| chr2 | 2545585 | 2545713 |
| chr2 | 2637687 | 2637823 |
| chr2 | 2858714 | 2858846 |
| chr2 | 2965542 | 2965664 |
| chr2 | 3093774 | 3093896 |
| chr2 | 3245499 | 3245635 |
| chr2 | 3437459 | 3437625 |
| chr2 | 3507879 | 3508009 |
| chr2 | 3658594 | 3658738 |
| chr2 | 3806960 | 3807104 |
| chr2 | 4057009 | 4057147 |
| chr2 | 4200337 | 4200463 |
| chr2 | 4317421 | 4317547 |
| chr2 | 4493518 | 4493664 |
| chr2 | 4626437 | 4626579 |
| chr2 | 4818584 | 4818718 |
| chr2 | 4876443 | 4876573 |
| chr2 | 4987388 | 4987516 |
| chr2 | 5240979 | 5241115 |
| chr2 | 5345684 | 5345820 |
| chr2 | 5516730 | 5516872 |
| chr2 | 5680487 | 5680617 |
| chr2 | 5748236 | 5748380 |
| chr2 | 5786376 | 5786498 |
| chr2 | 5988627 | 5988761 |
| chr2 | 6124873 | 6124997 |
| chr2 | 6233228 | 6233358 |
| chr2 | 6464945 | 6465071 |
| chr2 | 6616103 | 6616227 |
| chr2 | 6699162 | 6699284 |
| chr2 | 6865626 | 6865766 |
| chr2 | 6965340 | 6965464 |
| chr2 | 7108176 | 7108300 |
| chr2 | 7358061 | 7358203 |
| chr2 | 7512676 | 7512816 |
| chr2 | 7590097 | 7590233 |
| chr2 | 7767054 | 7767188 |
| chr2 | 7915754 | 7915882 |
| chr2 | 8011212 | 8011358 |
| chr2 | 8189856 | 8189992 |
| chr2 | 8423005 | 8423139 |
| chr2 | 8479865 | 8479989 |
| chr2 | 8618752 | 8618924 |
| chr2 | 8842511 | 8842651 |
| chr2 | 8929131 | 8929283 |
| chr2 | 9137155 | 9137400 |
| chr2 | 9312115 | 9312237 |
| chr2 | 9415034 | 9415160 |
| chr2 | 9515471 | 9515601 |
| chr2 | 9761301 | 9761429 |
| chr2 | 9920649 | 9920789 |
| chr2 | 9971494 | 9971650 |
| chr2 | 10090174 | 10090300 |
| chr2 | 10369935 | 10370057 |
| chr2 | 10520905 | 10521043 |
| chr2 | 10562054 | 10562192 |
| chr2 | 10732980 | 10733104 |
| chr2 | 10963532 | 10963658 |
| chr2 | 11097475 | 11097605 |
| chr2 | 11131576 | 11131698 |
| chr2 | 11340108 | 11340246 |
| chr2 | 11472481 | 11472613 |
| chr2 | 11692196 | 11692322 |
| chr2 | 11761368 | 11761492 |
| chr2 | 11947113 | 11947277 |
| chr2 | 12057470 | 12057594 |
| chr2 | 12204572 | 12204696 |
| chr2 | 12329609 | 12329731 |
| chr2 | 12596466 | 12596598 |
| chr2 | 12763205 | 12763331 |
| chr2 | 12787823 | 12787947 |
| chr2 | 13043810 | 13043940 |
| chr2 | 13186333 | 13186463 |
| chr2 | 13302194 | 13302318 |
| chr2 | 13492195 | 13492331 |
| chr2 | 13556487 | 13556607 |
| chr2 | 13610274 | 13610398 |
| chr2 | 13814522 | 13814666 |
| chr2 | 13973921 | 13974045 |
| chr2 | 14016248 | 14016392 |
| chr2 | 14154514 | 14154636 |
| chr2 | 14402101 | 14402223 |
| chr2 | 14546759 | 14546883 |
| chr2 | 14701108 | 14701232 |
| chr2 | 14790217 | 14790341 |
| chr2 | 14879777 | 14879899 |
| chr2 | 15134166 | 15134288 |
| chr2 | 15297213 | 15297337 |
| chr2 | 15357139 | 15357261 |
| chr2 | 15541118 | 15541242 |
| chr2 | 15676072 | 15676202 |
| chr2 | 15780145 | 15780279 |
| chr2 | 15953335 | 15953467 |
| chr2 | 16133787 | 16133911 |
| chr2 | 16334322 | 16334447 |
| chr2 | 16433828 | 16433962 |
| chr2 | 16569150 | 16569272 |
| chr2 | 16680749 | 16680871 |
| chr2 | 16896720 | 16896842 |
| chr2 | 17002567 | 17002691 |
| chr2 | 17229173 | 17229319 |
| chr2 | 17378603 | 17378731 |
| chr2 | 17444471 | 17444609 |
| chr2 | 17722592 | 17722724 |
| chr2 | 17841568 | 17841726 |
| chr2 | 18023357 | 18023501 |
| chr2 | 18099742 | 18099866 |
| chr2 | 18318548 | 18318688 |
| chr2 | 18368491 | 18368639 |
| chr2 | 18586746 | 18586884 |
| chr2 | 18671759 | 18671883 |
| chr2 | 18868668 | 18868792 |
| chr2 | 18947277 | 18947401 |
| chr2 | 19104091 | 19104219 |
| chr2 | 19298325 | 19298451 |
| chr2 | 19515049 | 19515175 |
| chr2 | 19619058 | 19619184 |
| chr2 | 19792719 | 19792865 |
| chr2 | 19885871 | 19885995 |
| chr2 | 20101840 | 20101964 |
| chr2 | 20159265 | 20159393 |
| chr2 | 20384512 | 20384636 |
| chr2 | 20503374 | 20503524 |
| chr2 | 20630220 | 20630344 |
| chr2 | 20861149 | 20861293 |
| chr2 | 20926480 | 20926606 |
| chr2 | 21101292 | 21101416 |
| chr2 | 21245251 | 21245377 |
| chr2 | 21429146 | 21429268 |
| chr2 | 21556771 | 21556895 |
| chr2 | 21634738 | 21634864 |
| chr2 | 21817307 | 21817568 |
| chr2 | 21930235 | 21930419 |
| chr2 | 22155098 | 22155248 |
| chr2 | 22277342 | 22277463 |
| chr2 | 22465951 | 22466077 |
| chr2 | 22664992 | 22665132 |
| chr2 | 22727950 | 22728090 |
| chr2 | 22878809 | 22878935 |
| chr2 | 23058841 | 23058965 |
| chr2 | 23174114 | 23174254 |
| chr2 | 23339839 | 23339965 |
| chr2 | 23446602 | 23446734 |
| chr2 | 23584475 | 23584599 |
| chr2 | 23743673 | 23743813 |
| chr2 | 23970630 | 23970768 |
| chr2 | 24164047 | 24164189 |
| chr2 | 24293305 | 24293445 |
| chr2 | 24476058 | 24476200 |
| chr2 | 24542119 | 24542389 |
| chr2 | 24643217 | 24643339 |
| chr2 | 24854555 | 24854693 |
| chr2 | 25029565 | 25029689 |
| chr2 | 25116887 | 25117015 |
| chr2 | 25242186 | 25242324 |
| chr2 | 25380535 | 25380659 |
| chr2 | 25677943 | 25678085 |
| chr2 | 25781385 | 25781525 |
| chr2 | 25895616 | 25895742 |
| chr2 | 25981125 | 25981291 |
| chr2 | 26195971 | 26196109 |
| chr2 | 26301230 | 26301370 |
| chr2 | 26462861 | 26463001 |
| chr2 | 26715433 | 26715569 |
| chr2 | 26792060 | 26792184 |
| chr2 | 26931989 | 26932115 |
| chr2 | 27115214 | 27115358 |
| chr2 | 27233773 | 27233907 |
| chr2 | 27470935 | 27471059 |
| chr2 | 27519168 | 27519302 |
| chr2 | 27631127 | 27631263 |
| chr2 | 27857463 | 27857587 |
| chr2 | 28023194 | 28023340 |
| chr2 | 28181950 | 28182082 |
| chr2 | 28271397 | 28271523 |
| chr2 | 28447196 | 28447460 |
| chr2 | 28602821 | 28602947 |
| chr2 | 28790653 | 28790799 |
| chr2 | 28939566 | 28939714 |
| chr2 | 28988047 | 28988169 |
| chr2 | 29163970 | 29164094 |
| chr2 | 29421431 | 29421581 |
| chr2 | 29559441 | 29559579 |
| chr2 | 29636964 | 29637088 |
| chr2 | 29734441 | 29734565 |
| chr2 | 29967808 | 29967956 |
| chr2 | 30042119 | 30042259 |
| chr2 | 30279420 | 30279566 |
| chr2 | 30437602 | 30437724 |
| chr2 | 30586228 | 30586354 |
| chr2 | 30686885 | 30687009 |
| chr2 | 30805214 | 30805336 |
| chr2 | 31061724 | 31061850 |
| chr2 | 31148976 | 31149100 |
| chr2 | 31319082 | 31319232 |
| chr2 | 31380722 | 31380848 |
| chr2 | 31559009 | 31559133 |
| chr2 | 31749264 | 31749666 |
| chr2 | 31865574 | 31865706 |
| chr2 | 32019707 | 32019835 |
| chr2 | 32230883 | 32231083 |
| chr2 | 32343131 | 32343253 |
| chr2 | 32502439 | 32502581 |
| chr2 | 32612058 | 32612300 |
| chr2 | 32835628 | 32835752 |
| chr2 | 33017944 | 33018076 |
| chr2 | 33110279 | 33110423 |
| chr2 | 33216751 | 33216883 |
| chr2 | 33461319 | 33461461 |
| chr2 | 33516068 | 33516200 |
| chr2 | 33695013 | 33695143 |
| chr2 | 33913953 | 33914077 |
| chr2 | 33955327 | 33955457 |
| chr2 | 34175899 | 34176025 |
| chr2 | 34360240 | 34360378 |
| chr2 | 34523149 | 34523283 |
| chr2 | 34661873 | 34661997 |
| chr2 | 34814050 | 34814194 |
| chr2 | 34912774 | 34912916 |
| chr2 | 35008574 | 35008696 |
| chr2 | 35130288 | 35130430 |
| chr2 | 35322530 | 35322654 |
| chr2 | 35487632 | 35487764 |
| chr2 | 35670617 | 35670739 |
| chr2 | 35794840 | 35794964 |
| chr2 | 35910167 | 35910329 |
| chr2 | 36157124 | 36157246 |
| chr2 | 36258890 | 36259014 |
| chr2 | 36399450 | 36399588 |
| chr2 | 36548903 | 36549049 |
| chr2 | 36750746 | 36750882 |
| chr2 | 36825638 | 36825766 |
| chr2 | 36984126 | 36984250 |
| chr2 | 37117037 | 37117161 |
| chr2 | 37342686 | 37342966 |
| chr2 | 37394702 | 37394824 |
| chr2 | 37642915 | 37643039 |
| chr2 | 37734055 | 37734189 |
| chr2 | 37829941 | 37830063 |
| chr2 | 38026090 | 38026214 |
| chr2 | 38191785 | 38191927 |
| chr2 | 38291931 | 38292055 |
| chr2 | 38571183 | 38571343 |
| chr2 | 38646780 | 38646928 |
| chr2 | 38878245 | 38878379 |
| chr2 | 38893895 | 38894025 |
| chr2 | 39113572 | 39113696 |
| chr2 | 39202801 | 39202922 |
| chr2 | 39341084 | 39341208 |
| chr2 | 39521774 | 39521898 |
| chr2 | 39687955 | 39688077 |
| chr2 | 39829297 | 39829421 |
| chr2 | 40000962 | 40001088 |
| chr2 | 40167919 | 40168057 |
| chr2 | 40305865 | 40305989 |
| chr2 | 40476065 | 40476205 |
| chr2 | 40590870 | 40591018 |
| chr2 | 40717155 | 40717297 |
| chr2 | 40932377 | 40932505 |
| chr2 | 41075064 | 41075206 |
| chr2 | 41157326 | 41157464 |
| chr2 | 41287066 | 41287226 |
| chr2 | 41497290 | 41497422 |
| chr2 | 41628425 | 41628551 |
| chr2 | 41773262 | 41773412 |
| chr2 | 41959583 | 41959727 |
| chr2 | 42043089 | 42043241 |
| chr2 | 42249832 | 42249976 |
| chr2 | 42355637 | 42355777 |
| chr2 | 42585775 | 42585921 |
| chr2 | 42737644 | 42737772 |
| chr2 | 42876794 | 42876938 |
| chr2 | 42935469 | 42935611 |
| chr2 | 43169236 | 43169358 |
| chr2 | 43372554 | 43372676 |
| chr2 | 43411754 | 43411906 |
| chr2 | 43599168 | 43599310 |
| chr2 | 43721426 | 43721574 |
| chr2 | 43870416 | 43870538 |
| chr2 | 43999668 | 43999822 |
| chr2 | 44245248 | 44245372 |
| chr2 | 44348700 | 44348826 |
| chr2 | 44536510 | 44536632 |
| chr2 | 44702926 | 44703054 |
| chr2 | 44831954 | 44832082 |
| chr2 | 44883706 | 44883850 |
| chr2 | 45117679 | 45117805 |
| chr2 | 45311573 | 45311713 |
| chr2 | 45434320 | 45434452 |
| chr2 | 45615838 | 45615960 |
| chr2 | 45681351 | 45681473 |
| chr2 | 45876615 | 45876739 |
| chr2 | 45994532 | 45994656 |
| chr2 | 46158020 | 46158144 |
| chr2 | 46267190 | 46267322 |
| chr2 | 46511924 | 46512048 |
| chr2 | 46582292 | 46582438 |
| chr2 | 46698371 | 46698515 |
| chr2 | 46891081 | 46891203 |
| chr2 | 47054939 | 47055063 |
| chr2 | 47266744 | 47266870 |
| chr2 | 47308665 | 47308797 |
| chr2 | 47522898 | 47523044 |
| chr2 | 47612790 | 47612912 |
| chr2 | 47854612 | 47854748 |
| chr2 | 47907438 | 47907564 |
| chr2 | 48174077 | 48174207 |
| chr2 | 48233960 | 48234098 |
| chr2 | 48468953 | 48469081 |
| chr2 | 48584242 | 48584374 |
| chr2 | 48649651 | 48649777 |
| chr2 | 48798669 | 48798791 |
| chr2 | 48998119 | 48998251 |
| chr2 | 49081154 | 49081278 |
| chr2 | 49296330 | 49296484 |
| chr2 | 49379936 | 49380066 |
| chr2 | 49676292 | 49676416 |
| chr2 | 49828743 | 49828883 |
| chr2 | 49885107 | 49885263 |
| chr2 | 50044034 | 50044158 |
| chr2 | 50232854 | 50232982 |
| chr2 | 50296804 | 50296930 |
| chr2 | 50440138 | 50440260 |
| chr2 | 50635393 | 50635523 |
| chr2 | 50821502 | 50821627 |
| chr2 | 50905686 | 50905832 |
| chr2 | 51031812 | 51031933 |
| chr2 | 51247256 | 51247378 |
| chr2 | 51458508 | 51458658 |
| chr2 | 51534345 | 51534469 |
| chr2 | 51736940 | 51737080 |
| chr2 | 51881337 | 51881481 |
| chr2 | 51946103 | 51946233 |
| chr2 | 52093985 | 52094117 |
| chr2 | 52347797 | 52347923 |
| chr2 | 52394884 | 52395014 |
| chr2 | 52536013 | 52536149 |
| chr2 | 52737133 | 52737263 |
| chr2 | 52858212 | 52858340 |
| chr2 | 53046795 | 53046921 |
| chr2 | 53174920 | 53175044 |
| chr2 | 53423597 | 53423729 |
| chr2 | 53489100 | 53489234 |
| chr2 | 53642245 | 53642371 |
| chr2 | 53799445 | 53799573 |
| chr2 | 54015921 | 54016063 |
| chr2 | 54075356 | 54075482 |
| chr2 | 54260990 | 54261118 |
| chr2 | 54354977 | 54355105 |
| chr2 | 54488055 | 54488185 |
| chr2 | 54687177 | 54687319 |
| chr2 | 54865489 | 54865611 |
| chr2 | 55051787 | 55051915 |
| chr2 | 55203950 | 55204076 |
| chr2 | 55346410 | 55346548 |
| chr2 | 55463486 | 55463628 |
| chr2 | 55649127 | 55649259 |
| chr2 | 55757831 | 55757973 |
| chr2 | 55931095 | 55931247 |
| chr2 | 56105860 | 56105982 |
| chr2 | 56134757 | 56134915 |
| chr2 | 56282141 | 56282263 |
| chr2 | 56503140 | 56503276 |
| chr2 | 56700936 | 56701060 |
| chr2 | 56777971 | 56778097 |
| chr2 | 57026562 | 57026688 |
| chr2 | 57061613 | 57061757 |
| chr2 | 57279949 | 57280081 |
| chr2 | 57462586 | 57462718 |
| chr2 | 57576974 | 57577122 |
| chr2 | 57652981 | 57653107 |
| chr2 | 57821264 | 57821436 |
| chr2 | 58051685 | 58051857 |
| chr2 | 58173885 | 58174023 |
| chr2 | 58269783 | 58269913 |
| chr2 | 58410932 | 58411058 |
| chr2 | 58577917 | 58578049 |
| chr2 | 58772311 | 58772435 |
| chr2 | 58842856 | 58843018 |
| chr2 | 59090330 | 59090452 |
| chr2 | 59221849 | 59221973 |
| chr2 | 59302902 | 59303030 |
| chr2 | 59444677 | 59444813 |
| chr2 | 59652925 | 59653047 |
| chr2 | 59854463 | 59854591 |
| chr2 | 59991568 | 59991706 |
| chr2 | 60030865 | 60030989 |
| chr2 | 60238567 | 60238707 |
| chr2 | 60353799 | 60353929 |
| chr2 | 60486560 | 60486692 |
| chr2 | 60650690 | 60650828 |
| chr2 | 60815055 | 60815201 |
| chr2 | 60951129 | 60951257 |
| chr2 | 61166946 | 61167076 |
| chr2 | 61338752 | 61338880 |
| chr2 | 61426734 | 61426874 |
| chr2 | 61634648 | 61634776 |
| chr2 | 61747035 | 61747175 |
| chr2 | 61927733 | 61927871 |
| chr2 | 61981558 | 61981680 |
| chr2 | 62238066 | 62238251 |
| chr2 | 62333533 | 62333722 |
| chr2 | 62502311 | 62502441 |
| chr2 | 62666463 | 62666603 |
| chr2 | 62871098 | 62871242 |
| chr2 | 62939307 | 62939431 |
| chr2 | 63132286 | 63132424 |
| chr2 | 63268309 | 63268433 |
| chr2 | 63358184 | 63358308 |
| chr2 | 63490241 | 63490381 |
| chr2 | 63650730 | 63650876 |
| chr2 | 63780517 | 63780643 |
| chr2 | 64008636 | 64008760 |
| chr2 | 64183575 | 64183696 |
| chr2 | 64307230 | 64307368 |
| chr2 | 64518868 | 64519000 |
| chr2 | 64672126 | 64672258 |
| chr2 | 64737154 | 64737282 |
| chr2 | 64881909 | 64882047 |
| chr2 | 64989559 | 64989681 |
| chr2 | 65194216 | 65194354 |
| chr2 | 65355686 | 65355830 |
| chr2 | 65493350 | 65493474 |
| chr2 | 65640157 | 65640299 |
| chr2 | 65861613 | 65861737 |
| chr2 | 65994220 | 65994356 |
| chr2 | 66158817 | 66158945 |
| chr2 | 66277603 | 66277741 |
| chr2 | 66342290 | 66342412 |
| chr2 | 66624408 | 66624532 |
| chr2 | 66726151 | 66726275 |
| chr2 | 66816319 | 66816441 |
| chr2 | 66964738 | 66964880 |
| chr2 | 67086567 | 67086723 |
| chr2 | 67352976 | 67353108 |
| chr2 | 67504076 | 67504210 |
| chr2 | 67622294 | 67622422 |
| chr2 | 67768251 | 67768379 |
| chr2 | 67855242 | 67855384 |
| chr2 | 68051371 | 68051505 |
| chr2 | 68224870 | 68225014 |
| chr2 | 68376314 | 68376458 |
| chr2 | 68475206 | 68475338 |
| chr2 | 68713237 | 68713507 |
| chr2 | 68783932 | 68784054 |
| chr2 | 68967451 | 68967577 |
| chr2 | 69095110 | 69095240 |
| chr2 | 69235470 | 69235610 |
| chr2 | 69387479 | 69387601 |
| chr2 | 69613336 | 69613462 |
| chr2 | 69679566 | 69679690 |
| chr2 | 69813675 | 69813797 |
| chr2 | 69949260 | 69949390 |
| chr2 | 70145675 | 70145805 |
| chr2 | 70234812 | 70234948 |
| chr2 | 70427415 | 70427543 |
| chr2 | 70672077 | 70672201 |
| chr2 | 70759071 | 70759209 |
| chr2 | 70949678 | 70949804 |
| chr2 | 71009513 | 71009647 |
| chr2 | 71176608 | 71176732 |
| chr2 | 71338529 | 71338657 |
| chr2 | 71575191 | 71575315 |
| chr2 | 71701765 | 71701889 |
| chr2 | 71850154 | 71850282 |
| chr2 | 71955720 | 71955860 |
| chr2 | 72136292 | 72136416 |
| chr2 | 72254220 | 72254344 |
| chr2 | 72330270 | 72330410 |
| chr2 | 72482783 | 72482911 |
| chr2 | 72658062 | 72658183 |
| chr2 | 72925115 | 72925236 |
| chr2 | 73016273 | 73016394 |
| chr2 | 73140799 | 73140925 |
| chr2 | 73370947 | 73371079 |
| chr2 | 73464720 | 73464842 |
| chr2 | 73574501 | 73574659 |
| chr2 | 73703915 | 73704043 |
| chr2 | 73885006 | 73885148 |
| chr2 | 74066425 | 74066549 |
| chr2 | 74190198 | 74190334 |
| chr2 | 74369077 | 74369203 |
| chr2 | 74449250 | 74449372 |
| chr2 | 74676089 | 74676227 |
| chr2 | 74831244 | 74831512 |
| chr2 | 74918630 | 74918764 |
| chr2 | 75123997 | 75124123 |
| chr2 | 75307607 | 75307741 |
| chr2 | 75365506 | 75365656 |
| chr2 | 75618509 | 75618633 |
| chr2 | 75722493 | 75722615 |
| chr2 | 75825781 | 75825907 |
| chr2 | 75991742 | 75991864 |
| chr2 | 76163138 | 76163272 |
| chr2 | 76339674 | 76339832 |
| chr2 | 76473226 | 76473376 |
| chr2 | 76540275 | 76540417 |
| chr2 | 76724844 | 76724984 |
| chr2 | 76870663 | 76870797 |
| chr2 | 77020162 | 77020296 |
| chr2 | 77236287 | 77236429 |
| chr2 | 77397037 | 77397163 |
| chr2 | 77440430 | 77440580 |
| chr2 | 77591516 | 77591662 |
| chr2 | 77861709 | 77861837 |
| chr2 | 77902178 | 77902348 |
| chr2 | 77919254 | 77919376 |
| chr2 | 78174687 | 78174853 |
| chr2 | 78240838 | 78240970 |
| chr2 | 78353490 | 78353642 |
| chr2 | 78605260 | 78605396 |
| chr2 | 78734823 | 78734945 |
| chr2 | 78924340 | 78924466 |
| chr2 | 78960813 | 78960967 |
| chr2 | 79095500 | 79095640 |
| chr2 | 79287360 | 79287498 |
| chr2 | 79418435 | 79418557 |
| chr2 | 79608928 | 79609054 |
| chr2 | 79689490 | 79689614 |
| chr2 | 79971883 | 79972013 |
| chr2 | 80066438 | 80066574 |
| chr2 | 80168032 | 80168172 |
| chr2 | 80301507 | 80301657 |
| chr2 | 80445665 | 80445797 |
| chr2 | 80716601 | 80716743 |
| chr2 | 80737938 | 80738062 |
| chr2 | 81004570 | 81004698 |
| chr2 | 81089926 | 81090056 |
| chr2 | 81287201 | 81287345 |
| chr2 | 81476185 | 81476319 |
| chr2 | 81572155 | 81572289 |
| chr2 | 81673967 | 81674095 |
| chr2 | 81891888 | 81892012 |
| chr2 | 82003445 | 82003573 |
| chr2 | 82171771 | 82171897 |
| chr2 | 82235596 | 82235732 |
| chr2 | 82514715 | 82514979 |
| chr2 | 82609753 | 82609882 |
| chr2 | 82737816 | 82737937 |
| chr2 | 82956360 | 82956600 |
| chr2 | 83120655 | 83120797 |
| chr2 | 83233449 | 83233577 |
| chr2 | 83406279 | 83406411 |
| chr2 | 83541212 | 83541336 |
| chr2 | 83714560 | 83714684 |
| chr2 | 83860113 | 83860249 |
| chr2 | 83915419 | 83915551 |
| chr2 | 84061887 | 84062037 |
| chr2 | 84263601 | 84263725 |
| chr2 | 84380110 | 84380234 |
| chr2 | 84565471 | 84565613 |
| chr2 | 84766793 | 84766914 |
| chr2 | 84924298 | 84924426 |
| chr2 | 85065545 | 85065695 |
| chr2 | 85228555 | 85228808 |
| chr2 | 85321704 | 85321834 |
| chr2 | 85443584 | 85443732 |
| chr2 | 85655924 | 85656056 |
| chr2 | 85738286 | 85738418 |
| chr2 | 85831854 | 85832104 |
| chr2 | 86028319 | 86028465 |
| chr2 | 86149452 | 86149592 |
| chr2 | 86414024 | 86414166 |
| chr2 | 86485060 | 86485182 |
| chr2 | 86630130 | 86630254 |
| chr2 | 86859260 | 86859386 |
| chr2 | 86923088 | 86923226 |
| chr2 | 87059104 | 87059228 |
| chr2 | 87322936 | 87323106 |
| chr2 | 87411466 | 87411604 |
| chr2 | 87542828 | 87542968 |
| chr2 | 87597790 | 87597912 |
| chr2 | 87605324 | 87605480 |
| chr2 | 87765778 | 87765906 |
| chr2 | 87808948 | 87809082 |
| chr2 | 87820925 | 87821057 |
| chr2 | 87940030 | 87940170 |
| chr2 | 88161182 | 88161306 |
| chr2 | 88211115 | 88211241 |
| chr2 | 88306242 | 88306378 |
| chr2 | 88528681 | 88528805 |
| chr2 | 88647687 | 88647829 |
| chr2 | 88794370 | 88794520 |
| chr2 | 88937091 | 88937215 |
| chr2 | 89040395 | 89040537 |
| chr2 | 89172321 | 89172445 |
| chr2 | 89301699 | 89301819 |
| chr2 | 89435661 | 89435791 |
| chr2 | 89450893 | 89451035 |
| chr2 | 89525333 | 89525457 |
| chr2 | 89883641 | 89883762 |
| chr2 | 89995282 | 89995406 |
| chr2 | 90069186 | 90069328 |
| chr2 | 90084576 | 90084706 |
| chr2 | 90237379 | 90237507 |
| chr2 | 90301559 | 90301681 |
| chr2 | 90309124 | 90309244 |
| chr2 | 90427718 | 90427842 |
| chr2 | 90445027 | 90445175 |
| chr2 | 90503652 | 90503772 |
| chr2 | 90511215 | 90511337 |
| chr2 | 91649493 | 91649617 |
| chr2 | 91666867 | 91667015 |
| chr2 | 91725333 | 91725489 |
| chr2 | 91885458 | 91885588 |
| chr2 | 91984386 | 91984524 |
| chr2 | 91994464 | 91994612 |
| chr2 | 92007191 | 92007315 |
| chr2 | 92221506 | 92221630 |
| chr2 | 92234211 | 92234359 |
| chr2 | 92244305 | 92244443 |
| chr2 | 92251693 | 92251812 |
| chr2 | 92267093 | 92267213 |
| chr2 | 95326693 | 95326815 |
| chr2 | 95335116 | 95335266 |
| chr2 | 95582686 | 95582948 |
| chr2 | 95614389 | 95614517 |
| chr2 | 95710782 | 95710906 |
| chr2 | 95784266 | 95784400 |
| chr2 | 95926070 | 95926210 |
| chr2 | 96063461 | 96063585 |
| chr2 | 96237726 | 96237847 |
| chr2 | 96404363 | 96404489 |
| chr2 | 96439431 | 96439551 |
| chr2 | 96532751 | 96532885 |
| chr2 | 96596196 | 96596320 |
| chr2 | 96716526 | 96716646 |
| chr2 | 96739881 | 96740003 |
| chr2 | 96925191 | 96925323 |
| chr2 | 97006629 | 97006771 |
| chr2 | 97207237 | 97207379 |
| chr2 | 97277405 | 97277529 |
| chr2 | 97380153 | 97380277 |
| chr2 | 97671092 | 97671271 |
| chr2 | 97713873 | 97713995 |
| chr2 | 97839871 | 97839995 |
| chr2 | 97899911 | 97900045 |
| chr2 | 97999495 | 97999617 |
| chr2 | 98024731 | 98024999 |
| chr2 | 98039274 | 98039396 |
| chr2 | 98139351 | 98139485 |
| chr2 | 98178504 | 98178628 |
| chr2 | 98223299 | 98223451 |
| chr2 | 98407306 | 98407430 |
| chr2 | 98545803 | 98545929 |
| chr2 | 98706978 | 98707118 |
| chr2 | 98813459 | 98813597 |
| chr2 | 98924831 | 98924955 |
| chr2 | 99108193 | 99108319 |
| chr2 | 99304758 | 99304882 |
| chr2 | 99383982 | 99384108 |
| chr2 | 99589745 | 99589883 |
| chr2 | 99637182 | 99637312 |
| chr2 | 99886229 | 99886371 |
| chr2 | 100065285 | 100065435 |
| chr2 | 100195114 | 100195240 |
| chr2 | 100307019 | 100307143 |
| chr2 | 100417001 | 100417129 |
| chr2 | 100636675 | 100636813 |
| chr2 | 100803724 | 100803848 |
| chr2 | 100933986 | 100934114 |
| chr2 | 101057967 | 101058093 |
| chr2 | 101234364 | 101234510 |
| chr2 | 101286187 | 101286319 |
| chr2 | 101569340 | 101569472 |
| chr2 | 101634597 | 101634721 |
| chr2 | 101778245 | 101778367 |
| chr2 | 101917371 | 101917495 |
| chr2 | 102037765 | 102037907 |
| chr2 | 102294836 | 102294972 |
| chr2 | 102378708 | 102378832 |
| chr2 | 102589822 | 102589964 |
| chr2 | 102696976 | 102697100 |
| chr2 | 102801806 | 102801930 |
| chr2 | 102960205 | 102960335 |
| chr2 | 103177326 | 103177494 |
| chr2 | 103289494 | 103289620 |
| chr2 | 103445870 | 103446014 |
| chr2 | 103644384 | 103644507 |
| chr2 | 103791767 | 103791895 |
| chr2 | 103943315 | 103943455 |
| chr2 | 104044195 | 104044316 |
| chr2 | 104259893 | 104260019 |
| chr2 | 104392444 | 104392570 |
| chr2 | 104502112 | 104502236 |
| chr2 | 104630113 | 104630237 |
| chr2 | 104807680 | 104807804 |
| chr2 | 104982495 | 104982639 |
| chr2 | 105103270 | 105103396 |
| chr2 | 105284825 | 105284961 |
| chr2 | 105358583 | 105358725 |
| chr2 | 105616252 | 105616416 |
| chr2 | 105698897 | 105699027 |
| chr2 | 105819471 | 105819605 |
| chr2 | 106021412 | 106021536 |
| chr2 | 106137536 | 106137670 |
| chr2 | 106365936 | 106366072 |
| chr2 | 106505442 | 106505566 |
| chr2 | 106636652 | 106636810 |
| chr2 | 106815939 | 106816075 |
| chr2 | 106909174 | 106909298 |
| chr2 | 106983323 | 106983518 |
| chr2 | 107216900 | 107217024 |
| chr2 | 107301105 | 107301227 |
| chr2 | 107523718 | 107523844 |
| chr2 | 107650705 | 107650829 |
| chr2 | 107813415 | 107813545 |
| chr2 | 107945972 | 107946100 |
| chr2 | 108101329 | 108101479 |
| chr2 | 108228526 | 108228650 |
| chr2 | 108422736 | 108422864 |
| chr2 | 108542529 | 108542651 |
| chr2 | 108644945 | 108645087 |
| chr2 | 108818826 | 108818964 |
| chr2 | 108970843 | 108971110 |
| chr2 | 109103419 | 109103559 |
| chr2 | 109256783 | 109256904 |
| chr2 | 109408763 | 109408884 |
| chr2 | 109599204 | 109599326 |
| chr2 | 109794974 | 109795120 |
| chr2 | 109866659 | 109866791 |
| chr2 | 110029244 | 110029380 |
| chr2 | 110254757 | 110254881 |
| chr2 | 110414631 | 110414771 |
| chr2 | 110492978 | 110493104 |
| chr2 | 110695590 | 110695712 |
| chr2 | 110862476 | 110862600 |
| chr2 | 110970221 | 110970468 |
| chr2 | 110993535 | 110993657 |
| chr2 | 111190949 | 111191071 |
| chr2 | 111396043 | 111396167 |
| chr2 | 111497649 | 111497901 |
| chr2 | 111639553 | 111639677 |
| chr2 | 111836464 | 111836598 |
| chr2 | 112007881 | 112008021 |
| chr2 | 112067765 | 112067905 |
| chr2 | 112186865 | 112186997 |
| chr2 | 112198848 | 112198982 |
| chr2 | 112241393 | 112241521 |
| chr2 | 112303350 | 112303490 |
| chr2 | 112376927 | 112377072 |
| chr2 | 112474817 | 112474938 |
| chr2 | 112538562 | 112538682 |
| chr2 | 112653935 | 112654059 |
| chr2 | 112685570 | 112685708 |
| chr2 | 112899419 | 112899557 |
| chr2 | 113008959 | 113009085 |
| chr2 | 113207629 | 113207751 |
| chr2 | 113424554 | 113424678 |
| chr2 | 113569878 | 113570008 |
| chr2 | 113627198 | 113627320 |
| chr2 | 113740199 | 113740337 |
| chr2 | 113981081 | 113981225 |
| chr2 | 114137759 | 114137895 |
| chr2 | 114209107 | 114209229 |
| chr2 | 114239022 | 114239166 |
| chr2 | 114293395 | 114293521 |
| chr2 | 114300120 | 114300244 |
| chr2 | 114314018 | 114314146 |
| chr2 | 114359289 | 114359407 |
| chr2 | 114380499 | 114380629 |
| chr2 | 114429878 | 114429999 |
| chr2 | 114554978 | 114555114 |
| chr2 | 114667412 | 114667578 |
| chr2 | 114927972 | 114928120 |
| chr2 | 115013445 | 115013601 |
| chr2 | 115208420 | 115208568 |
| chr2 | 115326221 | 115326361 |
| chr2 | 115430824 | 115430964 |
| chr2 | 115652876 | 115653008 |
| chr2 | 115758397 | 115758537 |
| chr2 | 115969846 | 115969984 |
| chr2 | 116028314 | 116028448 |
| chr2 | 116202292 | 116202422 |
| chr2 | 116377103 | 116377233 |
| chr2 | 116564884 | 116565014 |
| chr2 | 116605099 | 116605225 |
| chr2 | 116867512 | 116867656 |
| chr2 | 116985790 | 116985928 |
| chr2 | 117124609 | 117124747 |
| chr2 | 117187118 | 117187262 |
| chr2 | 117441465 | 117441597 |
| chr2 | 117507347 | 117507469 |
| chr2 | 117772582 | 117772734 |
| chr2 | 117808012 | 117808140 |
| chr2 | 118073764 | 118073888 |
| chr2 | 118104534 | 118104692 |
| chr2 | 118363237 | 118363363 |
| chr2 | 118461051 | 118461175 |
| chr2 | 118584739 | 118584861 |
| chr2 | 118718205 | 118718331 |
| chr2 | 118877944 | 118878090 |
| chr2 | 119015994 | 119016126 |
| chr2 | 119218791 | 119218941 |
| chr2 | 119337507 | 119337633 |
| chr2 | 119489339 | 119489465 |
| chr2 | 119668336 | 119668460 |
| chr2 | 119741011 | 119741147 |
| chr2 | 119900057 | 119900181 |
| chr2 | 120103057 | 120103201 |
| chr2 | 120213407 | 120213543 |
| chr2 | 120457962 | 120458108 |
| chr2 | 120578116 | 120578240 |
| chr2 | 120669803 | 120669943 |
| chr2 | 120858684 | 120858832 |
| chr2 | 120958207 | 120958337 |
| chr2 | 121098257 | 121098405 |
| chr2 | 121290582 | 121290720 |
| chr2 | 121456815 | 121456947 |
| chr2 | 121597988 | 121598258 |
| chr2 | 121682132 | 121682258 |
| chr2 | 121918725 | 121918869 |
| chr2 | 122048858 | 122048982 |
| chr2 | 122187277 | 122187405 |
| chr2 | 122354565 | 122354689 |
| chr2 | 122528925 | 122529208 |
| chr2 | 122697171 | 122697309 |
| chr2 | 122820324 | 122820448 |
| chr2 | 123013059 | 123013181 |
| chr2 | 123094927 | 123095059 |
| chr2 | 123284950 | 123285076 |
| chr2 | 123447208 | 123447334 |
| chr2 | 123619647 | 123619781 |
| chr2 | 123745485 | 123745609 |
| chr2 | 123835216 | 123835348 |
| chr2 | 124055132 | 124055258 |
| chr2 | 124196032 | 124196156 |
| chr2 | 124312275 | 124312417 |
| chr2 | 124430367 | 124430519 |
| chr2 | 124540482 | 124540626 |
| chr2 | 124729658 | 124729782 |
| chr2 | 124869625 | 124869757 |
| chr2 | 125004017 | 125004159 |
| chr2 | 125197229 | 125197367 |
| chr2 | 125332489 | 125332633 |
| chr2 | 125446138 | 125446278 |
| chr2 | 125722354 | 125722506 |
| chr2 | 125775532 | 125775674 |
| chr2 | 125937258 | 125937382 |
| chr2 | 126080122 | 126080250 |
| chr2 | 126184949 | 126185075 |
| chr2 | 126401430 | 126401564 |
| chr2 | 126549735 | 126549861 |
| chr2 | 126692292 | 126692442 |
| chr2 | 126897557 | 126897683 |
| chr2 | 127045289 | 127045429 |
| chr2 | 127194754 | 127194908 |
| chr2 | 127345725 | 127345849 |
| chr2 | 127503457 | 127503581 |
| chr2 | 127537496 | 127537636 |
| chr2 | 127705521 | 127705659 |
| chr2 | 127974853 | 127974987 |
| chr2 | 128060073 | 128060217 |
| chr2 | 128169855 | 128169979 |
| chr2 | 128356923 | 128357059 |
| chr2 | 128496249 | 128496373 |
| chr2 | 128633166 | 128633310 |
| chr2 | 128741882 | 128742024 |
| chr2 | 128885721 | 128885849 |
| chr2 | 129032987 | 129033123 |
| chr2 | 129291120 | 129291248 |
| chr2 | 129436887 | 129437027 |
| chr2 | 129526803 | 129526927 |
| chr2 | 129635438 | 129635560 |
| chr2 | 129785912 | 129786036 |
| chr2 | 129985506 | 129985630 |
| chr2 | 130196300 | 130196450 |
| chr2 | 130323670 | 130323804 |
| chr2 | 130387721 | 130387861 |
| chr2 | 130535152 | 130535288 |
| chr2 | 130781124 | 130781278 |
| chr2 | 130868730 | 130868874 |
| chr2 | 131123174 | 131123300 |
| chr2 | 131143049 | 131143170 |
| chr2 | 131284929 | 131285107 |
| chr2 | 131350810 | 131350988 |
| chr2 | 131492713 | 131492835 |
| chr2 | 131642112 | 131642234 |
| chr2 | 131833722 | 131833844 |
| chr2 | 131889089 | 131889211 |
| chr2 | 131985195 | 131985339 |
| chr2 | 132162952 | 132163082 |
| chr2 | 132324395 | 132324527 |
| chr2 | 132358376 | 132358519 |
| chr2 | 132365501 | 132365625 |
| chr2 | 132525003 | 132525123 |
| chr2 | 132552371 | 132552507 |
| chr2 | 132642351 | 132642497 |
| chr2 | 132840081 | 132840201 |
| chr2 | 132846341 | 132846461 |
| chr2 | 132858969 | 132859129 |
| chr2 | 132932445 | 132932573 |
| chr2 | 133052068 | 133052196 |
| chr2 | 133209046 | 133209174 |
| chr2 | 133278302 | 133278436 |
| chr2 | 133512815 | 133512939 |
| chr2 | 133619540 | 133619668 |
| chr2 | 133743709 | 133743841 |
| chr2 | 133866938 | 133867070 |
| chr2 | 134000022 | 134000148 |
| chr2 | 134167604 | 134167734 |
| chr2 | 134413929 | 134414053 |
| chr2 | 134516654 | 134516778 |
| chr2 | 134690243 | 134690375 |
| chr2 | 134738357 | 134738497 |
| chr2 | 134954673 | 134954815 |
| chr2 | 135031807 | 135031949 |
| chr2 | 135189549 | 135189673 |
| chr2 | 135453980 | 135454102 |
| chr2 | 135616545 | 135616669 |
| chr2 | 135753946 | 135754072 |
| chr2 | 135818871 | 135818995 |
| chr2 | 136002418 | 136002554 |
| chr2 | 136080754 | 136080900 |
| chr2 | 136370614 | 136370868 |
| chr2 | 136501784 | 136501924 |
| chr2 | 136536005 | 136536131 |
| chr2 | 136714114 | 136714264 |
| chr2 | 136932265 | 136932401 |
| chr2 | 137128083 | 137128207 |
| chr2 | 137173279 | 137173415 |
| chr2 | 137414730 | 137414870 |
| chr2 | 137561704 | 137561838 |
| chr2 | 137591958 | 137592110 |
| chr2 | 137842050 | 137842178 |
| chr2 | 137975070 | 137975200 |
| chr2 | 138037582 | 138037716 |
| chr2 | 138280386 | 138280534 |
| chr2 | 138459492 | 138459634 |
| chr2 | 138485762 | 138485908 |
| chr2 | 138631621 | 138631749 |
| chr2 | 138823360 | 138823482 |
| chr2 | 139076665 | 139076801 |
| chr2 | 139102614 | 139102738 |
| chr2 | 139341278 | 139341557 |
| chr2 | 139489298 | 139489450 |
| chr2 | 139646180 | 139646308 |
| chr2 | 139731072 | 139731196 |
| chr2 | 139963802 | 139963940 |
| chr2 | 140031140 | 140031288 |
| chr2 | 140152140 | 140152264 |
| chr2 | 140332882 | 140333006 |
| chr2 | 140494336 | 140494458 |
| chr2 | 140676395 | 140676567 |
| chr2 | 140759715 | 140759839 |
| chr2 | 140945986 | 140946108 |
| chr2 | 141152991 | 141153112 |
| chr2 | 141272493 | 141272615 |
| chr2 | 141439732 | 141439860 |
| chr2 | 141627812 | 141627952 |
| chr2 | 141742653 | 141742797 |
| chr2 | 141799841 | 141799965 |
| chr2 | 142015550 | 142015674 |
| chr2 | 142158108 | 142158248 |
| chr2 | 142274866 | 142274988 |
| chr2 | 142488477 | 142488615 |
| chr2 | 142536751 | 142536875 |
| chr2 | 142713241 | 142713367 |
| chr2 | 142885828 | 142885954 |
| chr2 | 143029587 | 143029739 |
| chr2 | 143135974 | 143136114 |
| chr2 | 143322920 | 143323052 |
| chr2 | 143475744 | 143475880 |
| chr2 | 143638548 | 143638676 |
| chr2 | 143826756 | 143826886 |
| chr2 | 143925435 | 143925571 |
| chr2 | 144158091 | 144158221 |
| chr2 | 144248819 | 144248941 |
| chr2 | 144386817 | 144386951 |
| chr2 | 144591374 | 144591520 |
| chr2 | 144654241 | 144654363 |
| chr2 | 144782863 | 144783133 |
| chr2 | 145059531 | 145059791 |
| chr2 | 145173687 | 145173813 |
| chr2 | 145236060 | 145236204 |
| chr2 | 145524182 | 145524318 |
| chr2 | 145568105 | 145568231 |
| chr2 | 145711654 | 145711808 |
| chr2 | 145884806 | 145884936 |
| chr2 | 146023571 | 146023695 |
| chr2 | 146202427 | 146202552 |
| chr2 | 146356724 | 146356850 |
| chr2 | 146455779 | 146455917 |
| chr2 | 146582192 | 146582318 |
| chr2 | 146779159 | 146779309 |
| chr2 | 146939103 | 146939243 |
| chr2 | 147107606 | 147107728 |
| chr2 | 147292604 | 147292730 |
| chr2 | 147383681 | 147383875 |
| chr2 | 147497913 | 147498039 |
| chr2 | 147674115 | 147674237 |
| chr2 | 147849156 | 147849280 |
| chr2 | 147960325 | 147960461 |
| chr2 | 148102100 | 148102275 |
| chr2 | 148282580 | 148282716 |
| chr2 | 148390558 | 148390698 |
| chr2 | 148593780 | 148593910 |
| chr2 | 148785238 | 148785374 |
| chr2 | 148964407 | 148964531 |
| chr2 | 149055772 | 149055898 |
| chr2 | 149251636 | 149251774 |
| chr2 | 149325571 | 149325711 |
| chr2 | 149553101 | 149553225 |
| chr2 | 149675091 | 149675211 |
| chr2 | 149792372 | 149792512 |
| chr2 | 149970389 | 149970533 |
| chr2 | 150058939 | 150059069 |
| chr2 | 150328860 | 150328984 |
| chr2 | 150365331 | 150365457 |
| chr2 | 150564016 | 150564164 |
| chr2 | 150663736 | 150663860 |
| chr2 | 150796313 | 150796443 |
| chr2 | 150932507 | 150932641 |
| chr2 | 151219047 | 151219177 |
| chr2 | 151259903 | 151260033 |
| chr2 | 151401705 | 151401845 |
| chr2 | 151608697 | 151608821 |
| chr2 | 151765107 | 151765241 |
| chr2 | 151838815 | 151838943 |
| chr2 | 152126391 | 152126555 |
| chr2 | 152180436 | 152180558 |
| chr2 | 152301252 | 152301376 |
| chr2 | 152567818 | 152567942 |
| chr2 | 152707540 | 152707668 |
| chr2 | 152754148 | 152754290 |
| chr2 | 153003376 | 153003500 |
| chr2 | 153164702 | 153164848 |
| chr2 | 153257565 | 153257689 |
| chr2 | 153393885 | 153394035 |
| chr2 | 153553895 | 153554061 |
| chr2 | 153672740 | 153672862 |
| chr2 | 153882000 | 153882126 |
| chr2 | 154060731 | 154060859 |
| chr2 | 154161878 | 154162008 |
| chr2 | 154302723 | 154302845 |
| chr2 | 154423377 | 154423511 |
| chr2 | 154540135 | 154540259 |
| chr2 | 154759304 | 154759428 |
| chr2 | 154953087 | 154953229 |
| chr2 | 155109722 | 155109852 |
| chr2 | 155177783 | 155177907 |
| chr2 | 155366433 | 155366555 |
| chr2 | 155450106 | 155450228 |
| chr2 | 155656890 | 155657036 |
| chr2 | 155873108 | 155873250 |
| chr2 | 155954102 | 155954234 |
| chr2 | 156159370 | 156159494 |
| chr2 | 156218800 | 156218940 |
| chr2 | 156376763 | 156376905 |
| chr2 | 156550512 | 156550654 |
| chr2 | 156636441 | 156636583 |
| chr2 | 156911292 | 156911430 |
| chr2 | 157043764 | 157043896 |
| chr2 | 157147657 | 157147813 |
| chr2 | 157324098 | 157324222 |
| chr2 | 157457510 | 157457640 |
| chr2 | 157603862 | 157604016 |
| chr2 | 157740648 | 157740772 |
| chr2 | 157902312 | 157902440 |
| chr2 | 158009285 | 158009415 |
| chr2 | 158222900 | 158223021 |
| chr2 | 158352698 | 158352828 |
| chr2 | 158542694 | 158542836 |
| chr2 | 158726495 | 158726637 |
| chr2 | 158771992 | 158772144 |
| chr2 | 158888781 | 158888905 |
| chr2 | 159166241 | 159166379 |
| chr2 | 159230498 | 159230640 |
| chr2 | 159361662 | 159361786 |
| chr2 | 159512448 | 159512590 |
| chr2 | 159772365 | 159772511 |
| chr2 | 159914716 | 159914870 |
| chr2 | 159983010 | 159983138 |
| chr2 | 160117945 | 160118093 |
| chr2 | 160275364 | 160275488 |
| chr2 | 160467043 | 160467165 |
| chr2 | 160548129 | 160548273 |
| chr2 | 160787168 | 160787310 |
| chr2 | 160895777 | 160895907 |
| chr2 | 161044755 | 161044893 |
| chr2 | 161147655 | 161147791 |
| chr2 | 161294258 | 161294382 |
| chr2 | 161437138 | 161437282 |
| chr2 | 161672533 | 161672675 |
| chr2 | 161847151 | 161847275 |
| chr2 | 161975317 | 161975471 |
| chr2 | 162131105 | 162131239 |
| chr2 | 162294451 | 162294575 |
| chr2 | 162408333 | 162408458 |
| chr2 | 162570957 | 162571079 |
| chr2 | 162699557 | 162699764 |
| chr2 | 162789100 | 162789238 |
| chr2 | 162944416 | 162944548 |
| chr2 | 163186965 | 163187166 |
| chr2 | 163373502 | 163373624 |
| chr2 | 163430597 | 163430721 |
| chr2 | 163657005 | 163657131 |
| chr2 | 163698122 | 163698252 |
| chr2 | 163931255 | 163931381 |
| chr2 | 163999548 | 163999694 |
| chr2 | 164149614 | 164149738 |
| chr2 | 164344285 | 164344415 |
| chr2 | 164567615 | 164567737 |
| chr2 | 164621448 | 164621580 |
| chr2 | 164734131 | 164734257 |
| chr2 | 164918798 | 164918934 |
| chr2 | 165059740 | 165059872 |
| chr2 | 165213449 | 165213579 |
| chr2 | 165411758 | 165411880 |
| chr2 | 165609304 | 165609436 |
| chr2 | 165761919 | 165762073 |
| chr2 | 165819225 | 165819365 |
| chr2 | 166059833 | 166059959 |
| chr2 | 166213241 | 166213363 |
| chr2 | 166336606 | 166336746 |
| chr2 | 166414024 | 166414180 |
| chr2 | 166559984 | 166560118 |
| chr2 | 166738563 | 166738701 |
| chr2 | 166847412 | 166847534 |
| chr2 | 167011378 | 167011504 |
| chr2 | 167247884 | 167248008 |
| chr2 | 167424647 | 167424789 |
| chr2 | 167515134 | 167515274 |
| chr2 | 167666655 | 167666779 |
| chr2 | 167785895 | 167786043 |
| chr2 | 167944951 | 167945075 |
| chr2 | 168066455 | 168066579 |
| chr2 | 168314090 | 168314357 |
| chr2 | 168477404 | 168477556 |
| chr2 | 168574485 | 168574611 |
| chr2 | 168772432 | 168772562 |
| chr2 | 168858417 | 168858541 |
| chr2 | 169048003 | 169048147 |
| chr2 | 169198017 | 169198153 |
| chr2 | 169279220 | 169279352 |
| chr2 | 169501072 | 169501202 |
| chr2 | 169557375 | 169557499 |
| chr2 | 169794643 | 169794767 |
| chr2 | 169884566 | 169884706 |
| chr2 | 170001854 | 170001980 |
| chr2 | 170201167 | 170201293 |
| chr2 | 170393186 | 170393312 |
| chr2 | 170484692 | 170484840 |
| chr2 | 170588640 | 170588776 |
| chr2 | 170739885 | 170740027 |
| chr2 | 170962866 | 170962990 |
| chr2 | 171060639 | 171060791 |
| chr2 | 171287648 | 171287784 |
| chr2 | 171373496 | 171373638 |
| chr2 | 171556975 | 171557097 |
| chr2 | 171655671 | 171655811 |
| chr2 | 171840169 | 171840291 |
| chr2 | 171978111 | 171978261 |
| chr2 | 172204271 | 172204393 |
| chr2 | 172334942 | 172335074 |
| chr2 | 172397625 | 172397747 |
| chr2 | 172645081 | 172645587 |
| chr2 | 172745723 | 172745851 |
| chr2 | 172969023 | 172969146 |
| chr2 | 173107075 | 173107209 |
| chr2 | 173219782 | 173219922 |
| chr2 | 173375863 | 173375997 |
| chr2 | 173480538 | 173480664 |
| chr2 | 173585149 | 173585287 |
| chr2 | 173877446 | 173877582 |
| chr2 | 173912805 | 173912947 |
| chr2 | 174067376 | 174067516 |
| chr2 | 174290722 | 174290856 |
| chr2 | 174462737 | 174462867 |
| chr2 | 174516001 | 174516129 |
| chr2 | 174761547 | 174761681 |
| chr2 | 174868705 | 174868829 |
| chr2 | 174971419 | 174971543 |
| chr2 | 175093143 | 175093267 |
| chr2 | 175295681 | 175295805 |
| chr2 | 175495403 | 175495531 |
| chr2 | 175665177 | 175665303 |
| chr2 | 175744536 | 175744672 |
| chr2 | 175895680 | 175895830 |
| chr2 | 176005973 | 176006111 |
| chr2 | 176187974 | 176188100 |
| chr2 | 176335257 | 176335391 |
| chr2 | 176496899 | 176497033 |
| chr2 | 176684559 | 176684683 |
| chr2 | 176838061 | 176838185 |
| chr2 | 176984492 | 176984787 |
| chr2 | 177030835 | 177030973 |
| chr2 | 177260017 | 177260149 |
| chr2 | 177348349 | 177348477 |
| chr2 | 177573058 | 177573182 |
| chr2 | 177737070 | 177737257 |
| chr2 | 177924282 | 177924416 |
| chr2 | 178066298 | 178066422 |
| chr2 | 178167171 | 178167297 |
| chr2 | 178309766 | 178309910 |
| chr2 | 178406397 | 178406519 |
| chr2 | 178640178 | 178640318 |
| chr2 | 178689149 | 178689287 |
| chr2 | 178852078 | 178852230 |
| chr2 | 179084325 | 179084479 |
| chr2 | 179212906 | 179213050 |
| chr2 | 179361115 | 179361255 |
| chr2 | 179461133 | 179461283 |
| chr2 | 179680167 | 179680303 |
| chr2 | 179840866 | 179840990 |
| chr2 | 179971004 | 179971154 |
| chr2 | 180122957 | 180123111 |
| chr2 | 180197776 | 180197910 |
| chr2 | 180332621 | 180332767 |
| chr2 | 180597842 | 180597984 |
| chr2 | 180653498 | 180653622 |
| chr2 | 180850830 | 180850952 |
| chr2 | 180962564 | 180962712 |
| chr2 | 181120231 | 181120389 |
| chr2 | 181257089 | 181257223 |
| chr2 | 181438808 | 181438948 |
| chr2 | 181554486 | 181554622 |
| chr2 | 181725510 | 181725646 |
| chr2 | 181913167 | 181913315 |
| chr2 | 182029282 | 182029406 |
| chr2 | 182261825 | 182261949 |
| chr2 | 182368862 | 182368992 |
| chr2 | 182456741 | 182456887 |
| chr2 | 182650869 | 182650995 |
| chr2 | 182741391 | 182741683 |
| chr2 | 183023396 | 183023536 |
| chr2 | 183165944 | 183166072 |
| chr2 | 183206272 | 183206428 |
| chr2 | 183472614 | 183472744 |
| chr2 | 183537337 | 183537485 |
| chr2 | 183667608 | 183667730 |
| chr2 | 183896233 | 183896486 |
| chr2 | 183985525 | 183985679 |
| chr2 | 184080708 | 184080860 |
| chr2 | 184342130 | 184342280 |
| chr2 | 184391900 | 184392070 |
| chr2 | 184608325 | 184608449 |
| chr2 | 184823432 | 184823584 |
| chr2 | 184975721 | 184975851 |
| chr2 | 185006968 | 185007124 |
| chr2 | 185166328 | 185166468 |
| chr2 | 185376243 | 185376367 |
| chr2 | 185546086 | 185546209 |
| chr2 | 185724395 | 185724523 |
| chr2 | 185842241 | 185842387 |
| chr2 | 185890127 | 185890248 |
| chr2 | 186062281 | 186062409 |
| chr2 | 186260393 | 186260515 |
| chr2 | 186374378 | 186374520 |
| chr2 | 186628161 | 186628285 |
| chr2 | 186735852 | 186735974 |
| chr2 | 186848662 | 186848786 |
| chr2 | 186970133 | 186970271 |
| chr2 | 187212552 | 187212704 |
| chr2 | 187364017 | 187364149 |
| chr2 | 187401368 | 187401508 |
| chr2 | 187633313 | 187633437 |
| chr2 | 187756027 | 187756161 |
| chr2 | 187880030 | 187880154 |
| chr2 | 188022714 | 188022846 |
| chr2 | 188185993 | 188186117 |
| chr2 | 188353581 | 188353723 |
| chr2 | 188573662 | 188573790 |
| chr2 | 188663293 | 188663419 |
| chr2 | 188764801 | 188764925 |
| chr2 | 188974284 | 188974436 |
| chr2 | 189136987 | 189137111 |
| chr2 | 189253307 | 189253431 |
| chr2 | 189425724 | 189425846 |
| chr2 | 189531538 | 189531661 |
| chr2 | 189743016 | 189743142 |
| chr2 | 189816093 | 189816221 |
| chr2 | 190047089 | 190047231 |
| chr2 | 190101182 | 190101310 |
| chr2 | 190254722 | 190254846 |
| chr2 | 190479742 | 190479894 |
| chr2 | 190649492 | 190649636 |
| chr2 | 190816234 | 190816360 |
| chr2 | 190959449 | 190959571 |
| chr2 | 191008114 | 191008242 |
| chr2 | 191270834 | 191270976 |
| chr2 | 191327792 | 191327922 |
| chr2 | 191441515 | 191441657 |
| chr2 | 191706664 | 191706794 |
| chr2 | 191808651 | 191808795 |
| chr2 | 191935742 | 191935890 |
| chr2 | 192085051 | 192085209 |
| chr2 | 192274995 | 192275273 |
| chr2 | 192376954 | 192377080 |
| chr2 | 192614029 | 192614161 |
| chr2 | 192657182 | 192657352 |
| chr2 | 192880484 | 192880616 |
| chr2 | 193028504 | 193028632 |
| chr2 | 193167410 | 193167554 |
| chr2 | 193346192 | 193346314 |
| chr2 | 193420616 | 193420740 |
| chr2 | 193586980 | 193587122 |
| chr2 | 193764838 | 193764986 |
| chr2 | 193974040 | 193974190 |
| chr2 | 194019070 | 194019190 |
| chr2 | 194173722 | 194173868 |
| chr2 | 194327534 | 194327660 |
| chr2 | 194473730 | 194473852 |
| chr2 | 194722601 | 194722722 |
| chr2 | 194805191 | 194805319 |
| chr2 | 194971675 | 194971916 |
| chr2 | 195060389 | 195060662 |
| chr2 | 195266158 | 195266282 |
| chr2 | 195469178 | 195469314 |
| chr2 | 195622102 | 195622226 |
| chr2 | 195764814 | 195764936 |
| chr2 | 195914406 | 195914552 |
| chr2 | 196018004 | 196018132 |
| chr2 | 196102507 | 196102631 |
| chr2 | 196335045 | 196335193 |
| chr2 | 196400026 | 196400182 |
| chr2 | 196571762 | 196571888 |
| chr2 | 196779085 | 196779219 |
| chr2 | 196917269 | 196917417 |
| chr2 | 197104498 | 197104632 |
| chr2 | 197215359 | 197215499 |
| chr2 | 197284228 | 197284358 |
| chr2 | 197504782 | 197504914 |
| chr2 | 197579691 | 197579815 |
| chr2 | 197808009 | 197808266 |
| chr2 | 197957280 | 197957402 |
| chr2 | 198057259 | 198057401 |
| chr2 | 198233622 | 198233746 |
| chr2 | 198472828 | 198472974 |
| chr2 | 198545424 | 198545550 |
| chr2 | 198665468 | 198665592 |
| chr2 | 198909658 | 198909790 |
| chr2 | 199032742 | 199032874 |
| chr2 | 199182620 | 199182754 |
| chr2 | 199293807 | 199293937 |
| chr2 | 199519617 | 199519741 |
| chr2 | 199532234 | 199532358 |
| chr2 | 199707560 | 199707714 |
| chr2 | 199861628 | 199861766 |
| chr2 | 200119959 | 200120217 |
| chr2 | 200248269 | 200248390 |
| chr2 | 200320031 | 200320360 |
| chr2 | 200542091 | 200542217 |
| chr2 | 200625066 | 200625192 |
| chr2 | 200850344 | 200850490 |
| chr2 | 200936309 | 200936433 |
| chr2 | 201035039 | 201035181 |
| chr2 | 201272647 | 201272779 |
| chr2 | 201330891 | 201331017 |
| chr2 | 201520508 | 201520646 |
| chr2 | 201656379 | 201656513 |
| chr2 | 201850814 | 201851069 |
| chr2 | 202032367 | 202032513 |
| chr2 | 202130250 | 202130392 |
| chr2 | 202295710 | 202295836 |
| chr2 | 202471150 | 202471298 |
| chr2 | 202551327 | 202551451 |
| chr2 | 202713685 | 202713813 |
| chr2 | 202951660 | 202951794 |
| chr2 | 203085161 | 203085307 |
| chr2 | 203250780 | 203250901 |
| chr2 | 203315572 | 203315696 |
| chr2 | 203524943 | 203525213 |
| chr2 | 203627344 | 203627496 |
| chr2 | 203843043 | 203843169 |
| chr2 | 203987480 | 203987604 |
| chr2 | 204129860 | 204129982 |
| chr2 | 204211808 | 204211934 |
| chr2 | 204460349 | 204460477 |
| chr2 | 204529869 | 204529997 |
| chr2 | 204683429 | 204683555 |
| chr2 | 204876984 | 204877128 |
| chr2 | 204967472 | 204967596 |
| chr2 | 205200287 | 205200419 |
| chr2 | 205260355 | 205260497 |
| chr2 | 205511252 | 205511396 |
| chr2 | 205536306 | 205536458 |
| chr2 | 205719309 | 205719439 |
| chr2 | 205924026 | 205924156 |
| chr2 | 205989962 | 205990088 |
| chr2 | 206259946 | 206260068 |
| chr2 | 206303834 | 206303970 |
| chr2 | 206557499 | 206557621 |
| chr2 | 206727976 | 206728124 |
| chr2 | 206866728 | 206866852 |
| chr2 | 206985637 | 206985781 |
| chr2 | 207083550 | 207083682 |
| chr2 | 207229731 | 207229855 |
| chr2 | 207356669 | 207356793 |
| chr2 | 207588667 | 207588807 |
| chr2 | 207771231 | 207771363 |
| chr2 | 207927751 | 207927879 |
| chr2 | 208046373 | 208046519 |
| chr2 | 208153623 | 208153745 |
| chr2 | 208340338 | 208340464 |
| chr2 | 208426215 | 208426339 |
| chr2 | 208536484 | 208536608 |
| chr2 | 208803470 | 208803644 |
| chr2 | 208962032 | 208962156 |
| chr2 | 209078445 | 209078714 |
| chr2 | 209131740 | 209131884 |
| chr2 | 209421688 | 209421818 |
| chr2 | 209484069 | 209484201 |
| chr2 | 209723126 | 209723280 |
| chr2 | 209849243 | 209849387 |
| chr2 | 209959974 | 209960118 |
| chr2 | 210088421 | 210088551 |
| chr2 | 210181008 | 210181134 |
| chr2 | 210430488 | 210430614 |
| chr2 | 210619508 | 210619656 |
| chr2 | 210722682 | 210722830 |
| chr2 | 210808815 | 210808959 |
| chr2 | 210933884 | 210934026 |
| chr2 | 211133092 | 211133236 |
| chr2 | 211262850 | 211262976 |
| chr2 | 211469067 | 211469201 |
| chr2 | 211595461 | 211595607 |
| chr2 | 211777654 | 211777776 |
| chr2 | 211862156 | 211862306 |
| chr2 | 212077188 | 212077312 |
| chr2 | 212231727 | 212231849 |
| chr2 | 212355418 | 212355552 |
| chr2 | 212446762 | 212446890 |
| chr2 | 212605816 | 212605940 |
| chr2 | 212799926 | 212800048 |
| chr2 | 212905294 | 212905536 |
| chr2 | 213045154 | 213045290 |
| chr2 | 213207831 | 213207977 |
| chr2 | 213421728 | 213421852 |
| chr2 | 213504646 | 213504772 |
| chr2 | 213766001 | 213766129 |
| chr2 | 213879514 | 213879648 |
| chr2 | 213937167 | 213937293 |
| chr2 | 214217636 | 214217788 |
| chr2 | 214326946 | 214327094 |
| chr2 | 214392813 | 214392939 |
| chr2 | 214561028 | 214561150 |
| chr2 | 214700716 | 214700838 |
| chr2 | 214831900 | 214832038 |
| chr2 | 215027365 | 215027495 |
| chr2 | 215168305 | 215168429 |
| chr2 | 215286201 | 215286329 |
| chr2 | 215563118 | 215563258 |
| chr2 | 215709807 | 215709941 |
| chr2 | 215749116 | 215749264 |
| chr2 | 215908804 | 215908928 |
| chr2 | 216063062 | 216063188 |
| chr2 | 216253364 | 216253516 |
| chr2 | 216463852 | 216463998 |
| chr2 | 216576229 | 216576367 |
| chr2 | 216725855 | 216725979 |
| chr2 | 216784298 | 216784444 |
| chr2 | 217041807 | 217041945 |
| chr2 | 217186677 | 217186805 |
| chr2 | 217241476 | 217241612 |
| chr2 | 217427965 | 217428089 |
| chr2 | 217545890 | 217546011 |
| chr2 | 217769570 | 217769692 |
| chr2 | 217940540 | 217940680 |
| chr2 | 218022479 | 218022657 |
| chr2 | 218266280 | 218266404 |
| chr2 | 218346369 | 218346537 |
| chr2 | 218449935 | 218450063 |
| chr2 | 218629217 | 218629353 |
| chr2 | 218811073 | 218811199 |
| chr2 | 218907108 | 218907236 |
| chr2 | 219112912 | 219113034 |
| chr2 | 219304777 | 219304913 |
| chr2 | 219389154 | 219389278 |
| chr2 | 219510284 | 219510408 |
| chr2 | 219750702 | 219750828 |
| chr2 | 219906626 | 219906752 |
| chr2 | 219987015 | 219987139 |
| chr2 | 220140706 | 220140834 |
| chr2 | 220315477 | 220315605 |
| chr2 | 220461792 | 220462054 |
| chr2 | 220626953 | 220627077 |
| chr2 | 220817056 | 220817204 |
| chr2 | 220969069 | 220969203 |
| chr2 | 221076969 | 221077091 |
| chr2 | 221254536 | 221254662 |
| chr2 | 221324291 | 221324417 |
| chr2 | 221484467 | 221484605 |
| chr2 | 221617870 | 221618014 |
| chr2 | 221731242 | 221731370 |
| chr2 | 221988881 | 221989005 |
| chr2 | 222169376 | 222169518 |
| chr2 | 222200790 | 222200926 |
| chr2 | 222362919 | 222363051 |
| chr2 | 222584104 | 222584373 |
| chr2 | 222748461 | 222748603 |
| chr2 | 222786076 | 222786218 |
| chr2 | 223075011 | 223075132 |
| chr2 | 223136927 | 223137049 |
| chr2 | 223297196 | 223297332 |
| chr2 | 223385902 | 223386028 |
| chr2 | 223570322 | 223570450 |
| chr2 | 223748490 | 223748628 |
| chr2 | 223893961 | 223894103 |
| chr2 | 224112453 | 224112577 |
| chr2 | 224262619 | 224262753 |
| chr2 | 224333644 | 224333786 |
| chr2 | 224530778 | 224530902 |
| chr2 | 224605286 | 224605424 |
| chr2 | 224847716 | 224847844 |
| chr2 | 224945445 | 224945593 |
| chr2 | 225078057 | 225078193 |
| chr2 | 225259294 | 225259442 |
| chr2 | 225347623 | 225347763 |
| chr2 | 225587391 | 225587519 |
| chr2 | 225655397 | 225655523 |
| chr2 | 225841425 | 225841553 |
| chr2 | 225956991 | 225957121 |
| chr2 | 226117608 | 226117744 |
| chr2 | 226234410 | 226234560 |
| chr2 | 226526197 | 226526343 |
| chr2 | 226551415 | 226551565 |
| chr2 | 226800833 | 226800957 |
| chr2 | 226867927 | 226868081 |
| chr2 | 227046012 | 227046148 |
| chr2 | 227159139 | 227159265 |
| chr2 | 227288232 | 227288368 |
| chr2 | 227482578 | 227482738 |
| chr2 | 227703059 | 227703205 |
| chr2 | 227821783 | 227821929 |
| chr2 | 228008675 | 228008803 |
| chr2 | 228136717 | 228136847 |
| chr2 | 228260671 | 228260807 |
| chr2 | 228423735 | 228423859 |
| chr2 | 228567929 | 228568053 |
| chr2 | 228772885 | 228773011 |
| chr2 | 228928642 | 228928774 |
| chr2 | 229038743 | 229038873 |
| chr2 | 229087134 | 229087258 |
| chr2 | 229239891 | 229240023 |
| chr2 | 229493806 | 229493942 |
| chr2 | 229663262 | 229663384 |
| chr2 | 229723388 | 229723512 |
| chr2 | 229936758 | 229936894 |
| chr2 | 230109502 | 230109630 |
| chr2 | 230132264 | 230132396 |
| chr2 | 230412040 | 230412176 |
| chr2 | 230468582 | 230468704 |
| chr2 | 230624855 | 230624977 |
| chr2 | 230752894 | 230753050 |
| chr2 | 230901569 | 230901701 |
| chr2 | 231174219 | 231174373 |
| chr2 | 231283623 | 231283747 |
| chr2 | 231384861 | 231385003 |
| chr2 | 231544327 | 231544449 |
| chr2 | 231646021 | 231646153 |
| chr2 | 231849193 | 231849315 |
| chr2 | 231992966 | 231993116 |
| chr2 | 232171137 | 232171283 |
| chr2 | 232256794 | 232256952 |
| chr2 | 232381155 | 232381281 |
| chr2 | 232609666 | 232609790 |
| chr2 | 232788438 | 232788570 |
| chr2 | 232929118 | 232929288 |
| chr2 | 233076330 | 233076454 |
| chr2 | 233166346 | 233166480 |
| chr2 | 233290890 | 233291014 |
| chr2 | 233486727 | 233486867 |
| chr2 | 233726118 | 233726242 |
| chr2 | 233825975 | 233826119 |
| chr2 | 234001286 | 234001480 |
| chr2 | 234055455 | 234055578 |
| chr2 | 234259426 | 234259552 |
| chr2 | 234422205 | 234422345 |
| chr2 | 234582916 | 234583130 |
| chr2 | 234681583 | 234681709 |
| chr2 | 234854846 | 234854982 |
| chr2 | 234943974 | 234944100 |
| chr2 | 235166719 | 235166849 |
| chr2 | 235266190 | 235266314 |
| chr2 | 235492413 | 235492555 |
| chr2 | 235575979 | 235576113 |
| chr2 | 235748077 | 235748205 |
| chr2 | 235873381 | 235873507 |
| chr2 | 236015346 | 236015470 |
| chr2 | 236242176 | 236242324 |
| chr2 | 236428931 | 236429053 |
| chr2 | 236530591 | 236530715 |
| chr2 | 236615574 | 236615710 |
| chr2 | 236825150 | 236825274 |
| chr2 | 236992957 | 236993103 |
| chr2 | 237063882 | 237064004 |
| chr2 | 237314695 | 237314819 |
| chr2 | 237462521 | 237462645 |
| chr2 | 237595685 | 237595839 |
| chr2 | 237691771 | 237691901 |
| chr2 | 237832280 | 237832430 |
| chr2 | 238051120 | 238051286 |
| chr2 | 238141883 | 238142025 |
| chr2 | 238295777 | 238295915 |
| chr2 | 238441697 | 238441839 |
| chr2 | 238542664 | 238542788 |
| chr2 | 238816536 | 238816662 |
| chr2 | 238912442 | 238912566 |
| chr2 | 239084073 | 239084207 |
| chr2 | 239144506 | 239144630 |
| chr2 | 239295640 | 239295778 |
| chr2 | 239497395 | 239497517 |
| chr2 | 239679985 | 239680113 |
| chr2 | 239739375 | 239739527 |
| chr2 | 240025180 | 240025301 |
| chr2 | 240163024 | 240163204 |
| chr2 | 240277144 | 240277549 |
| chr2 | 240374169 | 240374335 |
| chr2 | 240484513 | 240484635 |
| chr2 | 240739267 | 240739391 |
| chr2 | 240905938 | 240906076 |
| chr2 | 241020770 | 241020904 |
| chr2 | 241158344 | 241158494 |
| chr2 | 241291738 | 241291874 |
| chr2 | 241476987 | 241477125 |
| chr2 | 241653223 | 241653375 |
| chr2 | 241762507 | 241762667 |
| chr2 | 241968973 | 241969121 |
| chr2 | 242101410 | 242101560 |
| chr2 | 242214218 | 242214342 |
| chr2 | 242399443 | 242399579 |
| chr2 | 242490568 | 242490692 |
| chr2 | 242627862 | 242627986 |
| chr2 | 242854357 | 242854495 |
| chr2 | 242975773 | 242975903 |
| chr3 | 197826 | 197950 |
| chr3 | 275011 | 275135 |
| chr3 | 461887 | 462013 |
| chr3 | 553937 | 554073 |
| chr3 | 663845 | 664108 |
| chr3 | 930501 | 930625 |
| chr3 | 1046009 | 1046131 |
| chr3 | 1186688 | 1186838 |
| chr3 | 1393589 | 1393733 |
| chr3 | 1557995 | 1558123 |
| chr3 | 1605844 | 1605984 |
| chr3 | 1851952 | 1852086 |
| chr3 | 1988036 | 1988190 |
| chr3 | 2155208 | 2155346 |
| chr3 | 2309289 | 2309437 |
| chr3 | 2412795 | 2412917 |
| chr3 | 2497477 | 2497609 |
| chr3 | 2667115 | 2667245 |
| chr3 | 2769304 | 2769434 |
| chr3 | 2932315 | 2932449 |
| chr3 | 3078529 | 3078651 |
| chr3 | 3213739 | 3213873 |
| chr3 | 3472550 | 3472674 |
| chr3 | 3527611 | 3527733 |
| chr3 | 3699786 | 3699924 |
| chr3 | 3896607 | 3896737 |
| chr3 | 4028618 | 4028756 |
| chr3 | 4239909 | 4240041 |
| chr3 | 4277304 | 4277442 |
| chr3 | 4491179 | 4491325 |
| chr3 | 4647292 | 4647428 |
| chr3 | 4756786 | 4756908 |
| chr3 | 4982272 | 4982406 |
| chr3 | 5085747 | 5085869 |
| chr3 | 5231990 | 5232162 |
| chr3 | 5352775 | 5352899 |
| chr3 | 5547890 | 5548014 |
| chr3 | 5617078 | 5617244 |
| chr3 | 5882629 | 5882765 |
| chr3 | 5962679 | 5962817 |
| chr3 | 6126280 | 6126416 |
| chr3 | 6342792 | 6342920 |
| chr3 | 6408646 | 6408768 |
| chr3 | 6646434 | 6646560 |
| chr3 | 6700462 | 6700586 |
| chr3 | 6859593 | 6859719 |
| chr3 | 7008484 | 7008610 |
| chr3 | 7231772 | 7231920 |
| chr3 | 7330125 | 7330253 |
| chr3 | 7470173 | 7470329 |
| chr3 | 7648699 | 7648853 |
| chr3 | 7837693 | 7837831 |
| chr3 | 7987410 | 7987550 |
| chr3 | 8117912 | 8118034 |
| chr3 | 8185530 | 8185658 |
| chr3 | 8413126 | 8413262 |
| chr3 | 8494078 | 8494218 |
| chr3 | 8744964 | 8745096 |
| chr3 | 8858269 | 8858421 |
| chr3 | 9041211 | 9041383 |
| chr3 | 9106920 | 9107046 |
| chr3 | 9264546 | 9264686 |
| chr3 | 9428232 | 9428354 |
| chr3 | 9652230 | 9652354 |
| chr3 | 9789785 | 9789913 |
| chr3 | 9871319 | 9871449 |
| chr3 | 10024621 | 10024757 |
| chr3 | 10243214 | 10243366 |
| chr3 | 10358036 | 10358168 |
| chr3 | 10463479 | 10463603 |
| chr3 | 10563025 | 10563159 |
| chr3 | 10753781 | 10753905 |
| chr3 | 10932539 | 10932661 |
| chr3 | 11153539 | 11153675 |
| chr3 | 11254025 | 11254149 |
| chr3 | 11365394 | 11365524 |
| chr3 | 11483711 | 11483845 |
| chr3 | 11644060 | 11644200 |
| chr3 | 11767505 | 11767627 |
| chr3 | 12056779 | 12056921 |
| chr3 | 12102767 | 12102909 |
| chr3 | 12353112 | 12353236 |
| chr3 | 12465183 | 12465313 |
| chr3 | 12614441 | 12614687 |
| chr3 | 12671756 | 12672005 |
| chr3 | 12933363 | 12933489 |
| chr3 | 13090919 | 13091043 |
| chr3 | 13157014 | 13157138 |
| chr3 | 13404709 | 13404837 |
| chr3 | 13499515 | 13499639 |
| chr3 | 13665376 | 13665498 |
| chr3 | 13849627 | 13849753 |
| chr3 | 13971457 | 13971581 |
| chr3 | 14018719 | 14018851 |
| chr3 | 14265902 | 14266024 |
| chr3 | 14331088 | 14331226 |
| chr3 | 14476421 | 14476585 |
| chr3 | 14721176 | 14721326 |
| chr3 | 14876177 | 14876301 |
| chr3 | 14977509 | 14977633 |
| chr3 | 15155069 | 15155191 |
| chr3 | 15175998 | 15176132 |
| chr3 | 15177469 | 15177589 |
| chr3 | 15314939 | 15315079 |
| chr3 | 15464252 | 15464390 |
| chr3 | 15647938 | 15648074 |
| chr3 | 15706038 | 15706176 |
| chr3 | 15821069 | 15821191 |
| chr3 | 15995310 | 15995454 |
| chr3 | 16162527 | 16162651 |
| chr3 | 16282614 | 16282738 |
| chr3 | 16428407 | 16428529 |
| chr3 | 16595528 | 16595664 |
| chr3 | 16747331 | 16747475 |
| chr3 | 16990692 | 16990824 |
| chr3 | 17045532 | 17045662 |
| chr3 | 17249042 | 17249166 |
| chr3 | 17378465 | 17378593 |
| chr3 | 17486553 | 17486697 |
| chr3 | 17743208 | 17743336 |
| chr3 | 17804942 | 17805063 |
| chr3 | 17987358 | 17987480 |
| chr3 | 18114766 | 18115046 |
| chr3 | 18227369 | 18227493 |
| chr3 | 18372874 | 18373006 |
| chr3 | 18577122 | 18577262 |
| chr3 | 18711660 | 18711784 |
| chr3 | 18931722 | 18931846 |
| chr3 | 19104599 | 19104854 |
| chr3 | 19214155 | 19214322 |
| chr3 | 19268593 | 19268786 |
| chr3 | 19509277 | 19509398 |
| chr3 | 19605087 | 19605225 |
| chr3 | 19740589 | 19740717 |
| chr3 | 20003088 | 20003240 |
| chr3 | 20052812 | 20052950 |
| chr3 | 20239349 | 20239473 |
| chr3 | 20416986 | 20417110 |
| chr3 | 20465130 | 20465256 |
| chr3 | 20704822 | 20704964 |
| chr3 | 20776138 | 20776284 |
| chr3 | 21008644 | 21008786 |
| chr3 | 21095866 | 21096014 |
| chr3 | 21333794 | 21333918 |
| chr3 | 21396100 | 21396230 |
| chr3 | 21582273 | 21582397 |
| chr3 | 21666836 | 21666968 |
| chr3 | 21837630 | 21837762 |
| chr3 | 22110400 | 22110524 |
| chr3 | 22181958 | 22182112 |
| chr3 | 22388286 | 22388412 |
| chr3 | 22470417 | 22470541 |
| chr3 | 22631541 | 22631667 |
| chr3 | 22774820 | 22774952 |
| chr3 | 22920190 | 22920316 |
| chr3 | 23054464 | 23054586 |
| chr3 | 23226499 | 23226633 |
| chr3 | 23321573 | 23321695 |
| chr3 | 23576881 | 23577007 |
| chr3 | 23635347 | 23635477 |
| chr3 | 23764516 | 23764652 |
| chr3 | 23961345 | 23961471 |
| chr3 | 24163900 | 24164050 |
| chr3 | 24225814 | 24225942 |
| chr3 | 24405589 | 24405725 |
| chr3 | 24639487 | 24639625 |
| chr3 | 24692171 | 24692317 |
| chr3 | 24887944 | 24888080 |
| chr3 | 25077734 | 25077860 |
| chr3 | 25248183 | 25248331 |
| chr3 | 25347348 | 25347482 |
| chr3 | 25440061 | 25440187 |
| chr3 | 25645434 | 25645562 |
| chr3 | 25818291 | 25818415 |
| chr3 | 25979708 | 25979829 |
| chr3 | 26031968 | 26032224 |
| chr3 | 26212866 | 26212987 |
| chr3 | 26387795 | 26387947 |
| chr3 | 26505811 | 26505933 |
| chr3 | 26639103 | 26639227 |
| chr3 | 26899063 | 26899187 |
| chr3 | 27050741 | 27050867 |
| chr3 | 27138504 | 27138634 |
| chr3 | 27348862 | 27348990 |
| chr3 | 27456539 | 27456663 |
| chr3 | 27648300 | 27648458 |
| chr3 | 27707845 | 27707989 |
| chr3 | 27824028 | 27824152 |
| chr3 | 28047418 | 28047568 |
| chr3 | 28181535 | 28181661 |
| chr3 | 28332009 | 28332135 |
| chr3 | 28421967 | 28422099 |
| chr3 | 28624257 | 28624381 |
| chr3 | 28766273 | 28766399 |
| chr3 | 28928824 | 28928948 |
| chr3 | 29068702 | 29068836 |
| chr3 | 29283192 | 29283314 |
| chr3 | 29394637 | 29394761 |
| chr3 | 29476817 | 29476949 |
| chr3 | 29685178 | 29685316 |
| chr3 | 29841391 | 29841523 |
| chr3 | 29986888 | 29987020 |
| chr3 | 30136510 | 30136648 |
| chr3 | 30360802 | 30360924 |
| chr3 | 30449200 | 30449356 |
| chr3 | 30661120 | 30661266 |
| chr3 | 30717609 | 30717731 |
| chr3 | 30828817 | 30828959 |
| chr3 | 31016363 | 31016495 |
| chr3 | 31235743 | 31235865 |
| chr3 | 31370735 | 31370871 |
| chr3 | 31510224 | 31510346 |
| chr3 | 31604644 | 31604768 |
| chr3 | 31838132 | 31838272 |
| chr3 | 31967370 | 31967502 |
| chr3 | 32035563 | 32035707 |
| chr3 | 32239207 | 32239331 |
| chr3 | 32451305 | 32451433 |
| chr3 | 32465342 | 32465464 |
| chr3 | 32756587 | 32756858 |
| chr3 | 32900165 | 32900301 |
| chr3 | 33019286 | 33019408 |
| chr3 | 33142022 | 33142146 |
| chr3 | 33221035 | 33221201 |
| chr3 | 33449174 | 33449306 |
| chr3 | 33574628 | 33574762 |
| chr3 | 33693704 | 33693832 |
| chr3 | 33832622 | 33832762 |
| chr3 | 34068905 | 34069029 |
| chr3 | 34238929 | 34239055 |
| chr3 | 34264785 | 34264911 |
| chr3 | 34538472 | 34538594 |
| chr3 | 34623357 | 34623497 |
| chr3 | 34812014 | 34812136 |
| chr3 | 34959323 | 34959449 |
| chr3 | 35084893 | 35085015 |
| chr3 | 35229128 | 35229250 |
| chr3 | 35397419 | 35397543 |
| chr3 | 35543390 | 35543512 |
| chr3 | 35619501 | 35619647 |
| chr3 | 35891649 | 35891773 |
| chr3 | 36012889 | 36013021 |
| chr3 | 36117436 | 36117574 |
| chr3 | 36212786 | 36212932 |
| chr3 | 36459164 | 36459300 |
| chr3 | 36621140 | 36621276 |
| chr3 | 36776953 | 36777085 |
| chr3 | 36960582 | 36960708 |
| chr3 | 37038681 | 37038827 |
| chr3 | 37143346 | 37143474 |
| chr3 | 37298205 | 37298329 |
| chr3 | 37525969 | 37526093 |
| chr3 | 37619294 | 37619416 |
| chr3 | 37784688 | 37784810 |
| chr3 | 37908791 | 37908915 |
| chr3 | 38144785 | 38144915 |
| chr3 | 38260985 | 38261125 |
| chr3 | 38327208 | 38327368 |
| chr3 | 38553565 | 38553701 |
| chr3 | 38750285 | 38750411 |
| chr3 | 38832955 | 38833081 |
| chr3 | 38920119 | 38920269 |
| chr3 | 39208441 | 39208577 |
| chr3 | 39357247 | 39357371 |
| chr3 | 39441780 | 39441906 |
| chr3 | 39549713 | 39549859 |
| chr3 | 39768053 | 39768191 |
| chr3 | 39955224 | 39955354 |
| chr3 | 39975522 | 39975660 |
| chr3 | 40143728 | 40143878 |
| chr3 | 40329200 | 40329350 |
| chr3 | 40535727 | 40535861 |
| chr3 | 40565524 | 40565682 |
| chr3 | 40750618 | 40750885 |
| chr3 | 40957881 | 40958009 |
| chr3 | 41042770 | 41042896 |
| chr3 | 41225505 | 41225631 |
| chr3 | 41438212 | 41438336 |
| chr3 | 41599448 | 41599582 |
| chr3 | 41719900 | 41720092 |
| chr3 | 41878587 | 41878709 |
| chr3 | 42019529 | 42019653 |
| chr3 | 42077032 | 42077156 |
| chr3 | 42293604 | 42293728 |
| chr3 | 42396045 | 42396183 |
| chr3 | 42643335 | 42643463 |
| chr3 | 42719962 | 42720110 |
| chr3 | 42930326 | 42930462 |
| chr3 | 43048105 | 43048247 |
| chr3 | 43175820 | 43175972 |
| chr3 | 43387866 | 43388008 |
| chr3 | 43433272 | 43433398 |
| chr3 | 43659385 | 43659523 |
| chr3 | 43778180 | 43778447 |
| chr3 | 43914764 | 43914890 |
| chr3 | 44046945 | 44047069 |
| chr3 | 44196598 | 44196748 |
| chr3 | 44450041 | 44450165 |
| chr3 | 44520906 | 44521030 |
| chr3 | 44704019 | 44704141 |
| chr3 | 44871809 | 44871951 |
| chr3 | 44912099 | 44912223 |
| chr3 | 45203808 | 45203980 |
| chr3 | 45340406 | 45340550 |
| chr3 | 45509668 | 45509802 |
| chr3 | 45572199 | 45572327 |
| chr3 | 45796393 | 45796517 |
| chr3 | 45885282 | 45885422 |
| chr3 | 46053718 | 46053848 |
| chr3 | 46149981 | 46150133 |
| chr3 | 46346584 | 46346726 |
| chr3 | 46420098 | 46420258 |
| chr3 | 46615610 | 46615853 |
| chr3 | 46858050 | 46858293 |
| chr3 | 46988477 | 46988619 |
| chr3 | 47138618 | 47138762 |
| chr3 | 47214064 | 47214192 |
| chr3 | 47424970 | 47425110 |
| chr3 | 47564415 | 47564577 |
| chr3 | 47688087 | 47688217 |
| chr3 | 47803745 | 47803869 |
| chr3 | 48056327 | 48056459 |
| chr3 | 48148616 | 48148760 |
| chr3 | 48232228 | 48232502 |
| chr3 | 48370544 | 48370666 |
| chr3 | 48645912 | 48646089 |
| chr3 | 48754924 | 48755210 |
| chr3 | 48864522 | 48864763 |
| chr3 | 49057226 | 49057470 |
| chr3 | 49188473 | 49188664 |
| chr3 | 49409620 | 49409774 |
| chr3 | 49423920 | 49424046 |
| chr3 | 49652066 | 49652204 |
| chr3 | 49729235 | 49729359 |
| chr3 | 49819064 | 49819190 |
| chr3 | 50004123 | 50004267 |
| chr3 | 50028327 | 50028473 |
| chr3 | 50306994 | 50307138 |
| chr3 | 50420995 | 50421141 |
| chr3 | 50581174 | 50581300 |
| chr3 | 50631724 | 50631848 |
| chr3 | 50772582 | 50772706 |
| chr3 | 50928433 | 50928707 |
| chr3 | 51114269 | 51114423 |
| chr3 | 51311526 | 51311648 |
| chr3 | 51381109 | 51381233 |
| chr3 | 51545093 | 51545245 |
| chr3 | 51695785 | 51695931 |
| chr3 | 51816626 | 51816766 |
| chr3 | 52030901 | 52031173 |
| chr3 | 52159438 | 52159564 |
| chr3 | 52322727 | 52322883 |
| chr3 | 52414812 | 52415075 |
| chr3 | 52593068 | 52593198 |
| chr3 | 52742323 | 52742465 |
| chr3 | 53007909 | 53008037 |
| chr3 | 53120008 | 53120154 |
| chr3 | 53216654 | 53216778 |
| chr3 | 53332413 | 53332561 |
| chr3 | 53544788 | 53544942 |
| chr3 | 53641879 | 53642017 |
| chr3 | 53765392 | 53765534 |
| chr3 | 54024009 | 54024155 |
| chr3 | 54086529 | 54086659 |
| chr3 | 54278924 | 54279062 |
| chr3 | 54462915 | 54463041 |
| chr3 | 54570150 | 54570284 |
| chr3 | 54807234 | 54807364 |
| chr3 | 54861186 | 54861326 |
| chr3 | 55105312 | 55105448 |
| chr3 | 55200978 | 55201122 |
| chr3 | 55373618 | 55373768 |
| chr3 | 55550990 | 55551112 |
| chr3 | 55653732 | 55653872 |
| chr3 | 55837018 | 55837164 |
| chr3 | 55876692 | 55876816 |
| chr3 | 56088942 | 56089064 |
| chr3 | 56207944 | 56208068 |
| chr3 | 56429873 | 56429997 |
| chr3 | 56471074 | 56471208 |
| chr3 | 56709725 | 56709867 |
| chr3 | 56894829 | 56894953 |
| chr3 | 56990514 | 56990636 |
| chr3 | 57171822 | 57171950 |
| chr3 | 57263318 | 57263482 |
| chr3 | 57382533 | 57382657 |
| chr3 | 57533572 | 57533714 |
| chr3 | 57801662 | 57801802 |
| chr3 | 57888328 | 57888452 |
| chr3 | 58009294 | 58009426 |
| chr3 | 58228133 | 58228257 |
| chr3 | 58335527 | 58335655 |
| chr3 | 58419964 | 58420102 |
| chr3 | 58673148 | 58673280 |
| chr3 | 58729719 | 58729845 |
| chr3 | 58903847 | 58904089 |
| chr3 | 59076136 | 59076262 |
| chr3 | 59240416 | 59240558 |
| chr3 | 59317286 | 59317412 |
| chr3 | 59549948 | 59550070 |
| chr3 | 59643553 | 59643679 |
| chr3 | 59844712 | 59844852 |
| chr3 | 59958926 | 59959068 |
| chr3 | 60112557 | 60112693 |
| chr3 | 60321046 | 60321170 |
| chr3 | 60392998 | 60393140 |
| chr3 | 60512805 | 60512945 |
| chr3 | 60754678 | 60754808 |
| chr3 | 60833421 | 60833543 |
| chr3 | 61082097 | 61082225 |
| chr3 | 61227152 | 61227276 |
| chr3 | 61289495 | 61289621 |
| chr3 | 61438156 | 61438286 |
| chr3 | 61620717 | 61620855 |
| chr3 | 61774022 | 61774148 |
| chr3 | 61949819 | 61949969 |
| chr3 | 62073151 | 62073283 |
| chr3 | 62281368 | 62281498 |
| chr3 | 62411513 | 62411639 |
| chr3 | 62478698 | 62478824 |
| chr3 | 62645224 | 62645362 |
| chr3 | 62789492 | 62789630 |
| chr3 | 62973397 | 62973521 |
| chr3 | 63185712 | 63185836 |
| chr3 | 63349391 | 63349539 |
| chr3 | 63407635 | 63407759 |
| chr3 | 63653901 | 63654031 |
| chr3 | 63788050 | 63788174 |
| chr3 | 63951693 | 63951853 |
| chr3 | 64031687 | 64031813 |
| chr3 | 64232445 | 64232569 |
| chr3 | 64396720 | 64396850 |
| chr3 | 64480816 | 64480938 |
| chr3 | 64575573 | 64575717 |
| chr3 | 64829432 | 64829558 |
| chr3 | 64953216 | 64953346 |
| chr3 | 65142424 | 65142550 |
| chr3 | 65188417 | 65188541 |
| chr3 | 65318318 | 65318470 |
| chr3 | 65595821 | 65595955 |
| chr3 | 65752715 | 65752837 |
| chr3 | 65812313 | 65812487 |
| chr3 | 65944000 | 65944152 |
| chr3 | 66169609 | 66169748 |
| chr3 | 66299034 | 66299304 |
| chr3 | 66506334 | 66506458 |
| chr3 | 66638834 | 66638968 |
| chr3 | 66790142 | 66790266 |
| chr3 | 66858520 | 66858658 |
| chr3 | 67010997 | 67011125 |
| chr3 | 67211794 | 67211946 |
| chr3 | 67335184 | 67335308 |
| chr3 | 67426782 | 67426912 |
| chr3 | 67608204 | 67608354 |
| chr3 | 67771132 | 67771262 |
| chr3 | 67971477 | 67971601 |
| chr3 | 68018202 | 68018324 |
| chr3 | 68272826 | 68272982 |
| chr3 | 68369352 | 68369480 |
| chr3 | 68511948 | 68512078 |
| chr3 | 68730947 | 68731085 |
| chr3 | 68777544 | 68777678 |
| chr3 | 69021835 | 69021959 |
| chr3 | 69102910 | 69103042 |
| chr3 | 69332673 | 69332797 |
| chr3 | 69413638 | 69413776 |
| chr3 | 69622545 | 69622673 |
| chr3 | 69796440 | 69796580 |
| chr3 | 69897018 | 69897142 |
| chr3 | 69974298 | 69974421 |
| chr3 | 70245961 | 70246111 |
| chr3 | 70281052 | 70281204 |
| chr3 | 70521947 | 70522105 |
| chr3 | 70609281 | 70609421 |
| chr3 | 70751021 | 70751153 |
| chr3 | 70967040 | 70967170 |
| chr3 | 71039546 | 71039670 |
| chr3 | 71161336 | 71161458 |
| chr3 | 71397860 | 71397998 |
| chr3 | 71578370 | 71578498 |
| chr3 | 71611544 | 71611668 |
| chr3 | 71898583 | 71898709 |
| chr3 | 72058851 | 72058983 |
| chr3 | 72132259 | 72132435 |
| chr3 | 72242038 | 72242178 |
| chr3 | 72424389 | 72424541 |
| chr3 | 72653354 | 72653502 |
| chr3 | 72801205 | 72801377 |
| chr3 | 72833843 | 72834088 |
| chr3 | 72995531 | 72995655 |
| chr3 | 73232405 | 73232531 |
| chr3 | 73293472 | 73293614 |
| chr3 | 73480895 | 73481019 |
| chr3 | 73573301 | 73573437 |
| chr3 | 73737305 | 73737431 |
| chr3 | 73865270 | 73865395 |
| chr3 | 74157091 | 74157245 |
| chr3 | 74263407 | 74263541 |
| chr3 | 74371613 | 74371737 |
| chr3 | 74532527 | 74532649 |
| chr3 | 74688395 | 74688535 |
| chr3 | 74841566 | 74841692 |
| chr3 | 74989905 | 74990035 |
| chr3 | 75062684 | 75062840 |
| chr3 | 75277813 | 75277949 |
| chr3 | 75386754 | 75387010 |
| chr3 | 75430705 | 75430825 |
| chr3 | 75518412 | 75518534 |
| chr3 | 75518553 | 75518672 |
| chr3 | 75595962 | 75596082 |
| chr3 | 75598852 | 75599007 |
| chr3 | 75775678 | 75775820 |
| chr3 | 75813773 | 75813917 |
| chr3 | 75876975 | 75877097 |
| chr3 | 76022834 | 76022958 |
| chr3 | 76254638 | 76254816 |
| chr3 | 76328944 | 76329070 |
| chr3 | 76425471 | 76425615 |
| chr3 | 76615027 | 76615173 |
| chr3 | 76827832 | 76827954 |
| chr3 | 76969441 | 76969565 |
| chr3 | 77123330 | 77123454 |
| chr3 | 77246675 | 77246799 |
| chr3 | 77383414 | 77383538 |
| chr3 | 77581221 | 77581345 |
| chr3 | 77630225 | 77630353 |
| chr3 | 77801629 | 77801769 |
| chr3 | 77976521 | 77976675 |
| chr3 | 78065821 | 78065965 |
| chr3 | 78277095 | 78277219 |
| chr3 | 78427937 | 78428065 |
| chr3 | 78652084 | 78652214 |
| chr3 | 78786069 | 78786213 |
| chr3 | 78868165 | 78868305 |
| chr3 | 78990517 | 78990641 |
| chr3 | 79137162 | 79137286 |
| chr3 | 79354113 | 79354235 |
| chr3 | 79426776 | 79426902 |
| chr3 | 79685556 | 79685678 |
| chr3 | 79784050 | 79784172 |
| chr3 | 79987658 | 79987788 |
| chr3 | 80147921 | 80148043 |
| chr3 | 80218508 | 80218652 |
| chr3 | 80404338 | 80404462 |
| chr3 | 80492799 | 80492929 |
| chr3 | 80673213 | 80673351 |
| chr3 | 80845543 | 80845685 |
| chr3 | 80940101 | 80940225 |
| chr3 | 81128802 | 81128936 |
| chr3 | 81339862 | 81339990 |
| chr3 | 81456742 | 81456868 |
| chr3 | 81614391 | 81614525 |
| chr3 | 81724404 | 81724536 |
| chr3 | 81891941 | 81892065 |
| chr3 | 81982846 | 81982970 |
| chr3 | 82257603 | 82257727 |
| chr3 | 82365490 | 82365614 |
| chr3 | 82436879 | 82437013 |
| chr3 | 82648096 | 82648234 |
| chr3 | 82735027 | 82735179 |
| chr3 | 82868111 | 82868368 |
| chr3 | 83053605 | 83053731 |
| chr3 | 83251345 | 83251495 |
| chr3 | 83458530 | 83458656 |
| chr3 | 83496922 | 83497070 |
| chr3 | 83647526 | 83647654 |
| chr3 | 83764583 | 83764711 |
| chr3 | 83970214 | 83970340 |
| chr3 | 84089997 | 84090127 |
| chr3 | 84344452 | 84344574 |
| chr3 | 84509245 | 84509393 |
| chr3 | 84539057 | 84539179 |
| chr3 | 84732674 | 84732810 |
| chr3 | 84947088 | 84947230 |
| chr3 | 84993323 | 84993451 |
| chr3 | 85151776 | 85151898 |
| chr3 | 85303988 | 85304110 |
| chr3 | 85498953 | 85499091 |
| chr3 | 85623191 | 85623315 |
| chr3 | 85781442 | 85781566 |
| chr3 | 85873820 | 85873954 |
| chr3 | 86118164 | 86118294 |
| chr3 | 86240804 | 86240930 |
| chr3 | 86362873 | 86363001 |
| chr3 | 86560567 | 86560689 |
| chr3 | 86689591 | 86689715 |
| chr3 | 86778440 | 86778564 |
| chr3 | 86927536 | 86927662 |
| chr3 | 87203190 | 87203314 |
| chr3 | 87223979 | 87224121 |
| chr3 | 87485963 | 87486085 |
| chr3 | 87520852 | 87520978 |
| chr3 | 87800121 | 87800247 |
| chr3 | 87864145 | 87864311 |
| chr3 | 87998890 | 87999014 |
| chr3 | 88145876 | 88146000 |
| chr3 | 88352035 | 88352191 |
| chr3 | 88472886 | 88473008 |
| chr3 | 88677921 | 88678059 |
| chr3 | 88846383 | 88846509 |
| chr3 | 88902378 | 88902504 |
| chr3 | 89049836 | 89049966 |
| chr3 | 89275858 | 89275990 |
| chr3 | 89394481 | 89394625 |
| chr3 | 89482174 | 89482322 |
| chr3 | 89741672 | 89741950 |
| chr3 | 89888024 | 89888213 |
| chr3 | 90056326 | 90056462 |
| chr3 | 90102960 | 90103084 |
| chr3 | 90253679 | 90253846 |
| chr3 | 93573817 | 93573938 |
| chr3 | 93730234 | 93730378 |
| chr3 | 93903536 | 93903686 |
| chr3 | 93991434 | 93991574 |
| chr3 | 94193339 | 94193473 |
| chr3 | 94305015 | 94305155 |
| chr3 | 94502845 | 94502985 |
| chr3 | 94563813 | 94563947 |
| chr3 | 94859772 | 94859908 |
| chr3 | 94913222 | 94913372 |
| chr3 | 95056370 | 95056500 |
| chr3 | 95270761 | 95270897 |
| chr3 | 95389451 | 95389591 |
| chr3 | 95558004 | 95558130 |
| chr3 | 95644170 | 95644304 |
| chr3 | 95818592 | 95818716 |
| chr3 | 96061190 | 96061322 |
| chr3 | 96090078 | 96090212 |
| chr3 | 96302791 | 96302920 |
| chr3 | 96489204 | 96489336 |
| chr3 | 96576270 | 96576398 |
| chr3 | 96667677 | 96667807 |
| chr3 | 96887898 | 96888153 |
| chr3 | 97103229 | 97103365 |
| chr3 | 97195702 | 97195826 |
| chr3 | 97305228 | 97305354 |
| chr3 | 97527175 | 97527303 |
| chr3 | 97635289 | 97635413 |
| chr3 | 97769691 | 97769829 |
| chr3 | 97862573 | 97862699 |
| chr3 | 98047127 | 98047249 |
| chr3 | 98207466 | 98207602 |
| chr3 | 98319605 | 98319729 |
| chr3 | 98479650 | 98479774 |
| chr3 | 98663963 | 98664115 |
| chr3 | 98769869 | 98770005 |
| chr3 | 98930367 | 98930505 |
| chr3 | 99107609 | 99107731 |
| chr3 | 99288552 | 99288680 |
| chr3 | 99428086 | 99428236 |
| chr3 | 99578046 | 99578188 |
| chr3 | 99757995 | 99758117 |
| chr3 | 99826890 | 99827014 |
| chr3 | 99962883 | 99963013 |
| chr3 | 100214638 | 100214792 |
| chr3 | 100357601 | 100357757 |
| chr3 | 100455059 | 100455189 |
| chr3 | 100575691 | 100575829 |
| chr3 | 100843059 | 100843197 |
| chr3 | 100921826 | 100921960 |
| chr3 | 101020825 | 101020953 |
| chr3 | 101307654 | 101307776 |
| chr3 | 101396443 | 101396573 |
| chr3 | 101534260 | 101534402 |
| chr3 | 101705727 | 101705857 |
| chr3 | 101904505 | 101904633 |
| chr3 | 102040984 | 102041126 |
| chr3 | 102141379 | 102141620 |
| chr3 | 102238489 | 102238613 |
| chr3 | 102438731 | 102438855 |
| chr3 | 102563729 | 102563861 |
| chr3 | 102707861 | 102707983 |
| chr3 | 102918309 | 102918453 |
| chr3 | 103053473 | 103053613 |
| chr3 | 103157927 | 103158055 |
| chr3 | 103271407 | 103271551 |
| chr3 | 103537480 | 103537604 |
| chr3 | 103594778 | 103594918 |
| chr3 | 103762366 | 103762508 |
| chr3 | 103961020 | 103961144 |
| chr3 | 104057666 | 104057810 |
| chr3 | 104300187 | 104300327 |
| chr3 | 104322322 | 104322462 |
| chr3 | 104590388 | 104590512 |
| chr3 | 104722024 | 104722202 |
| chr3 | 104853097 | 104853233 |
| chr3 | 105055364 | 105055488 |
| chr3 | 105151973 | 105152133 |
| chr3 | 105216364 | 105216496 |
| chr3 | 105441468 | 105441596 |
| chr3 | 105643761 | 105643913 |
| chr3 | 105800928 | 105801056 |
| chr3 | 105959172 | 105959296 |
| chr3 | 105996731 | 105996855 |
| chr3 | 106138406 | 106138530 |
| chr3 | 106283680 | 106283826 |
| chr3 | 106528570 | 106528700 |
| chr3 | 106693173 | 106693297 |
| chr3 | 106780058 | 106780194 |
| chr3 | 106981649 | 106981777 |
| chr3 | 107022443 | 107022569 |
| chr3 | 107270408 | 107270540 |
| chr3 | 107390517 | 107390641 |
| chr3 | 107479702 | 107479836 |
| chr3 | 107670584 | 107670708 |
| chr3 | 107765213 | 107765335 |
| chr3 | 107970070 | 107970208 |
| chr3 | 108163116 | 108163248 |
| chr3 | 108265468 | 108265590 |
| chr3 | 108459545 | 108459681 |
| chr3 | 108621522 | 108621646 |
| chr3 | 108670562 | 108670688 |
| chr3 | 108824963 | 108825084 |
| chr3 | 109042784 | 109042920 |
| chr3 | 109151621 | 109151759 |
| chr3 | 109332205 | 109332329 |
| chr3 | 109484230 | 109484382 |
| chr3 | 109672210 | 109672364 |
| chr3 | 109726966 | 109727120 |
| chr3 | 109983437 | 109983559 |
| chr3 | 110069779 | 110069937 |
| chr3 | 110213123 | 110213257 |
| chr3 | 110457478 | 110457604 |
| chr3 | 110558624 | 110558746 |
| chr3 | 110677100 | 110677252 |
| chr3 | 110802985 | 110803113 |
| chr3 | 110984212 | 110984346 |
| chr3 | 111131446 | 111131596 |
| chr3 | 111286081 | 111286205 |
| chr3 | 111482119 | 111482245 |
| chr3 | 111527199 | 111527321 |
| chr3 | 111696733 | 111696857 |
| chr3 | 111826207 | 111826329 |
| chr3 | 112039685 | 112039845 |
| chr3 | 112182688 | 112182810 |
| chr3 | 112287672 | 112287796 |
| chr3 | 112513312 | 112513434 |
| chr3 | 112575727 | 112575871 |
| chr3 | 112860714 | 112860838 |
| chr3 | 112959120 | 112959258 |
| chr3 | 113137990 | 113138114 |
| chr3 | 113282043 | 113282179 |
| chr3 | 113443999 | 113444121 |
| chr3 | 113473700 | 113473838 |
| chr3 | 113717630 | 113717782 |
| chr3 | 113785782 | 113785906 |
| chr3 | 113937909 | 113938061 |
| chr3 | 114183574 | 114183696 |
| chr3 | 114324682 | 114324924 |
| chr3 | 114368131 | 114368281 |
| chr3 | 114651556 | 114651836 |
| chr3 | 114714192 | 114714324 |
| chr3 | 114872703 | 114872977 |
| chr3 | 114983724 | 114983860 |
| chr3 | 115239349 | 115239477 |
| chr3 | 115308483 | 115308605 |
| chr3 | 115490063 | 115490207 |
| chr3 | 115596850 | 115596972 |
| chr3 | 115776509 | 115776649 |
| chr3 | 115965753 | 115965899 |
| chr3 | 116072885 | 116073029 |
| chr3 | 116223276 | 116223420 |
| chr3 | 116459291 | 116459425 |
| chr3 | 116483260 | 116483384 |
| chr3 | 116729762 | 116729898 |
| chr3 | 116814852 | 116814982 |
| chr3 | 117027456 | 117027596 |
| chr3 | 117066556 | 117066714 |
| chr3 | 117233123 | 117233265 |
| chr3 | 117493057 | 117493181 |
| chr3 | 117637241 | 117637379 |
| chr3 | 117718160 | 117718288 |
| chr3 | 117852862 | 117853014 |
| chr3 | 118059538 | 118059664 |
| chr3 | 118165989 | 118166111 |
| chr3 | 118392779 | 118392907 |
| chr3 | 118412172 | 118412316 |
| chr3 | 118657024 | 118657170 |
| chr3 | 118822557 | 118822703 |
| chr3 | 119006227 | 119006355 |
| chr3 | 119042921 | 119043049 |
| chr3 | 119269024 | 119269162 |
| chr3 | 119413697 | 119413829 |
| chr3 | 119491541 | 119491663 |
| chr3 | 119668239 | 119668365 |
| chr3 | 119811516 | 119811654 |
| chr3 | 119915347 | 119915495 |
| chr3 | 120064414 | 120064586 |
| chr3 | 120329058 | 120329182 |
| chr3 | 120489868 | 120489996 |
| chr3 | 120630261 | 120630395 |
| chr3 | 120727487 | 120727621 |
| chr3 | 120950008 | 120950166 |
| chr3 | 121005925 | 121006049 |
| chr3 | 121167542 | 121167696 |
| chr3 | 121323304 | 121323448 |
| chr3 | 121559821 | 121559945 |
| chr3 | 121670315 | 121670467 |
| chr3 | 121855659 | 121855801 |
| chr3 | 121921650 | 121921774 |
| chr3 | 122074662 | 122074788 |
| chr3 | 122177831 | 122177961 |
| chr3 | 122396974 | 122397108 |
| chr3 | 122479619 | 122479761 |
| chr3 | 122755693 | 122755863 |
| chr3 | 122810139 | 122810261 |
| chr3 | 122922355 | 122922499 |
| chr3 | 123127020 | 123127160 |
| chr3 | 123286017 | 123286139 |
| chr3 | 123399602 | 123399728 |
| chr3 | 123604714 | 123604836 |
| chr3 | 123804180 | 123804302 |
| chr3 | 123941030 | 123941172 |
| chr3 | 124094954 | 124095088 |
| chr3 | 124204890 | 124205016 |
| chr3 | 124273581 | 124273703 |
| chr3 | 124487299 | 124487423 |
| chr3 | 124652206 | 124652338 |
| chr3 | 124801709 | 124801851 |
| chr3 | 124862826 | 124862956 |
| chr3 | 125128607 | 125128765 |
| chr3 | 125287428 | 125287564 |
| chr3 | 125345230 | 125345362 |
| chr3 | 125509335 | 125509463 |
| chr3 | 125588727 | 125588940 |
| chr3 | 125604498 | 125604626 |
| chr3 | 125758166 | 125758290 |
| chr3 | 125868013 | 125868141 |
| chr3 | 125973533 | 125973669 |
| chr3 | 126128042 | 126128184 |
| chr3 | 126283015 | 126283157 |
| chr3 | 126363394 | 126363524 |
| chr3 | 126660404 | 126660536 |
| chr3 | 126667593 | 126667743 |
| chr3 | 126918350 | 126918480 |
| chr3 | 126965047 | 126965201 |
| chr3 | 127259170 | 127259298 |
| chr3 | 127280885 | 127281027 |
| chr3 | 127521294 | 127521434 |
| chr3 | 127576441 | 127576565 |
| chr3 | 127745637 | 127745763 |
| chr3 | 127934238 | 127934364 |
| chr3 | 128071719 | 128071855 |
| chr3 | 128301362 | 128301506 |
| chr3 | 128446360 | 128446482 |
| chr3 | 128528572 | 128528716 |
| chr3 | 128648061 | 128648193 |
| chr3 | 128785708 | 128785902 |
| chr3 | 128915639 | 128915904 |
| chr3 | 129139238 | 129139388 |
| chr3 | 129224041 | 129224163 |
| chr3 | 129390686 | 129390957 |
| chr3 | 129615966 | 129616094 |
| chr3 | 129663402 | 129663524 |
| chr3 | 129764465 | 129764585 |
| chr3 | 129771138 | 129771261 |
| chr3 | 129809373 | 129809493 |
| chr3 | 129855900 | 129856113 |
| chr3 | 129933566 | 129933708 |
| chr3 | 130016759 | 130016883 |
| chr3 | 130258595 | 130258731 |
| chr3 | 130303150 | 130303274 |
| chr3 | 130499212 | 130499350 |
| chr3 | 130661241 | 130661363 |
| chr3 | 130843952 | 130844094 |
| chr3 | 130951065 | 130951205 |
| chr3 | 131096713 | 131096855 |
| chr3 | 131169877 | 131169999 |
| chr3 | 131450616 | 131450738 |
| chr3 | 131607459 | 131607583 |
| chr3 | 131649917 | 131650063 |
| chr3 | 131843789 | 131843945 |
| chr3 | 131942162 | 131942286 |
| chr3 | 132120143 | 132120277 |
| chr3 | 132269534 | 132269658 |
| chr3 | 132458474 | 132458600 |
| chr3 | 132534397 | 132534541 |
| chr3 | 132725785 | 132725913 |
| chr3 | 132881033 | 132881157 |
| chr3 | 132987374 | 132987528 |
| chr3 | 133189116 | 133189244 |
| chr3 | 133295113 | 133295271 |
| chr3 | 133474746 | 133474916 |
| chr3 | 133674897 | 133675023 |
| chr3 | 133822490 | 133822646 |
| chr3 | 133919825 | 133919957 |
| chr3 | 134038834 | 134038958 |
| chr3 | 134206650 | 134206776 |
| chr3 | 134453918 | 134454060 |
| chr3 | 134553499 | 134553625 |
| chr3 | 134652715 | 134652843 |
| chr3 | 134778410 | 134778542 |
| chr3 | 134999852 | 134999976 |
| chr3 | 135079779 | 135079907 |
| chr3 | 135301202 | 135301324 |
| chr3 | 135459147 | 135459431 |
| chr3 | 135581169 | 135581325 |
| chr3 | 135755943 | 135756065 |
| chr3 | 135828762 | 135828980 |
| chr3 | 135976998 | 135977120 |
| chr3 | 136153420 | 136153556 |
| chr3 | 136410367 | 136410539 |
| chr3 | 136555710 | 136555834 |
| chr3 | 136695621 | 136695745 |
| chr3 | 136804932 | 136805072 |
| chr3 | 136870584 | 136870706 |
| chr3 | 137121864 | 137121994 |
| chr3 | 137214757 | 137214915 |
| chr3 | 137441078 | 137441204 |
| chr3 | 137467600 | 137467724 |
| chr3 | 137685870 | 137686008 |
| chr3 | 137856708 | 137856864 |
| chr3 | 137914415 | 137914683 |
| chr3 | 138107007 | 138107137 |
| chr3 | 138334853 | 138334989 |
| chr3 | 138468306 | 138468492 |
| chr3 | 138542973 | 138543094 |
| chr3 | 138663118 | 138663242 |
| chr3 | 138942955 | 138943077 |
| chr3 | 139101709 | 139101839 |
| chr3 | 139164786 | 139164922 |
| chr3 | 139353586 | 139353714 |
| chr3 | 139489583 | 139489723 |
| chr3 | 139581843 | 139581965 |
| chr3 | 139788484 | 139788610 |
| chr3 | 139930944 | 139931068 |
| chr3 | 140046526 | 140046652 |
| chr3 | 140254582 | 140254710 |
| chr3 | 140356575 | 140356717 |
| chr3 | 140542332 | 140542464 |
| chr3 | 140698378 | 140698516 |
| chr3 | 140778695 | 140778837 |
| chr3 | 141046785 | 141046911 |
| chr3 | 141101907 | 141102033 |
| chr3 | 141249346 | 141249468 |
| chr3 | 141457908 | 141458036 |
| chr3 | 141603807 | 141603939 |
| chr3 | 141767771 | 141767903 |
| chr3 | 141926701 | 141926851 |
| chr3 | 142015500 | 142015624 |
| chr3 | 142132667 | 142132791 |
| chr3 | 142327019 | 142327145 |
| chr3 | 142535331 | 142535467 |
| chr3 | 142609859 | 142609991 |
| chr3 | 142746936 | 142747066 |
| chr3 | 142953956 | 142954104 |
| chr3 | 143127007 | 143127145 |
| chr3 | 143163113 | 143163257 |
| chr3 | 143389939 | 143390083 |
| chr3 | 143596094 | 143596265 |
| chr3 | 143711232 | 143711370 |
| chr3 | 143778620 | 143778760 |
| chr3 | 143941883 | 143942007 |
| chr3 | 144148841 | 144148965 |
| chr3 | 144227925 | 144228047 |
| chr3 | 144491257 | 144491395 |
| chr3 | 144533341 | 144533465 |
| chr3 | 144778543 | 144778665 |
| chr3 | 144855077 | 144855213 |
| chr3 | 145086668 | 145086796 |
| chr3 | 145141723 | 145141847 |
| chr3 | 145357555 | 145357681 |
| chr3 | 145458874 | 145459004 |
| chr3 | 145697139 | 145697263 |
| chr3 | 145734963 | 145735117 |
| chr3 | 145888437 | 145888569 |
| chr3 | 146120946 | 146121068 |
| chr3 | 146302115 | 146302261 |
| chr3 | 146348494 | 146348618 |
| chr3 | 146551298 | 146551540 |
| chr3 | 146688436 | 146688566 |
| chr3 | 146809485 | 146809607 |
| chr3 | 146939240 | 146939378 |
| chr3 | 147148179 | 147148303 |
| chr3 | 147320530 | 147320660 |
| chr3 | 147417577 | 147417707 |
| chr3 | 147566519 | 147566643 |
| chr3 | 147696587 | 147696711 |
| chr3 | 147868888 | 147869014 |
| chr3 | 148102884 | 148103030 |
| chr3 | 148215075 | 148215205 |
| chr3 | 148369322 | 148369472 |
| chr3 | 148533323 | 148533447 |
| chr3 | 148661094 | 148661240 |
| chr3 | 148755789 | 148755949 |
| chr3 | 148987703 | 148987825 |
| chr3 | 149066910 | 149067032 |
| chr3 | 149284384 | 149284518 |
| chr3 | 149447851 | 149448009 |
| chr3 | 149532736 | 149532860 |
| chr3 | 149649538 | 149649659 |
| chr3 | 149870108 | 149870238 |
| chr3 | 150010844 | 150010982 |
| chr3 | 150092804 | 150092938 |
| chr3 | 150339021 | 150339153 |
| chr3 | 150382939 | 150383071 |
| chr3 | 150590104 | 150590250 |
| chr3 | 150714351 | 150714473 |
| chr3 | 150959193 | 150959323 |
| chr3 | 151087561 | 151087691 |
| chr3 | 151236508 | 151236642 |
| chr3 | 151403552 | 151403674 |
| chr3 | 151500416 | 151500550 |
| chr3 | 151579813 | 151579959 |
| chr3 | 151789979 | 151790121 |
| chr3 | 151930244 | 151930368 |
| chr3 | 152149747 | 152149883 |
| chr3 | 152276173 | 152276311 |
| chr3 | 152332870 | 152333008 |
| chr3 | 152552100 | 152552238 |
| chr3 | 152643857 | 152643997 |
| chr3 | 152825546 | 152825672 |
| chr3 | 153048825 | 153048949 |
| chr3 | 153160357 | 153160481 |
| chr3 | 153273762 | 153273894 |
| chr3 | 153444665 | 153444809 |
| chr3 | 153513300 | 153513436 |
| chr3 | 153774426 | 153774550 |
| chr3 | 153914846 | 153914974 |
| chr3 | 153999744 | 153999868 |
| chr3 | 154187720 | 154187848 |
| chr3 | 154326098 | 154326368 |
| chr3 | 154560639 | 154560791 |
| chr3 | 154693021 | 154693163 |
| chr3 | 154860834 | 154860956 |
| chr3 | 154920574 | 154920732 |
| chr3 | 155021725 | 155021855 |
| chr3 | 155181290 | 155181428 |
| chr3 | 155348880 | 155349004 |
| chr3 | 155490955 | 155491077 |
| chr3 | 155712162 | 155712310 |
| chr3 | 155872389 | 155872535 |
| chr3 | 155955411 | 155955533 |
| chr3 | 156127964 | 156128086 |
| chr3 | 156312344 | 156312488 |
| chr3 | 156427769 | 156427891 |
| chr3 | 156542549 | 156542703 |
| chr3 | 156711166 | 156711290 |
| chr3 | 156884623 | 156884755 |
| chr3 | 156968172 | 156968298 |
| chr3 | 157170958 | 157171088 |
| chr3 | 157371841 | 157371979 |
| chr3 | 157540756 | 157540894 |
| chr3 | 157586026 | 157586150 |
| chr3 | 157850158 | 157850292 |
| chr3 | 157892288 | 157892438 |
| chr3 | 158094340 | 158094480 |
| chr3 | 158206693 | 158206843 |
| chr3 | 158439343 | 158439483 |
| chr3 | 158578996 | 158579120 |
| chr3 | 158624734 | 158624886 |
| chr3 | 158833296 | 158833434 |
| chr3 | 158916796 | 158916940 |
| chr3 | 159105423 | 159105545 |
| chr3 | 159280404 | 159280544 |
| chr3 | 159414417 | 159414549 |
| chr3 | 159565361 | 159565495 |
| chr3 | 159678577 | 159678715 |
| chr3 | 159956485 | 159956607 |
| chr3 | 160065482 | 160065606 |
| chr3 | 160213050 | 160213172 |
| chr3 | 160382346 | 160382480 |
| chr3 | 160491632 | 160491776 |
| chr3 | 160619944 | 160620094 |
| chr3 | 160748935 | 160749075 |
| chr3 | 160936310 | 160936444 |
| chr3 | 161120447 | 161120571 |
| chr3 | 161283972 | 161284108 |
| chr3 | 161375512 | 161375668 |
| chr3 | 161547439 | 161547561 |
| chr3 | 161741208 | 161741330 |
| chr3 | 161832735 | 161832895 |
| chr3 | 161953045 | 161953183 |
| chr3 | 162079409 | 162079543 |
| chr3 | 162252903 | 162253047 |
| chr3 | 162496149 | 162496281 |
| chr3 | 162638868 | 162639018 |
| chr3 | 162690276 | 162690400 |
| chr3 | 162899793 | 162899939 |
| chr3 | 162971882 | 162972008 |
| chr3 | 163124858 | 163124988 |
| chr3 | 163288758 | 163288886 |
| chr3 | 163459230 | 163459366 |
| chr3 | 163643840 | 163643978 |
| chr3 | 163752534 | 163752664 |
| chr3 | 164003791 | 164003935 |
| chr3 | 164076049 | 164076179 |
| chr3 | 164183063 | 164183209 |
| chr3 | 164352775 | 164352909 |
| chr3 | 164603504 | 164603630 |
| chr3 | 164647402 | 164647538 |
| chr3 | 164899419 | 164899563 |
| chr3 | 165022146 | 165022282 |
| chr3 | 165142299 | 165142423 |
| chr3 | 165289336 | 165289476 |
| chr3 | 165449643 | 165449769 |
| chr3 | 165637493 | 165637617 |
| chr3 | 165733312 | 165733456 |
| chr3 | 165909381 | 165909505 |
| chr3 | 165989920 | 165990060 |
| chr3 | 166127343 | 166127471 |
| chr3 | 166311650 | 166311796 |
| chr3 | 166538254 | 166538394 |
| chr3 | 166650725 | 166650849 |
| chr3 | 166839123 | 166839245 |
| chr3 | 166886926 | 166887048 |
| chr3 | 167105676 | 167105800 |
| chr3 | 167282150 | 167282296 |
| chr3 | 167449331 | 167449453 |
| chr3 | 167535138 | 167535260 |
| chr3 | 167648128 | 167648250 |
| chr3 | 167803079 | 167803203 |
| chr3 | 167914652 | 167914792 |
| chr3 | 168093352 | 168093522 |
| chr3 | 168299141 | 168299265 |
| chr3 | 168444864 | 168445010 |
| chr3 | 168520759 | 168520881 |
| chr3 | 168687207 | 168687331 |
| chr3 | 168921525 | 168921659 |
| chr3 | 169057961 | 169058097 |
| chr3 | 169128198 | 169128326 |
| chr3 | 169390178 | 169390302 |
| chr3 | 169439443 | 169439569 |
| chr3 | 169666163 | 169666305 |
| chr3 | 169804249 | 169804399 |
| chr3 | 169887030 | 169887152 |
| chr3 | 170077631 | 170077795 |
| chr3 | 170293500 | 170293630 |
| chr3 | 170398984 | 170399106 |
| chr3 | 170467290 | 170467426 |
| chr3 | 170649675 | 170649799 |
| chr3 | 170796496 | 170796747 |
| chr3 | 171003716 | 171003870 |
| chr3 | 171161926 | 171162072 |
| chr3 | 171274889 | 171275017 |
| chr3 | 171364225 | 171364349 |
| chr3 | 171619464 | 171619586 |
| chr3 | 171754569 | 171754693 |
| chr3 | 171815921 | 171816045 |
| chr3 | 172040138 | 172040276 |
| chr3 | 172233281 | 172233407 |
| chr3 | 172370010 | 172370132 |
| chr3 | 172412964 | 172413088 |
| chr3 | 172648051 | 172648179 |
| chr3 | 172823077 | 172823199 |
| chr3 | 172986990 | 172987112 |
| chr3 | 173064616 | 173064738 |
| chr3 | 173231112 | 173231246 |
| chr3 | 173397530 | 173397662 |
| chr3 | 173590021 | 173590143 |
| chr3 | 173623091 | 173623221 |
| chr3 | 173895868 | 173896008 |
| chr3 | 173986349 | 173986485 |
| chr3 | 174105048 | 174105186 |
| chr3 | 174213126 | 174213258 |
| chr3 | 174414091 | 174414231 |
| chr3 | 174522852 | 174523000 |
| chr3 | 174715953 | 174716107 |
| chr3 | 174858258 | 174858406 |
| chr3 | 175024648 | 175024774 |
| chr3 | 175252303 | 175252443 |
| chr3 | 175343029 | 175343165 |
| chr3 | 175454621 | 175454753 |
| chr3 | 175599326 | 175599452 |
| chr3 | 175759696 | 175759834 |
| chr3 | 175993028 | 175993154 |
| chr3 | 176022555 | 176022691 |
| chr3 | 176298863 | 176298987 |
| chr3 | 176382744 | 176382874 |
| chr3 | 176496536 | 176496682 |
| chr3 | 176667205 | 176667339 |
| chr3 | 176880647 | 176880771 |
| chr3 | 177038819 | 177038967 |
| chr3 | 177082791 | 177082917 |
| chr3 | 177315997 | 177316133 |
| chr3 | 177495824 | 177495948 |
| chr3 | 177554148 | 177554286 |
| chr3 | 177798761 | 177798903 |
| chr3 | 177906993 | 177907135 |
| chr3 | 177976001 | 177976139 |
| chr3 | 178159374 | 178159512 |
| chr3 | 178399395 | 178399521 |
| chr3 | 178497145 | 178497271 |
| chr3 | 178631891 | 178632019 |
| chr3 | 178853987 | 178854127 |
| chr3 | 178969041 | 178969165 |
| chr3 | 179106793 | 179106923 |
| chr3 | 179246131 | 179246255 |
| chr3 | 179428420 | 179428544 |
| chr3 | 179549561 | 179549685 |
| chr3 | 179706366 | 179706498 |
| chr3 | 179786672 | 179786796 |
| chr3 | 180052840 | 180052974 |
| chr3 | 180174301 | 180174512 |
| chr3 | 180284373 | 180284511 |
| chr3 | 180438233 | 180438361 |
| chr3 | 180552099 | 180552237 |
| chr3 | 180700096 | 180700228 |
| chr3 | 180831931 | 180832059 |
| chr3 | 181001664 | 181001786 |
| chr3 | 181217972 | 181218094 |
| chr3 | 181272381 | 181272519 |
| chr3 | 181444002 | 181444125 |
| chr3 | 181592118 | 181592258 |
| chr3 | 181715493 | 181715625 |
| chr3 | 181983459 | 181983583 |
| chr3 | 182017449 | 182017575 |
| chr3 | 182265542 | 182265668 |
| chr3 | 182457309 | 182457453 |
| chr3 | 182521077 | 182521207 |
| chr3 | 182625607 | 182625767 |
| chr3 | 182826264 | 182826388 |
| chr3 | 183015664 | 183015790 |
| chr3 | 183113946 | 183114092 |
| chr3 | 183329840 | 183329966 |
| chr3 | 183500640 | 183500782 |
| chr3 | 183616006 | 183616156 |
| chr3 | 183691592 | 183691736 |
| chr3 | 183863735 | 183863863 |
| chr3 | 184092962 | 184093100 |
| chr3 | 184228881 | 184229027 |
| chr3 | 184398063 | 184398205 |
| chr3 | 184468656 | 184468780 |
| chr3 | 184617048 | 184617174 |
| chr3 | 184816249 | 184816375 |
| chr3 | 184932395 | 184932539 |
| chr3 | 185055388 | 185055512 |
| chr3 | 185175590 | 185175728 |
| chr3 | 185426703 | 185426855 |
| chr3 | 185567843 | 185567975 |
| chr3 | 185748980 | 185749126 |
| chr3 | 185798218 | 185798350 |
| chr3 | 186048595 | 186048727 |
| chr3 | 186161287 | 186161411 |
| chr3 | 186254758 | 186254884 |
| chr3 | 186497164 | 186497320 |
| chr3 | 186588174 | 186588304 |
| chr3 | 186690556 | 186690684 |
| chr3 | 186847907 | 186848029 |
| chr3 | 187012844 | 187012966 |
| chr3 | 187196365 | 187196505 |
| chr3 | 187311519 | 187311645 |
| chr3 | 187428820 | 187428942 |
| chr3 | 187568324 | 187568458 |
| chr3 | 187800096 | 187800218 |
| chr3 | 187874845 | 187874967 |
| chr3 | 188022231 | 188022361 |
| chr3 | 188178066 | 188178190 |
| chr3 | 188445548 | 188445694 |
| chr3 | 188600290 | 188600452 |
| chr3 | 188716844 | 188717133 |
| chr3 | 188869823 | 188869947 |
| chr3 | 189045226 | 189045356 |
| chr3 | 189080189 | 189080335 |
| chr3 | 189345992 | 189346400 |
| chr3 | 189441320 | 189441444 |
| chr3 | 189530123 | 189530269 |
| chr3 | 189762530 | 189762668 |
| chr3 | 189898114 | 189898274 |
| chr3 | 189982281 | 189982421 |
| chr3 | 190171290 | 190171414 |
| chr3 | 190358286 | 190358440 |
| chr3 | 190446445 | 190446573 |
| chr3 | 190620929 | 190621075 |
| chr3 | 190801657 | 19080181 |
| chr3 | 190861722 | 190861852 |
| chr3 | 191106265 | 191106391 |
| chr3 | 191213430 | 191213556 |
| chr3 | 191414715 | 191414863 |
| chr3 | 191470925 | 191471049 |
| chr3 | 191628387 | 191628511 |
| chr3 | 191893054 | 191893196 |
| chr3 | 192059773 | 192059905 |
| chr3 | 192094694 | 192094822 |
| chr3 | 192240993 | 192241119 |
| chr3 | 192389240 | 192389376 |
| chr3 | 192515477 | 192515625 |
| chr3 | 192706671 | 192706799 |
| chr3 | 192812795 | 192812951 |
| chr3 | 193033329 | 193033471 |
| chr3 | 193192715 | 193192851 |
| chr3 | 193408345 | 193408467 |
| chr3 | 193482581 | 193482727 |
| chr3 | 193568432 | 193568580 |
| chr3 | 193761057 | 193761207 |
| chr3 | 193963395 | 193963519 |
| chr3 | 194130737 | 194130861 |
| chr3 | 194286785 | 194286923 |
| chr3 | 194429639 | 194429777 |
| chr3 | 194470596 | 194470720 |
| chr3 | 194641258 | 194641396 |
| chr3 | 194776812 | 194776936 |
| chr3 | 195051859 | 195051981 |
| chr3 | 195104685 | 195104811 |
| chr3 | 195340591 | 195340712 |
| chr3 | 195465358 | 195465488 |
| chr3 | 195656433 | 195656652 |
| chr3 | 195735677 | 195735807 |
| chr3 | 195877527 | 195877653 |
| chr3 | 196107156 | 196107282 |
| chr3 | 196219720 | 196219842 |
| chr3 | 196308825 | 196308965 |
| chr3 | 196543604 | 196543726 |
| chr3 | 196687975 | 196688103 |
| chr3 | 196860118 | 196860240 |
| chr3 | 197008647 | 197008787 |
| chr3 | 197068421 | 197068571 |
| chr3 | 197195387 | 197195547 |
| chr3 | 197394939 | 197395112 |
| chr3 | 197495290 | 197495416 |
| chr3 | 197557599 | 197557723 |
| chr3 | 197607425 | 197607545 |
| chr3 | 197624390 | 197624518 |
| chr3 | 197633886 | 197634160 |
| chr3 | 197642324 | 197642456 |
| chr3 | 197646162 | 197646284 |
| chr3 | 197655805 | 197655941 |
| chr3 | 197665509 | 197665647 |
| chr3 | 197676167 | 197676291 |
| chr3 | 197686232 | 197686406 |
| chr3 | 197734259 | 197734426 |
| chr3 | 197839932 | 197840179 |
| chr3 | 197902863 | 197902993 |
| chr3 | 197907011 | 197907133 |
| chr3 | 197917697 | 197917827 |
| chr4 | 159007 | 159147 |
| chr4 | 242713 | 242835 |
| chr4 | 367841 | 368015 |
| chr4 | 429580 | 429701 |
| chr4 | 502847 | 502997 |
| chr4 | 540047 | 540171 |
| chr4 | 584643 | 584867 |
| chr4 | 639592 | 639730 |
| chr4 | 732829 | 732977 |
| chr4 | 928598 | 928722 |
| chr4 | 1058745 | 1058893 |
| chr4 | 1147287 | 1147417 |
| chr4 | 1386385 | 1386509 |
| chr4 | 1488311 | 1488501 |
| chr4 | 1647544 | 1647686 |
| chr4 | 1870373 | 1870494 |
| chr4 | 1878965 | 1879248 |
| chr4 | 2152393 | 2152537 |
| chr4 | 2310493 | 2310633 |
| chr4 | 2376540 | 2376662 |
| chr4 | 2505154 | 2505294 |
| chr4 | 2630957 | 2631087 |
| chr4 | 2861940 | 2862074 |
| chr4 | 2959695 | 2959851 |
| chr4 | 3104500 | 3104656 |
| chr4 | 3288182 | 3288306 |
| chr4 | 3398304 | 3398426 |
| chr4 | 3618104 | 3618282 |
| chr4 | 3698577 | 3698701 |
| chr4 | 3921132 | 3921256 |
| chr4 | 4001220 | 4001433 |
| chr4 | 4159620 | 4159792 |
| chr4 | 4300989 | 4301125 |
| chr4 | 4499680 | 4499806 |
| chr4 | 4689935 | 4690075 |
| chr4 | 4758238 | 4758376 |
| chr4 | 5013883 | 5014019 |
| chr4 | 5137326 | 5137450 |
| chr4 | 5229838 | 5229962 |
| chr4 | 5427845 | 5427993 |
| chr4 | 5575248 | 5575384 |
| chr4 | 5650846 | 5650970 |
| chr4 | 5852062 | 5852188 |
| chr4 | 5979399 | 5979525 |
| chr4 | 6103730 | 6103868 |
| chr4 | 6256859 | 6256985 |
| chr4 | 6480779 | 6480933 |
| chr4 | 6552094 | 6552224 |
| chr4 | 6701113 | 6701269 |
| chr4 | 6874746 | 6874900 |
| chr4 | 7009720 | 7009844 |
| chr4 | 7247293 | 7247417 |
| chr4 | 7368967 | 7369103 |
| chr4 | 7451133 | 7451275 |
| chr4 | 7591966 | 7592092 |
| chr4 | 7848086 | 7848210 |
| chr4 | 7977523 | 7977677 |
| chr4 | 8132879 | 8133003 |
| chr4 | 8314552 | 8314690 |
| chr4 | 8454880 | 8455022 |
| chr4 | 8565884 | 8566016 |
| chr4 | 8732434 | 8732555 |
| chr4 | 8831300 | 8831421 |
| chr4 | 8939368 | 8939489 |
| chr4 | 9025167 | 9025301 |
| chr4 | 9111103 | 9111239 |
| chr4 | 9375260 | 9375384 |
| chr4 | 9400465 | 9400585 |
| chr4 | 9559359 | 9559483 |
| chr4 | 9560741 | 9560963 |
| chr4 | 9646758 | 9646971 |
| chr4 | 9713364 | 9713488 |
| chr4 | 9739797 | 9739920 |
| chr4 | 9847824 | 9847948 |
| chr4 | 9990254 | 9990376 |
| chr4 | 10140925 | 10141051 |
| chr4 | 10400096 | 10400230 |
| chr4 | 10566976 | 10567100 |
| chr4 | 10660969 | 10661097 |
| chr4 | 10760134 | 10760284 |
| chr4 | 10910235 | 10910361 |
| chr4 | 11050040 | 11050164 |
| chr4 | 11243152 | 11243274 |
| chr4 | 11415312 | 11415438 |
| chr4 | 11556017 | 11556145 |
| chr4 | 11638385 | 11638523 |
| chr4 | 11868031 | 11868171 |
| chr4 | 11928992 | 11929142 |
| chr4 | 12215718 | 12215864 |
| chr4 | 12349024 | 12349152 |
| chr4 | 12517766 | 12517890 |
| chr4 | 12637234 | 12637386 |
| chr4 | 12713468 | 12713612 |
| chr4 | 12864018 | 12864144 |
| chr4 | 13106274 | 13106418 |
| chr4 | 13253518 | 13253650 |
| chr4 | 13324551 | 13324802 |
| chr4 | 13482969 | 13483111 |
| chr4 | 13721137 | 13721269 |
| chr4 | 13813292 | 13813416 |
| chr4 | 14001810 | 14001938 |
| chr4 | 14132430 | 14132556 |
| chr4 | 14256497 | 14256639 |
| chr4 | 14410772 | 14410900 |
| chr4 | 14503094 | 14503232 |
| chr4 | 14770684 | 14770816 |
| chr4 | 14823217 | 14823355 |
| chr4 | 15010552 | 15010678 |
| chr4 | 15127556 | 15127684 |
| chr4 | 15251933 | 15252057 |
| chr4 | 15506325 | 15506465 |
| chr4 | 15529701 | 15529825 |
| chr4 | 15723710 | 15723846 |
| chr4 | 15854649 | 15854791 |
| chr4 | 16079509 | 16079663 |
| chr4 | 16193800 | 16193956 |
| chr4 | 16427302 | 16427446 |
| chr4 | 16493608 | 16493754 |
| chr4 | 16598444 | 16598568 |
| chr4 | 16871225 | 16871363 |
| chr4 | 17022838 | 17022986 |
| chr4 | 17041800 | 17041924 |
| chr4 | 17207516 | 17207654 |
| chr4 | 17390712 | 17390850 |
| chr4 | 17523849 | 17523983 |
| chr4 | 17630138 | 17630280 |
| chr4 | 17797393 | 17797527 |
| chr4 | 17991371 | 17991503 |
| chr4 | 18169585 | 18169733 |
| chr4 | 18308627 | 18308767 |
| chr4 | 18409025 | 18409181 |
| chr4 | 18532326 | 18532466 |
| chr4 | 18783235 | 18783365 |
| chr4 | 18968555 | 18968709 |
| chr4 | 19094128 | 19094264 |
| chr4 | 19166320 | 19166444 |
| chr4 | 19352204 | 19352336 |
| chr4 | 19493525 | 19493647 |
| chr4 | 19578369 | 19578493 |
| chr4 | 19793895 | 19794019 |
| chr4 | 19960194 | 19960318 |
| chr4 | 20068350 | 20068472 |
| chr4 | 20264345 | 20264491 |
| chr4 | 20440388 | 20440510 |
| chr4 | 20539182 | 20539308 |
| chr4 | 20714704 | 20714850 |
| chr4 | 20782548 | 20782670 |
| chr4 | 20992022 | 20992146 |
| chr4 | 21169775 | 21169905 |
| chr4 | 21282378 | 21282518 |
| chr4 | 21381296 | 21381444 |
| chr4 | 21647880 | 21648008 |
| chr4 | 21687453 | 21687583 |
| chr4 | 21910060 | 21910202 |
| chr4 | 22100841 | 22100963 |
| chr4 | 22266260 | 22266396 |
| chr4 | 22370633 | 22370757 |
| chr4 | 22575003 | 22575129 |
| chr4 | 22647040 | 22647215 |
| chr4 | 22858431 | 22858565 |
| chr4 | 23006901 | 23007045 |
| chr4 | 23150550 | 23150674 |
| chr4 | 23238732 | 23238870 |
| chr4 | 23417497 | 23417623 |
| chr4 | 23532186 | 23532326 |
| chr4 | 23742849 | 23743003 |
| chr4 | 23859239 | 23859367 |
| chr4 | 24015094 | 24015226 |
| chr4 | 24162035 | 24162159 |
| chr4 | 24259966 | 24260098 |
| chr4 | 24462023 | 24462145 |
| chr4 | 24577147 | 24577279 |
| chr4 | 24716831 | 24716991 |
| chr4 | 24928303 | 24928459 |
| chr4 | 25069276 | 25069402 |
| chr4 | 25215030 | 25215170 |
| chr4 | 25349017 | 25349141 |
| chr4 | 25560425 | 25560549 |
| chr4 | 25687681 | 25687821 |
| chr4 | 25837232 | 25837370 |
| chr4 | 25883843 | 25883965 |
| chr4 | 26111515 | 26111641 |
| chr4 | 26186968 | 26187092 |
| chr4 | 26336153 | 26336277 |
| chr4 | 26577979 | 26578109 |
| chr4 | 26758066 | 26758192 |
| chr4 | 26824195 | 26824319 |
| chr4 | 27011273 | 27011411 |
| chr4 | 27099043 | 27099169 |
| chr4 | 27368105 | 27368229 |
| chr4 | 27475301 | 27475425 |
| chr4 | 27558789 | 27558925 |
| chr4 | 27776046 | 27776182 |
| chr4 | 27867840 | 27867998 |
| chr4 | 28029535 | 28029677 |
| chr4 | 28237745 | 28237867 |
| chr4 | 28397449 | 28397575 |
| chr4 | 28472329 | 28472451 |
| chr4 | 28604858 | 28604986 |
| chr4 | 28746038 | 28746162 |
| chr4 | 28965927 | 28966079 |
| chr4 | 29088169 | 29088301 |
| chr4 | 29245182 | 29245340 |
| chr4 | 29421750 | 29421888 |
| chr4 | 29551773 | 29551897 |
| chr4 | 29712471 | 29712595 |
| chr4 | 29826088 | 29826212 |
| chr4 | 30060870 | 30060998 |
| chr4 | 30174899 | 30175023 |
| chr4 | 30312982 | 30313106 |
| chr4 | 30437056 | 30437184 |
| chr4 | 30614264 | 30614392 |
| chr4 | 30744244 | 30744368 |
| chr4 | 30860547 | 30860671 |
| chr4 | 31024163 | 31024289 |
| chr4 | 31235788 | 31235912 |
| chr4 | 31312977 | 31313111 |
| chr4 | 31518200 | 31518322 |
| chr4 | 31678127 | 31678269 |
| chr4 | 31808624 | 31808754 |
| chr4 | 31904342 | 31904468 |
| chr4 | 32129946 | 32130092 |
| chr4 | 32287794 | 32287936 |
| chr4 | 32356913 | 32357047 |
| chr4 | 32561961 | 32562093 |
| chr4 | 32768989 | 32769111 |
| chr4 | 32896563 | 32896691 |
| chr4 | 33024992 | 33025118 |
| chr4 | 33146269 | 33146401 |
| chr4 | 33327079 | 33327221 |
| chr4 | 33507126 | 33507294 |
| chr4 | 33677040 | 33677168 |
| chr4 | 33814760 | 33814886 |
| chr4 | 33920033 | 33920161 |
| chr4 | 34122791 | 34122921 |
| chr4 | 34248146 | 34248272 |
| chr4 | 34294820 | 34294942 |
| chr4 | 34550915 | 34551057 |
| chr4 | 34700549 | 34700681 |
| chr4 | 34855380 | 34855504 |
| chr4 | 34949905 | 34950027 |
| chr4 | 35127737 | 35127881 |
| chr4 | 35191074 | 35191198 |
| chr4 | 35426924 | 35427082 |
| chr4 | 35513533 | 35513685 |
| chr4 | 35688933 | 35689099 |
| chr4 | 35857870 | 35858012 |
| chr4 | 36052423 | 36052567 |
| chr4 | 36089468 | 36089610 |
| chr4 | 36229099 | 36229241 |
| chr4 | 36412923 | 36413065 |
| chr4 | 36555105 | 36555243 |
| chr4 | 36775953 | 36776081 |
| chr4 | 36853303 | 36853443 |
| chr4 | 37021617 | 37021751 |
| chr4 | 37238330 | 37238474 |
| chr4 | 37291298 | 37291424 |
| chr4 | 37428184 | 37428318 |
| chr4 | 37703989 | 37704117 |
| chr4 | 37804763 | 37804907 |
| chr4 | 37924309 | 37924435 |
| chr4 | 38162485 | 38162653 |
| chr4 | 38216159 | 38216297 |
| chr4 | 38433013 | 38433153 |
| chr4 | 38488465 | 38488587 |
| chr4 | 38664234 | 38664360 |
| chr4 | 38792472 | 38792598 |
| chr4 | 38954220 | 38954344 |
| chr4 | 39084087 | 39084229 |
| chr4 | 39269264 | 39269400 |
| chr4 | 39500160 | 39500284 |
| chr4 | 39657228 | 39657368 |
| chr4 | 39822723 | 39822855 |
| chr4 | 39884512 | 39884664 |
| chr4 | 40097517 | 40097639 |
| chr4 | 40128104 | 40128228 |
| chr4 | 40291406 | 40291534 |
| chr4 | 40502887 | 40503027 |
| chr4 | 40638357 | 40638481 |
| chr4 | 40797605 | 40797781 |
| chr4 | 40946134 | 40946260 |
| chr4 | 41049139 | 41049277 |
| chr4 | 41200456 | 41200610 |
| chr4 | 41435921 | 41436045 |
| chr4 | 41565845 | 41565975 |
| chr4 | 41757526 | 41757670 |
| chr4 | 41808973 | 41809101 |
| chr4 | 42008716 | 42008974 |
| chr4 | 42105791 | 42105913 |
| chr4 | 42376268 | 42376396 |
| chr4 | 42475306 | 42475436 |
| chr4 | 42568249 | 42568373 |
| chr4 | 42694101 | 42694229 |
| chr4 | 42889104 | 42889242 |
| chr4 | 43066174 | 43066308 |
| chr4 | 43246561 | 43246707 |
| chr4 | 43317184 | 43317318 |
| chr4 | 43484240 | 43484376 |
| chr4 | 43601079 | 43601213 |
| chr4 | 43786403 | 43786527 |
| chr4 | 43981288 | 43981436 |
| chr4 | 44034342 | 44034468 |
| chr4 | 44257037 | 44257193 |
| chr4 | 44460980 | 44461110 |
| chr4 | 44610074 | 44610216 |
| chr4 | 44773486 | 44773610 |
| chr4 | 44808064 | 44808206 |
| chr4 | 45008845 | 45008979 |
| chr4 | 45093215 | 45093363 |
| chr4 | 45232250 | 45232384 |
| chr4 | 45522539 | 45522663 |
| chr4 | 45640679 | 45640813 |
| chr4 | 45720332 | 45720456 |
| chr4 | 45937481 | 45937625 |
| chr4 | 46093271 | 46093401 |
| chr4 | 46164528 | 46164674 |
| chr4 | 46404325 | 46404457 |
| chr4 | 46541647 | 46541783 |
| chr4 | 46683154 | 46683278 |
| chr4 | 46845952 | 46846090 |
| chr4 | 46944154 | 46944300 |
| chr4 | 47085255 | 47085377 |
| chr4 | 47241996 | 47242136 |
| chr4 | 47467442 | 47467564 |
| chr4 | 47526399 | 47526521 |
| chr4 | 47722695 | 47722831 |
| chr4 | 47817401 | 47817539 |
| chr4 | 47982818 | 47982960 |
| chr4 | 48148752 | 48148900 |
| chr4 | 48361425 | 48361555 |
| chr4 | 48495539 | 48495681 |
| chr4 | 48598319 | 48598445 |
| chr4 | 48777249 | 48777391 |
| chr4 | 48956229 | 48956355 |
| chr4 | 49051675 | 49051829 |
| chr4 | 49092600 | 49092726 |
| chr4 | 49186876 | 49186996 |
| chr4 | 49613915 | 49614035 |
| chr4 | 52705631 | 52705757 |
| chr4 | 52809669 | 52809793 |
| chr4 | 52993719 | 52993847 |
| chr4 | 53136214 | 53136354 |
| chr4 | 53266669 | 53266817 |
| chr4 | 53397533 | 53397657 |
| chr4 | 53571823 | 53571950 |
| chr4 | 53643419 | 53643561 |
| chr4 | 53881635 | 53881761 |
| chr4 | 53947176 | 53947308 |
| chr4 | 54180464 | 54180606 |
| chr4 | 54235317 | 54235449 |
| chr4 | 54454494 | 54454652 |
| chr4 | 54575350 | 54575484 |
| chr4 | 54730838 | 54730968 |
| chr4 | 54851023 | 54851165 |
| chr4 | 55070270 | 55070402 |
| chr4 | 55173843 | 55173967 |
| chr4 | 55286950 | 55287110 |
| chr4 | 55449564 | 55449692 |
| chr4 | 55590581 | 55590705 |
| chr4 | 55746141 | 55746291 |
| chr4 | 55983151 | 55983293 |
| chr4 | 56038638 | 56038768 |
| chr4 | 56238757 | 56238889 |
| chr4 | 56363841 | 56363963 |
| chr4 | 56530896 | 56531018 |
| chr4 | 56702905 | 56703047 |
| chr4 | 56864413 | 56864535 |
| chr4 | 56997312 | 56997438 |
| chr4 | 57188476 | 57188626 |
| chr4 | 57357166 | 57357298 |
| chr4 | 57470203 | 57470331 |
| chr4 | 57653880 | 57654004 |
| chr4 | 57810239 | 57810371 |
| chr4 | 57897788 | 57897922 |
| chr4 | 58085160 | 58085312 |
| chr4 | 58150628 | 58150766 |
| chr4 | 58417809 | 58417930 |
| chr4 | 58476971 | 58477107 |
| chr4 | 58589333 | 58589457 |
| chr4 | 58865521 | 58865651 |
| chr4 | 58968361 | 58968493 |
| chr4 | 59155355 | 59155479 |
| chr4 | 59279669 | 59279791 |
| chr4 | 59411601 | 59411753 |
| chr4 | 59503652 | 59503774 |
| chr4 | 59720379 | 59720507 |
| chr4 | 59800401 | 59800559 |
| chr4 | 59928983 | 59929117 |
| chr4 | 60134476 | 60134608 |
| chr4 | 60278714 | 60278854 |
| chr4 | 60458760 | 60458908 |
| chr4 | 60675969 | 60676091 |
| chr4 | 60803072 | 60803196 |
| chr4 | 60925955 | 60926083 |
| chr4 | 61119438 | 61119578 |
| chr4 | 61238549 | 61238695 |
| chr4 | 61296608 | 61296748 |
| chr4 | 61528827 | 61528959 |
| chr4 | 61682409 | 61682559 |
| chr4 | 61836106 | 61836248 |
| chr4 | 62013788 | 62013914 |
| chr4 | 62087028 | 62087170 |
| chr4 | 62261777 | 62261903 |
| chr4 | 62440791 | 62440915 |
| chr4 | 62580523 | 62580645 |
| chr4 | 62744457 | 62744587 |
| chr4 | 62872715 | 62872841 |
| chr4 | 62988246 | 62988384 |
| chr4 | 63158881 | 63159002 |
| chr4 | 63236754 | 63236880 |
| chr4 | 63400989 | 63401133 |
| chr4 | 63546126 | 63546250 |
| chr4 | 63747818 | 63747956 |
| chr4 | 63943973 | 63944119 |
| chr4 | 64117265 | 64117389 |
| chr4 | 64166621 | 64166759 |
| chr4 | 64358050 | 64358190 |
| chr4 | 64526480 | 64526606 |
| chr4 | 64661879 | 64662019 |
| chr4 | 64763345 | 64763499 |
| chr4 | 64900949 | 64901071 |
| chr4 | 65164833 | 65164957 |
| chr4 | 65257676 | 65257804 |
| chr4 | 65392355 | 65392489 |
| chr4 | 65574830 | 65574962 |
| chr4 | 65643028 | 65643152 |
| chr4 | 65843786 | 65843926 |
| chr4 | 65995996 | 65996118 |
| chr4 | 66188620 | 66188760 |
| chr4 | 66272125 | 66272253 |
| chr4 | 66384259 | 66384391 |
| chr4 | 66550672 | 66550806 |
| chr4 | 66700791 | 66700939 |
| chr4 | 66941340 | 66941462 |
| chr4 | 67050613 | 67050761 |
| chr4 | 67201284 | 67201408 |
| chr4 | 67282634 | 67282778 |
| chr4 | 67523450 | 67523590 |
| chr4 | 67721263 | 67721385 |
| chr4 | 67784340 | 67784462 |
| chr4 | 67904553 | 67904683 |
| chr4 | 68126712 | 68126850 |
| chr4 | 68297419 | 68297547 |
| chr4 | 68428675 | 68428803 |
| chr4 | 68563293 | 68563435 |
| chr4 | 68635020 | 68635160 |
| chr4 | 68913096 | 68913238 |
| chr4 | 68989088 | 68989214 |
| chr4 | 69109113 | 69109265 |
| chr4 | 69321901 | 69322059 |
| chr4 | 69527006 | 69527130 |
| chr4 | 69606857 | 69606997 |
| chr4 | 69705094 | 69705359 |
| chr4 | 69853773 | 69853901 |
| chr4 | 70025627 | 70025750 |
| chr4 | 70198070 | 70198202 |
| chr4 | 70406499 | 70406625 |
| chr4 | 70477518 | 70477642 |
| chr4 | 70708417 | 70708545 |
| chr4 | 70789118 | 70789240 |
| chr4 | 70911678 | 70911808 |
| chr4 | 71127192 | 71127320 |
| chr4 | 71220987 | 71221137 |
| chr4 | 71442370 | 71442492 |
| chr4 | 71619529 | 71619659 |
| chr4 | 71761690 | 71761812 |
| chr4 | 71893774 | 71893916 |
| chr4 | 71959278 | 71959422 |
| chr4 | 72117619 | 72117745 |
| chr4 | 72312576 | 72312702 |
| chr4 | 72514149 | 72514271 |
| chr4 | 72563361 | 72563485 |
| chr4 | 72738627 | 72738753 |
| chr4 | 72869599 | 72869729 |
| chr4 | 73011870 | 73012000 |
| chr4 | 73130845 | 73130995 |
| chr4 | 73303124 | 73303268 |
| chr4 | 73572373 | 73572497 |
| chr4 | 73613961 | 73614107 |
| chr4 | 73809868 | 73809992 |
| chr4 | 73896291 | 73896429 |
| chr4 | 74141370 | 74141504 |
| chr4 | 74236190 | 74236328 |
| chr4 | 74351934 | 74352082 |
| chr4 | 74543133 | 74543254 |
| chr4 | 74715229 | 74715369 |
| chr4 | 74814879 | 74815000 |
| chr4 | 75014601 | 75014858 |
| chr4 | 75223000 | 75223128 |
| chr4 | 75263707 | 75263831 |
| chr4 | 75387917 | 75388051 |
| chr4 | 75584145 | 75584267 |
| chr4 | 75720833 | 75721001 |
| chr4 | 75867207 | 75867331 |
| chr4 | 76072646 | 76072786 |
| chr4 | 76193721 | 76193843 |
| chr4 | 76282962 | 76283114 |
| chr4 | 76438351 | 76438477 |
| chr4 | 76633202 | 76633389 |
| chr4 | 76778502 | 76778626 |
| chr4 | 76976971 | 76977099 |
| chr4 | 77077395 | 77077527 |
| chr4 | 77286805 | 77286931 |
| chr4 | 77433848 | 77433990 |
| chr4 | 77490934 | 77491058 |
| chr4 | 77724703 | 77724835 |
| chr4 | 77906981 | 77907115 |
| chr4 | 78012767 | 78012909 |
| chr4 | 78191686 | 78191848 |
| chr4 | 78240073 | 78240223 |
| chr4 | 78510844 | 78510982 |
| chr4 | 78605331 | 78605457 |
| chr4 | 78743327 | 78743451 |
| chr4 | 78944245 | 78944377 |
| chr4 | 79033374 | 79033516 |
| chr4 | 79256191 | 79256313 |
| chr4 | 79368738 | 79368874 |
| chr4 | 79510378 | 79510506 |
| chr4 | 79592227 | 79592361 |
| chr4 | 79838726 | 79838848 |
| chr4 | 79936516 | 79936656 |
| chr4 | 80070337 | 80070594 |
| chr4 | 80196479 | 80196655 |
| chr4 | 80394550 | 80394678 |
| chr4 | 80565601 | 80565741 |
| chr4 | 80744461 | 80744589 |
| chr4 | 80887498 | 80887644 |
| chr4 | 80972252 | 80972398 |
| chr4 | 81152159 | 81152299 |
| chr4 | 81281975 | 81282228 |
| chr4 | 81505921 | 81506045 |
| chr4 | 81667635 | 81667779 |
| chr4 | 81773860 | 81774010 |
| chr4 | 81905960 | 81906098 |
| chr4 | 82014859 | 82014983 |
| chr4 | 82177265 | 82177387 |
| chr4 | 82319884 | 82320040 |
| chr4 | 82433305 | 82433433 |
| chr4 | 82720285 | 82720415 |
| chr4 | 82766093 | 82766217 |
| chr4 | 82900071 | 82900211 |
| chr4 | 83171257 | 83171391 |
| chr4 | 83300713 | 83300839 |
| chr4 | 83431722 | 83431848 |
| chr4 | 83509407 | 83509533 |
| chr4 | 83758912 | 83759052 |
| chr4 | 83800745 | 83800887 |
| chr4 | 83961527 | 83961677 |
| chr4 | 84172759 | 84172909 |
| chr4 | 84287719 | 84287861 |
| chr4 | 84411171 | 84411303 |
| chr4 | 84647254 | 84647392 |
| chr4 | 84717577 | 84717701 |
| chr4 | 84881882 | 84882004 |
| chr4 | 85058998 | 85059130 |
| chr4 | 85252594 | 85252738 |
| chr4 | 85407694 | 85407848 |
| chr4 | 85449673 | 85449811 |
| chr4 | 85667753 | 85667875 |
| chr4 | 85729852 | 85730000 |
| chr4 | 85959262 | 85959386 |
| chr4 | 86062071 | 86062195 |
| chr4 | 86270269 | 86270393 |
| chr4 | 86384239 | 86384417 |
| chr4 | 86528126 | 86528254 |
| chr4 | 86648699 | 86648831 |
| chr4 | 86927109 | 86927233 |
| chr4 | 87021848 | 87021972 |
| chr4 | 87082482 | 87082677 |
| chr4 | 87227949 | 87228095 |
| chr4 | 87494463 | 87494589 |
| chr4 | 87609098 | 87609354 |
| chr4 | 87742390 | 87742524 |
| chr4 | 87852018 | 87852142 |
| chr4 | 88000349 | 88000471 |
| chr4 | 88211018 | 88211142 |
| chr4 | 88349751 | 88349873 |
| chr4 | 88451527 | 88451659 |
| chr4 | 88707087 | 88707211 |
| chr4 | 88728868 | 88728992 |
| chr4 | 88952024 | 88952146 |
| chr4 | 89050934 | 89051074 |
| chr4 | 89312466 | 89312608 |
| chr4 | 89355013 | 89355159 |
| chr4 | 89599765 | 89599897 |
| chr4 | 89777001 | 89777129 |
| chr4 | 89860777 | 89860929 |
| chr4 | 89935306 | 89935438 |
| chr4 | 90094199 | 90094321 |
| chr4 | 90329640 | 90329902 |
| chr4 | 90454198 | 90454332 |
| chr4 | 90636922 | 90637064 |
| chr4 | 90739479 | 90739623 |
| chr4 | 90836483 | 90836607 |
| chr4 | 91008817 | 91008957 |
| chr4 | 91194768 | 91194892 |
| chr4 | 91427141 | 91427267 |
| chr4 | 91556098 | 91556234 |
| chr4 | 91689673 | 91689805 |
| chr4 | 91796257 | 91796391 |
| chr4 | 91952229 | 91952361 |
| chr4 | 92112023 | 92112167 |
| chr4 | 92285021 | 92285147 |
| chr4 | 92429407 | 92429533 |
| chr4 | 92602939 | 92603067 |
| chr4 | 92638698 | 92638838 |
| chr4 | 92808235 | 92808361 |
| chr4 | 92943430 | 92943554 |
| chr4 | 93154764 | 93154946 |
| chr4 | 93236330 | 93236470 |
| chr4 | 93466852 | 93466992 |
| chr4 | 93527982 | 93528128 |
| chr4 | 93678575 | 93678711 |
| chr4 | 93974127 | 93974249 |
| chr4 | 93987187 | 93987333 |
| chr4 | 94175522 | 94175666 |
| chr4 | 94426159 | 94426295 |
| chr4 | 94574867 | 94575013 |
| chr4 | 94684832 | 94684960 |
| chr4 | 94793257 | 94793407 |
| chr4 | 94964571 | 94964701 |
| chr4 | 95060906 | 95061032 |
| chr4 | 95268714 | 95268836 |
| chr4 | 95385452 | 95385596 |
| chr4 | 95497580 | 95497706 |
| chr4 | 95672355 | 95672485 |
| chr4 | 95844113 | 95844241 |
| chr4 | 96044503 | 96044625 |
| chr4 | 96175376 | 96175512 |
| chr4 | 96279588 | 96279712 |
| chr4 | 96518474 | 96518598 |
| chr4 | 96608480 | 96608608 |
| chr4 | 96783212 | 96783350 |
| chr4 | 96900790 | 96900912 |
| chr4 | 97124201 | 97124329 |
| chr4 | 97233619 | 97233751 |
| chr4 | 97315429 | 97315555 |
| chr4 | 97448945 | 97449081 |
| chr4 | 97602475 | 97602611 |
| chr4 | 97782515 | 97782643 |
| chr4 | 97918388 | 97918510 |
| chr4 | 98144804 | 98144950 |
| chr4 | 98311462 | 98311598 |
| chr4 | 98437049 | 98437193 |
| chr4 | 98511969 | 98512117 |
| chr4 | 98772147 | 98772271 |
| chr4 | 98897020 | 98897150 |
| chr4 | 98931584 | 98931708 |
| chr4 | 99127774 | 99128037 |
| chr4 | 99259486 | 99259763 |
| chr4 | 99425315 | 99425441 |
| chr4 | 99561999 | 99562131 |
| chr4 | 99811880 | 99812024 |
| chr4 | 99970763 | 99970903 |
| chr4 | 100060762 | 100060890 |
| chr4 | 100186922 | 100187048 |
| chr4 | 100354167 | 100354317 |
| chr4 | 100459617 | 100459745 |
| chr4 | 100643402 | 100643526 |
| chr4 | 100799905 | 100800047 |
| chr4 | 101006649 | 101006793 |
| chr4 | 101074985 | 101075113 |
| chr4 | 101238491 | 101238643 |
| chr4 | 101454125 | 101454257 |
| chr4 | 101509980 | 101510102 |
| chr4 | 101660936 | 101661066 |
| chr4 | 101923982 | 101924108 |
| chr4 | 101986673 | 101986795 |
| chr4 | 102132433 | 102132583 |
| chr4 | 102355853 | 102356007 |
| chr4 | 102432944 | 102433072 |
| chr4 | 102588427 | 102588557 |
| chr4 | 102769609 | 102769755 |
| chr4 | 102963823 | 102963945 |
| chr4 | 103121030 | 103121154 |
| chr4 | 103190410 | 103190534 |
| chr4 | 103283242 | 103283390 |
| chr4 | 103481277 | 103481399 |
| chr4 | 103617981 | 103618111 |
| chr4 | 103797282 | 103797410 |
| chr4 | 103977886 | 103978022 |
| chr4 | 104065965 | 104066103 |
| chr4 | 104261181 | 104261305 |
| chr4 | 104362714 | 104362836 |
| chr4 | 104619036 | 104619160 |
| chr4 | 104678311 | 104678447 |
| chr4 | 104860323 | 104860449 |
| chr4 | 105068527 | 105068651 |
| chr4 | 105110150 | 105110288 |
| chr4 | 105291523 | 105291661 |
| chr4 | 105480813 | 105480943 |
| chr4 | 105541337 | 105541473 |
| chr4 | 105716464 | 105716588 |
| chr4 | 105890459 | 105890587 |
| chr4 | 106010351 | 106010487 |
| chr4 | 106140457 | 106140579 |
| chr4 | 106418848 | 106418998 |
| chr4 | 106552645 | 106552767 |
| chr4 | 106687245 | 106687375 |
| chr4 | 106850426 | 106850558 |
| chr4 | 106966655 | 106966787 |
| chr4 | 107095435 | 107095561 |
| chr4 | 107231004 | 107231130 |
| chr4 | 107332544 | 107332696 |
| chr4 | 107618997 | 107619240 |
| chr4 | 107763804 | 107763988 |
| chr4 | 107887291 | 107887427 |
| chr4 | 108051422 | 108051566 |
| chr4 | 108158063 | 108158189 |
| chr4 | 108355964 | 108356092 |
| chr4 | 108413610 | 108413738 |
| chr4 | 108544853 | 108545021 |
| chr4 | 108779257 | 108779403 |
| chr4 | 108918330 | 108918608 |
| chr4 | 109104592 | 109104730 |
| chr4 | 109231048 | 109231174 |
| chr4 | 109374627 | 109374751 |
| chr4 | 109458654 | 109458808 |
| chr4 | 109704943 | 109705071 |
| chr4 | 109735437 | 109735561 |
| chr4 | 109951745 | 109951879 |
| chr4 | 110132790 | 110132930 |
| chr4 | 110216827 | 110216965 |
| chr4 | 110377783 | 110377909 |
| chr4 | 110576787 | 110576911 |
| chr4 | 110694881 | 110695043 |
| chr4 | 110798491 | 110798619 |
| chr4 | 111072911 | 111073035 |
| chr4 | 111127125 | 111127261 |
| chr4 | 111240428 | 111240570 |
| chr4 | 111498591 | 111498713 |
| chr4 | 111615652 | 111615780 |
| chr4 | 111706221 | 111706359 |
| chr4 | 111962442 | 111962580 |
| chr4 | 112003552 | 112003674 |
| chr4 | 112184632 | 112184778 |
| chr4 | 112307301 | 112307433 |
| chr4 | 112551398 | 112551526 |
| chr4 | 112617587 | 112617733 |
| chr4 | 112842565 | 112842687 |
| chr4 | 112975687 | 112975809 |
| chr4 | 113079321 | 113079473 |
| chr4 | 113323568 | 113323708 |
| chr4 | 113402427 | 113402575 |
| chr4 | 113599152 | 113599310 |
| chr4 | 113718842 | 113718970 |
| chr4 | 113905988 | 113906126 |
| chr4 | 114068871 | 114069015 |
| chr4 | 114124275 | 114124429 |
| chr4 | 114356111 | 114356233 |
| chr4 | 114478420 | 114478568 |
| chr4 | 114667961 | 114668107 |
| chr4 | 114742309 | 114742433 |
| chr4 | 114943657 | 114943783 |
| chr4 | 115013036 | 115013162 |
| chr4 | 115228428 | 115228568 |
| chr4 | 115330537 | 115330681 |
| chr4 | 115497576 | 115497710 |
| chr4 | 115586225 | 115586367 |
| chr4 | 115763507 | 115763629 |
| chr4 | 115924461 | 115924587 |
| chr4 | 116059279 | 116059403 |
| chr4 | 116186797 | 116186923 |
| chr4 | 116436768 | 116436920 |
| chr4 | 116612088 | 116612212 |
| chr4 | 116702112 | 116702238 |
| chr4 | 116912915 | 116913055 |
| chr4 | 116962098 | 116962220 |
| chr4 | 117097469 | 117097611 |
| chr4 | 117298112 | 117298236 |
| chr4 | 117426541 | 117426673 |
| chr4 | 117650608 | 117650742 |
| chr4 | 117735304 | 117735434 |
| chr4 | 117857278 | 117857410 |
| chr4 | 118087453 | 118087587 |
| chr4 | 118254666 | 118254788 |
| chr4 | 118280785 | 118280909 |
| chr4 | 118450653 | 118450781 |
| chr4 | 118682714 | 118682868 |
| chr4 | 118736837 | 118736981 |
| chr4 | 118879461 | 118879611 |
| chr4 | 119093222 | 119093346 |
| chr4 | 119272904 | 119273046 |
| chr4 | 119331309 | 119331433 |
| chr4 | 119516453 | 119516575 |
| chr4 | 119521591 | 119521721 |
| chr4 | 119592891 | 119593017 |
| chr4 | 119771234 | 119771356 |
| chr4 | 119915766 | 119915908 |
| chr4 | 120063679 | 120063803 |
| chr4 | 120161490 | 120161630 |
| chr4 | 120250807 | 120250928 |
| chr4 | 120361116 | 120361246 |
| chr4 | 120371795 | 120371915 |
| chr4 | 120411833 | 120411955 |
| chr4 | 120648994 | 120649120 |
| chr4 | 120738963 | 120739095 |
| chr4 | 120847367 | 120847499 |
| chr4 | 120983874 | 120984004 |
| chr4 | 121261720 | 121261844 |
| chr4 | 121298168 | 121298296 |
| chr4 | 121464653 | 121464779 |
| chr4 | 121588519 | 121588655 |
| chr4 | 121875255 | 121875381 |
| chr4 | 121935252 | 121935402 |
| chr4 | 122143620 | 122143742 |
| chr4 | 122244509 | 122244659 |
| chr4 | 122359290 | 122359440 |
| chr4 | 122547298 | 122547442 |
| chr4 | 122721346 | 122721484 |
| chr4 | 122896414 | 122896538 |
| chr4 | 122932102 | 122932230 |
| chr4 | 123126392 | 123126538 |
| chr4 | 123316024 | 123316146 |
| chr4 | 123456369 | 123456515 |
| chr4 | 123578806 | 123578938 |
| chr4 | 123696849 | 123696989 |
| chr4 | 123931840 | 123931966 |
| chr4 | 124087735 | 124087857 |
| chr4 | 124233026 | 124233148 |
| chr4 | 124320882 | 124321012 |
| chr4 | 124512745 | 124512871 |
| chr4 | 124667781 | 124667905 |
| chr4 | 124740958 | 124741094 |
| chr4 | 124920068 | 124920212 |
| chr4 | 125082393 | 125082517 |
| chr4 | 125254598 | 125254730 |
| chr4 | 125469188 | 125469312 |
| chr4 | 125518203 | 125518337 |
| chr4 | 125725306 | 125725430 |
| chr4 | 125805168 | 125805310 |
| chr4 | 125927445 | 125927575 |
| chr4 | 126188765 | 126188891 |
| chr4 | 126319427 | 126319581 |
| chr4 | 126490263 | 126490387 |
| chr4 | 126672093 | 126672219 |
| chr4 | 126823490 | 126823612 |
| chr4 | 126958676 | 126958810 |
| chr4 | 127080305 | 127080431 |
| chr4 | 127235663 | 127235795 |
| chr4 | 127336011 | 127336135 |
| chr4 | 127458898 | 127459026 |
| chr4 | 127677695 | 127677823 |
| chr4 | 127831205 | 127831349 |
| chr4 | 128024712 | 128024834 |
| chr4 | 128167015 | 128167143 |
| chr4 | 128202846 | 128202968 |
| chr4 | 128442311 | 128442455 |
| chr4 | 128624708 | 128624844 |
| chr4 | 128655746 | 128655904 |
| chr4 | 128856221 | 128856437 |
| chr4 | 128986798 | 128986926 |
| chr4 | 129190068 | 129190190 |
| chr4 | 129311771 | 129311907 |
| chr4 | 129381388 | 129381510 |
| chr4 | 129557969 | 129558109 |
| chr4 | 129759661 | 129759795 |
| chr4 | 129919657 | 129919787 |
| chr4 | 130077068 | 130077192 |
| chr4 | 130171907 | 130172029 |
| chr4 | 130380122 | 130380248 |
| chr4 | 130448712 | 130448834 |
| chr4 | 130627136 | 130627258 |
| chr4 | 130758391 | 130758529 |
| chr4 | 130975008 | 130975146 |
| chr4 | 131049425 | 131049571 |
| chr4 | 131183750 | 131183872 |
| chr4 | 131422739 | 131422865 |
| chr4 | 131587226 | 131587372 |
| chr4 | 131733646 | 131733916 |
| chr4 | 131834329 | 131834455 |
| chr4 | 132011013 | 132011145 |
| chr4 | 132148999 | 132149123 |
| chr4 | 132296391 | 132296523 |
| chr4 | 132409517 | 132409663 |
| chr4 | 132624929 | 132625067 |
| chr4 | 132630988 | 132631110 |
| chr4 | 132696611 | 132696741 |
| chr4 | 132794092 | 132794214 |
| chr4 | 132822001 | 132822141 |
| chr4 | 132964589 | 132964721 |
| chr4 | 133003465 | 133003591 |
| chr4 | 133162360 | 133162500 |
| chr4 | 133318427 | 133318573 |
| chr4 | 133432223 | 133432367 |
| chr4 | 133690506 | 133690654 |
| chr4 | 133830183 | 133830313 |
| chr4 | 134024805 | 134024929 |
| chr4 | 134159536 | 134159660 |
| chr4 | 134320732 | 134320858 |
| chr4 | 134396027 | 134396157 |
| chr4 | 134515355 | 134515507 |
| chr4 | 134670052 | 134670175 |
| chr4 | 134779871 | 134780003 |
| chr4 | 135006906 | 135007054 |
| chr4 | 135171933 | 135172075 |
| chr4 | 135234866 | 135234992 |
| chr4 | 135491826 | 135491950 |
| chr4 | 135590272 | 135590428 |
| chr4 | 135741525 | 135741655 |
| chr4 | 135928670 | 135928796 |
| chr4 | 135995393 | 135995519 |
| chr4 | 136164072 | 136164212 |
| chr4 | 136339651 | 136339777 |
| chr4 | 136452133 | 136452259 |
| chr4 | 136647469 | 136647599 |
| chr4 | 136824317 | 136824443 |
| chr4 | 137009666 | 137009798 |
| chr4 | 137166093 | 137166219 |
| chr4 | 137201801 | 137201937 |
| chr4 | 137395760 | 137395918 |
| chr4 | 137596492 | 137596618 |
| chr4 | 137757654 | 137757776 |
| chr4 | 137811691 | 137811831 |
| chr4 | 138005368 | 138005506 |
| chr4 | 138134082 | 138134222 |
| chr4 | 138252842 | 138252980 |
| chr4 | 138466151 | 138466299 |
| chr4 | 138626620 | 138626770 |
| chr4 | 138691316 | 138691440 |
| chr4 | 138876431 | 138876559 |
| chr4 | 139025984 | 139026112 |
| chr4 | 139223315 | 139223437 |
| chr4 | 139316215 | 139316351 |
| chr4 | 139494541 | 139494671 |
| chr4 | 139677908 | 139678032 |
| chr4 | 139808700 | 139808836 |
| chr4 | 139905895 | 139906021 |
| chr4 | 140115654 | 140115794 |
| chr4 | 140283885 | 140284029 |
| chr4 | 140345862 | 140345986 |
| chr4 | 140581844 | 140581966 |
| chr4 | 140714719 | 140714851 |
| chr4 | 140793457 | 140793579 |
| chr4 | 141035839 | 141035963 |
| chr4 | 141203449 | 141203627 |
| chr4 | 141349295 | 141349435 |
| chr4 | 141477982 | 141478128 |
| chr4 | 141673157 | 141673285 |
| chr4 | 141772277 | 141772403 |
| chr4 | 141923456 | 141923580 |
| chr4 | 142074893 | 142075021 |
| chr4 | 142154536 | 142154662 |
| chr4 | 142382090 | 142382212 |
| chr4 | 142548798 | 142548932 |
| chr4 | 142599162 | 142599304 |
| chr4 | 142795611 | 142795741 |
| chr4 | 142964028 | 142964150 |
| chr4 | 143128506 | 143128646 |
| chr4 | 143187214 | 143187340 |
| chr4 | 143407068 | 143407192 |
| chr4 | 143493159 | 143493299 |
| chr4 | 143747300 | 143747424 |
| chr4 | 143882959 | 143883097 |
| chr4 | 144020737 | 144020859 |
| chr4 | 144152807 | 144152957 |
| chr4 | 144376309 | 144376447 |
| chr4 | 144508470 | 144508622 |
| chr4 | 144535796 | 144535922 |
| chr4 | 144747117 | 144747241 |
| chr4 | 144868019 | 144868141 |
| chr4 | 145025996 | 145026130 |
| chr4 | 145239931 | 145240073 |
| chr4 | 145414710 | 145414838 |
| chr4 | 145560868 | 145560992 |
| chr4 | 145696950 | 145697072 |
| chr4 | 145780363 | 145780489 |
| chr4 | 145899009 | 145899139 |
| chr4 | 146102341 | 146102467 |
| chr4 | 146245970 | 146246096 |
| chr4 | 146469872 | 146469998 |
| chr4 | 146567532 | 146567654 |
| chr4 | 146662242 | 146662366 |
| chr4 | 146876463 | 146876605 |
| chr4 | 147055225 | 147055361 |
| chr4 | 147119027 | 147119193 |
| chr4 | 147238178 | 147238300 |
| chr4 | 147517593 | 147517733 |
| chr4 | 147556864 | 147556986 |
| chr4 | 147779945 | 147780069 |
| chr4 | 147873193 | 147873333 |
| chr4 | 148047460 | 148047612 |
| chr4 | 148245545 | 148245683 |
| chr4 | 148425320 | 148425474 |
| chr4 | 148486215 | 148486353 |
| chr4 | 148714496 | 148714622 |
| chr4 | 148844047 | 148844171 |
| chr4 | 148896992 | 148897114 |
| chr4 | 149089998 | 149090124 |
| chr4 | 149294248 | 149294374 |
| chr4 | 149448391 | 149448523 |
| chr4 | 149551652 | 149551776 |
| chr4 | 149706937 | 149707081 |
| chr4 | 149778293 | 149778439 |
| chr4 | 149950796 | 149950922 |
| chr4 | 150177288 | 150177410 |
| chr4 | 150237301 | 150237429 |
| chr4 | 150474642 | 150474772 |
| chr4 | 150563592 | 150563714 |
| chr4 | 150776928 | 150777050 |
| chr4 | 150975641 | 150975777 |
| chr4 | 151020620 | 151020744 |
| chr4 | 151247330 | 151247462 |
| chr4 | 151413585 | 151413707 |
| chr4 | 151509749 | 151509881 |
| chr4 | 151586516 | 151586682 |
| chr4 | 151751203 | 151751471 |
| chr4 | 151969984 | 151970109 |
| chr4 | 152142916 | 152143040 |
| chr4 | 152258387 | 152258539 |
| chr4 | 152383874 | 152384026 |
| chr4 | 152543693 | 152543817 |
| chr4 | 152707338 | 152707468 |
| chr4 | 152900410 | 152900534 |
| chr4 | 153039112 | 153039266 |
| chr4 | 153083201 | 153083329 |
| chr4 | 153256793 | 153256915 |
| chr4 | 153430317 | 153430473 |
| chr4 | 153621348 | 153621623 |
| chr4 | 153799965 | 153800117 |
| chr4 | 153967186 | 153967310 |
| chr4 | 154083856 | 154083982 |
| chr4 | 154176037 | 154176161 |
| chr4 | 154296308 | 154296456 |
| chr4 | 154448836 | 154448958 |
| chr4 | 154631990 | 154632138 |
| chr4 | 154749774 | 154749900 |
| chr4 | 154909195 | 154909339 |
| chr4 | 155056388 | 155056524 |
| chr4 | 155269221 | 155269347 |
| chr4 | 155451455 | 155451597 |
| chr4 | 155576808 | 155576930 |
| chr4 | 155752440 | 155752594 |
| chr4 | 155836112 | 155836240 |
| chr4 | 156011532 | 156011664 |
| chr4 | 156113781 | 156113939 |
| chr4 | 156292929 | 156293061 |
| chr4 | 156516666 | 156516792 |
| chr4 | 156619166 | 156619292 |
| chr4 | 156742077 | 156742205 |
| chr4 | 156833087 | 156833231 |
| chr4 | 157068005 | 157068129 |
| chr4 | 157137266 | 157137416 |
| chr4 | 157344737 | 157344865 |
| chr4 | 157555452 | 157555604 |
| chr4 | 157642634 | 157642756 |
| chr4 | 157836433 | 157836555 |
| chr4 | 158003739 | 158003879 |
| chr4 | 158115758 | 158115896 |
| chr4 | 158222635 | 158222777 |
| chr4 | 158415464 | 158415610 |
| chr4 | 158574297 | 158574431 |
| chr4 | 158682669 | 158682839 |
| chr4 | 158804912 | 158805036 |
| chr4 | 159033099 | 159033227 |
| chr4 | 159095836 | 159095968 |
| chr4 | 159342716 | 159342883 |
| chr4 | 159445280 | 159445404 |
| chr4 | 159672001 | 159672131 |
| chr4 | 159678527 | 159678651 |
| chr4 | 159851003 | 159851127 |
| chr4 | 159986020 | 159986142 |
| chr4 | 160167628 | 160167774 |
| chr4 | 160407768 | 160407920 |
| chr4 | 160538022 | 160538160 |
| chr4 | 160580207 | 160580335 |
| chr4 | 160771791 | 160771977 |
| chr4 | 160961064 | 160961248 |
| chr4 | 161094897 | 161095027 |
| chr4 | 161206542 | 161206666 |
| chr4 | 161399478 | 161399618 |
| chr4 | 161478801 | 161478923 |
| chr4 | 161660285 | 161660433 |
| chr4 | 161826903 | 161827033 |
| chr4 | 161934479 | 161934607 |
| chr4 | 162216436 | 162216560 |
| chr4 | 162319838 | 162319978 |
| chr4 | 162433311 | 162433455 |
| chr4 | 162625840 | 162625964 |
| chr4 | 162712016 | 162712138 |
| chr4 | 162933206 | 162933344 |
| chr4 | 162983421 | 162983567 |
| chr4 | 163242655 | 163242787 |
| chr4 | 163353181 | 163353305 |
| chr4 | 163541373 | 163541499 |
| chr4 | 163616114 | 163616252 |
| chr4 | 163802589 | 163802713 |
| chr4 | 163900270 | 163900396 |
| chr4 | 164099591 | 164099725 |
| chr4 | 164318124 | 164318246 |
| chr4 | 164437171 | 164437293 |
| chr4 | 164520978 | 164521114 |
| chr4 | 164693453 | 164693575 |
| chr4 | 164846403 | 164846527 |
| chr4 | 165077224 | 165077364 |
| chr4 | 165132708 | 165132836 |
| chr4 | 165259037 | 165259175 |
| chr4 | 165459281 | 165459417 |
| chr4 | 165633885 | 165634011 |
| chr4 | 165696952 | 165697098 |
| chr4 | 165910854 | 165910996 |
| chr4 | 165982312 | 165982446 |
| chr4 | 166129886 | 166130141 |
| chr4 | 166339086 | 166339226 |
| chr4 | 166523856 | 166524004 |
| chr4 | 166664985 | 166665113 |
| chr4 | 166847915 | 166848055 |
| chr4 | 167010633 | 167010755 |
| chr4 | 167163238 | 167163386 |
| chr4 | 167259167 | 167259293 |
| chr4 | 167437773 | 167437899 |
| chr4 | 167510446 | 167510578 |
| chr4 | 167755274 | 167755414 |
| chr4 | 167877923 | 167878075 |
| chr4 | 167964870 | 167964994 |
| chr4 | 168186020 | 168186150 |
| chr4 | 168307976 | 168308110 |
| chr4 | 168455308 | 168455470 |
| chr4 | 168592237 | 168592365 |
| chr4 | 168737558 | 168737688 |
| chr4 | 168866092 | 168866218 |
| chr4 | 169008841 | 169008971 |
| chr4 | 169156116 | 169156285 |
| chr4 | 169397973 | 169398097 |
| chr4 | 169447828 | 169447972 |
| chr4 | 169583970 | 169584094 |
| chr4 | 169844932 | 169845124 |
| chr4 | 169977850 | 169977974 |
| chr4 | 170068662 | 170068800 |
| chr4 | 170271334 | 170271470 |
| chr4 | 170331853 | 170331997 |
| chr4 | 170616411 | 170616535 |
| chr4 | 170651132 | 170651258 |
| chr4 | 170845783 | 170845907 |
| chr4 | 170927989 | 170928119 |
| chr4 | 171157890 | 171158012 |
| chr4 | 171336213 | 171336339 |
| chr4 | 171501421 | 171501567 |
| chr4 | 171552552 | 171552710 |
| chr4 | 171702471 | 171702595 |
| chr4 | 171888075 | 171888209 |
| chr4 | 172028648 | 172028790 |
| chr4 | 172228521 | 172228645 |
| chr4 | 172367060 | 172367210 |
| chr4 | 172459194 | 172459318 |
| chr4 | 172633795 | 172633947 |
| chr4 | 172862693 | 172862819 |
| chr4 | 172914721 | 172914845 |
| chr4 | 173041653 | 173041779 |
| chr4 | 173213207 | 173213339 |
| chr4 | 173365451 | 173365573 |
| chr4 | 173557111 | 173557251 |
| chr4 | 173672974 | 173673108 |
| chr4 | 173815181 | 173815339 |
| chr4 | 173928376 | 173928514 |
| chr4 | 174138815 | 174138949 |
| chr4 | 174375694 | 174375840 |
| chr4 | 174520324 | 174520456 |
| chr4 | 174550923 | 174551049 |
| chr4 | 174776744 | 174776872 |
| chr4 | 174891139 | 174891267 |
| chr4 | 174987581 | 174987731 |
| chr4 | 175212464 | 175212588 |
| chr4 | 175422110 | 175422238 |
| chr4 | 175500674 | 175500796 |
| chr4 | 175675406 | 175675530 |
| chr4 | 175756562 | 175756694 |
| chr4 | 175963324 | 175963448 |
| chr4 | 176142404 | 176142660 |
| chr4 | 176195312 | 176195434 |
| chr4 | 176361135 | 176361327 |
| chr4 | 176547554 | 176547700 |
| chr4 | 176700413 | 176700553 |
| chr4 | 176796951 | 176797081 |
| chr4 | 176992052 | 176992188 |
| chr4 | 177195105 | 177195233 |
| chr4 | 177242869 | 177243011 |
| chr4 | 177402138 | 177402272 |
| chr4 | 177530719 | 177530845 |
| chr4 | 177762218 | 177762342 |
| chr4 | 177925246 | 177925400 |
| chr4 | 177985872 | 177986012 |
| chr4 | 178190606 | 178190736 |
| chr4 | 178418853 | 178418981 |
| chr4 | 178570944 | 178571072 |
| chr4 | 178642293 | 178642425 |
| chr4 | 178735454 | 178735578 |
| chr4 | 178883281 | 178883427 |
| chr4 | 179176826 | 179176966 |
| chr4 | 179279321 | 179279457 |
| chr4 | 179342840 | 179342964 |
| chr4 | 179555550 | 179555676 |
| chr4 | 179666155 | 179666283 |
| chr4 | 179905135 | 179905257 |
| chr4 | 180050138 | 180050278 |
| chr4 | 180188069 | 180188193 |
| chr4 | 180230279 | 180230401 |
| chr4 | 180419301 | 180419437 |
| chr4 | 180548936 | 180549086 |
| chr4 | 180732058 | 180732184 |
| chr4 | 180831317 | 180831441 |
| chr4 | 181124217 | 181124349 |
| chr4 | 181260368 | 181260492 |
| chr4 | 181316127 | 181316273 |
| chr4 | 181549850 | 181549972 |
| chr4 | 181620338 | 181620488 |
| chr4 | 181754636 | 181754760 |
| chr4 | 181993188 | 181993318 |
| chr4 | 182030056 | 182030178 |
| chr4 | 182235387 | 182235531 |
| chr4 | 182344212 | 182344348 |
| chr4 | 182538903 | 182539057 |
| chr4 | 182724538 | 182724670 |
| chr4 | 182808655 | 182808779 |
| chr4 | 183054074 | 183054196 |
| chr4 | 183149603 | 183149745 |
| chr4 | 183229153 | 183229307 |
| chr4 | 183454086 | 183454220 |
| chr4 | 183632143 | 183632267 |
| chr4 | 183709804 | 183709928 |
| chr4 | 183913893 | 183914017 |
| chr4 | 184113727 | 184113885 |
| chr4 | 184190890 | 184191028 |
| chr4 | 184423822 | 184423950 |
| chr4 | 184499691 | 184499813 |
| chr4 | 184726113 | 184726259 |
| chr4 | 184757383 | 184757513 |
| chr4 | 184942318 | 184942466 |
| chr4 | 185054727 | 185054849 |
| chr4 | 185209839 | 185209969 |
| chr4 | 185398025 | 185398183 |
| chr4 | 185545939 | 185546063 |
| chr4 | 185721298 | 185721456 |
| chr4 | 185858947 | 185859075 |
| chr4 | 186044229 | 186044371 |
| chr4 | 186148579 | 186148711 |
| chr4 | 186349987 | 186350119 |
| chr4 | 186522901 | 186523023 |
| chr4 | 186589885 | 186590007 |
| chr4 | 186771565 | 186771693 |
| chr4 | 186842479 | 186842601 |
| chr4 | 187015051 | 187015177 |
| chr4 | 187192395 | 187192537 |
| chr4 | 187368404 | 187368534 |
| chr4 | 187484785 | 187484923 |
| chr4 | 187614398 | 187614522 |
| chr4 | 187793127 | 187793251 |
| chr4 | 187969402 | 187969540 |
| chr4 | 188159732 | 188159856 |
| chr4 | 188220189 | 188220313 |
| chr4 | 188430497 | 188430635 |
| chr4 | 188490685 | 188490815 |
| chr4 | 188734922 | 188735062 |
| chr4 | 188909380 | 188909520 |
| chr4 | 189019844 | 189019966 |
| chr4 | 189181781 | 189181903 |
| chr4 | 189315089 | 189315235 |
| chr4 | 189387404 | 189387526 |
| chr4 | 189633412 | 189633536 |
| chr4 | 189765883 | 189766029 |
| chr4 | 189926472 | 189926596 |
| chr4 | 190050550 | 190050672 |
| chr4 | 190232016 | 190232138 |
| chr4 | 190388486 | 190388612 |
| chr4 | 190539252 | 190539376 |
| chr4 | 190603392 | 190603520 |
| chr4 | 190734216 | 190734340 |
| chr5 | 100046 | 100180 |
| chr5 | 214053 | 214175 |
| chr5 | 342562 | 342690 |
| chr5 | 609476 | 609602 |
| chr5 | 653976 | 654104 |
| chr5 | 820388 | 820509 |
| chr5 | 965566 | 965696 |
| chr5 | 1130094 | 1130236 |
| chr5 | 1360049 | 1360179 |
| chr5 | 1531222 | 1531362 |
| chr5 | 1564919 | 1565044 |
| chr5 | 1826362 | 1826538 |
| chr5 | 1900579 | 1900715 |
| chr5 | 2023791 | 2023945 |
| chr5 | 2279967 | 2280091 |
| chr5 | 2418036 | 2418174 |
| chr5 | 2447132 | 2447272 |
| chr5 | 2622529 | 2622655 |
| chr5 | 2790023 | 2790145 |
| chr5 | 2999358 | 2999480 |
| chr5 | 3180734 | 3180858 |
| chr5 | 3258736 | 3258876 |
| chr5 | 3437255 | 3437377 |
| chr5 | 3543271 | 3543413 |
| chr5 | 3717817 | 3717957 |
| chr5 | 3865329 | 3865451 |
| chr5 | 4075108 | 4075266 |
| chr5 | 4136354 | 4136488 |
| chr5 | 4317159 | 4317303 |
| chr5 | 4416566 | 4416692 |
| chr5 | 4636169 | 4636319 |
| chr5 | 4696832 | 4696970 |
| chr5 | 4872437 | 4872559 |
| chr5 | 5054877 | 5055001 |
| chr5 | 5158888 | 5159016 |
| chr5 | 5372785 | 5372929 |
| chr5 | 5543490 | 5543630 |
| chr5 | 5703120 | 5703270 |
| chr5 | 5858834 | 5858958 |
| chr5 | 5937686 | 5937810 |
| chr5 | 6069779 | 6069907 |
| chr5 | 6208205 | 6208353 |
| chr5 | 6404842 | 6404966 |
| chr5 | 6522714 | 6522844 |
| chr5 | 6676873 | 6677005 |
| chr5 | 6893703 | 6893829 |
| chr5 | 7053428 | 7053558 |
| chr5 | 7202591 | 7202721 |
| chr5 | 7345767 | 7345897 |
| chr5 | 7422712 | 7422840 |
| chr5 | 7539650 | 7539796 |
| chr5 | 7780952 | 7781076 |
| chr5 | 7955619 | 7955741 |
| chr5 | 8083135 | 8083267 |
| chr5 | 8261696 | 8261826 |
| chr5 | 8408315 | 8408437 |
| chr5 | 8549345 | 8549495 |
| chr5 | 8685710 | 8685844 |
| chr5 | 8796144 | 8796268 |
| chr5 | 8907768 | 8907892 |
| chr5 | 9082463 | 9082605 |
| chr5 | 9302466 | 9302604 |
| chr5 | 9461958 | 9462090 |
| chr5 | 9627744 | 9627868 |
| chr5 | 9759456 | 9759580 |
| chr5 | 9877127 | 9877257 |
| chr5 | 9947560 | 9947694 |
| chr5 | 10105164 | 10105286 |
| chr5 | 10259327 | 10259451 |
| chr5 | 10444069 | 10444213 |
| chr5 | 10604937 | 10605063 |
| chr5 | 10804456 | 10804594 |
| chr5 | 10965501 | 10965643 |
| chr5 | 11022368 | 11022490 |
| chr5 | 11158373 | 11158503 |
| chr5 | 11378743 | 11378867 |
| chr5 | 11484372 | 11484510 |
| chr5 | 11696163 | 11696293 |
| chr5 | 11809623 | 11809876 |
| chr5 | 11992664 | 11992786 |
| chr5 | 12094952 | 12095076 |
| chr5 | 12266383 | 12266517 |
| chr5 | 12401092 | 12401216 |
| chr5 | 12599258 | 12599408 |
| chr5 | 12705615 | 12705755 |
| chr5 | 12906390 | 12906520 |
| chr5 | 12965524 | 12965658 |
| chr5 | 13207889 | 13208027 |
| chr5 | 13252738 | 13252886 |
| chr5 | 13443571 | 13443693 |
| chr5 | 13630122 | 13630256 |
| chr5 | 13730177 | 13730313 |
| chr5 | 13895531 | 13895677 |
| chr5 | 14009370 | 14009494 |
| chr5 | 14279500 | 14279624 |
| chr5 | 14360975 | 14361097 |
| chr5 | 14531096 | 14531220 |
| chr5 | 14599868 | 14600008 |
| chr5 | 14840610 | 14840754 |
| chr5 | 14890817 | 14890947 |
| chr5 | 15139067 | 15139205 |
| chr5 | 15195198 | 15195320 |
| chr5 | 15387331 | 15387573 |
| chr5 | 15520955 | 15521245 |
| chr5 | 15690093 | 15690225 |
| chr5 | 15791495 | 15791619 |
| chr5 | 15964424 | 15964562 |
| chr5 | 16102111 | 16102239 |
| chr5 | 16272593 | 16272725 |
| chr5 | 16494053 | 16494187 |
| chr5 | 16651058 | 16651180 |
| chr5 | 16727458 | 16727590 |
| chr5 | 16840603 | 16840729 |
| chr5 | 17012635 | 17012761 |
| chr5 | 17139063 | 17139189 |
| chr5 | 17290831 | 17290957 |
| chr5 | 17481547 | 17481672 |
| chr5 | 17653773 | 17653894 |
| chr5 | 17838651 | 17838783 |
| chr5 | 17970782 | 17970926 |
| chr5 | 18148482 | 18148606 |
| chr5 | 18306282 | 18306406 |
| chr5 | 18410352 | 18410476 |
| chr5 | 18490443 | 18490565 |
| chr5 | 18726940 | 18727064 |
| chr5 | 18809346 | 18809470 |
| chr5 | 19055587 | 19055711 |
| chr5 | 19174047 | 19174203 |
| chr5 | 19239427 | 19239561 |
| chr5 | 19448225 | 19448349 |
| chr5 | 19619784 | 19619928 |
| chr5 | 19715460 | 19715582 |
| chr5 | 19958967 | 19959119 |
| chr5 | 20023925 | 20024047 |
| chr5 | 20143876 | 20144028 |
| chr5 | 20420169 | 20420293 |
| chr5 | 20443612 | 20443754 |
| chr5 | 20592273 | 20592415 |
| chr5 | 20700836 | 20700960 |
| chr5 | 20850451 | 20850575 |
| chr5 | 20949854 | 20950024 |
| chr5 | 21052084 | 21052220 |
| chr5 | 21212286 | 21212422 |
| chr5 | 21309719 | 21309841 |
| chr5 | 21391753 | 21391875 |
| chr5 | 21480754 | 21480874 |
| chr5 | 21527878 | 21528064 |
| chr5 | 21660029 | 21660151 |
| chr5 | 21862940 | 21863098 |
| chr5 | 22086050 | 22086194 |
| chr5 | 22219794 | 22219924 |
| chr5 | 22288053 | 22288175 |
| chr5 | 22478730 | 22478862 |
| chr5 | 22646372 | 22646498 |
| chr5 | 22703241 | 22703373 |
| chr5 | 22941583 | 22941707 |
| chr5 | 23077313 | 23077483 |
| chr5 | 23210127 | 23210273 |
| chr5 | 23343934 | 23344070 |
| chr5 | 23574289 | 23574436 |
| chr5 | 23702555 | 23702681 |
| chr5 | 23876632 | 23876772 |
| chr5 | 23931262 | 23931408 |
| chr5 | 24110263 | 24110403 |
| chr5 | 24296652 | 24296786 |
| chr5 | 24359356 | 24359492 |
| chr5 | 24534487 | 24534611 |
| chr5 | 24782019 | 24782143 |
| chr5 | 24855095 | 24855237 |
| chr5 | 24967980 | 24968106 |
| chr5 | 25184430 | 25184560 |
| chr5 | 25247168 | 25247290 |
| chr5 | 25472899 | 25473047 |
| chr5 | 25621401 | 25621527 |
| chr5 | 25710676 | 25710810 |
| chr5 | 25885802 | 25885946 |
| chr5 | 26049023 | 26049153 |
| chr5 | 26165772 | 26165900 |
| chr5 | 26405763 | 26405907 |
| chr5 | 26539108 | 26539232 |
| chr5 | 26678467 | 26678619 |
| chr5 | 26758814 | 26758946 |
| chr5 | 26917950 | 26918072 |
| chr5 | 27061256 | 27061378 |
| chr5 | 27281422 | 27281544 |
| chr5 | 27357402 | 27357526 |
| chr5 | 27623422 | 27623548 |
| chr5 | 27714039 | 27714179 |
| chr5 | 27896699 | 27896823 |
| chr5 | 28046309 | 28046441 |
| chr5 | 28112059 | 28112187 |
| chr5 | 28367206 | 28367376 |
| chr5 | 28435891 | 28436027 |
| chr5 | 28655591 | 28655721 |
| chr5 | 28836095 | 28836239 |
| chr5 | 28932175 | 28932299 |
| chr5 | 29047751 | 29047879 |
| chr5 | 29162747 | 29162871 |
| chr5 | 29354751 | 29354893 |
| chr5 | 29484686 | 29484812 |
| chr5 | 29599604 | 29599726 |
| chr5 | 29772351 | 29772473 |
| chr5 | 29931681 | 29931821 |
| chr5 | 30059380 | 30059504 |
| chr5 | 30209129 | 30209259 |
| chr5 | 30368278 | 30368434 |
| chr5 | 30568345 | 30568467 |
| chr5 | 30772784 | 30772916 |
| chr5 | 30815811 | 30815961 |
| chr5 | 30939492 | 30939618 |
| chr5 | 31191330 | 31191454 |
| chr5 | 31254620 | 31254756 |
| chr5 | 31499680 | 31499816 |
| chr5 | 31607852 | 31607990 |
| chr5 | 31795300 | 31795422 |
| chr5 | 31938503 | 31938625 |
| chr5 | 32034226 | 32034358 |
| chr5 | 32159463 | 32159587 |
| chr5 | 32311451 | 32311587 |
| chr5 | 32457155 | 32457285 |
| chr5 | 32670724 | 32670848 |
| chr5 | 32746501 | 32746627 |
| chr5 | 32888742 | 32888870 |
| chr5 | 33169463 | 33169597 |
| chr5 | 33250469 | 33250611 |
| chr5 | 33401432 | 33401556 |
| chr5 | 33631306 | 33631430 |
| chr5 | 33761490 | 33761614 |
| chr5 | 33791011 | 33791157 |
| chr5 | 33972473 | 33972603 |
| chr5 | 34140815 | 34140939 |
| chr5 | 34168143 | 34168267 |
| chr5 | 34194316 | 34194436 |
| chr5 | 34322917 | 34323053 |
| chr5 | 34355638 | 34355761 |
| chr5 | 34440880 | 34441131 |
| chr5 | 34662457 | 34662585 |
| chr5 | 34797891 | 34798013 |
| chr5 | 34928640 | 34928764 |
| chr5 | 35015441 | 35015565 |
| chr5 | 35231935 | 35232073 |
| chr5 | 35419704 | 35419848 |
| chr5 | 35535085 | 35535211 |
| chr5 | 35636269 | 35636403 |
| chr5 | 35833194 | 35833318 |
| chr5 | 35937647 | 35937781 |
| chr5 | 36171372 | 36171494 |
| chr5 | 36312432 | 36312600 |
| chr5 | 36460376 | 36460510 |
| chr5 | 36538397 | 36538521 |
| chr5 | 36719939 | 36720063 |
| chr5 | 36935400 | 36935716 |
| chr5 | 36999908 | 37000032 |
| chr5 | 37131238 | 37131488 |
| chr5 | 37276288 | 37276414 |
| chr5 | 37406266 | 37406387 |
| chr5 | 37670906 | 37671042 |
| chr5 | 37720508 | 37720640 |
| chr5 | 37972564 | 37972692 |
| chr5 | 38087004 | 38087128 |
| chr5 | 38246591 | 38246713 |
| chr5 | 38346481 | 38346611 |
| chr5 | 38477789 | 38477911 |
| chr5 | 38596432 | 38596556 |
| chr5 | 38758254 | 38758378 |
| chr5 | 39030671 | 39030795 |
| chr5 | 39148591 | 39148727 |
| chr5 | 39247443 | 39247579 |
| chr5 | 39395339 | 39395477 |
| chr5 | 39512019 | 39512177 |
| chr5 | 39701305 | 39701443 |
| chr5 | 39906335 | 39906459 |
| chr5 | 39957407 | 39957539 |
| chr5 | 40192845 | 40192989 |
| chr5 | 40333661 | 40333803 |
| chr5 | 40488632 | 40488756 |
| chr5 | 40587724 | 40587868 |
| chr5 | 40785739 | 40785899 |
| chr5 | 40877352 | 40877474 |
| chr5 | 41094351 | 41094493 |
| chr5 | 41215184 | 41215334 |
| chr5 | 41304861 | 41304985 |
| chr5 | 41533581 | 41533721 |
| chr5 | 41726362 | 41726486 |
| chr5 | 41784050 | 41784171 |
| chr5 | 41918189 | 41918400 |
| chr5 | 42064412 | 42064538 |
| chr5 | 42190555 | 42190681 |
| chr5 | 42460616 | 42460768 |
| chr5 | 42589749 | 42589885 |
| chr5 | 42680006 | 42680150 |
| chr5 | 42805358 | 42805480 |
| chr5 | 42984545 | 42984669 |
| chr5 | 43149669 | 43149809 |
| chr5 | 43373261 | 43373387 |
| chr5 | 43459143 | 43459281 |
| chr5 | 43622014 | 43622191 |
| chr5 | 43709171 | 43709354 |
| chr5 | 43840815 | 43840936 |
| chr5 | 44098709 | 44098853 |
| chr5 | 44150313 | 44150439 |
| chr5 | 44361833 | 44361971 |
| chr5 | 44565576 | 44565700 |
| chr5 | 44646143 | 44646279 |
| chr5 | 44832168 | 44832314 |
| chr5 | 44927311 | 44927435 |
| chr5 | 45090869 | 45091004 |
| chr5 | 45227458 | 45227582 |
| chr5 | 45414478 | 45414632 |
| chr5 | 45622422 | 45622554 |
| chr5 | 45769329 | 45769467 |
| chr5 | 45905633 | 45905756 |
| chr5 | 45994129 | 45994273 |
| chr5 | 46097602 | 46097723 |
| chr5 | 46288878 | 46289026 |
| chr5 | 49562322 | 49562450 |
| chr5 | 49599110 | 49599242 |
| chr5 | 49653579 | 49653734 |
| chr5 | 49692361 | 49692487 |
| chr5 | 49767671 | 49767799 |
| chr5 | 49953920 | 49954204 |
| chr5 | 50112471 | 50112615 |
| chr5 | 50208011 | 50208187 |
| chr5 | 50419597 | 50419719 |
| chr5 | 50465171 | 50465307 |
| chr5 | 50598449 | 50598595 |
| chr5 | 50815475 | 50815599 |
| chr5 | 50928032 | 50928190 |
| chr5 | 51117462 | 51117598 |
| chr5 | 51292397 | 51292543 |
| chr5 | 51341607 | 51341755 |
| chr5 | 51590666 | 51590792 |
| chr5 | 51736217 | 51736353 |
| chr5 | 51919094 | 51919224 |
| chr5 | 51949718 | 51949844 |
| chr5 | 52142508 | 52142630 |
| chr5 | 52253566 | 52253716 |
| chr5 | 52533196 | 52533320 |
| chr5 | 52665655 | 52665781 |
| chr5 | 52710593 | 52710735 |
| chr5 | 52937785 | 52937925 |
| chr5 | 53109138 | 53109298 |
| chr5 | 53271350 | 53271474 |
| chr5 | 53420562 | 53420722 |
| chr5 | 53484170 | 53484300 |
| chr5 | 53709467 | 53709591 |
| chr5 | 53818455 | 53818589 |
| chr5 | 53983693 | 53983843 |
| chr5 | 54183114 | 54183252 |
| chr5 | 54301356 | 54301478 |
| chr5 | 54378130 | 54378272 |
| chr5 | 54631321 | 54631449 |
| chr5 | 54736952 | 54737084 |
| chr5 | 54875181 | 54875303 |
| chr5 | 55022923 | 55023083 |
| chr5 | 55169258 | 55169384 |
| chr5 | 55338739 | 55338869 |
| chr5 | 55523658 | 55523784 |
| chr5 | 55607456 | 55607608 |
| chr5 | 55834156 | 55834278 |
| chr5 | 55934864 | 55934990 |
| chr5 | 56019004 | 56019152 |
| chr5 | 56249199 | 56249339 |
| chr5 | 56292998 | 56293126 |
| chr5 | 56527579 | 56527709 |
| chr5 | 56628206 | 56628354 |
| chr5 | 56811661 | 56811789 |
| chr5 | 56944181 | 56944315 |
| chr5 | 57142203 | 57142325 |
| chr5 | 57269592 | 57269734 |
| chr5 | 57393598 | 57393726 |
| chr5 | 57620341 | 57620479 |
| chr5 | 57737130 | 57737256 |
| chr5 | 57819903 | 57820029 |
| chr5 | 58029513 | 58029639 |
| chr5 | 58139830 | 58139960 |
| chr5 | 58365159 | 58365283 |
| chr5 | 58436052 | 58436196 |
| chr5 | 58541508 | 58541638 |
| chr5 | 58831278 | 58831408 |
| chr5 | 58957583 | 58957715 |
| chr5 | 59096869 | 59097007 |
| chr5 | 59230337 | 59230459 |
| chr5 | 59323414 | 59323536 |
| chr5 | 59469837 | 59469979 |
| chr5 | 59669945 | 59670069 |
| chr5 | 59829087 | 59829229 |
| chr5 | 59974042 | 59974168 |
| chr5 | 60127634 | 60127784 |
| chr5 | 60305968 | 60306108 |
| chr5 | 60398208 | 60398336 |
| chr5 | 60588959 | 60589109 |
| chr5 | 60695135 | 60695259 |
| chr5 | 60892131 | 60892267 |
| chr5 | 61060611 | 61060733 |
| chr5 | 61113024 | 61113168 |
| chr5 | 61381421 | 61381551 |
| chr5 | 61443707 | 61443843 |
| chr5 | 61533737 | 61533859 |
| chr5 | 61533877 | 61534005 |
| chr5 | 61592980 | 61593104 |
| chr5 | 61753401 | 61753525 |
| chr5 | 61917902 | 61918024 |
| chr5 | 62007995 | 62008121 |
| chr5 | 62220415 | 62220557 |
| chr5 | 62427846 | 62427982 |
| chr5 | 62566446 | 62566588 |
| chr5 | 62670237 | 62670365 |
| chr5 | 62876908 | 62877036 |
| chr5 | 62954673 | 62954813 |
| chr5 | 63130936 | 63131060 |
| chr5 | 63325291 | 63325423 |
| chr5 | 63381593 | 63381721 |
| chr5 | 63566044 | 63566192 |
| chr5 | 63741985 | 63742123 |
| chr5 | 63882348 | 63882476 |
| chr5 | 64018743 | 64018889 |
| chr5 | 64150802 | 64150960 |
| chr5 | 64254142 | 64254280 |
| chr5 | 64455468 | 64455606 |
| chr5 | 64646057 | 64646181 |
| chr5 | 64726649 | 64726773 |
| chr5 | 64883781 | 64883903 |
| chr5 | 65131253 | 65131391 |
| chr5 | 65183194 | 65183322 |
| chr5 | 65377448 | 65377586 |
| chr5 | 65522816 | 65522976 |
| chr5 | 65732968 | 65733092 |
| chr5 | 65855816 | 65855940 |
| chr5 | 65895141 | 65895279 |
| chr5 | 66134631 | 66134753 |
| chr5 | 66260058 | 66260200 |
| chr5 | 66375798 | 66375938 |
| chr5 | 66568346 | 66568472 |
| chr5 | 66763978 | 66764100 |
| chr5 | 66936213 | 66936339 |
| chr5 | 67044052 | 67044178 |
| chr5 | 67129613 | 67129755 |
| chr5 | 67256515 | 67256639 |
| chr5 | 67442946 | 67443086 |
| chr5 | 67596889 | 67597015 |
| chr5 | 67707811 | 67707935 |
| chr5 | 67891672 | 67891853 |
| chr5 | 68025446 | 68025576 |
| chr5 | 68171494 | 68171638 |
| chr5 | 68296409 | 68296533 |
| chr5 | 68450865 | 68450989 |
| chr5 | 68716551 | 68716699 |
| chr5 | 68826685 | 68826936 |
| chr5 | 68932772 | 68932904 |
| chr5 | 68950805 | 68950937 |
| chr5 | 69035587 | 69035709 |
| chr5 | 69111199 | 69111321 |
| chr5 | 69193917 | 69194049 |
| chr5 | 69403942 | 69404065 |
| chr5 | 69512496 | 69512628 |
| chr5 | 69530546 | 69530678 |
| chr5 | 69615545 | 69615667 |
| chr5 | 69787601 | 69787733 |
| chr5 | 69808882 | 69809014 |
| chr5 | 69826923 | 69827055 |
| chr5 | 69910901 | 69911023 |
| chr5 | 69986100 | 69986222 |
| chr5 | 70069799 | 70069931 |
| chr5 | 70279409 | 70279532 |
| chr5 | 70309612 | 70309742 |
| chr5 | 70404294 | 70404418 |
| chr5 | 70512860 | 70512992 |
| chr5 | 70530881 | 70531013 |
| chr5 | 70614872 | 70614994 |
| chr5 | 70778971 | 70779109 |
| chr5 | 70884975 | 70885117 |
| chr5 | 70992727 | 70992855 |
| chr5 | 71245909 | 71246035 |
| chr5 | 71319206 | 71319328 |
| chr5 | 71554090 | 71554240 |
| chr5 | 71588756 | 71588894 |
| chr5 | 71744590 | 71744712 |
| chr5 | 71962312 | 71962452 |
| chr5 | 72125284 | 72125426 |
| chr5 | 72262428 | 72262564 |
| chr5 | 72388316 | 72388448 |
| chr5 | 72551285 | 72551423 |
| chr5 | 72741901 | 72742023 |
| chr5 | 72847511 | 72847685 |
| chr5 | 73055545 | 73055669 |
| chr5 | 73114947 | 73115085 |
| chr5 | 73356100 | 73356222 |
| chr5 | 73437495 | 73437619 |
| chr5 | 73539660 | 73539788 |
| chr5 | 73760698 | 73760836 |
| chr5 | 73851722 | 73851848 |
| chr5 | 74064226 | 74064350 |
| chr5 | 74160387 | 74160543 |
| chr5 | 74435284 | 74435412 |
| chr5 | 74540099 | 74540223 |
| chr5 | 74734306 | 74734440 |
| chr5 | 74875039 | 74875164 |
| chr5 | 74956481 | 74956605 |
| chr5 | 75097065 | 75097213 |
| chr5 | 75244453 | 75244589 |
| chr5 | 75388540 | 75388668 |
| chr5 | 75540832 | 75540970 |
| chr5 | 75722133 | 75722273 |
| chr5 | 75842293 | 75842423 |
| chr5 | 76059245 | 76059371 |
| chr5 | 76093165 | 76093289 |
| chr5 | 76276774 | 76276898 |
| chr5 | 76468275 | 76468405 |
| chr5 | 76581186 | 76581346 |
| chr5 | 76822372 | 76822514 |
| chr5 | 76968331 | 76968457 |
| chr5 | 77038762 | 77038886 |
| chr5 | 77177414 | 77177548 |
| chr5 | 77336524 | 77336662 |
| chr5 | 77513163 | 77513285 |
| chr5 | 77645187 | 77645317 |
| chr5 | 77770731 | 77770857 |
| chr5 | 77926991 | 77927119 |
| chr5 | 78074879 | 78075003 |
| chr5 | 78220906 | 78221050 |
| chr5 | 78409360 | 78409482 |
| chr5 | 78617478 | 78617614 |
| chr5 | 78682411 | 78682553 |
| chr5 | 78878305 | 78878433 |
| chr5 | 78945684 | 78945824 |
| chr5 | 79180157 | 79180289 |
| chr5 | 79288939 | 79289065 |
| chr5 | 79438331 | 79438479 |
| chr5 | 79622382 | 79622534 |
| chr5 | 79695284 | 79695408 |
| chr5 | 79928707 | 79928837 |
| chr5 | 80096692 | 80096822 |
| chr5 | 80253894 | 80254018 |
| chr5 | 80416635 | 80416761 |
| chr5 | 80526586 | 80526710 |
| chr5 | 80713008 | 80713150 |
| chr5 | 80757624 | 80757776 |
| chr5 | 80992459 | 80992583 |
| chr5 | 81175410 | 81175546 |
| chr5 | 81324329 | 81324453 |
| chr5 | 81371954 | 81372098 |
| chr5 | 81582939 | 81583061 |
| chr5 | 81753748 | 81753908 |
| chr5 | 81909290 | 81909416 |
| chr5 | 81955501 | 81955627 |
| chr5 | 82213475 | 82213599 |
| chr5 | 82380822 | 82380948 |
| chr5 | 82398036 | 82398196 |
| chr5 | 82584369 | 82584497 |
| chr5 | 82744473 | 82744597 |
| chr5 | 82914557 | 82914703 |
| chr5 | 82998893 | 82999049 |
| chr5 | 83213530 | 83213652 |
| chr5 | 83325803 | 83325925 |
| chr5 | 83483088 | 83483228 |
| chr5 | 83686327 | 83686451 |
| chr5 | 83792031 | 83792179 |
| chr5 | 84010292 | 84010414 |
| chr5 | 84177926 | 84178052 |
| chr5 | 84275510 | 84275650 |
| chr5 | 84361261 | 84361385 |
| chr5 | 84606450 | 84606580 |
| chr5 | 84712191 | 84712315 |
| chr5 | 84821248 | 84821372 |
| chr5 | 85049035 | 85049167 |
| chr5 | 85196532 | 85196672 |
| chr5 | 85261800 | 85261930 |
| chr5 | 85484392 | 85484542 |
| chr5 | 85650954 | 85651092 |
| chr5 | 85698200 | 85698326 |
| chr5 | 85880439 | 85880560 |
| chr5 | 85998586 | 85998708 |
| chr5 | 86270878 | 86271002 |
| chr5 | 86317061 | 86317332 |
| chr5 | 86571057 | 86571199 |
| chr5 | 86612485 | 86612609 |
| chr5 | 86829750 | 86829884 |
| chr5 | 87001814 | 87001952 |
| chr5 | 87054835 | 87054973 |
| chr5 | 87205455 | 87205579 |
| chr5 | 87407550 | 87407698 |
| chr5 | 87495299 | 87495425 |
| chr5 | 87678978 | 87679102 |
| chr5 | 87827204 | 87827338 |
| chr5 | 88048401 | 88048533 |
| chr5 | 88179364 | 88179498 |
| chr5 | 88269967 | 88270107 |
| chr5 | 88474052 | 88474178 |
| chr5 | 88604841 | 88604962 |
| chr5 | 88747929 | 88748051 |
| chr5 | 88912601 | 88912729 |
| chr5 | 89010908 | 89011056 |
| chr5 | 89230698 | 89230820 |
| chr5 | 89367171 | 89367295 |
| chr5 | 89442224 | 89442348 |
| chr5 | 89737129 | 89737275 |
| chr5 | 89776007 | 89776131 |
| chr5 | 89956811 | 89956935 |
| chr5 | 90057481 | 90057641 |
| chr5 | 90263773 | 90263903 |
| chr5 | 90466236 | 90466370 |
| chr5 | 90550325 | 90550459 |
| chr5 | 90724542 | 90724670 |
| chr5 | 90892273 | 90892429 |
| chr5 | 90968581 | 90968707 |
| chr5 | 91169304 | 91169438 |
| chr5 | 91379421 | 91379595 |
| chr5 | 91408266 | 91408392 |
| chr5 | 91634295 | 91634439 |
| chr5 | 91697915 | 91698041 |
| chr5 | 91970099 | 91970237 |
| chr5 | 92039943 | 92040065 |
| chr5 | 92187880 | 92188006 |
| chr5 | 92335326 | 92335468 |
| chr5 | 92468783 | 92468905 |
| chr5 | 92707675 | 92707815 |
| chr5 | 92866040 | 92866224 |
| chr5 | 92950585 | 92950725 |
| chr5 | 93071772 | 93071900 |
| chr5 | 93227460 | 93227721 |
| chr5 | 93414071 | 93414338 |
| chr5 | 93522383 | 93522649 |
| chr5 | 93773366 | 93773498 |
| chr5 | 93896068 | 93896202 |
| chr5 | 94008879 | 94009021 |
| chr5 | 94111251 | 94111385 |
| chr5 | 94239986 | 94240124 |
| chr5 | 94469652 | 94469780 |
| chr5 | 94554201 | 94554323 |
| chr5 | 94726107 | 94726245 |
| chr5 | 94982655 | 94982781 |
| chr5 | 95002031 | 95002177 |
| chr5 | 95177817 | 95177955 |
| chr5 | 95367004 | 95367136 |
| chr5 | 95584003 | 95584137 |
| chr5 | 95685145 | 95685271 |
| chr5 | 95875440 | 95875564 |
| chr5 | 95924484 | 95924636 |
| chr5 | 96112864 | 96113006 |
| chr5 | 96250421 | 96250545 |
| chr5 | 96427005 | 96427141 |
| chr5 | 96561270 | 96561412 |
| chr5 | 96703870 | 96704022 |
| chr5 | 96920910 | 96921036 |
| chr5 | 96950976 | 96951106 |
| chr5 | 97129594 | 97129746 |
| chr5 | 97260280 | 97260412 |
| chr5 | 97481550 | 97481684 |
| chr5 | 97646069 | 97646195 |
| chr5 | 97797633 | 97797789 |
| chr5 | 97859810 | 97859956 |
| chr5 | 98099882 | 98100014 |
| chr5 | 98267055 | 98267177 |
| chr5 | 98430980 | 98431118 |
| chr5 | 98524784 | 98524922 |
| chr5 | 98668806 | 98668950 |
| chr5 | 98795906 | 98796039 |
| chr5 | 98923985 | 98924111 |
| chr5 | 99110284 | 99110436 |
| chr5 | 99206431 | 99206571 |
| chr5 | 99352419 | 99352541 |
| chr5 | 99583691 | 99583841 |
| chr5 | 99664557 | 99664681 |
| chr5 | 99913127 | 99913257 |
| chr5 | 99969680 | 99969830 |
| chr5 | 100188863 | 100189041 |
| chr5 | 100376449 | 100376603 |
| chr5 | 100494137 | 100494261 |
| chr5 | 100546695 | 100546827 |
| chr5 | 100768381 | 100768539 |
| chr5 | 100931221 | 100931349 |
| chr5 | 101038596 | 101038852 |
| chr5 | 101283200 | 101283338 |
| chr5 | 101302338 | 101302486 |
| chr5 | 101474806 | 101474932 |
| chr5 | 101707936 | 101708072 |
| chr5 | 101877441 | 101877577 |
| chr5 | 102004785 | 102004937 |
| chr5 | 102168848 | 102168972 |
| chr5 | 102251336 | 102251470 |
| chr5 | 102460659 | 102460797 |
| chr5 | 102608759 | 102608881 |
| chr5 | 102733263 | 102733387 |
| chr5 | 102892172 | 102892312 |
| chr5 | 103073487 | 103073609 |
| chr5 | 103220370 | 103220510 |
| chr5 | 103331756 | 103331904 |
| chr5 | 103491689 | 103491811 |
| chr5 | 103576744 | 103576866 |
| chr5 | 103812967 | 103813095 |
| chr5 | 103864365 | 103864511 |
| chr5 | 104021496 | 104021636 |
| chr5 | 104283392 | 104283530 |
| chr5 | 104312426 | 104312562 |
| chr5 | 104528037 | 104528173 |
| chr5 | 104594797 | 104594933 |
| chr5 | 104788601 | 104788727 |
| chr5 | 104995261 | 104995405 |
| chr5 | 105145781 | 105145907 |
| chr5 | 105217310 | 105217434 |
| chr5 | 105376064 | 105376198 |
| chr5 | 105540870 | 105541022 |
| chr5 | 105641971 | 105642115 |
| chr5 | 105790143 | 105790273 |
| chr5 | 106047878 | 106048012 |
| chr5 | 106163534 | 106163658 |
| chr5 | 106303256 | 106303382 |
| chr5 | 106415320 | 106415446 |
| chr5 | 106669904 | 106670034 |
| chr5 | 106707741 | 106707863 |
| chr5 | 106919295 | 106919419 |
| chr5 | 107113247 | 107113369 |
| chr5 | 107260188 | 107260332 |
| chr5 | 107366168 | 107366306 |
| chr5 | 107494977 | 107495123 |
| chr5 | 107722113 | 107722237 |
| chr5 | 107857797 | 107857921 |
| chr5 | 107980774 | 107980902 |
| chr5 | 108147996 | 108148130 |
| chr5 | 108297780 | 108297906 |
| chr5 | 108450190 | 108450324 |
| chr5 | 108602225 | 108602357 |
| chr5 | 108648143 | 108648265 |
| chr5 | 108861270 | 108861398 |
| chr5 | 109023320 | 109023462 |
| chr5 | 109199357 | 109199479 |
| chr5 | 109276305 | 109276441 |
| chr5 | 109398806 | 109398944 |
| chr5 | 109687653 | 109687775 |
| chr5 | 109800285 | 109800413 |
| chr5 | 109879133 | 109879259 |
| chr5 | 110105075 | 110105245 |
| chr5 | 110259150 | 110259278 |
| chr5 | 110358171 | 110358295 |
| chr5 | 110489985 | 110490123 |
| chr5 | 110621181 | 110621309 |
| chr5 | 110859707 | 110859855 |
| chr5 | 110926018 | 110926150 |
| chr5 | 111040502 | 111040624 |
| chr5 | 111310047 | 111310187 |
| chr5 | 111370984 | 111371112 |
| chr5 | 111584175 | 111584317 |
| chr5 | 111711408 | 111711529 |
| chr5 | 111928363 | 111928507 |
| chr5 | 112054469 | 112054591 |
| chr5 | 112172151 | 112172307 |
| chr5 | 112288927 | 112289049 |
| chr5 | 112459969 | 112460097 |
| chr5 | 112563598 | 112563752 |
| chr5 | 112817932 | 112818068 |
| chr5 | 112911623 | 112911753 |
| chr5 | 113085613 | 113085755 |
| chr5 | 113238194 | 113238326 |
| chr5 | 113337645 | 113337789 |
| chr5 | 113440826 | 113440956 |
| chr5 | 113601329 | 113601453 |
| chr5 | 113769892 | 113770024 |
| chr5 | 114004078 | 114004222 |
| chr5 | 114095232 | 114095354 |
| chr5 | 114324864 | 114324990 |
| chr5 | 114475094 | 114475246 |
| chr5 | 114527330 | 114527480 |
| chr5 | 114743435 | 114743561 |
| chr5 | 114890451 | 114890587 |
| chr5 | 114940247 | 114940379 |
| chr5 | 115122773 | 115122905 |
| chr5 | 115356744 | 115356872 |
| chr5 | 115469074 | 115469216 |
| chr5 | 115629449 | 115629571 |
| chr5 | 115775456 | 115775610 |
| chr5 | 115952192 | 115952332 |
| chr5 | 116126130 | 116126266 |
| chr5 | 116153143 | 116153285 |
| chr5 | 116376630 | 116376774 |
| chr5 | 116508637 | 116508783 |
| chr5 | 116601511 | 116601639 |
| chr5 | 116861751 | 116861887 |
| chr5 | 117016207 | 117016341 |
| chr5 | 117112244 | 117112366 |
| chr5 | 117246670 | 117246810 |
| chr5 | 117420113 | 117420386 |
| chr5 | 117614249 | 117614379 |
| chr5 | 117717090 | 117717212 |
| chr5 | 117804705 | 117804976 |
| chr5 | 118027522 | 118027646 |
| chr5 | 118166636 | 118166776 |
| chr5 | 118276102 | 118276223 |
| chr5 | 118497777 | 118497946 |
| chr5 | 118636153 | 118636295 |
| chr5 | 118730823 | 118730951 |
| chr5 | 118916182 | 118916306 |
| chr5 | 119093636 | 119093782 |
| chr5 | 119146044 | 119146172 |
| chr5 | 119303164 | 119303292 |
| chr5 | 119460211 | 119460335 |
| chr5 | 119626336 | 119626458 |
| chr5 | 119772353 | 119772475 |
| chr5 | 120018163 | 120018297 |
| chr5 | 120186469 | 120186615 |
| chr5 | 120316959 | 120317101 |
| chr5 | 120438255 | 120438385 |
| chr5 | 120565516 | 120565654 |
| chr5 | 120708658 | 120708800 |
| chr5 | 120936563 | 120936691 |
| chr5 | 121034364 | 121034512 |
| chr5 | 121101922 | 121102047 |
| chr5 | 121261569 | 121261691 |
| chr5 | 121436501 | 121436651 |
| chr5 | 121678162 | 121678300 |
| chr5 | 121768581 | 121768719 |
| chr5 | 121877137 | 121877261 |
| chr5 | 122042322 | 122042468 |
| chr5 | 122233536 | 122233670 |
| chr5 | 122362046 | 122362190 |
| chr5 | 122447700 | 122447824 |
| chr5 | 122675377 | 122675519 |
| chr5 | 122774143 | 122774273 |
| chr5 | 123002661 | 123002785 |
| chr5 | 123181061 | 123181187 |
| chr5 | 123329099 | 123329223 |
| chr5 | 123452715 | 123452841 |
| chr5 | 123620024 | 123620170 |
| chr5 | 123707186 | 123707308 |
| chr5 | 123932728 | 123932872 |
| chr5 | 124050359 | 124050481 |
| chr5 | 124158900 | 124159024 |
| chr5 | 124359450 | 124359574 |
| chr5 | 124513195 | 124513333 |
| chr5 | 124584948 | 124585070 |
| chr5 | 124767272 | 124767394 |
| chr5 | 124914476 | 124914600 |
| chr5 | 125088847 | 125088991 |
| chr5 | 125141537 | 125141667 |
| chr5 | 125311985 | 125312109 |
| chr5 | 125506962 | 125507102 |
| chr5 | 125636032 | 125636154 |
| chr5 | 125818833 | 125818957 |
| chr5 | 125895437 | 125895597 |
| chr5 | 126114626 | 126114873 |
| chr5 | 126254390 | 126254534 |
| chr5 | 126462871 | 126463015 |
| chr5 | 126503069 | 126503193 |
| chr5 | 126772988 | 126773114 |
| chr5 | 126892701 | 126892837 |
| chr5 | 127008631 | 127008787 |
| chr5 | 127113856 | 127113984 |
| chr5 | 127292644 | 127292788 |
| chr5 | 127468125 | 127468247 |
| chr5 | 127549697 | 127549819 |
| chr5 | 127835562 | 127835698 |
| chr5 | 127843482 | 127843614 |
| chr5 | 128001096 | 128001218 |
| chr5 | 128150161 | 128150295 |
| chr5 | 128350179 | 128350319 |
| chr5 | 128500676 | 128500826 |
| chr5 | 128737654 | 128737784 |
| chr5 | 128813437 | 128813563 |
| chr5 | 128945496 | 128945620 |
| chr5 | 129085978 | 129086116 |
| chr5 | 129293371 | 129293497 |
| chr5 | 129429553 | 129429697 |
| chr5 | 129632976 | 129633106 |
| chr5 | 129758600 | 129758724 |
| chr5 | 129898431 | 129898577 |
| chr5 | 129940012 | 129940142 |
| chr5 | 130180838 | 130180970 |
| chr5 | 130362391 | 130362521 |
| chr5 | 130414724 | 130414854 |
| chr5 | 130553336 | 130553458 |
| chr5 | 130693046 | 130693172 |
| chr5 | 130894523 | 130894691 |
| chr5 | 131038870 | 131039028 |
| chr5 | 131252334 | 131252470 |
| chr5 | 131301537 | 131301663 |
| chr5 | 131501855 | 131501983 |
| chr5 | 131618539 | 131618673 |
| chr5 | 131791758 | 131791894 |
| chr5 | 131999404 | 131999560 |
| chr5 | 132174450 | 132174580 |
| chr5 | 132311540 | 132311664 |
| chr5 | 132358830 | 132358958 |
| chr5 | 132580972 | 132581096 |
| chr5 | 132755393 | 132755537 |
| chr5 | 132821542 | 132821676 |
| chr5 | 132954335 | 132954459 |
| chr5 | 133110418 | 133110570 |
| chr5 | 133355664 | 133355798 |
| chr5 | 133504801 | 133504923 |
| chr5 | 133655380 | 133655504 |
| chr5 | 133774106 | 133774250 |
| chr5 | 133864553 | 133864685 |
| chr5 | 134058408 | 134058534 |
| chr5 | 134272105 | 134272227 |
| chr5 | 134331076 | 134331329 |
| chr5 | 134440364 | 134440514 |
| chr5 | 134648763 | 134648905 |
| chr5 | 134864178 | 134864300 |
| chr5 | 134959432 | 134959556 |
| chr5 | 135073993 | 135074115 |
| chr5 | 135261248 | 135261370 |
| chr5 | 135486052 | 135486188 |
| chr5 | 135516220 | 135516352 |
| chr5 | 135778894 | 135779024 |
| chr5 | 135864483 | 135864625 |
| chr5 | 136080493 | 136080617 |
| chr5 | 136141634 | 136141774 |
| chr5 | 136273713 | 136273839 |
| chr5 | 136481808 | 136481946 |
| chr5 | 136614544 | 136614678 |
| chr5 | 136732312 | 136732448 |
| chr5 | 136869694 | 136869826 |
| chr5 | 137109340 | 137109460 |
| chr5 | 137216014 | 137216205 |
| chr5 | 137417815 | 137417941 |
| chr5 | 137568829 | 137568953 |
| chr5 | 137722746 | 137722874 |
| chr5 | 137876866 | 137876992 |
| chr5 | 137934535 | 137934657 |
| chr5 | 138167433 | 138167569 |
| chr5 | 138216195 | 138216333 |
| chr5 | 138444880 | 138445014 |
| chr5 | 138637503 | 138637627 |
| chr5 | 138747400 | 138747548 |
| chr5 | 138923399 | 138923520 |
| chr5 | 139012024 | 139012144 |
| chr5 | 139154724 | 139154850 |
| chr5 | 139375878 | 139376002 |
| chr5 | 139500918 | 139501170 |
| chr5 | 139664631 | 139664753 |
| chr5 | 139790274 | 139790428 |
| chr5 | 139890999 | 139891125 |
| chr5 | 140086960 | 140087086 |
| chr5 | 140234986 | 140235130 |
| chr5 | 140408083 | 140408231 |
| chr5 | 140482813 | 140482943 |
| chr5 | 140721378 | 140721500 |
| chr5 | 140862772 | 140862896 |
| chr5 | 141037574 | 141037734 |
| chr5 | 141156233 | 141156371 |
| chr5 | 141277128 | 141277260 |
| chr5 | 141414299 | 141414425 |
| chr5 | 141508404 | 141508536 |
| chr5 | 141642876 | 141643004 |
| chr5 | 141836727 | 141836851 |
| chr5 | 142055915 | 142056045 |
| chr5 | 142168387 | 142168543 |
| chr5 | 142319221 | 142319349 |
| chr5 | 142407744 | 142407888 |
| chr5 | 142616396 | 142616534 |
| chr5 | 142786659 | 142786783 |
| chr5 | 142959609 | 142959731 |
| chr5 | 143055384 | 143055520 |
| chr5 | 143238784 | 143238918 |
| chr5 | 143390667 | 143390823 |
| chr5 | 143525196 | 143525336 |
| chr5 | 143672077 | 143672205 |
| chr5 | 143842259 | 143842413 |
| chr5 | 143898152 | 143898274 |
| chr5 | 144101718 | 144101848 |
| chr5 | 144266930 | 144267054 |
| chr5 | 144461536 | 144461670 |
| chr5 | 144620704 | 144620862 |
| chr5 | 144764547 | 144764679 |
| chr5 | 144874881 | 144875007 |
| chr5 | 145007071 | 145007193 |
| chr5 | 145235401 | 145235523 |
| chr5 | 145323492 | 145323616 |
| chr5 | 145409760 | 145409902 |
| chr5 | 145663773 | 145663909 |
| chr5 | 145764714 | 145764854 |
| chr5 | 145839089 | 145839243 |
| chr5 | 146010289 | 146010411 |
| chr5 | 146158849 | 146158977 |
| chr5 | 146300100 | 146300240 |
| chr5 | 146580153 | 146580277 |
| chr5 | 146649345 | 146649473 |
| chr5 | 146760506 | 146760630 |
| chr5 | 147021746 | 147021872 |
| chr5 | 147073665 | 147073789 |
| chr5 | 147207991 | 147208115 |
| chr5 | 147346008 | 147346172 |
| chr5 | 147489427 | 147489565 |
| chr5 | 147682058 | 147682200 |
| chr5 | 147864983 | 147865113 |
| chr5 | 148073646 | 148073786 |
| chr5 | 148191518 | 148191650 |
| chr5 | 148319293 | 148319425 |
| chr5 | 148513228 | 148513382 |
| chr5 | 148557985 | 148558129 |
| chr5 | 148822074 | 148822200 |
| chr5 | 148873550 | 148873678 |
| chr5 | 149114172 | 149114312 |
| chr5 | 149176883 | 149177033 |
| chr5 | 149350570 | 149350700 |
| chr5 | 149466979 | 149467101 |
| chr5 | 149704746 | 149704880 |
| chr5 | 149744152 | 149744274 |
| chr5 | 149897219 | 149897379 |
| chr5 | 150130790 | 150130912 |
| chr5 | 150291317 | 150291459 |
| chr5 | 150426778 | 150426902 |
| chr5 | 150507371 | 150507523 |
| chr5 | 150742883 | 150743005 |
| chr5 | 150831935 | 150832063 |
| chr5 | 151056629 | 151056755 |
| chr5 | 151182582 | 151182708 |
| chr5 | 151255813 | 151255967 |
| chr5 | 151396713 | 151396849 |
| chr5 | 151571656 | 151571784 |
| chr5 | 151814408 | 151814580 |
| chr5 | 151930223 | 151930353 |
| chr5 | 152002249 | 152002381 |
| chr5 | 152185884 | 152186006 |
| chr5 | 152332618 | 152332742 |
| chr5 | 152586038 | 152586184 |
| chr5 | 152726982 | 152727122 |
| chr5 | 152861954 | 152862078 |
| chr5 | 152905137 | 152905269 |
| chr5 | 153064904 | 153065060 |
| chr5 | 153335490 | 153335618 |
| chr5 | 153446735 | 153446877 |
| chr5 | 153495781 | 153495921 |
| chr5 | 153647153 | 153647277 |
| chr5 | 153824158 | 153824302 |
| chr5 | 154054791 | 154054933 |
| chr5 | 154182146 | 154182270 |
| chr5 | 154324800 | 154324983 |
| chr5 | 154457825 | 154457949 |
| chr5 | 154575351 | 154575501 |
| chr5 | 154809427 | 154809583 |
| chr5 | 154960494 | 154960622 |
| chr5 | 155128488 | 155128618 |
| chr5 | 155191667 | 155191792 |
| chr5 | 155359559 | 155359685 |
| chr5 | 155479955 | 155480083 |
| chr5 | 155647198 | 155647322 |
| chr5 | 155779265 | 155779387 |
| chr5 | 155987850 | 155987978 |
| chr5 | 156043157 | 156043285 |
| chr5 | 156285036 | 156285157 |
| chr5 | 156465613 | 156465749 |
| chr5 | 156624001 | 156624135 |
| chr5 | 156779122 | 156779250 |
| chr5 | 156821157 | 156821287 |
| chr5 | 157075391 | 157075523 |
| chr5 | 157182845 | 157182979 |
| chr5 | 157347047 | 157347191 |
| chr5 | 157506660 | 157506782 |
| chr5 | 157655194 | 157655336 |
| chr5 | 157713231 | 157713355 |
| chr5 | 157922096 | 157922236 |
| chr5 | 158103341 | 158103477 |
| chr5 | 158257986 | 158258136 |
| chr5 | 158394304 | 158394430 |
| chr5 | 158470009 | 158470149 |
| chr5 | 158722822 | 158722944 |
| chr5 | 158770198 | 158770342 |
| chr5 | 159001375 | 159001499 |
| chr5 | 159098302 | 159098424 |
| chr5 | 159283605 | 159283729 |
| chr5 | 159445817 | 159445951 |
| chr5 | 159633613 | 159633753 |
| chr5 | 159667529 | 159667653 |
| chr5 | 159812658 | 159812806 |
| chr5 | 159960790 | 159960918 |
| chr5 | 160125032 | 160125160 |
| chr5 | 160283207 | 160283333 |
| chr5 | 160488316 | 160488452 |
| chr5 | 160591446 | 160591594 |
| chr5 | 160825063 | 160825205 |
| chr5 | 160952943 | 160953077 |
| chr5 | 161011418 | 161011562 |
| chr5 | 161158692 | 161158832 |
| chr5 | 161400986 | 161401122 |
| chr5 | 161582577 | 161582709 |
| chr5 | 161621781 | 161621903 |
| chr5 | 161789926 | 161790050 |
| chr5 | 162019810 | 162019948 |
| chr5 | 162124144 | 162124272 |
| chr5 | 162233721 | 162233847 |
| chr5 | 162357617 | 162357745 |
| chr5 | 162600866 | 162600994 |
| chr5 | 162671963 | 162672105 |
| chr5 | 162808930 | 162809064 |
| chr5 | 163074472 | 163074614 |
| chr5 | 163200463 | 163200599 |
| chr5 | 163263013 | 163263157 |
| chr5 | 163530498 | 163530662 |
| chr5 | 163632599 | 163632733 |
| chr5 | 163729754 | 163729898 |
| chr5 | 163875935 | 163876057 |
| chr5 | 164019852 | 164019988 |
| chr5 | 164279383 | 164279515 |
| chr5 | 164365262 | 164365390 |
| chr5 | 164473555 | 164473699 |
| chr5 | 164594870 | 164594994 |
| chr5 | 164884448 | 164884572 |
| chr5 | 164960052 | 164960174 |
| chr5 | 165155329 | 165155467 |
| chr5 | 165265307 | 165265439 |
| chr5 | 165367177 | 165367301 |
| chr5 | 165552481 | 165552637 |
| chr5 | 165728158 | 165728290 |
| chr5 | 165902295 | 165902419 |
| chr5 | 166041584 | 166041710 |
| chr5 | 166134400 | 166134574 |
| chr5 | 166380151 | 166380289 |
| chr5 | 166458942 | 166459070 |
| chr5 | 166666559 | 166666695 |
| chr5 | 166721112 | 166721256 |
| chr5 | 166874592 | 166874714 |
| chr5 | 167055862 | 167055986 |
| chr5 | 167157999 | 167158137 |
| chr5 | 167402961 | 167403083 |
| chr5 | 167496799 | 167496937 |
| chr5 | 167706377 | 167706527 |
| chr5 | 167793067 | 167793193 |
| chr5 | 167965699 | 167965835 |
| chr5 | 168073313 | 168073441 |
| chr5 | 168267632 | 168267754 |
| chr5 | 168454901 | 168455025 |
| chr5 | 168498609 | 168498731 |
| chr5 | 168768898 | 168769022 |
| chr5 | 168839030 | 168839168 |
| chr5 | 168971469 | 168971595 |
| chr5 | 169157419 | 169157563 |
| chr5 | 169317548 | 169317700 |
| chr5 | 169420034 | 169420158 |
| chr5 | 169658181 | 169658307 |
| chr5 | 169765389 | 169765513 |
| chr5 | 169921857 | 169921979 |
| chr5 | 170013158 | 170013282 |
| chr5 | 170240108 | 170240258 |
| chr5 | 170380111 | 170380235 |
| chr5 | 170573688 | 170573812 |
| chr5 | 170674167 | 170674299 |
| chr5 | 170863473 | 170863597 |
| chr5 | 171033058 | 171033218 |
| chr5 | 171136465 | 171136599 |
| chr5 | 171285587 | 171285708 |
| chr5 | 171390428 | 171390846 |
| chr5 | 171632538 | 171632668 |
| chr5 | 171699232 | 171699384 |
| chr5 | 171822614 | 171822738 |
| chr5 | 171969453 | 171969577 |
| chr5 | 172201900 | 172202022 |
| chr5 | 172348546 | 172348672 |
| chr5 | 172412513 | 172412637 |
| chr5 | 172546212 | 172546354 |
| chr5 | 172715147 | 172715269 |
| chr5 | 172875942 | 172876086 |
| chr5 | 172990440 | 172990562 |
| chr5 | 173227245 | 173227377 |
| chr5 | 173334730 | 173334880 |
| chr5 | 173500370 | 173500631 |
| chr5 | 173675160 | 173675286 |
| chr5 | 173760170 | 173760330 |
| chr5 | 174027571 | 174027719 |
| chr5 | 174186689 | 174186815 |
| chr5 | 174309120 | 174309258 |
| chr5 | 174384316 | 174384458 |
| chr5 | 174567514 | 174567658 |
| chr5 | 174710321 | 174710447 |
| chr5 | 174861152 | 174861274 |
| chr5 | 174959826 | 174959958 |
| chr5 | 175186298 | 175186434 |
| chr5 | 175349491 | 175349767 |
| chr5 | 175485979 | 175486105 |
| chr5 | 175486637 | 175486759 |
| chr5 | 175614898 | 175615019 |
| chr5 | 175750441 | 175750565 |
| chr5 | 175889893 | 175890029 |
| chr5 | 176085438 | 176085602 |
| chr5 | 176235039 | 176235163 |
| chr5 | 176334515 | 176334659 |
| chr5 | 176563380 | 176563518 |
| chr5 | 176697760 | 176697894 |
| chr5 | 176847929 | 176848063 |
| chr5 | 176890556 | 176890706 |
| chr5 | 177046807 | 177046927 |
| chr5 | 177211331 | 177211453 |
| chr5 | 177211986 | 177212112 |
| chr5 | 177377801 | 177377935 |
| chr5 | 177431684 | 177431810 |
| chr5 | 177432343 | 177432465 |
| chr5 | 177560265 | 177560395 |
| chr5 | 177663609 | 177663745 |
| chr5 | 177840302 | 177840436 |
| chr5 | 178086439 | 178086569 |
| chr5 | 178103317 | 178103467 |
| chr5 | 178274480 | 178274606 |
| chr5 | 178426066 | 178426198 |
| chr5 | 178604317 | 178604465 |
| chr5 | 178717485 | 178717617 |
| chr5 | 178974925 | 178975075 |
| chr5 | 179044216 | 179044342 |
| chr5 | 179240826 | 179240948 |
| chr5 | 179350086 | 179350218 |
| chr5 | 179574840 | 179574964 |
| chr5 | 179657366 | 179657510 |
| chr5 | 179867751 | 179867875 |
| chr5 | 179937384 | 179937508 |
| chr5 | 180157153 | 180157279 |
| chr5 | 180314261 | 180314399 |
| chr5 | 180449821 | 180449943 |
| chr5 | 180600113 | 180600257 |
| chr5 | 180645926 | 180646048 |
| chr5 | 180723027 | 180723157 |
| chr6 | 153655 | 153785 |
| chr6 | 174677 | 174798 |
| chr6 | 455345 | 455467 |
| chr6 | 544161 | 544295 |
| chr6 | 744946 | 745084 |
| chr6 | 846881 | 847003 |
| chr6 | 1063795 | 1063921 |
| chr6 | 1163471 | 1163611 |
| chr6 | 1365192 | 1365318 |
| chr6 | 1451484 | 1451610 |
| chr6 | 1554266 | 1554404 |
| chr6 | 1784048 | 1784180 |
| chr6 | 1885882 | 1886060 |
| chr6 | 2095975 | 2096123 |
| chr6 | 2162564 | 2162710 |
| chr6 | 2346207 | 2346355 |
| chr6 | 2473168 | 2473292 |
| chr6 | 2617967 | 2618107 |
| chr6 | 2828089 | 2828219 |
| chr6 | 2914573 | 2914697 |
| chr6 | 3041475 | 3041607 |
| chr6 | 3292281 | 3292405 |
| chr6 | 3442822 | 3442960 |
| chr6 | 3581231 | 3581367 |
| chr6 | 3661583 | 3661711 |
| chr6 | 3844185 | 3844339 |
| chr6 | 3940475 | 3940605 |
| chr6 | 4158309 | 4158441 |
| chr6 | 4266758 | 4266884 |
| chr6 | 4465098 | 4465222 |
| chr6 | 4530038 | 4530162 |
| chr6 | 4799295 | 4799419 |
| chr6 | 4848878 | 4849022 |
| chr6 | 5049573 | 5049701 |
| chr6 | 5157352 | 5157508 |
| chr6 | 5398239 | 5398387 |
| chr6 | 5561622 | 5561744 |
| chr6 | 5660037 | 5660159 |
| chr6 | 5777069 | 5777211 |
| chr6 | 5952555 | 5952679 |
| chr6 | 6080376 | 6080518 |
| chr6 | 6194877 | 6195035 |
| chr6 | 6348750 | 6348876 |
| chr6 | 6566559 | 6566685 |
| chr6 | 6761580 | 6761726 |
| chr6 | 6870449 | 6870581 |
| chr6 | 6992700 | 6992824 |
| chr6 | 7145151 | 7145273 |
| chr6 | 7361369 | 7361523 |
| chr6 | 7445953 | 7446077 |
| chr6 | 7596756 | 7596884 |
| chr6 | 7743607 | 7743733 |
| chr6 | 7907051 | 7907179 |
| chr6 | 8073288 | 8073422 |
| chr6 | 8213798 | 8213920 |
| chr6 | 8325967 | 8326091 |
| chr6 | 8453982 | 8454134 |
| chr6 | 8690380 | 8690524 |
| chr6 | 8828643 | 8828795 |
| chr6 | 8936061 | 8936201 |
| chr6 | 9042460 | 9042584 |
| chr6 | 9213023 | 9213145 |
| chr6 | 9344715 | 9344839 |
| chr6 | 9508719 | 9508861 |
| chr6 | 9715502 | 9715626 |
| chr6 | 9843501 | 9843627 |
| chr6 | 9959217 | 9959357 |
| chr6 | 10153856 | 10154000 |
| chr6 | 10260709 | 10260847 |
| chr6 | 10463969 | 10464103 |
| chr6 | 10647965 | 10648253 |
| chr6 | 10738727 | 10738861 |
| chr6 | 10956050 | 10956174 |
| chr6 | 11082872 | 11082998 |
| chr6 | 11153774 | 11153916 |
| chr6 | 11392795 | 11392941 |
| chr6 | 11526096 | 11526220 |
| chr6 | 11701927 | 11702061 |
| chr6 | 11865453 | 11865609 |
| chr6 | 11982405 | 11982541 |
| chr6 | 12134677 | 12134801 |
| chr6 | 12307174 | 12307312 |
| chr6 | 12376941 | 12377065 |
| chr6 | 12540209 | 12540333 |
| chr6 | 12660787 | 12660953 |
| chr6 | 12908366 | 12908494 |
| chr6 | 12948306 | 12948456 |
| chr6 | 13171000 | 13171124 |
| chr6 | 13223922 | 13224044 |
| chr6 | 13514310 | 13514434 |
| chr6 | 13553961 | 13554087 |
| chr6 | 13818647 | 13818789 |
| chr6 | 13969838 | 13969968 |
| chr6 | 14004191 | 14004369 |
| chr6 | 14142409 | 14142557 |
| chr6 | 14384470 | 14384594 |
| chr6 | 14486895 | 14487021 |
| chr6 | 14689604 | 14689748 |
| chr6 | 14797017 | 14797143 |
| chr6 | 14871461 | 14871585 |
| chr6 | 15059231 | 15059387 |
| chr6 | 15184823 | 15184957 |
| chr6 | 15425423 | 15425545 |
| chr6 | 15472621 | 15472755 |
| chr6 | 15649739 | 15649881 |
| chr6 | 15912976 | 15913110 |
| chr6 | 15936489 | 15936627 |
| chr6 | 16083925 | 16084051 |
| chr6 | 16252765 | 16252887 |
| chr6 | 16508351 | 16508475 |
| chr6 | 16590072 | 16590200 |
| chr6 | 16731167 | 16731313 |
| chr6 | 16869375 | 16869501 |
| chr6 | 17091756 | 17091898 |
| chr6 | 17226007 | 17226141 |
| chr6 | 17388687 | 17388813 |
| chr6 | 17515929 | 17516077 |
| chr6 | 17709552 | 17709684 |
| chr6 | 17795147 | 17795273 |
| chr6 | 17893438 | 17893576 |
| chr6 | 18150456 | 18150592 |
| chr6 | 18262049 | 18262175 |
| chr6 | 18441584 | 18441708 |
| chr6 | 18477900 | 18478022 |
| chr6 | 18636275 | 18636407 |
| chr6 | 18805782 | 18805936 |
| chr6 | 18931358 | 18931516 |
| chr6 | 19142965 | 19143111 |
| chr6 | 19337473 | 19337603 |
| chr6 | 19416357 | 19416509 |
| chr6 | 19600459 | 19600601 |
| chr6 | 19690041 | 19690167 |
| chr6 | 19893794 | 19893930 |
| chr6 | 20031806 | 20031938 |
| chr6 | 20174374 | 20174496 |
| chr6 | 20325129 | 20325255 |
| chr6 | 20488144 | 20488266 |
| chr6 | 20614624 | 20614748 |
| chr6 | 20800082 | 20800230 |
| chr6 | 21012862 | 21012990 |
| chr6 | 21120338 | 21120486 |
| chr6 | 21205419 | 21205551 |
| chr6 | 21383328 | 21383450 |
| chr6 | 21516788 | 21516912 |
| chr6 | 21703251 | 21703375 |
| chr6 | 21844653 | 21844785 |
| chr6 | 21986658 | 21986802 |
| chr6 | 22202637 | 22202767 |
| chr6 | 22311808 | 22311936 |
| chr6 | 22391038 | 22391188 |
| chr6 | 22632611 | 22632751 |
| chr6 | 22768630 | 22768756 |
| chr6 | 22885226 | 22885348 |
| chr6 | 23025816 | 23025952 |
| chr6 | 23136782 | 23136910 |
| chr6 | 23284278 | 23284438 |
| chr6 | 23564330 | 23564454 |
| chr6 | 23639284 | 23639412 |
| chr6 | 23824670 | 23824818 |
| chr6 | 24019003 | 24019149 |
| chr6 | 24060957 | 24061113 |
| chr6 | 24286870 | 24287002 |
| chr6 | 24430974 | 24431126 |
| chr6 | 24533113 | 24533241 |
| chr6 | 24631799 | 24631927 |
| chr6 | 24820798 | 24820928 |
| chr6 | 25013992 | 25014124 |
| chr6 | 25139414 | 25139546 |
| chr6 | 25312154 | 25312306 |
| chr6 | 25494880 | 25495010 |
| chr6 | 25540847 | 25540985 |
| chr6 | 25714971 | 25715107 |
| chr6 | 25969922 | 25970046 |
| chr6 | 26083053 | 26083201 |
| chr6 | 26123449 | 26123587 |
| chr6 | 26345051 | 26345179 |
| chr6 | 26467118 | 26467260 |
| chr6 | 26663602 | 26663727 |
| chr6 | 26838931 | 26839055 |
| chr6 | 26958000 | 26958122 |
| chr6 | 26975628 | 26975750 |
| chr6 | 27164671 | 27164797 |
| chr6 | 27271303 | 27271429 |
| chr6 | 27461862 | 27462000 |
| chr6 | 27601497 | 27601643 |
| chr6 | 27709389 | 27709511 |
| chr6 | 27779416 | 27779564 |
| chr6 | 27932674 | 27932800 |
| chr6 | 28132274 | 28132406 |
| chr6 | 28270211 | 28270339 |
| chr6 | 28417174 | 28417306 |
| chr6 | 28533890 | 28534032 |
| chr6 | 28810765 | 28810887 |
| chr6 | 28892829 | 28892959 |
| chr6 | 28989959 | 28990109 |
| chr6 | 29132686 | 29132810 |
| chr6 | 29295449 | 29295573 |
| chr6 | 29446556 | 29446682 |
| chr6 | 29608509 | 29608633 |
| chr6 | 29736001 | 29736135 |
| chr6 | 29984198 | 29984340 |
| chr6 | 30119393 | 30119517 |
| chr6 | 30193477 | 30193601 |
| chr6 | 30424585 | 30424709 |
| chr6 | 30536069 | 30536217 |
| chr6 | 30717971 | 30718117 |
| chr6 | 30823029 | 30823151 |
| chr6 | 31001578 | 31001736 |
| chr6 | 31185730 | 31185866 |
| chr6 | 31354509 | 31354649 |
| chr6 | 31407559 | 31407683 |
| chr6 | 31568379 | 31568519 |
| chr6 | 31764857 | 31764987 |
| chr6 | 31941251 | 31941743 |
| chr6 | 32022634 | 32023112 |
| chr6 | 32195242 | 32195368 |
| chr6 | 32413207 | 32413343 |
| chr6 | 32441597 | 32441719 |
| chr6 | 32714684 | 32714816 |
| chr6 | 32835527 | 32835669 |
| chr6 | 32955888 | 32956020 |
| chr6 | 33032821 | 33032945 |
| chr6 | 33301624 | 33301746 |
| chr6 | 33397445 | 33397601 |
| chr6 | 33514003 | 33514159 |
| chr6 | 33689484 | 33689606 |
| chr6 | 33812955 | 33813091 |
| chr6 | 34030258 | 34030384 |
| chr6 | 34218075 | 34218199 |
| chr6 | 34343494 | 34343654 |
| chr6 | 34463920 | 34464058 |
| chr6 | 34579355 | 34579499 |
| chr6 | 34705869 | 34705999 |
| chr6 | 34944803 | 34944943 |
| chr6 | 35012755 | 35012885 |
| chr6 | 35143084 | 35143218 |
| chr6 | 35298572 | 35298696 |
| chr6 | 35567040 | 35567166 |
| chr6 | 35680068 | 35680192 |
| chr6 | 35858165 | 35858309 |
| chr6 | 35895920 | 35896068 |
| chr6 | 36022587 | 36022762 |
| chr6 | 36233410 | 36233536 |
| chr6 | 36361642 | 36361764 |
| chr6 | 36524821 | 36524963 |
| chr6 | 36668684 | 36668820 |
| chr6 | 36856665 | 36856799 |
| chr6 | 37006225 | 37006371 |
| chr6 | 37194849 | 37194995 |
| chr6 | 37289693 | 37289837 |
| chr6 | 37417468 | 37417604 |
| chr6 | 37596125 | 37596267 |
| chr6 | 37819866 | 37819992 |
| chr6 | 37849808 | 37849946 |
| chr6 | 38047102 | 38047228 |
| chr6 | 38175855 | 38175993 |
| chr6 | 38398741 | 38398889 |
| chr6 | 38443966 | 38444088 |
| chr6 | 38612460 | 38612600 |
| chr6 | 38845319 | 38845443 |
| chr6 | 39003816 | 39003962 |
| chr6 | 39168088 | 39168226 |
| chr6 | 39238198 | 39238326 |
| chr6 | 39401661 | 39401785 |
| chr6 | 39516722 | 39516866 |
| chr6 | 39732585 | 39732719 |
| chr6 | 39797294 | 39797434 |
| chr6 | 40052580 | 40052728 |
| chr6 | 40104548 | 40104678 |
| chr6 | 40279441 | 40279589 |
| chr6 | 40421392 | 40421516 |
| chr6 | 40645250 | 40645386 |
| chr6 | 40700702 | 40700860 |
| chr6 | 40871816 | 40871988 |
| chr6 | 40986569 | 40986711 |
| chr6 | 41138788 | 41139058 |
| chr6 | 41361542 | 41361672 |
| chr6 | 41502579 | 41502703 |
| chr6 | 41690785 | 41690909 |
| chr6 | 41846513 | 41846657 |
| chr6 | 41975260 | 41975398 |
| chr6 | 42039401 | 42039529 |
| chr6 | 42194826 | 42194974 |
| chr6 | 42387380 | 42387508 |
| chr6 | 42547835 | 42547965 |
| chr6 | 42671066 | 42671206 |
| chr6 | 42814237 | 42814373 |
| chr6 | 43051530 | 43051660 |
| chr6 | 43076466 | 43076632 |
| chr6 | 43298968 | 43299112 |
| chr6 | 43501976 | 43502227 |
| chr6 | 43645757 | 43645897 |
| chr6 | 43764663 | 43764831 |
| chr6 | 43932133 | 43932279 |
| chr6 | 44016117 | 44016241 |
| chr6 | 44156418 | 44156540 |
| chr6 | 44317335 | 44317487 |
| chr6 | 44474565 | 44474703 |
| chr6 | 44633542 | 44633666 |
| chr6 | 44845934 | 44846056 |
| chr6 | 45014935 | 45015063 |
| chr6 | 45141317 | 45141439 |
| chr6 | 45313597 | 45313780 |
| chr6 | 45436432 | 45436751 |
| chr6 | 45490658 | 45490785 |
| chr6 | 45677010 | 45677138 |
| chr6 | 45792086 | 45792224 |
| chr6 | 46017574 | 46017708 |
| chr6 | 46127778 | 46127910 |
| chr6 | 46239579 | 46239703 |
| chr6 | 46439843 | 46439977 |
| chr6 | 46520210 | 46520342 |
| chr6 | 46720733 | 46720859 |
| chr6 | 46876189 | 46876327 |
| chr6 | 47024742 | 47024904 |
| chr6 | 47205858 | 47205998 |
| chr6 | 47287715 | 47287855 |
| chr6 | 47480905 | 47481029 |
| chr6 | 47677851 | 47677991 |
| chr6 | 47788475 | 47788619 |
| chr6 | 47946785 | 47946909 |
| chr6 | 48031156 | 48031278 |
| chr6 | 48288965 | 48289113 |
| chr6 | 48395006 | 48395136 |
| chr6 | 48579088 | 48579234 |
| chr6 | 48627204 | 48627326 |
| chr6 | 48889161 | 48889309 |
| chr6 | 49021540 | 49021676 |
| chr6 | 49126012 | 49126150 |
| chr6 | 49290110 | 49290232 |
| chr6 | 49386526 | 49386652 |
| chr6 | 49599372 | 49599520 |
| chr6 | 49747410 | 49747532 |
| chr6 | 49823752 | 49823890 |
| chr6 | 50008548 | 50008669 |
| chr6 | 50160039 | 50160161 |
| chr6 | 50310237 | 50310365 |
| chr6 | 50525581 | 50525705 |
| chr6 | 50576608 | 50576730 |
| chr6 | 50739028 | 50739154 |
| chr6 | 50878149 | 50878275 |
| chr6 | 51140564 | 51140730 |
| chr6 | 51265518 | 51265644 |
| chr6 | 51356989 | 51357111 |
| chr6 | 51589043 | 51589171 |
| chr6 | 51656465 | 51656599 |
| chr6 | 51863584 | 51863726 |
| chr6 | 52036242 | 52036386 |
| chr6 | 52072276 | 52072420 |
| chr6 | 52240884 | 52241046 |
| chr6 | 52447196 | 52447328 |
| chr6 | 52544812 | 52544936 |
| chr6 | 52713844 | 52713990 |
| chr6 | 52840147 | 52840269 |
| chr6 | 53064245 | 53064385 |
| chr6 | 53210082 | 53210216 |
| chr6 | 53298433 | 53298561 |
| chr6 | 53470805 | 53470943 |
| chr6 | 53651097 | 53651223 |
| chr6 | 53837913 | 53838065 |
| chr6 | 53938936 | 53939060 |
| chr6 | 54025777 | 54025899 |
| chr6 | 54184154 | 54184296 |
| chr6 | 54452761 | 54452885 |
| chr6 | 54509811 | 54509939 |
| chr6 | 54682243 | 54682367 |
| chr6 | 54917412 | 54917536 |
| chr6 | 55017841 | 55017983 |
| chr6 | 55203975 | 55204099 |
| chr6 | 55243111 | 55243237 |
| chr6 | 55470604 | 55470756 |
| chr6 | 55626028 | 55626150 |
| chr6 | 55778108 | 55778238 |
| chr6 | 55892470 | 55892606 |
| chr6 | 56105897 | 56106025 |
| chr6 | 56268448 | 56268572 |
| chr6 | 56326687 | 56326813 |
| chr6 | 56551717 | 56551860 |
| chr6 | 56570613 | 56570739 |
| chr6 | 56822929 | 56823055 |
| chr6 | 56933112 | 56933244 |
| chr6 | 57112040 | 57112178 |
| chr6 | 57313091 | 57313363 |
| chr6 | 57337616 | 57337795 |
| chr6 | 57534345 | 57534610 |
| chr6 | 57705933 | 57706079 |
| chr6 | 57719309 | 57719461 |
| chr6 | 57810217 | 57810341 |
| chr6 | 58014648 | 58014792 |
| chr6 | 58178594 | 58178744 |
| chr6 | 58238211 | 58238335 |
| chr6 | 58312992 | 58313138 |
| chr6 | 58398676 | 58398798 |
| chr6 | 58417039 | 58417163 |
| chr6 | 58616597 | 58616721 |
| chr6 | 61969009 | 61969135 |
| chr6 | 61996327 | 61996451 |
| chr6 | 62248218 | 62248348 |
| chr6 | 62411805 | 62411939 |
| chr6 | 62427759 | 62427885 |
| chr6 | 62678225 | 62678359 |
| chr6 | 62851635 | 62851759 |
| chr6 | 62971771 | 62971899 |
| chr6 | 63031527 | 63031663 |
| chr6 | 63182559 | 63182683 |
| chr6 | 63414918 | 63415046 |
| chr6 | 63610737 | 63610875 |
| chr6 | 63698397 | 63698535 |
| chr6 | 63803152 | 63803276 |
| chr6 | 63979740 | 63979882 |
| chr6 | 64101191 | 64101445 |
| chr6 | 64330499 | 64330635 |
| chr6 | 64469993 | 64470131 |
| chr6 | 64595883 | 64596029 |
| chr6 | 64773009 | 64773139 |
| chr6 | 64858279 | 64858417 |
| chr6 | 65087637 | 65087761 |
| chr6 | 65236767 | 65236899 |
| chr6 | 65373123 | 65373251 |
| chr6 | 65506874 | 65506998 |
| chr6 | 65591484 | 65591612 |
| chr6 | 65823808 | 65823932 |
| chr6 | 65979476 | 65979602 |
| chr6 | 66074515 | 66074643 |
| chr6 | 66268670 | 66268794 |
| chr6 | 66424301 | 66424431 |
| chr6 | 66604937 | 66605075 |
| chr6 | 66734220 | 66734374 |
| chr6 | 66916199 | 66916329 |
| chr6 | 67018192 | 67018318 |
| chr6 | 67139790 | 67139914 |
| chr6 | 67249067 | 67249189 |
| chr6 | 67405561 | 67405727 |
| chr6 | 67605033 | 67605165 |
| chr6 | 67674328 | 67674450 |
| chr6 | 67825272 | 67825396 |
| chr6 | 68108860 | 68109020 |
| chr6 | 68217999 | 68218123 |
| chr6 | 68352576 | 68352700 |
| chr6 | 68460899 | 68461021 |
| chr6 | 68672966 | 68673114 |
| chr6 | 68764612 | 68764746 |
| chr6 | 68882271 | 68882407 |
| chr6 | 69085763 | 69085919 |
| chr6 | 69200430 | 69200554 |
| chr6 | 69335231 | 69335389 |
| chr6 | 69545954 | 69546084 |
| chr6 | 69641934 | 69642058 |
| chr6 | 69914656 | 69914792 |
| chr6 | 69984972 | 69985102 |
| chr6 | 70145077 | 70145203 |
| chr6 | 70287555 | 70287677 |
| chr6 | 70482931 | 70483061 |
| chr6 | 70569075 | 70569211 |
| chr6 | 70700284 | 70700426 |
| chr6 | 70943501 | 70943625 |
| chr6 | 71047402 | 71047524 |
| chr6 | 71249676 | 71249800 |
| chr6 | 71333102 | 71333226 |
| chr6 | 71551998 | 71552122 |
| chr6 | 71632018 | 71632144 |
| chr6 | 71815266 | 71815402 |
| chr6 | 71916340 | 71916468 |
| chr6 | 72159855 | 72159979 |
| chr6 | 72300460 | 72300582 |
| chr6 | 72349186 | 72349316 |
| chr6 | 72551541 | 72551679 |
| chr6 | 72624548 | 72624682 |
| chr6 | 72836688 | 72836832 |
| chr6 | 72991192 | 72991315 |
| chr6 | 73161894 | 73162020 |
| chr6 | 73337641 | 73337765 |
| chr6 | 73498049 | 73498223 |
| chr6 | 73535893 | 73536015 |
| chr6 | 73683687 | 73683829 |
| chr6 | 73899268 | 73899390 |
| chr6 | 74051132 | 74051256 |
| chr6 | 74200644 | 74200768 |
| chr6 | 74381740 | 74381864 |
| chr6 | 74506252 | 74506376 |
| chr6 | 74598009 | 74598141 |
| chr6 | 74779023 | 74779175 |
| chr6 | 74883381 | 74883523 |
| chr6 | 75061707 | 75061845 |
| chr6 | 75275009 | 75275155 |
| chr6 | 75349655 | 75349795 |
| chr6 | 75489957 | 75490093 |
| chr6 | 75626840 | 75627037 |
| chr6 | 75906138 | 75906262 |
| chr6 | 75997068 | 75997210 |
| chr6 | 76114266 | 76114402 |
| chr6 | 76366640 | 76366772 |
| chr6 | 76416619 | 76416743 |
| chr6 | 76574055 | 76574195 |
| chr6 | 76786478 | 76786604 |
| chr6 | 76882728 | 76882870 |
| chr6 | 77032470 | 77032592 |
| chr6 | 77177014 | 77177152 |
| chr6 | 77351019 | 77351177 |
| chr6 | 77513998 | 77514146 |
| chr6 | 77602447 | 77602583 |
| chr6 | 77852420 | 77852542 |
| chr6 | 78003183 | 78003305 |
| chr6 | 78059295 | 78059439 |
| chr6 | 78178745 | 78178871 |
| chr6 | 78391995 | 78392117 |
| chr6 | 78523717 | 78523841 |
| chr6 | 78626157 | 78626287 |
| chr6 | 78858578 | 78858702 |
| chr6 | 78983949 | 78984071 |
| chr6 | 79214207 | 79214337 |
| chr6 | 79335921 | 79336051 |
| chr6 | 79480404 | 79480546 |
| chr6 | 79613727 | 79613849 |
| chr6 | 79789934 | 79790058 |
| chr6 | 79922190 | 79922312 |
| chr6 | 80043996 | 80044122 |
| chr6 | 80225623 | 80225751 |
| chr6 | 80385623 | 80385749 |
| chr6 | 80530268 | 80530400 |
| chr6 | 80584318 | 80584440 |
| chr6 | 80826245 | 80826369 |
| chr6 | 80978973 | 80979103 |
| chr6 | 81163355 | 81163489 |
| chr6 | 81233458 | 81233584 |
| chr6 | 81436097 | 81436243 |
| chr6 | 81543101 | 81543259 |
| chr6 | 81632411 | 81632547 |
| chr6 | 81846985 | 81847125 |
| chr6 | 82068357 | 82068491 |
| chr6 | 82159446 | 82159578 |
| chr6 | 82286279 | 82286401 |
| chr6 | 82503143 | 82503297 |
| chr6 | 82660322 | 82660460 |
| chr6 | 82679178 | 82679318 |
| chr6 | 82849245 | 82849391 |
| chr6 | 83063760 | 83063888 |
| chr6 | 83135170 | 83135298 |
| chr6 | 83403260 | 83403386 |
| chr6 | 83539387 | 83539547 |
| chr6 | 83713967 | 83714115 |
| chr6 | 83804117 | 83804241 |
| chr6 | 83911629 | 83911757 |
| chr6 | 84066677 | 84066823 |
| chr6 | 84314341 | 84314475 |
| chr6 | 84354057 | 84354183 |
| chr6 | 84591830 | 84592076 |
| chr6 | 84702731 | 84702981 |
| chr6 | 84867862 | 84868032 |
| chr6 | 85043388 | 85043526 |
| chr6 | 85113231 | 85113379 |
| chr6 | 85235996 | 85236126 |
| chr6 | 85454325 | 85454453 |
| chr6 | 85653724 | 85653856 |
| chr6 | 85770392 | 85770544 |
| chr6 | 85881323 | 85881461 |
| chr6 | 86067471 | 86067595 |
| chr6 | 86223226 | 86223356 |
| chr6 | 86308641 | 86308765 |
| chr6 | 86566429 | 86566577 |
| chr6 | 86658240 | 86658362 |
| chr6 | 86729601 | 86729723 |
| chr6 | 87005951 | 87006097 |
| chr6 | 87093260 | 87093382 |
| chr6 | 87265515 | 87265637 |
| chr6 | 87449526 | 87449648 |
| chr6 | 87533485 | 87533635 |
| chr6 | 87761823 | 87761949 |
| chr6 | 87809316 | 87809466 |
| chr6 | 87978740 | 87978868 |
| chr6 | 88146511 | 88146649 |
| chr6 | 88230650 | 88230782 |
| chr6 | 88465497 | 88465641 |
| chr6 | 88580574 | 88580712 |
| chr6 | 88676337 | 88676481 |
| chr6 | 88896872 | 88897026 |
| chr6 | 89060187 | 89060343 |
| chr6 | 89197234 | 89197360 |
| chr6 | 89396943 | 89397089 |
| chr6 | 89420250 | 89420376 |
| chr6 | 89586963 | 89587113 |
| chr6 | 89855371 | 89855493 |
| chr6 | 89882779 | 89882931 |
| chr6 | 90140474 | 90140612 |
| chr6 | 90246647 | 90246801 |
| chr6 | 90446495 | 90446782 |
| chr6 | 90552119 | 90552267 |
| chr6 | 90745258 | 90745408 |
| chr6 | 90811805 | 90811947 |
| chr6 | 90981567 | 90981693 |
| chr6 | 91140374 | 91140506 |
| chr6 | 91268305 | 91268429 |
| chr6 | 91427299 | 91427427 |
| chr6 | 91521163 | 91521295 |
| chr6 | 91816497 | 91816627 |
| chr6 | 91891583 | 91891723 |
| chr6 | 92018996 | 92019134 |
| chr6 | 92204443 | 92204581 |
| chr6 | 92344163 | 92344287 |
| chr6 | 92458802 | 92458950 |
| chr6 | 92639014 | 92639158 |
| chr6 | 92798514 | 92798638 |
| chr6 | 93009647 | 93009805 |
| chr6 | 93062089 | 93062213 |
| chr6 | 93299407 | 93299537 |
| chr6 | 93349437 | 93349573 |
| chr6 | 93602524 | 93602646 |
| chr6 | 93741362 | 93741484 |
| chr6 | 93788765 | 93788909 |
| chr6 | 93963361 | 93963489 |
| chr6 | 94187676 | 94187830 |
| chr6 | 94260966 | 94261112 |
| chr6 | 94468434 | 94468558 |
| chr6 | 94662778 | 94662908 |
| chr6 | 94694045 | 94694191 |
| chr6 | 94844156 | 94844280 |
| chr6 | 94993974 | 94994112 |
| chr6 | 95212647 | 95212771 |
| chr6 | 95377304 | 95377458 |
| chr6 | 95540524 | 95540650 |
| chr6 | 95610716 | 95610837 |
| chr6 | 95851613 | 95851741 |
| chr6 | 96016744 | 96016884 |
| chr6 | 96116973 | 96117095 |
| chr6 | 96239850 | 96239976 |
| chr6 | 96348255 | 96348395 |
| chr6 | 96534583 | 96534761 |
| chr6 | 96658616 | 96658748 |
| chr6 | 96858373 | 96858515 |
| chr6 | 97006493 | 97006615 |
| chr6 | 97186510 | 97186652 |
| chr6 | 97224734 | 97224870 |
| chr6 | 97371763 | 97371893 |
| chr6 | 97616633 | 97616759 |
| chr6 | 97807318 | 97807448 |
| chr6 | 97922118 | 97922254 |
| chr6 | 98097321 | 98097443 |
| chr6 | 98181533 | 98181655 |
| chr6 | 98378432 | 98378560 |
| chr6 | 98553570 | 98553698 |
| chr6 | 98704416 | 98704566 |
| chr6 | 98810456 | 98810600 |
| chr6 | 99019357 | 99019479 |
| chr6 | 99052008 | 99052146 |
| chr6 | 99292100 | 99292222 |
| chr6 | 99375550 | 99375692 |
| chr6 | 99583683 | 99583821 |
| chr6 | 99728356 | 99728478 |
| chr6 | 99793039 | 99793187 |
| chr6 | 100030995 | 100031123 |
| chr6 | 100093905 | 100094033 |
| chr6 | 100242993 | 100243137 |
| chr6 | 100473312 | 100473466 |
| chr6 | 100564148 | 100564278 |
| chr6 | 100704086 | 100704216 |
| chr6 | 100864866 | 100864992 |
| chr6 | 100988887 | 100989009 |
| chr6 | 101227538 | 101227668 |
| chr6 | 101362789 | 101362913 |
| chr6 | 101540785 | 101540927 |
| chr6 | 101588932 | 101589078 |
| chr6 | 101765534 | 101765664 |
| chr6 | 101871211 | 101871335 |
| chr6 | 102065108 | 102065248 |
| chr6 | 102270949 | 102271079 |
| chr6 | 102352514 | 102352642 |
| chr6 | 102474503 | 102474627 |
| chr6 | 102706987 | 102707129 |
| chr6 | 102894021 | 102894147 |
| chr6 | 102931820 | 102931942 |
| chr6 | 103165317 | 103165465 |
| chr6 | 103229306 | 103229428 |
| chr6 | 103515678 | 103515802 |
| chr6 | 103564268 | 103564390 |
| chr6 | 103685089 | 103685239 |
| chr6 | 103897871 | 103898013 |
| chr6 | 104000608 | 104000744 |
| chr6 | 104214190 | 104214314 |
| chr6 | 104321648 | 104321772 |
| chr6 | 104439915 | 104440037 |
| chr6 | 104582489 | 104582631 |
| chr6 | 104815083 | 104815223 |
| chr6 | 104905887 | 104906035 |
| chr6 | 105137036 | 105137174 |
| chr6 | 105223601 | 105223733 |
| chr6 | 105389871 | 105389995 |
| chr6 | 105546400 | 105546528 |
| chr6 | 105652099 | 105652245 |
| chr6 | 105799373 | 105799507 |
| chr6 | 106044307 | 106044435 |
| chr6 | 106114847 | 106114969 |
| chr6 | 106358775 | 106358921 |
| chr6 | 106427434 | 106427578 |
| chr6 | 106564394 | 106564518 |
| chr6 | 106777986 | 106778124 |
| chr6 | 106913880 | 106914002 |
| chr6 | 107051183 | 107051305 |
| chr6 | 107158569 | 107158719 |
| chr6 | 107393130 | 107393264 |
| chr6 | 107446450 | 107446588 |
| chr6 | 107581116 | 107581244 |
| chr6 | 107817703 | 107817825 |
| chr6 | 107921135 | 107921285 |
| chr6 | 108041587 | 108041709 |
| chr6 | 108261235 | 108261363 |
| chr6 | 108322416 | 108322538 |
| chr6 | 108529001 | 108529143 |
| chr6 | 108632221 | 108632482 |
| chr6 | 108841650 | 108841796 |
| chr6 | 109049536 | 109049658 |
| chr6 | 109105235 | 109105357 |
| chr6 | 109237371 | 109237495 |
| chr6 | 109376190 | 109376434 |
| chr6 | 109640233 | 109640375 |
| chr6 | 109714994 | 109715118 |
| chr6 | 109839024 | 109839170 |
| chr6 | 110049952 | 110050078 |
| chr6 | 110124468 | 110124608 |
| chr6 | 110355740 | 110355884 |
| chr6 | 110548530 | 110548676 |
| chr6 | 110589509 | 110589631 |
| chr6 | 110751485 | 110751607 |
| chr6 | 110878347 | 110878515 |
| chr6 | 111100639 | 111100765 |
| chr6 | 111282668 | 111282800 |
| chr6 | 111415560 | 111415692 |
| chr6 | 111515153 | 111515291 |
| chr6 | 111676475 | 111676601 |
| chr6 | 111850555 | 111850681 |
| chr6 | 112014875 | 112014999 |
| chr6 | 112106236 | 112106376 |
| chr6 | 112332333 | 112332469 |
| chr6 | 112462986 | 112463144 |
| chr6 | 112664311 | 112664437 |
| chr6 | 112679252 | 112679392 |
| chr6 | 112933250 | 112933376 |
| chr6 | 113040994 | 113041118 |
| chr6 | 113132182 | 113132308 |
| chr6 | 113405188 | 113405362 |
| chr6 | 113532508 | 113532632 |
| chr6 | 113605744 | 113605882 |
| chr6 | 113759023 | 113759161 |
| chr6 | 113984418 | 113984556 |
| chr6 | 114045615 | 114045737 |
| chr6 | 114303010 | 114303134 |
| chr6 | 114386400 | 114386536 |
| chr6 | 114581669 | 114581793 |
| chr6 | 114634383 | 114634509 |
| chr6 | 114897170 | 114897294 |
| chr6 | 114943490 | 114943618 |
| chr6 | 115135093 | 115135235 |
| chr6 | 115326841 | 115326979 |
| chr6 | 115451590 | 115451732 |
| chr6 | 115585213 | 115585353 |
| chr6 | 115765064 | 115765188 |
| chr6 | 115906225 | 115906371 |
| chr6 | 116105914 | 116106038 |
| chr6 | 116253721 | 116253845 |
| chr6 | 116373471 | 116373619 |
| chr6 | 116475469 | 116475595 |
| chr6 | 116626055 | 116626185 |
| chr6 | 116796686 | 116796810 |
| chr6 | 116999953 | 117000101 |
| chr6 | 117157688 | 117157836 |
| chr6 | 117217890 | 117218020 |
| chr6 | 117353036 | 117353164 |
| chr6 | 117560801 | 117560949 |
| chr6 | 117721979 | 117722149 |
| chr6 | 117806375 | 117806511 |
| chr6 | 117965674 | 117965798 |
| chr6 | 118109734 | 118109872 |
| chr6 | 118275870 | 118276024 |
| chr6 | 118405708 | 118405860 |
| chr6 | 118627447 | 118627585 |
| chr6 | 118733129 | 118733253 |
| chr6 | 118826004 | 118826142 |
| chr6 | 118999334 | 118999460 |
| chr6 | 119150769 | 119150893 |
| chr6 | 119374661 | 119374807 |
| chr6 | 119519016 | 119519158 |
| chr6 | 119593944 | 119594100 |
| chr6 | 119859980 | 119860136 |
| chr6 | 119998248 | 119998382 |
| chr6 | 120083055 | 120083185 |
| chr6 | 120196654 | 120196778 |
| chr6 | 120460775 | 120460911 |
| chr6 | 120535553 | 120535679 |
| chr6 | 120743006 | 120743128 |
| chr6 | 120874819 | 120874941 |
| chr6 | 121040311 | 121040447 |
| chr6 | 121198564 | 121198708 |
| chr6 | 121343724 | 121343846 |
| chr6 | 121414688 | 121414812 |
| chr6 | 121651536 | 121651666 |
| chr6 | 121683648 | 121683774 |
| chr6 | 121884893 | 121885017 |
| chr6 | 122032893 | 122033021 |
| chr6 | 122189086 | 122189208 |
| chr6 | 122399807 | 122399931 |
| chr6 | 122455742 | 122455886 |
| chr6 | 122596450 | 122596574 |
| chr6 | 122765910 | 122766034 |
| chr6 | 122972821 | 122972953 |
| chr6 | 123080621 | 123080755 |
| chr6 | 123305883 | 123306017 |
| chr6 | 123438395 | 123438529 |
| chr6 | 123493088 | 123493212 |
| chr6 | 123722089 | 123722217 |
| chr6 | 123902489 | 123902617 |
| chr6 | 123970236 | 123970364 |
| chr6 | 124216964 | 124217104 |
| chr6 | 124310190 | 124310320 |
| chr6 | 124421032 | 124421156 |
| chr6 | 124651270 | 124651410 |
| chr6 | 124715123 | 124715253 |
| chr6 | 124880127 | 124880251 |
| chr6 | 124990340 | 124990464 |
| chr6 | 125261795 | 125261917 |
| chr6 | 125388109 | 125388231 |
| chr6 | 125508359 | 125508501 |
| chr6 | 125623851 | 125623989 |
| chr6 | 125859119 | 125859241 |
| chr6 | 125921590 | 125921714 |
| chr6 | 126090225 | 126090347 |
| chr6 | 126284139 | 126284331 |
| chr6 | 126372461 | 126372713 |
| chr6 | 126481349 | 126481489 |
| chr6 | 126753369 | 126753527 |
| chr6 | 126834749 | 126835012 |
| chr6 | 126923721 | 126923961 |
| chr6 | 127201774 | 127201920 |
| chr6 | 127263474 | 127263620 |
| chr6 | 127392923 | 127393051 |
| chr6 | 127663038 | 127663172 |
| chr6 | 127772067 | 127772209 |
| chr6 | 127895075 | 127895199 |
| chr6 | 128056240 | 128056388 |
| chr6 | 128148729 | 128148881 |
| chr6 | 128311505 | 128311651 |
| chr6 | 128431490 | 128431731 |
| chr6 | 128584525 | 128584665 |
| chr6 | 128847214 | 128847462 |
| chr6 | 128988769 | 128988893 |
| chr6 | 129149417 | 129149541 |
| chr6 | 129257966 | 129258147 |
| chr6 | 129342705 | 129342948 |
| chr6 | 129573957 | 129574089 |
| chr6 | 129621064 | 129621192 |
| chr6 | 129866189 | 129866325 |
| chr6 | 129989243 | 129989389 |
| chr6 | 130155662 | 130155790 |
| chr6 | 130368446 | 130368590 |
| chr6 | 130441422 | 130441548 |
| chr6 | 130649514 | 130649668 |
| chr6 | 130816446 | 130816602 |
| chr6 | 130889981 | 130890117 |
| chr6 | 131094031 | 131094155 |
| chr6 | 131159686 | 131159824 |
| chr6 | 131419306 | 131419430 |
| chr6 | 131548980 | 131549110 |
| chr6 | 131673155 | 131673281 |
| chr6 | 131721129 | 131721253 |
| chr6 | 132011912 | 132012052 |
| chr6 | 132140151 | 132140281 |
| chr6 | 132303624 | 132303762 |
| chr6 | 132380115 | 132380237 |
| chr6 | 132611006 | 132611175 |
| chr6 | 132654787 | 132654911 |
| chr6 | 132854593 | 132854721 |
| chr6 | 132964782 | 132964908 |
| chr6 | 133085379 | 133085509 |
| chr6 | 133340851 | 133340975 |
| chr6 | 133480125 | 133480261 |
| chr6 | 133613843 | 133613971 |
| chr6 | 133779997 | 133780137 |
| chr6 | 133969923 | 133970045 |
| chr6 | 134073156 | 134073292 |
| chr6 | 134147075 | 134147205 |
| chr6 | 134288032 | 134288186 |
| chr6 | 134519360 | 134519490 |
| chr6 | 134685625 | 134685749 |
| chr6 | 134758444 | 134758568 |
| chr6 | 135013880 | 135014002 |
| chr6 | 135101098 | 135101222 |
| chr6 | 135185788 | 135185930 |
| chr6 | 135451490 | 135451614 |
| chr6 | 135617639 | 135617832 |
| chr6 | 135725431 | 135725573 |
| chr6 | 135812412 | 135812656 |
| chr6 | 136021056 | 136021180 |
| chr6 | 136151157 | 136151281 |
| chr6 | 136295820 | 136295942 |
| chr6 | 136474704 | 136474834 |
| chr6 | 136572225 | 136572351 |
| chr6 | 136713663 | 136713813 |
| chr6 | 136952882 | 136953016 |
| chr6 | 137085220 | 137085344 |
| chr6 | 137134449 | 137134601 |
| chr6 | 137282043 | 137282167 |
| chr6 | 137530993 | 137531117 |
| chr6 | 137578078 | 137578256 |
| chr6 | 137726562 | 137726684 |
| chr6 | 137933405 | 137933531 |
| chr6 | 138146425 | 138146557 |
| chr6 | 138222420 | 138222564 |
| chr6 | 138406246 | 138406388 |
| chr6 | 138496604 | 138496728 |
| chr6 | 138734380 | 138734504 |
| chr6 | 138865270 | 138865410 |
| chr6 | 139069585 | 139069709 |
| chr6 | 139141099 | 139141225 |
| chr6 | 139227499 | 139227623 |
| chr6 | 139437493 | 139437625 |
| chr6 | 139663811 | 139663935 |
| chr6 | 139761324 | 139761446 |
| chr6 | 139854588 | 139854736 |
| chr6 | 140036312 | 140036436 |
| chr6 | 140256623 | 140256753 |
| chr6 | 140369697 | 140369847 |
| chr6 | 140462935 | 140463065 |
| chr6 | 140646415 | 140646547 |
| chr6 | 140757024 | 140757150 |
| chr6 | 140942484 | 140942638 |
| chr6 | 141063161 | 141063283 |
| chr6 | 141200604 | 141200728 |
| chr6 | 141328775 | 141328899 |
| chr6 | 141472563 | 141472695 |
| chr6 | 141664282 | 141664414 |
| chr6 | 141916464 | 141916602 |
| chr6 | 141937370 | 141937506 |
| chr6 | 142182543 | 142182665 |
| chr6 | 142352368 | 142352520 |
| chr6 | 142421822 | 142421952 |
| chr6 | 142648169 | 142648297 |
| chr6 | 142679479 | 142679724 |
| chr6 | 142870829 | 142870955 |
| chr6 | 142970219 | 142970345 |
| chr6 | 143228845 | 143228985 |
| chr6 | 143340604 | 143340746 |
| chr6 | 143561993 | 143562119 |
| chr6 | 143600940 | 143601062 |
| chr6 | 143741255 | 143741389 |
| chr6 | 143993173 | 143993313 |
| chr6 | 144022169 | 144022311 |
| chr6 | 144287136 | 144287266 |
| chr6 | 144358229 | 144358359 |
| chr6 | 144569939 | 144570063 |
| chr6 | 144671719 | 144671859 |
| chr6 | 144862908 | 144863032 |
| chr6 | 145062567 | 145062689 |
| chr6 | 145176085 | 145176225 |
| chr6 | 145253801 | 145253925 |
| chr6 | 145485958 | 145486082 |
| chr6 | 145560371 | 145560499 |
| chr6 | 145773892 | 145774018 |
| chr6 | 145911250 | 145911374 |
| chr6 | 146118903 | 146119031 |
| chr6 | 146162771 | 146162903 |
| chr6 | 146329593 | 146329749 |
| chr6 | 146537635 | 146537791 |
| chr6 | 146620589 | 146620715 |
| chr6 | 146782416 | 146782544 |
| chr6 | 146953876 | 146954002 |
| chr6 | 147070453 | 147070593 |
| chr6 | 147302302 | 147302460 |
| chr6 | 147353514 | 147353638 |
| chr6 | 147549548 | 147549674 |
| chr6 | 147767157 | 147767293 |
| chr6 | 147915378 | 147915522 |
| chr6 | 147981401 | 147981525 |
| chr6 | 148109088 | 148109210 |
| chr6 | 148229581 | 148229717 |
| chr6 | 148391739 | 148391863 |
| chr6 | 148565381 | 148565535 |
| chr6 | 148779530 | 148779686 |
| chr6 | 148919859 | 148919981 |
| chr6 | 149049005 | 149049129 |
| chr6 | 149230413 | 149230561 |
| chr6 | 149301049 | 149301193 |
| chr6 | 149429380 | 149429534 |
| chr6 | 149607614 | 149607742 |
| chr6 | 149735384 | 149735514 |
| chr6 | 149881769 | 149881893 |
| chr6 | 150139951 | 150140091 |
| chr6 | 150204864 | 150205006 |
| chr6 | 150369255 | 150369381 |
| chr6 | 150499827 | 150499977 |
| chr6 | 150676448 | 150676590 |
| chr6 | 150848292 | 150848420 |
| chr6 | 150986272 | 150986406 |
| chr6 | 151158901 | 151159025 |
| chr6 | 151317148 | 151317274 |
| chr6 | 151378265 | 151378399 |
| chr6 | 151596412 | 151596534 |
| chr6 | 151815929 | 151816059 |
| chr6 | 151919896 | 151920024 |
| chr6 | 152016283 | 152016421 |
| chr6 | 152157070 | 152157194 |
| chr6 | 152355832 | 152355982 |
| chr6 | 152428658 | 152428786 |
| chr6 | 152674035 | 152674163 |
| chr6 | 152836811 | 152836955 |
| chr6 | 152973247 | 152973373 |
| chr6 | 153127309 | 153127433 |
| chr6 | 153171329 | 153171453 |
| chr6 | 153342885 | 153343033 |
| chr6 | 153613951 | 153614077 |
| chr6 | 153743592 | 153743728 |
| chr6 | 153777015 | 153777143 |
| chr6 | 153977468 | 153977608 |
| chr6 | 154179035 | 154179165 |
| chr6 | 154350065 | 154350328 |
| chr6 | 154449788 | 154449928 |
| chr6 | 154530918 | 154531052 |
| chr6 | 154677639 | 154677799 |
| chr6 | 154821104 | 154821246 |
| chr6 | 155002612 | 155002736 |
| chr6 | 155170893 | 155171019 |
| chr6 | 155284406 | 155284558 |
| chr6 | 155466462 | 155466588 |
| chr6 | 155620937 | 155621069 |
| chr6 | 155755890 | 155756016 |
| chr6 | 156012268 | 156012428 |
| chr6 | 156119782 | 156119928 |
| chr6 | 156255013 | 156255139 |
| chr6 | 156386322 | 156386454 |
| chr6 | 156479921 | 156480063 |
| chr6 | 156769705 | 156769847 |
| chr6 | 156894078 | 156894206 |
| chr6 | 157041930 | 157042084 |
| chr6 | 157215983 | 157216109 |
| chr6 | 157255676 | 157255800 |
| chr6 | 157499274 | 157499396 |
| chr6 | 157615848 | 157615969 |
| chr6 | 157810153 | 157810283 |
| chr6 | 157943373 | 157943495 |
| chr6 | 158099231 | 158099355 |
| chr6 | 158230212 | 158230342 |
| chr6 | 158367977 | 158368121 |
| chr6 | 158541123 | 158541253 |
| chr6 | 158666729 | 158666861 |
| chr6 | 158761745 | 158761923 |
| chr6 | 159010305 | 159010427 |
| chr6 | 159115695 | 159115819 |
| chr6 | 159282286 | 159282426 |
| chr6 | 159425800 | 159425936 |
| chr6 | 159619536 | 159619662 |
| chr6 | 159725525 | 159725663 |
| chr6 | 159810888 | 159811014 |
| chr6 | 159944916 | 159945040 |
| chr6 | 160141502 | 160141632 |
| chr6 | 160248028 | 160248174 |
| chr6 | 160428087 | 160428211 |
| chr6 | 160622466 | 160622590 |
| chr6 | 160706584 | 160706726 |
| chr6 | 160890752 | 160890896 |
| chr6 | 161026495 | 161026789 |
| chr6 | 161252621 | 161252771 |
| chr6 | 161345087 | 161345223 |
| chr6 | 161478408 | 161478532 |
| chr6 | 161623125 | 161623265 |
| chr6 | 161869978 | 161870102 |
| chr6 | 161928819 | 161928959 |
| chr6 | 162128325 | 162128451 |
| chr6 | 162181871 | 162181993 |
| chr6 | 162344676 | 162344798 |
| chr6 | 162490966 | 162491130 |
| chr6 | 162652660 | 162652824 |
| chr6 | 162829593 | 162829721 |
| chr6 | 162948785 | 162948913 |
| chr6 | 163220047 | 163220171 |
| chr6 | 163362306 | 163362428 |
| chr6 | 163516995 | 163517119 |
| chr6 | 163631568 | 163631712 |
| chr6 | 163693571 | 163693701 |
| chr6 | 163858745 | 163858867 |
| chr6 | 164099832 | 164099954 |
| chr6 | 164236194 | 164236340 |
| chr6 | 164324142 | 164324266 |
| chr6 | 164420764 | 164420916 |
| chr6 | 164704245 | 164704373 |
| chr6 | 164752785 | 164752917 |
| chr6 | 164992069 | 164992205 |
| chr6 | 165093101 | 165093223 |
| chr6 | 165209242 | 165209368 |
| chr6 | 165350134 | 165350260 |
| chr6 | 165507433 | 165507557 |
| chr6 | 165767786 | 165767910 |
| chr6 | 165908213 | 165908347 |
| chr6 | 165985002 | 165985136 |
| chr6 | 166125219 | 166125357 |
| chr6 | 166241781 | 166241909 |
| chr6 | 166425736 | 166425882 |
| chr6 | 166594388 | 166594524 |
| chr6 | 166760214 | 166760338 |
| chr6 | 166830616 | 166830748 |
| chr6 | 167064329 | 167064451 |
| chr6 | 167229581 | 167229705 |
| chr6 | 167325972 | 167326096 |
| chr6 | 167533002 | 167533142 |
| chr6 | 167647820 | 167647942 |
| chr6 | 167727442 | 167727598 |
| chr6 | 167932405 | 167932525 |
| chr6 | 168042456 | 168042580 |
| chr6 | 168224202 | 168224344 |
| chr6 | 168430821 | 168430971 |
| chr6 | 168595835 | 168595971 |
| chr6 | 168753735 | 168753863 |
| chr6 | 168875272 | 168875426 |
| chr6 | 168991286 | 168991410 |
| chr6 | 169146773 | 169146911 |
| chr6 | 169276994 | 169277134 |
| chr6 | 169465009 | 169465161 |
| chr6 | 169643956 | 169644112 |
| chr6 | 169733208 | 169733332 |
| chr6 | 169937081 | 169937215 |
| chr6 | 170113198 | 170113332 |
| chr6 | 170187800 | 170187946 |
| chr6 | 170332971 | 170333162 |
| chr6 | 170471326 | 170471464 |
| chr6 | 170693994 | 170694118 |
| chr6 | 170867167 | 170867291 |
| chr6 | 170871511 | 170871661 |
| chr7 | 10663 | 10785 |
| chr7 | 14786 | 14916 |
| chr7 | 198323 | 198449 |
| chr7 | 290033 | 290171 |
| chr7 | 436746 | 436896 |
| chr7 | 569375 | 569505 |
| chr7 | 762492 | 762614 |
| chr7 | 942956 | 943084 |
| chr7 | 1066259 | 1066385 |
| chr7 | 1232105 | 1232237 |
| chr7 | 1331741 | 1331877 |
| chr7 | 1460880 | 1461004 |
| chr7 | 1669257 | 1669383 |
| chr7 | 1841681 | 1841803 |
| chr7 | 1983883 | 1984007 |
| chr7 | 2150303 | 2150427 |
| chr7 | 2272896 | 2273020 |
| chr7 | 2414671 | 2414795 |
| chr7 | 2505491 | 2505625 |
| chr7 | 2729697 | 2729823 |
| chr7 | 2894378 | 2894504 |
| chr7 | 3007294 | 3007432 |
| chr7 | 3146997 | 3147133 |
| chr7 | 3264468 | 3264604 |
| chr7 | 3424221 | 3424359 |
| chr7 | 3648407 | 3648535 |
| chr7 | 3754284 | 3754434 |
| chr7 | 3910621 | 3910745 |
| chr7 | 3960434 | 3960570 |
| chr7 | 4111565 | 4111689 |
| chr7 | 4332192 | 4332332 |
| chr7 | 4505270 | 4505398 |
| chr7 | 4614271 | 4614397 |
| chr7 | 4788800 | 4788924 |
| chr7 | 4947309 | 4947441 |
| chr7 | 5108586 | 5108710 |
| chr7 | 5248923 | 5249047 |
| chr7 | 5323876 | 5324000 |
| chr7 | 5370696 | 5370829 |
| chr7 | 5488289 | 5488431 |
| chr7 | 5608854 | 5608990 |
| chr7 | 5761111 | 5761245 |
| chr7 | 6030227 | 6030375 |
| chr7 | 6120663 | 6120785 |
| chr7 | 6311428 | 6311552 |
| chr7 | 6492071 | 6492209 |
| chr7 | 6611259 | 6611381 |
| chr7 | 6709546 | 6709676 |
| chr7 | 6870541 | 6870711 |
| chr7 | 6936605 | 6936728 |
| chr7 | 6987165 | 6987289 |
| chr7 | 7169506 | 7169644 |
| chr7 | 7282927 | 7283051 |
| chr7 | 7409259 | 7409385 |
| chr7 | 7571969 | 7572091 |
| chr7 | 7838067 | 7838195 |
| chr7 | 7938300 | 7938424 |
| chr7 | 8003640 | 8003784 |
| chr7 | 8241953 | 8242089 |
| chr7 | 8391025 | 8391171 |
| chr7 | 8597028 | 8597162 |
| chr7 | 8703367 | 8703497 |
| chr7 | 8772112 | 8772234 |
| chr7 | 8909926 | 8910074 |
| chr7 | 9138802 | 9138928 |
| chr7 | 9249342 | 9249466 |
| chr7 | 9424676 | 9424820 |
| chr7 | 9520086 | 9520218 |
| chr7 | 9754588 | 9754712 |
| chr7 | 9832236 | 9832376 |
| chr7 | 10086785 | 10086911 |
| chr7 | 10182179 | 10182323 |
| chr7 | 10277208 | 10277334 |
| chr7 | 10549971 | 10550105 |
| chr7 | 10600738 | 10600878 |
| chr7 | 10798328 | 10798462 |
| chr7 | 10853850 | 10853982 |
| chr7 | 11052190 | 11052328 |
| chr7 | 11293959 | 11294097 |
| chr7 | 11383992 | 11384148 |
| chr7 | 11494514 | 11494636 |
| chr7 | 11653860 | 11653984 |
| chr7 | 11774695 | 11774841 |
| chr7 | 11915540 | 11915696 |
| chr7 | 12154342 | 12154492 |
| chr7 | 12318347 | 12318477 |
| chr7 | 12474583 | 12474713 |
| chr7 | 12615646 | 12615768 |
| chr7 | 12664066 | 12664192 |
| chr7 | 12901772 | 12901920 |
| chr7 | 13077005 | 13077135 |
| chr7 | 13141130 | 13141258 |
| chr7 | 13388265 | 13388407 |
| chr7 | 13473910 | 13474054 |
| chr7 | 13616896 | 13617036 |
| chr7 | 13784913 | 13785043 |
| chr7 | 13932549 | 13932687 |
| chr7 | 14075733 | 14075879 |
| chr7 | 14276663 | 14276799 |
| chr7 | 14391356 | 14391514 |
| chr7 | 14456493 | 14456649 |
| chr7 | 14712233 | 14712357 |
| chr7 | 14874305 | 14874435 |
| chr7 | 14911556 | 14911682 |
| chr7 | 15097938 | 15098062 |
| chr7 | 15340291 | 15340415 |
| chr7 | 15399175 | 15399315 |
| chr7 | 15643285 | 15643427 |
| chr7 | 15776493 | 15776619 |
| chr7 | 15898671 | 15898797 |
| chr7 | 16062906 | 16063028 |
| chr7 | 16132875 | 16133001 |
| chr7 | 16297295 | 16297449 |
| chr7 | 16514156 | 16514300 |
| chr7 | 16637871 | 16637995 |
| chr7 | 16704178 | 16704306 |
| chr7 | 16876716 | 16876858 |
| chr7 | 17040146 | 17040292 |
| chr7 | 17161034 | 17161164 |
| chr7 | 17436055 | 17436185 |
| chr7 | 17532112 | 17532246 |
| chr7 | 17651493 | 17651627 |
| chr7 | 17760709 | 17760867 |
| chr7 | 17920525 | 17920649 |
| chr7 | 18134568 | 18134690 |
| chr7 | 18253288 | 18253466 |
| chr7 | 18428781 | 18428905 |
| chr7 | 18648478 | 18648610 |
| chr7 | 18751908 | 18752052 |
| chr7 | 18891169 | 18891315 |
| chr7 | 18958481 | 18958603 |
| chr7 | 19209879 | 19210013 |
| chr7 | 19290852 | 19290984 |
| chr7 | 19422935 | 19423057 |
| chr7 | 19618287 | 19618435 |
| chr7 | 19781473 | 19781611 |
| chr7 | 19915619 | 19915763 |
| chr7 | 20124887 | 20125035 |
| chr7 | 20289454 | 20289578 |
| chr7 | 20347500 | 20347654 |
| chr7 | 20533952 | 20534074 |
| chr7 | 20626252 | 20626378 |
| chr7 | 20768857 | 20768981 |
| chr7 | 20977926 | 20978050 |
| chr7 | 21085476 | 21085620 |
| chr7 | 21344594 | 21344740 |
| chr7 | 21462768 | 21462904 |
| chr7 | 21526547 | 21526671 |
| chr7 | 21720073 | 21720207 |
| chr7 | 21845198 | 21845322 |
| chr7 | 22099118 | 22099266 |
| chr7 | 22250013 | 22250149 |
| chr7 | 22374844 | 22374972 |
| chr7 | 22495229 | 22495373 |
| chr7 | 22659010 | 22659134 |
| chr7 | 22757779 | 22757929 |
| chr7 | 22889193 | 22889341 |
| chr7 | 23127996 | 23128144 |
| chr7 | 23153801 | 23153935 |
| chr7 | 23419503 | 23419651 |
| chr7 | 23516068 | 23516315 |
| chr7 | 23686711 | 23686833 |
| chr7 | 23892414 | 23892548 |
| chr7 | 23921141 | 23921265 |
| chr7 | 24071333 | 24071459 |
| chr7 | 24213304 | 24213428 |
| chr7 | 24405791 | 24405925 |
| chr7 | 24617212 | 24617340 |
| chr7 | 24725956 | 24726102 |
| chr7 | 24842137 | 24842291 |
| chr7 | 25089893 | 25090015 |
| chr7 | 25216518 | 25216660 |
| chr7 | 25265360 | 25265490 |
| chr7 | 25460347 | 25460471 |
| chr7 | 25619074 | 25619208 |
| chr7 | 25752920 | 25753042 |
| chr7 | 25914761 | 25914899 |
| chr7 | 26053336 | 26053472 |
| chr7 | 26252577 | 26252701 |
| chr7 | 26334265 | 26334391 |
| chr7 | 26507997 | 26508125 |
| chr7 | 26685089 | 26685247 |
| chr7 | 26795192 | 26795322 |
| chr7 | 26968801 | 26968963 |
| chr7 | 27131499 | 27131625 |
| chr7 | 27322042 | 27322194 |
| chr7 | 27452219 | 27452379 |
| chr7 | 27522440 | 27522570 |
| chr7 | 27717640 | 27717766 |
| chr7 | 27844601 | 27844725 |
| chr7 | 28093926 | 28094066 |
| chr7 | 28203503 | 28203631 |
| chr7 | 28277764 | 28277888 |
| chr7 | 28468245 | 28468367 |
| chr7 | 28626336 | 28626464 |
| chr7 | 28708766 | 28708890 |
| chr7 | 28941467 | 28941589 |
| chr7 | 29084322 | 29084476 |
| chr7 | 29247954 | 29248076 |
| chr7 | 29314990 | 29315116 |
| chr7 | 29511615 | 29511737 |
| chr7 | 29605082 | 29605204 |
| chr7 | 29893369 | 29893501 |
| chr7 | 30025145 | 30025434 |
| chr7 | 30169194 | 30169318 |
| chr7 | 30314646 | 30314767 |
| chr7 | 30429232 | 30429356 |
| chr7 | 30595791 | 30595919 |
| chr7 | 30790104 | 30790230 |
| chr7 | 30935666 | 30935800 |
| chr7 | 31043114 | 31043236 |
| chr7 | 31149007 | 31149133 |
| chr7 | 31282523 | 31282653 |
| chr7 | 31415167 | 31415313 |
| chr7 | 31623686 | 31623816 |
| chr7 | 31799974 | 31800102 |
| chr7 | 31974334 | 31974456 |
| chr7 | 32059267 | 32059395 |
| chr7 | 32220419 | 32220549 |
| chr7 | 32452503 | 32452629 |
| chr7 | 32577662 | 32577814 |
| chr7 | 32683032 | 32683176 |
| chr7 | 32754739 | 32754863 |
| chr7 | 32803824 | 32803954 |
| chr7 | 32935150 | 32935276 |
| chr7 | 33152718 | 33152844 |
| chr7 | 33304128 | 33304252 |
| chr7 | 33389631 | 33389763 |
| chr7 | 33549286 | 33549408 |
| chr7 | 33701121 | 33701261 |
| chr7 | 33893381 | 33893503 |
| chr7 | 34005556 | 34005680 |
| chr7 | 34167958 | 34168088 |
| chr7 | 34373686 | 34373814 |
| chr7 | 34532582 | 34532720 |
| chr7 | 34605527 | 34605651 |
| chr7 | 34788088 | 34788228 |
| chr7 | 34941864 | 34941985 |
| chr7 | 35056202 | 35056348 |
| chr7 | 35185819 | 35185949 |
| chr7 | 35240338 | 35240460 |
| chr7 | 35417752 | 35417884 |
| chr7 | 35485923 | 35486063 |
| chr7 | 35687643 | 35687775 |
| chr7 | 35873456 | 35873600 |
| chr7 | 36020846 | 36021018 |
| chr7 | 36085021 | 36085149 |
| chr7 | 36241686 | 36241810 |
| chr7 | 36371461 | 36371603 |
| chr7 | 36530358 | 36530480 |
| chr7 | 36717774 | 36717896 |
| chr7 | 36945190 | 36945332 |
| chr7 | 37047717 | 37047841 |
| chr7 | 37117012 | 37117277 |
| chr7 | 37316399 | 37316539 |
| chr7 | 37516180 | 37516318 |
| chr7 | 37663407 | 37663547 |
| chr7 | 37741229 | 37741375 |
| chr7 | 37991136 | 37991268 |
| chr7 | 38142788 | 38142914 |
| chr7 | 38295183 | 38295311 |
| chr7 | 38318506 | 38318642 |
| chr7 | 38534351 | 38534481 |
| chr7 | 38668710 | 38668858 |
| chr7 | 38761442 | 38761586 |
| chr7 | 38954195 | 38954335 |
| chr7 | 39135491 | 39135625 |
| chr7 | 39302541 | 39302663 |
| chr7 | 39493435 | 39493571 |
| chr7 | 39527270 | 39527392 |
| chr7 | 39795427 | 39795680 |
| chr7 | 39886907 | 39887047 |
| chr7 | 40055024 | 40055170 |
| chr7 | 40196869 | 40197003 |
| chr7 | 40348035 | 40348169 |
| chr7 | 40466126 | 40466248 |
| chr7 | 40637580 | 40637865 |
| chr7 | 40801816 | 40801946 |
| chr7 | 40975426 | 40975550 |
| chr7 | 41082478 | 41082628 |
| chr7 | 41294616 | 41294772 |
| chr7 | 41389109 | 41389261 |
| chr7 | 41503530 | 41503672 |
| chr7 | 41649142 | 41649274 |
| chr7 | 41901614 | 41901752 |
| chr7 | 42028508 | 42028644 |
| chr7 | 42102624 | 42102746 |
| chr7 | 42217056 | 42217188 |
| chr7 | 42366254 | 42366386 |
| chr7 | 42524813 | 42524935 |
| chr7 | 42704060 | 42704186 |
| chr7 | 42916433 | 42916554 |
| chr7 | 43094614 | 43094738 |
| chr7 | 43229350 | 43229498 |
| chr7 | 43373596 | 43373736 |
| chr7 | 43550787 | 43550909 |
| chr7 | 43580130 | 43580254 |
| chr7 | 43748325 | 43748471 |
| chr7 | 43973813 | 43973955 |
| chr7 | 44048782 | 44048904 |
| chr7 | 44087215 | 44087355 |
| chr7 | 44245301 | 44245449 |
| chr7 | 44339318 | 44339442 |
| chr7 | 44584515 | 44584639 |
| chr7 | 44732015 | 44732232 |
| chr7 | 44784223 | 44784347 |
| chr7 | 45025506 | 45025628 |
| chr7 | 45085898 | 45086024 |
| chr7 | 45303005 | 45303149 |
| chr7 | 45443341 | 45443487 |
| chr7 | 45575877 | 45576001 |
| chr7 | 45670946 | 45671082 |
| chr7 | 45913829 | 45913961 |
| chr7 | 46068170 | 46068300 |
| chr7 | 46188161 | 46188303 |
| chr7 | 46261247 | 46261389 |
| chr7 | 46551516 | 46551640 |
| chr7 | 46604681 | 46604831 |
| chr7 | 46844713 | 46844835 |
| chr7 | 46959554 | 46959678 |
| chr7 | 47107899 | 47108053 |
| chr7 | 47224713 | 47224859 |
| chr7 | 47305927 | 47306051 |
| chr7 | 47569398 | 47569522 |
| chr7 | 47672426 | 47672552 |
| chr7 | 47789864 | 47790006 |
| chr7 | 47986752 | 47986892 |
| chr7 | 48152293 | 48152421 |
| chr7 | 48341330 | 48341470 |
| chr7 | 48437901 | 48438033 |
| chr7 | 48528029 | 48528151 |
| chr7 | 48795084 | 48795216 |
| chr7 | 48911101 | 48911239 |
| chr7 | 49077580 | 49077710 |
| chr7 | 49114107 | 49114229 |
| chr7 | 49373727 | 49373849 |
| chr7 | 49429902 | 49430026 |
| chr7 | 49639326 | 49639456 |
| chr7 | 49821248 | 49821372 |
| chr7 | 49983290 | 49983432 |
| chr7 | 50141439 | 50141585 |
| chr7 | 50281477 | 50281613 |
| chr7 | 50350177 | 50350303 |
| chr7 | 50462070 | 50462224 |
| chr7 | 50664864 | 50665008 |
| chr7 | 50806991 | 50807119 |
| chr7 | 50933286 | 50933414 |
| chr7 | 51182408 | 51182534 |
| chr7 | 51293991 | 51294117 |
| chr7 | 51375405 | 51375543 |
| chr7 | 51605057 | 51605179 |
| chr7 | 51697245 | 51697403 |
| chr7 | 51910316 | 51910454 |
| chr7 | 51954708 | 51954830 |
| chr7 | 52151266 | 52151392 |
| chr7 | 52328152 | 52328294 |
| chr7 | 52537005 | 52537141 |
| chr7 | 52684358 | 52684494 |
| chr7 | 52842355 | 52842493 |
| chr7 | 52997146 | 52997270 |
| chr7 | 53062068 | 53062190 |
| chr7 | 53297019 | 53297149 |
| chr7 | 53377963 | 53378093 |
| chr7 | 53603248 | 53603372 |
| chr7 | 53706459 | 53706601 |
| chr7 | 53779015 | 53779139 |
| chr7 | 53945555 | 53945679 |
| chr7 | 54137460 | 54137582 |
| chr7 | 54271464 | 54271588 |
| chr7 | 54430679 | 54430805 |
| chr7 | 54619084 | 54619206 |
| chr7 | 54767594 | 54767718 |
| chr7 | 54926106 | 54926254 |
| chr7 | 54961665 | 54961789 |
| chr7 | 55173609 | 55173759 |
| chr7 | 55292793 | 55292931 |
| chr7 | 55482757 | 55482883 |
| chr7 | 55607125 | 55607247 |
| chr7 | 55724403 | 55724525 |
| chr7 | 55890112 | 55890234 |
| chr7 | 56038766 | 56038910 |
| chr7 | 56291836 | 56291972 |
| chr7 | 56382902 | 56383033 |
| chr7 | 56584918 | 56585042 |
| chr7 | 56630535 | 56630657 |
| chr7 | 56794634 | 56794756 |
| chr7 | 56854207 | 56854357 |
| chr7 | 56927392 | 56927534 |
| chr7 | 56928778 | 56928919 |
| chr7 | 57015533 | 57015657 |
| chr7 | 57164082 | 57164210 |
| chr7 | 57323668 | 57323792 |
| chr7 | 57488722 | 57488862 |
| chr7 | 57558103 | 57558253 |
| chr7 | 57643195 | 57643339 |
| chr7 | 61062477 | 61062615 |
| chr7 | 61804055 | 61804209 |
| chr7 | 61914080 | 61914206 |
| chr7 | 62030285 | 62030409 |
| chr7 | 62282666 | 62282796 |
| chr7 | 62460883 | 62461148 |
| chr7 | 62678336 | 62678457 |
| chr7 | 62728299 | 62728427 |
| chr7 | 62827734 | 62827856 |
| chr7 | 63089382 | 63089506 |
| chr7 | 63176082 | 63176224 |
| chr7 | 63177470 | 63177612 |
| chr7 | 63251692 | 63251842 |
| chr7 | 63398331 | 63398452 |
| chr7 | 63514729 | 63514877 |
| chr7 | 63745140 | 63745314 |
| chr7 | 63918379 | 63918503 |
| chr7 | 64019516 | 64019646 |
| chr7 | 64116863 | 64116987 |
| chr7 | 64366589 | 64366711 |
| chr7 | 64406403 | 64406525 |
| chr7 | 64692274 | 64692414 |
| chr7 | 64807205 | 64807349 |
| chr7 | 64866056 | 64866198 |
| chr7 | 65084353 | 65084475 |
| chr7 | 65269444 | 65269729 |
| chr7 | 65375433 | 65375563 |
| chr7 | 65489520 | 65489672 |
| chr7 | 65639770 | 65639914 |
| chr7 | 65825923 | 65826069 |
| chr7 | 65935695 | 65935815 |
| chr7 | 66029188 | 66029309 |
| chr7 | 66112679 | 66112823 |
| chr7 | 66213405 | 66213529 |
| chr7 | 66487576 | 66487726 |
| chr7 | 66555580 | 66555704 |
| chr7 | 66579248 | 66579370 |
| chr7 | 66671891 | 66672139 |
| chr7 | 66699444 | 66699588 |
| chr7 | 66753020 | 66753144 |
| chr7 | 66883322 | 66883448 |
| chr7 | 67006469 | 67006627 |
| chr7 | 67137474 | 67137596 |
| chr7 | 67339954 | 67340102 |
| chr7 | 67490255 | 67490409 |
| chr7 | 67558316 | 67558468 |
| chr7 | 67716253 | 67716377 |
| chr7 | 67973810 | 67973944 |
| chr7 | 68132524 | 68132654 |
| chr7 | 68270951 | 68271075 |
| chr7 | 68373581 | 68373705 |
| chr7 | 68583458 | 68583586 |
| chr7 | 68682886 | 68683014 |
| chr7 | 68872324 | 68872452 |
| chr7 | 68905590 | 68905712 |
| chr7 | 69168355 | 69168481 |
| chr7 | 69220519 | 69220647 |
| chr7 | 69435041 | 69435167 |
| chr7 | 69564290 | 69564450 |
| chr7 | 69766505 | 69766637 |
| chr7 | 69813951 | 69814081 |
| chr7 | 69982066 | 69982236 |
| chr7 | 70218641 | 70218765 |
| chr7 | 70260011 | 70260137 |
| chr7 | 70461957 | 70462087 |
| chr7 | 70619897 | 70620021 |
| chr7 | 70785851 | 70785975 |
| chr7 | 70935384 | 70935528 |
| chr7 | 71020253 | 71020377 |
| chr7 | 71285937 | 71286059 |
| chr7 | 71393279 | 71393417 |
| chr7 | 71588594 | 71588732 |
| chr7 | 71649212 | 71649350 |
| chr7 | 71813584 | 71813728 |
| chr7 | 71905070 | 71905194 |
| chr7 | 72005849 | 72005971 |
| chr7 | 72044405 | 72044549 |
| chr7 | 72162887 | 72163009 |
| chr7 | 72186603 | 72186724 |
| chr7 | 72216279 | 72216400 |
| chr7 | 72332932 | 72333056 |
| chr7 | 72365733 | 72365950 |
| chr7 | 72408876 | 72409028 |
| chr7 | 72499243 | 72499385 |
| chr7 | 72527305 | 72527447 |
| chr7 | 72749992 | 72750114 |
| chr7 | 72845621 | 72845742 |
| chr7 | 72970532 | 72970653 |
| chr7 | 73213624 | 73213764 |
| chr7 | 73307223 | 73307349 |
| chr7 | 73437891 | 73438013 |
| chr7 | 73564020 | 73564142 |
| chr7 | 73780722 | 73780868 |
| chr7 | 73864073 | 73864194 |
| chr7 | 74136410 | 74136753 |
| chr7 | 74330093 | 74330235 |
| chr7 | 74421255 | 74421383 |
| chr7 | 74485773 | 74485991 |
| chr7 | 74825029 | 74825157 |
| chr7 | 74909438 | 74909580 |
| chr7 | 74937456 | 74937598 |
| chr7 | 74965483 | 74965625 |
| chr7 | 75062023 | 75062144 |
| chr7 | 75084390 | 75084518 |
| chr7 | 75132673 | 75132797 |
| chr7 | 75160907 | 75161028 |
| chr7 | 75255680 | 75255826 |
| chr7 | 75372903 | 75373029 |
| chr7 | 75546073 | 75546215 |
| chr7 | 75775923 | 75776061 |
| chr7 | 75880140 | 75880278 |
| chr7 | 76037082 | 76037210 |
| chr7 | 76137382 | 76137502 |
| chr7 | 76161471 | 76161595 |
| chr7 | 76214203 | 76214349 |
| chr7 | 76269045 | 76269173 |
| chr7 | 76391324 | 76391472 |
| chr7 | 76539802 | 76539940 |
| chr7 | 76652475 | 76652597 |
| chr7 | 76660617 | 76660759 |
| chr7 | 76680556 | 76680680 |
| chr7 | 76707484 | 76707610 |
| chr7 | 76752126 | 76752256 |
| chr7 | 76844179 | 76844303 |
| chr7 | 76941754 | 76941888 |
| chr7 | 77106592 | 77106716 |
| chr7 | 77223205 | 77223345 |
| chr7 | 77419252 | 77419376 |
| chr7 | 77541587 | 77541721 |
| chr7 | 77628089 | 77628215 |
| chr7 | 77778586 | 77778712 |
| chr7 | 78022536 | 78022660 |
| chr7 | 78068415 | 78068561 |
| chr7 | 78324788 | 78324936 |
| chr7 | 78438592 | 78438716 |
| chr7 | 78564881 | 78565023 |
| chr7 | 78783039 | 78783169 |
| chr7 | 78869138 | 78869280 |
| chr7 | 79004184 | 79004308 |
| chr7 | 79246555 | 79246683 |
| chr7 | 79316122 | 79316248 |
| chr7 | 79541501 | 79541655 |
| chr7 | 79618956 | 79619084 |
| chr7 | 79792580 | 79792712 |
| chr7 | 79908225 | 79908349 |
| chr7 | 80068872 | 80068996 |
| chr7 | 80258770 | 80258898 |
| chr7 | 80424029 | 80424153 |
| chr7 | 80473980 | 80474132 |
| chr7 | 80697542 | 80697684 |
| chr7 | 80837329 | 80837457 |
| chr7 | 80978490 | 80978612 |
| chr7 | 81161259 | 81161380 |
| chr7 | 81279902 | 81280024 |
| chr7 | 81404161 | 81404285 |
| chr7 | 81535266 | 81535414 |
| chr7 | 81671201 | 81671341 |
| chr7 | 81917983 | 81918127 |
| chr7 | 82097051 | 82097215 |
| chr7 | 82204555 | 82204701 |
| chr7 | 82376916 | 82377038 |
| chr7 | 82534647 | 82534785 |
| chr7 | 82689833 | 82689987 |
| chr7 | 82846726 | 82846858 |
| chr7 | 82990897 | 82991035 |
| chr7 | 83068388 | 83068512 |
| chr7 | 83236359 | 83236487 |
| chr7 | 83433887 | 83434009 |
| chr7 | 83586634 | 83586758 |
| chr7 | 83734283 | 83734409 |
| chr7 | 83831248 | 83831372 |
| chr7 | 83972126 | 83972250 |
| chr7 | 84163064 | 84163186 |
| chr7 | 84205130 | 84205266 |
| chr7 | 84442740 | 84442866 |
| chr7 | 84631324 | 84631474 |
| chr7 | 84657971 | 84658099 |
| chr7 | 84866584 | 84866730 |
| chr7 | 85099834 | 85099980 |
| chr7 | 85206576 | 85206704 |
| chr7 | 85364888 | 85365018 |
| chr7 | 85513460 | 85513582 |
| chr7 | 85564769 | 85564893 |
| chr7 | 85820872 | 85821002 |
| chr7 | 85897270 | 85897394 |
| chr7 | 86026444 | 86026592 |
| chr7 | 86189179 | 86189305 |
| chr7 | 86395850 | 86395988 |
| chr7 | 86558592 | 86558722 |
| chr7 | 86609877 | 86610025 |
| chr7 | 86885053 | 86885207 |
| chr7 | 86979441 | 86979571 |
| chr7 | 87110586 | 87110728 |
| chr7 | 87282650 | 87282771 |
| chr7 | 87412794 | 87412915 |
| chr7 | 87530957 | 87531142 |
| chr7 | 87752454 | 87752576 |
| chr7 | 87828114 | 87828238 |
| chr7 | 88021478 | 88021600 |
| chr7 | 88163218 | 88163346 |
| chr7 | 88270630 | 88270760 |
| chr7 | 88522535 | 88522675 |
| chr7 | 88701185 | 88701343 |
| chr7 | 88708445 | 88708573 |
| chr7 | 88904632 | 88904760 |
| chr7 | 89073213 | 89073357 |
| chr7 | 89295241 | 89295365 |
| chr7 | 89410062 | 89410186 |
| chr7 | 89552899 | 89553025 |
| chr7 | 89645055 | 89645179 |
| chr7 | 89816653 | 89816777 |
| chr7 | 90023017 | 90023167 |
| chr7 | 90122548 | 90122682 |
| chr7 | 90236145 | 90236289 |
| chr7 | 90354550 | 90354680 |
| chr7 | 90522479 | 90522611 |
| chr7 | 90716418 | 90716570 |
| chr7 | 90835758 | 90835884 |
| chr7 | 91047064 | 91047204 |
| chr7 | 91116639 | 91116763 |
| chr7 | 91403065 | 91403211 |
| chr7 | 91417730 | 91417856 |
| chr7 | 91644499 | 91644623 |
| chr7 | 91825116 | 91825238 |
| chr7 | 91896240 | 91896376 |
| chr7 | 92029244 | 92029372 |
| chr7 | 92187224 | 92187346 |
| chr7 | 92303518 | 92303642 |
| chr7 | 92533232 | 92533372 |
| chr7 | 92643323 | 92643447 |
| chr7 | 92897424 | 92897568 |
| chr7 | 92915636 | 92915760 |
| chr7 | 93098444 | 93098568 |
| chr7 | 93340526 | 93340652 |
| chr7 | 93485080 | 93485204 |
| chr7 | 93632926 | 93633062 |
| chr7 | 93693843 | 93693987 |
| chr7 | 93938741 | 93938867 |
| chr7 | 94031405 | 94031527 |
| chr7 | 94224196 | 94224324 |
| chr7 | 94276897 | 94277229 |
| chr7 | 94552421 | 94552547 |
| chr7 | 94568117 | 94568247 |
| chr7 | 94735692 | 94735830 |
| chr7 | 94867729 | 94867875 |
| chr7 | 95026683 | 95026841 |
| chr7 | 95240922 | 95241044 |
| chr7 | 95399240 | 95399408 |
| chr7 | 95589947 | 95590075 |
| chr7 | 95648049 | 95648173 |
| chr7 | 95766791 | 95766915 |
| chr7 | 95967119 | 95967279 |
| chr7 | 96054488 | 96054628 |
| chr7 | 96313520 | 96313662 |
| chr7 | 96428186 | 96428324 |
| chr7 | 96517605 | 96517753 |
| chr7 | 96735703 | 96735835 |
| chr7 | 96902584 | 96902722 |
| chr7 | 97079829 | 97079965 |
| chr7 | 97126010 | 97126134 |
| chr7 | 97260358 | 97260482 |
| chr7 | 97532227 | 97532373 |
| chr7 | 97676459 | 97676609 |
| chr7 | 97755008 | 97755154 |
| chr7 | 97904421 | 97904555 |
| chr7 | 98111169 | 98111319 |
| chr7 | 98280125 | 98280249 |
| chr7 | 98413610 | 98413742 |
| chr7 | 98473160 | 98473300 |
| chr7 | 98713870 | 98714010 |
| chr7 | 98836826 | 98836982 |
| chr7 | 99039613 | 99039857 |
| chr7 | 99150382 | 99150562 |
| chr7 | 99332019 | 99332163 |
| chr7 | 99392595 | 99392872 |
| chr7 | 99583216 | 99583374 |
| chr7 | 99736007 | 99736131 |
| chr7 | 99904614 | 99904755 |
| chr7 | 99919020 | 99919142 |
| chr7 | 99944614 | 99944786 |
| chr7 | 99973405 | 99973665 |
| chr7 | 100108398 | 100108645 |
| chr7 | 100289957 | 100290089 |
| chr7 | 100476020 | 100476174 |
| chr7 | 100626901 | 100627041 |
| chr7 | 100727356 | 100727494 |
| chr7 | 100954739 | 100954887 |
| chr7 | 101041704 | 101041828 |
| chr7 | 101224105 | 101224253 |
| chr7 | 101412835 | 101412967 |
| chr7 | 101591519 | 101591649 |
| chr7 | 101658161 | 101658295 |
| chr7 | 101842479 | 101842629 |
| chr7 | 101931639 | 101931775 |
| chr7 | 101987028 | 101987151 |
| chr7 | 102076623 | 102076763 |
| chr7 | 102203012 | 102203136 |
| chr7 | 102302102 | 102302226 |
| chr7 | 102388843 | 102388973 |
| chr7 | 102433511 | 102433637 |
| chr7 | 102551861 | 102552003 |
| chr7 | 102693885 | 102694012 |
| chr7 | 102879421 | 102879551 |
| chr7 | 103023436 | 103023625 |
| chr7 | 103229237 | 103229363 |
| chr7 | 103397630 | 103397754 |
| chr7 | 103407640 | 103407764 |
| chr7 | 103555180 | 103555304 |
| chr7 | 103752653 | 103752779 |
| chr7 | 103904357 | 103904481 |
| chr7 | 104008455 | 104008581 |
| chr7 | 104178282 | 104178406 |
| chr7 | 104347754 | 104347882 |
| chr7 | 104535482 | 104535630 |
| chr7 | 104710077 | 104710209 |
| chr7 | 104890880 | 104891008 |
| chr7 | 104948040 | 104948164 |
| chr7 | 105123060 | 105123184 |
| chr7 | 105221045 | 105221181 |
| chr7 | 105354644 | 105354768 |
| chr7 | 105561051 | 105561187 |
| chr7 | 105741239 | 105741369 |
| chr7 | 105901617 | 105901741 |
| chr7 | 106009282 | 106009410 |
| chr7 | 106160820 | 106160950 |
| chr7 | 106287691 | 106287827 |
| chr7 | 106545164 | 106545314 |
| chr7 | 106591734 | 106591858 |
| chr7 | 106713741 | 106713863 |
| chr7 | 106860825 | 106860946 |
| chr7 | 107119124 | 107119258 |
| chr7 | 107224595 | 107224737 |
| chr7 | 107404402 | 107404526 |
| chr7 | 107500942 | 107501074 |
| chr7 | 107686135 | 107686256 |
| chr7 | 107785075 | 107785205 |
| chr7 | 108021791 | 108021923 |
| chr7 | 108076915 | 108077039 |
| chr7 | 108248808 | 108248932 |
| chr7 | 108455712 | 108455835 |
| chr7 | 108574605 | 108574753 |
| chr7 | 108713817 | 108713977 |
| chr7 | 108932121 | 108932259 |
| chr7 | 109099632 | 109099760 |
| chr7 | 109186678 | 109186806 |
| chr7 | 109254880 | 109255006 |
| chr7 | 109492248 | 109492380 |
| chr7 | 109661944 | 109662078 |
| chr7 | 109828004 | 109828148 |
| chr7 | 109969096 | 109969220 |
| chr7 | 110074186 | 110074310 |
| chr7 | 110196526 | 110196704 |
| chr7 | 110340510 | 110340686 |
| chr7 | 110472571 | 110472727 |
| chr7 | 110722168 | 110722320 |
| chr7 | 110858220 | 110858346 |
| chr7 | 111044115 | 111044247 |
| chr7 | 111169760 | 111169890 |
| chr7 | 111326190 | 111326354 |
| chr7 | 111472988 | 111473112 |
| chr7 | 111517428 | 111517558 |
| chr7 | 111740633 | 111740769 |
| chr7 | 111843256 | 111843394 |
| chr7 | 112098459 | 112098593 |
| chr7 | 112207232 | 112207354 |
| chr7 | 112321529 | 112321651 |
| chr7 | 112464789 | 112464927 |
| chr7 | 112655422 | 112655546 |
| chr7 | 112839168 | 112839292 |
| chr7 | 112956062 | 112956198 |
| chr7 | 113139729 | 113139851 |
| chr7 | 113270089 | 113270229 |
| chr7 | 113349086 | 113349210 |
| chr7 | 113515311 | 113515437 |
| chr7 | 113741723 | 113741847 |
| chr7 | 113887644 | 113887770 |
| chr7 | 113938691 | 113938819 |
| chr7 | 114167990 | 114168111 |
| chr7 | 114258138 | 114258261 |
| chr7 | 114421157 | 114421295 |
| chr7 | 114587339 | 114587473 |
| chr7 | 114739562 | 114739688 |
| chr7 | 114915468 | 114915592 |
| chr7 | 114953511 | 114953655 |
| chr7 | 115199165 | 115199303 |
| chr7 | 115311763 | 115311891 |
| chr7 | 115527004 | 115527136 |
| chr7 | 115625534 | 115625668 |
| chr7 | 115805187 | 115805327 |
| chr7 | 115860741 | 115860867 |
| chr7 | 116003922 | 116004078 |
| chr7 | 116266642 | 116266764 |
| chr7 | 116399041 | 116399165 |
| chr7 | 116546927 | 116547049 |
| chr7 | 116609480 | 116609734 |
| chr7 | 116756337 | 116756605 |
| chr7 | 116935525 | 116935649 |
| chr7 | 117165111 | 117165233 |
| chr7 | 117335103 | 117335243 |
| chr7 | 117477064 | 117477196 |
| chr7 | 117547624 | 117547768 |
| chr7 | 117682011 | 117682189 |
| chr7 | 117808348 | 117808472 |
| chr7 | 117976237 | 117976363 |
| chr7 | 118106150 | 118106274 |
| chr7 | 118390854 | 118390996 |
| chr7 | 118493398 | 118493524 |
| chr7 | 118702841 | 118702970 |
| chr7 | 118781346 | 118781476 |
| chr7 | 118984799 | 118984924 |
| chr7 | 119112538 | 119112664 |
| chr7 | 119215166 | 119215296 |
| chr7 | 119341438 | 119341698 |
| chr7 | 119473082 | 119473295 |
| chr7 | 119740632 | 119740756 |
| chr7 | 119804387 | 119804509 |
| chr7 | 119930436 | 119930560 |
| chr7 | 120136894 | 120137032 |
| chr7 | 120317888 | 120318012 |
| chr7 | 120461866 | 120462000 |
| chr7 | 120521330 | 120521468 |
| chr7 | 120670962 | 120671120 |
| chr7 | 120817063 | 120817201 |
| chr7 | 120974729 | 120974853 |
| chrz | 121246206 | 121246344 |
| chr7 | 121323586 | 121323712 |
| chr7 | 121410119 | 121410251 |
| chr7 | 121564962 | 121565088 |
| chr7 | 121728390 | 121728645 |
| chr7 | 121992213 | 121992347 |
| chr7 | 122077127 | 122077253 |
| chr7 | 122186644 | 122186774 |
| chr7 | 122439265 | 122439387 |
| chr7 | 122505216 | 122505337 |
| chr7 | 122651942 | 122652082 |
| chr7 | 122819817 | 122819959 |
| chr7 | 122950022 | 122950156 |
| chr7 | 123195702 | 123195824 |
| chr7 | 123285388 | 123285512 |
| chr7 | 123393404 | 123393532 |
| chr7 | 123638942 | 123639064 |
| chr7 | 123703942 | 123704074 |
| chr7 | 123860187 | 123860325 |
| chr7 | 124095688 | 124095818 |
| chr7 | 124138119 | 124138251 |
| chr7 | 124307850 | 124307984 |
| chr7 | 124410533 | 124410655 |
| chr7 | 124571014 | 124571168 |
| chr7 | 124739739 | 124739895 |
| chr7 | 124930551 | 124930691 |
| chr7 | 125115332 | 125115470 |
| chr7 | 125270573 | 125270701 |
| chr7 | 125366328 | 125366462 |
| chr7 | 125577766 | 125577892 |
| chr7 | 125684699 | 125684823 |
| chr7 | 125883800 | 125883948 |
| chr7 | 125986795 | 125986935 |
| chr7 | 126186255 | 126186379 |
| chr7 | 126225310 | 126225442 |
| chr7 | 126356293 | 126356425 |
| chr7 | 126542190 | 126542308 |
| chr7 | 126566468 | 126566595 |
| chr7 | 126676312 | 126676438 |
| chr7 | 126838357 | 126838507 |
| chr7 | 127048653 | 127048795 |
| chr7 | 127158883 | 127159023 |
| chr7 | 127263828 | 127263972 |
| chr7 | 127472077 | 127472199 |
| chr7 | 127667464 | 127667604 |
| chr7 | 127754948 | 127755072 |
| chr7 | 127982279 | 127982419 |
| chr7 | 128039760 | 128039904 |
| chr7 | 128174297 | 128174419 |
| chr7 | 128403753 | 128403877 |
| chr7 | 128575466 | 128575608 |
| chr7 | 128683489 | 128683623 |
| chr7 | 128877245 | 128877387 |
| chr7 | 128904481 | 128904609 |
| chr7 | 129144128 | 129144280 |
| chr7 | 129253791 | 129253935 |
| chr7 | 129354823 | 129354947 |
| chr7 | 129593265 | 129593387 |
| chr7 | 129726655 | 129726809 |
| chr7 | 129854717 | 129854841 |
| chr7 | 130034619 | 130034747 |
| chr7 | 130144354 | 130144488 |
| chr7 | 130371279 | 130371407 |
| chr7 | 130412608 | 130412748 |
| chr7 | 130688521 | 130688647 |
| chr7 | 130815323 | 130815459 |
| chr7 | 130923779 | 130923907 |
| chr7 | 131133624 | 131133778 |
| chr7 | 131298031 | 131298159 |
| chr7 | 131395320 | 131395450 |
| chr7 | 131491669 | 131491819 |
| chr7 | 131662718 | 131662852 |
| chr7 | 131770085 | 131770221 |
| chr7 | 131914128 | 131914252 |
| chr7 | 132186465 | 132186593 |
| chr7 | 132304287 | 132304443 |
| chr7 | 132396214 | 132396336 |
| chr7 | 132596182 | 132596310 |
| chr7 | 132689213 | 132689355 |
| chr7 | 132831113 | 132831241 |
| chr7 | 132998043 | 132998201 |
| chr7 | 133200765 | 133200891 |
| chr7 | 133260617 | 133260765 |
| chr7 | 133440710 | 133440834 |
| chr7 | 133680541 | 133680677 |
| chr7 | 133811255 | 133811379 |
| chr7 | 133964290 | 133964420 |
| chr7 | 134065294 | 134065416 |
| chr7 | 134178920 | 134179044 |
| chr7 | 134342232 | 134342354 |
| chr7 | 134480551 | 134480675 |
| chr7 | 134746505 | 134746631 |
| chr7 | 134793955 | 134794099 |
| chr7 | 134991597 | 134991721 |
| chr7 | 135119361 | 135119487 |
| chr7 | 135242970 | 135243116 |
| chr7 | 135391794 | 135391918 |
| chr7 | 135596825 | 135596951 |
| chr7 | 135776716 | 135776860 |
| chr7 | 135942325 | 135942451 |
| chr7 | 136064281 | 136064409 |
| chr7 | 136243378 | 136243508 |
| chr7 | 136388952 | 136389074 |
| chr7 | 136484953 | 136485091 |
| chr7 | 136633131 | 136633265 |
| chr7 | 136812402 | 136812530 |
| chr7 | 136956060 | 136956214 |
| chr7 | 137141192 | 137141316 |
| chr7 | 137201702 | 137201848 |
| chr7 | 137426506 | 137426650 |
| chr7 | 137457066 | 137457202 |
| chr7 | 137615156 | 137615292 |
| chr7 | 137890141 | 137890265 |
| chr7 | 138033113 | 138033247 |
| chr7 | 138157354 | 138157478 |
| chr7 | 138278857 | 138279001 |
| chr7 | 138386499 | 138386639 |
| chr7 | 138579981 | 138580103 |
| chr7 | 138662933 | 138663057 |
| chr7 | 138881225 | 138881429 |
| chr7 | 138990868 | 138991138 |
| chr7 | 139112622 | 139112758 |
| chr7 | 139373922 | 139374064 |
| chr7 | 139462455 | 139462581 |
| chr7 | 139617915 | 139618053 |
| chr7 | 139831085 | 139831207 |
| chr7 | 139970114 | 139970246 |
| chr7 | 140108040 | 140108176 |
| chr7 | 140277788 | 140277928 |
| chr7 | 140400618 | 140400740 |
| chr7 | 140601350 | 140601490 |
| chr7 | 140698485 | 140698621 |
| chr7 | 140758341 | 140758467 |
| chr7 | 140975170 | 140975294 |
| chr7 | 141054202 | 141054356 |
| chr7 | 141305747 | 141305873 |
| chr7 | 141420212 | 141420340 |
| chr7 | 141565848 | 141565992 |
| chr7 | 141694186 | 141694336 |
| chr7 | 141900038 | 141900166 |
| chr7 | 142043843 | 142043969 |
| chr7 | 142145710 | 142145834 |
| chr7 | 142273046 | 142273261 |
| chr7 | 142420265 | 142420393 |
| chr7 | 142558060 | 142558351 |
| chr7 | 142800391 | 142800591 |
| chr7 | 142944080 | 142944333 |
| chr7 | 143063982 | 143064114 |
| chr7 | 143217572 | 143217694 |
| chr7 | 143434293 | 143434450 |
| chr7 | 143547442 | 143547569 |
| chr7 | 143624889 | 143625023 |
| chr7 | 143780314 | 143780448 |
| chr7 | 143950847 | 143950977 |
| chr7 | 143994184 | 143994314 |
| chr7 | 144183658 | 144183808 |
| chr7 | 144290526 | 144290650 |
| chr7 | 144400578 | 144400710 |
| chr7 | 144549022 | 144549156 |
| chr7 | 144682061 | 144682195 |
| chr7 | 144851971 | 144852095 |
| chr7 | 145008578 | 145008702 |
| chr7 | 145130844 | 145130966 |
| chr7 | 145366322 | 145366444 |
| chr7 | 145480053 | 145480177 |
| chr7 | 145589409 | 145589533 |
| chr7 | 145809800 | 145810118 |
| chr7 | 145953093 | 145953229 |
| chr7 | 146104400 | 146104526 |
| chr7 | 146198471 | 146198595 |
| chr7 | 146439335 | 146439465 |
| chr7 | 146503203 | 146503335 |
| chr7 | 146610305 | 146610437 |
| chr7 | 146771446 | 146771572 |
| chr7 | 147014502 | 147014644 |
| chr7 | 147197530 | 147197660 |
| chr7 | 147312937 | 147313067 |
| chr7 | 147440592 | 147440716 |
| chr7 | 147559396 | 147559526 |
| chr7 | 147731327 | 147731457 |
| chr7 | 147806254 | 147806386 |
| chr7 | 148047756 | 148047878 |
| chr7 | 148246324 | 148246450 |
| chr7 | 148358023 | 148358159 |
| chr7 | 148513423 | 148513547 |
| chr7 | 148637746 | 148637872 |
| chr7 | 148738900 | 148739026 |
| chr7 | 148960761 | 148960885 |
| chr7 | 149064127 | 149064249 |
| chr7 | 149241215 | 149241341 |
| chr7 | 149424733 | 149424855 |
| chr7 | 149585470 | 149585608 |
| chr7 | 149714312 | 149714450 |
| chr7 | 149824285 | 149824435 |
| chr7 | 149884438 | 149884586 |
| chr7 | 150014891 | 150015070 |
| chr7 | 150123871 | 150124019 |
| chr7 | 150307678 | 150307806 |
| chr7 | 150392269 | 150392391 |
| chr7 | 150530432 | 150530560 |
| chr7 | 150790057 | 150790193 |
| chr7 | 150855192 | 150855314 |
| chr7 | 151019783 | 151019909 |
| chr7 | 151123451 | 151123581 |
| chr7 | 151319330 | 151319470 |
| chr7 | 151530237 | 151530363 |
| chr7 | 151668349 | 151668485 |
| chr7 | 151715885 | 151716035 |
| chr7 | 151893604 | 151893757 |
| chr7 | 152044440 | 152044562 |
| chr7 | 152079558 | 152079732 |
| chr7 | 152162119 | 152162277 |
| chr7 | 152377759 | 152377905 |
| chr7 | 152458058 | 152458178 |
| chr7 | 152694100 | 152694230 |
| chr7 | 152868536 | 152868688 |
| chr7 | 153046065 | 153046191 |
| chr7 | 153161248 | 153161392 |
| chr7 | 153209082 | 153209210 |
| chr7 | 153401031 | 153401151 |
| chr7 | 153476919 | 153477039 |
| chr7 | 153578362 | 153578504 |
| chr7 | 153670560 | 153670710 |
| chr7 | 153777040 | 153777178 |
| chr7 | 153868256 | 153868386 |
| chr7 | 153953829 | 153953965 |
| chr7 | 154123627 | 154123751 |
| chr7 | 154371986 | 154372226 |
| chr7 | 154412828 | 154412968 |
| chr7 | 154657581 | 154657705 |
| chr7 | 154823325 | 154823449 |
| chr7 | 154991556 | 154991704 |
| chr7 | 155072161 | 155072285 |
| chr7 | 155295201 | 155295361 |
| chr7 | 155344868 | 155345028 |
| chr7 | 155520855 | 155520979 |
| chr7 | 155733905 | 155734031 |
| chr7 | 155860393 | 155860521 |
| chr7 | 155926768 | 155926890 |
| chr7 | 156194097 | 156194233 |
| chr7 | 156208529 | 156208671 |
| chr7 | 156365203 | 156365339 |
| chr7 | 156551018 | 156551144 |
| chr7 | 156690377 | 156690624 |
| chr7 | 156813266 | 156813387 |
| chr7 | 156972260 | 156972384 |
| chr7 | 157134308 | 157134448 |
| chr7 | 157308074 | 157308198 |
| chr7 | 157429814 | 157429936 |
| chr7 | 157650395 | 157650521 |
| chr7 | 157706657 | 157706793 |
| chr7 | 157861700 | 157861842 |
| chr7 | 158090707 | 158090829 |
| chr7 | 158280583 | 158280715 |
| chr7 | 158342378 | 158342510 |
| chr7 | 158458494 | 158458636 |
| chr7 | 158746557 | 158746689 |
| chr7 | 158891470 | 158891594 |
| chr7 | 159046338 | 159046514 |
| chr7 | 159099673 | 159099805 |
| chr8 | 153648 | 153778 |
| chr8 | 238536 | 238660 |
| chr8 | 471753 | 471883 |
| chr8 | 479874 | 480048 |
| chr8 | 733328 | 733472 |
| chr8 | 832866 | 833018 |
| chr8 | 1033583 | 1033713 |
| chr8 | 1220812 | 1220936 |
| chr8 | 1320908 | 1321034 |
| chr8 | 1450602 | 1450728 |
| chr8 | 1582250 | 1582372 |
| chr8 | 1721036 | 1721160 |
| chr8 | 1904431 | 1904557 |
| chr8 | 2005095 | 2005217 |
| chr8 | 2235984 | 2236158 |
| chr8 | 2295715 | 2295837 |
| chr8 | 2551487 | 2551619 |
| chr8 | 2654813 | 2654945 |
| chr8 | 2844509 | 2844633 |
| chr8 | 2905654 | 2905784 |
| chr8 | 3054675 | 3054797 |
| chr8 | 3306378 | 3306512 |
| chr8 | 3403132 | 3403272 |
| chr8 | 3570085 | 3570229 |
| chr8 | 3729430 | 3729552 |
| chr8 | 3896280 | 3896420 |
| chr8 | 4076153 | 4076295 |
| chr8 | 4131404 | 4131538 |
| chr8 | 4249633 | 4249759 |
| chr8 | 4474971 | 4475103 |
| chr8 | 4637587 | 4637727 |
| chr8 | 4764781 | 4764947 |
| chr8 | 4886226 | 4886358 |
| chr8 | 5086061 | 5086185 |
| chr8 | 5190522 | 5190672 |
| chr8 | 5362985 | 5363123 |
| chr8 | 5494338 | 5494464 |
| chr8 | 5617024 | 5617152 |
| chr8 | 5846966 | 5847096 |
| chr8 | 5899716 | 5899846 |
| chr8 | 6084256 | 6084388 |
| chr8 | 6177792 | 6177916 |
| chr8 | 6444313 | 6444459 |
| chr8 | 6550572 | 6550702 |
| chr8 | 6758333 | 6758477 |
| chr8 | 6911385 | 6911507 |
| chr8 | 6976333 | 6976485 |
| chr8 | 7052955 | 7053079 |
| chr8 | 7055866 | 7056013 |
| chr8 | 7220217 | 7220339 |
| chr8 | 7230201 | 7230329 |
| chr8 | 7277039 | 7277177 |
| chr8 | 7308157 | 7308285 |
| chr8 | 7396307 | 7396457 |
| chr8 | 7639627 | 7639777 |
| chr8 | 7718304 | 7718432 |
| chr8 | 7749409 | 7749547 |
| chr8 | 7796628 | 7796756 |
| chr8 | 7946586 | 7946706 |
| chr8 | 7949474 | 7949598 |
| chr8 | 8041761 | 8041958 |
| chr8 | 8068027 | 8068153 |
| chr8 | 8099535 | 8099673 |
| chr8 | 8257979 | 8258103 |
| chr8 | 8289817 | 8289951 |
| chr8 | 8544798 | 8544920 |
| chr8 | 8648312 | 8648433 |
| chr8 | 8771068 | 8771204 |
| chr8 | 9007191 | 9007321 |
| chr8 | 9079213 | 9079341 |
| chr8 | 9281090 | 9281216 |
| chr8 | 9406528 | 9406664 |
| chr8 | 9593413 | 9593557 |
| chr8 | 9653081 | 9653225 |
| chr8 | 9813465 | 9813599 |
| chr8 | 10000233 | 10000359 |
| chr8 | 10153556 | 10153682 |
| chr8 | 10234614 | 10234744 |
| chr8 | 10391879 | 10392001 |
| chr8 | 10641323 | 10641471 |
| chr8 | 10752388 | 10752510 |
| chr8 | 10845097 | 10845245 |
| chr8 | 11100205 | 11100353 |
| chr8 | 11252347 | 11252477 |
| chr8 | 11297191 | 11297339 |
| chr8 | 11545814 | 11545942 |
| chr8 | 11613691 | 11614016 |
| chr8 | 11812392 | 11812538 |
| chr8 | 11906850 | 11906972 |
| chr8 | 11981016 | 11981136 |
| chr8 | 11993806 | 11993946 |
| chr8 | 12070050 | 12070174 |
| chr8 | 12153414 | 12153536 |
| chr8 | 12312571 | 12312697 |
| chr8 | 12338782 | 12338930 |
| chr8 | 12432527 | 12432651 |
| chr8 | 12435417 | 12435572 |
| chr8 | 12490642 | 12490766 |
| chr8 | 12493517 | 12493664 |
| chr8 | 12562030 | 12562158 |
| chr8 | 12695142 | 12695266 |
| chr8 | 12799972 | 12800096 |
| chr8 | 12950731 | 12950855 |
| chr8 | 13165466 | 13165590 |
| chr8 | 13294577 | 13294719 |
| chr8 | 13464288 | 13464412 |
| chr8 | 13610667 | 13610791 |
| chr8 | 13792018 | 13792148 |
| chr8 | 13971082 | 13971206 |
| chr8 | 14111597 | 14111729 |
| chr8 | 14215075 | 14215203 |
| chr8 | 14374475 | 14374601 |
| chr8 | 14483227 | 14483381 |
| chr8 | 14625213 | 14625337 |
| chr8 | 14864961 | 14865107 |
| chr8 | 14989161 | 14989313 |
| chr8 | 15171915 | 15172037 |
| chr8 | 15324462 | 15324602 |
| chr8 | 15425544 | 15425686 |
| chr8 | 15488180 | 15488316 |
| chr8 | 15743574 | 15743696 |
| chr8 | 15865467 | 15865619 |
| chr8 | 16024171 | 16024295 |
| chr8 | 16106415 | 16106539 |
| chr8 | 16364326 | 16364450 |
| chr8 | 16478704 | 16478840 |
| chr8 | 16644362 | 16644510 |
| chr8 | 16688976 | 16689132 |
| chr8 | 16933110 | 16933256 |
| chr8 | 17090886 | 17091012 |
| chr8 | 17135928 | 17136064 |
| chr8 | 17385761 | 17385887 |
| chr8 | 17444128 | 17444286 |
| chr8 | 17702292 | 17702444 |
| chr8 | 17844275 | 17844401 |
| chr8 | 17947972 | 17948094 |
| chr8 | 18083079 | 18083203 |
| chr8 | 18231652 | 18231780 |
| chr8 | 18391258 | 18391380 |
| chr8 | 18510895 | 18511019 |
| chr8 | 18740899 | 18741027 |
| chr8 | 18901031 | 18901169 |
| chr8 | 18973722 | 18973846 |
| chr8 | 19216162 | 19216316 |
| chr8 | 19347132 | 19347254 |
| chr8 | 19407296 | 19407444 |
| chr8 | 19590742 | 19590870 |
| chr8 | 19691543 | 19691671 |
| chr8 | 19865173 | 19865297 |
| chr8 | 20100989 | 20101113 |
| chr8 | 20151013 | 20151149 |
| chr8 | 20293808 | 20293948 |
| chr8 | 20529982 | 20530122 |
| chr8 | 20639452 | 20639576 |
| chr8 | 20851913 | 20852037 |
| chr8 | 20932822 | 20932950 |
| chr8 | 21093873 | 21093999 |
| chr8 | 21257157 | 21257283 |
| chr8 | 21441565 | 21441713 |
| chr8 | 21510662 | 21510788 |
| chr8 | 21740186 | 21740318 |
| chr8 | 21884466 | 21884608 |
| chr8 | 21946001 | 21946125 |
| chr8 | 22181285 | 22181429 |
| chr8 | 22282676 | 22282828 |
| chr8 | 22495930 | 22496054 |
| chr8 | 22669337 | 22669463 |
| chr8 | 22710638 | 22710774 |
| chr8 | 22916466 | 22916604 |
| chr8 | 23080011 | 23080137 |
| chr8 | 23212150 | 23212272 |
| chr8 | 23328479 | 23328617 |
| chr8 | 23438921 | 23439061 |
| chr8 | 23717159 | 23717285 |
| chr8 | 23776964 | 23777094 |
| chr8 | 23887202 | 23887327 |
| chr8 | 24105720 | 24105846 |
| chr8 | 24295050 | 24295172 |
| chr8 | 24450375 | 24450501 |
| chr8 | 24597191 | 24597369 |
| chr8 | 24641893 | 24642035 |
| chr8 | 24821563 | 24821691 |
| chr8 | 25045860 | 25045994 |
| chr8 | 25160828 | 25160962 |
| chr8 | 25265838 | 25265968 |
| chr8 | 25447914 | 25448056 |
| chr8 | 25587164 | 25587290 |
| chr8 | 25762128 | 25762254 |
| chr8 | 25875274 | 25875398 |
| chr8 | 26101523 | 26101645 |
| chr8 | 26190126 | 26190264 |
| chr8 | 26419751 | 26419885 |
| chr8 | 26480961 | 26481083 |
| chr8 | 26713653 | 26713781 |
| chr8 | 26807945 | 26808101 |
| chr8 | 26934926 | 26935048 |
| chr8 | 27060168 | 27060292 |
| chr8 | 27257709 | 27257841 |
| chr8 | 27335648 | 27335786 |
| chr8 | 27590213 | 27590373 |
| chr8 | 27702446 | 27702582 |
| chr8 | 27832092 | 27832214 |
| chr8 | 27973796 | 27973958 |
| chr8 | 28215077 | 28215223 |
| chr8 | 28324820 | 28324978 |
| chr8 | 28520593 | 28520719 |
| chr8 | 28633633 | 28633755 |
| chr8 | 28794590 | 28794714 |
| chr8 | 28894315 | 28894439 |
| chr8 | 29096771 | 29096907 |
| chr8 | 29230002 | 29230154 |
| chr8 | 29351495 | 29351631 |
| chr8 | 29540661 | 29540801 |
| chr8 | 29682579 | 29682707 |
| chr8 | 29728240 | 29728388 |
| chr8 | 29982434 | 29982606 |
| chr8 | 30045486 | 30045642 |
| chr8 | 30230283 | 30230407 |
| chr8 | 30465200 | 30465344 |
| chr8 | 30594781 | 30594905 |
| chr8 | 30659305 | 30659429 |
| chr8 | 30846835 | 30846965 |
| chr8 | 30937724 | 30937870 |
| chr8 | 31165116 | 31165258 |
| chr8 | 31295002 | 31295142 |
| chr8 | 31453840 | 31453962 |
| chr8 | 31659841 | 31659979 |
| chr8 | 31757997 | 31758139 |
| chr8 | 31945852 | 31946002 |
| chr8 | 32000935 | 32001057 |
| chr8 | 32184880 | 32185002 |
| chr8 | 32407401 | 32407539 |
| chr8 | 32537319 | 32537441 |
| chr8 | 32648456 | 32648588 |
| chr8 | 32842797 | 32842941 |
| chr8 | 32997118 | 32997244 |
| chr8 | 33148300 | 33148446 |
| chr8 | 33325017 | 33325143 |
| chr8 | 33466312 | 33466446 |
| chr8 | 33514063 | 33514211 |
| chr8 | 33702642 | 33702768 |
| chr8 | 33779878 | 33780008 |
| chr8 | 34013245 | 34013393 |
| chr8 | 34079613 | 34079737 |
| chr8 | 34325130 | 34325258 |
| chr8 | 34479174 | 34479298 |
| chr8 | 34669864 | 34669994 |
| chr8 | 34725966 | 34726225 |
| chr8 | 34899060 | 34899208 |
| chr8 | 35098477 | 35098599 |
| chr8 | 35211411 | 35211533 |
| chr8 | 35284312 | 35284434 |
| chr8 | 35432226 | 35432350 |
| chr8 | 35716366 | 35716611 |
| chr8 | 35735475 | 35735619 |
| chr8 | 35998692 | 35998816 |
| chr8 | 36044248 | 36044404 |
| chr8 | 36184516 | 36184789 |
| chr8 | 36416406 | 36416558 |
| chr8 | 36591686 | 36591931 |
| chr8 | 36642424 | 36642570 |
| chr8 | 36826616 | 36826738 |
| chr8 | 36964033 | 36964175 |
| chr8 | 37148312 | 37148442 |
| chr8 | 37246602 | 37246748 |
| chr8 | 37472347 | 37472522 |
| chr8 | 37639036 | 37639303 |
| chr8 | 37811878 | 37812004 |
| chr8 | 37919484 | 37919630 |
| chr8 | 38049532 | 38049658 |
| chr8 | 38262481 | 38262850 |
| chr8 | 38328199 | 38328368 |
| chr8 | 38558959 | 38559097 |
| chr8 | 38664088 | 38664234 |
| chr8 | 38790872 | 38791034 |
| chr8 | 38949444 | 38949590 |
| chr8 | 39175871 | 39176023 |
| chr8 | 39224932 | 39225056 |
| chr8 | 39394013 | 39394137 |
| chr8 | 39578305 | 39578441 |
| chr8 | 39661779 | 39661925 |
| chr8 | 39855639 | 39855773 |
| chr8 | 40010762 | 40010906 |
| chr8 | 40182265 | 40182389 |
| chr8 | 40276345 | 40276467 |
| chr8 | 40383037 | 40383163 |
| chr8 | 40643152 | 40643320 |
| chr8 | 40679484 | 40679610 |
| chr8 | 40893345 | 40893471 |
| chr8 | 41069482 | 41069622 |
| chr8 | 41238220 | 41238366 |
| chr8 | 41412964 | 41413088 |
| chr8 | 41510182 | 41510308 |
| chr8 | 41651400 | 41651550 |
| chr8 | 41823484 | 41823618 |
| chr8 | 41890911 | 41891047 |
| chr8 | 42031984 | 42032114 |
| chr8 | 42301726 | 42301967 |
| chr8 | 42408443 | 42408571 |
| chr8 | 42618356 | 42618504 |
| chr8 | 42767125 | 42767377 |
| chr8 | 42797863 | 42798139 |
| chr8 | 43026972 | 43027098 |
| chr8 | 43097741 | 43097938 |
| chr8 | 43350685 | 43350857 |
| chr8 | 43482168 | 43482298 |
| chr8 | 43564896 | 43565017 |
| chr8 | 43679991 | 43680113 |
| chr8 | 43730535 | 43730656 |
| chr8 | 43756168 | 43756290 |
| chr8 | 46939070 | 46939208 |
| chr8 | 47020891 | 47021041 |
| chr8 | 47218816 | 47218940 |
| chr8 | 47402473 | 47402596 |
| chr8 | 47442806 | 47442932 |
| chr8 | 47596639 | 47596785 |
| chr8 | 47852002 | 47852226 |
| chr8 | 47952842 | 47953108 |
| chr8 | 48038567 | 48038695 |
| chr8 | 48292813 | 48292935 |
| chr8 | 48453002 | 48453281 |
| chr8 | 48486192 | 48486458 |
| chr8 | 48729667 | 48729788 |
| chr8 | 48803623 | 48803745 |
| chr8 | 48987375 | 48987496 |
| chr8 | 49112793 | 49112915 |
| chr8 | 49367229 | 49367371 |
| chr8 | 49418509 | 49418639 |
| chr8 | 49563232 | 49563356 |
| chr8 | 49699777 | 49700027 |
| chr8 | 49923341 | 49923473 |
| chr8 | 50042473 | 50042615 |
| chr8 | 50222732 | 50222876 |
| chr8 | 50315067 | 50315191 |
| chr8 | 50558698 | 50558840 |
| chr8 | 50707475 | 50707629 |
| chr8 | 50772211 | 50772349 |
| chr8 | 51011048 | 51011176 |
| chr8 | 51175651 | 51175779 |
| chr8 | 51313835 | 51313959 |
| chr8 | 51412074 | 51412206 |
| chr8 | 51561676 | 51561800 |
| chr8 | 51709737 | 51709871 |
| chr8 | 51846736 | 51846880 |
| chr8 | 52033782 | 52033906 |
| chr8 | 52153996 | 52154122 |
| chr8 | 52228030 | 52228154 |
| chr8 | 52522264 | 52522428 |
| chr8 | 52659067 | 52659213 |
| chr8 | 52797036 | 52797160 |
| chr8 | 52971626 | 52971752 |
| chr8 | 53057021 | 53057143 |
| chr8 | 53182171 | 53182305 |
| chr8 | 53289811 | 53289973 |
| chr8 | 53426473 | 53426607 |
| chr8 | 53615783 | 53615921 |
| chr8 | 53851418 | 53851542 |
| chr8 | 53912547 | 53912685 |
| chr8 | 54160056 | 54160180 |
| chr8 | 54307160 | 54307284 |
| chr8 | 54330431 | 54330557 |
| chr8 | 54494473 | 54494617 |
| chr8 | 54710106 | 54710238 |
| chr8 | 54778445 | 54778721 |
| chr8 | 54962251 | 54962377 |
| chr8 | 55178418 | 55178558 |
| chr8 | 55241167 | 55241291 |
| chr8 | 55489314 | 55489436 |
| chr8 | 55555989 | 55556111 |
| chr8 | 55721230 | 55721384 |
| chr8 | 55849311 | 55849433 |
| chr8 | 56124150 | 56124286 |
| chr8 | 56183082 | 56183212 |
| chr8 | 56349487 | 56349609 |
| chr8 | 56441940 | 56442086 |
| chr8 | 56614556 | 56614680 |
| chr8 | 56836701 | 56836825 |
| chr8 | 56948683 | 56948809 |
| chr8 | 57090075 | 57090327 |
| chr8 | 57254272 | 57254416 |
| chr8 | 57438087 | 57438211 |
| chr8 | 57480751 | 57480875 |
| chr8 | 57633693 | 57633837 |
| chr8 | 57804406 | 57804532 |
| chr8 | 58036898 | 58037030 |
| chr8 | 58147065 | 58147215 |
| chr8 | 58227223 | 58227373 |
| chr8 | 58394676 | 58394800 |
| chr8 | 58562863 | 58562997 |
| chr8 | 58823216 | 58823344 |
| chr8 | 58857071 | 58857205 |
| chr8 | 59083365 | 59083487 |
| chr8 | 59156228 | 59156352 |
| chr8 | 59390488 | 59390612 |
| chr8 | 59458332 | 59458478 |
| chr8 | 59677915 | 59678049 |
| chr8 | 59759501 | 59759627 |
| chr8 | 59938985 | 59939107 |
| chr8 | 60078024 | 60078198 |
| chr8 | 60280061 | 60280191 |
| chr8 | 60383371 | 60383495 |
| chr8 | 60535460 | 60535588 |
| chr8 | 60720607 | 60720745 |
| chr8 | 60818595 | 60818719 |
| chr8 | 61008497 | 61008641 |
| chr8 | 61196723 | 61196845 |
| chr8 | 61341647 | 61341799 |
| chr8 | 61494905 | 61495039 |
| chr8 | 61598464 | 61598588 |
| chr8 | 61759029 | 61759276 |
| chr8 | 61893887 | 61894011 |
| chr8 | 62020877 | 62021011 |
| chr8 | 62156602 | 62156744 |
| chr8 | 62400622 | 62400744 |
| chr8 | 62541295 | 62541439 |
| chr8 | 62682989 | 62683129 |
| chr8 | 62843670 | 62843810 |
| chr8 | 62943757 | 62943881 |
| chr8 | 63074383 | 63074517 |
| chr8 | 63311129 | 63311253 |
| chr8 | 63436317 | 63436457 |
| chr8 | 63557123 | 63557263 |
| chr8 | 63699220 | 63699366 |
| chr8 | 63923334 | 63923462 |
| chr8 | 63968870 | 63968992 |
| chr8 | 64173881 | 64174035 |
| chr8 | 64359977 | 64360107 |
| chr8 | 64449791 | 64449937 |
| chr8 | 64567440 | 64567606 |
| chr8 | 64677269 | 64677427 |
| chr8 | 64851079 | 64851219 |
| chr8 | 65065830 | 65065954 |
| chr8 | 65133612 | 65133766 |
| chr8 | 65356719 | 65356843 |
| chr8 | 65538067 | 65538191 |
| chr8 | 65579764 | 65579896 |
| chr8 | 65828679 | 65828801 |
| chr8 | 65929902 | 65930054 |
| chr8 | 66103889 | 66104013 |
| chr8 | 66243975 | 66244099 |
| chr8 | 66412152 | 66412276 |
| chr8 | 66489663 | 66489809 |
| chr8 | 66738287 | 66738564 |
| chr8 | 66858367 | 66858511 |
| chr8 | 66926390 | 66926516 |
| chr8 | 67119179 | 67119311 |
| chr8 | 67285333 | 67285469 |
| chr8 | 67508168 | 67508289 |
| chr8 | 67666166 | 67666304 |
| chr8 | 67760774 | 67760943 |
| chr8 | 67835257 | 67835378 |
| chr8 | 68059430 | 68059608 |
| chr8 | 68262690 | 68262814 |
| chr8 | 68389051 | 68389177 |
| chr8 | 68562848 | 68562972 |
| chr8 | 68633674 | 68633818 |
| chr8 | 68800614 | 68800754 |
| chr8 | 68977335 | 68977471 |
| chr8 | 69118487 | 69118623 |
| chr8 | 69323888 | 69324016 |
| chr8 | 69464265 | 69464407 |
| chr8 | 69504038 | 69504168 |
| chr8 | 69680919 | 69681047 |
| chr8 | 69891796 | 69891930 |
| chr8 | 69957420 | 69957548 |
| chr8 | 70202012 | 70202136 |
| chr8 | 70318110 | 70318232 |
| chr8 | 70399279 | 70399401 |
| chr8 | 70573262 | 70573404 |
| chr8 | 70811964 | 70812098 |
| chr8 | 70847034 | 70847162 |
| chr8 | 71089579 | 71089719 |
| chr8 | 71197483 | 71197625 |
| chr8 | 71344372 | 71344502 |
| chr8 | 71517345 | 71517483 |
| chr8 | 71600386 | 71600522 |
| chr8 | 71844603 | 71844733 |
| chr8 | 71923465 | 71923589 |
| chr8 | 72126549 | 72126721 |
| chr8 | 72233524 | 72233646 |
| chr8 | 72380576 | 72380712 |
| chr8 | 72507256 | 72507378 |
| chr8 | 72774552 | 72774694 |
| chr8 | 72884559 | 72884683 |
| chr8 | 72993557 | 72993683 |
| chr8 | 73158126 | 73158264 |
| chr8 | 73313556 | 73313684 |
| chr8 | 73430848 | 73430970 |
| chr8 | 73566379 | 73566501 |
| chr8 | 73692077 | 73692201 |
| chr8 | 73923246 | 73923390 |
| chr8 | 74116174 | 74116314 |
| chr8 | 74224968 | 74225092 |
| chr8 | 74299699 | 74299823 |
| chr8 | 74568553 | 74568683 |
| chr8 | 74619597 | 74619737 |
| chr8 | 74794781 | 74794919 |
| chr8 | 74954804 | 74954938 |
| chr8 | 75087202 | 75087346 |
| chr8 | 75235823 | 75235953 |
| chr8 | 75378228 | 75378350 |
| chr8 | 75536712 | 75536842 |
| chr8 | 75723078 | 75723323 |
| chr8 | 75864055 | 75864191 |
| chr8 | 75946613 | 75946735 |
| chr8 | 76181228 | 76181352 |
| chr8 | 76307665 | 76307787 |
| chr8 | 76508086 | 76508216 |
| chr8 | 76632898 | 76633034 |
| chr8 | 76793540 | 76793700 |
| chr8 | 76954043 | 76954167 |
| chr8 | 77120416 | 77120568 |
| chr8 | 77194011 | 77194145 |
| chr8 | 77294863 | 77294987 |
| chr8 | 77562044 | 77562186 |
| chr8 | 77674260 | 77674386 |
| chr8 | 77875846 | 77875976 |
| chr8 | 77971479 | 77971611 |
| chr8 | 78147770 | 78147916 |
| chr8 | 78265274 | 78265400 |
| chr8 | 78334363 | 78334493 |
| chr8 | 78584414 | 78584550 |
| chr8 | 78697218 | 78697352 |
| chr8 | 78912692 | 78912838 |
| chr8 | 79008530 | 79008656 |
| chr8 | 79139025 | 79139149 |
| chr8 | 79334408 | 79334554 |
| chr8 | 79485216 | 79485340 |
| chr8 | 79598377 | 79598511 |
| chr8 | 79776157 | 79776279 |
| chr8 | 79886008 | 79886144 |
| chr8 | 80089071 | 80089193 |
| chr8 | 80197160 | 80197312 |
| chr8 | 80290524 | 80290650 |
| chr8 | 80485388 | 80485510 |
| chr8 | 80701106 | 80701230 |
| chr8 | 80849992 | 80850114 |
| chr8 | 80878002 | 80878130 |
| chr8 | 81060968 | 81061096 |
| chr8 | 81276033 | 81276167 |
| chr8 | 81455343 | 81455469 |
| chr8 | 81545135 | 81545269 |
| chr8 | 81717386 | 81717627 |
| chr8 | 81803381 | 81803666 |
| chr8 | 82034752 | 82034880 |
| chr8 | 82157985 | 82158111 |
| chr8 | 82244905 | 82245035 |
| chr8 | 82405061 | 82405183 |
| chr8 | 82563026 | 82563147 |
| chr8 | 82808823 | 82808977 |
| chr8 | 82872951 | 82873085 |
| chr8 | 83033418 | 83033556 |
| chr8 | 83178753 | 83178877 |
| chr8 | 83299459 | 83299581 |
| chr8 | 83466419 | 83466545 |
| chr8 | 83585300 | 83585422 |
| chr8 | 83874056 | 83874180 |
| chr8 | 83920050 | 83920174 |
| chr8 | 84043137 | 84043285 |
| chr8 | 84312364 | 84312502 |
| chr8 | 84374466 | 84374608 |
| chr8 | 84485428 | 84485552 |
| chr8 | 84643926 | 84644048 |
| chr8 | 84866164 | 84866298 |
| chr8 | 84999226 | 84999356 |
| chr8 | 85152797 | 85152923 |
| chr8 | 85359923 | 85360047 |
| chr8 | 85398102 | 85398226 |
| chr8 | 85537941 | 85538065 |
| chr8 | 85715420 | 85715542 |
| chr8 | 85922700 | 85922826 |
| chr8 | 86052153 | 86052293 |
| chr8 | 86172559 | 86172697 |
| chr8 | 86391523 | 86391669 |
| chr8 | 86511697 | 86511823 |
| chr8 | 86554172 | 86554294 |
| chr8 | 86844729 | 86844852 |
| chr8 | 86995378 | 86995518 |
| chr8 | 87125100 | 87125240 |
| chr8 | 87251826 | 87251968 |
| chr8 | 87430415 | 87430539 |
| chr8 | 87562702 | 87562836 |
| chr8 | 87676981 | 87677129 |
| chr8 | 87814034 | 87814164 |
| chr8 | 87958126 | 87958248 |
| chr8 | 88088305 | 88088431 |
| chr8 | 88360747 | 88360891 |
| chr8 | 88506325 | 88506469 |
| chr8 | 88602313 | 88602443 |
| chr8 | 88711927 | 88712065 |
| chr8 | 88937101 | 88937231 |
| chr8 | 89120437 | 89120569 |
| chr8 | 89245537 | 89245679 |
| chr8 | 89310478 | 89310602 |
| chr8 | 89428157 | 89428297 |
| chr8 | 89721717 | 89721847 |
| chr8 | 89754016 | 89754140 |
| chr8 | 89971159 | 89971293 |
| chr8 | 90033242 | 90033386 |
| chr8 | 90292091 | 90292217 |
| chr8 | 90450125 | 90450269 |
| chr8 | 90545732 | 90545876 |
| chr8 | 90775951 | 90776073 |
| chr8 | 90877462 | 90877594 |
| chr8 | 91055839 | 91055965 |
| chr8 | 91082006 | 91082148 |
| chr8 | 91374188 | 91374322 |
| chr8 | 91415981 | 91416105 |
| chr8 | 91580619 | 91580753 |
| chr8 | 91688889 | 91689013 |
| chr8 | 91925320 | 91925444 |
| chr8 | 92038250 | 92038382 |
| chr8 | 92190977 | 92191109 |
| chr8 | 92304066 | 92304194 |
| chr8 | 92519821 | 92519968 |
| chr8 | 92691177 | 92691321 |
| chr8 | 92777351 | 92777487 |
| chr8 | 92942375 | 92942523 |
| chr8 | 93053507 | 93053629 |
| chr8 | 93222798 | 93222924 |
| chr8 | 93415561 | 93415683 |
| chr8 | 93609262 | 93609386 |
| chr8 | 93756110 | 93756272 |
| chr8 | 93850223 | 93850401 |
| chr8 | 94034844 | 94034978 |
| chr8 | 94138864 | 94138994 |
| chr8 | 94348994 | 94349118 |
| chr8 | 94516776 | 94516900 |
| chr8 | 94605659 | 94605791 |
| chr8 | 94782673 | 94782807 |
| chr8 | 94904999 | 94905143 |
| chr8 | 95011844 | 95011976 |
| chr8 | 95202267 | 95202405 |
| chr8 | 95418379 | 95418509 |
| chr8 | 95513648 | 95513780 |
| chr8 | 95621827 | 95621951 |
| chr8 | 95756287 | 95756435 |
| chr8 | 95937416 | 95937552 |
| chr8 | 96161998 | 96162240 |
| chr8 | 96242543 | 96242679 |
| chr8 | 96344387 | 96344515 |
| chr8 | 96534966 | 96535096 |
| chr8 | 96678133 | 96678257 |
| chr8 | 96922657 | 96922801 |
| chr8 | 97026290 | 97026444 |
| chr8 | 97225085 | 97225209 |
| chr8 | 97305853 | 97305991 |
| chr8 | 97482002 | 97482142 |
| chr8 | 97530314 | 97530440 |
| chr8 | 97768520 | 97768642 |
| chr8 | 97946464 | 97946604 |
| chr8 | 98050459 | 98050591 |
| chr8 | 98129068 | 98129216 |
| chr8 | 98324366 | 98324488 |
| chr8 | 98561072 | 98561343 |
| chr8 | 98625579 | 98625703 |
| chr8 | 98727772 | 98727896 |
| chr8 | 98804329 | 98804457 |
| chr8 | 98831111 | 98831233 |
| chr8 | 98867790 | 98867934 |
| chr8 | 98954194 | 98954330 |
| chr8 | 99037092 | 99037224 |
| chr8 | 99045940 | 99046066 |
| chr8 | 99125852 | 99125976 |
| chr8 | 99292650 | 99292800 |
| chr8 | 99339744 | 99339868 |
| chr8 | 99554143 | 99554267 |
| chr8 | 99720952 | 99721076 |
| chr8 | 99851653 | 99851803 |
| chr8 | 99939863 | 99939987 |
| chr8 | 100167593 | 100167739 |
| chr8 | 100332122 | 100332246 |
| chr8 | 100512426 | 100512552 |
| chr8 | 100609239 | 100609375 |
| chr8 | 100799798 | 100799930 |
| chr8 | 100940578 | 100940699 |
| chr8 | 100997683 | 100997809 |
| chr8 | 101167359 | 101167483 |
| chr8 | 101381849 | 101381977 |
| chr8 | 101575149 | 101575299 |
| chr8 | 101726330 | 101726458 |
| chr8 | 101841498 | 101841634 |
| chr8 | 102024726 | 102024862 |
| chr8 | 102057543 | 102057679 |
| chr8 | 102188072 | 102188212 |
| chr8 | 102357414 | 102357678 |
| chr8 | 102484434 | 102484558 |
| chr8 | 102753516 | 102753664 |
| chr8 | 102866334 | 102866458 |
| chr8 | 102971511 | 102971635 |
| chr8 | 103194836 | 103194970 |
| chr8 | 103268713 | 103268839 |
| chr8 | 103504303 | 103504451 |
| chr8 | 103622458 | 103622582 |
| chr8 | 103811032 | 103811174 |
| chr8 | 103889767 | 103889889 |
| chr8 | 104003162 | 104003306 |
| chr8 | 104138526 | 104138656 |
| chr8 | 104306641 | 104306771 |
| chr8 | 104529374 | 104529514 |
| chr8 | 104626996 | 104627120 |
| chr8 | 104853320 | 104853466 |
| chr8 | 104956645 | 104956785 |
| chr8 | 105153734 | 105153866 |
| chr8 | 105185547 | 105185669 |
| chr8 | 105351936 | 105352086 |
| chr8 | 105584473 | 105584625 |
| chr8 | 105664212 | 105664360 |
| chr8 | 105851314 | 105851438 |
| chr8 | 105948078 | 105948224 |
| chr8 | 106199618 | 106199762 |
| chr8 | 106344105 | 106344229 |
| chr8 | 106474356 | 106474489 |
| chr8 | 106641807 | 106642061 |
| chr8 | 106746383 | 106746507 |
| chr8 | 106965461 | 106965585 |
| chr8 | 107014476 | 107014612 |
| chr8 | 107149350 | 107149485 |
| chr8 | 107326848 | 107326976 |
| chr8 | 107429820 | 107429962 |
| chr8 | 107698884 | 107699030 |
| chr8 | 107772337 | 107772477 |
| chr8 | 107924059 | 107924187 |
| chr8 | 108068225 | 108068369 |
| chr8 | 108192566 | 108192690 |
| chr8 | 108372868 | 108372992 |
| chr8 | 108548708 | 108548858 |
| chr8 | 108644400 | 108644536 |
| chr8 | 108879937 | 108880061 |
| chr8 | 108962162 | 108962308 |
| chr8 | 109090084 | 109090210 |
| chr8 | 109307518 | 109307640 |
| chr8 | 109500979 | 109501107 |
| chr8 | 109552132 | 109552256 |
| chr8 | 109706035 | 109706163 |
| chr8 | 109918014 | 109918160 |
| chr8 | 110033442 | 110033576 |
| chr8 | 110130019 | 110130155 |
| chr8 | 110310404 | 110310536 |
| chr8 | 110475949 | 110476073 |
| chr8 | 110670249 | 110670373 |
| chr8 | 110733129 | 110733257 |
| chr8 | 110946420 | 110946572 |
| chr8 | 111142794 | 111142916 |
| chr8 | 111298485 | 111298615 |
| chr8 | 111407900 | 111408050 |
| chr8 | 111614909 | 111615051 |
| chr8 | 111689837 | 111690106 |
| chr8 | 111805831 | 111805963 |
| chr8 | 111977876 | 111978014 |
| chr8 | 112121009 | 112121131 |
| chr8 | 112364496 | 112364650 |
| chr8 | 112515182 | 112515308 |
| chr8 | 112579713 | 112579841 |
| chr8 | 112786302 | 112786424 |
| chr8 | 112930528 | 112930684 |
| chr8 | 112987827 | 112987951 |
| chr8 | 113163914 | 113164052 |
| chr8 | 113405846 | 113405968 |
| chr8 | 113534576 | 113534728 |
| chr8 | 113695571 | 113695695 |
| chr8 | 113866934 | 113867058 |
| chr8 | 113928865 | 113928989 |
| chr8 | 114062869 | 114062991 |
| chr8 | 114303759 | 114303889 |
| chr8 | 114379863 | 114379997 |
| chr8 | 114509104 | 114509260 |
| chr8 | 114775967 | 114776095 |
| chr8 | 114799172 | 114799310 |
| chr8 | 115068789 | 115068931 |
| chr8 | 115119228 | 115119372 |
| chr8 | 115262113 | 115262251 |
| chr8 | 115477558 | 115477688 |
| chr8 | 115539660 | 115539800 |
| chr8 | 115784531 | 115784655 |
| chr8 | 115964560 | 115964706 |
| chr8 | 116065807 | 116065943 |
| chr8 | 116256182 | 116256324 |
| chr8 | 116281333 | 116281467 |
| chr8 | 116520933 | 116521137 |
| chr8 | 116698979 | 116699127 |
| chr8 | 116809990 | 116810114 |
| chr8 | 116981858 | 116981988 |
| chr8 | 117106828 | 117106962 |
| chr8 | 117306771 | 117306909 |
| chr8 | 117429721 | 117429845 |
| chr8 | 117542096 | 117542232 |
| chr8 | 117687140 | 117687270 |
| chr8 | 117843405 | 117843553 |
| chr8 | 117952660 | 117952808 |
| chr8 | 118181000 | 118181158 |
| chr8 | 118273081 | 118273205 |
| chr8 | 118489653 | 118489777 |
| chr8 | 118610199 | 118610345 |
| chr8 | 118801011 | 118801605 |
| chr8 | 118915198 | 118915336 |
| chr8 | 119017885 | 119018021 |
| chr8 | 119134594 | 119134720 |
| chr8 | 119286164 | 119286286 |
| chr8 | 119432266 | 119432390 |
| chr8 | 119690937 | 119691065 |
| chr8 | 119831266 | 119831408 |
| chr8 | 119926927 | 119927065 |
| chr8 | 120102546 | 120102676 |
| chr8 | 120295702 | 120295876 |
| chr8 | 120446073 | 120446211 |
| chr8 | 120607717 | 120607861 |
| chr8 | 120645563 | 120645709 |
| chr8 | 120817974 | 120818102 |
| chr8 | 121063478 | 121063628 |
| chr8 | 121207346 | 121207480 |
| chr8 | 121329037 | 121329159 |
| chr8 | 121387185 | 121387337 |
| chr8 | 121642953 | 121643111 |
| chr8 | 121688155 | 121688277 |
| chr8 | 121966226 | 121966372 |
| chr8 | 122123308 | 122123438 |
| chr8 | 122218878 | 122219008 |
| chr8 | 122384166 | 122384306 |
| chr8 | 122526786 | 122526942 |
| chr8 | 122715973 | 122716097 |
| chr8 | 122826530 | 122826688 |
| chr8 | 122906702 | 122906852 |
| chr8 | 123062154 | 123062280 |
| chr8 | 123203556 | 123203682 |
| chr8 | 123410910 | 123411044 |
| chr8 | 123527107 | 123527239 |
| chr8 | 123679420 | 123679546 |
| chr8 | 123791196 | 123791338 |
| chr8 | 124042550 | 124042672 |
| chr8 | 124165762 | 124165904 |
| chr8 | 124363565 | 124363765 |
| chr8 | 124407788 | 124407928 |
| chr8 | 124628956 | 124629084 |
| chr8 | 124740441 | 124740579 |
| chr8 | 124929589 | 124929743 |
| chr8 | 125050210 | 125050356 |
| chr8 | 125133788 | 125133922 |
| chr8 | 125366957 | 125367081 |
| chr8 | 125455529 | 125455655 |
| chr8 | 125606004 | 125606140 |
| chr8 | 125728535 | 125728679 |
| chr8 | 125954188 | 125954312 |
| chr8 | 126160777 | 126160905 |
| chr8 | 126202062 | 126202214 |
| chr8 | 126417562 | 126417700 |
| chr8 | 126616786 | 126616932 |
| chr8 | 126741677 | 126741831 |
| chr8 | 126899400 | 126899534 |
| chr8 | 126984884 | 126985026 |
| chr8 | 127170312 | 127170434 |
| chr8 | 127369288 | 127369424 |
| chr8 | 127437392 | 127437522 |
| chr8 | 127604530 | 127604658 |
| chr8 | 127823596 | 127823732 |
| chr8 | 127927415 | 127927541 |
| chr8 | 128022887 | 128023025 |
| chr8 | 128203817 | 128203941 |
| chr8 | 128307006 | 128307136 |
| chr8 | 128475516 | 128475656 |
| chr8 | 128645634 | 128645758 |
| chr8 | 128838553 | 128838689 |
| chr8 | 128961054 | 128961176 |
| chr8 | 129102459 | 129102581 |
| chr8 | 129216537 | 129216685 |
| chr8 | 129347452 | 129347578 |
| chr8 | 129608275 | 129608399 |
| chr8 | 129681819 | 129681941 |
| chr8 | 129848063 | 129848199 |
| chr8 | 130029282 | 130029406 |
| chr8 | 130215659 | 130215791 |
| chr8 | 130305182 | 130305316 |
| chr8 | 130471292 | 130471418 |
| chr8 | 130577205 | 130577355 |
| chr8 | 130754803 | 130754941 |
| chr8 | 130932367 | 130932499 |
| chr8 | 130990643 | 130990781 |
| chr8 | 131217035 | 131217157 |
| chr8 | 131391195 | 131391329 |
| chr8 | 131444730 | 131444872 |
| chr8 | 131679456 | 131679600 |
| chr8 | 131806968 | 131807092 |
| chr8 | 131892849 | 131892983 |
| chr8 | 132070790 | 132070918 |
| chr8 | 132289555 | 132289681 |
| chr8 | 132365325 | 132365463 |
| chr8 | 132479033 | 132479157 |
| chr8 | 132654673 | 132654799 |
| chr8 | 132873142 | 132873266 |
| chr8 | 132972169 | 132972293 |
| chr8 | 133079494 | 133079626 |
| chr8 | 133314967 | 133315093 |
| chr8 | 133498473 | 133498627 |
| chr8 | 133615848 | 133615986 |
| chr8 | 133808359 | 133808499 |
| chr8 | 133841488 | 133841616 |
| chr8 | 133986704 | 133986834 |
| chr8 | 134215962 | 134216086 |
| chr8 | 134350015 | 134350141 |
| chr8 | 134506409 | 134506537 |
| chr8 | 134646362 | 134646486 |
| chr8 | 134732175 | 134732299 |
| chr8 | 134957440 | 134957570 |
| chr8 | 135164894 | 135165024 |
| chr8 | 135299559 | 135299689 |
| chr8 | 135475858 | 135475984 |
| chr8 | 135589213 | 135589337 |
| chr8 | 135712019 | 135712145 |
| chr8 | 135923612 | 135923740 |
| chr8 | 136072951 | 136073085 |
| chr8 | 136193477 | 136193601 |
| chr8 | 136318726 | 136318870 |
| chr8 | 136450166 | 136450294 |
| chr8 | 136653426 | 136653548 |
| chr8 | 136747557 | 136747679 |
| chr8 | 136895581 | 136895713 |
| chr8 | 137043926 | 137044062 |
| chr8 | 137228229 | 137228361 |
| chr8 | 137380069 | 137380191 |
| chr8 | 137571496 | 137571626 |
| chr8 | 137725515 | 137725647 |
| chr8 | 137829876 | 137830012 |
| chr8 | 137997544 | 137997670 |
| chr8 | 138081540 | 138081668 |
| chr8 | 138201694 | 138201838 |
| chr8 | 138401957 | 138402135 |
| chr8 | 138615319 | 138615441 |
| chr8 | 138650307 | 138650445 |
| chr8 | 138801377 | 138801501 |
| chr8 | 139047260 | 139047384 |
| chr8 | 139099431 | 139099577 |
| chr8 | 139278259 | 139278383 |
| chr8 | 139472261 | 139472395 |
| chr8 | 139652079 | 139652201 |
| chr8 | 139803392 | 139803530 |
| chr8 | 139889575 | 139889699 |
| chr8 | 140083573 | 140083697 |
| chr8 | 140180669 | 140180795 |
| chr8 | 140330522 | 140330646 |
| chr8 | 140495102 | 140495226 |
| chr8 | 140712877 | 140713277 |
| chr8 | 140807411 | 140807541 |
| chr8 | 140910252 | 140910396 |
| chr8 | 141083525 | 141083649 |
| chr8 | 141235658 | 141235806 |
| chr8 | 141339324 | 141339482 |
| chr8 | 141601452 | 141601578 |
| chr8 | 141689999 | 141690127 |
| chr8 | 141831947 | 141832071 |
| chr8 | 142009518 | 142009640 |
| chr8 | 142125253 | 142125381 |
| chr8 | 142279356 | 142279500 |
| chr8 | 142491645 | 142491767 |
| chr8 | 142589209 | 142589357 |
| chr8 | 142716287 | 142716409 |
| chr8 | 142828251 | 142828373 |
| chr8 | 143033974 | 143034104 |
| chr8 | 143239741 | 143239915 |
| chr8 | 143347856 | 143347994 |
| chr8 | 143567918 | 143568042 |
| chr8 | 143609094 | 143609240 |
| chr8 | 143824407 | 143824535 |
| chr8 | 144008143 | 144008269 |
| chr8 | 144081330 | 144081462 |
| chr8 | 144224547 | 144224679 |
| chr8 | 144375796 | 144375936 |
| chr8 | 144526855 | 144526993 |
| chr8 | 144624048 | 144624178 |
| chr8 | 144696985 | 144697125 |
| chr8 | 144732382 | 144732538 |
| chr8 | 144792421 | 144792551 |
| chr8 | 144866007 | 144866145 |
| chr8 | 144973812 | 144973966 |
| chr8 | 145142898 | 145143156 |
| chr8 | 145248329 | 145248470 |
| chr8 | 145522454 | 145522582 |
| chr8 | 145548931 | 145549065 |
| chr8 | 145744339 | 145744463 |
| chr8 | 145856730 | 145857007 |
| chr8 | 146070496 | 146070642 |
| chr8 | 146231052 | 146231206 |
| chr8 | 146285027 | 146285171 |
| chr9 | 11714 | 11838 |
| chr9 | 59151 | 59279 |
| chr9 | 72695 | 72819 |
| chr9 | 79399 | 79525 |
| chr9 | 135617 | 135760 |
| chr9 | 165523 | 165645 |
| chr9 | 222407 | 222529 |
| chr9 | 331414 | 331538 |
| chr9 | 608885 | 609043 |
| chr9 | 683378 | 683500 |
| chr9 | 846426 | 846550 |
| chr9 | 967465 | 967678 |
| chr9 | 1117737 | 1117861 |
| chr9 | 1232425 | 1232547 |
| chr9 | 1446369 | 1446493 |
| chr9 | 1599517 | 1599639 |
| chr9 | 1790398 | 1790538 |
| chr9 | 1950878 | 1951024 |
| chr9 | 2099239 | 2099373 |
| chr9 | 2241681 | 2241803 |
| chr9 | 2346372 | 2346512 |
| chr9 | 2478288 | 2478424 |
| chr9 | 2668234 | 2668362 |
| chr9 | 2725193 | 2725327 |
| chr9 | 2998732 | 2998878 |
| chr9 | 3034676 | 3034800 |
| chr9 | 3258374 | 3258496 |
| chr9 | 3410784 | 3410922 |
| chr9 | 3603731 | 3603861 |
| chr9 | 3650096 | 3650220 |
| chr9 | 3832364 | 3832488 |
| chr9 | 3990373 | 3990511 |
| chr9 | 4094267 | 4094391 |
| chr9 | 4340005 | 4340147 |
| chr9 | 4385927 | 4386067 |
| chr9 | 4614181 | 4614307 |
| chr9 | 4677367 | 4677491 |
| chr9 | 4957612 | 4957738 |
| chr9 | 4972330 | 4972472 |
| chr9 | 5176103 | 5176227 |
| chr9 | 5290996 | 5291120 |
| chr9 | 5452524 | 5452646 |
| chr9 | 5709494 | 5709616 |
| chr9 | 5742025 | 5742155 |
| chr9 | 5884243 | 5884369 |
| chr9 | 6128554 | 6128678 |
| chr9 | 6306828 | 6306958 |
| chr9 | 6359054 | 6359178 |
| chr9 | 6480324 | 6480464 |
| chr9 | 6665763 | 6665935 |
| chr9 | 6884119 | 6884251 |
| chr9 | 6915325 | 6915463 |
| chr9 | 7092725 | 7092847 |
| chr9 | 7356000 | 7356134 |
| chr9 | 7499134 | 7499268 |
| chr9 | 7653548 | 7653672 |
| chr9 | 7712342 | 7712468 |
| chr9 | 7907094 | 7907222 |
| chr9 | 8097244 | 8097368 |
| chr9 | 8162576 | 8162704 |
| chr9 | 8371353 | 8371475 |
| chr9 | 8508279 | 8508403 |
| chr9 | 8686359 | 8686485 |
| chr9 | 8812347 | 8812501 |
| chr9 | 8913754 | 8913886 |
| chr9 | 9025243 | 9025365 |
| chr9 | 9195363 | 9195503 |
| chr9 | 9412230 | 9412376 |
| chr9 | 9464054 | 9464178 |
| chr9 | 9621449 | 9621575 |
| chr9 | 9837540 | 9837662 |
| chr9 | 9924761 | 9924887 |
| chr9 | 10086344 | 10086472 |
| chr9 | 10276010 | 10276134 |
| chr9 | 10381755 | 10381881 |
| chr9 | 10659464 | 10659588 |
| chr9 | 10699982 | 10700106 |
| chr9 | 10897833 | 10897955 |
| chr9 | 10988408 | 10988532 |
| chr9 | 11203924 | 11204052 |
| chr9 | 11379046 | 11379176 |
| chr9 | 11469479 | 11469607 |
| chr9 | 11656669 | 11656791 |
| chr9 | 11843508 | 11843632 |
| chr9 | 11879784 | 11879910 |
| chr9 | 12075388 | 12075512 |
| chr9 | 12214533 | 12214669 |
| chr9 | 12401361 | 12401499 |
| chr9 | 12542442 | 12542566 |
| chr9 | 12735608 | 12735754 |
| chr9 | 12878045 | 12878169 |
| chr9 | 12942696 | 12942830 |
| chr9 | 13064741 | 13064881 |
| chr9 | 13301056 | 13301208 |
| chr9 | 13428695 | 13428823 |
| chr9 | 13614064 | 13614198 |
| chr9 | 13676396 | 13676536 |
| chr9 | 13937037 | 13937185 |
| chr9 | 13969158 | 13969292 |
| chr9 | 14206795 | 14206921 |
| chr9 | 14334054 | 14334184 |
| chr9 | 14456678 | 14456820 |
| chr9 | 14649794 | 14649916 |
| chr9 | 14840048 | 14840186 |
| chr9 | 14879672 | 14879794 |
| chr9 | 15013661 | 15013799 |
| chr9 | 15172749 | 15172873 |
| chr9 | 15315109 | 15315241 |
| chr9 | 15577459 | 15577597 |
| chr9 | 15639952 | 15640076 |
| chr9 | 15776594 | 15776718 |
| chr9 | 15938472 | 15938604 |
| chr9 | 16146421 | 16146551 |
| chr9 | 16292595 | 16292717 |
| chr9 | 16484812 | 16484958 |
| chr9 | 16640620 | 16640764 |
| chr9 | 16669834 | 16669966 |
| chr9 | 16946583 | 16946749 |
| chr9 | 17015640 | 17015772 |
| chr9 | 17181653 | 17181777 |
| chr9 | 17280755 | 17280911 |
| chr9 | 17438006 | 17438134 |
| chr9 | 17665224 | 17665360 |
| chr9 | 17839253 | 17839377 |
| chr9 | 17902176 | 17902301 |
| chr9 | 18128117 | 18128249 |
| chr9 | 18275872 | 18276012 |
| chr9 | 18446618 | 18446752 |
| chr9 | 18521867 | 18521993 |
| chr9 | 18682878 | 18683002 |
| chr9 | 18907796 | 18907918 |
| chr9 | 19045594 | 19045726 |
| chr9 | 19206079 | 19206211 |
| chr9 | 19246246 | 19246370 |
| chr9 | 19447377 | 19447505 |
| chr9 | 19638237 | 19638363 |
| chr9 | 19801870 | 19801996 |
| chr9 | 19875848 | 19875988 |
| chr9 | 20086780 | 20086906 |
| chr9 | 20218607 | 20218783 |
| chr9 | 20341696 | 20341822 |
| chr9 | 20497090 | 20497230 |
| chr9 | 20676364 | 20676502 |
| chr9 | 20803527 | 20803669 |
| chr9 | 20902037 | 20902161 |
| chr9 | 21056045 | 21056169 |
| chr9 | 21242085 | 21242209 |
| chr9 | 21340746 | 21340874 |
| chr9 | 21491117 | 21491257 |
| chr9 | 21675487 | 21675615 |
| chr9 | 21816135 | 21816259 |
| chr9 | 22017782 | 22017916 |
| chr9 | 22173019 | 22173145 |
| chr9 | 22338066 | 22338198 |
| chr9 | 22460013 | 22460147 |
| chr9 | 22607858 | 22607982 |
| chr9 | 22732288 | 22732412 |
| chr9 | 22894911 | 22895037 |
| chr9 | 22976135 | 22976259 |
| chr9 | 23176059 | 23176187 |
| chr9 | 23280959 | 23281081 |
| chr9 | 23416438 | 23416580 |
| chr9 | 23657654 | 23657776 |
| chr9 | 23823347 | 23823469 |
| chr9 | 23888056 | 23888180 |
| chr9 | 24098007 | 24098139 |
| chr9 | 24168546 | 24168676 |
| chr9 | 24334612 | 24334864 |
| chr9 | 24514314 | 24514450 |
| chr9 | 24658277 | 24658401 |
| chr9 | 24799804 | 24799942 |
| chr9 | 24918121 | 24918245 |
| chr9 | 25149030 | 25149178 |
| chr9 | 25349430 | 25349556 |
| chr9 | 25500084 | 25500210 |
| chr9 | 25615957 | 25616103 |
| chr9 | 25691911 | 25692049 |
| chr9 | 25913675 | 25913807 |
| chr9 | 25979020 | 25979142 |
| chr9 | 26248530 | 26248668 |
| chr9 | 26409967 | 26410091 |
| chr9 | 26555723 | 26555849 |
| chr9 | 26649380 | 26649502 |
| chr9 | 26840559 | 26840683 |
| chr9 | 26960033 | 26960173 |
| chr9 | 27041586 | 27041724 |
| chr9 | 27187138 | 27187284 |
| chr9 | 27389766 | 27389906 |
| chr9 | 27605897 | 27606031 |
| chr9 | 27723820 | 27723946 |
| chr9 | 27851939 | 27852063 |
| chr9 | 28053490 | 28053640 |
| chr9 | 28140161 | 28140289 |
| chr9 | 28340080 | 28340206 |
| chr9 | 28455106 | 28455232 |
| chr9 | 28624404 | 28624530 |
| chr9 | 28722646 | 28722794 |
| chr9 | 28956149 | 28956275 |
| chr9 | 29039038 | 29039174 |
| chr9 | 29160226 | 29160362 |
| chr9 | 29363229 | 29363353 |
| chr9 | 29474993 | 29475117 |
| chr9 | 29601857 | 29601981 |
| chr9 | 29774387 | 29774539 |
| chr9 | 29973235 | 29973357 |
| chr9 | 30094351 | 30094507 |
| chr9 | 30297943 | 30298069 |
| chr9 | 30435387 | 30435525 |
| chr9 | 30604859 | 30604987 |
| chr9 | 30677257 | 30677381 |
| chr9 | 30781880 | 30782008 |
| chr9 | 30997885 | 30998011 |
| chr9 | 31084218 | 31084370 |
| chr9 | 31307894 | 31308026 |
| chr9 | 31381264 | 31381404 |
| chr9 | 31613961 | 31614093 |
| chr9 | 31667240 | 31667362 |
| chr9 | 31843677 | 31843801 |
| chr9 | 32060979 | 32061105 |
| chr9 | 32223783 | 32223907 |
| chr9 | 32271721 | 32271843 |
| chr9 | 32437193 | 32437337 |
| chr9 | 32579815 | 32579939 |
| chr9 | 32766950 | 32767092 |
| chr9 | 32898845 | 32898989 |
| chr9 | 33157335 | 33157483 |
| chr9 | 33282328 | 33282478 |
| chr9 | 33334743 | 33334883 |
| chr9 | 33571482 | 33571622 |
| chr9 | 33664797 | 33665008 |
| chr9 | 33857713 | 33857837 |
| chr9 | 34040684 | 34040808 |
| chr9 | 34208062 | 34208188 |
| chr9 | 34324623 | 34324747 |
| chr9 | 34408787 | 34408931 |
| chr9 | 34650722 | 34650846 |
| chr9 | 34805985 | 34806119 |
| chr9 | 34957139 | 34957273 |
| chr9 | 34983938 | 34984062 |
| chr9 | 35150176 | 35150304 |
| chr9 | 35345141 | 35345291 |
| chr9 | 35532039 | 35532163 |
| chr9 | 35635593 | 35635743 |
| chr9 | 35843000 | 35843142 |
| chr9 | 35883260 | 35883384 |
| chr9 | 36023257 | 36023385 |
| chr9 | 36306771 | 36306897 |
| chr9 | 36407279 | 36407407 |
| chr9 | 36462225 | 36462349 |
| chr9 | 36754687 | 36754813 |
| chr9 | 36876688 | 36876824 |
| chr9 | 37013340 | 37013488 |
| chr9 | 37061987 | 37062127 |
| chr9 | 37213605 | 37213729 |
| chr9 | 37400929 | 37401067 |
| chr9 | 37602811 | 37602939 |
| chr9 | 37686357 | 37686487 |
| chr9 | 37889191 | 37889319 |
| chr9 | 38040057 | 38040181 |
| chr9 | 38224329 | 38224465 |
| chr9 | 38350790 | 38350936 |
| chr9 | 38471860 | 38472055 |
| chr9 | 38641513 | 38641681 |
| chr9 | 38771665 | 38771861 |
| chr9 | 42046358 | 42046488 |
| chr9 | 42052108 | 42052242 |
| chr9 | 42091067 | 42091188 |
| chr9 | 42267566 | 42267694 |
| chr9 | 42337412 | 42337542 |
| chr9 | 42427888 | 42428040 |
| chr9 | 42560219 | 42560369 |
| chr9 | 42568665 | 42568787 |
| chr9 | 42601978 | 42602118 |
| chr9 | 42670278 | 42670400 |
| chr9 | 42700233 | 42700376 |
| chr9 | 42787128 | 42787250 |
| chr9 | 42808680 | 42808801 |
| chr9 | 42844375 | 42844497 |
| chr9 | 42899797 | 42899937 |
| chr9 | 42933122 | 42933244 |
| chr9 | 42941541 | 42941661 |
| chr9 | 43073861 | 43074013 |
| chr9 | 43164293 | 43164423 |
| chr9 | 44058762 | 44058914 |
| chr9 | 44149304 | 44149434 |
| chr9 | 45362798 | 45362922 |
| chr9 | 45423919 | 45424047 |
| chr9 | 45600397 | 45600519 |
| chr9 | 45642219 | 45642353 |
| chr9 | 45647960 | 45648090 |
| chr3 | 45986649 | 45986771 |
| chr9 | 46028410 | 46028544 |
| chr9 | 46034150 | 46034280 |
| chr9 | 46615850 | 46615971 |
| chr9 | 46654800 | 46654934 |
| chr9 | 46660553 | 46660683 |
| chr9 | 66268459 | 66268581 |
| chr9 | 66276854 | 66277004 |
| chr9 | 66499962 | 66500086 |
| chr9 | 66734370 | 66734498 |
| chr9 | 66861758 | 66861879 |
| chr9 | 66938343 | 66938465 |
| chr9 | 66960595 | 66960716 |
| chr9 | 67017380 | 67017502 |
| chr9 | 67106117 | 67106239 |
| chr9 | 67208705 | 67208855 |
| chr9 | 67302881 | 67303003 |
| chr9 | 67331248 | 67331367 |
| chr9 | 67360821 | 67360943 |
| chr9 | 67873432 | 67873562 |
| chr9 | 67879183 | 67879317 |
| chr9 | 67986305 | 67986457 |
| chr9 | 68139917 | 68140039 |
| chr9 | 68192641 | 68192763 |
| chr9 | 68316184 | 68316312 |
| chr9 | 68371533 | 68371657 |
| chr9 | 68504679 | 68504805 |
| chr9 | 68505084 | 68505208 |
| chr9 | 68676899 | 68677023 |
| chr9 | 68677311 | 68677437 |
| chr9 | 68809013 | 68809133 |
| chr9 | 68815785 | 68815911 |
| chr9 | 68999972 | 69000119 |
| chr9 | 69015925 | 69016049 |
| chr9 | 69016335 | 69016461 |
| chr9 | 69133655 | 69133783 |
| chr9 | 69146961 | 69147081 |
| chr9 | 69219149 | 69219293 |
| chr9 | 69249067 | 69249189 |
| chr9 | 69261790 | 69261915 |
| chr9 | 69329855 | 69329985 |
| chr9 | 69331650 | 69331784 |
| chr9 | 69441504 | 69441656 |
| chr9 | 69567686 | 69567836 |
| chr9 | 69576131 | 69576253 |
| chr9 | 69609451 | 69609589 |
| chr9 | 69664846 | 69664968 |
| chr9 | 69721625 | 69721747 |
| chr9 | 69742280 | 69742400 |
| chr9 | 69790164 | 69790284 |
| chr9 | 69825513 | 69825635 |
| chr9 | 69831769 | 69831893 |
| chr9 | 69844900 | 69845063 |
| chr9 | 69914555 | 69914683 |
| chr9 | 70087073 | 70087195 |
| chr9 | 70108958 | 70109080 |
| chr9 | 70195671 | 70195814 |
| chr9 | 70337821 | 70337942 |
| chr9 | 70359381 | 70359503 |
| chr9 | 70446588 | 70446731 |
| chr9 | 70476564 | 70476686 |
| chr9 | 70489371 | 70489497 |
| chr9 | 70663061 | 70663189 |
| chr9 | 70732619 | 70732749 |
| chr9 | 70857519 | 70857644 |
| chr9 | 70870230 | 70870352 |
| chr9 | 70900173 | 70900317 |
| chr9 | 70965878 | 70966004 |
| chr9 | 70972635 | 70972759 |
| chr9 | 71243750 | 71243888 |
| chr9 | 71325818 | 71325990 |
| chr9 | 71506405 | 71506547 |
| chr9 | 71595776 | 71595898 |
| chr9 | 71822789 | 71822917 |
| chr9 | 71901617 | 71901745 |
| chr9 | 72142614 | 72142748 |
| chr9 | 72247576 | 72247702 |
| chr9 | 72322367 | 72322521 |
| chr9 | 72609361 | 72609497 |
| chr9 | 72743114 | 72743244 |
| chr9 | 72861962 | 72862086 |
| chr9 | 72918855 | 72918979 |
| chr9 | 73166506 | 73166654 |
| chr9 | 73218804 | 73218950 |
| chr9 | 73393617 | 73393741 |
| chr9 | 73540221 | 73540353 |
| chr9 | 73706701 | 73706823 |
| chr9 | 73863570 | 73863718 |
| chr9 | 73979398 | 73979538 |
| chr9 | 74192518 | 74192640 |
| chr9 | 74397569 | 74397827 |
| chr9 | 74484597 | 74484862 |
| chr9 | 74564557 | 74564827 |
| chr9 | 74779320 | 74779444 |
| chr9 | 74897229 | 74897359 |
| chr9 | 75044519 | 75044679 |
| chr9 | 75162373 | 75162495 |
| chr9 | 75422890 | 75423154 |
| chr9 | 75478123 | 75478370 |
| chr9 | 75667164 | 75667288 |
| chr9 | 75895613 | 75895761 |
| chr9 | 75974341 | 75974475 |
| chr9 | 76065422 | 76065544 |
| chr9 | 76343253 | 76343377 |
| chr9 | 76412566 | 76412698 |
| chr9 | 76573925 | 76574059 |
| chr9 | 76775429 | 76775571 |
| chr9 | 76921287 | 76921423 |
| chr9 | 77012868 | 77013010 |
| chr9 | 77154413 | 77154537 |
| chr9 | 77361296 | 77361418 |
| chr9 | 77536717 | 77536841 |
| chr9 | 77650608 | 77650748 |
| chr9 | 77728099 | 77728243 |
| chr9 | 77904821 | 77904969 |
| chr9 | 78108723 | 78108865 |
| chr9 | 78163198 | 78163322 |
| chr9 | 78388638 | 78388782 |
| chr9 | 78528781 | 78528917 |
| chr9 | 78659497 | 78659621 |
| chr9 | 78810290 | 78810432 |
| chr9 | 78971167 | 78971293 |
| chr9 | 79089705 | 79089853 |
| chr9 | 79212850 | 79212996 |
| chr9 | 79412820 | 79412972 |
| chr9 | 79623356 | 79623486 |
| chr9 | 79763556 | 79763686 |
| chr9 | 79853598 | 79853744 |
| chr9 | 80065998 | 80066142 |
| chr9 | 80250246 | 80250382 |
| chr9 | 80271207 | 80271343 |
| chr9 | 80515306 | 80515462 |
| chr9 | 80589708 | 80589834 |
| chr9 | 80848987 | 80849159 |
| chr9 | 80918249 | 80918381 |
| chr9 | 81023630 | 81023754 |
| chr9 | 81281835 | 81281969 |
| chr9 | 81393064 | 81393188 |
| chr9 | 81534729 | 81534893 |
| chr9 | 81622219 | 81622357 |
| chr9 | 81860827 | 81860949 |
| chr9 | 81986993 | 81987141 |
| chr9 | 82069275 | 82069403 |
| chr9 | 82258213 | 82258353 |
| chr9 | 82433779 | 82433905 |
| chr9 | 82519160 | 82519282 |
| chr9 | 82707561 | 82707682 |
| chr9 | 82890903 | 82891027 |
| chr9 | 82983765 | 82983903 |
| chr9 | 83157815 | 83157943 |
| chr9 | 83302542 | 83302688 |
| chr9 | 83460229 | 83460373 |
| chr9 | 83607299 | 83607445 |
| chr9 | 83734455 | 83734581 |
| chr9 | 83950486 | 83950628 |
| chr9 | 84055079 | 84055227 |
| chr9 | 84237442 | 84237578 |
| chr9 | 84384037 | 84384193 |
| chr9 | 84492283 | 84492407 |
| chr9 | 84625727 | 84625877 |
| chr9 | 84877605 | 84877731 |
| chr9 | 85058991 | 85059135 |
| chr9 | 85169256 | 85169400 |
| chr9 | 85288741 | 85288869 |
| chr9 | 85450528 | 85450664 |
| chr9 | 85647042 | 85647188 |
| chr9 | 85781922 | 85782046 |
| chr9 | 85876653 | 85876777 |
| chr9 | 86093686 | 86093812 |
| chr9 | 86252100 | 86252222 |
| chr9 | 86330870 | 86330994 |
| chr9 | 86473510 | 86473675 |
| chr9 | 86700537 | 86700681 |
| chr9 | 86826798 | 86826920 |
| chr9 | 86899810 | 86899932 |
| chr9 | 87092737 | 87092861 |
| chr9 | 87213327 | 87213457 |
| chr9 | 87334016 | 87334138 |
| chr9 | 87559619 | 87559743 |
| chr9 | 87712192 | 87712342 |
| chr9 | 87875541 | 87875671 |
| chr9 | 87945828 | 87945960 |
| chr9 | 88178073 | 88178221 |
| chr9 | 88324703 | 88324835 |
| chr9 | 88380235 | 88380375 |
| chr9 | 88570661 | 88570793 |
| chr9 | 88800658 | 88800786 |
| chr9 | 88855087 | 88855235 |
| chr9 | 89061614 | 89061740 |
| chr9 | 89175793 | 89175917 |
| chr9 | 89310909 | 89311033 |
| chr9 | 89521664 | 89521792 |
| chr9 | 89646076 | 89646214 |
| chr9 | 89854557 | 89854683 |
| chr9 | 89911876 | 89912022 |
| chr9 | 90025477 | 90025599 |
| chr9 | 90311519 | 90311645 |
| chr9 | 90355754 | 90355898 |
| chr9 | 90474190 | 90474362 |
| chr9 | 90640692 | 90640816 |
| chr9 | 90809154 | 90809286 |
| chr9 | 91014862 | 91015000 |
| chr9 | 91153469 | 91153615 |
| chr9 | 91314080 | 91314206 |
| chr9 | 91429457 | 91429581 |
| chr9 | 91520788 | 91520936 |
| chr9 | 91681492 | 91681620 |
| chr9 | 91860246 | 91860382 |
| chr9 | 91963390 | 91963532 |
| chr9 | 92249548 | 92249672 |
| chr9 | 92340287 | 92340409 |
| chr9 | 92524032 | 92524236 |
| chr9 | 92681185 | 92681323 |
| chr9 | 92802059 | 92802189 |
| chr9 | 93000932 | 93001080 |
| chr9 | 93074152 | 93074278 |
| chr9 | 93259997 | 93260139 |
| chr9 | 93350830 | 93350954 |
| chr9 | 93593386 | 93593540 |
| chr9 | 93689207 | 93689335 |
| chr9 | 93767712 | 93767846 |
| chr9 | 93997837 | 93997961 |
| chr9 | 94139827 | 94139959 |
| chr9 | 94259438 | 94259562 |
| chr9 | 94508024 | 94508158 |
| chr9 | 94539860 | 94539986 |
| chr9 | 94783510 | 94783634 |
| chr9 | 94926756 | 94926897 |
| chr9 | 95006355 | 95006485 |
| chr9 | 95146677 | 95146821 |
| chr9 | 95337436 | 95337566 |
| chr9 | 95454030 | 95454180 |
| chr9 | 95669297 | 95669433 |
| chr9 | 95788571 | 95788709 |
| chr9 | 95908754 | 95908912 |
| chr9 | 96145764 | 96145886 |
| chr9 | 96290839 | 96290969 |
| chr9 | 96341148 | 96341274 |
| chr9 | 96543325 | 96543453 |
| chr9 | 96757778 | 96757936 |
| chr9 | 96873105 | 96873231 |
| chr9 | 96916480 | 96916622 |
| chr9 | 97150609 | 97150731 |
| chr9 | 97298969 | 97299107 |
| chr9 | 97429082 | 97429248 |
| chr9 | 97617035 | 97617181 |
| chr9 | 97789017 | 97789165 |
| chr9 | 97814716 | 97814838 |
| chr9 | 97962724 | 97962848 |
| chr9 | 98195651 | 98196045 |
| chr9 | 98393868 | 98394004 |
| chr9 | 98538864 | 98538988 |
| chr9 | 98677394 | 98677520 |
| chr9 | 98750580 | 98750710 |
| chr9 | 98971924 | 98972080 |
| chr9 | 99061800 | 99061936 |
| chr9 | 99288895 | 99289033 |
| chr9 | 99346534 | 99346658 |
| chr9 | 99504202 | 99504460 |
| chr9 | 99615071 | 99615259 |
| chr9 | 99761944 | 99762066 |
| chr9 | 99794012 | 99794133 |
| chr9 | 100044060 | 100044184 |
| chr9 | 100130283 | 100130421 |
| chr9 | 100267936 | 100268060 |
| chr9 | 100421937 | 100422083 |
| chr9 | 100632516 | 100632640 |
| chr9 | 100705134 | 100705264 |
| chr9 | 100919252 | 100919406 |
| chr9 | 101027285 | 101027433 |
| chr9 | 101128514 | 101128654 |
| chr9 | 101400508 | 101400644 |
| chr9 | 101452365 | 101452513 |
| chr9 | 101630241 | 101630393 |
| chr9 | 101851167 | 101851289 |
| chr9 | 101882974 | 101883116 |
| chr9 | 102032909 | 102033031 |
| chr9 | 102162217 | 102162353 |
| chr9 | 102390931 | 102391175 |
| chr9 | 102537936 | 102538193 |
| chr9 | 102701897 | 102702021 |
| chr9 | 102892654 | 102892778 |
| chr9 | 103041077 | 103041235 |
| chr9 | 103161214 | 103161358 |
| chr9 | 103283061 | 103283189 |
| chr9 | 103461737 | 103461867 |
| chr9 | 103533910 | 103534042 |
| chr9 | 103799659 | 103799785 |
| chr9 | 103880831 | 103880961 |
| chr9 | 104027428 | 104027552 |
| chr9 | 104227249 | 104227373 |
| chr9 | 104302089 | 104302221 |
| chr9 | 104501481 | 104501605 |
| chr9 | 104618271 | 104618405 |
| chr9 | 104794139 | 104794263 |
| chr9 | 104950921 | 104951045 |
| chr9 | 105157942 | 105158070 |
| chr9 | 105291951 | 105292085 |
| chr9 | 105372653 | 105372809 |
| chr9 | 105490408 | 105490530 |
| chr9 | 105633955 | 105634103 |
| chr9 | 105883750 | 105883878 |
| chr9 | 105983601 | 105983765 |
| chr9 | 106106302 | 106106446 |
| chr9 | 106311669 | 106311795 |
| chr9 | 106416580 | 106416710 |
| chr9 | 106601616 | 106601740 |
| chr9 | 106731231 | 106731353 |
| chr9 | 106861208 | 106861330 |
| chr9 | 106974328 | 106974458 |
| chr9 | 107170924 | 107171072 |
| chr9 | 107268789 | 107268913 |
| chr9 | 107512363 | 107512501 |
| chr9 | 107650769 | 107650891 |
| chr9 | 107816727 | 107816853 |
| chr9 | 107897502 | 107897638 |
| chr9 | 108092422 | 108092629 |
| chr9 | 108293194 | 108293332 |
| chr9 | 108359467 | 108359627 |
| chr9 | 108532923 | 108533051 |
| chr9 | 108724980 | 108725116 |
| chr9 | 108798212 | 108798338 |
| chr9 | 109010725 | 109010847 |
| chr9 | 109181829 | 109181951 |
| chr9 | 109291044 | 109291180 |
| chr9 | 109393869 | 109394007 |
| chr9 | 109529282 | 109529408 |
| chr9 | 109677353 | 109677479 |
| chr9 | 109913217 | 109913361 |
| chr9 | 110056673 | 110056825 |
| chr9 | 110146964 | 110147138 |
| chr9 | 110319847 | 110319985 |
| chr9 | 110463078 | 110463202 |
| chr9 | 110565686 | 110565810 |
| chr9 | 110836194 | 110836372 |
| chr9 | 110973991 | 110974131 |
| chr9 | 111120285 | 111120437 |
| chr9 | 111247257 | 111247379 |
| chr9 | 111419777 | 111419909 |
| chr9 | 111482117 | 111482243 |
| chr9 | 111747947 | 111748089 |
| chr9 | 111823118 | 111823252 |
| chr9 | 111928100 | 111928224 |
| chr9 | 112107281 | 112107459 |
| chr9 | 112300318 | 112300448 |
| chr9 | 112388159 | 112388299 |
| chr9 | 112579123 | 112579247 |
| chr9 | 112670304 | 112670434 |
| chr9 | 112891658 | 112891782 |
| chr9 | 113043915 | 113044037 |
| chr9 | 113155143 | 113155275 |
| chr9 | 113355518 | 113355670 |
| chr9 | 113486664 | 113486788 |
| chr9 | 113643738 | 113643876 |
| chr9 | 113845158 | 113845280 |
| chr9 | 113885565 | 113885715 |
| chr9 | 114032130 | 114032268 |
| chr9 | 114249874 | 114250010 |
| chr9 | 114405383 | 114405507 |
| chr9 | 114552148 | 114552272 |
| chr9 | 114623066 | 114623204 |
| chr9 | 114764959 | 114765099 |
| chr9 | 115048747 | 115048889 |
| chr9 | 115189156 | 115189286 |
| chr9 | 115255774 | 115255897 |
| chr9 | 115471651 | 115471795 |
| chr9 | 115512009 | 115512131 |
| chr9 | 115718959 | 115719083 |
| chr9 | 115908019 | 115908141 |
| chr9 | 116083194 | 116083318 |
| chr9 | 116131424 | 116131562 |
| chr9 | 116342563 | 116342803 |
| chr9 | 116481313 | 116481435 |
| chr9 | 116620710 | 116620979 |
| chr9 | 116782286 | 116782416 |
| chr9 | 117004867 | 117005005 |
| chr9 | 117129295 | 117129421 |
| chr9 | 117287846 | 117287970 |
| chr9 | 117414271 | 117414395 |
| chr9 | 117516429 | 117516551 |
| chr9 | 117734931 | 117735085 |
| chr9 | 117879328 | 117879450 |
| chr9 | 118045363 | 118045509 |
| chr9 | 118168340 | 118168466 |
| chr9 | 118278542 | 118278666 |
| chr9 | 118425147 | 118425271 |
| chr9 | 118623344 | 118623470 |
| chr9 | 118745077 | 118745223 |
| chr9 | 118842480 | 118842618 |
| chr9 | 118983271 | 118983407 |
| chr9 | 119153500 | 119153622 |
| chr9 | 119355751 | 119355875 |
| chr9 | 119452227 | 119452369 |
| chr9 | 119659795 | 119659931 |
| chr9 | 119807254 | 119807378 |
| chr9 | 119904742 | 119904878 |
| chr9 | 120021335 | 120021461 |
| chr9 | 120299752 | 120299880 |
| chr9 | 120371484 | 120371614 |
| chr9 | 120555763 | 120555903 |
| chr9 | 120634616 | 120634766 |
| chr9 | 120902150 | 120902298 |
| chr9 | 121014804 | 121014930 |
| chr9 | 121168531 | 121168655 |
| chr9 | 121291677 | 121291829 |
| chr9 | 121505258 | 121505386 |
| chr9 | 121559930 | 121560078 |
| chr9 | 121811233 | 121811385 |
| chr9 | 121958861 | 121959005 |
| chr9 | 122075924 | 122076050 |
| chr9 | 122223952 | 122224076 |
| chr9 | 122279367 | 122279493 |
| chr9 | 122500077 | 122500207 |
| chr9 | 122649834 | 122649962 |
| chr9 | 122804232 | 122804368 |
| chr9 | 122917375 | 122917507 |
| chr9 | 123015132 | 123015264 |
| chr9 | 123207656 | 123207784 |
| chr9 | 123460679 | 123460823 |
| chr9 | 123514363 | 123514487 |
| chr9 | 123642261 | 123642399 |
| chr9 | 123844008 | 123844152 |
| chr9 | 124027622 | 124027780 |
| chr9 | 124174767 | 124174909 |
| chr9 | 124269442 | 124269570 |
| chr9 | 124500992 | 124501261 |
| chr9 | 124653381 | 124653505 |
| chr9 | 124811604 | 124811876 |
| chr9 | 124852576 | 124852700 |
| chr9 | 125043469 | 125043615 |
| chr9 | 125236136 | 125236290 |
| chr9 | 125285307 | 125285431 |
| chr9 | 125514733 | 125514857 |
| chr9 | 125606115 | 125606241 |
| chr9 | 125811032 | 125811158 |
| chr9 | 125908206 | 125908330 |
| chr9 | 126023348 | 126023607 |
| chr9 | 126303313 | 126303459 |
| chr9 | 126458505 | 126458645 |
| chr9 | 126509748 | 126509878 |
| chr9 | 126722589 | 126722721 |
| chr9 | 126818445 | 126818567 |
| chr9 | 127016840 | 127016964 |
| chr9 | 127148179 | 127148307 |
| chr9 | 127340109 | 127340233 |
| chr9 | 127458416 | 127458542 |
| chr9 | 127635328 | 127635482 |
| chr9 | 127787162 | 127787296 |
| chr9 | 127881779 | 127881933 |
| chr9 | 127979415 | 127979539 |
| chr9 | 128141304 | 128141428 |
| chr9 | 128406936 | 128407074 |
| chr9 | 128524189 | 128524325 |
| chr9 | 128689684 | 128689822 |
| chr9 | 128719773 | 128719911 |
| chr9 | 128929878 | 128930008 |
| chr9 | 129147819 | 129147957 |
| chr9 | 129186521 | 129186677 |
| chr9 | 129439958 | 129440160 |
| chr9 | 129463471 | 129463638 |
| chr9 | 129690162 | 129690286 |
| chr3 | 129789529 | 129789675 |
| chr9 | 129997711 | 129997837 |
| chr9 | 130151528 | 130151664 |
| chr9 | 130235013 | 130235141 |
| chr9 | 130372433 | 130372573 |
| chr9 | 130614876 | 130615000 |
| chr9 | 130769752 | 130769882 |
| chr9 | 130873362 | 130873502 |
| chr9 | 131080172 | 131080296 |
| chr9 | 131211485 | 131211613 |
| chr9 | 131301569 | 131301707 |
| chr9 | 131458881 | 131459002 |
| chr9 | 131564836 | 131564958 |
| chr9 | 131747661 | 131747797 |
| chr9 | 131948283 | 131948407 |
| chr9 | 132110784 | 132110912 |
| chr9 | 132269942 | 132270068 |
| chr9 | 132319173 | 132319301 |
| chr9 | 132573498 | 132573624 |
| chr9 | 132657462 | 132657590 |
| chr9 | 132863739 | 132863863 |
| chr9 | 133033355 | 133033479 |
| chr9 | 133071034 | 133071224 |
| chr9 | 133226528 | 133226664 |
| chr9 | 133473213 | 133473361 |
| chr9 | 133595181 | 133595307 |
| chr9 | 133783530 | 133783652 |
| chr9 | 133931638 | 133931762 |
| chr9 | 134093220 | 134093344 |
| chr9 | 134238593 | 134238717 |
| chr9 | 134299037 | 134299177 |
| chr9 | 134511692 | 134511816 |
| chr9 | 134572156 | 134572302 |
| chr9 | 134723939 | 134724063 |
| chr9 | 134995184 | 134995326 |
| chr9 | 135071935 | 135072063 |
| chr9 | 135288688 | 135288830 |
| chr9 | 135369951 | 135370093 |
| chr9 | 135474071 | 135474193 |
| chr9 | 135685427 | 135685559 |
| chr9 | 135766759 | 135766899 |
| chr9 | 136056113 | 136056241 |
| chr9 | 136118457 | 136118601 |
| chr9 | 136331207 | 136331329 |
| chr9 | 136459160 | 136459316 |
| chr9 | 136586227 | 136586351 |
| chr9 | 136731391 | 136731513 |
| chr9 | 136828779 | 136828913 |
| chr9 | 137004440 | 137004584 |
| chr9 | 137147029 | 137147151 |
| chr9 | 137406633 | 137406801 |
| chr9 | 137518247 | 137518371 |
| chr9 | 137633503 | 137633631 |
| chr9 | 137776151 | 137776301 |
| chr9 | 137933285 | 137933423 |
| chr9 | 138059196 | 138059324 |
| chr9 | 138217803 | 138217939 |
| chr9 | 138325346 | 138325490 |
| chr9 | 138533686 | 138533838 |
| chr9 | 138756338 | 138756460 |
| chr9 | 138865538 | 138865664 |
| chr9 | 138983360 | 138983500 |
| chr9 | 139163880 | 139164001 |
| chr9 | 139335559 | 139335687 |
| chr9 | 139428772 | 139428898 |
| chr9 | 139544387 | 139544511 |
| chr9 | 13970653 | 139706655 |
| chr9 | 139883110 | 139883236 |
| chr9 | 140070586 | 140070710 |
| chr9 | 140188137 | 140188279 |
| chr9 | 140370459 | 140370715 |
| chr9 | 140422370 | 140422504 |
| chr9 | 140642243 | 140642369 |
| chr9 | 140718794 | 140718980 |
| chr9 | 140876396 | 140876534 |
| chr9 | 141083200 | 141083323 |
| chr9 | 141129325 | 141129455 |
| chr9 | 141133455 | 141133577 |
| chr9 | 141144120 | 141144250 |
| chr10 | 136213 | 136336 |
| chr10 | 297575 | 297721 |
| chr10 | 426876 | 427000 |
| chr10 | 626738 | 626862 |
| chr10 | 756944 | 757068 |
| chr10 | 956324 | 956448 |
| chr10 | 1058777 | 1058907 |
| chr10 | 1215938 | 1216064 |
| chr10 | 1335777 | 1335933 |
| chr10 | 1471485 | 1471607 |
| chr10 | 1654624 | 1654766 |
| chr10 | 1813206 | 1813328 |
| chr10 | 1955509 | 1955647 |
| chr10 | 2073683 | 2073807 |
| chr10 | 2181593 | 2181731 |
| chr10 | 2345358 | 2345488 |
| chr10 | 2504259 | 2504427 |
| chr10 | 2638740 | 2638864 |
| chr10 | 2871040 | 2871162 |
| chr10 | 2950892 | 2951030 |
| chr10 | 3138301 | 3138427 |
| chr10 | 3322269 | 3322393 |
| chr10 | 3390501 | 3390629 |
| chr10 | 3628481 | 3628605 |
| chr10 | 3767377 | 3767529 |
| chr10 | 3846253 | 3846387 |
| chr10 | 4103356 | 4103480 |
| chr10 | 4203122 | 4203244 |
| chr10 | 4264749 | 4264873 |
| chr10 | 4479296 | 4479422 |
| chr10 | 4642084 | 4642242 |
| chr10 | 4848705 | 4848835 |
| chr10 | 4992168 | 4992290 |
| chr10 | 5025768 | 5025902 |
| chr10 | 5275810 | 5275936 |
| chr10 | 5338988 | 5339122 |
| chr10 | 5467349 | 5467483 |
| chr10 | 5744907 | 5745033 |
| chr10 | 5805782 | 5805906 |
| chr10 | 6053312 | 6053452 |
| chr10 | 6132602 | 6132730 |
| chr10 | 6309344 | 6309476 |
| chr10 | 6380591 | 6380715 |
| chr10 | 6629149 | 6629293 |
| chr10 | 6677693 | 6677815 |
| chr10 | 6937784 | 6937910 |
| chr10 | 6997159 | 6997285 |
| chr10 | 7157412 | 7157534 |
| chr10 | 7385752 | 7385874 |
| chr10 | 7480612 | 7480750 |
| chr10 | 7685565 | 7685687 |
| chr10 | 7813073 | 7813201 |
| chr10 | 7932276 | 7932420 |
| chr10 | 8108732 | 8108860 |
| chr10 | 8178478 | 8178604 |
| chr10 | 8399693 | 8399847 |
| chr10 | 8591415 | 8591557 |
| chr10 | 8694752 | 8694900 |
| chr10 | 8843574 | 8843696 |
| chr10 | 8979212 | 8979342 |
| chr10 | 9169128 | 9169296 |
| chr10 | 9273506 | 9273648 |
| chr10 | 9476802 | 9476942 |
| chr10 | 9620461 | 9620601 |
| chr10 | 9796660 | 9796784 |
| chr10 | 9954482 | 9954604 |
| chr10 | 10046767 | 10046891 |
| chr10 | 10243674 | 10243818 |
| chr10 | 10371152 | 10371280 |
| chr10 | 10456442 | 10456588 |
| chr10 | 10562907 | 10563043 |
| chr10 | 10793765 | 10793941 |
| chr10 | 10865351 | 10865489 |
| chr10 | 11016014 | 11016162 |
| chr10 | 11244514 | 11244664 |
| chr10 | 11388787 | 11388923 |
| chr10 | 11464038 | 11464186 |
| chr10 | 11693080 | 11693220 |
| chr10 | 11856105 | 11856233 |
| chr10 | 12020180 | 12020316 |
| chr10 | 12190877 | 12190999 |
| chr10 | 12227825 | 12227973 |
| chr10 | 12423668 | 12423808 |
| chr10 | 12567177 | 12567301 |
| chr10 | 12750542 | 12750684 |
| chr10 | 12842405 | 12842529 |
| chr10 | 12969665 | 12969817 |
| chr10 | 13169322 | 13169444 |
| chr10 | 13398795 | 13398925 |
| chr10 | 13495120 | 13495262 |
| chr10 | 13642006 | 13642130 |
| chr10 | 13735329 | 13735459 |
| chr10 | 13869131 | 13869265 |
| chr10 | 14014504 | 14014644 |
| chr10 | 14232090 | 14232216 |
| chr10 | 14445661 | 14445807 |
| chr10 | 14588209 | 14588331 |
| chr10 | 14652971 | 14653095 |
| chr10 | 14908185 | 14908329 |
| chr10 | 14958706 | 14958838 |
| chr10 | 15205634 | 15205764 |
| chr10 | 15340695 | 15340835 |
| chr10 | 15450875 | 15451007 |
| chr10 | 15569524 | 15569650 |
| chr10 | 15724370 | 15724496 |
| chr10 | 15934620 | 15934749 |
| chr10 | 16017957 | 16018105 |
| chr10 | 16241813 | 16241943 |
| chr10 | 16276769 | 16276923 |
| chr10 | 16477040 | 16477166 |
| chr10 | 16567244 | 16567388 |
| chr10 | 16784724 | 16784870 |
| chr10 | 16926951 | 16927089 |
| chr10 | 17135070 | 17135200 |
| chr10 | 17263054 | 17263208 |
| chr10 | 17419784 | 17419924 |
| chr10 | 17509713 | 17509843 |
| chr10 | 17742691 | 17742819 |
| chr10 | 17776559 | 17776679 |
| chr10 | 17865479 | 17865645 |
| chr10 | 17938968 | 17939092 |
| chr10 | 18112450 | 18112616 |
| chr10 | 18185885 | 18186009 |
| chr10 | 18348613 | 18348757 |
| chr10 | 18499602 | 18499724 |
| chr10 | 18548960 | 18549086 |
| chr10 | 18727835 | 18727979 |
| chr10 | 18867752 | 18867876 |
| chr10 | 19054776 | 19054904 |
| chr10 | 19197080 | 19197226 |
| chr10 | 19368330 | 19368470 |
| chr10 | 19517464 | 19517602 |
| chr10 | 19625260 | 19625410 |
| chr10 | 19722457 | 19722585 |
| chr10 | 20006293 | 20006425 |
| chr10 | 20076126 | 20076276 |
| chr10 | 20169443 | 20169567 |
| chr10 | 20326594 | 20326728 |
| chr10 | 20574249 | 20574371 |
| chr10 | 20638021 | 20638161 |
| chr10 | 20894061 | 20894195 |
| chr10 | 20994761 | 20994905 |
| chr10 | 21156189 | 21156317 |
| chr10 | 21214261 | 21214385 |
| chr10 | 21428532 | 21428702 |
| chr10 | 21646266 | 21646391 |
| chr10 | 21756240 | 21756521 |
| chr10 | 21915430 | 21915553 |
| chr10 | 22094692 | 22094853 |
| chr10 | 22250686 | 22250952 |
| chr10 | 22270711 | 22270951 |
| chr10 | 22554407 | 22554597 |
| chr10 | 22633081 | 22633375 |
| chr10 | 22792436 | 22792580 |
| chr10 | 22937708 | 22937836 |
| chr10 | 23087457 | 23087581 |
| chr10 | 23217609 | 23217761 |
| chr10 | 23437969 | 23438099 |
| chr10 | 23526584 | 23526716 |
| chr10 | 23661255 | 23661379 |
| chr10 | 23867035 | 23867314 |
| chr10 | 23927212 | 23927354 |
| chr10 | 24160196 | 24160320 |
| chr10 | 24274707 | 24274857 |
| chr10 | 24417024 | 24417286 |
| chr10 | 24614237 | 24614367 |
| chr10 | 24775322 | 24775444 |
| chr10 | 24823002 | 24823124 |
| chr10 | 25079301 | 25079445 |
| chr10 | 25155325 | 25155457 |
| chr10 | 25264965 | 25265091 |
| chr10 | 25457846 | 25457974 |
| chr10 | 25672395 | 25672527 |
| chr10 | 25782139 | 25782263 |
| chr10 | 25980082 | 25980208 |
| chr10 | 26105387 | 26105511 |
| chr10 | 26250656 | 26250782 |
| chr10 | 26411670 | 26411794 |
| chr10 | 26475966 | 26476090 |
| chr10 | 26643261 | 26643383 |
| chr10 | 26876732 | 26876890 |
| chr10 | 27025565 | 27025707 |
| chr10 | 27070515 | 27070693 |
| chr10 | 27315510 | 27315632 |
| chr10 | 27468680 | 27468814 |
| chr10 | 27574804 | 27574928 |
| chr10 | 27767967 | 27768109 |
| chr10 | 27950812 | 27950934 |
| chr10 | 28102591 | 28102727 |
| chr10 | 28171951 | 28172081 |
| chr10 | 28361854 | 28361988 |
| chr10 | 28446655 | 28446803 |
| chr10 | 28579893 | 28580029 |
| chr10 | 28840030 | 28840164 |
| chr10 | 28887136 | 28887274 |
| chr10 | 29123442 | 29123572 |
| chr10 | 29178295 | 29178417 |
| chr10 | 29338525 | 29338667 |
| chr10 | 29593298 | 29593422 |
| chr10 | 29720792 | 29720916 |
| chr10 | 29846874 | 29847026 |
| chr10 | 30030251 | 30030389 |
| chr10 | 30116366 | 30116512 |
| chr10 | 30278180 | 30278314 |
| chr10 | 30446803 | 30446941 |
| chr10 | 30529974 | 30530108 |
| chr10 | 30747510 | 30747634 |
| chr10 | 30905894 | 30906022 |
| chr10 | 31044121 | 31044251 |
| chr10 | 31233435 | 31233563 |
| chr10 | 31402713 | 31402855 |
| chr10 | 31465823 | 31465945 |
| chr10 | 31654937 | 31655063 |
| chr10 | 31780966 | 31781090 |
| chr10 | 31961782 | 31961906 |
| chr10 | 32105486 | 32105612 |
| chr10 | 32256534 | 32256658 |
| chr10 | 32401021 | 32401175 |
| chr10 | 32517782 | 32517908 |
| chr10 | 32657574 | 32657722 |
| chr10 | 32903631 | 32903765 |
| chr10 | 32951335 | 32951461 |
| chr10 | 33138850 | 33139145 |
| chr10 | 33307241 | 33307367 |
| chr10 | 33444455 | 33444589 |
| chr10 | 33595960 | 33596122 |
| chr10 | 33771708 | 33771854 |
| chr10 | 33875902 | 33876052 |
| chr10 | 33962803 | 33962925 |
| chr10 | 34241504 | 34241644 |
| chr10 | 34366549 | 34366675 |
| chr10 | 34547472 | 34547602 |
| chr10 | 34639994 | 34640122 |
| chr10 | 34831803 | 34831951 |
| chr10 | 34935090 | 34935214 |
| chr10 | 35052960 | 35053090 |
| chr10 | 35195316 | 35195452 |
| chr10 | 35445528 | 35445672 |
| chr10 | 35535623 | 35535765 |
| chr10 | 35754427 | 35754575 |
| chr10 | 35899084 | 35899222 |
| chr10 | 35923271 | 35923405 |
| chr10 | 36192053 | 36192179 |
| chr10 | 36311504 | 36311654 |
| chr10 | 36455843 | 36455967 |
| chr10 | 36516072 | 36516210 |
| chr10 | 36800666 | 36800790 |
| chr10 | 36906907 | 36907031 |
| chr10 | 37010239 | 37010385 |
| chr10 | 37256599 | 37256723 |
| chr10 | 37386346 | 37386482 |
| chr10 | 37443004 | 37443150 |
| chr10 | 37467098 | 37467244 |
| chr10 | 37478899 | 37479045 |
| chr10 | 37490663 | 37490809 |
| chr10 | 37656292 | 37656413 |
| chr10 | 37768389 | 37768515 |
| chr10 | 38009409 | 38009539 |
| chr10 | 38033708 | 38033856 |
| chr10 | 38167667 | 38167799 |
| chr10 | 38420816 | 38420950 |
| chr10 | 38695944 | 38696066 |
| chr10 | 38890672 | 38890792 |
| chr10 | 42471777 | 42471901 |
| chr10 | 42514580 | 42514702 |
| chr10 | 42602334 | 42602454 |
| chr10 | 42619798 | 42619936 |
| chr10 | 42627150 | 42627274 |
| chr10 | 42669943 | 42670063 |
| chr10 | 42682156 | 42682280 |
| chr10 | 42834427 | 42834545 |
| chr10 | 42855556 | 42855678 |
| chr10 | 43015674 | 43015796 |
| chr10 | 43226772 | 43226897 |
| chr10 | 43357153 | 43357275 |
| chr10 | 43555306 | 43555430 |
| chr10 | 43634440 | 43634785 |
| chr10 | 43718465 | 43718599 |
| chr10 | 43923692 | 43923816 |
| chr10 | 44116868 | 44116994 |
| chr10 | 44238988 | 44239134 |
| chr10 | 44456269 | 44456405 |
| chr10 | 44564312 | 44564434 |
| chr10 | 44707558 | 44707684 |
| chr10 | 44773904 | 44774030 |
| chr10 | 44920467 | 44920591 |
| chr10 | 45147631 | 45147771 |
| chr10 | 45216920 | 45217046 |
| chr10 | 45456825 | 45456951 |
| chr10 | 45644682 | 45644806 |
| chr10 | 45805649 | 45805771 |
| chr10 | 45882561 | 45882773 |
| chr10 | 46088481 | 46088645 |
| chr10 | 46195081 | 46195219 |
| chr10 | 46202782 | 46202910 |
| chr10 | 46213470 | 46213600 |
| chr10 | 46223419 | 46223541 |
| chr10 | 46249866 | 46249995 |
| chr10 | 46260234 | 46260362 |
| chr10 | 46321822 | 46321960 |
| chr10 | 46490454 | 46490580 |
| chr10 | 46588932 | 46589056 |
| chr10 | 46692522 | 46692656 |
| chr10 | 46813810 | 46813934 |
| chr10 | 46928573 | 46928697 |
| chr10 | 46971228 | 46971360 |
| chr10 | 47151120 | 47151250 |
| chr10 | 47192517 | 47192655 |
| chr10 | 47294622 | 47294746 |
| chr10 | 47410643 | 47410767 |
| chr10 | 47546812 | 47546936 |
| chr10 | 47588951 | 47589075 |
| chr10 | 47650430 | 47650576 |
| chr10 | 47695996 | 47696120 |
| chr10 | 47729335 | 47729473 |
| chr10 | 47769776 | 47769906 |
| chr10 | 47910981 | 47911110 |
| chr10 | 47921342 | 47921470 |
| chr10 | 47964747 | 47964877 |
| chr10 | 48134618 | 48134742 |
| chr10 | 48236696 | 48236834 |
| chr10 | 48278104 | 48278234 |
| chr10 | 48344169 | 48344301 |
| chr10 | 48443003 | 48443159 |
| chr10 | 48615963 | 48616107 |
| chr10 | 48677797 | 48677923 |
| chr10 | 48775813 | 48775937 |
| chr10 | 48879075 | 48879209 |
| chr10 | 48901874 | 48902012 |
| chr10 | 49003555 | 49003679 |
| chr10 | 49218654 | 49218792 |
| chr10 | 49296345 | 49296469 |
| chr10 | 49390339 | 49390460 |
| chr10 | 49550620 | 49550752 |
| chr10 | 49578403 | 49578555 |
| chr10 | 49794640 | 49794780 |
| chr10 | 49959589 | 49959715 |
| chr10 | 50042539 | 50042667 |
| chr10 | 50293372 | 50293520 |
| chr10 | 50362884 | 50363014 |
| chr10 | 50544292 | 50544430 |
| chr10 | 50733825 | 50733969 |
| chr10 | 50895328 | 50895476 |
| chr10 | 50991846 | 50991970 |
| chr10 | 51131235 | 51131357 |
| chr10 | 51225449 | 51225587 |
| chr10 | 51352889 | 51353011 |
| chr10 | 51464923 | 51465061 |
| chr10 | 51488551 | 51488671 |
| chr10 | 51517973 | 51518095 |
| chr10 | 51641747 | 51641869 |
| chr10 | 51787744 | 51787865 |
| chr10 | 51808865 | 51808993 |
| chr10 | 51819422 | 51819542 |
| chr10 | 51828419 | 51828541 |
| chr10 | 51854840 | 51854969 |
| chr10 | 51865201 | 51865329 |
| chr10 | 52094578 | 52094714 |
| chr10 | 52208948 | 52209072 |
| chr10 | 52398195 | 52398333 |
| chr10 | 52413652 | 52413773 |
| chr10 | 52446509 | 52446637 |
| chr10 | 52650428 | 52650552 |
| chr10 | 52855952 | 52856102 |
| chr10 | 52905254 | 52905394 |
| chr10 | 53051964 | 53052122 |
| chr10 | 53175127 | 53175251 |
| chr10 | 53442507 | 53442633 |
| chr10 | 53480191 | 53480313 |
| chr10 | 53646191 | 53646329 |
| chr10 | 53821164 | 53821286 |
| chr10 | 54057651 | 54057775 |
| chr10 | 54193744 | 54193870 |
| chr10 | 54299346 | 54299609 |
| chr10 | 54450986 | 54451122 |
| chr10 | 54526116 | 54526240 |
| chr10 | 54696145 | 54696269 |
| chr10 | 54854351 | 54854503 |
| chr10 | 55030359 | 55030481 |
| chr10 | 55233933 | 55234055 |
| chr10 | 55267553 | 55267683 |
| chr10 | 55516323 | 55516447 |
| chr10 | 55634173 | 55634295 |
| chr10 | 55716431 | 55716563 |
| chr10 | 55889574 | 55889706 |
| chr10 | 56032076 | 56032197 |
| chr10 | 56093502 | 56093628 |
| chr10 | 56270180 | 56270306 |
| chr10 | 56457586 | 56457710 |
| chr10 | 56536818 | 56536942 |
| chr10 | 56641361 | 56641483 |
| chr10 | 56905909 | 56906033 |
| chr10 | 57007118 | 57007270 |
| chr10 | 57097248 | 57097384 |
| chr10 | 57272191 | 57272325 |
| chr10 | 57411204 | 57411348 |
| chr10 | 57614078 | 57614200 |
| chr10 | 57787984 | 57788112 |
| chr10 | 57931425 | 57931577 |
| chr10 | 58099013 | 58099161 |
| chr10 | 58184810 | 58184932 |
| chr10 | 58388390 | 58388530 |
| chr10 | 58543635 | 58543775 |
| chr10 | 58687140 | 58687274 |
| chr10 | 58828669 | 58828803 |
| chr10 | 58967393 | 58967525 |
| chr10 | 59110235 | 59110530 |
| chr10 | 59283227 | 59283355 |
| chr10 | 59367106 | 59367254 |
| chr10 | 59515001 | 59515123 |
| chr10 | 59689146 | 59689275 |
| chr10 | 59819980 | 59820114 |
| chr10 | 59985012 | 59985180 |
| chr10 | 60125799 | 60125937 |
| chr10 | 60256878 | 60257002 |
| chr10 | 60500502 | 60500628 |
| chr10 | 60636973 | 60637109 |
| chr10 | 60766222 | 60766362 |
| chr10 | 60813062 | 60813271 |
| chr10 | 60993577 | 60993729 |
| chr10 | 61150270 | 61150398 |
| chr10 | 61350138 | 61350262 |
| chr10 | 61449450 | 61449586 |
| chr10 | 61603521 | 61603671 |
| chr10 | 61793344 | 61793476 |
| chr10 | 61924807 | 61924933 |
| chr10 | 62074228 | 62074354 |
| chr10 | 62286198 | 62286322 |
| chr10 | 62370619 | 62370743 |
| chr10 | 62554420 | 62554552 |
| chr10 | 62691621 | 62691763 |
| chr10 | 62900534 | 62900676 |
| chr10 | 62915726 | 62915874 |
| chr10 | 63155489 | 63155629 |
| chr10 | 63240212 | 63240348 |
| chr10 | 63424413 | 63424537 |
| chr10 | 63625213 | 63625359 |
| chr10 | 63785003 | 63785137 |
| chr10 | 63825739 | 63825873 |
| chr10 | 64095447 | 64095593 |
| chr10 | 64231697 | 64231825 |
| chr10 | 64277300 | 64277460 |
| chr10 | 64427595 | 64427735 |
| chr10 | 64568431 | 64568848 |
| chr10 | 64823739 | 64823883 |
| chr10 | 64916429 | 64916557 |
| chr10 | 65046988 | 65047114 |
| chr10 | 65270608 | 65270738 |
| chr10 | 65340847 | 65340969 |
| chr10 | 65563609 | 65563735 |
| chr10 | 65680432 | 65680558 |
| chr10 | 65901984 | 65902108 |
| chr10 | 65936816 | 65936948 |
| chr10 | 66124138 | 66124333 |
| chr10 | 66234302 | 66234557 |
| chr10 | 66465057 | 66465181 |
| chr10 | 66515133 | 66515259 |
| chr10 | 66751293 | 66751431 |
| chr10 | 66932103 | 66932227 |
| chr10 | 66997007 | 66997147 |
| chr10 | 67192985 | 67193113 |
| chr10 | 67302846 | 67302968 |
| chr10 | 67467408 | 67467558 |
| chr10 | 67669359 | 67669483 |
| chr10 | 67769876 | 67770042 |
| chr10 | 67950036 | 67950186 |
| chr10 | 68138304 | 68138432 |
| chr10 | 68262497 | 68262629 |
| chr10 | 68361318 | 68361468 |
| chr10 | 68485531 | 68485653 |
| chr10 | 68695159 | 68695283 |
| chr10 | 68857059 | 68857203 |
| chr10 | 69023075 | 69023197 |
| chr10 | 69113391 | 69113517 |
| chr10 | 69336035 | 69336161 |
| chr10 | 69418070 | 69418198 |
| chr10 | 69536216 | 69536384 |
| chr10 | 69665333 | 69665467 |
| chr10 | 69879585 | 69879731 |
| chr10 | 70035791 | 70035921 |
| chr10 | 70183773 | 70183919 |
| chr10 | 70383399 | 70383541 |
| chr10 | 70523878 | 70524012 |
| chr10 | 70576986 | 70577128 |
| chr10 | 70754070 | 70754210 |
| chr10 | 70912034 | 70912158 |
| chr10 | 71032052 | 71032174 |
| chr10 | 71210852 | 71210994 |
| chr10 | 71376499 | 71376623 |
| chr10 | 71484766 | 71484894 |
| chr10 | 71652540 | 71652664 |
| chr10 | 71827979 | 71828103 |
| chr10 | 72007250 | 72007378 |
| chr10 | 72166275 | 72166415 |
| chr10 | 72278026 | 72278156 |
| chr10 | 72365054 | 72365218 |
| chr10 | 72634793 | 72634945 |
| chr10 | 72729410 | 72729540 |
| chr10 | 72928071 | 72928201 |
| chr10 | 73063386 | 73063508 |
| chr10 | 73256242 | 73256392 |
| chr10 | 73402730 | 73402858 |
| chr10 | 73500077 | 73500205 |
| chr10 | 73686907 | 73687049 |
| chr10 | 73831332 | 73831456 |
| chr10 | 73883492 | 73883622 |
| chr10 | 74113245 | 74113387 |
| chr10 | 74178987 | 74179159 |
| chr10 | 74389682 | 74389818 |
| chr10 | 74594685 | 74594938 |
| chr10 | 74712492 | 74712618 |
| chr10 | 74763277 | 74763401 |
| chr10 | 74939999 | 74940129 |
| chr10 | 75160217 | 75160343 |
| chr10 | 75261533 | 75261689 |
| chr10 | 75400321 | 75400445 |
| chr10 | 75434816 | 75434954 |
| chr10 | 75524679 | 75524807 |
| chr10 | 75773302 | 75773432 |
| chr10 | 75902107 | 75902239 |
| chr10 | 76015242 | 76015364 |
| chr10 | 76180994 | 76181118 |
| chr10 | 76411265 | 76411403 |
| chr10 | 76558099 | 76558239 |
| chr10 | 76612527 | 76612651 |
| chr10 | 76760515 | 76760653 |
| chr10 | 76873353 | 76873477 |
| chr10 | 77094168 | 77094306 |
| chr10 | 77266385 | 77266549 |
| chr10 | 77436988 | 77437120 |
| chr10 | 77589798 | 77589922 |
| chr10 | 77670220 | 77670348 |
| chr10 | 77858078 | 77858210 |
| chr10 | 77930029 | 77930161 |
| chr10 | 78108910 | 78109032 |
| chr10 | 78304576 | 78304702 |
| chr10 | 78441266 | 78441388 |
| chr10 | 78602674 | 78602840 |
| chr10 | 78671576 | 78671716 |
| chr10 | 78921945 | 78922067 |
| chr10 | 78974764 | 78974888 |
| chr10 | 79246149 | 79246273 |
| chr10 | 79363758 | 79363886 |
| chr10 | 79558419 | 79558547 |
| chr10 | 79652076 | 79652204 |
| chr10 | 79829720 | 79829896 |
| chr10 | 80005172 | 80005296 |
| chr10 | 80124345 | 80124469 |
| chr10 | 80230580 | 80230710 |
| chr10 | 80335278 | 80335434 |
| chr10 | 80502407 | 80502545 |
| chr10 | 80657107 | 80657235 |
| chr10 | 80805220 | 80805344 |
| chr10 | 81000738 | 81000860 |
| chr10 | 81175900 | 81176026 |
| chr10 | 81237340 | 81237480 |
| chr10 | 81389522 | 81389788 |
| chr10 | 81392596 | 81392730 |
| chr10 | 81473378 | 81473506 |
| chr10 | 81521808 | 81521932 |
| chr10 | 81611502 | 81611630 |
| chr10 | 81715560 | 81715684 |
| chr10 | 81894444 | 81894586 |
| chr10 | 82084222 | 82084372 |
| chr10 | 82142120 | 82142252 |
| chr10 | 82344636 | 82344762 |
| chr10 | 82497647 | 82497769 |
| chr10 | 82571278 | 82571402 |
| chr10 | 82740600 | 82740730 |
| chr10 | 82968733 | 82968863 |
| chr10 | 83126021 | 83126149 |
| chr10 | 83240883 | 83241005 |
| chr10 | 83443177 | 83443307 |
| chr10 | 83520997 | 83521147 |
| chr10 | 83638326 | 83638448 |
| chr10 | 83880696 | 83880836 |
| chr10 | 84044558 | 84044680 |
| chr10 | 84156450 | 84156576 |
| chr10 | 84315707 | 84315843 |
| chr10 | 84488951 | 84489095 |
| chr10 | 84561537 | 84561677 |
| chr10 | 84742656 | 84742782 |
| chr10 | 84830545 | 84830673 |
| chr10 | 85062206 | 85062332 |
| chr10 | 85129112 | 85129252 |
| chr10 | 85401971 | 85402105 |
| chr10 | 85481617 | 85481747 |
| chr10 | 85649418 | 85649562 |
| chr10 | 85766037 | 85766159 |
| chr10 | 85905025 | 85905179 |
| chr10 | 86113452 | 86113576 |
| chr10 | 86225829 | 86225969 |
| chr10 | 86385313 | 86385437 |
| chr10 | 86502300 | 86502430 |
| chr10 | 86747640 | 86747776 |
| chr10 | 86779273 | 86779401 |
| chr10 | 87056013 | 87056141 |
| chr10 | 87104411 | 87104539 |
| chr10 | 87221311 | 87221435 |
| chr10 | 87367916 | 87368042 |
| chr10 | 87544574 | 87544835 |
| chr10 | 87734752 | 87734906 |
| chr10 | 87925719 | 87925849 |
| chr10 | 87990308 | 87990446 |
| chr10 | 88216344 | 88216474 |
| chr10 | 88336201 | 88336327 |
| chr10 | 88552687 | 88552811 |
| chr10 | 88608515 | 88608870 |
| chr10 | 88769064 | 88769202 |
| chr10 | 88809504 | 88809680 |
| chr10 | 88940614 | 88940738 |
| chr10 | 88995603 | 88995731 |
| chr10 | 89043981 | 89044105 |
| chr10 | 89127820 | 89127948 |
| chr10 | 89172248 | 89172370 |
| chr10 | 89213382 | 89213506 |
| chr10 | 89435449 | 89435579 |
| chr10 | 89504322 | 89504446 |
| chr10 | 89692006 | 89692130 |
| chr10 | 89836453 | 89836579 |
| chr10 | 89924820 | 89924952 |
| chr10 | 90160312 | 90160458 |
| chr10 | 90282986 | 90283132 |
| chr10 | 90446803 | 90446961 |
| chr10 | 90605207 | 90605333 |
| chr10 | 90661839 | 90661975 |
| chr10 | 90816407 | 90816557 |
| chr10 | 91034915 | 91035055 |
| chr10 | 91217403 | 91217527 |
| chr10 | 91302393 | 91302633 |
| chr10 | 91419570 | 91419694 |
| chr10 | 91681216 | 91681342 |
| chr10 | 91813820 | 91813946 |
| chr10 | 91970521 | 91970655 |
| chr10 | 92106819 | 92106963 |
| chr10 | 92224403 | 92224537 |
| chr10 | 92412400 | 92412544 |
| chr10 | 92533653 | 92533789 |
| chr10 | 92721052 | 92721190 |
| chr10 | 92769582 | 92769708 |
| chr10 | 92946444 | 92946586 |
| chr10 | 93176403 | 93176665 |
| chr10 | 93340008 | 93340140 |
| chr10 | 93417211 | 93417335 |
| chr10 | 93598101 | 93598243 |
| chr10 | 93701287 | 93701423 |
| chr10 | 93838255 | 93838413 |
| chr10 | 94062579 | 94062703 |
| chr10 | 94155300 | 94155424 |
| chr10 | 94409235 | 94409369 |
| chr10 | 94542524 | 94542662 |
| chr10 | 94601456 | 94601578 |
| chr10 | 94737577 | 94737711 |
| chr10 | 94863219 | 94863353 |
| chr10 | 95102327 | 95102459 |
| chr10 | 95257952 | 95258088 |
| chr10 | 95395683 | 95395813 |
| chr10 | 95563918 | 95564044 |
| chr10 | 95665209 | 95665333 |
| chr10 | 95777452 | 95777576 |
| chr10 | 95966219 | 95966359 |
| chr10 | 96191859 | 96191993 |
| chr10 | 96324061 | 96324185 |
| chr10 | 96378610 | 96378756 |
| chr10 | 96582898 | 96583019 |
| chr10 | 96732669 | 96732805 |
| chr10 | 96885244 | 96885368 |
| chr10 | 97086321 | 97086453 |
| chr10 | 97140540 | 97140676 |
| chr10 | 97278033 | 97278157 |
| chr10 | 97411663 | 97411797 |
| chr10 | 97686006 | 97686134 |
| chr10 | 97856829 | 97856969 |
| chr10 | 97885574 | 97885706 |
| chr10 | 98104856 | 98104978 |
| chr10 | 98175239 | 98175361 |
| chr10 | 98341537 | 98341659 |
| chr10 | 98595286 | 98595412 |
| chr10 | 98724578 | 98724710 |
| chr10 | 98860680 | 98860808 |
| chr10 | 98976871 | 98976997 |
| chr10 | 99122142 | 99122266 |
| chr10 | 99299939 | 99300079 |
| chr10 | 99396574 | 99396714 |
| chr10 | 99580409 | 99580533 |
| chr10 | 99736529 | 99736651 |
| chr10 | 99876043 | 99876169 |
| chr10 | 99974752 | 99974902 |
| chr10 | 100212326 | 100212452 |
| chr10 | 100318651 | 100318775 |
| chr10 | 100552782 | 100552908 |
| chr10 | 100660473 | 100660605 |
| chr10 | 100713450 | 100713596 |
| chr10 | 101002146 | 101002284 |
| chr10 | 101109208 | 101109352 |
| chr10 | 101284147 | 101284297 |
| chr10 | 101368881 | 101369003 |
| chr10 | 101509029 | 101509161 |
| chr10 | 101675613 | 101675745 |
| chr10 | 101762311 | 101762437 |
| chr10 | 102001733 | 102001859 |
| chr10 | 102132493 | 102132617 |
| chr10 | 102227812 | 102227938 |
| chr10 | 102368121 | 102368247 |
| chr10 | 102635399 | 102635523 |
| chr10 | 102692890 | 102693054 |
| chr10 | 102854618 | 102854752 |
| chr10 | 102969936 | 102970060 |
| chr10 | 103124367 | 103124489 |
| chr10 | 103392412 | 103392652 |
| chr10 | 103437837 | 103438152 |
| chr10 | 103675320 | 103675460 |
| chr10 | 103734002 | 103734126 |
| chr10 | 103882772 | 103883015 |
| chr10 | 104036375 | 104036497 |
| chr10 | 104253463 | 104253593 |
| chr10 | 104318930 | 104319054 |
| chr10 | 104568394 | 104568538 |
| chr10 | 104685405 | 104685529 |
| chr10 | 104776433 | 104776559 |
| chr10 | 105001761 | 105001885 |
| chr10 | 105138877 | 105139009 |
| chr10 | 105345932 | 105346066 |
| chr10 | 105398405 | 105398527 |
| chr10 | 105571636 | 105571780 |
| chr10 | 105758270 | 105758418 |
| chr10 | 105950545 | 105950681 |
| chr10 | 106073524 | 106073654 |
| chr10 | 106147341 | 106147479 |
| chr10 | 106357226 | 106357350 |
| chr10 | 106513539 | 106513677 |
| chr10 | 106710116 | 106710250 |
| chr10 | 106743292 | 106743420 |
| chr10 | 106930918 | 106931052 |
| chr10 | 107040831 | 107040981 |
| chr10 | 107264989 | 107265111 |
| chr10 | 107401462 | 107401716 |
| chr10 | 107593526 | 107593666 |
| chr10 | 107627600 | 107627730 |
| chr10 | 107808987 | 107809117 |
| chr10 | 108006849 | 108006973 |
| chr10 | 108095592 | 108095718 |
| chr10 | 108330912 | 108331050 |
| chr10 | 108403464 | 108403588 |
| chr10 | 108578819 | 108578943 |
| chr10 | 108676822 | 108676944 |
| chr10 | 108855344 | 108855476 |
| chr10 | 109080344 | 109080468 |
| chr10 | 109252799 | 109252951 |
| chr10 | 109314033 | 109314175 |
| chr10 | 109485094 | 109485220 |
| chr10 | 109579039 | 109579185 |
| chr10 | 109796039 | 109796173 |
| chr10 | 109984903 | 109985027 |
| chr10 | 110111461 | 110111593 |
| chr10 | 110234163 | 110234295 |
| chr10 | 110401392 | 110401530 |
| chr10 | 110513901 | 110514045 |
| chr10 | 110699014 | 110699142 |
| chr10 | 110819497 | 110819623 |
| chr10 | 111045488 | 111045614 |
| chr10 | 111094103 | 111094229 |
| chr10 | 111258076 | 111258210 |
| chr10 | 111382351 | 111382503 |
| chr10 | 111609072 | 111609228 |
| chr10 | 111800065 | 111800203 |
| chr10 | 111867024 | 111867164 |
| chr10 | 111989725 | 111989849 |
| chr10 | 112175799 | 112175957 |
| chr10 | 112299633 | 112299759 |
| chr10 | 112558834 | 112558994 |
| chr10 | 112647141 | 112647263 |
| chr10 | 112784514 | 112784638 |
| chr10 | 112954358 | 112954482 |
| chr10 | 113077420 | 113077554 |
| chr10 | 113212415 | 113212537 |
| chr10 | 113356278 | 113356414 |
| chr10 | 113571438 | 113571560 |
| chr10 | 113686309 | 113686431 |
| chr10 | 113767202 | 113767344 |
| chr10 | 113987508 | 113987636 |
| chr10 | 114094875 | 114095009 |
| chr10 | 114223192 | 114223314 |
| chr10 | 114490123 | 114490247 |
| chr10 | 114640707 | 114640831 |
| chr10 | 114771705 | 114771984 |
| chr10 | 114840551 | 114840693 |
| chr10 | 115015956 | 115016080 |
| chr10 | 115118808 | 115118954 |
| chr10 | 115302471 | 115302599 |
| chr10 | 115517042 | 115517164 |
| chr10 | 115651197 | 115651341 |
| chr10 | 115779219 | 115779347 |
| chr10 | 115972015 | 115972137 |
| chr10 | 116160271 | 116160411 |
| chr10 | 116242234 | 116242356 |
| chr10 | 116377878 | 116378002 |
| chr10 | 116472429 | 116472559 |
| chr10 | 116619526 | 116619654 |
| chr10 | 116814252 | 116814376 |
| chr10 | 116957549 | 116957673 |
| chr10 | 117163480 | 117163624 |
| chr10 | 117212424 | 117212556 |
| chr10 | 117491658 | 117491804 |
| chr10 | 117522771 | 117522895 |
| chr10 | 117753951 | 117754077 |
| chr10 | 117821288 | 117821438 |
| chr10 | 118100510 | 118100638 |
| chr10 | 118167432 | 118167564 |
| chr10 | 118328616 | 118328752 |
| chr10 | 118429049 | 118429183 |
| chr10 | 118619471 | 118619607 |
| chr10 | 118777281 | 118777405 |
| chr10 | 118916141 | 118916275 |
| chr10 | 119126256 | 119126414 |
| chr10 | 119291877 | 119292061 |
| chr10 | 119407563 | 119407685 |
| chr10 | 119474235 | 119474359 |
| chr10 | 119749049 | 119749175 |
| chr10 | 119838075 | 119838353 |
| chr10 | 119940565 | 119940705 |
| chr10 | 120189538 | 120189674 |
| chr10 | 120304096 | 120304220 |
| chr10 | 120461273 | 120461397 |
| chr10 | 120586294 | 120586420 |
| chr10 | 120676268 | 120676392 |
| chr10 | 120875714 | 120875838 |
| chr10 | 120964108 | 120964254 |
| chr10 | 121122128 | 121122256 |
| chr10 | 121315774 | 121315912 |
| chr10 | 121494643 | 121494765 |
| chr10 | 121642799 | 121642921 |
| chr10 | 121728584 | 121728712 |
| chr10 | 121969055 | 121969199 |
| chr10 | 122020561 | 122020705 |
| chr10 | 122215430 | 122215568 |
| chr10 | 122320531 | 122320679 |
| chr10 | 122483043 | 122483199 |
| chr10 | 122689874 | 122690046 |
| chr10 | 122888499 | 122888637 |
| chr10 | 122929322 | 122929448 |
| chr10 | 123180990 | 123181116 |
| chr10 | 123267565 | 123267715 |
| chr10 | 123399384 | 123399508 |
| chr10 | 123597533 | 123597657 |
| chr10 | 123698181 | 123698319 |
| chr10 | 123886833 | 123886971 |
| chr10 | 124109924 | 124110048 |
| chr10 | 124231380 | 124231530 |
| chr10 | 124322722 | 124322846 |
| chr10 | 124493721 | 124493871 |
| chr10 | 124670213 | 124670353 |
| chr10 | 124720170 | 124720294 |
| chr10 | 124881412 | 124881552 |
| chr10 | 125066909 | 125067059 |
| chr10 | 125211557 | 125211693 |
| chr10 | 125397296 | 125397420 |
| chr10 | 125492389 | 125492513 |
| chr10 | 125662913 | 125663067 |
| chr10 | 125858073 | 125858219 |
| chr10 | 125964323 | 125964463 |
| chr10 | 126156841 | 126156977 |
| chr10 | 126284899 | 126285023 |
| chr10 | 126428104 | 126428262 |
| chr10 | 126594840 | 126594962 |
| chr10 | 126743006 | 126743140 |
| chr10 | 126899604 | 126899726 |
| chr10 | 127004558 | 127004696 |
| chr10 | 127158518 | 127158668 |
| chr10 | 127288308 | 127288432 |
| chr10 | 127476812 | 127476940 |
| chr10 | 127572676 | 127572821 |
| chr10 | 127780759 | 127780897 |
| chr10 | 127988641 | 127988783 |
| chr10 | 128031368 | 128031492 |
| chr10 | 128227251 | 128227391 |
| chr10 | 128425664 | 128425796 |
| chr10 | 128543400 | 128543540 |
| chr10 | 128615558 | 128615686 |
| chr10 | 128825249 | 128825371 |
| chr10 | 129042502 | 129042632 |
| chr10 | 129117681 | 129117803 |
| chr10 | 129315518 | 129315642 |
| chr10 | 129482242 | 129482364 |
| chr10 | 129586599 | 129586737 |
| chr10 | 129692237 | 129692381 |
| chr10 | 129898830 | 129898978 |
| chr10 | 129978375 | 129978519 |
| chr10 | 130212494 | 130212620 |
| chr10 | 130295257 | 130295403 |
| chr10 | 130421137 | 130421265 |
| chr10 | 130635558 | 130635698 |
| chr10 | 130825163 | 130825309 |
| chr10 | 130895361 | 130895485 |
| chr10 | 131046833 | 131046967 |
| chr10 | 131278346 | 131278476 |
| chr10 | 131323625 | 131323773 |
| chr10 | 131497647 | 131497773 |
| chr10 | 131626614 | 131626738 |
| chr10 | 131850801 | 131850923 |
| chr10 | 132054573 | 132054703 |
| chr10 | 132144973 | 132145111 |
| chr10 | 132354594 | 132354724 |
| chr10 | 132413387 | 132413509 |
| chr10 | 132530513 | 132530637 |
| chr10 | 132794769 | 132794909 |
| chr10 | 132844331 | 132844455 |
| chr10 | 133099219 | 133099359 |
| chr10 | 133178570 | 133178694 |
| chr10 | 133309868 | 133309996 |
| chr10 | 133443456 | 133443582 |
| chr10 | 133592680 | 133592814 |
| chr10 | 133844394 | 133844518 |
| chr10 | 133939593 | 133939715 |
| chr10 | 134055012 | 134055160 |
| chr10 | 134191759 | 134191881 |
| chr10 | 134394413 | 134394578 |
| chr10 | 134553731 | 134553873 |
| chr10 | 134629005 | 134629127 |
| chr10 | 134821334 | 134821482 |
| chr10 | 134921273 | 134921395 |
| chr10 | 135175933 | 135176054 |
| chr10 | 135336178 | 135336300 |
| chr10 | 135365265 | 135365391 |
| chr11 | 158885 | 159015 |
| chr11 | 169544 | 169666 |
| chr11 | 173684 | 173814 |
| chr11 | 256712 | 256864 |
| chr11 | 443679 | 443807 |
| chr11 | 511464 | 511586 |
| chr11 | 766755 | 766877 |
| chr11 | 876679 | 876819 |
| chr11 | 942254 | 942396 |
| chr11 | 1138016 | 1138144 |
| chr11 | 1333312 | 1333454 |
| chr11 | 1498379 | 1498501 |
| chr11 | 1599592 | 1599722 |
| chr11 | 1820225 | 1820351 |
| chr11 | 1917834 | 1917960 |
| chr11 | 2140910 | 2141034 |
| chr11 | 2222751 | 2222903 |
| chr11 | 2305658 | 2305804 |
| chr11 | 2526603 | 2526735 |
| chr11 | 2622925 | 2623083 |
| chr11 | 2786240 | 2786406 |
| chr11 | 2973642 | 2973782 |
| chr11 | 3126042 | 3126166 |
| chr11 | 3196011 | 3196137 |
| chr11 | 3403110 | 3403234 |
| chr11 | 3421940 | 3422064 |
| chr11 | 3575645 | 3575797 |
| chr11 | 3577018 | 3577138 |
| chr11 | 3578476 | 3578610 |
| chr11 | 3698761 | 3698885 |
| chr11 | 3912051 | 3912175 |
| chr11 | 3986416 | 3986538 |
| chr11 | 4117623 | 4117747 |
| chr11 | 4254026 | 4254150 |
| chr11 | 4264237 | 4264361 |
| chr11 | 4350603 | 4350727 |
| chr11 | 4440772 | 4440926 |
| chr11 | 4613609 | 4613737 |
| chr11 | 4742212 | 4742358 |
| chr11 | 4905119 | 4905243 |
| chr11 | 5137790 | 5137918 |
| chr11 | 5175946 | 5176076 |
| chr11 | 5416998 | 5417122 |
| chr11 | 5584906 | 5585028 |
| chr11 | 5733731 | 5733879 |
| chr11 | 5810256 | 5810380 |
| chr11 | 6026029 | 6026193 |
| chr11 | 6063895 | 6064041 |
| chr11 | 6293128 | 6293252 |
| chr11 | 6343826 | 6343950 |
| chr11 | 6495788 | 6495918 |
| chr11 | 6766529 | 6766671 |
| chr11 | 6919378 | 6919504 |
| chr11 | 6963433 | 6963557 |
| chr11 | 7114703 | 7114843 |
| chr11 | 7387311 | 7387451 |
| chr11 | 7482188 | 7482312 |
| chr11 | 7585451 | 7585587 |
| chr11 | 7728661 | 7728785 |
| chr11 | 7930061 | 7930211 |
| chr11 | 8003249 | 8003391 |
| chr11 | 8211133 | 8211265 |
| chr11 | 8403338 | 8403480 |
| chr11 | 8522108 | 8522240 |
| chr11 | 8616203 | 8616327 |
| chr11 | 8801700 | 8801822 |
| chr1 | 8993040 | 8993172 |
| chr11 | 9145751 | 9145877 |
| chr11 | 9204332 | 9204466 |
| chr11 | 9441330 | 9441470 |
| chr11 | 9539595 | 9539719 |
| chr11 | 9791202 | 9791334 |
| chr11 | 9896047 | 9896177 |
| chr11 | 10040828 | 10040956 |
| chr11 | 10203526 | 10203654 |
| chr11 | 10372915 | 10373043 |
| chr11 | 10523453 | 10523581 |
| chr11 | 10597037 | 10597167 |
| chr11 | 10813087 | 10813209 |
| chr11 | 10915174 | 10915316 |
| chr11 | 11024510 | 11024634 |
| chr11 | 11211723 | 11211863 |
| chr11 | 11396153 | 11396277 |
| chr11 | 11549836 | 11549960 |
| chr11 | 11651498 | 11651622 |
| chr11 | 11773569 | 11773727 |
| chr11 | 11916022 | 11916156 |
| chr11 | 12182422 | 12182548 |
| chr11 | 12327218 | 12327342 |
| chr11 | 12429403 | 12429527 |
| chr11 | 12611809 | 12611935 |
| chr11 | 12745231 | 12745357 |
| chr11 | 12795370 | 12795494 |
| chr11 | 13033089 | 13033229 |
| chr11 | 13173789 | 13173921 |
| chr11 | 13281275 | 13281397 |
| chr11 | 13430491 | 13430625 |
| chr11 | 13588891 | 13589017 |
| chr11 | 13707439 | 13707563 |
| chr11 | 13848145 | 13848269 |
| chr11 | 14124225 | 14124347 |
| chr11 | 14170031 | 14170157 |
| chr11 | 14403681 | 14403815 |
| chr11 | 14477496 | 14477626 |
| chr11 | 14672894 | 14673050 |
| chr11 | 14861443 | 14861579 |
| chr11 | 14946816 | 14946944 |
| chr11 | 15062097 | 15062229 |
| chr11 | 15322866 | 15322994 |
| chr11 | 15426342 | 15426476 |
| chr11 | 15630418 | 15630542 |
| chr11 | 15696601 | 15696749 |
| chr11 | 15799339 | 15799461 |
| chr11 | 16011626 | 16011777 |
| chr11 | 16188839 | 16188965 |
| chr11 | 16306300 | 16306426 |
| chr11 | 16392170 | 16392294 |
| chr11 | 16591638 | 16591792 |
| chr11 | 16760634 | 16760782 |
| chr11 | 16937882 | 16938008 |
| chr11 | 17062923 | 17063061 |
| chr11 | 17163375 | 17163513 |
| chr11 | 17379953 | 17380095 |
| chr11 | 17446829 | 17447222 |
| chr11 | 17708495 | 17708633 |
| chr11 | 17845437 | 17845559 |
| chr11 | 17961557 | 17961681 |
| chr11 | 18169892 | 18170016 |
| chr11 | 18192284 | 18192426 |
| chr11 | 18363517 | 18363639 |
| chr11 | 18540158 | 18540296 |
| chr11 | 18657747 | 18657871 |
| chr11 | 18895982 | 18896110 |
| chr11 | 18987441 | 18987567 |
| chr11 | 19179817 | 19179965 |
| chr11 | 19339693 | 19339817 |
| chr11 | 19503332 | 19503454 |
| chr11 | 19554379 | 19554501 |
| chr11 | 19776586 | 19776722 |
| chr11 | 19864755 | 19864887 |
| chr11 | 20091881 | 20092007 |
| chr11 | 20161873 | 20161997 |
| chr11 | 20363105 | 20363247 |
| chr11 | 20547872 | 20547998 |
| chr11 | 20741016 | 20741158 |
| chr11 | 20854353 | 20854477 |
| chr11 | 21007376 | 21007532 |
| chr11 | 21124548 | 21124696 |
| chr11 | 21275105 | 21275253 |
| chr11 | 21418656 | 21418786 |
| chr11 | 21556988 | 21557128 |
| chr11 | 21757611 | 21757747 |
| chr11 | 21799674 | 21799798 |
| chr11 | 21949681 | 21949815 |
| chr11 | 22120596 | 22120738 |
| chr11 | 22352072 | 22352200 |
| chr11 | 22411917 | 22412039 |
| chr11 | 22562883 | 22563015 |
| chr11 | 22721100 | 22721228 |
| chr11 | 22954436 | 22954560 |
| chr11 | 23047636 | 23047780 |
| chr11 | 23259192 | 23259322 |
| chr11 | 23363850 | 23363980 |
| chr11 | 23447792 | 23447928 |
| chr11 | 23726519 | 23726643 |
| chr11 | 23796656 | 23796784 |
| chr11 | 23949165 | 23949291 |
| chr11 | 24134800 | 24134946 |
| chr11 | 24279643 | 24279769 |
| chr11 | 24469415 | 24469539 |
| chr11 | 24564942 | 24565090 |
| chr11 | 24724848 | 24724972 |
| chr11 | 24935470 | 24935606 |
| chr11 | 24991723 | 24991863 |
| chr11 | 25153018 | 25153156 |
| chr11 | 25309279 | 25309419 |
| chr11 | 25429546 | 25429692 |
| chr11 | 25647401 | 25647527 |
| chr11 | 25831800 | 25831932 |
| chr11 | 25875969 | 25876097 |
| chr11 | 26007293 | 26007417 |
| chr11 | 26228507 | 26228643 |
| chr11 | 26402729 | 26402855 |
| chr11 | 26471674 | 26471798 |
| chr11 | 26643356 | 26643478 |
| chr11 | 26861484 | 26861622 |
| chr11 | 26973730 | 26973868 |
| chr11 | 27087898 | 27088026 |
| chr11 | 27213826 | 27213954 |
| chr11 | 27444431 | 27444571 |
| chr11 | 27574324 | 27574468 |
| chr11 | 27729425 | 27729569 |
| chr11 | 27866731 | 27866853 |
| chr11 | 28044317 | 28044443 |
| chr11 | 28152788 | 28152926 |
| chr11 | 28264287 | 28264421 |
| chr11 | 28521917 | 28522039 |
| chr11 | 28546094 | 28546222 |
| chr11 | 28792818 | 28792942 |
| chr11 | 28989260 | 28989382 |
| chr11 | 29111063 | 29111203 |
| chr11 | 29213924 | 29214048 |
| chr11 | 29303009 | 29303133 |
| chr11 | 29464949 | 29465103 |
| chr11 | 29693488 | 29693612 |
| chr11 | 29887845 | 29887969 |
| chr11 | 29957617 | 29957763 |
| chr11 | 30187898 | 30188046 |
| chr11 | 30232527 | 30232675 |
| chr11 | 30415708 | 30415860 |
| chr11 | 30508000 | 30508122 |
| chr11 | 30676001 | 30676149 |
| chr11 | 30920340 | 30920484 |
| chr11 | 31011410 | 31011532 |
| chr11 | 31102456 | 31102582 |
| chr11 | 31286398 | 31286520 |
| chr11 | 31527544 | 31527676 |
| chr11 | 31691765 | 31691907 |
| chr11 | 31810208 | 31810348 |
| chr11 | 31949071 | 31949193 |
| chr11 | 32098667 | 32098801 |
| chr11 | 32279516 | 32279638 |
| chr11 | 32433005 | 32433129 |
| chr11 | 32474711 | 32474832 |
| chr11 | 32735858 | 32735990 |
| chr11 | 32868754 | 32868878 |
| chr11 | 32960769 | 32960890 |
| chr11 | 33111493 | 33111637 |
| chr11 | 33322492 | 33322614 |
| chr11 | 33376037 | 33376179 |
| chr11 | 33617681 | 33617805 |
| chr11 | 33660703 | 33660831 |
| chr11 | 33856391 | 33856513 |
| chr11 | 34013830 | 34013964 |
| chr11 | 34198298 | 34198422 |
| chr11 | 34354779 | 34354915 |
| chr11 | 34442907 | 34443057 |
| chr11 | 34579340 | 34579464 |
| chr11 | 34830628 | 34830752 |
| chr11 | 34935762 | 34935886 |
| chr11 | 35094417 | 35094541 |
| chr11 | 35266127 | 35266275 |
| chr11 | 35321931 | 35322053 |
| chr11 | 35446933 | 35447069 |
| chr11 | 35597082 | 35597238 |
| chr11 | 35801992 | 35802114 |
| chr11 | 35929419 | 35929573 |
| chr11 | 36055342 | 36055466 |
| chr11 | 36269304 | 36269470 |
| chr11 | 36363539 | 36363663 |
| chr11 | 36533705 | 36533833 |
| chr11 | 36767679 | 36767821 |
| chr11 | 36930105 | 36930229 |
| chr11 | 37026236 | 37026382 |
| chr11 | 37207462 | 37207594 |
| chr11 | 37245237 | 37245361 |
| chr11 | 37461534 | 37461658 |
| chr11 | 37645967 | 37646091 |
| chr11 | 37762428 | 37762552 |
| chr11 | 37967630 | 37967752 |
| chr11 | 38095061 | 38095247 |
| chr11 | 38169866 | 38170049 |
| chr11 | 38409369 | 38409505 |
| chr11 | 38447547 | 38447671 |
| chr11 | 38635503 | 38635629 |
| chr11 | 38872129 | 38872287 |
| chr11 | 38960053 | 38960175 |
| chr11 | 39065666 | 39065790 |
| chr11 | 39247956 | 39248222 |
| chr11 | 39378022 | 39378144 |
| chr11 | 39564152 | 39564286 |
| chr11 | 39755156 | 39755306 |
| chr11 | 39889022 | 39889146 |
| chr11 | 40029573 | 40029705 |
| chr11 | 40100504 | 40100654 |
| chr11 | 40248093 | 40248235 |
| chr11 | 40471364 | 40471522 |
| chr11 | 40669681 | 40669823 |
| chr11 | 40735904 | 40736028 |
| chr11 | 40875634 | 40875756 |
| chr11 | 41038909 | 41039037 |
| chr11 | 41235054 | 41235208 |
| chr11 | 41409783 | 41409905 |
| chr11 | 41532939 | 41533081 |
| chr11 | 41741408 | 41741540 |
| chr11 | 41845271 | 41845393 |
| chr11 | 41928825 | 41928961 |
| chr11 | 42138460 | 42138584 |
| chr11 | 42319701 | 42319837 |
| chr11 | 42369582 | 42369718 |
| chr11 | 42599496 | 42599632 |
| chr11 | 42760599 | 42760725 |
| chr11 | 42885101 | 42885223 |
| chr11 | 43020389 | 43020515 |
| chr11 | 43205261 | 43205403 |
| chr11 | 43244860 | 43244984 |
| chr11 | 43459354 | 43459490 |
| chr11 | 43632076 | 43632218 |
| chr11 | 43739767 | 43739905 |
| chr11 | 43872870 | 43872996 |
| chr11 | 44063738 | 44063860 |
| chr11 | 44192649 | 44192779 |
| chr11 | 44329555 | 44329926 |
| chr11 | 44444545 | 44444679 |
| chr11 | 44741112 | 44741236 |
| chr11 | 44775400 | 44775548 |
| chr11 | 44993451 | 44993595 |
| chr11 | 45042173 | 45042301 |
| chr11 | 45337277 | 45337425 |
| chr11 | 45396270 | 45396406 |
| chr11 | 45600730 | 45600858 |
| chr11 | 45730181 | 45730329 |
| chr11 | 45815932 | 45816068 |
| chr11 | 45997717 | 45997857 |
| chr11 | 46113144 | 46113266 |
| chr11 | 46304264 | 46304402 |
| chr11 | 46434169 | 46434430 |
| chr11 | 46648376 | 46648502 |
| chr11 | 46836075 | 46836203 |
| chr11 | 46990527 | 46990675 |
| chr11 | 47092996 | 47093148 |
| chr11 | 47169312 | 47169450 |
| chr11 | 47312802 | 47312940 |
| chr11 | 47477783 | 47477937 |
| chr11 | 47643821 | 47643971 |
| chr11 | 47763431 | 47763571 |
| chr11 | 47940889 | 47941011 |
| chr11 | 48056169 | 48056425 |
| chr11 | 48260895 | 48261019 |
| chr11 | 48359805 | 48360060 |
| chr11 | 48555881 | 48556005 |
| chr11 | 48665766 | 48665890 |
| chr11 | 48929780 | 48929932 |
| chr11 | 49022792 | 49022930 |
| chr11 | 49156336 | 49156458 |
| chr11 | 49208836 | 49208996 |
| chr11 | 49314880 | 49315018 |
| chr11 | 49472396 | 49472526 |
| chr11 | 49570225 | 49570353 |
| chr11 | 49660298 | 49660424 |
| chr11 | 49789614 | 49789764 |
| chr11 | 49914424 | 49914552 |
| chr11 | 50063818 | 50063942 |
| chr11 | 50174218 | 50174341 |
| chr11 | 50398637 | 50398760 |
| chr11 | 50493837 | 50493960 |
| chr11 | 50574012 | 50574152 |
| chr11 | 51240169 | 51240290 |
| chr11 | 51297377 | 51297499 |
| chr11 | 55091206 | 55091342 |
| chr11 | 55094205 | 55094349 |
| chr11 | 55356353 | 55356475 |
| chr11 | 55499109 | 55499273 |
| chr11 | 55679272 | 55679395 |
| chr11 | 55820952 | 55821092 |
| chr11 | 55895073 | 55895197 |
| chr11 | 56003053 | 56003187 |
| chr11 | 56149091 | 56149213 |
| chr11 | 56315984 | 56316124 |
| chr11 | 56590961 | 56591103 |
| chr11 | 56690626 | 56690750 |
| chr11 | 56780606 | 56780736 |
| chr11 | 56942312 | 56942452 |
| chr11 | 57081571 | 57081695 |
| chr11 | 57296585 | 57296725 |
| chr11 | 57480675 | 57480799 |
| chr11 | 57542956 | 57543086 |
| chr11 | 57696146 | 57696318 |
| chr11 | 57873045 | 57873169 |
| chr11 | 58009520 | 58009642 |
| chr11 | 58210057 | 58210183 |
| chr11 | 58312794 | 58312932 |
| chr11 | 58416766 | 58416896 |
| chr11 | 58667360 | 58667498 |
| chr11 | 58724069 | 58724193 |
| chr11 | 58891238 | 58891376 |
| chr11 | 59050936 | 59051090 |
| chr11 | 59257378 | 59257502 |
| chr11 | 59396840 | 59396962 |
| chr11 | 59483641 | 59483779 |
| chr11 | 59619795 | 59620060 |
| chr11 | 59762852 | 59762998 |
| chr11 | 59971759 | 59971883 |
| chr11 | 60174196 | 60174324 |
| chr11 | 60226600 | 60226724 |
| chr11 | 60379652 | 60379778 |
| chr11 | 60521135 | 60521259 |
| chr11 | 60765467 | 60765607 |
| chr11 | 60939387 | 60939669 |
| chr11 | 61014479 | 61014600 |
| chr11 | 61184911 | 61185177 |
| chr11 | 61271356 | 61271481 |
| chr11 | 61442749 | 61442885 |
| chr11 | 61581690 | 61581816 |
| chr11 | 61794965 | 61795091 |
| chr11 | 61889525 | 61889683 |
| chr11 | 62117619 | 62117759 |
| chr11 | 62230105 | 62230293 |
| chr11 | 62337078 | 62337320 |
| chr11 | 62480239 | 62480363 |
| chr11 | 62734163 | 62734293 |
| chr11 | 62836052 | 62836190 |
| chr11 | 63008692 | 63008820 |
| chr11 | 63130251 | 63130381 |
| chr11 | 63244622 | 63244760 |
| chr11 | 63354006 | 63354130 |
| chr11 | 63593159 | 63593291 |
| chr11 | 63718573 | 63718695 |
| chr11 | 63792887 | 63793011 |
| chr11 | 63964517 | 63964641 |
| chr11 | 64202665 | 64202817 |
| chr11 | 64352027 | 64352151 |
| chr11 | 64487306 | 64487452 |
| chr11 | 64658264 | 64658388 |
| chr11 | 64803295 | 64803425 |
| chr11 | 64924292 | 64924553 |
| chr11 | 65141368 | 65141489 |
| chr11 | 65260564 | 65260694 |
| chr11 | 65429876 | 65430024 |
| chr11 | 65531644 | 65531778 |
| chr11 | 65711465 | 65711603 |
| chr11 | 65852481 | 65852605 |
| chr11 | 65952851 | 65952995 |
| chr11 | 66116800 | 66116924 |
| chr11 | 66240131 | 66240267 |
| chr11 | 66402267 | 66402421 |
| chr11 | 66516556 | 66516683 |
| chr11 | 66719445 | 66719567 |
| chr11 | 66932438 | 66932559 |
| chr11 | 67043949 | 67044070 |
| chr11 | 67125126 | 67125368 |
| chr11 | 67324869 | 67325019 |
| chr11 | 67513977 | 67514117 |
| chr11 | 67520750 | 67520873 |
| chr11 | 67608789 | 67608911 |
| chr11 | 67699131 | 67699255 |
| chr11 | 67700616 | 67700744 |
| chr11 | 67928442 | 67928564 |
| chr11 | 68057264 | 68057398 |
| chr11 | 68216720 | 68216844 |
| chr11 | 68302027 | 68302151 |
| chr11 | 68540276 | 68540404 |
| chr11 | 68672716 | 68672888 |
| chr11 | 68785713 | 68785863 |
| chr11 | 69013965 | 69014097 |
| chr11 | 69150760 | 69150882 |
| chr11 | 69234450 | 69234582 |
| chr11 | 69354448 | 69354572 |
| chr11 | 69540683 | 69540809 |
| chr11 | 69775149 | 69775271 |
| chr11 | 69857836 | 69857962 |
| chr11 | 70044970 | 70045098 |
| chr11 | 70165167 | 70165311 |
| chr11 | 70307758 | 70307882 |
| chr11 | 70481417 | 70481565 |
| chr11 | 70680813 | 70680957 |
| chr11 | 70816913 | 70817045 |
| chr11 | 70855938 | 70856070 |
| chr11 | 71007092 | 71007220 |
| chr11 | 71147739 | 71147879 |
| chr11 | 71373681 | 71373809 |
| chr11 | 71375170 | 71375294 |
| chr11 | 71376507 | 71376659 |
| chr11 | 71543779 | 71543907 |
| chr11 | 71627709 | 71627829 |
| chr11 | 71891096 | 71891222 |
| chr11 | 71956823 | 71956949 |
| chr11 | 72130460 | 72130747 |
| chr11 | 72225735 | 72225869 |
| chr11 | 72381560 | 72381686 |
| chr11 | 72519678 | 72519804 |
| chr11 | 72691366 | 72691490 |
| chr11 | 72843414 | 72843539 |
| chr11 | 72956297 | 72956418 |
| chr11 | 73219091 | 73219223 |
| chr11 | 73248618 | 73248740 |
| chr11 | 73520631 | 73520759 |
| chr11 | 73580780 | 73580902 |
| chr11 | 73829729 | 73829869 |
| chr11 | 73916604 | 73916734 |
| chr11 | 74017231 | 74017355 |
| chr11 | 74256016 | 74256138 |
| chr11 | 74294276 | 74294404 |
| chr11 | 74553764 | 74553892 |
| chr11 | 74631616 | 74631752 |
| chr11 | 74828695 | 74828823 |
| chr11 | 74925916 | 74926044 |
| chr11 | 75142828 | 75142970 |
| chr11 | 75203609 | 75203737 |
| chr11 | 75375103 | 75375229 |
| chr11 | 75594013 | 75594136 |
| chr11 | 75647182 | 75647330 |
| chr11 | 75908057 | 75908179 |
| chr11 | 76057994 | 76058120 |
| chr11 | 76163964 | 76164090 |
| chr11 | 76329292 | 76329426 |
| chr11 | 76535064 | 76535188 |
| chr11 | 76588692 | 76588832 |
| chr11 | 76840425 | 76840547 |
| chr11 | 76875757 | 76875881 |
| chr11 | 77050929 | 77051061 |
| chr11 | 77259538 | 77259662 |
| chr11 | 77373419 | 77373541 |
| chr11 | 77522559 | 77522689 |
| chr11 | 77711287 | 77711409 |
| chr11 | 77862629 | 77862801 |
| chr11 | 77959347 | 77959487 |
| chr11 | 78140674 | 78140796 |
| chr11 | 78326745 | 78327000 |
| chr11 | 78362183 | 78362307 |
| chr11 | 78595101 | 78595231 |
| chr11 | 78659899 | 78660033 |
| chr11 | 78823611 | 78823735 |
| chr11 | 79077184 | 79077306 |
| chr11 | 79157986 | 79158108 |
| chr11 | 79366780 | 79366910 |
| chr11 | 79398011 | 79398145 |
| chr11 | 79607707 | 79607831 |
| chr11 | 79768736 | 79768874 |
| chr11 | 79885252 | 79885386 |
| chr11 | 80012466 | 80012594 |
| chr11 | 80195041 | 80195189 |
| chr11 | 80349576 | 80349699 |
| chr11 | 80556324 | 80556450 |
| chr11 | 80596659 | 80596789 |
| chr11 | 80827850 | 80827996 |
| chr11 | 80949288 | 80949428 |
| chr11 | 81052046 | 81052180 |
| chr11 | 81316800 | 81316936 |
| chr11 | 81487228 | 81487366 |
| chr11 | 81553103 | 81553225 |
| chr11 | 81785843 | 81785977 |
| chr11 | 81842728 | 81842878 |
| chr11 | 81986063 | 81986187 |
| chr11 | 82119097 | 82119219 |
| chr11 | 82314351 | 82314489 |
| chr11 | 82535383 | 82535517 |
| chr11 | 82627974 | 82628118 |
| chr11 | 82699106 | 82699244 |
| chr11 | 82955397 | 82955551 |
| chr11 | 83140431 | 83140569 |
| chr11 | 83184400 | 83184530 |
| chr11 | 83440537 | 83440673 |
| chr11 | 83534945 | 83535067 |
| chr11 | 83629338 | 83629474 |
| chr11 | 83879447 | 83879569 |
| chr11 | 83904078 | 83904208 |
| chr11 | 84181976 | 84182154 |
| chr11 | 84273143 | 84273285 |
| chr11 | 84465814 | 84465936 |
| chr11 | 84628937 | 84629061 |
| chr11 | 84685599 | 84685735 |
| chr11 | 84841537 | 84841661 |
| chr11 | 85087374 | 85087498 |
| chr11 | 85114664 | 85114826 |
| chr11 | 85293055 | 85293179 |
| chr11 | 85475943 | 85476075 |
| chr11 | 85691758 | 85691880 |
| chr11 | 85824202 | 85824348 |
| chr11 | 85875596 | 85875720 |
| chr11 | 86012151 | 86012289 |
| chr11 | 86249335 | 86249475 |
| chr11 | 86353882 | 86354004 |
| chr11 | 86491631 | 86491763 |
| chr11 | 86665213 | 86665502 |
| chr11 | 86775067 | 86775191 |
| chr11 | 87001649 | 87001777 |
| chr11 | 87167697 | 87167827 |
| chr11 | 87312691 | 87312839 |
| chr11 | 87456583 | 87456737 |
| chr11 | 87599272 | 87599408 |
| chr11 | 87744943 | 87745205 |
| chr11 | 87881616 | 87881756 |
| chr11 | 87965344 | 87965482 |
| chr11 | 88103119 | 88103265 |
| chr11 | 88297654 | 88297790 |
| chr11 | 88446106 | 88446228 |
| chr11 | 88564271 | 88564392 |
| chr11 | 88593952 | 88594101 |
| chr11 | 88808896 | 88809024 |
| chr11 | 88893672 | 88893794 |
| chr11 | 89000803 | 89000927 |
| chr11 | 89149277 | 89149409 |
| chr11 | 89263319 | 89263465 |
| chr11 | 89391119 | 89391279 |
| chr11 | 89476037 | 89476179 |
| chr11 | 89656371 | 89656492 |
| chr11 | 89830077 | 89830220 |
| chr11 | 89857374 | 89857497 |
| chr11 | 89995965 | 89996091 |
| chr11 | 90095276 | 90095398 |
| chr11 | 90264220 | 90264344 |
| chr11 | 90411860 | 90411986 |
| chr11 | 90622520 | 90622644 |
| chr11 | 90740950 | 90741078 |
| chr11 | 90869920 | 90870066 |
| chr11 | 91079875 | 91079999 |
| chr11 | 91190198 | 91190320 |
| chr11 | 91319540 | 91319664 |
| chr11 | 91424152 | 91424280 |
| chr11 | 91661783 | 91661915 |
| chr11 | 91720348 | 91720492 |
| chr11 | 91895497 | 91895631 |
| chr11 | 92052668 | 92052790 |
| chr11 | 92213807 | 92213935 |
| chr11 | 92353568 | 92353692 |
| chr11 | 92447313 | 92447435 |
| chr11 | 92603241 | 92603373 |
| chr11 | 92742096 | 92742218 |
| chr11 | 92965430 | 92965556 |
| chr11 | 93142597 | 93142723 |
| chr11 | 93265448 | 93265572 |
| chr11 | 93437920 | 93438070 |
| chr11 | 93626573 | 93626713 |
| chr11 | 93704551 | 93704689 |
| chr11 | 93930035 | 93930157 |
| chr11 | 94064290 | 94064414 |
| chr11 | 94167167 | 94167309 |
| chr11 | 94356846 | 94357002 |
| chr11 | 94429466 | 94429610 |
| chr11 | 94557910 | 94558044 |
| chr11 | 94767349 | 94767473 |
| chr11 | 94886580 | 94886720 |
| chr11 | 95078209 | 95078345 |
| chr11 | 95259567 | 95259693 |
| chr11 | 95384974 | 95385108 |
| chr11 | 95508436 | 95508562 |
| chr11 | 95706061 | 95706217 |
| chr11 | 95854026 | 95854178 |
| chr11 | 95938092 | 95938230 |
| chr11 | 96145616 | 96145744 |
| chr11 | 96195635 | 96195763 |
| chr11 | 96465022 | 96465162 |
| chr11 | 96571107 | 96571235 |
| chr11 | 96761734 | 96761866 |
| chr11 | 96832303 | 96832449 |
| chr11 | 96981106 | 96981252 |
| chr11 | 97100809 | 97100933 |
| chr11 | 97321276 | 97321408 |
| chr11 | 97502497 | 97502637 |
| chr11 | 97633101 | 97633231 |
| chr11 | 97826030 | 97826170 |
| chr11 | 97940918 | 97941050 |
| chr11 | 98084985 | 98085141 |
| chr11 | 98209436 | 98209582 |
| chr11 | 98407626 | 98407748 |
| chr11 | 98508395 | 98508519 |
| chr11 | 98657829 | 98657977 |
| chr11 | 98795913 | 98796037 |
| chr11 | 98989102 | 98989226 |
| chr11 | 99112556 | 99112678 |
| chr11 | 99255260 | 99255392 |
| chr11 | 99401157 | 99401283 |
| chr11 | 99496924 | 99497060 |
| chr11 | 99719471 | 99719597 |
| chr11 | 99838130 | 99838260 |
| chr11 | 100080062 | 100080190 |
| chr11 | 100138823 | 100138967 |
| chr11 | 100343971 | 100344109 |
| chr11 | 100447993 | 100448123 |
| chr11 | 100542966 | 100543108 |
| chr11 | 100720801 | 100720927 |
| chr11 | 100975938 | 100976062 |
| chr11 | 100993760 | 100993888 |
| chr11 | 101225339 | 101225465 |
| chr11 | 101410120 | 101410244 |
| chr11 | 101589179 | 101589305 |
| chr11 | 101681430 | 101681564 |
| chr11 | 101861449 | 101861589 |
| chr11 | 101987757 | 101987911 |
| chr11 | 102130423 | 102130545 |
| chr11 | 102290518 | 102290654 |
| chr11 | 102352527 | 102352675 |
| chr11 | 102605497 | 102605621 |
| chr11 | 102720600 | 102720730 |
| chr11 | 102848486 | 102848630 |
| chr11 | 102983530 | 102983656 |
| chr11 | 103173468 | 103173620 |
| chr11 | 103347997 | 103348121 |
| chr11 | 103394119 | 103394241 |
| chr11 | 103682944 | 103683094 |
| chr11 | 103767357 | 103767481 |
| chr11 | 103989112 | 103989238 |
| chr11 | 104026266 | 104026404 |
| chr11 | 104274495 | 104274623 |
| chr11 | 104402681 | 104402807 |
| chr11 | 104500044 | 104500190 |
| chr11 | 104640484 | 104640614 |
| chr11 | 104869141 | 104869281 |
| chr11 | 105025204 | 105025330 |
| chr11 | 105159697 | 105159819 |
| chr11 | 105239405 | 105239529 |
| chr11 | 105441639 | 105441765 |
| chr11 | 105519627 | 105519755 |
| chr11 | 105736654 | 105736788 |
| chr11 | 105868312 | 105868434 |
| chr11 | 105983088 | 105983210 |
| chr11 | 106203786 | 106203930 |
| chr11 | 106311831 | 106311979 |
| chr11 | 106427568 | 106427720 |
| chr11 | 106551508 | 106551636 |
| chr11 | 106756053 | 106756187 |
| chr11 | 106942089 | 106942219 |
| chr11 | 107009136 | 107009276 |
| chr11 | 107203828 | 107203952 |
| chr11 | 107433977 | 107434101 |
| chr11 | 107474715 | 107474847 |
| chr11 | 107720859 | 107720991 |
| chr11 | 107803079 | 107803219 |
| chr11 | 107933027 | 107933159 |
| chr11 | 108095178 | 108095302 |
| chr11 | 108251175 | 108251297 |
| chr11 | 108445490 | 108445614 |
| chr11 | 108589240 | 108589366 |
| chr11 | 108781144 | 108781274 |
| chr11 | 108941197 | 108941337 |
| chr11 | 109085627 | 109085759 |
| chr11 | 109221577 | 109221707 |
| chr11 | 109325241 | 109325387 |
| chr11 | 109450201 | 109450322 |
| chr11 | 109691364 | 109691486 |
| chr11 | 109739878 | 109740016 |
| chr11 | 109906132 | 109906286 |
| chr11 | 110005330 | 110005482 |
| chr11 | 110261334 | 110261456 |
| chr11 | 110307122 | 110307278 |
| chr11 | 110452764 | 110452916 |
| chr11 | 110637091 | 110637237 |
| chr11 | 110797622 | 110797758 |
| chr11 | 110909371 | 110909499 |
| chr11 | 111176482 | 111176608 |
| chr11 | 111217260 | 111217398 |
| chr11 | 111449743 | 111450003 |
| chr11 | 111581433 | 111581555 |
| chr11 | 111648769 | 111648893 |
| chr11 | 111809415 | 111809539 |
| chr11 | 112026619 | 112026741 |
| chr11 | 112217621 | 112217903 |
| chr11 | 112381362 | 112381514 |
| chr11 | 112476957 | 112477083 |
| chr11 | 112680557 | 112680701 |
| chr11 | 112704080 | 112704210 |
| chr11 | 112854225 | 112854357 |
| chr11 | 113077499 | 113077623 |
| chr11 | 113213246 | 113213368 |
| chr11 | 113329720 | 113329848 |
| chr11 | 113555289 | 113555443 |
| chr11 | 113638316 | 113638450 |
| chr11 | 113767164 | 113767306 |
| chr11 | 113972365 | 113972487 |
| chr11 | 114131649 | 114131813 |
| chr11 | 114214149 | 114214291 |
| chr11 | 114387920 | 114388044 |
| chr11 | 114609048 | 114609182 |
| chr11 | 114653795 | 114653919 |
| chr11 | 114805679 | 114805807 |
| chr11 | 114999797 | 114999931 |
| chr11 | 115212345 | 115212483 |
| chr11 | 115341839 | 115341975 |
| chr11 | 115519461 | 115519583 |
| chr11 | 115576548 | 115576672 |
| chr11 | 115770669 | 115770795 |
| chr11 | 115986608 | 115986742 |
| chr11 | 116120469 | 116120595 |
| chr11 | 116203649 | 116203809 |
| chr11 | 116343804 | 116343928 |
| chr11 | 116485509 | 116485685 |
| chr11 | 116601675 | 116601799 |
| chr11 | 116806338 | 116806462 |
| chr11 | 116928432 | 116928568 |
| chr11 | 117120908 | 117121062 |
| chr11 | 117322827 | 117322951 |
| chr11 | 117414319 | 117414455 |
| chr11 | 117563136 | 117563268 |
| chr11 | 117776592 | 117776718 |
| chr11 | 117839223 | 117839359 |
| chr11 | 118042884 | 118043042 |
| chr11 | 118119869 | 118119997 |
| chr11 | 118315035 | 118315163 |
| chr11 | 118449694 | 118449816 |
| chr11 | 118620625 | 118620757 |
| chr11 | 118788043 | 118788167 |
| chr11 | 118846538 | 118846662 |
| chr11 | 119130599 | 119130725 |
| chr11 | 119203453 | 119203593 |
| chr11 | 119306137 | 119306265 |
| chr11 | 119451852 | 119451976 |
| chr11 | 119688366 | 119688500 |
| chr11 | 119767788 | 119767920 |
| chr11 | 119923587 | 119923725 |
| chr11 | 120094340 | 120094488 |
| chr11 | 120303889 | 120304017 |
| chr11 | 120443893 | 120444071 |
| chr11 | 120614474 | 120614622 |
| chr11 | 120733596 | 120733720 |
| chr11 | 120862021 | 120862171 |
| chr11 | 120997404 | 120997544 |
| chr11 | 121179811 | 121179935 |
| chr11 | 121265983 | 121266105 |
| chr11 | 121514935 | 121515057 |
| chr11 | 121690307 | 121690455 |
| chr11 | 121835329 | 121835469 |
| chr11 | 121907973 | 121908107 |
| chr11 | 122099751 | 122099891 |
| chr11 | 122266653 | 122266783 |
| chr11 | 122364657 | 122364781 |
| chr11 | 122456063 | 122456187 |
| chr11 | 122668087 | 122668233 |
| chr11 | 122809131 | 122809257 |
| chr11 | 122962842 | 122962980 |
| chr11 | 123080562 | 123080692 |
| chr11 | 123283840 | 123283986 |
| chr11 | 123408986 | 123409130 |
| chr11 | 123631782 | 123631926 |
| chr11 | 123729408 | 123729554 |
| chr11 | 123917207 | 123917329 |
| chr11 | 124034676 | 124034826 |
| chr11 | 124211835 | 124211957 |
| chr11 | 124245293 | 124245429 |
| chr11 | 124461222 | 124461350 |
| chr11 | 124635056 | 124635178 |
| chr11 | 124839241 | 124839397 |
| chr11 | 124881780 | 124881904 |
| chr11 | 125046838 | 125046966 |
| chr11 | 125162748 | 125162874 |
| chr11 | 125402350 | 125402494 |
| chr11 | 125572826 | 125572952 |
| chr11 | 125657634 | 125657758 |
| chr11 | 125878837 | 125878963 |
| chr11 | 125956731 | 125956855 |
| chr11 | 126124749 | 126124895 |
| chr11 | 126241614 | 126241750 |
| chr11 | 126402048 | 126402170 |
| chr11 | 126630876 | 126631000 |
| chr11 | 126721026 | 126721170 |
| chr11 | 126880263 | 126880389 |
| chr11 | 127005051 | 127005173 |
| chr11 | 127188608 | 127188754 |
| chr11 | 127247438 | 127247578 |
| chr11 | 127520773 | 127520907 |
| chr11 | 127670957 | 127671101 |
| chr11 | 127757480 | 127757620 |
| chr11 | 127941768 | 127941906 |
| chr11 | 128006142 | 128006284 |
| chr11 | 128199652 | 128199784 |
| chr11 | 128355369 | 128355505 |
| chr11 | 128495066 | 128495194 |
| chr11 | 128734372 | 128734498 |
| chr11 | 128808839 | 128808975 |
| chr11 | 128996267 | 128996407 |
| chr11 | 129166961 | 129167087 |
| chr11 | 129208176 | 129208312 |
| chr11 | 129350918 | 129351040 |
| chr11 | 129572788 | 129572926 |
| chr11 | 129657362 | 129657489 |
| chr11 | 129809389 | 129809513 |
| chr11 | 129951832 | 129951960 |
| chr11 | 130240927 | 130241053 |
| chr11 | 130304330 | 130304466 |
| chr11 | 130396142 | 130396266 |
| chr11 | 130660480 | 130660604 |
| chr11 | 130808595 | 130808735 |
| chr11 | 130927562 | 130927684 |
| chr11 | 131018696 | 131018820 |
| chr11 | 131291875 | 131291997 |
| chr11 | 131379403 | 131379531 |
| chr11 | 131500955 | 131501077 |
| chr11 | 131656166 | 131656310 |
| chr11 | 131832638 | 131832764 |
| chr11 | 132010454 | 132010588 |
| chr11 | 132160761 | 132160885 |
| chr11 | 132314400 | 132314528 |
| chr11 | 132434480 | 132434618 |
| chr11 | 132538360 | 132538490 |
| chr11 | 132746554 | 132746678 |
| chr11 | 132861740 | 132861884 |
| chr11 | 132942684 | 132942828 |
| chr11 | 133129676 | 133129818 |
| chr11 | 133267538 | 133267682 |
| chr11 | 133392503 | 133392625 |
| chr11 | 133599869 | 133600015 |
| chr11 | 133757701 | 133757825 |
| chr11 | 133943404 | 133943554 |
| chr11 | 134076868 | 134077028 |
| chr11 | 134256729 | 134256851 |
| chr11 | 134330352 | 134330490 |
| chr11 | 134488095 | 134488251 |
| chr11 | 134739465 | 134739597 |
| chr11 | 134769353 | 134769481 |
| chr11 | 134942878 | 134943014 |
| chr12 | 164247 | 164369 |
| chr12 | 332622 | 332748 |
| chr12 | 570913 | 571035 |
| chr12 | 717417 | 717555 |
| chr12 | 786162 | 786296 |
| chr12 | 975220 | 975348 |
| chr12 | 1163814 | 1163948 |
| chr12 | 1332585 | 1332727 |
| chr12 | 1440792 | 1440916 |
| chr12 | 1572383 | 1572517 |
| chr12 | 1813668 | 1813794 |
| chr12 | 1890969 | 1891093 |
| chr12 | 2111281 | 2111423 |
| chr12 | 2216910 | 2217044 |
| chr12 | 2332768 | 2332920 |
| chr12 | 2501355 | 2501499 |
| chr12 | 2696143 | 2696269 |
| chr12 | 2717106 | 2717238 |
| chr12 | 2897792 | 2897952 |
| chr12 | 3023645 | 3023773 |
| chr12 | 3178434 | 3178556 |
| chr12 | 3376417 | 3376541 |
| chr12 | 3523147 | 3523287 |
| chr12 | 3658268 | 3658420 |
| chr12 | 3807051 | 3807175 |
| chr12 | 3998781 | 3998933 |
| chr12 | 4174621 | 4174763 |
| chr12 | 4299522 | 4299644 |
| chr12 | 4420435 | 4420569 |
| chr12 | 4515653 | 4515777 |
| chr12 | 4688969 | 4689101 |
| chr12 | 4875945 | 4876115 |
| chr12 | 5091297 | 5091437 |
| chr12 | 5254136 | 5254270 |
| chr12 | 5366048 | 5366170 |
| chr12 | 5502625 | 5502769 |
| chr12 | 5660121 | 5660269 |
| chr12 | 5746986 | 5747110 |
| chr12 | 5936211 | 5936345 |
| chr12 | 6102539 | 6102683 |
| chr12 | 6289165 | 6289293 |
| chr12 | 6405500 | 6405640 |
| chr12 | 6605051 | 6605175 |
| chr12 | 6750554 | 6750822 |
| chr12 | 6829300 | 6829503 |
| chr12 | 6993597 | 6993731 |
| chr12 | 7139132 | 7139253 |
| chr12 | 7246004 | 7246127 |
| chr12 | 7400390 | 7400522 |
| chr12 | 7529566 | 7529693 |
| chr12 | 7769736 | 7769862 |
| chr12 | 7849906 | 7850046 |
| chr12 | 8037234 | 8037360 |
| chr12 | 8229901 | 8230025 |
| chr12 | 8308245 | 8308367 |
| chr12 | 8431071 | 8431243 |
| chr12 | 8517737 | 8517857 |
| chr12 | 8671218 | 8671348 |
| chr12 | 8766297 | 8766429 |
| chr12 | 8911421 | 8911553 |
| chr12 | 9013715 | 9013837 |
| chr12 | 9089998 | 9090120 |
| chr12 | 9142257 | 9142379 |
| chr12 | 9183340 | 9183488 |
| chr12 | 9205456 | 9205580 |
| chr12 | 9242888 | 9243010 |
| chr12 | 9291139 | 9291269 |
| chr12 | 9425830 | 9425984 |
| chr12 | 9490366 | 9490514 |
| chr12 | 9746015 | 9746147 |
| chr12 | 9786671 | 9786793 |
| chr12 | 10020918 | 10021058 |
| chr12 | 10114420 | 10114556 |
| chr12 | 10320378 | 10320526 |
| chr12 | 10458135 | 10458257 |
| chr12 | 10546172 | 10546320 |
| chr12 | 10784089 | 10784227 |
| chr12 | 10952993 | 10953129 |
| chr12 | 11045424 | 11045568 |
| chr12 | 11202144 | 11202274 |
| chr12 | 11309956 | 11310088 |
| chr12 | 11502999 | 11503145 |
| chr12 | 11658360 | 11658484 |
| chr12 | 11808460 | 11808594 |
| chr12 | 11916824 | 11916966 |
| chr12 | 12125960 | 12126088 |
| chr12 | 12229872 | 12230002 |
| chr12 | 12422970 | 12423094 |
| chr12 | 12542396 | 12542518 |
| chr12 | 12670185 | 12670315 |
| chr12 | 12798286 | 12798410 |
| chr12 | 13045110 | 13045232 |
| chr12 | 13093270 | 13093422 |
| chr12 | 13351610 | 13351732 |
| chr12 | 13481757 | 13481905 |
| chr12 | 13657632 | 13657756 |
| chr12 | 13765438 | 13765582 |
| chr12 | 13948162 | 13948286 |
| chr12 | 14044424 | 14044546 |
| chr12 | 14239565 | 14239697 |
| chr12 | 14295470 | 14295614 |
| chr12 | 14467887 | 14468011 |
| chr12 | 14571373 | 14571519 |
| chr12 | 14824786 | 14824934 |
| chr12 | 14907987 | 14908131 |
| chr12 | 15140109 | 15140231 |
| chr12 | 15220169 | 15220305 |
| chr12 | 15428853 | 15428999 |
| chr12 | 15491733 | 15491871 |
| chr12 | 15668084 | 15668226 |
| chr12 | 15843576 | 15843710 |
| chr12 | 16033736 | 16033860 |
| chr12 | 16095957 | 16096081 |
| chr12 | 16290865 | 16290987 |
| chr12 | 16395399 | 16395521 |
| chr12 | 16590269 | 16590393 |
| chr12 | 16664240 | 16664416 |
| chr12 | 16948019 | 16948145 |
| chr12 | 17111098 | 17111240 |
| chr12 | 17174112 | 17174240 |
| chr12 | 17355764 | 17355902 |
| chr12 | 17520029 | 17520151 |
| chr12 | 17633555 | 17633679 |
| chr12 | 17837859 | 17837983 |
| chr12 | 17972327 | 17972455 |
| chr12 | 18088492 | 18088614 |
| chr12 | 18252140 | 18252276 |
| chr12 | 18346641 | 18346783 |
| chr12 | 18487772 | 18487912 |
| chr12 | 18618744 | 18618866 |
| chr12 | 18806772 | 18806896 |
| chr12 | 18981743 | 18981885 |
| chr12 | 19075927 | 19076065 |
| chr12 | 19222881 | 19223003 |
| chr12 | 19394104 | 19394230 |
| chr12 | 19600164 | 19600320 |
| chr12 | 19727887 | 19728027 |
| chr12 | 19845243 | 19845369 |
| chr12 | 20064599 | 20064737 |
| chr12 | 20154178 | 20154300 |
| chr12 | 20387103 | 20387239 |
| chr12 | 20543718 | 20543864 |
| chr12 | 20697424 | 20697568 |
| chr12 | 20828460 | 20828586 |
| chr12 | 20984750 | 20984892 |
| chr12 | 21099836 | 21099972 |
| chr12 | 21269052 | 21269174 |
| chr12 | 21321282 | 21321428 |
| chr12 | 21558767 | 21558889 |
| chr12 | 21716933 | 21717069 |
| chr12 | 21803627 | 21803773 |
| chr12 | 21989188 | 21989312 |
| chr12 | 22067131 | 22067265 |
| chr12 | 22265129 | 22265273 |
| chr12 | 22429799 | 22429941 |
| chr12 | 22562444 | 22562567 |
| chr12 | 22751191 | 22751343 |
| chr12 | 22823585 | 22823715 |
| chr12 | 23022775 | 23022919 |
| chr12 | 23211386 | 23211510 |
| chr12 | 23409247 | 23409371 |
| chr12 | 23525284 | 23525418 |
| chr12 | 23600551 | 23600685 |
| chr12 | 23796169 | 23796307 |
| chr12 | 23954305 | 23954435 |
| chr12 | 24075422 | 24075544 |
| chr12 | 24261002 | 24261134 |
| chr12 | 24342047 | 24342183 |
| chr12 | 24488428 | 24488572 |
| chr12 | 24726547 | 24726687 |
| chr12 | 24895515 | 24895655 |
| chr12 | 24960442 | 24960566 |
| chr12 | 25158265 | 25158399 |
| chr12 | 25273703 | 25273849 |
| chr12 | 25401768 | 25401900 |
| chr12 | 25573751 | 25573873 |
| chr12 | 25675624 | 25675752 |
| chr12 | 25955498 | 25955638 |
| chr12 | 25985624 | 25985748 |
| chr12 | 26140314 | 26140458 |
| chr12 | 26306132 | 26306256 |
| chr12 | 26457100 | 26457258 |
| chr12 | 26704059 | 26704207 |
| chr12 | 26765567 | 26765699 |
| chr12 | 26972111 | 26972235 |
| chr12 | 27154456 | 27154590 |
| chr12 | 27214739 | 27214863 |
| chr12 | 27443878 | 27444000 |
| chr12 | 27476459 | 27476593 |
| chr12 | 27727934 | 27728066 |
| chr12 | 27791522 | 27791654 |
| chr12 | 28041988 | 28042128 |
| chr12 | 28117917 | 28118079 |
| chr12 | 28259106 | 28259236 |
| chr12 | 28476757 | 28476891 |
| chr12 | 28636461 | 28636601 |
| chr12 | 28711596 | 28711738 |
| chr12 | 28892795 | 28892921 |
| chr12 | 29087714 | 29087840 |
| chr12 | 29167897 | 29168039 |
| chr12 | 29348069 | 29348193 |
| chr12 | 29519078 | 29519222 |
| chr12 | 29654088 | 29654220 |
| chr12 | 29745804 | 29745942 |
| chr12 | 29905268 | 29905408 |
| chr12 | 30059612 | 30059738 |
| chr12 | 30257019 | 30257159 |
| chr12 | 30316989 | 30317121 |
| chr12 | 30581426 | 30581586 |
| chr12 | 30667268 | 30667392 |
| chr12 | 30803976 | 30804108 |
| chr12 | 30982369 | 30982503 |
| chr12 | 31107881 | 31108005 |
| chr12 | 31235989 | 31236127 |
| chr12 | 31499619 | 31499745 |
| chr12 | 31643015 | 31643141 |
| chr12 | 31741628 | 31741752 |
| chr12 | 31953257 | 31953387 |
| chr12 | 31973847 | 31973983 |
| chr12 | 32223467 | 32223603 |
| chr12 | 32297474 | 32297596 |
| chr12 | 32502007 | 32502139 |
| chr12 | 32649160 | 32649316 |
| chr12 | 32780993 | 32781123 |
| chr12 | 32989165 | 32989291 |
| chr12 | 33122102 | 33122244 |
| chr12 | 33184538 | 33184672 |
| chr12 | 33418103 | 33418249 |
| chr12 | 33603454 | 33603608 |
| chr12 | 33706971 | 33707111 |
| chr12 | 33865137 | 33865283 |
| chr12 | 33979361 | 33979499 |
| chr12 | 34090021 | 34090151 |
| chr12 | 34216773 | 34216944 |
| chr12 | 34370007 | 34370128 |
| chr12 | 34663575 | 34663703 |
| chr12 | 34720324 | 34720464 |
| chr12 | 38282126 | 38282250 |
| chr12 | 38321997 | 38322143 |
| chr12 | 38399735 | 38399856 |
| chr12 | 38495739 | 38495863 |
| chr12 | 38626159 | 38626283 |
| chr12 | 38829807 | 38829943 |
| chr12 | 38919442 | 38919564 |
| chr12 | 39100484 | 39100614 |
| chr12 | 39230433 | 39230581 |
| chr12 | 39356834 | 39356966 |
| chr12 | 39610450 | 39610590 |
| chr12 | 39669667 | 39669793 |
| chr12 | 39832746 | 39832870 |
| chr12 | 39942880 | 39943030 |
| chr12 | 40075429 | 40075557 |
| chr12 | 40287897 | 40288021 |
| chr12 | 40364660 | 40364784 |
| chr12 | 40518915 | 40519041 |
| chr12 | 40732820 | 40732984 |
| chr12 | 40875684 | 40875810 |
| chr12 | 40969750 | 40969884 |
| chr12 | 41242281 | 41242403 |
| chr12 | 41372123 | 41372267 |
| chr12 | 41548497 | 41548623 |
| chr12 | 41653139 | 41653281 |
| chr12 | 41800809 | 41800943 |
| chr12 | 42001812 | 42001944 |
| chr12 | 42141108 | 42141260 |
| chr12 | 42189253 | 42189377 |
| chr12 | 42435023 | 42435151 |
| chr12 | 42470684 | 42470814 |
| chr12 | 42704880 | 42705034 |
| chr12 | 42816210 | 42816358 |
| chr12 | 42981958 | 42982082 |
| chr12 | 43121307 | 43121465 |
| chr12 | 43301993 | 43302127 |
| chr12 | 43429439 | 43429563 |
| chr12 | 43521152 | 43521288 |
| chr12 | 43719868 | 43719994 |
| chr12 | 43959402 | 43959548 |
| chr12 | 44105228 | 44105378 |
| chr12 | 44202827 | 44202959 |
| chr12 | 44292663 | 44292797 |
| chr12 | 44555364 | 44555496 |
| chr12 | 44568758 | 44568886 |
| chr12 | 44750943 | 44751097 |
| chr12 | 44942998 | 44943138 |
| chr12 | 45142710 | 45142836 |
| chr12 | 45234937 | 45235061 |
| chr12 | 45344482 | 45344612 |
| chr12 | 45487614 | 45487738 |
| chr12 | 45764201 | 45764329 |
| chr12 | 45871552 | 45871682 |
| chr12 | 45931147 | 45931289 |
| chr12 | 46144777 | 46144905 |
| chr12 | 46329102 | 46329242 |
| chr12 | 46475992 | 46476116 |
| chr12 | 46658019 | 46658143 |
| chr12 | 46720765 | 46720893 |
| chr12 | 46899723 | 46899875 |
| chr12 | 47102888 | 47103014 |
| chr12 | 47220329 | 47220453 |
| chr12 | 47393962 | 47394096 |
| chr12 | 47496248 | 47496372 |
| chr12 | 47573341 | 47573465 |
| chr12 | 47723068 | 47723204 |
| chr12 | 47996498 | 47996630 |
| chr12 | 48097230 | 48097402 |
| chr12 | 48303517 | 48303649 |
| chr12 | 48366800 | 48366924 |
| chr12 | 48503827 | 48503957 |
| chr12 | 48690311 | 48690451 |
| chr12 | 48768493 | 48768643 |
| chr12 | 48959990 | 48960144 |
| chr12 | 49140781 | 49140909 |
| chr12 | 49301149 | 49301279 |
| chr12 | 49412806 | 49412928 |
| chr12 | 49661627 | 49661753 |
| chr12 | 49787430 | 49787623 |
| chr12 | 49833941 | 49834063 |
| chr12 | 50010745 | 50010867 |
| chr12 | 50177236 | 50177374 |
| chr12 | 50412851 | 50412983 |
| chr12 | 50460741 | 50460863 |
| chr12 | 50671316 | 50671458 |
| chr12 | 50806549 | 50806689 |
| chr12 | 50882712 | 50882935 |
| chr12 | 51059601 | 51059741 |
| chr12 | 51204848 | 51204978 |
| chr12 | 51316208 | 51316364 |
| chr12 | 51537106 | 51537264 |
| chr12 | 51650876 | 51650998 |
| chr12 | 51777585 | 51777711 |
| chr12 | 51952824 | 51952968 |
| chr12 | 52076634 | 52076758 |
| chr12 | 52260190 | 52260318 |
| chr12 | 52466387 | 52466523 |
| chr12 | 52521992 | 52522126 |
| chr12 | 52691013 | 52691151 |
| chr12 | 52838461 | 52838607 |
| chr12 | 53050296 | 53050422 |
| chr12 | 53164219 | 53164347 |
| chr12 | 53318509 | 53318635 |
| chr12 | 53552573 | 53552719 |
| chr12 | 53634578 | 53634710 |
| chr12 | 53834903 | 53835031 |
| chr12 | 53995433 | 53995579 |
| chr12 | 54100846 | 54100968 |
| chr12 | 54277694 | 54277826 |
| chr12 | 54376212 | 54376334 |
| chr12 | 54544889 | 54545013 |
| chr12 | 54674181 | 54674307 |
| chr12 | 54908627 | 54908757 |
| chr12 | 55057360 | 55057486 |
| chr12 | 55138417 | 55138541 |
| chr12 | 55241536 | 55241692 |
| chr12 | 55371637 | 55371759 |
| chr12 | 55623486 | 55623610 |
| chr12 | 55776880 | 55777012 |
| chr12 | 55869656 | 55869780 |
| chr12 | 56038945 | 56039073 |
| chr12 | 56244215 | 56244488 |
| chr12 | 56276141 | 56276413 |
| chr12 | 56330496 | 56330620 |
| chr12 | 56395322 | 56395460 |
| chr12 | 56482138 | 56482268 |
| chr12 | 56495899 | 56496174 |
| chr12 | 56552521 | 56552850 |
| chr12 | 56577666 | 56577942 |
| chr12 | 56631907 | 56632075 |
| chr12 | 56833699 | 56833829 |
| chr12 | 56983170 | 56983308 |
| chr12 | 57158119 | 57158255 |
| chr12 | 57278328 | 57278456 |
| chr12 | 57324420 | 57324546 |
| chr12 | 57489574 | 57489706 |
| chr12 | 57705579 | 57705705 |
| chr12 | 57900251 | 57900389 |
| chr12 | 57930296 | 57930432 |
| chr12 | 58099662 | 58099784 |
| chr12 | 58259478 | 58259612 |
| chr12 | 58498144 | 58498266 |
| chr12 | 58527261 | 58527387 |
| chr12 | 58750651 | 58750795 |
| chr12 | 58945221 | 58945347 |
| chr12 | 59017809 | 59017959 |
| chr12 | 59122739 | 59122861 |
| chr12 | 59403058 | 59403202 |
| chr12 | 59430684 | 59430830 |
| chr12 | 59634323 | 59634447 |
| chr12 | 59809204 | 59809330 |
| chr12 | 59888120 | 59888244 |
| chr12 | 60065138 | 60065262 |
| chr12 | 60275577 | 60275719 |
| chr12 | 60359887 | 60360011 |
| chr12 | 60542732 | 60542854 |
| chr12 | 60742921 | 60743043 |
| chr12 | 60846356 | 60846480 |
| chr12 | 61023669 | 61023797 |
| chr12 | 61132191 | 61132319 |
| chr12 | 61331162 | 61331286 |
| chr12 | 61439729 | 61439871 |
| chr12 | 61613652 | 61613778 |
| chr12 | 61727437 | 61727593 |
| chr12 | 61883357 | 61883497 |
| chr12 | 62024109 | 62024239 |
| chr12 | 62153028 | 62153150 |
| chr12 | 62371781 | 62371905 |
| chr12 | 62489302 | 62489428 |
| chr12 | 62592789 | 62592925 |
| chr12 | 62768014 | 62768136 |
| chr12 | 62934358 | 62934492 |
| chr12 | 63069330 | 63069456 |
| chr12 | 63240582 | 63240720 |
| chr12 | 63450916 | 63451052 |
| chr12 | 63576616 | 63576770 |
| chr12 | 63745408 | 63745554 |
| chr12 | 63836523 | 63836647 |
| chr12 | 63934025 | 63934149 |
| chr12 | 64016235 | 64016365 |
| chr12 | 64076863 | 64076985 |
| chr12 | 64324194 | 64324320 |
| chr12 | 64484660 | 64484794 |
| chr12 | 64553945 | 64554083 |
| chr12 | 64805253 | 64805375 |
| chr12 | 64896642 | 64896784 |
| chr12 | 65089225 | 65089361 |
| chr12 | 65195889 | 65196029 |
| chr12 | 65285869 | 65285993 |
| chr12 | 65553710 | 65553831 |
| chr12 | 65581061 | 65581187 |
| chr12 | 65838875 | 65839017 |
| chr12 | 65967097 | 65967235 |
| chr12 | 66084176 | 66084300 |
| chr12 | 66261891 | 66262017 |
| chr12 | 66393773 | 66393903 |
| chr12 | 66572339 | 66572465 |
| chr12 | 66750339 | 66750524 |
| chr12 | 66848365 | 66848507 |
| chr12 | 66980534 | 66980662 |
| chr12 | 67067057 | 67067181 |
| chr12 | 67262728 | 67262858 |
| chr12 | 67380948 | 67381086 |
| chr12 | 67658473 | 67658597 |
| chr12 | 67692094 | 67692236 |
| chr12 | 67825493 | 67825629 |
| chr12 | 68097544 | 68097682 |
| chr12 | 68172486 | 68172614 |
| chr12 | 68281705 | 68281829 |
| chr12 | 68562269 | 68562393 |
| chr12 | 68702936 | 68703068 |
| chr12 | 68742889 | 68743011 |
| chr12 | 68999319 | 68999453 |
| chr12 | 69084038 | 69084188 |
| chr12 | 69214633 | 69214763 |
| chr12 | 69396659 | 69396799 |
| chr12 | 69479027 | 69479185 |
| chr12 | 69671986 | 69672112 |
| chr12 | 69786457 | 69786581 |
| chr12 | 69958229 | 69958353 |
| chr12 | 70065536 | 70065660 |
| chr12 | 70361797 | 70361921 |
| chr12 | 70501641 | 70501769 |
| chr12 | 70653665 | 70653787 |
| chr12 | 70803699 | 70803821 |
| chr12 | 70832504 | 70832656 |
| chr12 | 71026796 | 71026920 |
| chr12 | 71237237 | 71237359 |
| chr12 | 71329461 | 71329589 |
| chr12 | 71529401 | 71529549 |
| chr12 | 71618755 | 71618879 |
| chr12 | 71761209 | 71761347 |
| chr12 | 71869835 | 71869973 |
| chr12 | 72052949 | 72053073 |
| chr12 | 72310873 | 72311011 |
| chr12 | 72391009 | 72391149 |
| chr12 | 72504798 | 72504944 |
| chr12 | 72683900 | 72684038 |
| chr12 | 72904220 | 72904376 |
| chr12 | 73000111 | 73000259 |
| chr12 | 73177855 | 73177977 |
| chr12 | 73299462 | 73299592 |
| chr12 | 73376771 | 73376895 |
| chr12 | 73603520 | 73603642 |
| chr12 | 73750088 | 73750212 |
| chr12 | 73929937 | 73930067 |
| chr12 | 74063865 | 74063987 |
| chr12 | 74227638 | 74227794 |
| chr12 | 74349809 | 74349931 |
| chr12 | 74509540 | 74509670 |
| chr12 | 74680727 | 74680849 |
| chr12 | 74798128 | 74798252 |
| chr12 | 74866663 | 74866785 |
| chr12 | 75062530 | 75062662 |
| chr12 | 75213871 | 75213995 |
| chr12 | 75463541 | 75463683 |
| chr12 | 75595320 | 75595444 |
| chr12 | 75628761 | 75628889 |
| chr12 | 75903101 | 75903259 |
| chr12 | 76057731 | 76057861 |
| chr12 | 76158991 | 76159149 |
| chr12 | 76287334 | 76287478 |
| chr12 | 76490399 | 76490523 |
| chr12 | 76580599 | 76580723 |
| chr12 | 76672763 | 76672909 |
| chr12 | 76843853 | 76843999 |
| chr12 | 77098859 | 77098987 |
| chr12 | 77201112 | 77201238 |
| chr12 | 77252524 | 77252652 |
| chr12 | 77326240 | 77326364 |
| chr12 | 77460049 | 77460175 |
| chr12 | 77579747 | 77579887 |
| chr12 | 77773461 | 77773601 |
| chr12 | 77878783 | 77878907 |
| chr12 | 78148499 | 78148623 |
| chr12 | 78232768 | 78232908 |
| chr12 | 78416776 | 78416898 |
| chr12 | 78548243 | 78548375 |
| chr12 | 78729748 | 78729882 |
| chr12 | 78877725 | 78877869 |
| chr12 | 79039489 | 79039615 |
| chr12 | 79113375 | 79113510 |
| chr12 | 79266187 | 79266341 |
| chr12 | 79450350 | 79450472 |
| chr12 | 79591048 | 79591182 |
| chr12 | 79779363 | 79779491 |
| chr12 | 79829805 | 79829937 |
| chr12 | 80093241 | 80093365 |
| chr12 | 80195433 | 80195557 |
| chr12 | 80394725 | 80394855 |
| chr12 | 80562827 | 80562973 |
| chr12 | 80691892 | 80692149 |
| chr12 | 80743276 | 80743414 |
| chr12 | 80883818 | 80883962 |
| chr12 | 81160168 | 81160306 |
| chr12 | 81255883 | 81256025 |
| chr12 | 81340602 | 81340726 |
| chr12 | 81552626 | 81552760 |
| chr12 | 81728078 | 81728208 |
| chr12 | 81792671 | 81792793 |
| chr12 | 81998418 | 81998554 |
| chr12 | 82172318 | 82172442 |
| chr12 | 82271537 | 82271661 |
| chr12 | 82492601 | 82492727 |
| chr12 | 82563949 | 82564077 |
| chr12 | 82673583 | 82673706 |
| chr12 | 82834388 | 82834584 |
| chr12 | 83065171 | 83065293 |
| chr12 | 83146578 | 83146748 |
| chr12 | 83325446 | 83325578 |
| chr12 | 83477099 | 83477221 |
| chr12 | 83678817 | 83678943 |
| chr12 | 83773977 | 83774115 |
| chr12 | 83962826 | 83962948 |
| chr12 | 84133734 | 84133858 |
| chr12 | 84241838 | 84241962 |
| chr12 | 84384876 | 84385004 |
| chr12 | 84589076 | 84589252 |
| chr12 | 84743938 | 84744090 |
| chr12 | 84772171 | 84772309 |
| chr12 | 85012253 | 85012401 |
| chr12 | 85076147 | 85076275 |
| chr12 | 85316936 | 85317066 |
| chr12 | 85487356 | 85487496 |
| chr12 | 85602503 | 85602659 |
| chr12 | 85767600 | 85767742 |
| chr12 | 85847115 | 85847241 |
| chr12 | 85964943 | 85965091 |
| chr12 | 86120748 | 86120898 |
| chr12 | 86285718 | 86285848 |
| chr12 | 86474502 | 86474624 |
| chr12 | 86704734 | 86704884 |
| chr12 | 86852031 | 86852161 |
| chr12 | 86936041 | 86936167 |
| chr12 | 87048287 | 87048419 |
| chr12 | 87177928 | 87178052 |
| chr12 | 87435258 | 87435384 |
| chr12 | 87545995 | 87546119 |
| chr12 | 87723283 | 87723417 |
| chr12 | 87794662 | 87794784 |
| chr12 | 88005538 | 88005708 |
| chr12 | 88186359 | 88186501 |
| chr12 | 88268390 | 88268514 |
| chr12 | 88444499 | 88444678 |
| chr12 | 88528699 | 88528820 |
| chr12 | 88722706 | 88722882 |
| chr12 | 88960661 | 88960785 |
| chr12 | 89013060 | 89013194 |
| chr12 | 89209499 | 89209623 |
| chr12 | 89290455 | 89290609 |
| chr12 | 89528902 | 89529056 |
| chr12 | 89593882 | 89594008 |
| chr12 | 89757018 | 89757281 |
| chr12 | 89871128 | 89871266 |
| chr12 | 90062324 | 90062448 |
| chr12 | 90199481 | 90199605 |
| chr12 | 90402617 | 90402749 |
| chr12 | 90559415 | 90559563 |
| chr12 | 90702840 | 90702974 |
| chr12 | 90883147 | 90883301 |
| chr12 | 90975530 | 90975658 |
| chr12 | 91122056 | 91122194 |
| chr12 | 91264551 | 91264693 |
| chr12 | 91488077 | 91488227 |
| chr12 | 91598184 | 91598305 |
| chr12 | 91669665 | 91669789 |
| chr12 | 91918465 | 91918589 |
| chr12 | 92043363 | 92043487 |
| chr12 | 92219928 | 92220054 |
| chr12 | 92384572 | 92384694 |
| chr12 | 92518615 | 92518765 |
| chr12 | 92586013 | 92586135 |
| chr12 | 92724116 | 92724244 |
| chr12 | 92932829 | 92932961 |
| chr12 | 93102249 | 93102383 |
| chr12 | 93273473 | 93273594 |
| chr12 | 93398625 | 93398751 |
| chr12 | 93575355 | 93575511 |
| chr12 | 93709802 | 93709928 |
| chr12 | 93899469 | 93899603 |
| chr12 | 93973445 | 93973571 |
| chr12 | 94077192 | 94077314 |
| chr12 | 94324247 | 94324371 |
| chr12 | 94372803 | 94372927 |
| chr12 | 94529140 | 94529276 |
| chr12 | 94709639 | 94709775 |
| chr12 | 94858953 | 94859085 |
| chr12 | 95107387 | 95107525 |
| chr12 | 95208579 | 95208703 |
| chr12 | 95404606 | 95404728 |
| chr12 | 95496533 | 95496659 |
| chr12 | 95601540 | 95601670 |
| chr12 | 95719542 | 95719670 |
| chr12 | 96002389 | 96002519 |
| chr12 | 96036267 | 96036391 |
| chr12 | 96177406 | 96177546 |
| chr12 | 96344182 | 96344328 |
| chr12 | 96518810 | 96518932 |
| chr12 | 96647401 | 96647555 |
| chr12 | 96785977 | 96786113 |
| chr12 | 97063756 | 97063882 |
| chr12 | 97146519 | 97146643 |
| chr12 | 97266427 | 97266551 |
| chr12 | 97429356 | 97429494 |
| chr12 | 97562629 | 97562775 |
| chr12 | 97779291 | 97779417 |
| chr12 | 97885075 | 97885213 |
| chr12 | 98062930 | 98063062 |
| chr12 | 98115097 | 98115229 |
| chr12 | 98354485 | 98354615 |
| chr12 | 98538566 | 98538688 |
| chr12 | 98628908 | 98629036 |
| chr12 | 98748302 | 98748573 |
| chr12 | 98983160 | 98983302 |
| chr12 | 99067500 | 99067632 |
| chr12 | 99257603 | 99257727 |
| chr12 | 99393326 | 99393456 |
| chr12 | 99532387 | 99532527 |
| chr12 | 99698401 | 99698533 |
| chr12 | 99896274 | 99896420 |
| chr12 | 99987717 | 99987863 |
| chr12 | 100071766 | 100071894 |
| chr12 | 100255336 | 100255458 |
| chr12 | 100401831 | 100402105 |
| chr12 | 100643359 | 100643575 |
| chr12 | 100780946 | 100781070 |
| chr12 | 100950333 | 100950459 |
| chr12 | 101023491 | 101023627 |
| chr12 | 101215725 | 101215859 |
| chr12 | 101392643 | 101392779 |
| chr12 | 101468835 | 101468959 |
| chr12 | 101663585 | 101663707 |
| chr12 | 101733846 | 101733970 |
| chr12 | 101941031 | 101941153 |
| chr12 | 102092296 | 102092420 |
| chr12 | 102313431 | 102313559 |
| chr12 | 102352667 | 102352789 |
| chr12 | 102557123 | 102557247 |
| chr12 | 102654496 | 102654626 |
| chr12 | 102912520 | 102912646 |
| chr12 | 103012236 | 103012366 |
| chr12 | 103140291 | 103140415 |
| chr12 | 103256461 | 103256605 |
| chr12 | 103387658 | 103387788 |
| chr12 | 103568272 | 103568396 |
| chr12 | 103772907 | 103773045 |
| chr12 | 103876342 | 103876466 |
| chr12 | 104034865 | 104035013 |
| chr12 | 104261571 | 104261709 |
| chr12 | 104325051 | 104325197 |
| chr12 | 104448421 | 104448543 |
| chr12 | 104620138 | 104620290 |
| chr12 | 104813519 | 104813643 |
| chr12 | 104889545 | 104889667 |
| chr12 | 105032884 | 105033022 |
| chr12 | 105236595 | 105236731 |
| chr12 | 105410700 | 105410832 |
| chr12 | 105581203 | 105581329 |
| chr12 | 105743795 | 105743927 |
| chr12 | 105898554 | 105898678 |
| chr12 | 105955419 | 105955563 |
| chr12 | 106210067 | 106210191 |
| chr12 | 106258663 | 106258787 |
| chr12 | 106477348 | 106477470 |
| chr12 | 106649150 | 106649276 |
| chr12 | 106739665 | 106739797 |
| chr12 | 106917128 | 106917266 |
| chr12 | 106980200 | 106980338 |
| chr12 | 107205357 | 107205485 |
| chr12 | 107370132 | 107370258 |
| chr12 | 107488118 | 107488260 |
| chr12 | 107615730 | 107615882 |
| chr12 | 107861766 | 107861910 |
| chr12 | 107993312 | 107993466 |
| chr12 | 108152579 | 108152707 |
| chr12 | 108275507 | 108275631 |
| chr12 | 108404508 | 108404646 |
| chr12 | 108465915 | 108466065 |
| chr12 | 108710989 | 108711113 |
| chr12 | 108845352 | 108845474 |
| chr12 | 109037222 | 109037358 |
| chr12 | 109116683 | 109116938 |
| chr12 | 109304554 | 109304698 |
| chr12 | 109457171 | 109457295 |
| chr12 | 109646650 | 109646810 |
| chr12 | 109808487 | 109808609 |
| chr12 | 109908967 | 109909093 |
| chr12 | 110016374 | 110016500 |
| chr12 | 110178409 | 110178563 |
| chr12 | 110345009 | 110345159 |
| chr12 | 110497456 | 110497592 |
| chr12 | 110609626 | 110609766 |
| chr12 | 110738644 | 110738766 |
| chr12 | 110984688 | 110984899 |
| chr12 | 111075771 | 111075895 |
| chr12 | 111205683 | 111205934 |
| chr12 | 111455110 | 111455360 |
| chr12 | 111587894 | 111588016 |
| chr12 | 111660525 | 111660647 |
| chr12 | 111818445 | 111818575 |
| chr12 | 112007988 | 112008129 |
| chr12 | 112113255 | 112113465 |
| chr12 | 112248097 | 112248225 |
| chr12 | 112486760 | 112486892 |
| chr12 | 112571079 | 112571203 |
| chr12 | 112760388 | 112760522 |
| chr12 | 112849060 | 112849226 |
| chr12 | 112983078 | 112983202 |
| chr12 | 113209431 | 113209591 |
| chr12 | 113311634 | 113311758 |
| chr12 | 113509963 | 113510085 |
| chr12 | 113566342 | 113566609 |
| chr12 | 113810768 | 113810982 |
| chr12 | 113894866 | 113895115 |
| chr12 | 114117766 | 114117890 |
| chr12 | 114219781 | 114219909 |
| chr12 | 114326495 | 114326633 |
| chr12 | 114491069 | 114491227 |
| chr12 | 114661026 | 114661152 |
| chr12 | 114802325 | 114802449 |
| chr12 | 114946517 | 114946645 |
| chr12 | 115208115 | 115208251 |
| chr12 | 115334920 | 115335054 |
| chr12 | 115500655 | 115500779 |
| chr12 | 115619188 | 115619322 |
| chr12 | 115692613 | 115692745 |
| chr12 | 115827005 | 115827135 |
| chr12 | 116077867 | 116077989 |
| chr12 | 116170107 | 116170231 |
| chr12 | 116314793 | 116314933 |
| chr12 | 116536238 | 116536480 |
| chr12 | 116621103 | 116621233 |
| chr12 | 116739773 | 116739921 |
| chr12 | 116962666 | 116962814 |
| chr12 | 117073292 | 117073436 |
| chr12 | 117291057 | 117291193 |
| chr12 | 117370028 | 117370152 |
| chr12 | 117591991 | 117592115 |
| chr12 | 117649100 | 117649230 |
| chr12 | 117835188 | 117835326 |
| chr12 | 118026866 | 118027000 |
| chr12 | 118191006 | 118191138 |
| chr12 | 118327451 | 118327575 |
| chr12 | 118478042 | 118478166 |
| chr12 | 118650292 | 118650422 |
| chr12 | 118808678 | 118808802 |
| chr12 | 118866936 | 118867112 |
| chr12 | 119054925 | 119055055 |
| chr12 | 119189036 | 119189168 |
| chr12 | 119399095 | 119399229 |
| chr12 | 119499521 | 119499643 |
| chr12 | 119622511 | 119622641 |
| chr12 | 119819142 | 119819284 |
| chr12 | 119873868 | 119874002 |
| chr12 | 120074462 | 120074600 |
| chr12 | 120228702 | 120228826 |
| chr12 | 120415028 | 120415152 |
| chr12 | 120511492 | 120511618 |
| chr12 | 120615912 | 120616042 |
| chr12 | 120900238 | 120900360 |
| chr12 | 121036666 | 121036810 |
| chr12 | 121082389 | 121082517 |
| chr12 | 121280803 | 121280925 |
| chr12 | 121376326 | 121376450 |
| chr12 | 121622913 | 121623057 |
| chr12 | 121732128 | 121732372 |
| chr12 | 121931163 | 121931287 |
| chr12 | 121983413 | 121983551 |
| chr12 | 122177244 | 122177372 |
| chr12 | 122367958 | 122368084 |
| chr12 | 122527735 | 122527862 |
| chr12 | 122598982 | 122599128 |
| chr12 | 122823687 | 122823843 |
| chr12 | 122887443 | 122887587 |
| chr12 | 123131571 | 123131709 |
| chr12 | 123272335 | 123272609 |
| chr12 | 123368357 | 123368507 |
| chr12 | 123528315 | 123528441 |
| chr12 | 123700950 | 123701082 |
| chr12 | 123828946 | 123829090 |
| chr12 | 124057123 | 124057255 |
| chr12 | 124130318 | 124130476 |
| chr12 | 124346529 | 124346651 |
| chr12 | 124460621 | 124460775 |
| chr12 | 124602592 | 124602734 |
| chr12 | 124772100 | 124772240 |
| chr12 | 124896794 | 124896920 |
| chr12 | 125077018 | 125077152 |
| chr12 | 125151004 | 125151158 |
| chr12 | 125391017 | 125391145 |
| chr12 | 125494862 | 125494996 |
| chr12 | 125565894 | 125566022 |
| chr12 | 125840300 | 125840422 |
| chr12 | 125991247 | 125991385 |
| chr12 | 126089765 | 126089897 |
| chr12 | 126231634 | 126231762 |
| chr12 | 126344097 | 126344221 |
| chr12 | 126578878 | 126579008 |
| chr12 | 126691023 | 126691153 |
| chr12 | 126862607 | 126862743 |
| chr12 | 126954105 | 126954355 |
| chr12 | 127069001 | 127069131 |
| chr12 | 127238471 | 127238607 |
| chr12 | 127394328 | 127394452 |
| chr12 | 127585302 | 127585448 |
| chr12 | 127664817 | 127664957 |
| chr12 | 127899277 | 127899403 |
| chr12 | 128015983 | 128016121 |
| chr12 | 128238297 | 128238419 |
| chr12 | 128347559 | 128347699 |
| chr12 | 128501846 | 128501968 |
| chr12 | 128625063 | 128625191 |
| chr12 | 128788134 | 128788256 |
| chr12 | 128923636 | 128923780 |
| chr12 | 129092061 | 129092195 |
| chr12 | 129184105 | 129184247 |
| chr12 | 129400274 | 129400398 |
| chr12 | 129494308 | 129494432 |
| chr12 | 129645163 | 129645305 |
| chr12 | 129874694 | 129874836 |
| chr12 | 129918830 | 129918956 |
| chr12 | 130156927 | 130157051 |
| chr12 | 130256238 | 130256372 |
| chr12 | 130443271 | 130443393 |
| chr12 | 130660843 | 130660967 |
| chr12 | 130721577 | 130721703 |
| chr12 | 130850304 | 130850426 |
| chr12 | 130972995 | 130973149 |
| chr12 | 131223890 | 131224028 |
| chr12 | 131410497 | 131410629 |
| chr12 | 131554445 | 131554567 |
| chr12 | 131645776 | 131645906 |
| chr12 | 131730227 | 131730349 |
| chr12 | 131934735 | 131934865 |
| chr12 | 132068939 | 132069085 |
| chr12 | 132242175 | 132242301 |
| chr12 | 132446655 | 132446781 |
| chr12 | 132609625 | 132609767 |
| chr12 | 132706500 | 132706621 |
| chr12 | 132835620 | 132835748 |
| chr12 | 132965828 | 132965952 |
| chr12 | 133192499 | 133192621 |
| chr12 | 133289920 | 133290080 |
| chr12 | 133401040 | 133401164 |
| chr12 | 133610010 | 133610280 |
| chr12 | 133713862 | 133714056 |
| chr12 | 133830141 | 133830273 |
| chr13 | 19057984 | 19058104 |
| chr13 | 19064249 | 19064371 |
| chr13 | 19451840 | 19451974 |
| chr13 | 19748657 | 19748789 |
| chr13 | 19886946 | 19887098 |
| chr13 | 19938568 | 19938696 |
| chr13 | 20146359 | 20146493 |
| chr13 | 20213228 | 20213349 |
| chr13 | 20494474 | 20494606 |
| chr13 | 20544878 | 20545298 |
| chr13 | 20756754 | 20757092 |
| chr13 | 20822064 | 20822190 |
| chr13 | 21085713 | 21085837 |
| chr13 | 21247077 | 21247213 |
| chr13 | 21343155 | 21343279 |
| chr13 | 21428332 | 21428454 |
| chr13 | 21572080 | 21572206 |
| chr13 | 21759540 | 21759668 |
| chr13 | 21863572 | 21863693 |
| chr13 | 22091069 | 22091221 |
| chr13 | 22217524 | 22217662 |
| chr13 | 22306429 | 22306573 |
| chr13 | 22460688 | 22460828 |
| chr13 | 22740973 | 22741097 |
| chr13 | 22854151 | 22854275 |
| chr13 | 23016430 | 23016564 |
| chr13 | 23120800 | 23120934 |
| chr13 | 23293075 | 23293201 |
| chr13 | 23371709 | 23371835 |
| chr13 | 23625594 | 23625734 |
| chr13 | 23662634 | 23662790 |
| chr13 | 23846771 | 23846939 |
| chr13 | 24067740 | 24067880 |
| chr13 | 24230771 | 24230899 |
| chr13 | 24280997 | 24281121 |
| chr13 | 24426475 | 24426599 |
| chr13 | 24698720 | 24698860 |
| chr13 | 24848305 | 24848443 |
| chr13 | 24951434 | 24951558 |
| chr13 | 25041251 | 25041379 |
| chr13 | 25237414 | 25237544 |
| chr13 | 25406380 | 25406506 |
| chr13 | 25584807 | 25584931 |
| chr13 | 25716410 | 25716552 |
| chr13 | 25823123 | 25823267 |
| chr13 | 25986465 | 25986597 |
| chr13 | 26092486 | 26092664 |
| chr13 | 26235741 | 26235865 |
| chr13 | 26501817 | 26501955 |
| chr13 | 26551927 | 26552069 |
| chr13 | 26732967 | 26733101 |
| chr13 | 26900914 | 26901056 |
| chr13 | 27089982 | 27090108 |
| chr13 | 27190182 | 27190306 |
| chr13 | 27303283 | 27303405 |
| chr13 | 27533558 | 27533708 |
| chr13 | 27588041 | 27588173 |
| chr13 | 27831372 | 27831524 |
| chr13 | 27879427 | 27879565 |
| chr13 | 28116505 | 28116647 |
| chr13 | 28217420 | 28217546 |
| chr13 | 28427202 | 28427350 |
| chr13 | 28512661 | 28512791 |
| chr13 | 28714992 | 28715128 |
| chr13 | 28892895 | 28893019 |
| chr13 | 28982060 | 28982198 |
| chr13 | 29143670 | 29143794 |
| chr13 | 29210679 | 29210827 |
| chr13 | 29469212 | 29469334 |
| chr13 | 29625836 | 29625988 |
| chr13 | 29739584 | 29739718 |
| chr13 | 29873441 | 29873605 |
| chr13 | 29987147 | 29987299 |
| chr13 | 30222246 | 30222372 |
| chr13 | 30364167 | 30364307 |
| chr13 | 30426317 | 30426441 |
| chr13 | 30642617 | 30642741 |
| chr13 | 30737703 | 30737837 |
| chr13 | 30865283 | 30865423 |
| chr13 | 31102723 | 31102845 |
| chr13 | 31298818 | 31298944 |
| chr13 | 31310495 | 31310627 |
| chr13 | 31493167 | 31493309 |
| chr13 | 31669573 | 31669703 |
| chr13 | 31874362 | 31874530 |
| chr13 | 31910127 | 31910249 |
| chr13 | 32118289 | 32118415 |
| chr13 | 32331745 | 32331869 |
| chr13 | 32353717 | 32353853 |
| chr13 | 32505443 | 32505583 |
| chr13 | 32800639 | 32800785 |
| chr13 | 32938835 | 32938957 |
| chr13 | 32970530 | 32970660 |
| chr13 | 33244885 | 33245015 |
| chr13 | 33333308 | 33333452 |
| chr13 | 33485206 | 33485462 |
| chr13 | 33670082 | 33670220 |
| chr13 | 33707911 | 33708045 |
| chr13 | 33991843 | 33991967 |
| chr13 | 34128816 | 34128940 |
| chr13 | 34225504 | 34225628 |
| chr13 | 34398582 | 34398706 |
| chr13 | 34502801 | 34502939 |
| chr13 | 34744837 | 34744963 |
| chr13 | 34791570 | 34791698 |
| chr13 | 34959710 | 34959842 |
| chr13 | 35193948 | 35194070 |
| chr13 | 35297240 | 35297390 |
| chr13 | 35390529 | 35390653 |
| chr13 | 35525859 | 35525983 |
| chr13 | 35731918 | 35732052 |
| chr13 | 35877377 | 35877511 |
| chr13 | 36014937 | 36015067 |
| chr13 | 36162364 | 36162490 |
| chr13 | 36359050 | 36359176 |
| chr13 | 36419860 | 36419984 |
| chr13 | 36628851 | 36628985 |
| chr13 | 36772144 | 36772266 |
| chr13 | 36959282 | 36959404 |
| chr13 | 37038684 | 37038806 |
| chr13 | 37237374 | 37237496 |
| chr13 | 37400547 | 37400669 |
| chr13 | 37551185 | 37551309 |
| chr13 | 37662274 | 37662398 |
| chr13 | 37768412 | 37768560 |
| chr13 | 38032695 | 38032817 |
| chr13 | 38197548 | 38197674 |
| chr13 | 38280340 | 38280464 |
| chr13 | 38367928 | 38368066 |
| chr13 | 38512448 | 38512582 |
| chr13 | 38769241 | 38769365 |
| chr13 | 38857581 | 38857737 |
| chr13 | 38989242 | 38989368 |
| chr13 | 39138032 | 39138162 |
| chr13 | 39275389 | 39275531 |
| chr13 | 39457768 | 39457900 |
| chr13 | 39637283 | 39637407 |
| chr13 | 39712120 | 39712242 |
| chr13 | 39965468 | 39965598 |
| chr13 | 40148145 | 40148267 |
| chr13 | 40230615 | 40230739 |
| chr13 | 40397154 | 40397280 |
| chr13 | 40454961 | 40455105 |
| chr13 | 40603105 | 40603237 |
| chr13 | 40812243 | 40812383 |
| chr13 | 40902089 | 40902229 |
| chr13 | 41065343 | 41065483 |
| chr13 | 41203863 | 41203989 |
| chr13 | 41376072 | 41376228 |
| chr13 | 41518833 | 41518955 |
| chr13 | 41707182 | 41707320 |
| chr13 | 41810571 | 41810709 |
| chr13 | 42045427 | 42045551 |
| chr13 | 42169992 | 42170134 |
| chr13 | 42349446 | 42349570 |
| chr13 | 42542225 | 42542367 |
| chr13 | 42679745 | 42679869 |
| chr13 | 42751663 | 42751795 |
| chr13 | 42895094 | 42895248 |
| chr13 | 43140034 | 43140172 |
| chr13 | 43280748 | 43280874 |
| chr13 | 43406494 | 43406642 |
| chr13 | 43455322 | 43455466 |
| chr13 | 43691211 | 43691341 |
| chr13 | 43835180 | 43835304 |
| chr13 | 43935904 | 43936036 |
| chr13 | 44181648 | 44181774 |
| chr13 | 44340595 | 44340719 |
| chr13 | 44404765 | 44404887 |
| chr13 | 44618564 | 44618696 |
| chr13 | 44710497 | 44710649 |
| chr13 | 44919619 | 44919747 |
| chr13 | 45093947 | 45094071 |
| chr13 | 45231315 | 45231455 |
| chr13 | 45294479 | 45294621 |
| chr13 | 45545629 | 45545869 |
| chr13 | 45637581 | 45637707 |
| chr13 | 45804384 | 45804636 |
| chr13 | 45947421 | 45947559 |
| chr13 | 46056863 | 46056985 |
| chr13 | 46158360 | 46158490 |
| chr13 | 46366887 | 46367027 |
| chr13 | 46531693 | 46531833 |
| chr13 | 46662572 | 46662714 |
| chr13 | 46800584 | 46800720 |
| chr13 | 46910270 | 46910394 |
| chr13 | 47060747 | 47060871 |
| chr13 | 47221757 | 47221889 |
| chr13 | 47438884 | 47439030 |
| chr13 | 47522912 | 47523042 |
| chr13 | 47674119 | 47674241 |
| chr13 | 47812184 | 47812326 |
| chr13 | 48002548 | 48002674 |
| chr13 | 48240857 | 48240985 |
| chr13 | 48325648 | 48325784 |
| chr13 | 48421144 | 48421270 |
| chr13 | 48630049 | 48630209 |
| chr13 | 48841472 | 48841606 |
| chr13 | 48948531 | 48948655 |
| chr13 | 49038625 | 49038948 |
| chr13 | 49283678 | 49283806 |
| chr13 | 49428062 | 49428186 |
| chr13 | 49504720 | 49504842 |
| chr13 | 49663825 | 49663957 |
| chr13 | 49769269 | 49769397 |
| chr13 | 50019288 | 50019438 |
| chr13 | 50163949 | 50164081 |
| chr13 | 50343830 | 50343968 |
| chr13 | 50446451 | 50446579 |
| chr13 | 50610234 | 50610356 |
| chr13 | 50708814 | 50708972 |
| chr13 | 50898205 | 50898341 |
| chr13 | 51032950 | 51033074 |
| chr13 | 51126819 | 51126963 |
| chr13 | 51287740 | 51287896 |
| chr13 | 51409640 | 51409798 |
| chr13 | 51679944 | 51680084 |
| chr13 | 51782918 | 51783060 |
| chr13 | 51907020 | 51907162 |
| chr13 | 52032892 | 52033048 |
| chr13 | 52219996 | 52220117 |
| chr13 | 52303103 | 52303227 |
| chr13 | 52501177 | 52501301 |
| chr13 | 52693794 | 52693920 |
| chr13 | 52894465 | 52894586 |
| chr13 | 52912828 | 52912970 |
| chr13 | 53190071 | 53190192 |
| chr13 | 53218434 | 53218731 |
| chr13 | 53409120 | 53409250 |
| chr13 | 53648649 | 53648777 |
| chr13 | 53686623 | 53686751 |
| chr13 | 53943255 | 53943385 |
| chr13 | 53995813 | 53995935 |
| chr13 | 54216547 | 54216687 |
| chr13 | 54255726 | 54255848 |
| chr13 | 54419588 | 54419732 |
| chr13 | 54652675 | 54652835 |
| chr13 | 54749014 | 54749138 |
| chr13 | 54963070 | 54963200 |
| chr13 | 55067287 | 55067421 |
| chr13 | 55283829 | 55283953 |
| chr13 | 55384568 | 55384694 |
| chr13 | 55508411 | 55508533 |
| chr13 | 55666557 | 55666681 |
| chr13 | 55753874 | 55754026 |
| chr13 | 55973548 | 55973672 |
| chr13 | 56069251 | 56069393 |
| chr13 | 56249937 | 56250093 |
| chr13 | 56377790 | 56377920 |
| chr13 | 56574438 | 56574578 |
| chr13 | 56705296 | 56705418 |
| chr13 | 56843454 | 56843580 |
| chr13 | 57042735 | 57042877 |
| chr13 | 57108822 | 57108954 |
| chr13 | 57373326 | 57373468 |
| chr13 | 57457340 | 57457490 |
| chr13 | 57650756 | 57650904 |
| chr13 | 57749690 | 57749816 |
| chr13 | 57933707 | 57933847 |
| chr13 | 58020187 | 58020311 |
| chr13 | 58222460 | 58222582 |
| chr13 | 58307927 | 58308063 |
| chr13 | 58500987 | 58501111 |
| chr13 | 58623388 | 58623510 |
| chr13 | 58761277 | 58761409 |
| chr13 | 59037131 | 59037265 |
| chr13 | 59082230 | 59082370 |
| chr13 | 59310874 | 59311004 |
| chr13 | 59454873 | 59454997 |
| chr13 | 59519249 | 59519375 |
| chr13 | 59664362 | 59664488 |
| chr13 | 59898044 | 59898166 |
| chr13 | 60073347 | 60073469 |
| chr13 | 60181382 | 60181622 |
| chr13 | 60289599 | 60289725 |
| chr13 | 60534413 | 60534537 |
| chr13 | 60625893 | 60626041 |
| chr13 | 60794358 | 60794500 |
| chr13 | 60924697 | 60924821 |
| chr13 | 61085798 | 61085920 |
| chr13 | 61298515 | 61298645 |
| chr13 | 61399626 | 61399754 |
| chr13 | 61592819 | 61592941 |
| chr13 | 61661516 | 61661656 |
| chr13 | 61788958 | 61789102 |
| chr13 | 61959697 | 61959827 |
| chr13 | 62116701 | 62116857 |
| chr13 | 62233322 | 62233446 |
| chr13 | 62354492 | 62354624 |
| chr13 | 62632786 | 62632928 |
| chr13 | 62747955 | 62748083 |
| chr13 | 62879572 | 62879722 |
| chr13 | 63023027 | 63023161 |
| chr13 | 63128277 | 63128423 |
| chr13 | 63363000 | 63363146 |
| chr13 | 63403201 | 63403331 |
| chr13 | 63629478 | 63629612 |
| chr13 | 63804771 | 63804895 |
| chr13 | 63868991 | 63869131 |
| chr13 | 64115581 | 64115711 |
| chr13 | 64191427 | 64191555 |
| chr13 | 64420492 | 64420613 |
| chr13 | 64568189 | 64568329 |
| chr13 | 64662659 | 64662799 |
| chr13 | 64901531 | 64901663 |
| chr13 | 65006857 | 65007007 |
| chr13 | 65089419 | 65089545 |
| chr13 | 65239918 | 65240060 |
| chr13 | 65428028 | 65428150 |
| chr13 | 65558193 | 65558323 |
| chr13 | 65675770 | 65675894 |
| chr13 | 65832669 | 65832805 |
| chr13 | 65972205 | 65972351 |
| chr13 | 66150423 | 66150565 |
| chr13 | 66391275 | 66391403 |
| chr13 | 66491656 | 66491786 |
| chr13 | 66611361 | 66611515 |
| chr13 | 66713616 | 66713742 |
| chr13 | 66880948 | 66881090 |
| chr13 | 67126372 | 67126514 |
| chr13 | 67254144 | 67254268 |
| chr13 | 67416551 | 67416673 |
| chr13 | 67526372 | 67526500 |
| chr13 | 67610456 | 67610580 |
| chr13 | 67804007 | 67804353 |
| chr13 | 67930057 | 67930195 |
| chr13 | 68148563 | 68148691 |
| chr13 | 68309721 | 68309843 |
| chr13 | 68500290 | 68500416 |
| chr13 | 68570512 | 68570636 |
| chr13 | 68777549 | 68777671 |
| chr13 | 68921043 | 68921173 |
| chr13 | 69093235 | 69093357 |
| chr13 | 69212738 | 69212862 |
| chr13 | 69336232 | 69336372 |
| chr13 | 69544725 | 69544849 |
| chr13 | 69590543 | 69590681 |
| chr13 | 69738148 | 69738306 |
| chr13 | 69857530 | 69857660 |
| chr13 | 70097615 | 70097737 |
| chr13 | 70261540 | 70261666 |
| chr13 | 70441483 | 70441605 |
| chr13 | 70583609 | 70583749 |
| chr13 | 70640639 | 70640781 |
| chr13 | 70814800 | 70814922 |
| chr13 | 70949156 | 70949280 |
| chr13 | 71161131 | 71161277 |
| chr13 | 71223713 | 71223843 |
| chr13 | 71412929 | 71413059 |
| chr13 | 71523006 | 71523142 |
| chr13 | 71773878 | 71774006 |
| chr13 | 71871690 | 71871820 |
| chr13 | 71981562 | 71981720 |
| chr13 | 72237668 | 72237796 |
| chr13 | 72392634 | 72392766 |
| chr13 | 72450316 | 72450448 |
| chr13 | 72624176 | 72624300 |
| chr13 | 72833446 | 72833576 |
| chr13 | 72864492 | 72864616 |
| chr13 | 73078437 | 73078573 |
| chr13 | 73214059 | 73214227 |
| chr13 | 73387637 | 73387787 |
| chr13 | 73583504 | 73583626 |
| chr13 | 73693748 | 73693872 |
| chr13 | 73892423 | 73892547 |
| chr13 | 74031254 | 74031380 |
| chr13 | 74157258 | 74157390 |
| chr13 | 74249775 | 74249899 |
| chr13 | 74480867 | 74480993 |
| chr13 | 74508737 | 74508883 |
| chr13 | 74796009 | 74796153 |
| chr13 | 74898605 | 74898743 |
| chr13 | 75086801 | 75086931 |
| chr13 | 75186874 | 75187018 |
| chr13 | 75261582 | 75261706 |
| chr13 | 75451356 | 75451480 |
| chr13 | 75575558 | 75575724 |
| chr13 | 75730634 | 75730758 |
| chr13 | 75911585 | 75911719 |
| chr13 | 76084930 | 76085070 |
| chr13 | 76255993 | 76256115 |
| chr13 | 76431436 | 76431588 |
| chr13 | 76498916 | 76499054 |
| chr13 | 76668657 | 76668795 |
| chr13 | 76836006 | 76836156 |
| chr13 | 76935040 | 76935166 |
| chr13 | 77072621 | 77072743 |
| chr13 | 77238437 | 77238561 |
| chr13 | 77440003 | 77440163 |
| chr13 | 77594246 | 77594384 |
| chr13 | 77699007 | 77699139 |
| chr13 | 77834212 | 77834336 |
| chr13 | 78000442 | 78000584 |
| chr13 | 78178097 | 78178231 |
| chr13 | 78392566 | 78392722 |
| chr13 | 78465204 | 78465494 |
| chr13 | 78604797 | 78604919 |
| chr13 | 78829954 | 78830088 |
| chr13 | 78939905 | 78940035 |
| chr13 | 79042090 | 79042228 |
| chr13 | 79155803 | 79155931 |
| chr13 | 79392032 | 79392166 |
| chr13 | 79508401 | 79508525 |
| chr13 | 79677662 | 79677786 |
| chr13 | 79880829 | 79880969 |
| chr13 | 79962638 | 79962766 |
| chr13 | 80091707 | 80091841 |
| chr13 | 80324871 | 80324995 |
| chr13 | 80489198 | 80489334 |
| chr13 | 80608822 | 80608946 |
| chr13 | 80757268 | 80757392 |
| chr13 | 80883556 | 80883730 |
| chr13 | 81049982 | 81050110 |
| chr13 | 81243995 | 81244137 |
| chr13 | 81353440 | 81353582 |
| chr13 | 81520592 | 81520734 |
| chr13 | 81598697 | 81598823 |
| chr13 | 81816938 | 81817088 |
| chr13 | 81948839 | 81948975 |
| chr13 | 82117476 | 82117614 |
| chr13 | 82168209 | 82168337 |
| chr13 | 82372197 | 82372321 |
| chr13 | 82499727 | 82499857 |
| chr13 | 82677138 | 82677274 |
| chr13 | 82845403 | 82845537 |
| chr13 | 82930990 | 82931118 |
| chr13 | 83071606 | 83071738 |
| chr13 | 83229094 | 83229228 |
| chr13 | 83484615 | 83484757 |
| chr13 | 83509978 | 83510124 |
| chr13 | 83753336 | 83753462 |
| chr13 | 83942832 | 83942962 |
| chr13 | 84090521 | 84090645 |
| chr13 | 84131061 | 84131185 |
| chr13 | 84368749 | 84368873 |
| chr13 | 84456677 | 84456823 |
| chr13 | 84580908 | 84581032 |
| chr13 | 84766976 | 84767126 |
| chr13 | 84853562 | 84853736 |
| chr13 | 85071821 | 85071945 |
| chr13 | 85251579 | 85251721 |
| chr13 | 85384080 | 85384204 |
| chr13 | 85452431 | 85452567 |
| chr13 | 85749583 | 85749719 |
| chr13 | 85809558 | 85809680 |
| chr13 | 85947489 | 85947631 |
| chr13 | 86070526 | 86070650 |
| chr13 | 86250613 | 86250735 |
| chr13 | 86461565 | 86461689 |
| chr13 | 86517137 | 86517283 |
| chr13 | 86695430 | 86695558 |
| chr13 | 86917378 | 86917502 |
| chr13 | 87096260 | 87096384 |
| chr13 | 87116415 | 87116551 |
| chr13 | 87270060 | 87270192 |
| chr13 | 87459668 | 87459792 |
| chr13 | 87658934 | 87659058 |
| chr13 | 87825723 | 87825983 |
| chr13 | 87950232 | 87950368 |
| chr13 | 88108768 | 88108890 |
| chr13 | 88226499 | 88226625 |
| chr13 | 88312273 | 88312417 |
| chr13 | 88553694 | 88553826 |
| chr13 | 88608765 | 88608907 |
| chr13 | 88757145 | 88757285 |
| chr13 | 89000630 | 89000754 |
| chr13 | 89086350 | 89086476 |
| chr13 | 89325315 | 89325439 |
| chr13 | 89473003 | 89473135 |
| chr13 | 89525110 | 89525256 |
| chr13 | 89770621 | 89770745 |
| chr13 | 89891070 | 89891196 |
| chr13 | 90008548 | 90008688 |
| chr13 | 90139239 | 90139369 |
| chr13 | 90281254 | 90281386 |
| chr13 | 90422848 | 90422994 |
| chr13 | 90681528 | 90681654 |
| chr13 | 90782411 | 90782533 |
| chr13 | 90936122 | 90936246 |
| chr13 | 91049536 | 91049670 |
| chr13 | 91203607 | 91203731 |
| chr13 | 91398174 | 91398300 |
| chr13 | 91590447 | 91590589 |
| chr13 | 91725405 | 91725535 |
| chr13 | 91829858 | 91829996 |
| chr13 | 92011772 | 92011908 |
| chr13 | 92072039 | 92072163 |
| chr13 | 92210582 | 92210708 |
| chr13 | 92381452 | 92381586 |
| chr13 | 92617495 | 92617631 |
| chr13 | 92774776 | 92774904 |
| chr13 | 92939533 | 92939661 |
| chr13 | 92994435 | 92994561 |
| chr13 | 93219546 | 93219690 |
| chr13 | 93264182 | 93264312 |
| chr13 | 93468746 | 93468880 |
| chr13 | 93671902 | 93672044 |
| chr13 | 93765306 | 93765428 |
| chr13 | 93947043 | 93947165 |
| chr13 | 94034050 | 94034174 |
| chr13 | 94270589 | 94270713 |
| chr13 | 94397619 | 94397759 |
| chr13 | 94589118 | 94589250 |
| chr13 | 94662076 | 94662200 |
| chr13 | 94815496 | 94815659 |
| chr13 | 94965126 | 94965268 |
| chr13 | 95196526 | 95196650 |
| chr13 | 95348638 | 95348764 |
| chr13 | 95415499 | 95415623 |
| chr13 | 95630797 | 95630939 |
| chr13 | 95736952 | 95737076 |
| chr13 | 95835628 | 95835766 |
| chr13 | 95966781 | 95966935 |
| chr13 | 96109194 | 96109322 |
| chr13 | 96295147 | 96295299 |
| chr13 | 96534549 | 96534691 |
| chr13 | 96647905 | 96648027 |
| chr13 | 96845505 | 96845627 |
| chr13 | 96891086 | 96891212 |
| chr13 | 97040463 | 97040601 |
| chr13 | 97264927 | 97265110 |
| chr13 | 97344696 | 97344826 |
| chr13 | 97525540 | 97525688 |
| chr13 | 97647126 | 97647274 |
| chr13 | 97770377 | 97770517 |
| chr13 | 97953385 | 97953511 |
| chr13 | 98115130 | 98115268 |
| chr13 | 98216535 | 98216665 |
| chr13 | 98485564 | 98485700 |
| chr13 | 98576645 | 98576769 |
| chr13 | 98680600 | 98680748 |
| chr13 | 98928433 | 98928569 |
| chr13 | 99045270 | 99045408 |
| chr13 | 99234279 | 99234401 |
| chr13 | 99304473 | 99304609 |
| chr13 | 99431269 | 99431395 |
| chr13 | 99685242 | 99685388 |
| chr13 | 99841753 | 99841891 |
| chr13 | 99957941 | 99958069 |
| chr13 | 100017069 | 100017193 |
| chr13 | 100216821 | 100216967 |
| chr13 | 100392317 | 100392441 |
| chr13 | 100527663 | 100527787 |
| chr13 | 100638517 | 100638787 |
| chr13 | 100796346 | 100796476 |
| chr13 | 100950362 | 100950516 |
| chr13 | 101095773 | 101095897 |
| chr13 | 101227441 | 101227573 |
| chr13 | 101417899 | 101418041 |
| chr13 | 101526731 | 101526861 |
| chr13 | 101660444 | 101660584 |
| chr13 | 101845911 | 101846057 |
| chr13 | 102081982 | 102082132 |
| chr13 | 102180201 | 102180343 |
| chr13 | 102332019 | 102332157 |
| chr13 | 102429577 | 102429753 |
| chr13 | 102640162 | 102640302 |
| chr13 | 102770777 | 102770905 |
| chr13 | 102981823 | 102981967 |
| chr13 | 103067117 | 103067247 |
| chr13 | 103236229 | 103236369 |
| chr13 | 103371720 | 103371878 |
| chr13 | 103466924 | 103467068 |
| chr13 | 103697648 | 103697792 |
| chr13 | 103880562 | 103880710 |
| chr13 | 103952187 | 103952321 |
| chr13 | 104135100 | 104135222 |
| chr13 | 104315265 | 104315415 |
| chr13 | 104424188 | 104424312 |
| chr13 | 104591622 | 104591750 |
| chr13 | 104659438 | 104659572 |
| chr13 | 104819842 | 104820004 |
| chr13 | 104988835 | 104988973 |
| chr13 | 105144396 | 105144520 |
| chr13 | 105399482 | 105399614 |
| chr13 | 105521689 | 105521821 |
| chr13 | 105581792 | 105581928 |
| chr13 | 105730486 | 105730618 |
| chr13 | 105945406 | 105945548 |
| chr13 | 106061846 | 106061982 |
| chr13 | 106196261 | 106196385 |
| chr13 | 106408138 | 106408274 |
| chr13 | 106485714 | 106485838 |
| chr13 | 106746822 | 106746950 |
| chr13 | 106829730 | 106829864 |
| chr13 | 106911060 | 106911182 |
| chr13 | 107099338 | 107099478 |
| chr13 | 107214616 | 107214740 |
| chr13 | 107424233 | 107424359 |
| chr13 | 107534731 | 107534855 |
| chr13 | 107675273 | 107675419 |
| chr13 | 107918688 | 107918820 |
| chr13 | 108000051 | 108000173 |
| chr13 | 108146761 | 108146889 |
| chr13 | 108253698 | 108253822 |
| chr13 | 108524165 | 108524311 |
| chr13 | 108692948 | 108693072 |
| chr13 | 108709913 | 108710035 |
| chr13 | 108879396 | 108879520 |
| chr13 | 109057049 | 109057185 |
| chr13 | 109271020 | 109271150 |
| chr13 | 109364272 | 109364396 |
| chr13 | 109510191 | 109510333 |
| chr13 | 109722589 | 109722725 |
| chr13 | 109799730 | 109799856 |
| chr13 | 109932644 | 109932770 |
| chr13 | 110180673 | 110180811 |
| chr13 | 110235824 | 110235982 |
| chr13 | 110456174 | 110456314 |
| chr13 | 110585090 | 110585232 |
| chr13 | 110656107 | 110656237 |
| chr13 | 110823278 | 110823428 |
| chr13 | 111040724 | 111040846 |
| chr13 | 111243678 | 111243824 |
| chr13 | 111373308 | 111373448 |
| chr13 | 111439768 | 111439918 |
| chr13 | 111673445 | 111673581 |
| chr13 | 111749072 | 111749204 |
| chr13 | 111902828 | 111902952 |
| chr13 | 112009346 | 112009498 |
| chr13 | 112227300 | 112227430 |
| chr13 | 112352620 | 112352742 |
| chr13 | 112582262 | 112582384 |
| chr13 | 112678402 | 112678528 |
| chr13 | 112875361 | 112875483 |
| chr13 | 112969461 | 112969714 |
| chr13 | 113139502 | 113139626 |
| chr13 | 113238857 | 113238981 |
| chr13 | 113421998 | 113422122 |
| chr13 | 113625320 | 113625444 |
| chr13 | 113713857 | 113713983 |
| chr13 | 113944567 | 113944713 |
| chr13 | 114074656 | 114074780 |
| chr13 | 114189560 | 114189706 |
| chr13 | 114277165 | 114277289 |
| chr13 | 114447909 | 114448036 |
| chr13 | 114600349 | 114600471 |
| chr13 | 114752722 | 114752866 |
| chr13 | 114998028 | 114998154 |
| chr13 | 115025310 | 115025432 |
| chr14 | 19096972 | 19097100 |
| chr14 | 19193401 | 19193523 |
| chr14 | 19372097 | 19372271 |
| chr14 | 19793418 | 19793539 |
| chr14 | 19812711 | 19812885 |
| chr14 | 19970877 | 19971003 |
| chr14 | 20187739 | 20187913 |
| chr14 | 20316441 | 20316565 |
| chr14 | 20518611 | 20518767 |
| chr14 | 20677555 | 20677679 |
| chr14 | 20746415 | 20746540 |
| chr14 | 20971694 | 20971832 |
| chr14 | 21090443 | 21090621 |
| chr14 | 21188597 | 21188735 |
| chr14 | 21433309 | 21433433 |
| chr14 | 21592051 | 21592177 |
| chr14 | 21603981 | 21604125 |
| chr14 | 21852964 | 21853243 |
| chr14 | 21903010 | 21903137 |
| chr14 | 22127569 | 22127699 |
| chr14 | 22271088 | 22271212 |
| chr14 | 22461332 | 22461458 |
| chr14 | 22530317 | 22530455 |
| chr14 | 22764755 | 22764907 |
| chr14 | 22838775 | 22838903 |
| chr14 | 23032742 | 23032866 |
| chr14 | 23168209 | 23168333 |
| chr14 | 23363447 | 23363571 |
| chr14 | 23482368 | 23482492 |
| chr14 | 23656827 | 23656961 |
| chr14 | 23812161 | 23812287 |
| chr14 | 23926311 | 23926447 |
| chr14 | 24141417 | 24141539 |
| chr14 | 24207446 | 24207572 |
| chr14 | 24406852 | 24407073 |
| chr14 | 24591828 | 24591954 |
| chr14 | 24764987 | 24765111 |
| chr14 | 24941879 | 24942031 |
| chr14 | 25110029 | 25110155 |
| chr14 | 25249776 | 25249902 |
| chr14 | 25383319 | 25383461 |
| chr14 | 25554541 | 25554693 |
| chr14 | 25770340 | 25770480 |
| chr14 | 25845999 | 25846127 |
| chr14 | 25970625 | 25970749 |
| chr14 | 26138284 | 26138424 |
| chr14 | 26259331 | 26259457 |
| chr14 | 26434054 | 26434180 |
| chr14 | 26626347 | 26626473 |
| chr14 | 26756786 | 26756914 |
| chr14 | 26912133 | 26912271 |
| chr14 | 27009125 | 27009249 |
| chr14 | 27244306 | 27244430 |
| chr14 | 27402697 | 27402855 |
| chr14 | 27465615 | 27465749 |
| chr14 | 27627256 | 27627400 |
| chr14 | 27791203 | 27791349 |
| chr14 | 28015163 | 28015311 |
| chr14 | 28105974 | 28106108 |
| chr14 | 28268978 | 28269128 |
| chr14 | 28324239 | 28324360 |
| chr14 | 28444740 | 28444868 |
| chr14 | 28582331 | 28582455 |
| chr14 | 28712768 | 28712896 |
| chr14 | 28918696 | 28918834 |
| chr14 | 29066612 | 29066744 |
| chr14 | 29169863 | 29169993 |
| chr14 | 29243898 | 29244102 |
| chr14 | 29438509 | 29438641 |
| chr14 | 29546932 | 29547056 |
| chr14 | 29814421 | 29814549 |
| chr14 | 29905676 | 29905822 |
| chr14 | 30137353 | 30137477 |
| chr14 | 30242994 | 30243119 |
| chr14 | 30294820 | 30294946 |
| chr14 | 30491008 | 30491154 |
| chr14 | 30666321 | 30666451 |
| chr14 | 30807834 | 30807966 |
| chr14 | 30901679 | 30901815 |
| chr14 | 31161353 | 31161503 |
| chr14 | 31196938 | 31197060 |
| chr14 | 31386346 | 31386480 |
| chr14 | 31501437 | 31501561 |
| chr14 | 31737462 | 31737608 |
| chr14 | 31807284 | 31807428 |
| chr14 | 32075694 | 32075824 |
| chr14 | 32231855 | 32231981 |
| chr14 | 32385843 | 32385973 |
| chr14 | 32473980 | 32474108 |
| chr14 | 32630444 | 32630718 |
| chr14 | 32756039 | 32756179 |
| chr14 | 32973829 | 32973951 |
| chr14 | 33076485 | 33076617 |
| chr14 | 33157980 | 33158120 |
| chr14 | 33366266 | 33366396 |
| chr14 | 33516672 | 33516796 |
| chr14 | 33610410 | 33610552 |
| chr14 | 33828621 | 33828765 |
| chr14 | 34017658 | 34017808 |
| chr14 | 34136366 | 34136490 |
| chr14 | 34201637 | 34201759 |
| chr14 | 34440948 | 34441080 |
| chr14 | 34601257 | 34601395 |
| chr14 | 34707568 | 34707690 |
| chr14 | 34857464 | 34857604 |
| chr14 | 35067458 | 35067600 |
| chr14 | 35116199 | 35116337 |
| chr14 | 35350234 | 35350374 |
| chr14 | 35417142 | 35417266 |
| chr14 | 35619540 | 35619672 |
| chr14 | 35716080 | 35716204 |
| chr14 | 35900644 | 35900766 |
| chr14 | 36118288 | 36118436 |
| chr14 | 36209419 | 36209541 |
| chr14 | 36393832 | 36393960 |
| chr14 | 36451884 | 36452010 |
| chr14 | 36597860 | 36597982 |
| chr14 | 36825085 | 36825241 |
| chr14 | 37019639 | 37019791 |
| chr14 | 37121067 | 37121188 |
| chr14 | 37341598 | 37341742 |
| chr14 | 37452422 | 37452546 |
| chr14 | 37635643 | 37635775 |
| chr14 | 37785617 | 37785741 |
| chr14 | 37850122 | 37850268 |
| chr14 | 38023233 | 38023367 |
| chr14 | 38123064 | 38123214 |
| chr14 | 38281179 | 38281303 |
| chr14 | 38457098 | 38457236 |
| chr14 | 38580034 | 38580168 |
| chr14 | 38820260 | 38820396 |
| chr14 | 38865900 | 38866050 |
| chr14 | 39040325 | 39040449 |
| chr14 | 39246210 | 39246334 |
| chr14 | 39315919 | 39316043 |
| chr14 | 39470372 | 39470504 |
| chr14 | 39603974 | 39604096 |
| chr14 | 39781428 | 39781560 |
| chr14 | 39982373 | 39982515 |
| chr14 | 40067206 | 40067336 |
| chr14 | 40307662 | 40307808 |
| chr14 | 40408238 | 40408370 |
| chr14 | 40640142 | 40640276 |
| chr14 | 40726465 | 40726601 |
| chr14 | 40872052 | 40872186 |
| chr14 | 40996055 | 40996211 |
| chr14 | 41153921 | 41154053 |
| chr14 | 41371996 | 41372136 |
| chr14 | 41528005 | 41528131 |
| chr14 | 41577680 | 41577836 |
| chr14 | 41702629 | 41702763 |
| chr14 | 41847194 | 41847334 |
| chr14 | 42024311 | 42024433 |
| chr14 | 42160596 | 42160720 |
| chr14 | 42308872 | 42309012 |
| chr14 | 42498552 | 42498698 |
| chr14 | 42719676 | 42719802 |
| chr14 | 42877055 | 42877181 |
| chr14 | 43013315 | 43013439 |
| chr14 | 43172226 | 43172350 |
| chr14 | 43257925 | 43258047 |
| chr14 | 43435657 | 43435781 |
| chr14 | 43596389 | 43596513 |
| chr14 | 43730715 | 43730861 |
| chr14 | 43868675 | 43868803 |
| chr14 | 44045169 | 44045309 |
| chr14 | 44166813 | 44166939 |
| chr14 | 44347426 | 44347552 |
| chr14 | 44433104 | 44433248 |
| chr14 | 44547805 | 44547937 |
| chr14 | 44814427 | 44814557 |
| chr14 | 44874371 | 44874509 |
| chr14 | 45035193 | 45035317 |
| chr14 | 45157233 | 45157361 |
| chr14 | 45311995 | 45312119 |
| chr14 | 45584852 | 45584972 |
| chr14 | 45720553 | 45720687 |
| chr14 | 45857805 | 45857927 |
| chr14 | 45986921 | 45987079 |
| chr14 | 46095802 | 46095940 |
| chr14 | 46235424 | 46235560 |
| chr14 | 46444112 | 46444250 |
| chr14 | 46574355 | 46574491 |
| chr14 | 46728741 | 46728871 |
| chr14 | 46848077 | 46848219 |
| chr14 | 47071781 | 47071907 |
| chr14 | 47219347 | 47219469 |
| chr14 | 47358438 | 47358562 |
| chr14 | 47442572 | 47442724 |
| chr14 | 47562672 | 47562796 |
| chr14 | 47795933 | 47796055 |
| chr14 | 47860588 | 47860710 |
| chr14 | 48074373 | 48074519 |
| chr14 | 48150045 | 48150177 |
| chr14 | 48404490 | 48404614 |
| chr14 | 48484726 | 48484864 |
| chr14 | 48676347 | 48676469 |
| chr14 | 48769124 | 48769260 |
| chr14 | 48937715 | 48937849 |
| chr14 | 49179380 | 49179512 |
| chr14 | 49310078 | 49310204 |
| chr14 | 49430969 | 49431093 |
| chr14 | 49508779 | 49508929 |
| chr14 | 49725696 | 49725824 |
| chr14 | 49905352 | 49905476 |
| chr14 | 49968806 | 49968934 |
| chr14 | 50188334 | 50188596 |
| chr14 | 50378782 | 50378903 |
| chr14 | 50401128 | 50401252 |
| chr14 | 50622173 | 50622331 |
| chr14 | 50696726 | 50696856 |
| chr14 | 50877895 | 50878021 |
| chr14 | 51125188 | 51125310 |
| chr14 | 51176677 | 51176827 |
| chr14 | 51395043 | 51395169 |
| chr14 | 51485337 | 51485465 |
| chr14 | 51597354 | 51597480 |
| chr14 | 51852972 | 51853096 |
| chr14 | 52039692 | 52039818 |
| chr14 | 52086348 | 52086476 |
| chr14 | 52302100 | 52302242 |
| chr14 | 52420825 | 52420977 |
| chr14 | 52553869 | 52553999 |
| chr14 | 52714807 | 52714931 |
| chr14 | 52829995 | 52830141 |
| chr14 | 53023517 | 53023655 |
| chr14 | 53189667 | 53189817 |
| chr14 | 53244626 | 53244750 |
| chr14 | 53505851 | 53505977 |
| chr14 | 53562560 | 53562684 |
| chr14 | 53809562 | 53809702 |
| chr14 | 53853483 | 53853653 |
| chr14 | 53996054 | 53996198 |
| chr14 | 54238937 | 54239085 |
| chr14 | 54425627 | 54425987 |
| chr14 | 54551416 | 54551558 |
| chr14 | 54674284 | 54674418 |
| chr14 | 54755694 | 54755818 |
| chr14 | 55037112 | 55037236 |
| chr14 | 55114489 | 55114631 |
| chr14 | 55325523 | 55325661 |
| chr14 | 55343927 | 55344071 |
| chr14 | 55594567 | 55594713 |
| chr14 | 55775378 | 55775516 |
| chr14 | 55845119 | 55845267 |
| chr14 | 56042919 | 56043063 |
| chr14 | 56093876 | 56094012 |
| chr14 | 56341653 | 56341777 |
| chr14 | 56451879 | 56452011 |
| chr14 | 56566870 | 56567014 |
| chr14 | 56767941 | 56768065 |
| chr14 | 56880179 | 56880305 |
| chr14 | 57126691 | 57126815 |
| chr14 | 57245153 | 57245285 |
| chr14 | 57297477 | 57297621 |
| chr14 | 57518471 | 57518623 |
| chr14 | 57674981 | 57675129 |
| chr14 | 57876430 | 57876556 |
| chr14 | 57998370 | 57998528 |
| chr14 | 58116276 | 58116418 |
| chr14 | 58240200 | 58240340 |
| chr14 | 58343542 | 58343686 |
| chr14 | 58565252 | 58565392 |
| chr14 | 58750866 | 58750988 |
| chr14 | 58814989 | 58815111 |
| chr14 | 59051594 | 59051722 |
| chr14 | 59212082 | 59212226 |
| chr14 | 59277526 | 59277672 |
| chr14 | 59415947 | 59416085 |
| chr14 | 59554537 | 59554685 |
| chr14 | 59833120 | 59833244 |
| chr14 | 59899964 | 59900102 |
| chr14 | 60122201 | 60122327 |
| chr14 | 60263862 | 60263988 |
| chr14 | 60331698 | 60331842 |
| chr14 | 60565191 | 60565315 |
| chr14 | 60700837 | 60700979 |
| chr14 | 60777197 | 60777327 |
| chr14 | 60997074 | 60997200 |
| chr14 | 61187465 | 61187788 |
| chr14 | 61202175 | 61202430 |
| chr14 | 61419041 | 61419169 |
| chr14 | 61516155 | 61516301 |
| chr14 | 61727571 | 61727697 |
| chr14 | 61940856 | 61940982 |
| chr14 | 61961326 | 61961470 |
| chr14 | 62214535 | 62214675 |
| chr14 | 62364049 | 62364173 |
| chr14 | 62436042 | 62436172 |
| chr14 | 62636227 | 62636361 |
| chr14 | 62826555 | 62826685 |
| chr14 | 62938333 | 62938455 |
| chr14 | 63096033 | 63096191 |
| chr14 | 63179006 | 63179140 |
| chr14 | 63412407 | 63412529 |
| chr14 | 63568096 | 63568224 |
| chr14 | 63710341 | 63710495 |
| chr14 | 63837725 | 63837847 |
| chr14 | 64029745 | 64029869 |
| chr14 | 64143472 | 64143767 |
| chr14 | 64226230 | 64226483 |
| chr14 | 64442915 | 64443069 |
| chr14 | 64609536 | 64609692 |
| chr14 | 64696949 | 64697087 |
| chr14 | 64871158 | 64871284 |
| chr14 | 65072141 | 65072267 |
| chr14 | 65159597 | 65159719 |
| chr14 | 65353099 | 65353237 |
| chr14 | 65535331 | 65535473 |
| chr14 | 65566659 | 65566783 |
| chr14 | 65742379 | 65742503 |
| chr14 | 65946686 | 65946816 |
| chr14 | 65998005 | 65998139 |
| chr14 | 66191692 | 66191824 |
| chr14 | 66345317 | 66345439 |
| chr14 | 66572203 | 66572335 |
| chr14 | 66604356 | 66604480 |
| chr14 | 66841706 | 66841832 |
| chr14 | 66915375 | 66915529 |
| chr14 | 67078829 | 67078953 |
| chr14 | 67296671 | 67296801 |
| chr14 | 67470637 | 67470765 |
| chr14 | 67539623 | 67539747 |
| chr14 | 67738019 | 67738155 |
| chr14 | 67802910 | 67803058 |
| chr14 | 68015615 | 68015739 |
| chr14 | 68213226 | 68213368 |
| chr14 | 68362151 | 68362319 |
| chr14 | 68498850 | 68498976 |
| chr14 | 68583773 | 68583903 |
| chr14 | 68713818 | 68713946 |
| chr14 | 68944591 | 68944739 |
| chr14 | 69039538 | 69039664 |
| chr14 | 69178913 | 69179051 |
| chr14 | 69335349 | 69335471 |
| chr14 | 69537157 | 69537293 |
| chr14 | 69599510 | 69599644 |
| chr14 | 69801776 | 69801920 |
| chr14 | 69903333 | 69903489 |
| chr14 | 70047646 | 70047796 |
| chr14 | 70316411 | 70316553 |
| chr14 | 70371613 | 70371753 |
| chr14 | 70523613 | 70523761 |
| chr14 | 70738498 | 70738620 |
| chr14 | 70894547 | 70894681 |
| chr14 | 70992619 | 70992763 |
| chr14 | 71117187 | 71117311 |
| chr14 | 71296596 | 71296722 |
| chr14 | 71464338 | 71464470 |
| chr14 | 71654947 | 71655071 |
| chr14 | 71729350 | 71729472 |
| chr14 | 71864361 | 71864483 |
| chr14 | 72089599 | 72089889 |
| chr14 | 72289585 | 72289707 |
| chr14 | 72438337 | 72438463 |
| chr14 | 72572093 | 72572219 |
| chr14 | 72680226 | 72680396 |
| chr14 | 72816327 | 72816463 |
| chr14 | 72960491 | 72960613 |
| chr14 | 73070575 | 73070707 |
| chr14 | 73273458 | 73273582 |
| chr14 | 73473757 | 73473891 |
| chr14 | 73579341 | 73579469 |
| chr14 | 73767124 | 73767262 |
| chr14 | 73810133 | 73810277 |
| chr14 | 74035035 | 74035162 |
| chr14 | 74162798 | 74162922 |
| chr14 | 74377582 | 74377726 |
| chr14 | 74501939 | 74502069 |
| chr14 | 74674816 | 74674956 |
| chr14 | 74808019 | 74808165 |
| chr14 | 74965725 | 74965849 |
| chr14 | 75049383 | 75049515 |
| chr14 | 75261551 | 75261679 |
| chr14 | 75443065 | 75443189 |
| chr14 | 75546493 | 75546643 |
| chr14 | 75671589 | 75671735 |
| chr14 | 75807537 | 75807661 |
| chr14 | 75906243 | 75906387 |
| chr14 | 76167788 | 76167918 |
| chr14 | 76298618 | 76298740 |
| chr14 | 76463254 | 76463388 |
| chr14 | 76564328 | 76564478 |
| chr14 | 76762738 | 76762866 |
| chr14 | 76934788 | 76934926 |
| chr14 | 77058451 | 77058585 |
| chr14 | 77199642 | 77199778 |
| chr14 | 77274907 | 77275055 |
| chr14 | 77508392 | 77508514 |
| chr14 | 77578301 | 77578453 |
| chr14 | 77700150 | 77700292 |
| chr14 | 77881082 | 77881208 |
| chr14 | 78040344 | 78040468 |
| chr14 | 78291170 | 78291300 |
| chr14 | 78428111 | 78428235 |
| chr14 | 78510258 | 78510390 |
| chr14 | 78734707 | 78734853 |
| chr14 | 78869384 | 78869534 |
| chr14 | 78961994 | 78962124 |
| chr14 | 79078946 | 79079122 |
| chr14 | 79268242 | 79268380 |
| chr14 | 79428396 | 79428536 |
| chr14 | 79495108 | 79495262 |
| chr14 | 79704984 | 79705122 |
| chr14 | 79815452 | 79815578 |
| chr14 | 79981210 | 79981338 |
| chr14 | 80170780 | 80170918 |
| chr14 | 80371496 | 80371650 |
| chr14 | 80449707 | 80449837 |
| chr14 | 80597362 | 80597492 |
| chr14 | 80754514 | 80754636 |
| chr14 | 80959555 | 80959693 |
| chr14 | 81085266 | 81085438 |
| chr14 | 81159169 | 81159299 |
| chr14 | 81438057 | 81438181 |
| chr14 | 81524367 | 81524493 |
| chr14 | 81613378 | 81613506 |
| chr14 | 81810907 | 81811029 |
| chr14 | 81996279 | 81996435 |
| chr14 | 82092831 | 82092985 |
| chr14 | 82246552 | 82246702 |
| chr14 | 82394930 | 82395078 |
| chr14 | 82541298 | 82541422 |
| chr14 | 82724973 | 82725115 |
| chr14 | 82849280 | 82849418 |
| chr14 | 83020210 | 83020346 |
| chr14 | 83207199 | 83207321 |
| chr14 | 83363360 | 83363494 |
| chr14 | 83406648 | 83406778 |
| chr14 | 83676435 | 83676561 |
| chr14 | 83812965 | 83813109 |
| chr14 | 83879474 | 83879598 |
| chr14 | 84023631 | 84023755 |
| chr14 | 84180048 | 84180186 |
| chr14 | 84432260 | 84432382 |
| chr14 | 84585624 | 84585748 |
| chr14 | 84637422 | 84637562 |
| chr14 | 84752172 | 84752296 |
| chr14 | 84989743 | 84989901 |
| chr14 | 85175248 | 85175402 |
| chr14 | 85331286 | 85331416 |
| chr14 | 85392293 | 85392427 |
| chr14 | 85582989 | 85583113 |
| chr14 | 85784540 | 85784676 |
| chr14 | 85828500 | 85828624 |
| chr14 | 86031084 | 86031226 |
| chr14 | 86176305 | 86176429 |
| chr14 | 86351276 | 86351428 |
| chr14 | 86406521 | 86406647 |
| chr14 | 86590549 | 86590695 |
| chr14 | 86801488 | 86801612 |
| chr14 | 86991309 | 86991457 |
| chr14 | 87025093 | 87025251 |
| chr14 | 87179176 | 87179318 |
| chr14 | 87358210 | 87358336 |
| chr14 | 87481373 | 87481511 |
| chr14 | 87606271 | 87606397 |
| chr14 | 87849590 | 87849720 |
| chr14 | 88034803 | 88034925 |
| chr14 | 88144510 | 88144634 |
| chr14 | 88216226 | 88216354 |
| chr14 | 88353446 | 88353570 |
| chr14 | 88498032 | 88498174 |
| chr14 | 88745474 | 88745614 |
| chr14 | 88921636 | 88921768 |
| chr14 | 89025288 | 89025444 |
| chr14 | 89226828 | 89226954 |
| chr14 | 89260527 | 89260669 |
| chr14 | 89455457 | 89455593 |
| chr14 | 89564900 | 89565048 |
| chr14 | 89745559 | 89745689 |
| chr14 | 89961479 | 89961619 |
| chr14 | 90029382 | 90029508 |
| chr14 | 90229044 | 90229174 |
| chr14 | 90440609 | 90440739 |
| chr14 | 90584243 | 90584369 |
| chr14 | 90716736 | 90716860 |
| chr14 | 90836281 | 90836413 |
| chr14 | 90933954 | 90934078 |
| chr14 | 91062757 | 91062897 |
| chr14 | 91235297 | 91235419 |
| chr14 | 91378732 | 91378860 |
| chr14 | 91524342 | 91524476 |
| chr14 | 91756137 | 91756285 |
| chr14 | 91896444 | 91896600 |
| chr14 | 91984460 | 91984614 |
| chr14 | 92184424 | 92184548 |
| chr14 | 92346141 | 92346265 |
| chr14 | 92525397 | 92525527 |
| chr14 | 92560798 | 92560952 |
| chr14 | 92816251 | 92816385 |
| chr14 | 92868394 | 92868518 |
| chr14 | 93101476 | 93101616 |
| chr14 | 93164020 | 93164166 |
| chr14 | 93298967 | 93299099 |
| chr14 | 93587375 | 93587499 |
| chr14 | 93619472 | 93619604 |
| chr14 | 93744043 | 93744173 |
| chr14 | 93997802 | 93997930 |
| chr14 | 94099748 | 94099870 |
| chr14 | 94300904 | 94301030 |
| chr14 | 94410562 | 94410696 |
| chr14 | 94562248 | 94562378 |
| chr14 | 94670060 | 94670194 |
| chr14 | 94801308 | 94801438 |
| chr14 | 94969640 | 94969772 |
| chr14 | 95189287 | 95189415 |
| chr14 | 95365693 | 95365847 |
| chr14 | 95446089 | 95446237 |
| chr14 | 95575378 | 95575500 |
| chr14 | 95764230 | 95764380 |
| chr14 | 95911559 | 95911683 |
| chr14 | 96001988 | 96002112 |
| chr14 | 96285034 | 96285158 |
| chr14 | 96371884 | 96372008 |
| chr14 | 96482805 | 96482935 |
| chr14 | 96605273 | 96605413 |
| chr14 | 96797435 | 96797559 |
| chr14 | 96933039 | 96933201 |
| chr14 | 97134635 | 97134759 |
| chr14 | 97343233 | 97343371 |
| chr14 | 97453348 | 97453496 |
| chr14 | 97594134 | 97594256 |
| chr14 | 97763020 | 97763170 |
| chr14 | 97889208 | 97889334 |
| chr14 | 98051133 | 98051374 |
| chr14 | 98197838 | 98197972 |
| chr14 | 98368169 | 98368305 |
| chr14 | 98411278 | 98411418 |
| chr14 | 98691376 | 98691524 |
| chr14 | 98761931 | 98762055 |
| chr14 | 98959618 | 98959746 |
| chr14 | 99032538 | 99032660 |
| chr14 | 99213363 | 99213499 |
| chr14 | 99311652 | 99311794 |
| chr14 | 99481220 | 99481360 |
| chr14 | 99624026 | 99624182 |
| chr14 | 99835362 | 99835514 |
| chr14 | 99914803 | 99914969 |
| chr14 | 100051972 | 100052094 |
| chr14 | 100322660 | 100322806 |
| chr14 | 100448357 | 100448491 |
| chr14 | 100495176 | 100495318 |
| chr14 | 100695294 | 100695426 |
| chr14 | 100822239 | 100822361 |
| chr14 | 100947071 | 100947221 |
| chr14 | 101196646 | 101196788 |
| chr14 | 101275888 | 101276030 |
| chr14 | 101443221 | 101443377 |
| chr14 | 101679171 | 101679295 |
| chr14 | 101726259 | 101726383 |
| chr14 | 101952320 | 101952470 |
| chr14 | 102100070 | 102100208 |
| chr14 | 102254922 | 102255043 |
| chr14 | 102429088 | 102429266 |
| chr14 | 102539723 | 102539847 |
| chr14 | 102628267 | 102628393 |
| chr14 | 102783923 | 102784059 |
| chr14 | 102986336 | 102986476 |
| chr14 | 103078025 | 103078153 |
| chr14 | 103292096 | 103292236 |
| chr14 | 103361684 | 103361808 |
| chr14 | 103520672 | 103520802 |
| chr14 | 103731739 | 103731871 |
| chr14 | 103838845 | 103838997 |
| chr14 | 103974928 | 103975052 |
| chr14 | 104238565 | 104238715 |
| chr14 | 104316432 | 104316556 |
| chr14 | 104528250 | 104528390 |
| chr14 | 104609333 | 104609457 |
| chr14 | 104789804 | 104789946 |
| chr14 | 104912830 | 104912964 |
| chr14 | 105088519 | 105088657 |
| chr14 | 105234352 | 105234492 |
| chr14 | 105302100 | 105302276 |
| chr14 | 105571812 | 105571948 |
| chr14 | 105713895 | 105714023 |
| chr14 | 105850168 | 105850304 |
| chr14 | 106000160 | 106000302 |
| chr14 | 106126883 | 106127004 |
| chr14 | 106215246 | 106215374 |
| chr14 | 106832904 | 106833028 |
| chr14 | 106950762 | 106950884 |
| chr14 | 107223148 | 107223286 |
| chr14 | 107245243 | 107245365 |
| chr15 | 20072384 | 20072512 |
| chr15 | 20114843 | 20114967 |
| chr15 | 20115555 | 20115709 |
| chr15 | 20136055 | 20136193 |
| chr15 | 20146121 | 20146241 |
| chr15 | 20150540 | 20150662 |
| chr15 | 20171496 | 20171634 |
| chr15 | 20189358 | 20189479 |
| chr15 | 20329610 | 20329766 |
| chr15 | 20337168 | 20337290 |
| chr15 | 20414191 | 20414321 |
| chr15 | 20414328 | 20414456 |
| chr15 | 20534293 | 20534449 |
| chr15 | 20587033 | 20587157 |
| chr15 | 20758891 | 20759017 |
| chr15 | 20768840 | 20768982 |
| chr15 | 20772558 | 20772718 |
| chr15 | 20850379 | 20850500 |
| chr15 | 20878703 | 20878827 |
| chr15 | 21007934 | 21008062 |
| chr15 | 21025734 | 21025876 |
| chr15 | 21070438 | 21070566 |
| chr15 | 21162802 | 21163051 |
| chr15 | 21204207 | 21204335 |
| chr15 | 21336222 | 21336378 |
| chr15 | 21343773 | 21343895 |
| chr15 | 21885252 | 21885376 |
| chr15 | 22014597 | 22014717 |
| chr15 | 22032429 | 22032571 |
| chr15 | 22081598 | 22081726 |
| chr15 | 22174029 | 22174278 |
| chr15 | 22295910 | 22296038 |
| chr15 | 22383226 | 22383364 |
| chr15 | 22426146 | 22426270 |
| chr15 | 22513469 | 22513599 |
| chr15 | 22667796 | 22667938 |
| chr15 | 22689255 | 22689381 |
| chr15 | 22710681 | 22710841 |
| chr15 | 22714417 | 22714559 |
| chr15 | 22724371 | 22724497 |
| chr15 | 22808126 | 22808318 |
| chr15 | 22922532 | 22922656 |
| chr15 | 23050945 | 23051069 |
| chr15 | 23167053 | 23167173 |
| chr15 | 23206876 | 23207016 |
| chr15 | 23258831 | 23258971 |
| chr15 | 23425949 | 23426075 |
| chr15 | 23436238 | 23436380 |
| chr15 | 23439956 | 23440116 |
| chr15 | 23591129 | 23591255 |
| chr15 | 23601037 | 23601179 |
| chr15 | 23604749 | 23604909 |
| chr15 | 23696511 | 23696632 |
| chr15 | 23860518 | 23860915 |
| chr15 | 24030987 | 24031111 |
| chr15 | 24089949 | 24090083 |
| chr15 | 24370933 | 24371095 |
| chr15 | 24424419 | 24424539 |
| chr15 | 24481077 | 24481205 |
| chr15 | 24640063 | 24640203 |
| chr15 | 24813251 | 24813389 |
| chr15 | 24823929 | 24824108 |
| chr15 | 24945736 | 24945868 |
| chr15 | 25114747 | 25114881 |
| chr15 | 25187959 | 25188445 |
| chr15 | 25355235 | 25355355 |
| chr15 | 25576825 | 25576955 |
| chr15 | 25642963 | 25643083 |
| chr15 | 25793451 | 25793577 |
| chr15 | 25917391 | 25917515 |
| chr15 | 26128822 | 26129155 |
| chr15 | 26327193 | 26327315 |
| chr15 | 26356855 | 26356981 |
| chr15 | 26586791 | 26586937 |
| chr15 | 26667317 | 26667441 |
| chr15 | 26929208 | 26929330 |
| chr15 | 27039306 | 27039430 |
| chr15 | 27234125 | 27234255 |
| chr15 | 27294381 | 27294505 |
| chr15 | 27409941 | 27410083 |
| chr15 | 27615868 | 27616000 |
| chr15 | 27825620 | 27825756 |
| chr15 | 27936705 | 27936845 |
| chr15 | 28080378 | 28080508 |
| chr15 | 28171580 | 28171712 |
| chr15 | 28307103 | 28307475 |
| chr15 | 28526174 | 28526304 |
| chr15 | 28615003 | 28615129 |
| chr15 | 28624952 | 28625094 |
| chr15 | 28628666 | 28628826 |
| chr15 | 28651814 | 28651940 |
| chr15 | 28678939 | 28679081 |
| chr15 | 28728540 | 28728682 |
| chr15 | 28750010 | 28750135 |
| chr15 | 28773100 | 28773260 |
| chr15 | 28776832 | 28776974 |
| chr15 | 28786783 | 28786909 |
| chr15 | 28984440 | 28984580 |
| chr15 | 29024021 | 29024141 |
| chr15 | 29114166 | 29114287 |
| chr15 | 29215496 | 29215618 |
| chr15 | 29450853 | 29450991 |
| chr15 | 29634446 | 29634592 |
| chr15 | 29682420 | 29682572 |
| chr15 | 29870545 | 29870683 |
| chr15 | 30005443 | 30005567 |
| chr15 | 30193378 | 30193528 |
| chr15 | 30335957 | 30336079 |
| chr15 | 30446473 | 30446597 |
| chr15 | 30583819 | 30583969 |
| chr15 | 30702752 | 30702892 |
| chr15 | 30764759 | 30764878 |
| chr15 | 30783023 | 30783159 |
| chr15 | 30783363 | 30783497 |
| chr15 | 30847774 | 30847894 |
| chr15 | 30958849 | 30958983 |
| chr15 | 31036421 | 31036543 |
| chr15 | 31263188 | 31263320 |
| chr15 | 31392055 | 31392197 |
| chr15 | 31479203 | 31479327 |
| chr15 | 31661557 | 31661683 |
| chr15 | 31830647 | 31830769 |
| chr15 | 31909067 | 31909199 |
| chr15 | 31953540 | 31953662 |
| chr15 | 32171442 | 32171570 |
| chr15 | 32324241 | 32324365 |
| chr15 | 32418359 | 32418489 |
| chr15 | 32515093 | 32515214 |
| chr15 | 32539114 | 32539264 |
| chr15 | 32676159 | 32676282 |
| chr15 | 32744122 | 32744262 |
| chr15 | 32808618 | 32808752 |
| chr15 | 32808956 | 32809092 |
| chr15 | 32827249 | 32827368 |
| chr15 | 32889249 | 32889369 |
| chr15 | 32933470 | 32933593 |
| chr15 | 33073761 | 33073891 |
| chr15 | 33151055 | 33151195 |
| chr15 | 33266886 | 33267032 |
| chr15 | 33465137 | 33465263 |
| chr15 | 33611031 | 33611155 |
| chr15 | 33818271 | 33818419 |
| chr15 | 33932936 | 33933060 |
| chr15 | 34083328 | 34083450 |
| chr15 | 34192374 | 34192496 |
| chr15 | 34402155 | 34402299 |
| chr15 | 34467659 | 34467801 |
| chr15 | 34736292 | 34736413 |
| chr15 | 34759145 | 34759305 |
| chr15 | 34908923 | 34909047 |
| chr15 | 35165300 | 35165452 |
| chr15 | 35287820 | 35287946 |
| chr15 | 35405376 | 35405502 |
| chr15 | 35544670 | 35544792 |
| chr15 | 35690966 | 35691088 |
| chr15 | 35905677 | 35905799 |
| chr15 | 35981224 | 35981370 |
| chr15 | 36101156 | 36101292 |
| chr15 | 36294744 | 36294866 |
| chr15 | 36442325 | 36442459 |
| chr15 | 36546600 | 36546730 |
| chr15 | 36747482 | 36747640 |
| chr15 | 36904721 | 36904859 |
| chr15 | 37030382 | 37030522 |
| chr15 | 37180175 | 37180303 |
| chr15 | 37395287 | 37395419 |
| chr15 | 37566671 | 37566795 |
| chr15 | 37688467 | 37688591 |
| chr15 | 37854936 | 37855082 |
| chr15 | 37991408 | 37991542 |
| chr15 | 38175710 | 38175834 |
| chr15 | 38322375 | 38322503 |
| chr15 | 38463303 | 38463431 |
| chr15 | 38587686 | 38587818 |
| chr15 | 38786026 | 38786162 |
| chr15 | 38933858 | 38933992 |
| chr15 | 39000171 | 39000295 |
| chr15 | 39179568 | 39179692 |
| chr15 | 39344223 | 39344345 |
| chr15 | 39482513 | 39482661 |
| chr15 | 39589981 | 39590111 |
| chr15 | 39694942 | 39695092 |
| chr15 | 39982108 | 39982236 |
| chr15 | 40012074 | 40012216 |
| chr15 | 40253731 | 40253855 |
| chr15 | 40292204 | 40292328 |
| chr15 | 40496044 | 40496168 |
| chr15 | 40665370 | 40665504 |
| chr15 | 40862416 | 40862546 |
| chr15 | 41008239 | 41008363 |
| chr15 | 41141900 | 41142050 |
| chr15 | 41208800 | 41208926 |
| chr15 | 41392068 | 41392192 |
| chr15 | 41586710 | 41586850 |
| chr15 | 41648889 | 41649021 |
| chr15 | 41932450 | 41932578 |
| chr15 | 42043005 | 42043143 |
| chr15 | 42194563 | 42194695 |
| chr15 | 42290619 | 42290759 |
| chr15 | 42399694 | 42399818 |
| chr15 | 42505090 | 42505232 |
| chr15 | 42528586 | 42528710 |
| chr15 | 42573009 | 42573145 |
| chr15 | 42643445 | 42643571 |
| chr15 | 42686752 | 42686890 |
| chr15 | 42715854 | 42715978 |
| chr15 | 42780780 | 42780953 |
| chr15 | 42888993 | 42889131 |
| chr15 | 43039027 | 43039149 |
| chr15 | 43286713 | 43286891 |
| chr15 | 43344971 | 43345115 |
| chr15 | 43445245 | 43445393 |
| chr15 | 43716553 | 43716693 |
| chr15 | 43770364 | 43770496 |
| chr15 | 44016331 | 44016455 |
| chr15 | 44188802 | 44188930 |
| chr15 | 44236041 | 44236163 |
| chr15 | 44465058 | 44465258 |
| chr15 | 44551117 | 44551242 |
| chr15 | 44739736 | 44739858 |
| chr15 | 44822890 | 44823142 |
| chr15 | 44988700 | 44988966 |
| chr15 | 45106109 | 45106235 |
| chr15 | 45291261 | 45291557 |
| chr15 | 45488048 | 45488176 |
| chr15 | 45652807 | 45652943 |
| chr15 | 45768863 | 45768987 |
| chr15 | 45938571 | 45938723 |
| chr15 | 46006236 | 46006372 |
| chr15 | 46241604 | 46241728 |
| chr15 | 46298368 | 46298492 |
| chr15 | 46574901 | 46575031 |
| chr15 | 46642055 | 46642189 |
| chr15 | 46768494 | 46768646 |
| chr15 | 46925303 | 46925437 |
| chr15 | 47078826 | 47078950 |
| chr15 | 47331466 | 47331600 |
| chr15 | 47427117 | 47427243 |
| chr15 | 47619053 | 47619211 |
| chr15 | 47686602 | 47686732 |
| chr15 | 47890306 | 47890430 |
| chr15 | 48082936 | 48083078 |
| chr15 | 48091668 | 48091792 |
| chr15 | 48371897 | 48372029 |
| chr15 | 48481114 | 48481242 |
| chr15 | 48618743 | 48618871 |
| chr15 | 48811978 | 48812140 |
| chr15 | 48843508 | 48843632 |
| chr15 | 49063455 | 49063591 |
| chr15 | 49230859 | 49231009 |
| chr15 | 49417494 | 49417634 |
| chr15 | 49450892 | 49451020 |
| chr15 | 49664691 | 49664815 |
| chr15 | 49756908 | 49757034 |
| chr15 | 49891032 | 49891156 |
| chr15 | 50144749 | 50144877 |
| chr15 | 50284304 | 50284428 |
| chr15 | 50469214 | 50469338 |
| chr15 | 50588683 | 50588813 |
| chr15 | 50763308 | 50763434 |
| chr15 | 50835072 | 50835196 |
| chr15 | 50950892 | 50951018 |
| chr15 | 51224838 | 51224968 |
| chr15 | 51356021 | 51356165 |
| chr15 | 51495792 | 51495918 |
| chr15 | 51568925 | 51569069 |
| chr15 | 51792882 | 51793004 |
| chr15 | 51855984 | 51856114 |
| chr15 | 52078473 | 52078597 |
| chr15 | 52279211 | 52279339 |
| chr15 | 52343018 | 52343150 |
| chr15 | 52441237 | 52441361 |
| chr15 | 52660644 | 52660768 |
| chr15 | 52784127 | 52784257 |
| chr15 | 52922182 | 52922304 |
| chr15 | 53080341 | 53080497 |
| chr15 | 53307230 | 53307372 |
| chr15 | 53370771 | 53370895 |
| chr15 | 53493278 | 53493400 |
| chr15 | 53697429 | 53697553 |
| chr15 | 53869822 | 53869946 |
| chr15 | 53972400 | 53972526 |
| chr15 | 54110820 | 54110944 |
| chr15 | 54252350 | 54252488 |
| chr15 | 54424618 | 54424756 |
| chr15 | 54619916 | 54620050 |
| chr15 | 54834158 | 54834302 |
| chr15 | 54897480 | 54897620 |
| chr15 | 55062671 | 55062821 |
| chr15 | 55208482 | 55208616 |
| chr15 | 55327687 | 55327819 |
| chr15 | 55485923 | 55486047 |
| chr15 | 55714391 | 55714517 |
| chr15 | 55832350 | 55832480 |
| chr15 | 55951342 | 55951466 |
| chr15 | 56095780 | 56095904 |
| chr15 | 56305632 | 56305758 |
| chr15 | 56439128 | 56439264 |
| chr15 | 56630612 | 56630748 |
| chr15 | 56649871 | 56649997 |
| chr15 | 56843009 | 56843133 |
| chr15 | 57065238 | 57065366 |
| chr15 | 57205843 | 57205997 |
| chr15 | 57264332 | 57264458 |
| chr15 | 57482789 | 57482911 |
| chr15 | 57658964 | 57659088 |
| chr15 | 57706680 | 57706806 |
| chr15 | 57947572 | 57947696 |
| chr15 | 58077865 | 58078007 |
| chr15 | 58240974 | 58241098 |
| chr15 | 58338916 | 58339060 |
| chr15 | 58582386 | 58582520 |
| chr15 | 58715984 | 58716114 |
| chr15 | 58792053 | 58792179 |
| chr15 | 58933921 | 58934055 |
| chr15 | 59139213 | 59139337 |
| chr15 | 59325712 | 59325842 |
| chr15 | 59427725 | 59427859 |
| chr15 | 59601372 | 59601510 |
| chr15 | 59689714 | 59689872 |
| chr15 | 59855370 | 59855492 |
| chr15 | 60044400 | 60044532 |
| chr15 | 60191490 | 60191618 |
| chr15 | 60293624 | 60293760 |
| chr15 | 60510118 | 60510258 |
| chr15 | 60598956 | 60599084 |
| chr15 | 60812239 | 60812365 |
| chr15 | 60873047 | 60873199 |
| chr15 | 61061179 | 61061309 |
| chr15 | 61146005 | 61146159 |
| chr15 | 61320812 | 61320954 |
| chr15 | 61455963 | 61456087 |
| chr15 | 61662460 | 61662606 |
| chr15 | 61777238 | 61777370 |
| chr15 | 61914694 | 61914832 |
| chr15 | 62133659 | 62133813 |
| chr15 | 62282036 | 62282192 |
| chr15 | 62458995 | 62459119 |
| chr15 | 62514544 | 62514684 |
| chr15 | 62647539 | 62647671 |
| chr15 | 62859467 | 62859609 |
| chr15 | 63078317 | 63078441 |
| chr15 | 63193979 | 63194113 |
| chr15 | 63343278 | 63343406 |
| chr15 | 63509108 | 63509236 |
| chr15 | 63627363 | 63627487 |
| chr15 | 63816364 | 63816490 |
| chr15 | 63885641 | 63885787 |
| chr15 | 64052068 | 64052210 |
| chr15 | 64188634 | 64188877 |
| chr15 | 64294084 | 64294228 |
| chr15 | 64576299 | 64576420 |
| chr15 | 64631280 | 64631468 |
| chr15 | 64851102 | 64851281 |
| chr15 | 64893787 | 64893933 |
| chr15 | 65095855 | 65096061 |
| chr15 | 65224602 | 65224726 |
| chr15 | 65481580 | 65481857 |
| chr15 | 65556396 | 65556520 |
| chr15 | 65744539 | 65744667 |
| chr15 | 65805315 | 65805437 |
| chr15 | 66012370 | 66012500 |
| chr15 | 66181846 | 66181982 |
| chr15 | 66245415 | 66245553 |
| chr15 | 66502575 | 66502697 |
| chr15 | 66570449 | 66570573 |
| chr15 | 66719193 | 66719331 |
| chr15 | 66911037 | 66911177 |
| chr15 | 67136066 | 67136204 |
| chr15 | 67239906 | 67240038 |
| chr15 | 67391805 | 67391943 |
| chr15 | 67442219 | 67442369 |
| chr15 | 67716482 | 67716620 |
| chr15 | 67773147 | 67773279 |
| chr15 | 68017690 | 68017838 |
| chr15 | 68182355 | 68182479 |
| chr15 | 68290878 | 68291024 |
| chr15 | 68391865 | 68391993 |
| chr15 | 68499263 | 68499405 |
| chr15 | 68704047 | 68704189 |
| chr15 | 68931965 | 68932091 |
| chr15 | 69027849 | 69027971 |
| chr15 | 69147056 | 69147196 |
| chr15 | 69280011 | 69280139 |
| chr15 | 69501848 | 69501976 |
| chr15 | 69600571 | 69600709 |
| chr15 | 69697443 | 69697587 |
| chr15 | 69936043 | 69936175 |
| chr15 | 70004733 | 70004857 |
| chr15 | 70226115 | 70226277 |
| chr15 | 70339170 | 70339316 |
| chr15 | 70528035 | 70528159 |
| chr15 | 70622277 | 70622407 |
| chr15 | 70819110 | 70819232 |
| chr15 | 70925672 | 70925812 |
| chr15 | 71116892 | 71117028 |
| chr15 | 71225981 | 71226107 |
| chr15 | 71406289 | 71406415 |
| chr15 | 71506073 | 71506211 |
| chr15 | 71679895 | 71680019 |
| chr15 | 71884948 | 71885072 |
| chr15 | 72082588 | 72082712 |
| chr15 | 72132554 | 72132696 |
| chr15 | 72269273 | 72269425 |
| chr15 | 72430443 | 72430583 |
| chr15 | 72686873 | 72687165 |
| chr15 | 72831308 | 72831446 |
| chr15 | 72973281 | 72973441 |
| chr15 | 73126454 | 73126578 |
| chr15 | 73203047 | 73203173 |
| chr15 | 73383201 | 73383329 |
| chr15 | 73532983 | 73533105 |
| chr15 | 73680255 | 73680387 |
| chr15 | 73836125 | 73836249 |
| chr15 | 73985169 | 73985309 |
| chr15 | 74162834 | 74162964 |
| chr15 | 74291185 | 74291311 |
| chr15 | 74339813 | 74339957 |
| chr15 | 74520054 | 74520194 |
| chr15 | 74779263 | 74779395 |
| chr15 | 74856059 | 74856197 |
| chr15 | 75052841 | 75052977 |
| chr15 | 75196086 | 75196226 |
| chr15 | 75286086 | 75286218 |
| chr15 | 75533484 | 75533605 |
| chr15 | 75639087 | 75639208 |
| chr15 | 75706206 | 75706332 |
| chr15 | 75893052 | 75893176 |
| chr15 | 76134599 | 76134735 |
| chr15 | 76188613 | 76188763 |
| chr15 | 76373461 | 76373597 |
| chr15 | 76543902 | 76544030 |
| chr15 | 76655848 | 76655972 |
| chr15 | 76800294 | 76800436 |
| chr15 | 76993057 | 76993187 |
| chr15 | 77084789 | 77084935 |
| chr15 | 77223166 | 77223328 |
| chr15 | 77436111 | 77436239 |
| chr15 | 77493088 | 77493218 |
| chr15 | 77731939 | 77732083 |
| chr15 | 77811196 | 77811320 |
| chr15 | 77972686 | 77972820 |
| chr15 | 78098008 | 78098146 |
| chr15 | 78259134 | 78259310 |
| chr15 | 78521061 | 78521211 |
| chr15 | 78622377 | 78622515 |
| chr15 | 78833574 | 78833698 |
| chr15 | 78887792 | 78887920 |
| chr15 | 79088442 | 79088563 |
| chr15 | 79184329 | 79184475 |
| chr15 | 79392474 | 79392598 |
| chr15 | 79481977 | 79482101 |
| chr15 | 79597174 | 79597296 |
| chr15 | 79742890 | 79743018 |
| chr15 | 79909651 | 79909777 |
| chr15 | 80139123 | 80139273 |
| chr15 | 80322798 | 80322942 |
| chr15 | 80411185 | 80411315 |
| chr15 | 80528329 | 80528457 |
| chr15 | 80740472 | 80740620 |
| chr15 | 80923024 | 80923146 |
| chr15 | 81011170 | 81011296 |
| chr15 | 81091793 | 81091927 |
| chr15 | 81350951 | 81351107 |
| chr15 | 81431089 | 81431215 |
| chr15 | 81547275 | 81547401 |
| chr15 | 81821718 | 81821872 |
| chr15 | 81895102 | 81895224 |
| chr15 | 82087580 | 82087708 |
| chr15 | 82255480 | 82255618 |
| chr15 | 82301520 | 82301644 |
| chr15 | 82573209 | 82573331 |
| chr15 | 82662240 | 82662386 |
| chr15 | 83038781 | 83038927 |
| chr15 | 83167507 | 83167653 |
| chr15 | 83223367 | 83223495 |
| chr15 | 83391501 | 83391623 |
| chr15 | 83551225 | 83551347 |
| chr15 | 83769793 | 83769917 |
| chr15 | 83934658 | 83934816 |
| chr15 | 84074414 | 84074544 |
| chr15 | 84206898 | 84207028 |
| chr15 | 84250602 | 84250730 |
| chr15 | 84445007 | 84445139 |
| chr15 | 84644799 | 84644925 |
| chr15 | 84728779 | 84729046 |
| chr15 | 84919741 | 84919863 |
| chr15 | 85125241 | 85125365 |
| chr15 | 85142112 | 85142351 |
| chr15 | 85329522 | 85329644 |
| chr15 | 85480614 | 85480736 |
| chr15 | 85717399 | 85717523 |
| chr15 | 85861077 | 85861205 |
| chr15 | 85936151 | 85936277 |
| chr15 | 86126239 | 86126363 |
| chr15 | 86237888 | 86238014 |
| chr15 | 86369236 | 86369382 |
| chr15 | 86570689 | 86570839 |
| chr15 | 86743134 | 86743264 |
| chr15 | 86815745 | 86815885 |
| chr15 | 86980812 | 86980934 |
| chr15 | 87141715 | 87141853 |
| chr15 | 87364769 | 87364903 |
| chr15 | 87479607 | 87479737 |
| chr15 | 87679373 | 87679503 |
| chr15 | 87766711 | 87766835 |
| chr15 | 87928452 | 87928576 |
| chr15 | 88121020 | 88121166 |
| chr15 | 88164065 | 88164199 |
| chr15 | 88329330 | 88329466 |
| chr15 | 88584180 | 88584336 |
| chr15 | 88730378 | 88730500 |
| chr15 | 88876449 | 88876571 |
| chr15 | 88995652 | 88995902 |
| chr15 | 89065089 | 89065235 |
| chr15 | 89225738 | 89225862 |
| chr15 | 89479785 | 89479923 |
| chr15 | 89549326 | 89549454 |
| chr15 | 89691690 | 89691822 |
| chr15 | 89805509 | 89805655 |
| chr15 | 90028508 | 90028658 |
| chr15 | 90114235 | 90114363 |
| chr15 | 90374725 | 90374869 |
| chr15 | 90519762 | 90519894 |
| chr15 | 90668927 | 90669067 |
| chr15 | 90788804 | 90788934 |
| chr15 | 90943419 | 90943549 |
| chr15 | 91028372 | 91028496 |
| chr15 | 91194949 | 91195075 |
| chr15 | 91400124 | 91400296 |
| chr15 | 91489647 | 91489771 |
| chr15 | 91726125 | 91726269 |
| chr15 | 91744425 | 91744549 |
| chr15 | 91909785 | 91909927 |
| chr15 | 92063613 | 92063769 |
| chr15 | 92270279 | 92270411 |
| chr15 | 92441813 | 92441963 |
| chr15 | 92634093 | 92634219 |
| chr15 | 92692868 | 92692994 |
| chr15 | 92868972 | 92869114 |
| chr15 | 93006004 | 93006156 |
| chr15 | 93193844 | 93193974 |
| chr15 | 93319537 | 93319691 |
| chr15 | 93506797 | 93506919 |
| chr15 | 93564920 | 93565058 |
| chr15 | 93800589 | 93800711 |
| chr15 | 93895167 | 93895293 |
| chr15 | 94106360 | 94106510 |
| chr15 | 94276748 | 94276890 |
| chr15 | 94355298 | 94355438 |
| chr15 | 94540713 | 94540855 |
| chr15 | 94648977 | 94649135 |
| chr15 | 94889151 | 94889273 |
| chr15 | 94988800 | 94988922 |
| chr15 | 95138646 | 95138776 |
| chr15 | 95308049 | 95308173 |
| chr15 | 95434043 | 95434197 |
| chr15 | 95525475 | 95525599 |
| chr15 | 95753196 | 95753350 |
| chr15 | 95856544 | 95856670 |
| chr15 | 95958591 | 95958725 |
| chr15 | 96209923 | 96210051 |
| chr15 | 96250772 | 96250928 |
| chr15 | 96473343 | 96473465 |
| chr15 | 96572017 | 96572151 |
| chr15 | 96835235 | 96835359 |
| chr15 | 96905975 | 96906115 |
| chr15 | 97123901 | 97124051 |
| chr15 | 97188622 | 97188752 |
| chr15 | 97388282 | 97388408 |
| chr15 | 97501953 | 97502077 |
| chr15 | 97639480 | 97639624 |
| chr15 | 97764178 | 97764306 |
| chr15 | 97939059 | 97939235 |
| chr15 | 98178624 | 98178748 |
| chr15 | 98331320 | 98331450 |
| chr15 | 98479454 | 98479620 |
| chr15 | 98583826 | 98583958 |
| chr15 | 98662946 | 98663076 |
| chr15 | 98930493 | 98930619 |
| chr15 | 99029833 | 99029957 |
| chr15 | 99095737 | 99095863 |
| chr15 | 99254492 | 99254630 |
| chr15 | 99504347 | 99504503 |
| chr15 | 99641410 | 99641546 |
| chr15 | 99831671 | 99831837 |
| chr15 | 99883582 | 99883728 |
| chr15 | 100023123 | 100023261 |
| chr15 | 100219304 | 100219434 |
| chr15 | 100411426 | 100411552 |
| chr15 | 100477056 | 100477180 |
| chr15 | 100717373 | 100717523 |
| chr15 | 100854515 | 100854643 |
| chr15 | 100912841 | 100912967 |
| chr15 | 101149308 | 101149434 |
| chr15 | 101236697 | 101236821 |
| chr15 | 101389635 | 101389759 |
| chr15 | 101632240 | 101632370 |
| chr15 | 101767555 | 101767679 |
| chr15 | 101839467 | 101839625 |
| chr15 | 101998140 | 101998278 |
| chr15 | 102229112 | 102229582 |
| chr15 | 102376711 | 102376835 |
| chr15 | 102519444 | 102519568 |
| chr16 | 221009 | 221145 |
| chr16 | 355986 | 356107 |
| chr16 | 543321 | 543445 |
| chr16 | 588002 | 588142 |
| chr16 | 748993 | 749139 |
| chr16 | 933722 | 933848 |
| chr16 | 1036546 | 1036713 |
| chr16 | 1186180 | 1186316 |
| chr16 | 1432057 | 1432307 |
| chr16 | 1593753 | 1593877 |
| chr16 | 1683356 | 1683502 |
| chr16 | 1863456 | 1863608 |
| chr16 | 1943354 | 1943492 |
| chr16 | 2145857 | 2146191 |
| chr16 | 2250555 | 2250676 |
| chr16 | 2386128 | 2386404 |
| chr16 | 2555695 | 2555816 |
| chr16 | 2725004 | 2725136 |
| chr16 | 2934116 | 2934262 |
| chr16 | 2969790 | 2969930 |
| chr16 | 3202990 | 3203116 |
| chr16 | 3274791 | 3274945 |
| chr16 | 3428024 | 3428146 |
| chr16 | 3589066 | 3589190 |
| chr16 | 3747130 | 3747254 |
| chr16 | 3873784 | 3873921 |
| chr16 | 4126447 | 4126583 |
| chr16 | 4231466 | 4231630 |
| chr16 | 4310817 | 4310949 |
| chr16 | 4462819 | 4462949 |
| chr16 | 4609712 | 4609842 |
| chr16 | 4873188 | 4873330 |
| chr16 | 4911165 | 4911287 |
| chr16 | 5194370 | 5194514 |
| chr16 | 5266725 | 5266849 |
| chr16 | 5354423 | 5354553 |
| chr16 | 5604578 | 5604712 |
| chr16 | 5774985 | 5775109 |
| chr16 | 5945224 | 5945350 |
| chr16 | 6080339 | 6080471 |
| chr16 | 6245180 | 6245304 |
| chr16 | 6285031 | 6285165 |
| chr16 | 6523408 | 6523532 |
| chr16 | 6578083 | 6578219 |
| chr16 | 6846839 | 6846963 |
| chr16 | 6917754 | 6917878 |
| chr16 | 7019763 | 7019897 |
| chr16 | 7158674 | 7158796 |
| chr16 | 7348103 | 7348233 |
| chr16 | 7479886 | 7480036 |
| chr16 | 7744534 | 7744688 |
| chr16 | 7753671 | 7753795 |
| chr16 | 8033092 | 8033216 |
| chr16 | 8125375 | 8125521 |
| chr16 | 8241807 | 8241929 |
| chr16 | 8382834 | 8382958 |
| chr16 | 8626915 | 8627039 |
| chr16 | 8692992 | 8693231 |
| chr16 | 8811353 | 8811503 |
| chr16 | 9048262 | 9048386 |
| chr16 | 9229737 | 9229859 |
| chr16 | 9382289 | 9382413 |
| chr16 | 9516592 | 9516726 |
| chr16 | 9673378 | 9673504 |
| chr16 | 9754808 | 9754960 |
| chr16 | 9897705 | 9897841 |
| chr16 | 10099491 | 10099617 |
| chr16 | 10197667 | 10197841 |
| chr16 | 10364507 | 10364641 |
| chr16 | 10482246 | 10482400 |
| chr16 | 10650508 | 10650644 |
| chr16 | 10894071 | 10894209 |
| chr16 | 11048300 | 11048438 |
| chr16 | 11195991 | 11196129 |
| chr16 | 11312309 | 11312455 |
| chr16 | 11451217 | 11451341 |
| chr16 | 11523154 | 11523296 |
| chr16 | 11690717 | 11690863 |
| chr16 | 11809639 | 11809773 |
| chr16 | 11986784 | 11986922 |
| chr16 | 12105483 | 12105609 |
| chr16 | 12314361 | 12314519 |
| chr16 | 12462728 | 12462852 |
| chr16 | 12675785 | 12675923 |
| chr16 | 12821983 | 12822115 |
| chr16 | 12910306 | 12910450 |
| chr16 | 13129165 | 13129295 |
| chr16 | 13266074 | 13266204 |
| chr16 | 13336354 | 13336504 |
| chr16 | 13525048 | 13525186 |
| chr16 | 13717796 | 13717922 |
| chr16 | 13821967 | 13822107 |
| chr16 | 13969805 | 13969943 |
| chr16 | 14144708 | 14144832 |
| chr16 | 14235490 | 14235614 |
| chr16 | 14479236 | 14479366 |
| chr16 | 14542463 | 14542586 |
| chr16 | 14775104 | 14775225 |
| chr16 | 14917950 | 14918161 |
| chr16 | 15006592 | 15006726 |
| chr16 | 15057203 | 15057324 |
| chr16 | 15168138 | 15168314 |
| chr16 | 15180677 | 15180797 |
| chr16 | 15247244 | 15247378 |
| chr16 | 15375260 | 15375396 |
| chr16 | 15492941 | 15493086 |
| chr16 | 15542109 | 15542233 |
| chr16 | 15630393 | 15630529 |
| chr16 | 15795408 | 15795560 |
| chr16 | 15927503 | 15927641 |
| chr16 | 16077784 | 16077930 |
| chr16 | 16267753 | 16267875 |
| chr16 | 16316715 | 16316926 |
| chr16 | 16405248 | 16405382 |
| chr16 | 16568387 | 16568523 |
| chr16 | 16620288 | 16620418 |
| chr16 | 16726690 | 16726878 |
| chr16 | 16855463 | 16855587 |
| chr16 | 16887012 | 16887138 |
| chr16 | 16958329 | 16958487 |
| chr16 | 17170283 | 17170427 |
| chr16 | 17214401 | 17214529 |
| chr16 | 17497603 | 17497735 |
| chr16 | 17628634 | 17628758 |
| chr16 | 17746523 | 17746647 |
| chr16 | 17846534 | 17846658 |
| chr16 | 17981740 | 17981862 |
| chr16 | 18119267 | 18119397 |
| chr16 | 18279468 | 18279598 |
| chr16 | 18331179 | 18331315 |
| chr16 | 18494478 | 18494612 |
| chr16 | 18582962 | 18583173 |
| chr16 | 18608824 | 18608948 |
| chr16 | 18821792 | 18821916 |
| chr16 | 18869323 | 18869443 |
| chr16 | 18982953 | 18983103 |
| chr16 | 19117616 | 19117740 |
| chr16 | 19161421 | 19161545 |
| chr16 | 19319513 | 19319637 |
| chr16 | 19593963 | 19594105 |
| chr16 | 19664148 | 19664274 |
| chr16 | 19769765 | 19769899 |
| chr16 | 20006655 | 20006793 |
| chr16 | 20120857 | 20120981 |
| chr16 | 20341260 | 20341400 |
| chr16 | 20370132 | 20370256 |
| chr16 | 20525641 | 20525767 |
| chr16 | 20742901 | 20743051 |
| chr16 | 20929171 | 20929295 |
| chr16 | 20957885 | 20958007 |
| chr16 | 21205646 | 21205776 |
| chr16 | 21358140 | 21358264 |
| chr16 | 21458219 | 21458344 |
| chr16 | 21458417 | 21458563 |
| chr16 | 21499900 | 21500024 |
| chr16 | 21500040 | 21500164 |
| chr16 | 21533341 | 21533467 |
| chr16 | 21687683 | 21687827 |
| chr16 | 21824295 | 21824435 |
| chr16 | 21890481 | 21890606 |
| chr16 | 21890677 | 21890823 |
| chr16 | 21931387 | 21931512 |
| chr16 | 21931533 | 21931652 |
| chr16 | 21952510 | 21952724 |
| chr16 | 22097532 | 22097795 |
| chr16 | 22206620 | 22206744 |
| chr16 | 22399536 | 22399705 |
| chr16 | 22461712 | 22461831 |
| chr16 | 22502982 | 22503128 |
| chr16 | 22503205 | 22503325 |
| chr16 | 22746941 | 22747073 |
| chr16 | 22870648 | 22870796 |
| chr16 | 23037773 | 23037921 |
| chr16 | 23177657 | 23177799 |
| chr16 | 23308788 | 23308944 |
| chr16 | 23362642 | 23362766 |
| chr16 | 23610259 | 23610554 |
| chr16 | 23731950 | 23732080 |
| chr16 | 23833083 | 23833207 |
| chr16 | 24014555 | 24014683 |
| chr16 | 24190718 | 24190870 |
| chr16 | 24274023 | 24274167 |
| chr16 | 24495111 | 24495233 |
| chr16 | 24680159 | 24680287 |
| chr16 | 24743593 | 24743717 |
| chr16 | 24968132 | 24968268 |
| chr16 | 25121268 | 25121394 |
| chr16 | 25175076 | 25175210 |
| chr16 | 25403926 | 25404052 |
| chr16 | 25550006 | 25550130 |
| chr16 | 25635661 | 25635795 |
| chr16 | 25770708 | 25770836 |
| chr16 | 25980819 | 25980943 |
| chr16 | 26132218 | 26132364 |
| chr16 | 26264220 | 26264344 |
| chr16 | 26415843 | 26415981 |
| chr16 | 26567289 | 26567411 |
| chr16 | 26709450 | 26709576 |
| chr16 | 26829463 | 26829591 |
| chr16 | 27028850 | 27028974 |
| chr16 | 27217181 | 27217315 |
| chr16 | 27342560 | 27342684 |
| chr16 | 27515314 | 27515438 |
| chr16 | 27624678 | 27624942 |
| chr16 | 27705609 | 27705732 |
| chr16 | 27936179 | 27936323 |
| chr16 | 28117055 | 28117189 |
| chr16 | 28169470 | 28169592 |
| chr16 | 28336830 | 28336952 |
| chr16 | 28559537 | 28559661 |
| chr16 | 28616575 | 28616755 |
| chr16 | 28824599 | 28824733 |
| chr16 | 29043398 | 29043522 |
| chr16 | 29059673 | 29059797 |
| chr16 | 29095260 | 29095400 |
| chr16 | 29138073 | 29138209 |
| chr16 | 29223457 | 29223579 |
| chr16 | 29266663 | 29266785 |
| chr16 | 29311345 | 29311471 |
| chr16 | 29350909 | 29351037 |
| chr16 | 29436944 | 29437285 |
| chr16 | 29538709 | 29538833 |
| chr16 | 29538907 | 29539053 |
| chr16 | 29581612 | 29581736 |
| chr16 | 29626609 | 29626735 |
| chr16 | 29656837 | 29656965 |
| chr16 | 29860825 | 29860947 |
| chr16 | 30023696 | 30023836 |
| chr16 | 30157163 | 30157411 |
| chr16 | 30278693 | 30278817 |
| chr16 | 30278892 | 30279038 |
| chr16 | 30321317 | 30321441 |
| chr16 | 30321462 | 30321581 |
| chr16 | 30432358 | 30432485 |
| chr16 | 30577402 | 30577523 |
| chr16 | 30808977 | 30809117 |
| chr16 | 30983170 | 30983320 |
| chr16 | 31033798 | 31034063 |
| chr16 | 31214840 | 31214970 |
| chr16 | 31309706 | 31309967 |
| chr16 | 31523868 | 31524014 |
| chr16 | 31736943 | 31737083 |
| chr16 | 31888639 | 31888760 |
| chr16 | 31961801 | 31961937 |
| chr16 | 32016450 | 32016582 |
| chr16 | 32059207 | 32059351 |
| chr16 | 32070746 | 32070878 |
| chr16 | 32113031 | 32113169 |
| chr16 | 32124341 | 32124461 |
| chr16 | 32126605 | 32126743 |
| chr16 | 32138813 | 32138937 |
| chr16 | 32153197 | 32153321 |
| chr16 | 32267797 | 32267927 |
| chr16 | 32330754 | 32330874 |
| chr16 | 32368994 | 32369120 |
| chr16 | 32492501 | 32492649 |
| chr16 | 32622962 | 32623089 |
| chr16 | 32684163 | 32684293 |
| chr16 | 32811185 | 32811309 |
| chr16 | 32823747 | 32823885 |
| chr16 | 32826030 | 32826150 |
| chr16 | 32837367 | 32837505 |
| chr16 | 32844489 | 32844613 |
| chr16 | 32877588 | 32877722 |
| chr16 | 32905227 | 32905363 |
| chr16 | 32960001 | 32960133 |
| chr16 | 33002432 | 33002576 |
| chr16 | 33013995 | 33014127 |
| chr16 | 33056313 | 33056451 |
| chr16 | 33067640 | 33067788 |
| chr16 | 33069905 | 33070043 |
| chr16 | 33082052 | 33082176 |
| chr16 | 33082791 | 33082911 |
| chr16 | 33208734 | 33208864 |
| chr16 | 33265274 | 33265404 |
| chr16 | 33328222 | 33328342 |
| chr16 | 33335755 | 33335877 |
| chr16 | 33366191 | 33366315 |
| chr16 | 33444491 | 33444619 |
| chr16 | 33444626 | 33444759 |
| chr16 | 33654065 | 33654197 |
| chr16 | 33665289 | 33665433 |
| chr16 | 33707607 | 33707739 |
| chr16 | 33769973 | 33770109 |
| chr16 | 33797599 | 33797733 |
| chr16 | 33967139 | 33967263 |
| chr16 | 34180089 | 34180267 |
| chr16 | 34371440 | 34371611 |
| chr16 | 34481341 | 34481537 |
| chr16 | 34699991 | 34700282 |
| chr16 | 34864272 | 34864414 |
| chr16 | 35047084 | 35047210 |
| chr16 | 35103219 | 35103359 |
| chr16 | 35147305 | 35147483 |
| chr16 | 35157983 | 35158121 |
| chr16 | 35169055 | 35169175 |
| chr16 | 46456821 | 46456941 |
| chr16 | 46474133 | 46474271 |
| chr16 | 46481490 | 46481614 |
| chr16 | 46501705 | 46501826 |
| chr16 | 46640209 | 46640330 |
| chr16 | 46844697 | 46844835 |
| chr16 | 47017997 | 47018133 |
| chr16 | 47155235 | 47155489 |
| chr16 | 47276734 | 47276878 |
| chr16 | 47487244 | 47487508 |
| chr16 | 47581608 | 47581779 |
| chr16 | 47708582 | 47708749 |
| chr16 | 47950860 | 47951108 |
| chr16 | 47982136 | 47982384 |
| chr16 | 48202008 | 48202129 |
| chr16 | 48254265 | 48254482 |
| chr16 | 48422228 | 48422494 |
| chr16 | 48623469 | 48623609 |
| chr16 | 48826711 | 48826843 |
| chr16 | 48976100 | 48976228 |
| chr16 | 49089340 | 49089464 |
| chr16 | 49245648 | 49245770 |
| chr16 | 49417204 | 49417328 |
| chr16 | 49594331 | 49594455 |
| chr16 | 49656010 | 49656148 |
| chr16 | 49809327 | 49809451 |
| chr16 | 50017417 | 50017541 |
| chr16 | 50071178 | 50071318 |
| chr16 | 50220211 | 50220355 |
| chr16 | 50413146 | 50413284 |
| chr16 | 50641654 | 50641832 |
| chr16 | 50792616 | 50792744 |
| chr16 | 50896179 | 50896317 |
| chr16 | 50979458 | 50979602 |
| chr16 | 51193383 | 51193716 |
| chr16 | 51358633 | 51358759 |
| chr16 | 51479297 | 51479425 |
| chr16 | 51626941 | 51627065 |
| chr16 | 51757186 | 51757330 |
| chr16 | 51929187 | 51929337 |
| chr16 | 52065654 | 52065804 |
| chr16 | 52224932 | 52225076 |
| chr16 | 52301772 | 52301896 |
| chr16 | 52547959 | 52548083 |
| chr16 | 52712755 | 52712883 |
| chr16 | 52815322 | 52815446 |
| chr16 | 52908377 | 52908543 |
| chr16 | 53139548 | 53139688 |
| chr16 | 53246495 | 53246641 |
| chr16 | 53429685 | 53429841 |
| chr16 | 53600044 | 53600180 |
| chr16 | 53792383 | 53792527 |
| chr16 | 53875394 | 53875548 |
| chr16 | 54042022 | 54042174 |
| chr16 | 54129866 | 54130002 |
| chr16 | 54370561 | 54370731 |
| chr16 | 54424933 | 54425059 |
| chr16 | 54590421 | 54590549 |
| chr16 | 54784241 | 54784389 |
| chr16 | 54929843 | 54929981 |
| chr16 | 55103629 | 55103781 |
| chr16 | 55211484 | 55211608 |
| chr16 | 55379555 | 55379695 |
| chr16 | 55548232 | 55548356 |
| chr16 | 55615699 | 55615845 |
| chr16 | 55778091 | 55778215 |
| chr16 | 56003890 | 56004014 |
| chr16 | 56146394 | 56146536 |
| chr16 | 56302352 | 56302476 |
| chr16 | 56434678 | 56434830 |
| chr16 | 56556712 | 56556844 |
| chr16 | 56661101 | 56661233 |
| chr16 | 56840242 | 56840384 |
| chr16 | 57034601 | 57034731 |
| chr16 | 57117369 | 57117491 |
| chr16 | 57256867 | 57257033 |
| chr16 | 57436075 | 57436211 |
| chr16 | 57682505 | 57682625 |
| chr16 | 57748793 | 57748915 |
| chr16 | 57915631 | 57915763 |
| chr16 | 58058428 | 58058552 |
| chr16 | 58170339 | 58170471 |
| chr16 | 58416007 | 58416143 |
| chr16 | 58471034 | 58471174 |
| chr16 | 58625682 | 58625804 |
| chr16 | 58768939 | 58769107 |
| chr16 | 58965610 | 58965768 |
| chr16 | 59088706 | 59088842 |
| chr16 | 59288255 | 59288413 |
| chr16 | 59446013 | 59446171 |
| chr16 | 59638333 | 59638481 |
| chr16 | 59673691 | 59673815 |
| chr16 | 59921551 | 59921677 |
| chr16 | 60039474 | 60039612 |
| chr16 | 60221561 | 60221695 |
| chr16 | 60277547 | 60277671 |
| chr16 | 60505938 | 60506076 |
| chr16 | 60575216 | 60575340 |
| chr16 | 60823055 | 60823179 |
| chr16 | 60939993 | 60940135 |
| chr16 | 61079807 | 61079929 |
| chr16 | 61284092 | 61284213 |
| chr16 | 61445086 | 61445215 |
| chr16 | 61461389 | 61461517 |
| chr16 | 61714205 | 61714337 |
| chr16 | 61859957 | 61860095 |
| chr16 | 61910642 | 61910782 |
| chr16 | 62075859 | 62075989 |
| chr16 | 62323010 | 62323146 |
| chr16 | 62454969 | 62455095 |
| chr16 | 62578059 | 62578215 |
| chr16 | 62763462 | 62763596 |
| chr16 | 62849445 | 62849569 |
| chr16 | 62970051 | 62970177 |
| chr16 | 63198013 | 63198143 |
| chr16 | 63299466 | 63299594 |
| chr16 | 63549495 | 63549617 |
| chr16 | 63591293 | 63591419 |
| chr16 | 63803977 | 63804099 |
| chr16 | 63856940 | 63857064 |
| chr16 | 64107011 | 64107135 |
| chr16 | 64232472 | 64232594 |
| chr16 | 64303693 | 64303817 |
| chr16 | 64492958 | 64493090 |
| chr16 | 64692667 | 64692813 |
| chr16 | 64844666 | 64844792 |
| chr16 | 65050310 | 65050440 |
| chr16 | 65158043 | 65158193 |
| chr16 | 65314010 | 65314156 |
| chr16 | 65440911 | 65441045 |
| chr16 | 65633204 | 65633328 |
| chr16 | 65735659 | 65735785 |
| chr16 | 65879359 | 65879483 |
| chr16 | 65969791 | 65969929 |
| chr16 | 66243681 | 66243803 |
| chr16 | 66273076 | 66273216 |
| chr16 | 66496845 | 66496975 |
| chr16 | 66673129 | 66673253 |
| chr16 | 66797774 | 66797942 |
| chr16 | 66854039 | 66854163 |
| chr16 | 67077834 | 67077984 |
| chr16 | 67276990 | 67277245 |
| chr16 | 67361468 | 67361733 |
| chr16 | 67459289 | 67459427 |
| chr16 | 67664674 | 67664817 |
| chr16 | 67775365 | 67775621 |
| chr16 | 67936790 | 67936982 |
| chr16 | 68152067 | 68152197 |
| chr16 | 68219889 | 68220029 |
| chr16 | 68486838 | 68486960 |
| chr16 | 68603718 | 68603866 |
| chr16 | 68702021 | 68702149 |
| chr16 | 68876631 | 68876763 |
| chr16 | 68982243 | 68982490 |
| chr16 | 69175948 | 69176204 |
| chr16 | 69286112 | 69286396 |
| chr16 | 69412363 | 69412484 |
| chr16 | 69688325 | 69688479 |
| chr16 | 69808936 | 69809060 |
| chr16 | 69959731 | 69959909 |
| chr16 | 70014192 | 70014316 |
| chr16 | 70195838 | 70195960 |
| chr16 | 70253819 | 70253939 |
| chr16 | 70359015 | 70359263 |
| chr16 | 70596138 | 70596278 |
| chr16 | 70742492 | 70742616 |
| chr16 | 70840452 | 70840578 |
| chr16 | 71024695 | 71024827 |
| chr16 | 71087362 | 71087488 |
| chr16 | 71279455 | 71279581 |
| chr16 | 71361802 | 71361960 |
| chr16 | 71589300 | 71589430 |
| chr16 | 71678981 | 71679105 |
| chr16 | 71877147 | 71877283 |
| chr16 | 72038309 | 72038437 |
| chr16 | 72201045 | 72201167 |
| chr16 | 72329458 | 72329580 |
| chr16 | 72495883 | 72496009 |
| chr16 | 72683207 | 72683343 |
| chr16 | 72738934 | 72739088 |
| chr16 | 72956222 | 72956356 |
| chr16 | 73137836 | 73137972 |
| chr16 | 73290250 | 73290380 |
| chr16 | 73363278 | 73363402 |
| chr16 | 73525185 | 73525311 |
| chr16 | 73644221 | 73644359 |
| chr16 | 73793480 | 73793634 |
| chr16 | 74045641 | 74045765 |
| chr16 | 74070604 | 74070740 |
| chr16 | 74258852 | 74258984 |
| chr16 | 74357615 | 74357743 |
| chr16 | 74421585 | 74421709 |
| chr16 | 74538958 | 74539081 |
| chr16 | 74791366 | 74791494 |
| chr16 | 74884032 | 74884158 |
| chr16 | 75008306 | 75008430 |
| chr16 | 75101730 | 75101876 |
| chr16 | 75388695 | 75388819 |
| chr16 | 75520952 | 75521118 |
| chr16 | 75676951 | 75677081 |
| chr16 | 75783081 | 75783205 |
| chr16 | 75974811 | 75974933 |
| chr16 | 76053972 | 76054100 |
| chr16 | 76200356 | 76200506 |
| chr16 | 76351819 | 76351957 |
| chr16 | 76566091 | 76566213 |
| chr16 | 76618932 | 76619078 |
| chr16 | 76769829 | 76769967 |
| chr16 | 77047340 | 77047464 |
| chr16 | 77128402 | 77128542 |
| chr16 | 77345721 | 77345871 |
| chr16 | 77435931 | 77436069 |
| chr16 | 77609659 | 77609791 |
| chr16 | 77713248 | 77713390 |
| chr16 | 77923446 | 77923574 |
| chr16 | 78057055 | 78057181 |
| chr16 | 78225284 | 78225428 |
| chr16 | 78331517 | 78331639 |
| chr16 | 78458430 | 78458568 |
| chr16 | 78581056 | 78581180 |
| chr16 | 78807680 | 78807830 |
| chr16 | 78905979 | 78906109 |
| chr16 | 79014687 | 79014843 |
| chr16 | 79242702 | 79242850 |
| chr16 | 79331907 | 79332049 |
| chr16 | 79582340 | 79582462 |
| chr16 | 79671688 | 79671826 |
| chr16 | 79834938 | 79835074 |
| chr16 | 79970185 | 79970319 |
| chr16 | 80075049 | 80075189 |
| chr16 | 80270252 | 80270374 |
| chr16 | 80374557 | 80374701 |
| chr16 | 80573854 | 80574004 |
| chr16 | 80691591 | 80691731 |
| chr16 | 80848561 | 80848725 |
| chr16 | 80973513 | 80973635 |
| chr16 | 81160970 | 81161116 |
| chr16 | 81395640 | 81395776 |
| chr16 | 81541097 | 81541225 |
| chr16 | 81666500 | 81666624 |
| chr16 | 81835073 | 81835197 |
| chr16 | 81992852 | 81992988 |
| chr16 | 82139203 | 82139335 |
| chr16 | 82170636 | 82170764 |
| chr16 | 82324133 | 82324261 |
| chr16 | 82582749 | 82582889 |
| chr16 | 82662383 | 82662507 |
| chr16 | 82759230 | 82759370 |
| chr16 | 83001080 | 83001224 |
| chr16 | 83100571 | 83100703 |
| chr16 | 83277636 | 83277766 |
| chr16 | 83408355 | 83408479 |
| chr16 | 83569171 | 83569299 |
| chr16 | 83683909 | 83684033 |
| chr16 | 83860695 | 83860817 |
| chr16 | 84093897 | 84094019 |
| chr16 | 84192929 | 84193077 |
| chr16 | 84268184 | 84268326 |
| chr16 | 84545194 | 84545322 |
| chr16 | 84625832 | 84625970 |
| chr16 | 84786285 | 84786429 |
| chr16 | 84947593 | 84947741 |
| chr16 | 85116917 | 85117051 |
| chr16 | 85248760 | 85248882 |
| chr16 | 85420012 | 85420150 |
| chr16 | 85537385 | 85537509 |
| chr16 | 85709534 | 85709658 |
| chr16 | 85857617 | 85857757 |
| chr16 | 85941720 | 85941846 |
| chr16 | 86082494 | 86082618 |
| chr16 | 86295314 | 86295438 |
| chr16 | 86373824 | 86373946 |
| chr16 | 86524494 | 86524628 |
| chr16 | 86678785 | 86678915 |
| chr16 | 86838381 | 86838505 |
| chr16 | 86958788 | 86958912 |
| chr16 | 87130136 | 87130270 |
| chr16 | 87303561 | 87303695 |
| chr16 | 87430187 | 87430313 |
| chr16 | 87664297 | 87664469 |
| chr16 | 87760724 | 87760874 |
| chr16 | 87976265 | 87976397 |
| chr16 | 88066378 | 88066503 |
| chr16 | 88090132 | 88090256 |
| chr16 | 88211665 | 88211787 |
| chr16 | 88229567 | 88229688 |
| chr16 | 88261715 | 88261837 |
| chr16 | 88307817 | 88307943 |
| chr16 | 88444205 | 88444330 |
| chr16 | 88520362 | 88520486 |
| chr16 | 88740044 | 88740170 |
| chr16 | 88853641 | 88853777 |
| chr16 | 88975258 | 88975386 |
| chr16 | 89194126 | 89194250 |
| chr16 | 89281041 | 89281181 |
| chr16 | 89411205 | 89411343 |
| chr16 | 89546988 | 89547128 |
| chr16 | 89734746 | 89734868 |
| chr16 | 89826183 | 89826335 |
| chr16 | 90059292 | 90059420 |
| chr16 | 90115679 | 90115805 |
| chr16 | 90190283 | 90190413 |
| chr16 | 90194416 | 90194538 |
| chr16 | 90205077 | 90205207 |
| chr17 | 3535 | 3661 |
| chr17 | 296376 | 296500 |
| chr17 | 400877 | 401011 |
| chr17 | 573250 | 573374 |
| chr17 | 656792 | 656918 |
| chr17 | 842940 | 843074 |
| chr17 | 1032167 | 1032361 |
| chr17 | 1070905 | 1071160 |
| chr17 | 1281465 | 1281599 |
| chr17 | 1429803 | 1429924 |
| chr17 | 1573271 | 1573419 |
| chr17 | 1764179 | 1764329 |
| chr17 | 1807615 | 1807757 |
| chr17 | 2027146 | 2027270 |
| chr17 | 2175122 | 2175276 |
| chr17 | 2366511 | 2366633 |
| chr17 | 2508193 | 2508319 |
| chr17 | 2562095 | 2562217 |
| chr17 | 2847633 | 2847761 |
| chr17 | 2914023 | 2914147 |
| chr17 | 3038250 | 3038374 |
| chr17 | 3208302 | 3208424 |
| chr17 | 3346751 | 3346875 |
| chr17 | 3540633 | 3540757 |
| chr17 | 3618606 | 3618730 |
| chr17 | 3813631 | 3813755 |
| chr17 | 3941158 | 3941310 |
| chr17 | 4066368 | 4066490 |
| chr17 | 4202125 | 4202247 |
| chr17 | 4431052 | 4431182 |
| chr17 | 4519097 | 4519221 |
| chr17 | 4671478 | 4671616 |
| chr17 | 4946263 | 4946411 |
| chr17 | 5006600 | 5006724 |
| chr17 | 5202819 | 5202947 |
| chr17 | 5256245 | 5256369 |
| chr17 | 5458721 | 5458865 |
| chr17 | 5644213 | 5644335 |
| chr17 | 5768839 | 5768963 |
| chr17 | 5934378 | 5934512 |
| chr17 | 6054671 | 6054825 |
| chr17 | 6192406 | 6192540 |
| chr17 | 6378940 | 6379064 |
| chr17 | 6574047 | 6574173 |
| chr17 | 6703127 | 6703271 |
| chr17 | 6850403 | 6850533 |
| chr17 | 6996763 | 6996905 |
| chr17 | 7172519 | 7172645 |
| chr17 | 7208107 | 7208259 |
| chr17 | 7396107 | 7396235 |
| chr17 | 7639661 | 7639785 |
| chr17 | 7744153 | 7744277 |
| chr17 | 7904346 | 7904472 |
| chr17 | 7971491 | 7971621 |
| chr17 | 8235218 | 8235372 |
| chr17 | 8288464 | 8288622 |
| chr17 | 8530990 | 8531116 |
| chr17 | 8682007 | 8682131 |
| chr17 | 8710429 | 8710553 |
| chr17 | 8952804 | 8952944 |
| chr17 | 9040478 | 9040618 |
| chr17 | 9207991 | 9208113 |
| chr17 | 9351990 | 9352124 |
| chr17 | 9561477 | 9561601 |
| chr17 | 9621567 | 9621705 |
| chr17 | 9759434 | 9759572 |
| chr17 | 9946708 | 9946830 |
| chr17 | 10113273 | 10113433 |
| chr17 | 10341264 | 10341388 |
| chr17 | 10459099 | 10459243 |
| chr17 | 10567429 | 10567571 |
| chr17 | 10665950 | 10666076 |
| chr17 | 10920792 | 10920914 |
| chr17 | 11037858 | 11038006 |
| chr17 | 11227294 | 11227440 |
| chr17 | 11311866 | 11312016 |
| chr17 | 11434728 | 11434852 |
| chr17 | 11639452 | 11639576 |
| chr17 | 11736050 | 11736176 |
| chr17 | 11941418 | 11941544 |
| chr17 | 12049147 | 12049289 |
| chr17 | 12197876 | 12197998 |
| chr17 | 12412547 | 12412671 |
| chr17 | 12554434 | 12554558 |
| chr17 | 12732853 | 12732993 |
| chr17 | 12794862 | 12795004 |
| chr17 | 12984568 | 12984692 |
| chr17 | 13138958 | 13139084 |
| chr17 | 13248313 | 13248439 |
| chr17 | 13455871 | 13455993 |
| chr17 | 13615258 | 13615380 |
| chr17 | 13728619 | 13728763 |
| chr17 | 13868091 | 13868241 |
| chr17 | 13971418 | 13971564 |
| chr17 | 14133516 | 14133640 |
| chr17 | 14343889 | 14344015 |
| chr17 | 14492312 | 14492444 |
| chr17 | 14646877 | 14646999 |
| chr17 | 14780118 | 14780242 |
| chr17 | 14939853 | 14939975 |
| chr17 | 15065290 | 15065416 |
| chr17 | 15287054 | 15287196 |
| chr17 | 15394292 | 15394432 |
| chr17 | 15532628 | 15532756 |
| chr17 | 15552236 | 15552414 |
| chr17 | 15637981 | 15638127 |
| chr17 | 15685197 | 15685321 |
| chr17 | 15873191 | 15873315 |
| chr17 | 15915022 | 15915146 |
| chr17 | 16136857 | 16137109 |
| chr17 | 16282148 | 16282294 |
| chr17 | 16450152 | 16450423 |
| chr17 | 16523361 | 16523485 |
| chr17 | 16792515 | 16792637 |
| chr17 | 16939529 | 16939675 |
| chr17 | 17001231 | 17001373 |
| chr17 | 17120774 | 17120898 |
| chr17 | 17379479 | 17379605 |
| chr17 | 17493939 | 17494063 |
| chr17 | 17680248 | 17680421 |
| chr17 | 17825940 | 17826080 |
| chr17 | 17932740 | 17932866 |
| chr17 | 18109186 | 18109310 |
| chr17 | 18278673 | 18278795 |
| chr17 | 18320340 | 18320460 |
| chr17 | 18321433 | 18321557 |
| chr17 | 18322004 | 18322165 |
| chr17 | 18355766 | 18355885 |
| chr17 | 18356332 | 18356456 |
| chr17 | 18357428 | 18357548 |
| chr17 | 18545201 | 18545347 |
| chr17 | 18637945 | 18638073 |
| chr17 | 18678630 | 18678751 |
| chr17 | 18754065 | 18754186 |
| chr17 | 18800034 | 18800164 |
| chr17 | 18880575 | 18880701 |
| chr17 | 18927217 | 18927338 |
| chr17 | 19146026 | 19146245 |
| chr17 | 19322016 | 19322144 |
| chr17 | 19366531 | 19366659 |
| chr17 | 19533539 | 19533669 |
| chr17 | 19761089 | 19761338 |
| chr17 | 19845711 | 19845857 |
| chr17 | 20038219 | 20038343 |
| chr17 | 20219348 | 20219472 |
| chr17 | 20393476 | 20393638 |
| chr17 | 20394569 | 20394693 |
| chr17 | 20395140 | 20395259 |
| chr17 | 20428871 | 20429031 |
| chr17 | 20429478 | 20429602 |
| chr17 | 20430575 | 20430695 |
| chr17 | 20522599 | 20522726 |
| chr17 | 20612611 | 20612730 |
| chr17 | 20613658 | 20613782 |
| chr17 | 20614274 | 20614394 |
| chr17 | 20767928 | 20768072 |
| chr17 | 20810595 | 20810770 |
| chr17 | 20871113 | 20871247 |
| chr17 | 21030979 | 21031103 |
| chr17 | 21235312 | 21235436 |
| chr17 | 21436491 | 21436669 |
| chr17 | 21519622 | 21519768 |
| chr17 | 21689128 | 21689266 |
| chr17 | 21836480 | 21836604 |
| chr17 | 22031615 | 22031765 |
| chr17 | 22104986 | 22105110 |
| chr17 | 22204551 | 22204732 |
| chr17 | 25269520 | 25269643 |
| chr17 | 25299717 | 25299842 |
| chr17 | 25390279 | 25390471 |
| chr17 | 25546615 | 25546736 |
| chr17 | 25681238 | 25681360 |
| chr17 | 25882947 | 25883099 |
| chr17 | 26052419 | 26052543 |
| chr17 | 26109485 | 26109627 |
| chr17 | 26267707 | 26267851 |
| chr17 | 26473614 | 26473752 |
| chr17 | 26688585 | 26688721 |
| chr17 | 26753441 | 26753689 |
| chr17 | 26992323 | 26992455 |
| chr17 | 27135239 | 27135484 |
| chr17 | 27260604 | 27260742 |
| chr17 | 27346474 | 27346596 |
| chr17 | 27555754 | 27555884 |
| chr17 | 27655803 | 27655927 |
| chr17 | 27876765 | 27876891 |
| chr17 | 27978376 | 27978534 |
| chr17 | 28051054 | 28051200 |
| chr17 | 28263894 | 28264048 |
| chr17 | 28488166 | 28488298 |
| chr17 | 28579284 | 28579408 |
| chr17 | 28699493 | 28699617 |
| chr17 | 28874262 | 28874383 |
| chr17 | 29086511 | 29086643 |
| chr17 | 29107486 | 29107628 |
| chr17 | 29392697 | 29392818 |
| chr17 | 29449188 | 29449383 |
| chr17 | 29637677 | 29637799 |
| chr17 | 29835322 | 29835450 |
| chr17 | 29871041 | 29871169 |
| chr17 | 30040767 | 30040889 |
| chr17 | 30187065 | 30187191 |
| chr17 | 30324130 | 30324390 |
| chr17 | 30468476 | 30468604 |
| chr17 | 30723683 | 30723804 |
| chr17 | 30835795 | 30835919 |
| chr17 | 30992368 | 30992492 |
| chr17 | 31132777 | 31132901 |
| chr17 | 31209798 | 31209932 |
| chr17 | 31404592 | 31404750 |
| chr17 | 31596157 | 31596315 |
| chr17 | 31782596 | 31782732 |
| chr17 | 31820349 | 31820471 |
| chr17 | 32055541 | 32055691 |
| chr17 | 32185291 | 32185419 |
| chr17 | 32269750 | 32269892 |
| chr17 | 32509200 | 32509328 |
| chr17 | 32574360 | 32574482 |
| chr17 | 32760663 | 32760823 |
| chr17 | 32888679 | 32888847 |
| chr17 | 33075120 | 33075244 |
| chr17 | 33181789 | 33181913 |
| chr17 | 33382723 | 33382851 |
| chr17 | 33564319 | 33564461 |
| chr17 | 33670683 | 33670819 |
| chr17 | 33857844 | 33858002 |
| chr17 | 33970895 | 33971021 |
| chr17 | 34143567 | 34143699 |
| chr17 | 34273908 | 34274042 |
| chr17 | 34392806 | 34392934 |
| chr17 | 34504613 | 34504735 |
| chr17 | 34526781 | 34526905 |
| chr17 | 34592657 | 34592779 |
| chr17 | 34628352 | 34628476 |
| chr17 | 34757668 | 34757790 |
| chr17 | 34808721 | 34808843 |
| chr17 | 34815987 | 34816108 |
| chr17 | 34965923 | 34966047 |
| chr17 | 35109895 | 35110043 |
| chr17 | 35348493 | 35348621 |
| chr17 | 35489799 | 35489921 |
| chr17 | 35635451 | 35635585 |
| chr17 | 35824091 | 35824249 |
| chr17 | 35975094 | 35975230 |
| chr17 | 36067792 | 36067916 |
| chr17 | 36178251 | 36178397 |
| chr17 | 36215228 | 36215348 |
| chr17 | 36283219 | 36283341 |
| chr17 | 36349284 | 36349406 |
| chr1: | 36410377 | 36410501 |
| chr17 | 36481844 | 36482021 |
| chr17 | 36657042 | 36657174 |
| chr17 | 36746035 | 36746159 |
| chr17 | 36890725 | 36890985 |
| chr17 | 37004933 | 37005073 |
| chr17 | 37125870 | 37126006 |
| chr17 | 37219249 | 37219373 |
| chr17 | 37443984 | 37444106 |
| chr17 | 37543250 | 37543372 |
| chr17 | 37660379 | 37660503 |
| chr17 | 37843516 | 37843638 |
| chr17 | 37990345 | 37990466 |
| chr17 | 38138923 | 38139079 |
| chr17 | 38398065 | 38398317 |
| chr17 | 38536943 | 38537071 |
| chr17 | 38582686 | 38582824 |
| chr17 | 38837687 | 38837809 |
| chr17 | 38943532 | 38943668 |
| chr17 | 39089478 | 39089636 |
| chr17 | 39185708 | 39185854 |
| chr17 | 39386311 | 39386437 |
| chr17 | 39474776 | 39474910 |
| chr17 | 39721273 | 39721413 |
| chr17 | 39796479 | 39796601 |
| chr17 | 39920205 | 39920335 |
| chr17 | 40193627 | 40193757 |
| chr17 | 40447313 | 40447451 |
| chr17 | 40519873 | 40520005 |
| chr17 | 40690030 | 40690168 |
| chr17 | 40895995 | 40896125 |
| chr17 | 41041527 | 41041787 |
| chr17 | 41127396 | 41127522 |
| chr17 | 41341922 | 41342058 |
| chr17 | 41510798 | 41510920 |
| chr17 | 41673944 | 41674076 |
| chr17 | 41845998 | 41846121 |
| chr17 | 41903982 | 41904148 |
| chr17 | 42109756 | 42109914 |
| chr17 | 42213257 | 42213405 |
| chr17 | 42443696 | 42443840 |
| chr17 | 42483943 | 42484089 |
| chr17 | 42650304 | 42650426 |
| chr17 | 42793634 | 42793770 |
| chr17 | 42942989 | 42943113 |
| chr17 | 43133539 | 43133661 |
| chr17 | 43274481 | 43274603 |
| chr17 | 43497305 | 43497445 |
| chr17 | 43512777 | 43512898 |
| chr17 | 43640853 | 43640985 |
| chr17 | 43708817 | 43708938 |
| chr17 | 43802468 | 43802590 |
| chr17 | 44037214 | 44037337 |
| chr17 | 44162639 | 44162771 |
| chr17 | 44346784 | 44346934 |
| chr17 | 44576771 | 44576903 |
| chr17 | 44800910 | 44801034 |
| chr17 | 44930273 | 44930403 |
| chr17 | 45056890 | 45057016 |
| chr17 | 45181443 | 45181579 |
| chr17 | 45337301 | 45337425 |
| chr17 | 45479873 | 45480005 |
| chr17 | 45611309 | 45611436 |
| chr17 | 45674611 | 45674735 |
| chr17 | 45865903 | 45866029 |
| chr17 | 45985332 | 45985470 |
| chr17 | 46130198 | 46130324 |
| chr17 | 46238224 | 46238346 |
| chr17 | 46372236 | 46372360 |
| chr17 | 46548994 | 46549120 |
| chr17 | 46675935 | 46676059 |
| chr17 | 46909236 | 46909421 |
| chr17 | 46978317 | 46978439 |
| chr17 | 47101933 | 47102059 |
| chr17 | 47340063 | 47340187 |
| chr17 | 47509706 | 47509834 |
| chr17 | 47611376 | 47611510 |
| chr17 | 47843768 | 47843904 |
| chr17 | 47900119 | 47900391 |
| chr17 | 48005844 | 48005968 |
| chr17 | 48178625 | 48178763 |
| chr17 | 48425877 | 48426009 |
| chr17 | 48510050 | 48510176 |
| chr17 | 48643663 | 48643785 |
| chr17 | 48770427 | 48770551 |
| chr17 | 48915797 | 48915929 |
| chr17 | 49107443 | 49107583 |
| chr17 | 49315312 | 49315456 |
| chr17 | 49459441 | 49459565 |
| chr17 | 49635672 | 49635816 |
| chr17 | 49695620 | 49695762 |
| chr17 | 49901878 | 49902036 |
| chr17 | 50038189 | 50038315 |
| chr17 | 50238377 | 50238501 |
| chr17 | 50316047 | 50316173 |
| chr17 | 50450490 | 50450626 |
| chr17 | 50577513 | 50577677 |
| chr17 | 50771242 | 50771378 |
| chr17 | 50951521 | 50951667 |
| chr17 | 51058175 | 51058305 |
| chr17 | 51168260 | 51168394 |
| chr17 | 51446631 | 51446765 |
| chr17 | 51528900 | 51529022 |
| chr17 | 51651165 | 51651307 |
| chr17 | 51751015 | 51751153 |
| chr17 | 51926866 | 51926994 |
| chr17 | 52078494 | 52078632 |
| chr17 | 52322113 | 52322237 |
| chr17 | 52442551 | 52442695 |
| chr17 | 52567729 | 52567871 |
| chr17 | 52652334 | 52652492 |
| chr17 | 52834213 | 52834381 |
| chr17 | 53012891 | 53013015 |
| chr17 | 53194733 | 53194857 |
| chr17 | 53314473 | 53314597 |
| chr17 | 53469273 | 53469395 |
| chr17 | 53576699 | 53576869 |
| chr17 | 53810749 | 53810897 |
| chr17 | 53944609 | 53944767 |
| chr17 | 54124433 | 54124569 |
| chr17 | 54291085 | 54291239 |
| chr17 | 54449503 | 54449745 |
| chr17 | 54532746 | 54532872 |
| chr17 | 54736215 | 54736339 |
| chr17 | 54889837 | 54889967 |
| chr17 | 54932761 | 54932905 |
| chr17 | 55165552 | 55165696 |
| chr17 | 55303983 | 55304123 |
| chr17 | 55379286 | 55379410 |
| chr17 | 55617223 | 55617347 |
| chr17 | 55654237 | 55654359 |
| chr17 | 55803918 | 55804054 |
| chr17 | 56044566 | 56044828 |
| chr17 | 56200758 | 56200882 |
| chr17 | 56373033 | 56373157 |
| chr17 | 56462723 | 56462847 |
| chr17 | 56687484 | 56687620 |
| chr17 | 56767952 | 56768076 |
| chr17 | 56953095 | 56953217 |
| chr17 | 57023254 | 57023378 |
| chr17 | 57165009 | 57165153 |
| chr17 | 57373038 | 57373168 |
| chr17 | 57518906 | 57519050 |
| chr17 | 57740013 | 57740161 |
| chr17 | 57800882 | 57801028 |
| chr17 | 57930594 | 57930728 |
| chr17 | 58115303 | 58115424 |
| chr17 | 58209857 | 58209978 |
| chr17 | 58361186 | 58361326 |
| chr17 | 58553183 | 58553325 |
| chr17 | 58688061 | 58688306 |
| chr17 | 58927415 | 58927539 |
| chr17 | 58990945 | 58991073 |
| chr17 | 59205492 | 59205733 |
| chr17 | 59254819 | 59254961 |
| chr17 | 59506591 | 59506731 |
| chr17 | 59627384 | 59627516 |
| chr17 | 59762398 | 59762524 |
| chr17 | 59877908 | 59878034 |
| chr17 | 60095239 | 60095367 |
| chr17 | 60289488 | 60289609 |
| chr17 | 60377387 | 60377509 |
| chr17 | 60482525 | 60482681 |
| chr17 | 60559080 | 60559202 |
| chr17 | 60643980 | 60644101 |
| chr17 | 60893423 | 60893571 |
| chr17 | 60960247 | 60960385 |
| chr17 | 61108614 | 61108737 |
| chr17 | 61217845 | 61217987 |
| chr17 | 61398745 | 61398867 |
| chr17 | 61624340 | 61624587 |
| chr17 | 61735566 | 61735823 |
| chr17 | 61873241 | 61873365 |
| chr17 | 62072581 | 62072723 |
| chr17 | 62185851 | 62185973 |
| chr17 | 62270246 | 62270378 |
| chr17 | 62475909 | 62476054 |
| chr17 | 62645375 | 62645623 |
| chr17 | 62752200 | 62752321 |
| chr17 | 62906555 | 62906687 |
| chr17 | 63117739 | 63117865 |
| chr17 | 63261067 | 63261199 |
| chr17 | 63427190 | 63427324 |
| chr17 | 63532121 | 63532249 |
| chr17 | 63702076 | 63702206 |
| chr17 | 63781527 | 63781663 |
| chr17 | 63910667 | 63910817 |
| chr17 | 64164262 | 64164402 |
| chr17 | 64315481 | 64315617 |
| chr17 | 64493817 | 64493955 |
| chr17 | 64529586 | 64529714 |
| chr17 | 64747258 | 64747384 |
| chr17 | 64857408 | 64857546 |
| chr17 | 65085524 | 65085656 |
| chr17 | 65182397 | 65182523 |
| chr17 | 65370734 | 65370856 |
| chr17 | 65440396 | 65440520 |
| chr17 | 65594384 | 65594508 |
| chr17 | 65842112 | 65842238 |
| chr17 | 65938603 | 65938725 |
| chr17 | 66080416 | 66080684 |
| chr17 | 66176875 | 66177004 |
| chr17 | 66442046 | 66442218 |
| chr17 | 66477978 | 66478114 |
| chr17 | 66626315 | 66626443 |
| chr17 | 66862721 | 66862847 |
| chr17 | 66929754 | 66929884 |
| chr17 | 67103029 | 67103157 |
| chr17 | 67285583 | 67285705 |
| chr17 | 67357517 | 67357651 |
| chr17 | 67540305 | 67540437 |
| chr17 | 67653598 | 67653722 |
| chr17 | 67941421 | 67941567 |
| chr17 | 68061707 | 68061833 |
| chr17 | 68216052 | 68216178 |
| chr17 | 68362012 | 68362142 |
| chr17 | 68434136 | 68434288 |
| chr17 | 68578884 | 68579012 |
| chr17 | 68849647 | 68849803 |
| chr17 | 68995006 | 68995158 |
| chr17 | 69064494 | 69064620 |
| chr17 | 69296954 | 69297098 |
| chr17 | 69357419 | 69357555 |
| chr17 | 69522749 | 69522873 |
| chr17 | 69712207 | 69712345 |
| chr17 | 69863631 | 69863767 |
| chr17 | 69919414 | 69919540 |
| chr17 | 70067922 | 70068056 |
| chr17 | 70275749 | 70275875 |
| chr17 | 70360238 | 70360362 |
| chr17 | 70624194 | 70624318 |
| chr17 | 70708415 | 70708549 |
| chr17 | 70858162 | 70858310 |
| chr17 | 71054277 | 71054399 |
| chr17 | 71115620 | 71115744 |
| chr17 | 71372598 | 71372744 |
| chr17 | 71450340 | 71450474 |
| chr17 | 71688095 | 71688253 |
| chr17 | 71774570 | 71774696 |
| chr17 | 71961083 | 71961223 |
| chr17 | 72014371 | 72014497 |
| chr17 | 72166949 | 72167073 |
| chr17 | 72315158 | 72315298 |
| chr17 | 72571718 | 72571858 |
| chr17 | 72641101 | 72641239 |
| chr17 | 72871057 | 72871181 |
| chr17 | 73016196 | 73016344 |
| chr17 | 73080342 | 73080490 |
| chr17 | 73326881 | 73327045 |
| chr17 | 73416756 | 73416894 |
| chr17 | 73650407 | 73650529 |
| chr17 | 73790934 | 73791060 |
| chr17 | 73850755 | 73850907 |
| chr17 | 74045136 | 74045276 |
| chr17 | 74234598 | 74234728 |
| chr17 | 74309434 | 74309562 |
| chr17 | 74541845 | 74541967 |
| chr17 | 74627568 | 74627700 |
| chr17 | 74815524 | 74815658 |
| chr17 | 74862269 | 74862409 |
| chr17 | 75144562 | 75144692 |
| chr17 | 75278435 | 75278609 |
| chr17 | 75420119 | 75420247 |
| chr17 | 75556166 | 75556298 |
| chr17 | 75727607 | 75727763 |
| chr17 | 75862631 | 75862753 |
| chr17 | 75940676 | 75940808 |
| chr17 | 76144049 | 76144173 |
| chr17 | 76302314 | 76302436 |
| chr17 | 76438953 | 76439091 |
| chr17 | 76633497 | 76633637 |
| chr17 | 76718798 | 76718922 |
| chr17 | 76931000 | 76931130 |
| chr17 | 77002701 | 77002849 |
| chr17 | 77186955 | 77187079 |
| chr17 | 77288359 | 77288483 |
| chr17 | 77545810 | 77545992 |
| chr17 | 77659389 | 77659515 |
| chr17 | 77791458 | 77791584 |
| chr17 | 77931453 | 77931579 |
| chr17 | 78007533 | 78007677 |
| chr17 | 78211644 | 78211770 |
| chr17 | 78392351 | 78392479 |
| chr17 | 78567692 | 78567826 |
| chr17 | 78725712 | 78725844 |
| chr17 | 78768507 | 78768633 |
| chr17 | 78910437 | 78910587 |
| chr17 | 79158361 | 79158485 |
| chr17 | 79323553 | 79323691 |
| chr17 | 79441011 | 79441133 |
| chr17 | 79551010 | 79551134 |
| chr17 | 79698068 | 79698189 |
| chr17 | 79934150 | 79934282 |
| chr17 | 80078200 | 80078322 |
| chr17 | 80229236 | 80229502 |
| chr17 | 80257885 | 80258129 |
| chr17 | 80415941 | 80416069 |
| chr17 | 80636326 | 80636466 |
| chr17 | 80836652 | 80836782 |
| chr17 | 80981417 | 80981542 |
| chr17 | 81019163 | 81019301 |
| chr17 | 81151007 | 81151264 |
| chr18 | 159199 | 159321 |
| chr18 | 294441 | 294571 |
| chr18 | 452022 | 452188 |
| chr18 | 585372 | 585496 |
| chr18 | 631268 | 631410 |
| chr18 | 841233 | 841383 |
| chr18 | 1047264 | 1047398 |
| chr18 | 1189533 | 1189657 |
| chr18 | 1322479 | 1322605 |
| chr18 | 1459967 | 1460115 |
| chr18 | 1513443 | 1513573 |
| chr18 | 1699384 | 1699514 |
| chr18 | 1951041 | 1951181 |
| chr18 | 2005995 | 2006117 |
| chr18 | 2189026 | 2189150 |
| chr18 | 2375728 | 2375866 |
| chr18 | 2454468 | 2454602 |
| chr18 | 2691352 | 2691474 |
| chr18 | 2815422 | 2815574 |
| chr18 | 3002868 | 3002998 |
| chr18 | 3141371 | 3141509 |
| chr18 | 3178187 | 3178331 |
| chr18 | 3439094 | 3439244 |
| chr18 | 3503436 | 3503562 |
| chr18 | 3623335 | 3623469 |
| chr18 | 3853523 | 3853659 |
| chr18 | 3998377 | 3998517 |
| chr18 | 4091947 | 4092073 |
| chr18 | 4275324 | 4275454 |
| chr18 | 4405179 | 4405305 |
| chr18 | 4596401 | 4596523 |
| chr18 | 4721600 | 4721724 |
| chr18 | 4925899 | 4926037 |
| chr18 | 4970440 | 4970580 |
| chr18 | 5155974 | 5156124 |
| chr18 | 5386594 | 5386734 |
| chr18 | 5414988 | 5415162 |
| chr18 | 5570166 | 5570288 |
| chr18 | 5841901 | 5842031 |
| chr18 | 5954306 | 5954430 |
| chr18 | 6043945 | 6044069 |
| chr18 | 6194162 | 6194291 |
| chr18 | 6357052 | 6357186 |
| chr18 | 6527097 | 6527221 |
| chr18 | 6644304 | 6644434 |
| chr18 | 6776700 | 6776872 |
| chr18 | 6913897 | 6914049 |
| chr18 | 7158418 | 7158568 |
| chr18 | 7278722 | 7278848 |
| chr18 | 7389138 | 7389296 |
| chr18 | 7564613 | 7564735 |
| chr18 | 7745126 | 7745262 |
| chr18 | 7846440 | 7846562 |
| chr18 | 8096026 | 8096148 |
| chr18 | 8217603 | 8217725 |
| chr18 | 8287769 | 8287893 |
| chr18 | 8459062 | 8459190 |
| chr18 | 8599711 | 8599837 |
| chr18 | 8734929 | 8735065 |
| chr18 | 8899552 | 8899676 |
| chr18 | 9158693 | 9158829 |
| chr18 | 9299210 | 9299334 |
| chr18 | 9366415 | 9366555 |
| chr18 | 9553702 | 9553840 |
| chr18 | 9628801 | 9628941 |
| chr18 | 9869966 | 9870100 |
| chr18 | 9929240 | 9929362 |
| chr18 | 10152770 | 10152906 |
| chr18 | 10217637 | 10217775 |
| chr18 | 10374996 | 10375122 |
| chr18 | 10551696 | 10551824 |
| chr18 | 10711730 | 10711852 |
| chr18 | 10882061 | 10882205 |
| chr18 | 11089516 | 11089686 |
| chr18 | 11249946 | 11250070 |
| chr18 | 11362541 | 11362675 |
| chr18 | 11466245 | 11466385 |
| chr18 | 11595986 | 11596138 |
| chr18 | 11788065 | 11788221 |
| chr18 | 11962818 | 11962972 |
| chr18 | 12034174 | 12034308 |
| chr18 | 12233075 | 12233196 |
| chr18 | 12357220 | 12357378 |
| chr18 | 12462019 | 12462145 |
| chr18 | 12751769 | 12751909 |
| chr18 | 12848283 | 12848435 |
| chr18 | 12942150 | 12942276 |
| chr18 | 13120311 | 13120471 |
| chr18 | 13275330 | 13275452 |
| chr18 | 13465202 | 13465326 |
| chr18 | 13540651 | 13540801 |
| chr18 | 13790787 | 13790911 |
| chr18 | 13926344 | 13926474 |
| chr18 | 14098890 | 14099014 |
| chr18 | 14183352 | 14183474 |
| chr18 | 14353742 | 14353878 |
| chr18 | 14477064 | 14477192 |
| chr18 | 14541868 | 14541996 |
| chr18 | 14707114 | 14707242 |
| chr18 | 14731081 | 14731355 |
| chr18 | 14968944 | 14969104 |
| chr18 | 15100317 | 15100463 |
| chr18 | 15249659 | 15249781 |
| chr18 | 15269277 | 15269440 |
| chr18 | 15343598 | 15343725 |
| chr18 | 18541534 | 18541657 |
| chr18 | 18552371 | 18552497 |
| chr18 | 18594225 | 18594363 |
| chr18 | 18722060 | 18722344 |
| chr18 | 18888698 | 18888969 |
| chr18 | 18965865 | 18965989 |
| chr18 | 19136465 | 19136643 |
| chr18 | 19214611 | 19214862 |
| chr18 | 19410552 | 19410748 |
| chr18 | 19512594 | 19512724 |
| chr18 | 19721285 | 19721419 |
| chr18 | 19826688 | 19826818 |
| chr18 | 20010165 | 20010293 |
| chr18 | 20212517 | 20212645 |
| chr18 | 20281304 | 20281426 |
| chr18 | 20482682 | 20482824 |
| chr18 | 20590765 | 20590901 |
| chr18 | 20830951 | 20831081 |
| chr18 | 20921232 | 20921358 |
| chr18 | 21101886 | 21102032 |
| chr18 | 21208306 | 21208438 |
| chr18 | 21360688 | 21360828 |
| chr18 | 21501974 | 21502098 |
| chr18 | 21631158 | 21631294 |
| chr18 | 21809397 | 21809521 |
| chr18 | 21929975 | 21930101 |
| chr18 | 22119791 | 22119917 |
| chr18 | 22229592 | 22229722 |
| chr18 | 22417232 | 22417384 |
| chr18 | 22541628 | 22541758 |
| chr18 | 22717086 | 22717214 |
| chr18 | 22811902 | 22812030 |
| chr18 | 22987239 | 22987371 |
| chr18 | 23256623 | 23256791 |
| chr18 | 23336297 | 23336421 |
| chr18 | 23437532 | 23437656 |
| chr18 | 23574814 | 23574938 |
| chr18 | 23755100 | 23755246 |
| chr18 | 23874770 | 23874900 |
| chr18 | 24147287 | 24147411 |
| chr18 | 24207738 | 24207884 |
| chr18 | 24427251 | 24427373 |
| chr18 | 24595782 | 24595932 |
| chr18 | 24687260 | 24687396 |
| chr18 | 24857371 | 24857499 |
| chr18 | 24977485 | 24977609 |
| chr18 | 25131339 | 25131475 |
| chr18 | 25257086 | 25257228 |
| chr18 | 25377451 | 25377599 |
| chr18 | 25629247 | 25629369 |
| chr18 | 25681996 | 25682138 |
| chr18 | 25871943 | 25872065 |
| chr18 | 26010861 | 26011009 |
| chr18 | 26186710 | 26186852 |
| chr18 | 26403299 | 26403421 |
| chr18 | 26461771 | 26461897 |
| chr18 | 26686942 | 26687066 |
| chr18 | 26816421 | 26816575 |
| chr18 | 26958355 | 26958491 |
| chr18 | 27073421 | 27073571 |
| chr18 | 27307737 | 27307877 |
| chr18 | 27407530 | 27407654 |
| chr18 | 27536688 | 27536816 |
| chr18 | 27670229 | 27670357 |
| chr18 | 27819331 | 27819471 |
| chr18 | 27951620 | 27951746 |
| chr18 | 28209568 | 28209698 |
| chr18 | 28294251 | 28294407 |
| chr18 | 28398463 | 28398603 |
| chr18 | 28513699 | 28513847 |
| chr18 | 28754554 | 28754678 |
| chr18 | 28841185 | 28841307 |
| chr18 | 29048459 | 29048591 |
| chr18 | 29156909 | 29157057 |
| chr18 | 29274307 | 29274433 |
| chr18 | 29446129 | 29446251 |
| chr18 | 29631969 | 29632117 |
| chr18 | 29853315 | 29853445 |
| chr18 | 29922885 | 29923011 |
| chr18 | 30030696 | 30030820 |
| chr18 | 30221205 | 30221349 |
| chr18 | 30399726 | 30399868 |
| chr18 | 30577327 | 30577450 |
| chr18 | 30686356 | 30686615 |
| chr18 | 30894210 | 30894334 |
| chr18 | 30943935 | 30944057 |
| chr18 | 31173474 | 31173610 |
| chr18 | 31310683 | 31310809 |
| chr18 | 31398909 | 31399037 |
| chr18 | 31659140 | 31659290 |
| chr18 | 31765654 | 31765792 |
| chr18 | 31903534 | 31903656 |
| chr18 | 32026186 | 32026320 |
| chr18 | 32112772 | 32113016 |
| chr18 | 32388330 | 32388502 |
| chr18 | 32415661 | 32415913 |
| chr18 | 32656845 | 32656995 |
| chr18 | 32718888 | 32719012 |
| chr18 | 32998578 | 32998720 |
| chr18 | 33152543 | 33152687 |
| chr18 | 33177459 | 33177581 |
| chr18 | 33432966 | 33433090 |
| chr18 | 33585658 | 33585798 |
| chr18 | 33736094 | 33736216 |
| chr18 | 33797911 | 33798039 |
| chr18 | 34049026 | 34049150 |
| chr18 | 34098218 | 34098354 |
| chr18 | 34255687 | 34255817 |
| chr18 | 34377966 | 34378094 |
| chr18 | 34576832 | 34576956 |
| chr18 | 34802362 | 34802492 |
| chr18 | 34836200 | 34836342 |
| chr18 | 35062073 | 35062223 |
| chr18 | 35152473 | 35152613 |
| chr18 | 35406200 | 35406348 |
| chr18 | 35432258 | 35432430 |
| chr18 | 35567922 | 35568068 |
| chr18 | 35758101 | 35758253 |
| chr18 | 35967944 | 35968088 |
| chr18 | 36131381 | 36131507 |
| chr18 | 36247817 | 36247949 |
| chr18 | 36383358 | 36383488 |
| chr18 | 36588660 | 36588784 |
| chr18 | 36757058 | 36757196 |
| chr18 | 36820009 | 36820149 |
| chr18 | 36999236 | 36999368 |
| chr18 | 37185198 | 37185344 |
| chr18 | 37242424 | 37242550 |
| chr18 | 37430932 | 37431054 |
| chr18 | 37590153 | 37590305 |
| chr18 | 37773812 | 37773940 |
| chr18 | 37874875 | 37874999 |
| chr18 | 38051484 | 38051610 |
| chr18 | 38173879 | 38174017 |
| chr18 | 38344787 | 38344925 |
| chr18 | 38422236 | 38422366 |
| chr18 | 38643318 | 38643456 |
| chr18 | 38778797 | 38778925 |
| chr18 | 39005437 | 39005577 |
| chr18 | 39074228 | 39074350 |
| chr18 | 39178573 | 39178709 |
| chr18 | 39399207 | 39399349 |
| chr18 | 39580404 | 39580536 |
| chr18 | 39646177 | 39646307 |
| chr18 | 39801016 | 39801164 |
| chr18 | 39971620 | 39971744 |
| chr18 | 40092647 | 40092801 |
| chr18 | 40337501 | 40337629 |
| chr18 | 40504314 | 40504446 |
| chr18 | 40643193 | 40643337 |
| chr18 | 40731804 | 40731942 |
| chr18 | 40846735 | 40846857 |
| chr18 | 41015077 | 41015201 |
| chr18 | 41148417 | 41148541 |
| chr18 | 41339207 | 41339345 |
| chr18 | 41538070 | 41538192 |
| chr18 | 41661346 | 41661470 |
| chr18 | 41809629 | 41809785 |
| chr18 | 41899173 | 41899325 |
| chr18 | 42141919 | 42142043 |
| chr18 | 42166068 | 42166216 |
| chr18 | 42442872 | 42443012 |
| chr18 | 42600646 | 42600774 |
| chr18 | 42667882 | 42668006 |
| chr18 | 42903665 | 42903791 |
| chr18 | 42970357 | 42970501 |
| chr18 | 43176252 | 43176376 |
| chr18 | 43410306 | 43410428 |
| chr18 | 43633229 | 43633361 |
| chr18 | 43782220 | 43782348 |
| chr18 | 43817699 | 43817827 |
| chr18 | 44027141 | 44027265 |
| chr18 | 44116060 | 44116190 |
| chr18 | 44263747 | 44263875 |
| chr18 | 44431774 | 44431912 |
| chr18 | 44562730 | 44562851 |
| chr18 | 44758589 | 44758713 |
| chr18 | 44884162 | 44884300 |
| chr18 | 45051978 | 45052104 |
| chr18 | 45179847 | 45179971 |
| chr18 | 45434626 | 45434748 |
| chr18 | 45470131 | 45470261 |
| chr18 | 45621481 | 45621621 |
| chr18 | 45787458 | 45787584 |
| chr18 | 45955729 | 45955851 |
| chr18 | 46198434 | 46198568 |
| chr18 | 46294218 | 46294340 |
| chr18 | 46417033 | 46417157 |
| chr18 | 46605375 | 46605502 |
| chr18 | 46712746 | 46712876 |
| chr18 | 46929058 | 46929254 |
| chr18 | 46965197 | 46965514 |
| chr18 | 47187925 | 47188065 |
| chr18 | 47293656 | 47293788 |
| chr18 | 47541522 | 47541648 |
| chr18 | 47663567 | 47663691 |
| chr18 | 47724995 | 47725125 |
| chr18 | 47936161 | 47936311 |
| chr18 | 48092899 | 48093033 |
| chr18 | 48262332 | 48262454 |
| chr18 | 48392464 | 48392598 |
| chr18 | 48552576 | 48552855 |
| chr18 | 48741265 | 48741387 |
| chr18 | 48823200 | 48823338 |
| chr18 | 48996366 | 48996488 |
| chr18 | 49087767 | 49087899 |
| chr18 | 49322396 | 49322526 |
| chr18 | 49423095 | 49423219 |
| chr18 | 49605407 | 49605531 |
| chr18 | 49728317 | 49728449 |
| chr18 | 49930636 | 49930762 |
| chr18 | 50060305 | 50060447 |
| chr18 | 50180531 | 50180655 |
| chr18 | 50334282 | 50334408 |
| chr18 | 50490785 | 50490907 |
| chr18 | 50603437 | 50603581 |
| chr18 | 50723197 | 50723321 |
| chr18 | 51010123 | 51010265 |
| chr18 | 51138900 | 51139036 |
| chr18 | 51284693 | 51284817 |
| chr18 | 51401357 | 51401487 |
| chr18 | 51485053 | 51485187 |
| chr18 | 51703129 | 51703251 |
| chr18 | 51864382 | 51864504 |
| chr18 | 51932737 | 51932859 |
| chr18 | 51997771 | 51997897 |
| chr18 | 52058307 | 52058459 |
| chr18 | 52212030 | 52212150 |
| chr18 | 52268591 | 52268713 |
| chr18 | 52410433 | 52410561 |
| chr18 | 52528626 | 52528758 |
| chr18 | 52777208 | 52777342 |
| chr18 | 52938309 | 52938451 |
| chr18 | 52966935 | 52967147 |
| chr18 | 53228165 | 53228286 |
| chr18 | 53287530 | 53287652 |
| chr18 | 53449585 | 53449715 |
| chr18 | 53606451 | 53606605 |
| chr18 | 53760575 | 53760717 |
| chr18 | 53966727 | 53966851 |
| chr18 | 54078013 | 54078139 |
| chr18 | 54171071 | 54171227 |
| chr18 | 54404478 | 54404606 |
| chr18 | 54549506 | 54549644 |
| chr18 | 54654217 | 54654357 |
| chr18 | 54822485 | 54822621 |
| chr18 | 55043698 | 55043830 |
| chr18 | 55183886 | 55184016 |
| chr18 | 55314171 | 55314295 |
| chr18 | 55449464 | 55449586 |
| chr18 | 55569192 | 55569334 |
| chr18 | 55715596 | 55715722 |
| chr18 | 55846399 | 55846529 |
| chr18 | 55968562 | 55968684 |
| chr18 | 56256382 | 56256506 |
| chr18 | 56367176 | 56367298 |
| chr18 | 56499982 | 56500134 |
| chr18 | 56582923 | 56583053 |
| chr18 | 56759160 | 56759290 |
| chr18 | 56953409 | 56953549 |
| chr18 | 57086891 | 57087023 |
| chr18 | 57283181 | 57283305 |
| chr18 | 57445985 | 57446129 |
| chr18 | 57511512 | 57511650 |
| chr18 | 57720310 | 57720444 |
| chr18 | 57789986 | 57790136 |
| chr18 | 58044095 | 58044217 |
| chr18 | 58067759 | 58068049 |
| chr18 | 58310289 | 58310417 |
| chr18 | 58468813 | 58468937 |
| chr18 | 58557156 | 58557278 |
| chr18 | 58800284 | 58800428 |
| chr18 | 58850691 | 58850819 |
| chr18 | 59068800 | 59068924 |
| chr18 | 59159718 | 59159846 |
| chr18 | 59295922 | 59296056 |
| chr18 | 59501696 | 59501822 |
| chr18 | 59607497 | 59607633 |
| chr18 | 59819930 | 59820058 |
| chr18 | 59994714 | 59994870 |
| chr18 | 60151925 | 60152057 |
| chr18 | 60194800 | 60194932 |
| chr18 | 60454703 | 60454837 |
| chr18 | 60593880 | 60594004 |
| chr18 | 60661132 | 60661278 |
| chr18 | 60837756 | 60837888 |
| chr18 | 60978931 | 60979063 |
| chr18 | 61179734 | 61179858 |
| chr18 | 61245761 | 61245901 |
| chr18 | 61498360 | 61498484 |
| chr18 | 61633168 | 61633294 |
| chr18 | 61722847 | 61722971 |
| chr18 | 61864204 | 61864338 |
| chr18 | 61985378 | 61985514 |
| chr18 | 62185339 | 62185467 |
| chr18 | 62396752 | 62396880 |
| chr18 | 62458624 | 62458760 |
| chr18 | 62626759 | 62626887 |
| chr18 | 62856692 | 62856816 |
| chr18 | 62979360 | 62979514 |
| chr18 | 63058636 | 63058786 |
| chr18 | 63270021 | 63270163 |
| chr18 | 63409395 | 63409543 |
| chr18 | 63464991 | 63465115 |
| chr18 | 63691676 | 63691798 |
| chr18 | 63798962 | 63799098 |
| chr18 | 64009047 | 64009171 |
| chr18 | 64086708 | 64086848 |
| chr18 | 64321050 | 64321190 |
| chr18 | 64480120 | 64480256 |
| chr18 | 64645196 | 64645318 |
| chr18 | 64741772 | 64741900 |
| chr18 | 64950974 | 64951096 |
| chr18 | 65046085 | 65046211 |
| chr18 | 65241848 | 65241994 |
| chr18 | 65371251 | 65371373 |
| chr18 | 65421618 | 65421754 |
| chr18 | 65630536 | 65630658 |
| chr18 | 65724686 | 65724814 |
| chr18 | 65887249 | 65887373 |
| chr18 | 66132755 | 66132887 |
| chr18 | 66272526 | 66272650 |
| chr18 | 66374040 | 66374194 |
| chr18 | 66553860 | 66554000 |
| chr18 | 66647996 | 66648252 |
| chr18 | 66871683 | 66871807 |
| chr18 | 67017673 | 67017801 |
| chr18 | 67169906 | 67170028 |
| chr18 | 67345843 | 67345987 |
| chr18 | 67465517 | 67465643 |
| chr18 | 67543602 | 67543724 |
| chr18 | 67692703 | 67692976 |
| chr18 | 67847201 | 67847321 |
| chr18 | 67982721 | 67982853 |
| chr18 | 68250281 | 68250423 |
| chr18 | 68365715 | 68365843 |
| chr18 | 68537451 | 68537577 |
| chr18 | 68703419 | 68703541 |
| chr18 | 68799306 | 68799432 |
| chr18 | 68925978 | 68926100 |
| chr18 | 69037734 | 69037860 |
| chr18 | 69297544 | 69297692 |
| chr18 | 69417441 | 69417565 |
| chr18 | 69487850 | 69487984 |
| chr18 | 69738839 | 69738963 |
| chr18 | 69762210 | 69762340 |
| chr18 | 69940461 | 69940585 |
| chr18 | 70107367 | 70107491 |
| chr18 | 70270008 | 70270136 |
| chr18 | 70440285 | 70440539 |
| chr18 | 70560528 | 70560656 |
| chr18 | 70674460 | 70674581 |
| chr18 | 70869484 | 70869605 |
| chr18 | 70990967 | 70991103 |
| chr18 | 71125639 | 71125773 |
| chr18 | 71303621 | 71303743 |
| chr18 | 71457347 | 71457473 |
| chr18 | 71639343 | 71639475 |
| chr18 | 71820263 | 71820385 |
| chr18 | 71945240 | 71945380 |
| chr18 | 72148297 | 72148421 |
| chr18 | 72196224 | 72196358 |
| chr18 | 72414831 | 72414961 |
| chr18 | 72481245 | 72481371 |
| chr18 | 72712071 | 72712195 |
| chr18 | 72852921 | 72853043 |
| chr18 | 73043433 | 73043561 |
| chr18 | 73122042 | 73122202 |
| chr18 | 73340548 | 73340672 |
| chr18 | 73387487 | 73387609 |
| chr18 | 73652379 | 73652517 |
| chr18 | 73730695 | 73730821 |
| chr18 | 73915103 | 73915247 |
| chr18 | 74026387 | 74026517 |
| chr18 | 74253191 | 74253339 |
| chr18 | 74304685 | 74304815 |
| chr18 | 74419345 | 74419471 |
| chr18 | 74598346 | 74598492 |
| chr18 | 74827327 | 74827469 |
| chr18 | 74974195 | 74974341 |
| chr18 | 75073537 | 75073661 |
| chr18 | 75165292 | 75165424 |
| chr18 | 75347077 | 75347203 |
| chr18 | 75479105 | 75479237 |
| chr18 | 75664497 | 75664635 |
| chr18 | 75778690 | 75778823 |
| chr18 | 75937906 | 75938034 |
| chr18 | 76089953 | 76090075 |
| chr18 | 76337558 | 76337684 |
| chr18 | 76447105 | 76447233 |
| chr18 | 76634784 | 76634910 |
| chr18 | 76773925 | 76774061 |
| chr18 | 76922857 | 76922985 |
| chr18 | 77050809 | 77050963 |
| chr18 | 77220608 | 77220732 |
| chr18 | 77314352 | 77314476 |
| chr18 | 77429639 | 77429783 |
| chr18 | 77595581 | 77595735 |
| chr18 | 77848152 | 77848276 |
| chr18 | 77931002 | 77931148 |
| chr18 | 78012887 | 78013015 |
| chr19 | 229052 | 229182 |
| chr19 | 239731 | 239853 |
| chr19 | 243936 | 244066 |
| chr19 | 260876 | 261000 |
| chr19 | 267003 | 267127 |
| chr19 | 277661 | 277797 |
| chr19 | 288313 | 288443 |
| chr19 | 328779 | 329042 |
| chr19 | 397059 | 397181 |
| chr19 | 680793 | 680927 |
| chr19 | 823464 | 823638 |
| chr19 | 986735 | 986861 |
| chr19 | 1075985 | 1076111 |
| chr19 | 1204613 | 1204733 |
| chr19 | 1384612 | 1384746 |
| chr19 | 1586129 | 1586277 |
| chr19 | 1741579 | 1741721 |
| chr19 | 1848903 | 1849045 |
| chr19 | 2006201 | 2006323 |
| chr19 | 2053932 | 2054076 |
| chr19 | 2258133 | 2258257 |
| chr19 | 2363251 | 2363405 |
| chr19 | 2523691 | 2523815 |
| chr19 | 2781267 | 2781397 |
| chr19 | 2923094 | 2923240 |
| chr19 | 3090798 | 3090934 |
| chr19 | 3165843 | 3165983 |
| chr19 | 3285125 | 3285247 |
| chr19 | 3497152 | 3497282 |
| chr19 | 3685072 | 3685204 |
| chr19 | 3796781 | 3796923 |
| chr19 | 3889627 | 3889751 |
| chr19 | 4118847 | 4118987 |
| chr19 | 4147387 | 4147533 |
| chr19 | 4342769 | 4342897 |
| chr19 | 4540627 | 4540753 |
| chr19 | 4617692 | 4617858 |
| chr19 | 4830544 | 4830678 |
| chr19 | 4912477 | 4912645 |
| chr19 | 5142729 | 5142851 |
| chr19 | 5200853 | 5200985 |
| chr19 | 5346822 | 5346974 |
| chr19 | 5606169 | 5606309 |
| chr19 | 5644107 | 5644233 |
| chr19 | 5814622 | 5814754 |
| chr19 | 6088776 | 6088916 |
| chr19 | 6147153 | 6147275 |
| chr19 | 6363457 | 6363579 |
| chr19 | 6522044 | 6522196 |
| chr19 | 6556690 | 6556826 |
| chr19 | 6788879 | 6789025 |
| chr19 | 6895790 | 6895914 |
| chr19 | 7013897 | 7014028 |
| chr19 | 7208152 | 7208276 |
| chr19 | 7296209 | 7296341 |
| chr19 | 7480836 | 7480966 |
| chr19 | 7723407 | 7723533 |
| chr19 | 7846775 | 7846905 |
| chr19 | 7903404 | 7903532 |
| chr19 | 8162613 | 8162763 |
| chr19 | 8223377 | 8223499 |
| chr19 | 8483222 | 8483368 |
| chr19 | 8570051 | 8570189 |
| chr19 | 8682999 | 8683120 |
| chr19 | 8838364 | 8838650 |
| chr19 | 9078134 | 9078272 |
| chr19 | 9172174 | 9172324 |
| chr19 | 9310300 | 9310424 |
| chr19 | 9493129 | 9493255 |
| chr19 | 9601073 | 9601219 |
| chr19 | 9783768 | 9783895 |
| chr19 | 9982278 | 9982420 |
| chr19 | 10028209 | 10028349 |
| chr19 | 10235047 | 10235169 |
| chr19 | 10323624 | 10323754 |
| chr19 | 10560870 | 10560992 |
| chr19 | 10643262 | 10643386 |
| chr19 | 10889411 | 10889555 |
| chr19 | 11029236 | 11029370 |
| chr19 | 11065357 | 11065487 |
| chr19 | 11302741 | 11302883 |
| chr19 | 11401063 | 11401187 |
| chr19 | 11615458 | 11615590 |
| chr19 | 11757927 | 11758051 |
| chr19 | 11871092 | 11871218 |
| chr19 | 11994806 | 11994936 |
| chr19 | 12242402 | 12242530 |
| chr19 | 12393877 | 12394029 |
| chr19 | 12487193 | 12487361 |
| chr19 | 12607877 | 12608001 |
| chr19 | 12842951 | 12843072 |
| chr19 | 12928259 | 12928516 |
| chr19 | 13131782 | 13131922 |
| chr19 | 13221784 | 13221920 |
| chr19 | 13385343 | 13385497 |
| chr19 | 13539512 | 13539666 |
| chr19 | 13730204 | 13730328 |
| chr19 | 13887601 | 13887729 |
| chr19 | 13951754 | 13951898 |
| chr19 | 14170387 | 14170509 |
| chr19 | 14282298 | 14282420 |
| chr19 | 14456396 | 14456520 |
| chr19 | 14494421 | 14494547 |
| chr19 | 14647420 | 14647570 |
| chr19 | 14820606 | 14820734 |
| chr19 | 15019493 | 15019617 |
| chr19 | 15240753 | 15240885 |
| chr19 | 15307275 | 15307403 |
| chr19 | 15440758 | 15441010 |
| chr19 | 15621451 | 15621573 |
| chr19 | 15701852 | 15701974 |
| chr19 | 15848738 | 15848866 |
| chr19 | 16057621 | 16057753 |
| chr19 | 16234459 | 16234597 |
| chr19 | 16439454 | 16439586 |
| chr19 | 16580460 | 16580586 |
| chr19 | 16694574 | 16694696 |
| chr19 | 16760989 | 16761137 |
| chr19 | 17030606 | 17030756 |
| chr19 | 17061996 | 17062136 |
| chr19 | 17341850 | 17341990 |
| chr19 | 17467142 | 17467268 |
| chr19 | 17574819 | 17574967 |
| chr19 | 17700898 | 17701028 |
| chr19 | 17829548 | 17829688 |
| chr19 | 18041161 | 18041429 |
| chr19 | 18181204 | 18181334 |
| chr19 | 18341676 | 18341798 |
| chr19 | 18407585 | 18407727 |
| chr19 | 18624243 | 18624381 |
| chr19 | 18772533 | 18772661 |
| chr19 | 18867473 | 18867611 |
| chr19 | 19123960 | 19124118 |
| chr19 | 19216638 | 19216790 |
| chr19 | 19328324 | 19328449 |
| chr19 | 19457834 | 19457966 |
| chr19 | 19631567 | 19631705 |
| chr19 | 19884516 | 19884656 |
| chr19 | 20008967 | 20009145 |
| chr19 | 20091624 | 20091758 |
| chr19 | 20234588 | 20234732 |
| chr19 | 20476963 | 20477087 |
| chr19 | 20639692 | 20639846 |
| chr19 | 20716620 | 20716741 |
| chr19 | 20898880 | 20899001 |
| chr19 | 21048988 | 21049130 |
| chr19 | 21218739 | 21218873 |
| chr19 | 21378103 | 21378226 |
| chr19 | 21487394 | 21487524 |
| chr19 | 21676135 | 21676259 |
| chr19 | 21800951 | 21801073 |
| chr19 | 21976928 | 21977052 |
| chr19 | 22084130 | 22084260 |
| chr19 | 22215794 | 22215915 |
| chr19 | 22299951 | 22300083 |
| chr19 | 22512456 | 22512606 |
| chr19 | 22678621 | 22678761 |
| chr19 | 22826456 | 22826721 |
| chr19 | 23017244 | 23017384 |
| chr19 | 23150381 | 23150509 |
| chr19 | 23232413 | 23232569 |
| chr19 | 23490141 | 23490277 |
| chr19 | 23568342 | 23568488 |
| chr19 | 23753939 | 23754073 |
| chr19 | 23847102 | 23847224 |
| chr19 | 23960764 | 23960888 |
| chr19 | 24145083 | 24145225 |
| chr19 | 24344514 | 24344642 |
| chr19 | 24487824 | 24487945 |
| chr19 | 28016152 | 28016273 |
| chr19 | 28052300 | 28052426 |
| chr19 | 28138087 | 28138209 |
| chr19 | 28291374 | 28291524 |
| chr19 | 28417274 | 28417398 |
| chr19 | 28496839 | 28496967 |
| chr19 | 28690225 | 28690375 |
| chr19 | 28817961 | 28818083 |
| chr19 | 29010414 | 29010540 |
| chr19 | 29048563 | 29048709 |
| chr19 | 29238002 | 29238126 |
| chr19 | 29392690 | 29392832 |
| chr19 | 29573649 | 29573773 |
| chr19 | 29661529 | 29661673 |
| chr19 | 29912451 | 29912577 |
| chr19 | 30073254 | 30073378 |
| chr19 | 30162405 | 30162543 |
| chr19 | 30345444 | 30345604 |
| chr19 | 30394770 | 30394894 |
| chr19 | 30654244 | 30654366 |
| chr19 | 30784278 | 30784418 |
| chr19 | 30899200 | 30899334 |
| chr19 | 31018423 | 31018579 |
| chr19 | 31230634 | 31230758 |
| chr19 | 31401666 | 31401792 |
| chr19 | 31570661 | 31570933 |
| chr19 | 31611563 | 31611695 |
| chr19 | 31859208 | 31859336 |
| chr19 | 32017468 | 32017606 |
| chr19 | 32133859 | 32133983 |
| chr19 | 32234427 | 32234567 |
| chr19 | 32413123 | 32413369 |
| chr19 | 32588634 | 32588792 |
| chr19 | 32764558 | 32764706 |
| chr19 | 32824272 | 32824398 |
| chr19 | 33047039 | 33047171 |
| chr19 | 33199415 | 33199563 |
| chr19 | 33250654 | 33250778 |
| chr19 | 33400081 | 33400203 |
| chr19 | 33645667 | 33645797 |
| chr19 | 33757010 | 33757132 |
| chr19 | 33964091 | 33964215 |
| chr19 | 34084455 | 34084593 |
| chr19 | 34145309 | 34145449 |
| chr19 | 34346749 | 34346881 |
| chr19 | 34568046 | 34568190 |
| chr19 | 34667637 | 34667759 |
| chr19 | 34764511 | 34764633 |
| chr19 | 35041213 | 35041337 |
| chr19 | 35063250 | 35063374 |
| chr19 | 35243994 | 35244132 |
| chr19 | 35374242 | 35374366 |
| chr19 | 35557350 | 35557492 |
| chr19 | 35727954 | 35728096 |
| chr19 | 35865576 | 35865698 |
| chr19 | 36039632 | 36039766 |
| chr19 | 36198982 | 36199106 |
| chr19 | 36319668 | 36319792 |
| chr19 | 36407840 | 36408080 |
| chr19 | 36563673 | 36563797 |
| chr19 | 36721458 | 36721580 |
| chr19 | 36910926 | 36911062 |
| chr19 | 37088925 | 37089053 |
| chr19 | 37143946 | 37144080 |
| chr19 | 37315940 | 37316068 |
| chr19 | 37498174 | 37498310 |
| chr19 | 37649470 | 37649596 |
| chr19 | 37747044 | 37747196 |
| chr19 | 37988663 | 37988797 |
| chr19 | 38066670 | 38066796 |
| chr19 | 38293401 | 38293523 |
| chr19 | 38400250 | 38400372 |
| chr19 | 38497367 | 38497515 |
| chr19 | 38705088 | 38705216 |
| chr19 | 38909549 | 38909697 |
| chr19 | 39017296 | 39017466 |
| chr19 | 39162142 | 39162268 |
| chr19 | 39266221 | 39266371 |
| chr19 | 39500892 | 39501026 |
| chr19 | 39673946 | 39674096 |
| chr19 | 39791718 | 39791842 |
| chr19 | 39861458 | 39861602 |
| chr19 | 40024234 | 40024364 |
| chr19 | 40202328 | 40202468 |
| chr19 | 40333891 | 40334039 |
| chr19 | 40579788 | 40579912 |
| chr19 | 40609947 | 40610095 |
| chr19 | 40836964 | 40837112 |
| chr19 | 40963088 | 40963224 |
| chr19 | 41049822 | 41049960 |
| chr19 | 41333194 | 41333362 |
| chr19 | 41479233 | 41479357 |
| chr19 | 41528974 | 41529098 |
| chr19 | 41769022 | 41769176 |
| chr19 | 41804922 | 41805048 |
| chr19 | 41985527 | 41985675 |
| chr19 | 42130924 | 42131066 |
| chr19 | 42365878 | 42366018 |
| chr19 | 42428729 | 42428855 |
| chr19 | 42611894 | 42612158 |
| chr19 | 42780435 | 42780556 |
| chr19 | 42887367 | 42887641 |
| chr19 | 43074477 | 43074754 |
| chr19 | 43226917 | 43227085 |
| chr19 | 43291151 | 43291271 |
| chr19 | 43342325 | 43342445 |
| chr19 | 43415965 | 43416089 |
| chr19 | 43454917 | 43455065 |
| chr19 | 43512969 | 43513107 |
| chr19 | 43673035 | 43673203 |
| chr19 | 43726992 | 43727112 |
| chr19 | 43802703 | 43802826 |
| chr19 | 43973600 | 43973768 |
| chr19 | 44133588 | 44133716 |
| chr19 | 44256300 | 44256434 |
| chr19 | 44456632 | 44456758 |
| chr19 | 44565339 | 44565463 |
| chr19 | 44712616 | 44712756 |
| chr19 | 44930278 | 44930418 |
| chr19 | 44986950 | 44987074 |
| chr19 | 45161530 | 45161654 |
| chr19 | 45277543 | 45277799 |
| chr19 | 45519036 | 45519158 |
| chr19 | 45653144 | 45653302 |
| chr19 | 45767160 | 45767298 |
| chr19 | 45981403 | 45981525 |
| chr19 | 45995275 | 45995401 |
| chr19 | 46149796 | 46149920 |
| chr19 | 46302286 | 46302422 |
| chr19 | 46559962 | 46560086 |
| chr19 | 46594082 | 46594233 |
| chr19 | 46885915 | 46886059 |
| chr19 | 46984153 | 46984297 |
| chr19 | 47083170 | 47083300 |
| chr19 | 47265888 | 47266046 |
| chr19 | 47446601 | 47446725 |
| chr19 | 47585180 | 47585314 |
| chr19 | 47689865 | 47690005 |
| chr19 | 47815659 | 47815797 |
| chr19 | 48086791 | 48086945 |
| chr19 | 48218473 | 48218605 |
| chr19 | 48279811 | 48279981 |
| chr19 | 48486786 | 48486928 |
| chr19 | 48659229 | 48659359 |
| chr19 | 48711438 | 48711564 |
| chr19 | 48951803 | 48951931 |
| chr19 | 49018228 | 49018489 |
| chr19 | 49287691 | 49287859 |
| chr19 | 49396681 | 49396807 |
| chr19 | 49583592 | 49583720 |
| chr19 | 49702242 | 49702372 |
| chr19 | 49828777 | 49828903 |
| chr19 | 49991699 | 49991853 |
| chr19 | 50153244 | 50153368 |
| chr19 | 50238798 | 50238930 |
| chr19 | 50477609 | 50477759 |
| chr19 | 50540389 | 50540531 |
| chr19 | 50651655 | 50651846 |
| chr19 | 50896324 | 50896446 |
| chr19 | 51026099 | 51026221 |
| chr19 | 51095579 | 51095707 |
| chr19 | 51332391 | 51332515 |
| chr19 | 51455315 | 51455447 |
| chr19 | 51585169 | 51585313 |
| chr19 | 51838568 | 51838696 |
| chr19 | 51967797 | 51967947 |
| chr19 | 52126973 | 52127109 |
| chr19 | 52189868 | 52189996 |
| chr19 | 52301822 | 52301946 |
| chr19 | 52580714 | 52580838 |
| chr19 | 52670849 | 52670993 |
| chr19 | 52765891 | 52766015 |
| chr19 | 52921846 | 52921980 |
| chr19 | 53177289 | 53177410 |
| chr19 | 53243414 | 53243538 |
| chr19 | 53490453 | 53490581 |
| chr19 | 53557495 | 53557625 |
| chr19 | 53746487 | 53746641 |
| chr19 | 53892724 | 53892846 |
| chr19 | 54034509 | 54034655 |
| chr19 | 54180166 | 54180308 |
| chr19 | 54351881 | 54352025 |
| chr19 | 54518345 | 54518485 |
| chr19 | 54645747 | 54645869 |
| chr19 | 54754017 | 54754153 |
| chr19 | 54910444 | 54910592 |
| chr19 | 55013699 | 55013847 |
| chr19 | 55234714 | 55234835 |
| chr19 | 55398183 | 55398323 |
| chr19 | 55503713 | 55503835 |
| chr19 | 55684112 | 55684236 |
| chr19 | 55839184 | 55839308 |
| chr19 | 55913642 | 55913766 |
| chr19 | 56193598 | 56193732 |
| chr19 | 56259610 | 56259764 |
| chr19 | 56451542 | 56451700 |
| chr19 | 56647890 | 56648012 |
| chr19 | 56863262 | 56863394 |
| chr19 | 57063600 | 57063726 |
| chr19 | 57158112 | 57158242 |
| chr19 | 57361566 | 57361690 |
| chr19 | 57449241 | 57449375 |
| chr19 | 57601470 | 57601612 |
| chr19 | 57720085 | 57720215 |
| chr19 | 57946466 | 57946592 |
| chr19 | 57997762 | 57997886 |
| chr19 | 58252376 | 58252524 |
| chr19 | 58443283 | 58443411 |
| chr19 | 58548670 | 58548794 |
| chr19 | 58604907 | 58605071 |
| chr19 | 58823955 | 58824148 |
| chr19 | 59002656 | 59002784 |
| chr19 | 59057589 | 59057802 |
| chr19 | 59099542 | 59099672 |
| chr20 | 127525 | 127887 |
| chr20 | 344541 | 344665 |
| chr20 | 467312 | 467436 |
| chr20 | 577928 | 578082 |
| chr20 | 716175 | 716319 |
| chr20 | 830375 | 830499 |
| chr20 | 1009180 | 1009310 |
| chr20 | 1129967 | 1130091 |
| chr20 | 1409667 | 1409809 |
| chr20 | 1472334 | 1472486 |
| chr20 | 1636892 | 1637020 |
| chr20 | 1739030 | 1739154 |
| chr20 | 1897390 | 1897512 |
| chr20 | 2039137 | 2039289 |
| chr20 | 2198576 | 2198700 |
| chr20 | 2327059 | 2327191 |
| chr20 | 2520366 | 2520490 |
| chr20 | 2703602 | 2703734 |
| chr20 | 2870209 | 2870343 |
| chr20 | 3061362 | 3061486 |
| chr20 | 3170082 | 3170226 |
| chr20 | 3267028 | 3267154 |
| chr20 | 3375265 | 3375387 |
| chr20 | 3604718 | 3604842 |
| chr20 | 3666802 | 3666926 |
| chr20 | 3866429 | 3866571 |
| chr20 | 4103983 | 4104107 |
| chr20 | 4247016 | 4247174 |
| chr20 | 4305254 | 4305378 |
| chr20 | 4449626 | 4449750 |
| chr20 | 4643219 | 4643347 |
| chr20 | 4812896 | 4813024 |
| chr20 | 4942534 | 4942662 |
| chr20 | 5145629 | 5145751 |
| chr20 | 5198419 | 5198543 |
| chr20 | 5408503 | 5408647 |
| chr20 | 5515802 | 5515926 |
| chr20 | 5684372 | 5684512 |
| chr20 | 5851668 | 5851794 |
| chr20 | 5973460 | 5973586 |
| chr20 | 6123390 | 6123540 |
| chr20 | 6262133 | 6262263 |
| chr20 | 6412528 | 6412662 |
| chr20 | 6657518 | 6657640 |
| chr20 | 6746408 | 6746540 |
| chr20 | 6960474 | 6960598 |
| chr20 | 7057739 | 7057863 |
| chr20 | 7118518 | 7118644 |
| chr20 | 7352159 | 7352285 |
| chr20 | 7529657 | 7529785 |
| chr20 | 7673602 | 7673746 |
| chr20 | 7777410 | 7777548 |
| chr20 | 7885106 | 7885240 |
| chr20 | 8120777 | 8120915 |
| chr20 | 8179317 | 8179441 |
| chr20 | 8389837 | 8389959 |
| chr20 | 8592956 | 8593092 |
| chr20 | 8711605 | 8711763 |
| chr20 | 8881508 | 8881638 |
| chr20 | 9011860 | 9012002 |
| chr20 | 9091149 | 9091303 |
| chr20 | 9307159 | 9307311 |
| chr20 | 9466474 | 9466612 |
| chr20 | 9516054 | 9516178 |
| chr20 | 9723477 | 9723625 |
| chr20 | 9901301 | 9901469 |
| chr20 | 10045026 | 10045158 |
| chr20 | 10254421 | 10254559 |
| chr20 | 10320168 | 10320300 |
| chr20 | 10418745 | 10418871 |
| chr20 | 10694435 | 10694559 |
| chr20 | 10846869 | 10846999 |
| chr20 | 10863424 | 10863550 |
| chr20 | 11079401 | 11079561 |
| chr20 | 11265045 | 11265173 |
| chr20 | 11383249 | 11383379 |
| chr20 | 11518390 | 11518516 |
| chr20 | 11677728 | 11677850 |
| chr20 | 11908059 | 11908183 |
| chr20 | 12011211 | 12011339 |
| chr20 | 12157831 | 12157955 |
| chr20 | 12316072 | 12316196 |
| chr20 | 12435666 | 12435790 |
| chr20 | 12602384 | 12602528 |
| chr20 | 12707946 | 12708070 |
| chr20 | 12874341 | 12874481 |
| chr20 | 13004534 | 13004680 |
| chr20 | 13244045 | 13244169 |
| chr20 | 13355392 | 13355518 |
| chr20 | 13527845 | 13527983 |
| chr20 | 13612123 | 13612245 |
| chr20 | 13788802 | 13788938 |
| chr20 | 13973950 | 13974078 |
| chr20 | 14051283 | 14051409 |
| chr20 | 14163498 | 14163622 |
| chr20 | 14428046 | 14428170 |
| chr20 | 14595637 | 14595779 |
| chr20 | 14732549 | 14732673 |
| chr20 | 14889935 | 14890081 |
| chr20 | 15027578 | 15027718 |
| chr20 | 15208317 | 15208449 |
| chr20 | 15246950 | 15247072 |
| chr20 | 15492306 | 15492428 |
| chr20 | 15588556 | 15588680 |
| chr20 | 15788084 | 15788214 |
| chr20 | 15869219 | 15869343 |
| chr20 | 16109715 | 16109839 |
| chr20 | 16259041 | 16259175 |
| chr20 | 16392068 | 16392212 |
| chr20 | 16547508 | 16547638 |
| chr20 | 16683722 | 16683848 |
| chr20 | 16789719 | 16789877 |
| chr20 | 16952267 | 16952403 |
| chr20 | 17052007 | 17052133 |
| chr20 | 17254208 | 17254330 |
| chr20 | 17331156 | 17331280 |
| chr20 | 17552259 | 17552389 |
| chr20 | 17690441 | 17690575 |
| chr20 | 17895322 | 17895464 |
| chr20 | 18047680 | 18047812 |
| chr20 | 18199280 | 18199418 |
| chr20 | 18330979 | 18331109 |
| chr20 | 18459344 | 18459474 |
| chr20 | 18598629 | 18598771 |
| chr20 | 18769562 | 18769686 |
| chr20 | 18911278 | 18911434 |
| chr20 | 19067051 | 19067187 |
| chr20 | 19228149 | 19228291 |
| chr20 | 19358768 | 19358910 |
| chr20 | 19523508 | 19523634 |
| chr20 | 19630699 | 19630853 |
| chr20 | 19839153 | 19839283 |
| chr20 | 19873786 | 19873910 |
| chr20 | 20126995 | 20127135 |
| chr20 | 20222938 | 20223066 |
| chr20 | 20411638 | 20411782 |
| chr20 | 20483127 | 20483257 |
| chr20 | 20753454 | 20753594 |
| chr20 | 20766174 | 20766312 |
| chr20 | 21049320 | 21049450 |
| chr20 | 21182733 | 21182855 |
| chr20 | 21266554 | 21266678 |
| chr20 | 21408653 | 21408801 |
| chr20 | 21552462 | 21552586 |
| chr20 | 21732017 | 21732163 |
| chr20 | 21813981 | 21814105 |
| chr20 | 21999161 | 21999285 |
| chr20 | 22192380 | 22192534 |
| chr20 | 22327037 | 22327171 |
| chr20 | 22439722 | 22439880 |
| chr20 | 22654077 | 22654201 |
| chr20 | 22845250 | 22845372 |
| chr20 | 22954240 | 22954396 |
| chr20 | 23160176 | 23160304 |
| chr20 | 23229382 | 23229506 |
| chr20 | 23441974 | 23442120 |
| chr20 | 23487511 | 23487635 |
| chr20 | 23627302 | 23627436 |
| chr20 | 23851899 | 23852023 |
| chr20 | 24052817 | 24052941 |
| chr20 | 24145650 | 24145786 |
| chr20 | 24344821 | 24344971 |
| chr20 | 24499090 | 24499228 |
| chr20 | 24660215 | 24660363 |
| chr20 | 24707832 | 24707974 |
| chr20 | 24820525 | 24820647 |
| chr20 | 25035631 | 25035759 |
| chr20 | 25232258 | 25232388 |
| chr20 | 25291780 | 25291902 |
| chr20 | 25532353 | 25532489 |
| chr20 | 25608994 | 25609120 |
| chr20 | 25725219 | 25725345 |
| chr20 | 26112426 | 26112547 |
| chr20 | 29647038 | 29647164 |
| chr20 | 29876698 | 29876838 |
| chr20 | 30004603 | 30004725 |
| chr20 | 30161822 | 30161948 |
| chr20 | 30336597 | 30336727 |
| chr20 | 30372024 | 30372148 |
| chr20 | 30629958 | 30630082 |
| chr20 | 30779040 | 30779166 |
| chr20 | 30844496 | 30844620 |
| chr20 | 30988008 | 30988172 |
| chr20 | 31229326 | 31229480 |
| chr20 | 31348660 | 31348838 |
| chr20 | 31539022 | 31539158 |
| chr20 | 31652726 | 31652904 |
| chr20 | 31861583 | 31861723 |
| chr20 | 31974307 | 31974429 |
| chr20 | 32084448 | 32084697 |
| chr20 | 32298952 | 32299078 |
| chr20 | 32425343 | 32425489 |
| chr20 | 32564096 | 32564218 |
| chr20 | 32738634 | 32738768 |
| chr20 | 32783423 | 32783575 |
| chr20 | 33017972 | 33018096 |
| chr20 | 33180566 | 33180696 |
| chr20 | 33322784 | 33322922 |
| chr20 | 33488685 | 33488807 |
| chr20 | 33549229 | 33549399 |
| chr20 | 33713605 | 33713727 |
| chr20 | 33948875 | 33948999 |
| chr20 | 34007174 | 34007657 |
| chr20 | 34233226 | 34233352 |
| chr20 | 34322382 | 34322506 |
| chr20 | 34425896 | 34426056 |
| chr20 | 34688429 | 34688577 |
| chr20 | 34822239 | 34822367 |
| chr20 | 34891514 | 34891638 |
| chr20 | 35029615 | 35029757 |
| chr20 | 35179456 | 35179580 |
| chr20 | 35352644 | 35352768 |
| chr20 | 35500620 | 35500744 |
| chr20 | 35743055 | 35743191 |
| chr20 | 35786486 | 35786610 |
| chr20 | 35925206 | 35925348 |
| chr20 | 36156968 | 36157401 |
| chr20 | 36321282 | 36321410 |
| chr20 | 36404868 | 36405004 |
| chr20 | 36619395 | 36619535 |
| chr20 | 36811327 | 36811449 |
| chr20 | 36900250 | 36900378 |
| chr20 | 37018779 | 37018921 |
| chr20 | 37217746 | 37217867 |
| chr20 | 37310434 | 37310556 |
| chr20 | 37424201 | 37424331 |
| chr20 | 37666578 | 37666726 |
| chr20 | 37859476 | 37859600 |
| chr20 | 37971940 | 37972080 |
| chr20 | 38096631 | 38096769 |
| chr20 | 38225462 | 38225596 |
| chr20 | 38454103 | 38454249 |
| chr20 | 38584872 | 38585020 |
| chr20 | 38710356 | 38710480 |
| chr20 | 38823907 | 38824043 |
| chr20 | 38997008 | 38997136 |
| chr20 | 39204574 | 39204730 |
| chr20 | 39265649 | 39265793 |
| chr20 | 39366294 | 39366418 |
| chr20 | 39548770 | 39548908 |
| chr20 | 39709167 | 39709289 |
| chr20 | 39890263 | 39890389 |
| chr20 | 40066688 | 40066824 |
| chr20 | 40238126 | 40238248 |
| chr20 | 40335178 | 40335316 |
| chr20 | 40511731 | 40511865 |
| chr20 | 40709944 | 40710066 |
| chr20 | 40792026 | 40792182 |
| chr20 | 40971649 | 40971781 |
| chr20 | 41109885 | 41110009 |
| chr20 | 41202882 | 41203006 |
| chr20 | 41423977 | 41424113 |
| chr20 | 41609024 | 41609158 |
| chr20 | 41736124 | 41736266 |
| chr20 | 41814074 | 41814252 |
| chr20 | 41931559 | 41931689 |
| chr20 | 42175538 | 42175674 |
| chr20 | 42216565 | 42216711 |
| chr20 | 42430824 | 42430962 |
| chr20 | 42591152 | 42591310 |
| chr20 | 42752452 | 42752582 |
| chr20 | 42846504 | 42846628 |
| chr20 | 42989185 | 42989323 |
| chr20 | 43216429 | 43216555 |
| chr20 | 43405256 | 43405380 |
| chr20 | 43528892 | 43529034 |
| chr20 | 43611296 | 43611422 |
| chr20 | 43855940 | 43856084 |
| chr20 | 43928044 | 43928166 |
| chr20 | 44076475 | 44076599 |
| chr20 | 44265466 | 44265610 |
| chr20 | 44355168 | 44355294 |
| chr20 | 44466208 | 44466354 |
| chr20 | 44730709 | 44730835 |
| chr20 | 44813214 | 44813356 |
| chr20 | 44941919 | 44942045 |
| chr20 | 45101546 | 45101668 |
| chr20 | 45246755 | 45246879 |
| chr20 | 45404568 | 45404706 |
| chr20 | 45600253 | 45600385 |
| chr20 | 45665030 | 45665164 |
| chr20 | 45900084 | 45900234 |
| chr20 | 46093172 | 46093294 |
| chr20 | 46232809 | 46232935 |
| chr20 | 46329873 | 46329999 |
| chr20 | 46491869 | 46492003 |
| chr20 | 46621239 | 46621379 |
| chr20 | 46725347 | 46725477 |
| chr20 | 46915181 | 46915303 |
| chr20 | 47019964 | 47020088 |
| chr20 | 47228501 | 47228677 |
| chr20 | 47356429 | 47356571 |
| chr20 | 47565705 | 47565827 |
| chr20 | 47723071 | 47723211 |
| chr20 | 47786136 | 47786278 |
| chr20 | 48006225 | 48006349 |
| chr20 | 48115057 | 48115185 |
| chr20 | 48231057 | 48231189 |
| chr20 | 48369294 | 48369422 |
| chr20 | 48554925 | 48555047 |
| chr20 | 48733737 | 48733861 |
| chr20 | 48913886 | 48914022 |
| chr20 | 49081193 | 49081369 |
| chr20 | 49144589 | 49144717 |
| chr20 | 49324301 | 49324445 |
| chr20 | 49506125 | 49506281 |
| chr20 | 49671804 | 49671960 |
| chr20 | 49727607 | 49727747 |
| chr20 | 50004602 | 50004726 |
| chr20 | 50130459 | 50130583 |
| chr20 | 50189132 | 50189278 |
| chr20 | 50405898 | 50406020 |
| chr20 | 50566238 | 50566390 |
| chr20 | 50751092 | 50751238 |
| chr20 | 50899176 | 50899308 |
| chr20 | 50940504 | 50940642 |
| chr20 | 51133884 | 51134006 |
| chr20 | 51255166 | 51255290 |
| chr20 | 51393931 | 51394071 |
| chr20 | 51591848 | 51591976 |
| chr20 | 51675704 | 51675830 |
| chr20 | 51867943 | 51868067 |
| chr20 | 52080448 | 52080588 |
| chr20 | 52144205 | 52144333 |
| chr20 | 52338718 | 52338840 |
| chr20 | 52494645 | 52494769 |
| chr20 | 52693538 | 52693662 |
| chr20 | 52855784 | 52855926 |
| chr20 | 52973502 | 52973628 |
| chr20 | 53019479 | 53019609 |
| chr20 | 53290413 | 53290539 |
| chr20 | 53414453 | 53414597 |
| chr20 | 53490666 | 53490790 |
| chr20 | 53615331 | 53615469 |
| chr20 | 53854148 | 53854292 |
| chr20 | 53916848 | 53917017 |
| chr20 | 54162050 | 54162188 |
| chr20 | 54347392 | 54347516 |
| chr20 | 54422971 | 54423111 |
| chr20 | 54525142 | 54525264 |
| chr20 | 54746099 | 54746225 |
| chr20 | 54830614 | 54830736 |
| chr20 | 55067069 | 55067209 |
| chr20 | 55171289 | 55171415 |
| chr20 | 55294030 | 55294170 |
| chr20 | 55450723 | 55450851 |
| chr20 | 55671559 | 55671681 |
| chr20 | 55844599 | 55844721 |
| chr20 | 55997416 | 55997538 |
| chr20 | 56058673 | 56058803 |
| chr20 | 56298601 | 56298725 |
| chr20 | 56417643 | 56417787 |
| chr20 | 56463857 | 56463991 |
| chr20 | 56711082 | 56711206 |
| chr20 | 56873125 | 56873263 |
| chr20 | 56914157 | 56914285 |
| chr20 | 57133590 | 57133722 |
| chr20 | 57249501 | 57249825 |
| chr20 | 57448233 | 57448354 |
| chr20 | 57578340 | 57578480 |
| chr20 | 57714151 | 57714277 |
| chr20 | 57856935 | 57857083 |
| chr20 | 58062801 | 58062927 |
| chr20 | 58134103 | 58134229 |
| chr20 | 58267596 | 58267736 |
| chr20 | 58502390 | 58502530 |
| chr20 | 58673759 | 58673883 |
| chr20 | 58767280 | 58767424 |
| chr20 | 58916526 | 58916652 |
| chr20 | 59151910 | 59152042 |
| chr20 | 59197561 | 59197683 |
| chr20 | 59331235 | 59331361 |
| chr20 | 59485921 | 59486067 |
| chr20 | 59735633 | 59735755 |
| chr20 | 59804388 | 59804512 |
| chr20 | 59955746 | 59955874 |
| chr20 | 60180060 | 60180196 |
| chr20 | 60232623 | 60232761 |
| chr20 | 60394485 | 60394625 |
| chr20 | 60584696 | 60584818 |
| chr20 | 60680206 | 60680366 |
| chr20 | 60867707 | 60867839 |
| chr20 | 61008716 | 61008842 |
| chr20 | 61153316 | 61153438 |
| chr20 | 61360383 | 61360531 |
| chr20 | 61488218 | 61488348 |
| chr20 | 61625760 | 61625884 |
| chr20 | 61720003 | 61720137 |
| chr20 | 61966999 | 61967123 |
| chr20 | 62099296 | 62099426 |
| chr20 | 62286802 | 62286958 |
| chr20 | 62461259 | 62461383 |
| chr20 | 62529341 | 62529465 |
| chr20 | 62717840 | 62717964 |
| chr20 | 62873759 | 62873937 |
| chr21 | 9671274 | 9671394 |
| chr21 | 9828301 | 9828430 |
| chr21 | 9872236 | 9872368 |
| chr21 | 9916046 | 9916184 |
| chr21 | 9951219 | 9951339 |
| chr21 | 10001582 | 10001706 |
| chr21 | 10187534 | 10187678 |
| chr21 | 10369932 | 10370054 |
| chr21 | 10393291 | 10393421 |
| chr21 | 10399025 | 10399159 |
| chr21 | 10438138 | 10438260 |
| chr21 | 10491472 | 10491594 |
| chr21 | 10615040 | 10615168 |
| chr21 | 10861352 | 10861490 |
| chr21 | 10884182 | 10884304 |
| chr21 | 10887531 | 10887651 |
| chr21 | 11012420 | 11012548 |
| chr21 | 11083332 | 11083506 |
| chr21 | 11145162 | 11145297 |
| chr21 | 14397080 | 14397208 |
| chr21 | 14471385 | 14471543 |
| chr21 | 14490775 | 14490895 |
| chr21 | 14641154 | 14641300 |
| chr21 | 14983707 | 14983827 |
| chr21 | 15032666 | 15032786 |
| chr21 | 15050411 | 15050539 |
| chr21 | 15204105 | 15204241 |
| chr21 | 15407277 | 15407525 |
| chr21 | 15516303 | 15516431 |
| chr21 | 15629671 | 15630018 |
| chr21 | 15709941 | 15710079 |
| chr21 | 15861505 | 15861629 |
| chr21 | 16054603 | 16054755 |
| chr21 | 16193011 | 16193147 |
| chr21 | 16287987 | 16288129 |
| chr21 | 16431392 | 16431514 |
| chr21 | 16658018 | 16658150 |
| chr21 | 16753956 | 16754098 |
| chr21 | 16958843 | 16958969 |
| chr21 | 17166607 | 17166751 |
| chr21 | 17220529 | 17220653 |
| chr21 | 17382876 | 17383002 |
| chr21 | 17576475 | 17576613 |
| chr21 | 17774451 | 17774593 |
| chr21 | 17839632 | 17839899 |
| chr21 | 18038725 | 18038873 |
| chr21 | 18180950 | 18181086 |
| chr21 | 18261200 | 18261336 |
| chr21 | 18472187 | 18472359 |
| chr21 | 18632627 | 18632755 |
| chr21 | 18781251 | 18781373 |
| chr21 | 18875980 | 18876104 |
| chr21 | 19046687 | 19046811 |
| chr21 | 19153756 | 19153880 |
| chr21 | 19289290 | 19289414 |
| chr21 | 19544133 | 19544273 |
| chr21 | 19693243 | 19693387 |
| chr21 | 19840514 | 19840652 |
| chr21 | 19887318 | 19887446 |
| chr21 | 20039134 | 20039256 |
| chr21 | 20296830 | 20296976 |
| chr21 | 20442678 | 20442820 |
| chr21 | 20521645 | 20521769 |
| chr21 | 20671326 | 20671472 |
| chr21 | 20807307 | 20807431 |
| chr21 | 21047951 | 21048091 |
| chr21 | 21169008 | 21169152 |
| chr21 | 21337964 | 21338122 |
| chr21 | 21395522 | 21395644 |
| chr21 | 21644141 | 21644279 |
| chr21 | 21727440 | 21727568 |
| chr21 | 21957418 | 21957542 |
| chr21 | 22031178 | 22031324 |
| chr21 | 22263555 | 22263679 |
| chr21 | 22347875 | 22347997 |
| chr21 | 22502700 | 22502830 |
| chr21 | 22672014 | 22672156 |
| chr21 | 22770798 | 22770976 |
| chr21 | 22950907 | 22951033 |
| chr21 | 23169257 | 23169379 |
| chr21 | 23251181 | 23251315 |
| chr21 | 23470634 | 23470774 |
| chr21 | 23625549 | 23625697 |
| chr21 | 23719076 | 23719200 |
| chr21 | 23827774 | 23827896 |
| chr21 | 24006708 | 24006836 |
| chr21 | 24129079 | 24129211 |
| chr21 | 24299891 | 24300027 |
| chr21 | 24456953 | 24457077 |
| chr21 | 24530892 | 24531030 |
| chr21 | 24758728 | 24758880 |
| chr21 | 24923189 | 24923329 |
| chr21 | 24996413 | 24996559 |
| chr21 | 25199050 | 25199172 |
| chr21 | 25421164 | 25421306 |
| chr21 | 25476893 | 25477053 |
| chr21 | 25672403 | 25672537 |
| chr21 | 25795150 | 25795272 |
| chr21 | 25940993 | 25941141 |
| chr21 | 26163306 | 26163444 |
| chr21 | 26314113 | 26314259 |
| chr21 | 26456269 | 26456399 |
| chr21 | 26525118 | 26525264 |
| chr21 | 26698639 | 26698783 |
| chr21 | 26835737 | 26835875 |
| chr21 | 27052787 | 27052943 |
| chr21 | 27115709 | 27115835 |
| chr21 | 27316471 | 27316598 |
| chr21 | 27526313 | 27526437 |
| chr21 | 27543845 | 27544166 |
| chr21 | 27767617 | 27767741 |
| chr21 | 27937495 | 27937617 |
| chr21 | 28033945 | 28034069 |
| chr21 | 28219227 | 28219355 |
| chr21 | 28291806 | 28291960 |
| chr21 | 28477668 | 28477790 |
| chr21 | 28725256 | 28725402 |
| chr21 | 28829301 | 28829441 |
| chr21 | 29003120 | 29003260 |
| chr21 | 29111983 | 29112111 |
| chr21 | 29205622 | 29205746 |
| chr21 | 29442798 | 29442928 |
| chr21 | 29597903 | 29598029 |
| chr21 | 29706080 | 29706220 |
| chr21 | 29782703 | 29782869 |
| chr21 | 30066482 | 30066626 |
| chr21 | 30201029 | 30201169 |
| chr21 | 30241904 | 30242032 |
| chr21 | 30476776 | 30476900 |
| chr21 | 30605799 | 30605923 |
| chr21 | 30685529 | 30685665 |
| chr21 | 30849554 | 30849679 |
| chr21 | 30981189 | 30981321 |
| chr21 | 31208151 | 31208275 |
| chr21 | 31347448 | 31347582 |
| chr21 | 31542121 | 31542245 |
| chr21 | 31646767 | 31646891 |
| chr21 | 31728700 | 31728828 |
| chr21 | 31899706 | 31899846 |
| chr21 | 32058588 | 32058730 |
| chr21 | 32258517 | 32258645 |
| chr21 | 32452872 | 32453020 |
| chr21 | 32605294 | 32605428 |
| chr21 | 32636018 | 32636160 |
| chr21 | 32895005 | 32895141 |
| chr21 | 33002166 | 33002294 |
| chr21 | 33171623 | 33171745 |
| chr21 | 33325961 | 33326085 |
| chr21 | 33421808 | 33421946 |
| chr21 | 33553065 | 33553187 |
| chr21 | 33789771 | 33789909 |
| chr21 | 33801129 | 33801253 |
| chr21 | 33938019 | 33938147 |
| chr21 | 34047428 | 34047564 |
| chr21 | 34220532 | 34220688 |
| chr21 | 34326895 | 34327033 |
| chr21 | 34511796 | 34511926 |
| chr21 | 34697921 | 34698057 |
| chr21 | 34752736 | 34752870 |
| chr21 | 34832080 | 34832206 |
| chr21 | 34876992 | 34877124 |
| chr21 | 34926184 | 34926308 |
| chr21 | 34975684 | 34975966 |
| chr21 | 35093161 | 35093297 |
| chr21 | 35303924 | 35304048 |
| chr21 | 35403703 | 35403863 |
| chr21 | 35520729 | 35520885 |
| chr21 | 35673484 | 35673610 |
| chr21 | 35795336 | 35795458 |
| chr21 | 35953513 | 35953637 |
| chr21 | 36158345 | 36158750 |
| chr21 | 36254462 | 36254779 |
| chr21 | 36383548 | 36383676 |
| chr21 | 36647525 | 36647655 |
| chr21 | 36789489 | 36789631 |
| chr21 | 36965437 | 36965561 |
| chr21 | 37073080 | 37073420 |
| chr21 | 37169503 | 37169850 |
| chr21 | 37340921 | 37341045 |
| chr21 | 37530467 | 37530607 |
| chr21 | 37695395 | 37695543 |
| chr21 | 37838381 | 37838503 |
| chr21 | 37962133 | 37962259 |
| chr21 | 38061588 | 38061710 |
| chr21 | 38219110 | 38219236 |
| chr21 | 38358191 | 38358353 |
| chr21 | 38521786 | 38521918 |
| chr21 | 38774705 | 38774833 |
| chr21 | 38816272 | 38816418 |
| chr21 | 38991261 | 38991395 |
| chr21 | 39145979 | 39146103 |
| chr21 | 39293695 | 39293835 |
| chr21 | 39440882 | 39441004 |
| chr21 | 39610994 | 39611124 |
| chr21 | 39698003 | 39698131 |
| chr21 | 39955916 | 39956042 |
| chr21 | 40019065 | 40019217 |
| chr21 | 40141782 | 40141914 |
| chr21 | 40338747 | 40338905 |
| chr21 | 40466161 | 40466287 |
| chr21 | 40715453 | 40715591 |
| chr21 | 40769048 | 40769196 |
| chr21 | 41005816 | 41005956 |
| chr21 | 41172390 | 41172534 |
| chr21 | 41326537 | 41326665 |
| chr21 | 41363690 | 41363816 |
| chr21 | 41601772 | 41601898 |
| chr21 | 41644846 | 41644970 |
| chr21 | 41806738 | 41806866 |
| chr21 | 41959579 | 41959701 |
| chr21 | 42115228 | 42115376 |
| chr21 | 42278578 | 42278716 |
| chr21 | 42485724 | 42485858 |
| chr21 | 42647780 | 42647904 |
| chr21 | 42729324 | 42729446 |
| chr21 | 42836559 | 42836683 |
| chr21 | 43095366 | 43095490 |
| chr21 | 43130251 | 43130383 |
| chr21 | 43330508 | 43330662 |
| chr21 | 43495741 | 43495865 |
| chr21 | 43627631 | 43627755 |
| chr21 | 43813411 | 43813539 |
| chr21 | 44011315 | 44011439 |
| chr21 | 44152561 | 44152685 |
| chr21 | 44201725 | 44201847 |
| chr21 | 44401084 | 44401212 |
| chr21 | 44549588 | 44549728 |
| chr21 | 44696415 | 44696536 |
| chr21 | 44821823 | 44821947 |
| chr21 | 45014239 | 45014365 |
| chr21 | 45123571 | 45123697 |
| chr21 | 45259745 | 45259871 |
| chr21 | 45457264 | 45457506 |
| chr21 | 45639282 | 45639408 |
| chr21 | 45827572 | 45827696 |
| chr21 | 45955312 | 45955444 |
| chr21 | 46118161 | 46118285 |
| chr21 | 46143346 | 46143470 |
| chr21 | 46389698 | 46389834 |
| chr21 | 46525898 | 46526026 |
| chr21 | 46608549 | 46608691 |
| chr21 | 46816277 | 46816421 |
| chr21 | 46919878 | 46920004 |
| chr21 | 47155258 | 47155404 |
| chr21 | 47224309 | 47224451 |
| chr21 | 47429733 | 47429857 |
| chr21 | 47516313 | 47516439 |
| chr21 | 47709059 | 47709183 |
| chr21 | 47881225 | 47881363 |
| chr21 | 48050314 | 48050436 |
| chr22 | 16238522 | 16238648 |
| chr22 | 16455113 | 16455287 |
| chr22 | 16630109 | 16630231 |
| chr22 | 17020094 | 17020216 |
| chr22 | 17027632 | 17027788 |
| chr22 | 17253851 | 17253977 |
| chr22 | 17384068 | 17384192 |
| chr22 | 17548934 | 17549058 |
| chr22 | 17594831 | 17594993 |
| chr22 | 17828000 | 17828126 |
| chr22 | 17910528 | 17910656 |
| chr22 | 18089531 | 18089671 |
| chr22 | 18181787 | 18181927 |
| chr22 | 18448077 | 18448207 |
| chr22 | 18545297 | 18545423 |
| chr22 | 18641529 | 18641702 |
| chr22 | 18680788 | 18680916 |
| chr22 | 18682867 | 18682993 |
| chr22 | 18723059 | 18723185 |
| chr22 | 18725136 | 18725264 |
| chr22 | 18782473 | 18782605 |
| chr22 | 18847563 | 18847697 |
| chr22 | 18872790 | 18872918 |
| chr22 | 18894530 | 18894684 |
| chr22 | 19032137 | 19032271 |
| chr22 | 19098201 | 19098337 |
| chr22 | 19337846 | 19338181 |
| chr22 | 19433861 | 19433982 |
| chr22 | 19567704 | 19567846 |
| chr22 | 19771079 | 19771200 |
| chr22 | 19889735 | 19889875 |
| chr22 | 20045333 | 20045461 |
| chr22 | 20115474 | 20115600 |
| chr22 | 20283186 | 20283316 |
| chr22 | 20328034 | 20328160 |
| chr22 | 20330111 | 20330239 |
| chr22 | 20652620 | 20652748 |
| chr22 | 20654699 | 20654825 |
| chr22 | 20694793 | 20694919 |
| chr22 | 20696870 | 20696998 |
| chr22 | 20788986 | 20789124 |
| chr22 | 20908911 | 20909047 |
| chr22 | 21028102 | 21028226 |
| chr22 | 21285713 | 21285914 |
| chr22 | 21307016 | 21307140 |
| chr22 | 21468674 | 21468808 |
| chr22 | 21523749 | 21523875 |
| chr22 | 21525826 | 21525954 |
| chr22 | 21583596 | 21583726 |
| chr22 | 21650284 | 21650418 |
| chr22 | 21675423 | 21675551 |
| chr22 | 21677502 | 21677628 |
| chr22 | 21798855 | 21798977 |
| chr22 | 21958986 | 21959108 |
| chr22 | 22163381 | 22163511 |
| chr22 | 22214666 | 22214800 |
| chr22 | 22394153 | 22394289 |
| chr22 | 22540204 | 22540326 |
| chr22 | 22621429 | 22621585 |
| chr22 | 22802013 | 22802270 |
| chr22 | 22918509 | 22918653 |
| chr22 | 23022468 | 23022590 |
| chr22 | 23251806 | 23251930 |
| chr22 | 23354303 | 23354577 |
| chr22 | 23538305 | 23538437 |
| chr22 | 23588123 | 23588265 |
| chr22 | 23804263 | 23804387 |
| chr22 | 23945281 | 23945455 |
| chr22 | 24148211 | 24148357 |
| chr22 | 24255258 | 24255382 |
| chr22 | 24405127 | 24405263 |
| chr22 | 24500806 | 24500950 |
| chr22 | 24667521 | 24667790 |
| chr22 | 24846337 | 24846479 |
| chr22 | 24931434 | 24931564 |
| chr22 | 25177107 | 25177255 |
| chr22 | 25318002 | 25318124 |
| chr22 | 25373901 | 25374029 |
| chr22 | 25611815 | 25611939 |
| chr22 | 25725996 | 25726118 |
| chr22 | 25845931 | 25846055 |
| chr22 | 25973493 | 25973640 |
| chr22 | 26155738 | 26155870 |
| chr22 | 26319981 | 26320103 |
| chr22 | 26448145 | 26448267 |
| chr22 | 26555381 | 26555626 |
| chr22 | 26797068 | 26797190 |
| chr22 | 26968133 | 26968293 |
| chr22 | 27120397 | 27120519 |
| chr22 | 27200852 | 27201002 |
| chr22 | 27304457 | 27304579 |
| chr22 | 27590250 | 27590396 |
| chr22 | 27640691 | 27640817 |
| chr22 | 27857082 | 27857220 |
| chr22 | 27941652 | 27941784 |
| chr22 | 28090320 | 28090462 |
| chr22 | 28349747 | 28349885 |
| chr22 | 28488017 | 28488153 |
| chr22 | 28631832 | 28631972 |
| chr22 | 28775915 | 28776043 |
| chr22 | 28882927 | 28883061 |
| chr22 | 28964132 | 28964262 |
| chr22 | 29239730 | 29239852 |
| chr22 | 29369348 | 29369472 |
| chr22 | 29488100 | 29488244 |
| chr22 | 29568690 | 29568822 |
| chr22 | 29723833 | 29723955 |
| chr22 | 29854091 | 29854215 |
| chr22 | 30064679 | 30064801 |
| chr22 | 30300500 | 30300636 |
| chr22 | 30421905 | 30422029 |
| chr22 | 30597768 | 30597890 |
| chr22 | 30669913 | 30670043 |
| chr22 | 30755084 | 30755206 |
| chr22 | 31017253 | 31017431 |
| chr22 | 31138805 | 31138957 |
| chr22 | 31255976 | 31256114 |
| chr22 | 31369234 | 31369378 |
| chr22 | 31630806 | 31630946 |
| chr22 | 31698786 | 31698908 |
| chr22 | 31897489 | 31897633 |
| chr22 | 31977816 | 31977944 |
| chr22 | 32130592 | 32130716 |
| chr22 | 32339159 | 32339295 |
| chr22 | 32472298 | 32472575 |
| chr22 | 32637058 | 32637180 |
| chr22 | 32839672 | 32839830 |
| chr22 | 32985599 | 32985759 |
| chr22 | 33094301 | 33094423 |
| chr22 | 33291686 | 33291828 |
| chr22 | 33422142 | 33422268 |
| chr22 | 33507795 | 33507921 |
| chr22 | 33638344 | 33638470 |
| chr22 | 33820198 | 33820332 |
| chr22 | 33904692 | 33904836 |
| chr22 | 34086356 | 34086486 |
| chr22 | 34270617 | 34270745 |
| chr22 | 34408156 | 34408290 |
| chr22 | 34614840 | 34614968 |
| chr22 | 34691477 | 34691599 |
| chr22 | 34930401 | 34930525 |
| chr22 | 35100400 | 35100542 |
| chr22 | 35251854 | 35251998 |
| chr22 | 35332103 | 35332243 |
| chr22 | 35545247 | 35545371 |
| chr22 | 35676097 | 35676221 |
| chr22 | 35763532 | 35763666 |
| chr22 | 35899308 | 35899432 |
| chr22 | 36012800 | 36012934 |
| chr22 | 36155149 | 36155413 |
| chr22 | 36382378 | 36382502 |
| chr22 | 36457593 | 36457727 |
| chr22 | 36715943 | 36716079 |
| chr22 | 36844771 | 36844901 |
| chr22 | 36957818 | 36957944 |
| chr22 | 37176109 | 37176233 |
| chr22 | 37299455 | 37299577 |
| chr22 | 37386589 | 37386719 |
| chr22 | 37619702 | 37619836 |
| chr22 | 37761904 | 37762046 |
| chr22 | 37931693 | 37931825 |
| chr22 | 37992819 | 37992941 |
| chr22 | 38117859 | 38117983 |
| chr22 | 38300893 | 38301055 |
| chr22 | 38409516 | 38409646 |
| chr22 | 38683573 | 38683707 |
| chr22 | 38799791 | 38799947 |
| chr22 | 38925645 | 38925779 |
| chr22 | 39052959 | 39053083 |
| chr22 | 39205661 | 39205787 |
| chr22 | 39338089 | 39338223 |
| chr22 | 39557078 | 39557228 |
| chr22 | 39615227 | 39615353 |
| chr22 | 39840138 | 39840438 |
| chr22 | 39996802 | 39996942 |
| chr22 | 40103846 | 40103968 |
| chr22 | 40343957 | 40344099 |
| chr22 | 40473273 | 40473527 |
| chr22 | 40615190 | 40615324 |
| chr22 | 40659197 | 40659339 |
| chr22 | 40858920 | 40859044 |
| chr22 | 41034117 | 41034243 |
| chr22 | 41208312 | 41208440 |
| chr22 | 41351701 | 41351974 |
| chr22 | 41514124 | 41514252 |
| chr22 | 41555544 | 41555680 |
| chr22 | 41757557 | 41757707 |
| chr22 | 41867287 | 41867425 |
| chr22 | 42012695 | 42012963 |
| chr22 | 42169183 | 42169319 |
| chr22 | 42340754 | 42340914 |
| chr22 | 42544692 | 42544846 |
| chr22 | 42614288 | 42614412 |
| chr22 | 42869415 | 42869539 |
| chr22 | 42917080 | 42917208 |
| chr22 | 43113944 | 43114070 |
| chr22 | 43326072 | 43326198 |
| chr22 | 43426893 | 43427015 |
| chr22 | 43587400 | 43587546 |
| chr22 | 43688612 | 43688746 |
| chr22 | 43856586 | 43856722 |
| chr22 | 44056171 | 44056295 |
| chr22 | 44205291 | 44205415 |
| chr22 | 44259707 | 44259833 |
| chr22 | 44530738 | 44530862 |
| chr22 | 44646575 | 44646711 |
| chr22 | 44831770 | 44831892 |
| chr22 | 44925025 | 44925169 |
| chr22 | 45150573 | 45150707 |
| chr22 | 45184709 | 45184831 |
| chr22 | 45429797 | 45429923 |
| chr22 | 45596416 | 45596560 |
| chr22 | 45737226 | 45737358 |
| chr22 | 45864844 | 45864968 |
| chr22 | 45972530 | 45972674 |
| chr22 | 46106229 | 46106381 |
| chr22 | 46235379 | 46235503 |
| chr22 | 46428903 | 46429055 |
| chr22 | 46622836 | 46622976 |
| chr22 | 46770134 | 46770262 |
| chr22 | 46943743 | 46943869 |
| chr22 | 47045482 | 47045610 |
| chr22 | 47178537 | 47178659 |
| chr22 | 47325920 | 47326046 |
| chr22 | 47491101 | 47491231 |
| chr22 | 47644768 | 47644896 |
| chr22 | 47826244 | 47826384 |
| chr22 | 47915811 | 47915963 |
| chr22 | 48106653 | 48106777 |
| chr22 | 48270918 | 48271044 |
| chr22 | 48421269 | 48421415 |
| chr22 | 48501934 | 48502064 |
| chr22 | 48655852 | 48655980 |
| chr22 | 48832887 | 48833019 |
| chr22 | 48966259 | 48966383 |
| chr22 | 49171468 | 49171596 |
| chr22 | 49254422 | 49254560 |
| chr22 | 49481646 | 49481790 |
| chr22 | 49613300 | 49613424 |
| chr22 | 49803517 | 49803671 |
| chr22 | 49913148 | 49913272 |
| chr22 | 49990412 | 49990540 |
| chr22 | 50227653 | 50227791 |
| chr22 | 50282148 | 50282284 |
| chr22 | 50415125 | 50415246 |
| chr22 | 50595752 | 50595878 |
| chr22 | 50816529 | 50816683 |
| chr22 | 50891697 | 50891831 |
| chr22 | 51071848 | 51072022 |
| chr22 | 51175710 | 51176309 |
| chr22 | 51226286 | 51226416 |
| chrX | 2711190 | 2711431 |
| chrX | 2744729 | 2744853 |
| chrX | 2783511 | 2783635 |
| chrX | 2814108 | 2814232 |
| chrX | 2865244 | 2865370 |
| chrX | 2917026 | 2917147 |
| chrX | 2997079 | 2997203 |
| chrX | 3031630 | 3031752 |
| chrX | 3106625 | 3106765 |
| chrX | 3153384 | 3153522 |
| chrX | 3220593 | 3220737 |
| chrX | 3232022 | 3232146 |
| chrX | 3292959 | 3293099 |
| chrX | 3518288 | 3518412 |
| chrX | 3646327 | 3646469 |
| chrX | 3858109 | 3858261 |
| chrX | 4093576 | 4093700 |
| chrX | 4167195 | 4167319 |
| chrX | 4325077 | 4325201 |
| chrX | 4502845 | 4502967 |
| chrX | 4712250 | 4712406 |
| chrX | 4964876 | 4965030 |
| chrX | 5119702 | 5119838 |
| chrX | 5419131 | 5419271 |
| chrX | 5680461 | 5680603 |
| chrX | 5828432 | 5829002 |
| chrX | 5912813 | 5912963 |
| chrX | 6070996 | 6071126 |
| chrX | 6236986 | 6237130 |
| chrX | 6295237 | 6295375 |
| chrX | 6322530 | 6322719 |
| chrX | 6379769 | 6379907 |
| chrX | 6432834 | 6432955 |
| chrX | 6522339 | 6522586 |
| chrX | 6596559 | 6596695 |
| chrX | 6685392 | 6685516 |
| chrX | 6899387 | 6899525 |
| chrX | 7000378 | 7000504 |
| chrX | 7138014 | 7138154 |
| chrX | 7262867 | 7263190 |
| chrX | 7318893 | 7319021 |
| chrX | 7357975 | 7358121 |
| chrX | 7433814 | 7433938 |
| chrX | 7458522 | 7458644 |
| chrX | 7483012 | 7483154 |
| chrX | 7556747 | 7557004 |
| chrX | 7615585 | 7615706 |
| chrX | 7675674 | 7675802 |
| chrX | 7741639 | 7741767 |
| chrX | 7806249 | 7806391 |
| chrX | 7850681 | 7850805 |
| chrX | 7929414 | 7929540 |
| chrX | 7997997 | 7998275 |
| chrX | 8053166 | 8053294 |
| chrX | 8091836 | 8091960 |
| chrX | 8114035 | 8114173 |
| chrX | 8178614 | 8178738 |
| chrX | 8245630 | 8245754 |
| chrX | 8313259 | 8313403 |
| chrX | 8447999 | 8448135 |
| chrX | 8710956 | 8711094 |
| chrX | 8784205 | 8784329 |
| chrX | 8970269 | 8970391 |
| chrX | 9247963 | 9248087 |
| chrX | 9466273 | 9466397 |
| chrX | 9584490 | 9584620 |
| chrX | 9678519 | 9678649 |
| chrX | 9882488 | 9882620 |
| chrX | 9948752 | 9948910 |
| chrX | 10125326 | 10125456 |
| chrX | 10247838 | 10247960 |
| chrX | 10471953 | 10472219 |
| chrX | 10666930 | 10667131 |
| chrX | 10836368 | 10836494 |
| chrX | 10849740 | 10850235 |
| chrX | 11137096 | 11137217 |
| chrX | 11264630 | 11264828 |
| chrX | 11401249 | 11401436 |
| chrX | 11476136 | 11476264 |
| chrX | 11744044 | 11744172 |
| chrX | 11788294 | 11788426 |
| chrX | 12024826 | 12024966 |
| chrX | 12113666 | 12113816 |
| chrX | 12277724 | 12277864 |
| chrX | 12539229 | 12539361 |
| chrX | 12671084 | 12671210 |
| chrX | 12909524 | 12909652 |
| chrX | 13018325 | 13018447 |
| chrX | 13191256 | 13191382 |
| chrX | 13308115 | 13308239 |
| chrX | 13498220 | 13498344 |
| chrX | 13551546 | 13551670 |
| chrX | 13772960 | 13773082 |
| chrX | 13917757 | 13917897 |
| chrX | 14149393 | 14149535 |
| chrX | 14429759 | 14430037 |
| chrX | 14509443 | 14509583 |
| chrX | 14634822 | 14634944 |
| chrX | 14884569 | 14884693 |
| chrX | 14962312 | 14962452 |
| chrX | 15094871 | 15095017 |
| chrX | 15263673 | 15263813 |
| chrX | 15359360 | 15359486 |
| chrX | 15542034 | 15542164 |
| chrX | 15651187 | 15651309 |
| chrX | 15909647 | 15909783 |
| chrX | 16156399 | 16156537 |
| chrX | 16222963 | 16223085 |
| chrX | 16275058 | 16275210 |
| chrX | 16351668 | 16351792 |
| chrX | 16393516 | 16393646 |
| chrX | 16434416 | 16434540 |
| chrX | 16453018 | 16453158 |
| chrX | 16511634 | 16511768 |
| chrX | 16525773 | 16525907 |
| chrX | 16558368 | 16558502 |
| chrX | 16584789 | 16584945 |
| chrX | 16624152 | 16624425 |
| chrX | 16680175 | 16680438 |
| chrX | 16789968 | 16790090 |
| chrX | 17011075 | 17011340 |
| chrX | 17232693 | 17232963 |
| chrX | 17406979 | 17407228 |
| chrX | 17475944 | 17476065 |
| chrX | 17650506 | 17650627 |
| chrX | 17847412 | 17847675 |
| chrX | 17957098 | 17957358 |
| chrX | 18120121 | 18120383 |
| chrX | 18197641 | 18197779 |
| chrX | 18369523 | 18369645 |
| chrX | 18590676 | 18591254 |
| chrX | 18653546 | 18653671 |
| chrX | 18921271 | 18921494 |
| chrX | 19215217 | 19215341 |
| chrX | 19353135 | 19353462 |
| chrX | 19447070 | 19447319 |
| chrX | 19646490 | 19646614 |
| chrX | 19716678 | 19716824 |
| chrX | 19800019 | 19800283 |
| chrX | 19828475 | 19828735 |
| chrX | 19883284 | 19883546 |
| chrX | 19911851 | 19912115 |
| chrX | 19965648 | 19965924 |
| chrX | 20004452 | 20004599 |
| chrX | 20039846 | 20039970 |
| chrX | 20130810 | 20131085 |
| chrX | 20198599 | 20198720 |
| chrX | 20378566 | 20378694 |
| chrX | 20603773 | 20603895 |
| chrX | 20762284 | 20762436 |
| chrX | 20950209 | 20950331 |
| chrX | 21174150 | 21174424 |
| chrX | 21260996 | 21261117 |
| chrX | 21426360 | 21426643 |
| chrX | 21593635 | 21593897 |
| chrX | 21742254 | 21742378 |
| chrX | 21854529 | 21854679 |
| chrX | 22083824 | 22084091 |
| chrX | 22104589 | 22104753 |
| chrX | 22345578 | 22345724 |
| chrX | 22479309 | 22479465 |
| chrX | 22568620 | 22568744 |
| chrX | 22808040 | 22808162 |
| chrX | 22895389 | 22895539 |
| chrX | 23039567 | 23039699 |
| chrX | 23288665 | 23288813 |
| chrX | 23436427 | 23436551 |
| chrX | 23566477 | 23566619 |
| chrX | 23637048 | 23637172 |
| chrX | 23815946 | 23816086 |
| chrX | 23904594 | 23904718 |
| chrX | 24179366 | 24179490 |
| chrX | 24339589 | 24339868 |
| chrX | 24386506 | 24386636 |
| chrX | 24558207 | 24558347 |
| chrX | 24788183 | 24788315 |
| chrX | 25085257 | 25085383 |
| chrX | 25217057 | 25217181 |
| chrX | 25375393 | 25375515 |
| chrX | 25534543 | 25534667 |
| chrX | 25720038 | 25720162 |
| chrX | 25848159 | 25848289 |
| chrX | 25890788 | 25890944 |
| chrX | 25977863 | 25977985 |
| chrX | 26017442 | 26017566 |
| chrX | 26079674 | 26079812 |
| chrX | 26124738 | 26124884 |
| chrX | 26168189 | 26168315 |
| chrX | 26224387 | 26224543 |
| chrX | 26366115 | 26366253 |
| chrX | 26513051 | 26513195 |
| chrX | 26604040 | 26604166 |
| chrX | 26768416 | 26768576 |
| chrX | 26933552 | 26933678 |
| chrX | 27189327 | 27189451 |
| chrX | 27391161 | 27391311 |
| chrX | 27689752 | 27689876 |
| chrX | 27933326 | 27933584 |
| chrX | 27985638 | 27985762 |
| chrX | 28041338 | 28041464 |
| chrX | 28067856 | 28067996 |
| chrX | 28135426 | 28135552 |
| chrX | 28152416 | 28152540 |
| chrX | 28241576 | 28241718 |
| chrX | 28393616 | 28393756 |
| chrX | 28555021 | 28555167 |
| chrX | 28793327 | 28793465 |
| chrX | 28872556 | 28872678 |
| chrX | 29007021 | 29007161 |
| chrX | 29269304 | 29269426 |
| chrX | 29329956 | 29330084 |
| chrX | 29503544 | 29503672 |
| chrX | 29707699 | 29707831 |
| chrX | 29864248 | 29864564 |
| chrX | 30011250 | 30011493 |
| chrX | 30138679 | 30138831 |
| chrX | 30325524 | 30325889 |
| chrX | 30498120 | 30498399 |
| chrX | 30699077 | 30699201 |
| chrX | 30944147 | 30944297 |
| chrX | 31088817 | 31088941 |
| chrX | 31183543 | 31183667 |
| chrX | 31390137 | 31390267 |
| chrX | 31413138 | 31413258 |
| chrX | 31684279 | 31684419 |
| chrX | 31836870 | 31836998 |
| chrX | 31923993 | 31924117 |
| chrX | 32127975 | 32128139 |
| chrX | 32278017 | 32278155 |
| chrX | 32356767 | 32356893 |
| chrX | 32586068 | 32586212 |
| chrX | 32659855 | 32659983 |
| chrX | 32757728 | 32757850 |
| chrX | 32985071 | 32985211 |
| chrX | 33104673 | 33104811 |
| chrX | 33224664 | 33224790 |
| chrX | 33347752 | 33347880 |
| chrX | 33610999 | 33611133 |
| chrX | 33691990 | 33692114 |
| chrX | 33940986 | 33941112 |
| chrX | 34219581 | 34219715 |
| chrX | 34390986 | 34391130 |
| chrX | 34506944 | 34507076 |
| chrX | 34576395 | 34576527 |
| chrX | 34627991 | 34628121 |
| chrX | 34637772 | 34637910 |
| chrX | 34681602 | 34681726 |
| chrX | 34740668 | 34740790 |
| chrX | 34809738 | 34809880 |
| chrX | 34850764 | 34850906 |
| chrX | 34891226 | 34891352 |
| chrX | 34986886 | 34987016 |
| chrX | 35109512 | 35109640 |
| chrX | 35225895 | 35226033 |
| chrX | 35336268 | 35336412 |
| chrX | 35498616 | 35498888 |
| chrX | 35708143 | 35708289 |
| chrX | 35762284 | 35762428 |
| chrX | 35908888 | 35909030 |
| chrX | 36074759 | 36075025 |
| chrX | 36270956 | 36271080 |
| chrX | 36491807 | 36491939 |
| chrX | 36613668 | 36613806 |
| chrX | 36700017 | 36700157 |
| chrX | 36978886 | 36979008 |
| chrX | 37176268 | 37176546 |
| chrX | 37369393 | 37369536 |
| chrX | 37504712 | 37504998 |
| chrX | 37659287 | 37659557 |
| chrX | 37792694 | 37792973 |
| chrX | 37885010 | 37885142 |
| chrX | 38107281 | 38107427 |
| chrX | 38256735 | 38256887 |
| chrX | 38353826 | 38353948 |
| chrX | 38472788 | 38473128 |
| chrX | 38737289 | 38737431 |
| chrX | 38789181 | 38789303 |
| chrX | 38975690 | 38975830 |
| chrX | 39096616 | 39096738 |
| chrX | 39321339 | 39321507 |
| chrX | 39423131 | 39423279 |
| chrX | 39516765 | 39516907 |
| chrX | 39731085 | 39731227 |
| chrX | 39907923 | 39908169 |
| chrX | 40037057 | 40037182 |
| chrX | 40159977 | 40160121 |
| chrX | 40453730 | 40453927 |
| chrX | 40605783 | 40605905 |
| chrX | 40699377 | 40699624 |
| chrX | 40903611 | 40903745 |
| chrX | 41041149 | 41041297 |
| chrX | 41278493 | 41278615 |
| chrX | 41418131 | 41418281 |
| chrX | 41459642 | 41459780 |
| chrX | 41707333 | 41707573 |
| chrX | 41764539 | 41764759 |
| chrX | 42026848 | 42026970 |
| chrX | 42105281 | 42105425 |
| chrX | 42253959 | 42254117 |
| chrX | 42397645 | 42397765 |
| chrX | 42615537 | 42615659 |
| chrX | 42702753 | 42702887 |
| chrX | 42942704 | 42942832 |
| chrX | 42979695 | 42979831 |
| chrX | 43134805 | 43134927 |
| chrX | 43377690 | 43377828 |
| chrX | 43525614 | 43525734 |
| chrX | 43591410 | 43591550 |
| chrX | 43832296 | 43832422 |
| chrX | 43877429 | 43877557 |
| chrX | 44084279 | 44084401 |
| chrX | 44216401 | 44216537 |
| chrX | 44296328 | 44296452 |
| chrX | 44514431 | 44514565 |
| chrX | 44565856 | 44565982 |
| chrX | 44599649 | 44599771 |
| chrX | 44664787 | 44664923 |
| chrX | 44730915 | 44731045 |
| chrX | 44783669 | 44783824 |
| chrX | 44819453 | 44819589 |
| chrX | 44833269 | 44833391 |
| chrX | 44840018 | 44840139 |
| chrX | 44880347 | 44880477 |
| chrX | 44947424 | 44947545 |
| chrX | 45114532 | 45114674 |
| chrX | 45307495 | 45307619 |
| chrX | 45482792 | 45482932 |
| chrX | 45555497 | 45555621 |
| chrX | 45640441 | 45640595 |
| chrX | 45680334 | 45680474 |
| chrX | 45789637 | 45789761 |
| chrX | 45848009 | 45848260 |
| chrX | 46053330 | 46053482 |
| chrX | 46127254 | 46127394 |
| chrX | 46316538 | 46316662 |
| chrX | 46464912 | 46465056 |
| chrX | 46559851 | 46559983 |
| chrX | 46708486 | 46708618 |
| chrX | 46759572 | 46759720 |
| chrX | 46773887 | 46774031 |
| chrX | 46843207 | 46843331 |
| chrX | 46853989 | 46854113 |
| chrX | 46866078 | 46866200 |
| chrX | 46881482 | 46881620 |
| chrX | 46928221 | 46928351 |
| chrX | 46960601 | 46960739 |
| chrX | 47036525 | 47036671 |
| chrX | 47060792 | 47060916 |
| chrX | 47123316 | 47123462 |
| chrX | 47231270 | 47231394 |
| chrX | 47306334 | 47306458 |
| chrX | 47500209 | 47500335 |
| chrX | 47720765 | 47720889 |
| chrX | 47745784 | 47745906 |
| chrX | 48016555 | 48016681 |
| chrX | 48137441 | 48137565 |
| chrX | 48323838 | 48324231 |
| chrX | 48374764 | 48375012 |
| chrX | 48612903 | 48613029 |
| chrX | 48744604 | 48744795 |
| chrX | 48936735 | 48936883 |
| chrX | 48991083 | 48991208 |
| chrX | 49131266 | 49131390 |
| chrX | 49373468 | 49373735 |
| chrX | 49477275 | 49477397 |
| chrX | 49570400 | 49570606 |
| chrX | 49713645 | 49713766 |
| chrX | 49964149 | 49964271 |
| chrX | 50068461 | 50068635 |
| chrX | 50259388 | 50259509 |
| chrX | 50429764 | 50430029 |
| chrX | 50454125 | 50454370 |
| chrX | 50495655 | 50496218 |
| chrX | 50513599 | 50513727 |
| chrX | 50516808 | 50517360 |
| chrX | 50529029 | 50529207 |
| chrX | 50540659 | 50540932 |
| chrX | 50549389 | 50549535 |
| chrX | 50555727 | 50555994 |
| chrX | 50582650 | 50582808 |
| chrX | 50658949 | 50659070 |
| chrX | 50677612 | 50677820 |
| chrX | 50737473 | 50737652 |
| chrX | 50817418 | 50817552 |
| chrX | 50866453 | 50866697 |
| chrX | 50931934 | 50932072 |
| chrX | 51084103 | 51084255 |
| chrX | 51317845 | 51317977 |
| chrX | 51405441 | 51405579 |
| chrX | 51581163 | 51581287 |
| chrX | 51746642 | 51746826 |
| chrX | 51896234 | 51896510 |
| chrX | 51976633 | 51976754 |
| chrX | 52060794 | 52061063 |
| chrX | 52081010 | 52081131 |
| chrX | 52096832 | 52096959 |
| chrX | 52585552 | 52585690 |
| chrX | 52637344 | 52637496 |
| chrX | 52662240 | 52662378 |
| chrX | 52854262 | 52854388 |
| chrX | 52858604 | 52858725 |
| chrX | 52890544 | 52890665 |
| chrX | 52952530 | 52952727 |
| chrX | 53009099 | 53009316 |
| chrX | 53256051 | 53256172 |
| chrX | 53406149 | 53406569 |
| chrX | 53567140 | 53567262 |
| chrX | 53717801 | 53718073 |
| chrX | 53858525 | 53858721 |
| chrX | 54023427 | 54023549 |
| chrX | 54064783 | 54065259 |
| chrX | 54152682 | 54152862 |
| chrX | 54185726 | 54185846 |
| chrX | 54198393 | 54198513 |
| chrX | 54214001 | 54214124 |
| chrX | 54275131 | 54275383 |
| chrX | 54327942 | 54328063 |
| chrX | 54348581 | 54348701 |
| chrX | 54414600 | 54414856 |
| chrX | 54438601 | 54438780 |
| chrX | 54488273 | 54488402 |
| chrX | 54506400 | 54506867 |
| chrX | 54730754 | 54730892 |
| chrX | 54853760 | 54853906 |
| chrX | 55045092 | 55045222 |
| chrX | 55232646 | 55232774 |
| chrX | 55390302 | 55390424 |
| chrX | 55510416 | 55510546 |
| chrX | 55694446 | 55694594 |
| chrX | 55820943 | 55821069 |
| chrX | 55879514 | 55879638 |
| chrX | 56107858 | 56107988 |
| chrX | 56292858 | 56292996 |
| chrX | 56317987 | 56318259 |
| chrX | 56487688 | 56487969 |
| chrX | 56558720 | 56558886 |
| chrX | 56585338 | 56585496 |
| chrX | 56615921 | 56616045 |
| chrX | 56680845 | 56681097 |
| chrX | 56728568 | 56728716 |
| chrX | 56776911 | 56777067 |
| chrX | 56851911 | 56852051 |
| chrX | 56912284 | 56912420 |
| chrX | 56952073 | 56952199 |
| chrX | 57019774 | 57019918 |
| chrX | 57089063 | 57089237 |
| chrX | 57225294 | 57225418 |
| chrX | 57386438 | 57386576 |
| chrX | 57627940 | 57628116 |
| chrX | 57725587 | 57725772 |
| chrX | 57848753 | 57848874 |
| chrX | 58015499 | 58015620 |
| chrX | 58140721 | 58140897 |
| chrX | 58322400 | 58322521 |
| chrX | 61939269 | 61939391 |
| chrX | 62118298 | 62118424 |
| chrX | 62148670 | 62148853 |
| chrX | 62347076 | 62347197 |
| chrX | 62407205 | 62407457 |
| chrX | 62500205 | 62500329 |
| chrX | 62520580 | 62520701 |
| chrX | 62574297 | 62574574 |
| chrX | 62605975 | 62606152 |
| chrX | 62614758 | 62614878 |
| chrX | 62646918 | 62647039 |
| chrX | 62731218 | 62731338 |
| chrX | 62785579 | 62785722 |
| chrX | 62961018 | 62961289 |
| chrX | 63130583 | 63130844 |
| chrX | 63298299 | 63298423 |
| chrX | 63481438 | 63481695 |
| chrX | 63600199 | 63600327 |
| chrX | 63790192 | 63790443 |
| chrX | 64007979 | 64008253 |
| chrX | 64306267 | 64306454 |
| chrX | 64356533 | 64356654 |
| chrX | 64436685 | 64436819 |
| chrX | 64455618 | 64455775 |
| chrX | 64484806 | 64484982 |
| chrX | 64493410 | 64493531 |
| chrX | 64556611 | 64556732 |
| chrX | 64594938 | 64595063 |
| chrX | 64634357 | 64634545 |
| chrX | 64666736 | 64666996 |
| chrX | 64725026 | 64725275 |
| chrX | 64975843 | 64975963 |
| chrX | 65182252 | 65182390 |
| chrX | 65411489 | 65411615 |
| chrX | 65521470 | 65521608 |
| chrX | 65720138 | 65720284 |
| chrX | 65878333 | 65878593 |
| chrX | 65953879 | 65954068 |
| chrX | 65987458 | 65987722 |
| chrX | 66047050 | 66047171 |
| chrX | 66082232 | 66082355 |
| chrX | 66138074 | 66138217 |
| chrX | 66181882 | 66182025 |
| chrX | 66221839 | 66221960 |
| chrX | 66351387 | 66351514 |
| chrX | 66595954 | 66596150 |
| chrX | 66780305 | 66780426 |
| chrX | 66861373 | 66861493 |
| chrX | 66956800 | 66956928 |
| chrX | 67226803 | 67226929 |
| chrX | 67330141 | 67330495 |
| chrX | 67408462 | 67408594 |
| chrX | 67585140 | 67585634 |
| chrX | 67633237 | 67633460 |
| chrX | 67647410 | 67647552 |
| chrX | 67654250 | 67654820 |
| chrX | 67681596 | 67681740 |
| chrX | 67739811 | 67740067 |
| chrX | 67795450 | 67795584 |
| chrX | 67830018 | 67830139 |
| chrX | 67882150 | 67882399 |
| chrX | 67897824 | 67897968 |
| chrX | 67980769 | 67980911 |
| chrX | 68089902 | 68090028 |
| chrX | 68275432 | 68275552 |
| chrX | 68316037 | 68316158 |
| chrX | 68486846 | 68486972 |
| chrX | 68625731 | 68625855 |
| chrX | 68841041 | 68841177 |
| chrX | 68943661 | 68943831 |
| chrX | 69099074 | 69099644 |
| chrX | 69213405 | 69213660 |
| chrX | 69352708 | 69352840 |
| chrX | 69409840 | 69409980 |
| chrX | 69420956 | 69421080 |
| chrX | 69465236 | 69465358 |
| chrX | 69491727 | 69491979 |
| chrX | 69546470 | 69546598 |
| chrX | 69572471 | 69572719 |
| chrX | 69626166 | 69626436 |
| chrX | 69676656 | 69676780 |
| chrX | 69724641 | 69724895 |
| chrX | 69744193 | 69744317 |
| chrX | 69793727 | 69793869 |
| chrX | 69871518 | 69871654 |
| chrX | 69918421 | 69918545 |
| chrX | 69929777 | 69929907 |
| chrX | 69966644 | 69966786 |
| chrX | 70043906 | 70044046 |
| chrX | 70130382 | 70130503 |
| chrX | 70141853 | 70142092 |
| chrX | 70196008 | 70196130 |
| chrX | 70241669 | 70241791 |
| chrX | 70329141 | 70329470 |
| chrX | 70343896 | 70344016 |
| chrX | 70344017 | 70344137 |
| chrX | 70384759 | 70384880 |
| chrX | 70462476 | 70462596 |
| chrX | 70620725 | 70620855 |
| chrX | 70708402 | 70708523 |
| chrX | 70861270 | 70861391 |
| chrX | 71030047 | 71030185 |
| chrX | 71163732 | 71163886 |
| chrX | 71394034 | 71394184 |
| chrX | 71523538 | 71523662 |
| chrX | 71607359 | 71607497 |
| chrX | 71762066 | 71762207 |
| chrX | 71948064 | 71948213 |
| chrX | 72010233 | 72010379 |
| chrX | 72150111 | 72150257 |
| chrX | 72295142 | 72295302 |
| chrX | 72413086 | 72413209 |
| chrX | 72630770 | 72630896 |
| chrX | 72675608 | 72675740 |
| chrX | 72798751 | 72798872 |
| chrX | 72973383 | 72973511 |
| chrX | 73095887 | 73096278 |
| chrX | 73330330 | 73330480 |
| chrX | 73435977 | 73436256 |
| chrX | 73657534 | 73658094 |
| chrX | 73795270 | 73795422 |
| chrX | 73972201 | 73972322 |
| chrX | 74065843 | 74065965 |
| chrX | 74231766 | 74232034 |
| chrX | 74334601 | 74334737 |
| chrX | 74358913 | 74359037 |
| chrX | 74416813 | 74416942 |
| chrX | 74431638 | 74431759 |
| chrX | 74506560 | 74506682 |
| chrX | 74541258 | 74541504 |
| chrX | 74600351 | 74600667 |
| chrX | 74618186 | 74618310 |
| chrX | 74633591 | 74633801 |
| chrX | 74636044 | 74636475 |
| chrX | 74678390 | 74678664 |
| chrX | 74788056 | 74788179 |
| chrX | 74998214 | 74998475 |
| chrX | 75150085 | 75150229 |
| chrX | 75342921 | 75343049 |
| chrX | 75373262 | 75373410 |
| chrX | 75569634 | 75569758 |
| chrX | 75779362 | 75779484 |
| chrX | 76052156 | 76052278 |
| chrX | 76125662 | 76125935 |
| chrX | 76151841 | 76151962 |
| chrX | 76241422 | 76241542 |
| chrX | 76284926 | 76285047 |
| chrX | 76308908 | 76309040 |
| chrX | 76369319 | 76369441 |
| chrX | 76411214 | 76411340 |
| chrX | 76649706 | 76649831 |
| chrX | 76760406 | 76760530 |
| chrX | 76873770 | 76873892 |
| chrX | 77023156 | 77023310 |
| chrX | 77193246 | 77193370 |
| chrX | 77370466 | 77370691 |
| chrX | 77577561 | 77577685 |
| chrX | 77700307 | 77700451 |
| chrX | 77874774 | 77874969 |
| chrX | 77944314 | 77944444 |
| chrX | 77968120 | 77968248 |
| chrX | 78027971 | 78028113 |
| chrX | 78041481 | 78041613 |
| chrX | 78081596 | 78081738 |
| chrX | 78142029 | 78142150 |
| chrX | 78187371 | 78187494 |
| chrX | 78247273 | 78247397 |
| chrX | 78542729 | 78542869 |
| chrX | 78656930 | 78657052 |
| chrX | 78942645 | 78942771 |
| chrX | 79143071 | 79143203 |
| chrX | 79348544 | 79348678 |
| chrX | 79419389 | 79419513 |
| chrX | 79611474 | 79611595 |
| chrX | 79748098 | 79748220 |
| chrX | 79958639 | 79958763 |
| chrX | 80046653 | 80046777 |
| chrX | 80242520 | 80242678 |
| chrX | 80417047 | 80417177 |
| chrX | 80485998 | 80486187 |
| chrX | 80677080 | 80677220 |
| chrX | 80750395 | 80750519 |
| chrX | 80965178 | 80965312 |
| chrX | 81119455 | 81119607 |
| chrX | 81245149 | 81245281 |
| chrX | 81411880 | 81412022 |
| chrX | 81600411 | 81600569 |
| chrX | 81777507 | 81777633 |
| chrX | 81874560 | 81874682 |
| chrX | 81948605 | 81948737 |
| chrX | 82005816 | 82005966 |
| chrX | 82069476 | 82069600 |
| chrX | 82109030 | 82109154 |
| chrX | 82129510 | 82129634 |
| chrX | 82221903 | 82222029 |
| chrX | 82464528 | 82464650 |
| chrX | 82563437 | 82563565 |
| chrX | 82724899 | 82725077 |
| chrX | 82809928 | 82810052 |
| chrX | 82846792 | 82846932 |
| chrX | 82892450 | 82892578 |
| chrX | 82965762 | 82965952 |
| chrX | 82988202 | 82988334 |
| chrX | 82999903 | 83000041 |
| chrX | 83068162 | 83068323 |
| chrX | 83088173 | 83088296 |
| chrX | 83099733 | 83099861 |
| chrX | 83178802 | 83179048 |
| chrX | 83376573 | 83376699 |
| chrX | 83561337 | 83561489 |
| chrX | 83821403 | 83821539 |
| chrX | 84037675 | 84037807 |
| chrX | 84150117 | 84150245 |
| chrX | 84411620 | 84411764 |
| chrX | 84527833 | 84527967 |
| chrX | 84699588 | 84699800 |
| chrX | 84874340 | 84874466 |
| chrX | 85001887 | 85002025 |
| chrX | 85202898 | 85203085 |
| chrX | 85247559 | 85247725 |
| chrX | 85493895 | 85494017 |
| chrX | 85670033 | 85670167 |
| chrX | 85741943 | 85742071 |
| chrX | 86023413 | 86023547 |
| chrX | 86159088 | 86159212 |
| chrX | 86377880 | 86378012 |
| chrX | 86519359 | 86519483 |
| chrX | 86666352 | 86666482 |
| chrX | 86784095 | 86784225 |
| chrX | 87022481 | 87022623 |
| chrX | 87149558 | 87149690 |
| chrX | 87315072 | 87315196 |
| chrX | 87397976 | 87398100 |
| chrX | 87497695 | 87497843 |
| chrX | 87561477 | 87561619 |
| chrX | 87588721 | 87588877 |
| chrX | 87620323 | 87620457 |
| chrX | 87628556 | 87628692 |
| chrX | 87672900 | 87673026 |
| chrX | 87709448 | 87709572 |
| chrX | 87731429 | 87731575 |
| chrX | 87766724 | 87766848 |
| chrX | 87824821 | 87824961 |
| chrX | 87880175 | 87880343 |
| chrX | 87921905 | 87922026 |
| chrX | 88002567 | 88002768 |
| chrX | 88077458 | 88077608 |
| chrX | 88106705 | 88106855 |
| chrX | 88127124 | 88127252 |
| chrX | 88204677 | 88204799 |
| chrX | 88279901 | 88280049 |
| chrX | 88405347 | 88405471 |
| chrX | 88568443 | 88568567 |
| chrX | 88695989 | 88696113 |
| chrX | 88921909 | 88922045 |
| chrX | 89071404 | 89071542 |
| chrX | 89283709 | 89283835 |
| chrX | 89317752 | 89317908 |
| chrX | 89513919 | 89514045 |
| chrX | 89609005 | 89609157 |
| chrX | 89787519 | 89787647 |
| chrX | 89865787 | 89865931 |
| chrX | 90001083 | 90001219 |
| chrX | 90175715 | 90175873 |
| chrX | 90248665 | 90248789 |
| chrX | 90378600 | 90378728 |
| chrX | 90508475 | 90508597 |
| chrX | 90681225 | 90681353 |
| chrX | 90858920 | 90859042 |
| chrX | 91067964 | 91068086 |
| chrX | 91238364 | 91238504 |
| chrX | 91334080 | 91334202 |
| chrX | 91469204 | 91469332 |
| chrX | 91571680 | 91571802 |
| chrX | 91796932 | 91797082 |
| chrX | 91879482 | 91879604 |
| chrX | 92017468 | 92017608 |
| chrX | 92241284 | 92241408 |
| chrX | 92375840 | 92376012 |
| chrX | 92468862 | 92468992 |
| chrX | 92676672 | 92676812 |
| chrX | 92814525 | 92814649 |
| chrX | 93007842 | 93007966 |
| chrX | 93101268 | 93101396 |
| chrX | 93249021 | 93249141 |
| chrX | 93322800 | 93322928 |
| chrX | 93346894 | 93347016 |
| chrX | 93393441 | 93393565 |
| chrX | 93403562 | 93403696 |
| chrX | 93463695 | 93463823 |
| chrX | 93483635 | 93483759 |
| chrX | 93550195 | 93550319 |
| chrX | 93596047 | 93596171 |
| chrX | 93612172 | 93612294 |
| chrX | 93662953 | 93663077 |
| chrX | 93891343 | 93891467 |
| chrX | 93974593 | 93974713 |
| chrX | 94102714 | 94102860 |
| chrX | 94212353 | 94212505 |
| chrX | 94259319 | 94259465 |
| chrX | 94270263 | 94270387 |
| chrX | 94296571 | 94296697 |
| chrX | 94354727 | 94354861 |
| chrX | 94426020 | 94426144 |
| chrX | 94585665 | 94585951 |
| chrX | 94747559 | 94747748 |
| chrX | 94847236 | 94847430 |
| chrX | 95048010 | 95048150 |
| chrX | 95116265 | 95116397 |
| chrX | 95161490 | 95161616 |
| chrX | 95204539 | 95204663 |
| chrX | 95245868 | 95246008 |
| chrX | 95305065 | 95305197 |
| chrX | 95366884 | 95367010 |
| chrX | 95410521 | 95410665 |
| chrX | 95562260 | 95562410 |
| chrX | 95722242 | 95722368 |
| chrX | 95780186 | 95780348 |
| chrX | 95889538 | 95889662 |
| chrX | 95935787 | 95935919 |
| chrX | 95975885 | 95976009 |
| chrX | 95999221 | 95999345 |
| chrX | 96053704 | 96053852 |
| chrX | 96116055 | 96116181 |
| chrX | 96196800 | 96196934 |
| chrX | 96213171 | 96213297 |
| chrX | 96277250 | 96277396 |
| chrX | 96343620 | 96343744 |
| chrX | 96426349 | 96426489 |
| chrX | 96466095 | 96466223 |
| chrX | 96491120 | 96491250 |
| chrX | 96521782 | 96521960 |
| chrX | 96590868 | 96591156 |
| chrX | 96620314 | 96620573 |
| chrX | 96695036 | 96695170 |
| chrX | 96759511 | 96759649 |
| chrX | 96893406 | 96893682 |
| chrX | 96967957 | 96968089 |
| chrX | 97250381 | 97250602 |
| chrX | 97318852 | 97318972 |
| chrX | 97339116 | 97339237 |
| chrX | 97394795 | 97394933 |
| chrX | 97480850 | 97480992 |
| chrX | 97534762 | 97534886 |
| chrX | 97589371 | 97589499 |
| chrX | 97810558 | 97810690 |
| chrX | 97998400 | 97998544 |
| chrX | 98041067 | 98041189 |
| chrX | 98070935 | 98071075 |
| chrX | 98132882 | 98133016 |
| chrX | 98190180 | 98190304 |
| chrX | 98232711 | 98232851 |
| chrX | 98262248 | 98262374 |
| chrX | 98351816 | 98351942 |
| chrX | 98497407 | 98497528 |
| chrX | 98731935 | 98732077 |
| chrX | 98750383 | 98750573 |
| chrX | 98820988 | 98821244 |
| chrX | 98898311 | 98898474 |
| chrX | 98923054 | 98923182 |
| chrX | 98955966 | 98956215 |
| chrX | 99038221 | 99038343 |
| chrX | 99067121 | 99067247 |
| chrX | 99116235 | 99116401 |
| chrX | 99175307 | 99175449 |
| chrX | 99456155 | 99456279 |
| chrX | 99571878 | 99572008 |
| chrX | 99700718 | 99700864 |
| chrX | 99936936 | 99937060 |
| chrX | 100048786 | 100048920 |
| chrX | 100237773 | 100238049 |
| chrX | 100307321 | 100307457 |
| chrX | 100458607 | 100458731 |
| chrX | 100639725 | 100639875 |
| chrX | 100736725 | 100736849 |
| chrX | 100813160 | 100813284 |
| chrX | 100861593 | 100861723 |
| chrX | 100950521 | 100950645 |
| chrX | 101018125 | 101018265 |
| chrX | 101045433 | 101045561 |
| chrX | 101102385 | 101102523 |
| chrX | 101257637 | 101257758 |
| chrX | 101436652 | 101436786 |
| chrX | 101549164 | 101549286 |
| chrX | 101647710 | 101647832 |
| chrX | 101809426 | 101809713 |
| chrX | 101884435 | 101884561 |
| chrX | 101956580 | 101956701 |
| chrX | 102002189 | 102002460 |
| chrX | 102055318 | 102055497 |
| chrX | 102094454 | 102094588 |
| chrX | 102146188 | 102146316 |
| chrX | 102332885 | 102333006 |
| chrX | 102381205 | 102381333 |
| chrX | 102554133 | 102554257 |
| chrX | 102790345 | 102790487 |
| chrX | 102914300 | 102914426 |
| chrX | 102990952 | 102991102 |
| chrX | 103162899 | 103163025 |
| chrX | 103279802 | 103280025 |
| chrX | 103526786 | 103527048 |
| chrX | 103585439 | 103585565 |
| chrX | 103667611 | 103667767 |
| chrX | 103696783 | 103696905 |
| chrX | 103734285 | 103734421 |
| chrX | 103789174 | 103789298 |
| chrX | 103838439 | 103838567 |
| chrX | 103886670 | 103886791 |
| chrX | 103965174 | 103965314 |
| chrX | 104064451 | 104064593 |
| chrX | 104106642 | 104106796 |
| chrX | 104142842 | 104142968 |
| chrX | 104175322 | 104175452 |
| chrX | 104208300 | 104208422 |
| chrX | 104251856 | 104251980 |
| chrX | 104293698 | 104293818 |
| chrX | 104332150 | 104332272 |
| chrX | 104404580 | 104404838 |
| chrX | 104489935 | 104490178 |
| chrX | 104544396 | 104544678 |
| chrX | 104569010 | 104569134 |
| chrX | 104624181 | 104624378 |
| chrX | 104659210 | 104659332 |
| chrX | 104709772 | 104710050 |
| chrX | 104735521 | 104735778 |
| chrX | 104763276 | 104763522 |
| chrX | 104852875 | 104852996 |
| chrX | 104982563 | 104982834 |
| chrX | 105188402 | 105188536 |
| chrX | 105423870 | 105424114 |
| chrX | 105561073 | 105561291 |
| chrX | 105642888 | 105643174 |
| chrX | 105701648 | 105701910 |
| chrX | 105752814 | 105753062 |
| chrX | 105807655 | 105807940 |
| chrX | 105835738 | 105835859 |
| chrX | 105844950 | 105845070 |
| chrX | 105942538 | 105942800 |
| chrX | 106047061 | 106047222 |
| chrX | 106168011 | 106168153 |
| chrX | 106362621 | 106362761 |
| chrX | 106505368 | 106505498 |
| chrX | 106581121 | 106581257 |
| chrX | 106873806 | 106873930 |
| chrX | 107103093 | 107103219 |
| chrX | 107194307 | 107194429 |
| chrX | 107314082 | 107314220 |
| chrX | 107506181 | 107506305 |
| chrX | 107743296 | 107743436 |
| chrX | 107760057 | 107760199 |
| chrX | 107909390 | 107909512 |
| chrX | 108160558 | 108160688 |
| chrX | 108201174 | 108201304 |
| chrX | 108444791 | 108445085 |
| chrX | 108594007 | 108594129 |
| chrX | 108736283 | 108736425 |
| chrX | 108908270 | 108908399 |
| chrX | 108965568 | 108965702 |
| chrX | 109045650 | 109045778 |
| chrX | 109066072 | 109066333 |
| chrX | 109101284 | 109101524 |
| chrX | 109159637 | 109159923 |
| chrX | 109174563 | 109174839 |
| chrX | 109244821 | 109244961 |
| chrX | 109292112 | 109292236 |
| chrX | 109362891 | 109363017 |
| chrX | 109480838 | 109480958 |
| chrX | 109566040 | 109566160 |
| chrX | 109819535 | 109819798 |
| chrX | 109987010 | 109987140 |
| chrX | 110054029 | 110054303 |
| chrX | 110228698 | 110228946 |
| chrX | 110399083 | 110399419 |
| chrX | 110590595 | 110590841 |
| chrX | 110611200 | 110611768 |
| chrX | 110884971 | 110885151 |
| chrX | 111107493 | 111107778 |
| chrX | 111293434 | 111293621 |
| chrX | 111469839 | 111469965 |
| chrX | 111741419 | 111741561 |
| chrX | 111913674 | 111913798 |
| chrX | 112076861 | 112077013 |
| chrX | 112346749 | 112346887 |
| chrX | 112503910 | 112504036 |
| chrX | 112618680 | 112618806 |
| chrX | 112822144 | 112822298 |
| chrX | 112859336 | 112859476 |
| chrX | 113135700 | 113135822 |
| chrX | 113218257 | 113218391 |
| chrX | 113309871 | 113310005 |
| chrX | 113507242 | 113507377 |
| chrX | 113752741 | 113752895 |
| chrX | 114021493 | 114021748 |
| chrX | 114047870 | 114048139 |
| chrX | 114264862 | 114265133 |
| chrX | 114564073 | 114564205 |
| chrX | 114788487 | 114788641 |
| chrX | 114822647 | 114822773 |
| chrX | 114959253 | 114959373 |
| chrX | 115228144 | 115228284 |
| chrX | 115327040 | 115327178 |
| chrX | 115496468 | 115496612 |
| chrX | 115678701 | 115678822 |
| chrX | 115926042 | 115926210 |
| chrX | 116118780 | 116118914 |
| chrX | 116360961 | 116361117 |
| chrX | 116499937 | 116500063 |
| chrX | 116702273 | 116702450 |
| chrX | 116885698 | 116885822 |
| chrX | 117163126 | 117163273 |
| chrX | 117287118 | 117287246 |
| chrX | 117455384 | 117455532 |
| chrX | 117656803 | 117656957 |
| chrX | 117832183 | 117832313 |
| chrX | 118011402 | 118011524 |
| chrX | 118229043 | 118229173 |
| chrX | 118345485 | 118345756 |
| chrX | 118417410 | 118417650 |
| chrX | 118587285 | 118587409 |
| chrX | 118865042 | 118865170 |
| chrX | 119094450 | 119094612 |
| chrX | 119269738 | 119270004 |
| chrX | 119376601 | 119376873 |
| chrX | 119453959 | 119454101 |
| chrX | 119598195 | 119598587 |
| chrX | 119766412 | 119766560 |
| chrX | 119837989 | 119838113 |
| chrX | 119904578 | 119904700 |
| chrX | 119919088 | 119919214 |
| chrX | 119970343 | 119970536 |
| chrX | 120003853 | 120003974 |
| chrX | 120123902 | 120124060 |
| chrX | 120415453 | 120415589 |
| chrX | 120580388 | 120580528 |
| chrX | 120867914 | 120868040 |
| chrX | 120999751 | 120999895 |
| chrX | 121098982 | 121099278 |
| chrX | 121343969 | 121344125 |
| chrX | 121401707 | 121401847 |
| chrX | 121578076 | 121578200 |
| chrX | 121801083 | 121801209 |
| chrX | 121896647 | 121896771 |
| chrX | 122000615 | 122000741 |
| chrX | 122229503 | 122229631 |
| chrX | 122360282 | 122360542 |
| chrX | 122576297 | 122576445 |
| chrX | 122606558 | 122606735 |
| chrX | 122825400 | 122825658 |
| chrX | 122988157 | 122988424 |
| chrX | 123061975 | 123062115 |
| chrX | 123233345 | 123233485 |
| chrX | 123383031 | 123383181 |
| chrX | 123496372 | 123496496 |
| chrX | 123737427 | 123737551 |
| chrX | 123807055 | 123807181 |
| chrX | 124092262 | 124092549 |
| chrX | 124244642 | 124244766 |
| chrX | 124340563 | 124340809 |
| chrX | 124471986 | 124472128 |
| chrX | 124533020 | 124533176 |
| chrX | 124611828 | 124611968 |
| chrX | 124688607 | 124688747 |
| chrX | 124757355 | 124757489 |
| chrX | 124808199 | 124808349 |
| chrX | 124934441 | 124934567 |
| chrX | 125112138 | 125112395 |
| chrX | 125249450 | 125249598 |
| chrX | 125325200 | 125325344 |
| chrX | 125477225 | 125477357 |
| chrX | 125602960 | 125603086 |
| chrX | 125700282 | 125700408 |
| chrX | 125712816 | 125712972 |
| chrX | 125751662 | 125751796 |
| chrX | 125829717 | 125829889 |
| chrX | 125897172 | 125897301 |
| chrX | 125929800 | 125929921 |
| chrX | 125980590 | 125980728 |
| chrX | 126041426 | 126041550 |
| chrX | 126192786 | 126192908 |
| chrX | 126331131 | 126331255 |
| chrX | 126519784 | 126519916 |
| chrX | 126678563 | 126678684 |
| chrX | 126866824 | 126866946 |
| chrX | 127064080 | 127064320 |
| chrX | 127201668 | 127201790 |
| chrX | 127256530 | 127256704 |
| chrX | 127412112 | 127412236 |
| chrX | 127651073 | 127651211 |
| chrX | 127715134 | 127715274 |
| chrX | 127899769 | 127899899 |
| chrX | 128085513 | 128085647 |
| chrX | 128259401 | 128259543 |
| chrX | 128381331 | 128381469 |
| chrX | 128495643 | 128495767 |
| chrX | 128705787 | 128705907 |
| chrX | 128853487 | 128853625 |
| chrX | 129022727 | 129022848 |
| chrX | 129152534 | 129152777 |
| chrX | 129398475 | 129398605 |
| chrX | 129598847 | 129599015 |
| chrX | 129649697 | 129649847 |
| chrX | 129922892 | 129923145 |
| chrX | 129975239 | 129975520 |
| chrX | 130173584 | 130173852 |
| chrX | 130300573 | 130300705 |
| chrX | 130407234 | 130407388 |
| chrX | 130453856 | 130453996 |
| chrX | 130491346 | 130491468 |
| chrX | 130532049 | 130532183 |
| chrX | 130581208 | 130581332 |
| chrX | 130640392 | 130640534 |
| chrX | 130678764 | 130678892 |
| chrX | 130727908 | 130728052 |
| chrX | 130740663 | 130740793 |
| chrX | 130749736 | 130749912 |
| chrX | 130825940 | 130826076 |
| chrX | 130952951 | 130953077 |
| chrX | 131140819 | 131140943 |
| chrX | 131247921 | 131248149 |
| chrX | 131376496 | 131376636 |
| chrX | 131545311 | 131545554 |
| chrX | 131739744 | 131739866 |
| chrX | 131817843 | 131817965 |
| chrX | 131885843 | 131885977 |
| chrX | 131931399 | 131931543 |
| chrX | 131977864 | 131978002 |
| chrX | 131990899 | 131991023 |
| chrX | 132018696 | 132018818 |
| chrX | 132049061 | 132049195 |
| chrX | 132081792 | 132081914 |
| chrX | 132131006 | 132131138 |
| chrX | 132163613 | 132163737 |
| chrX | 132177114 | 132177238 |
| chrX | 132212239 | 132212369 |
| chrX | 132231156 | 132231425 |
| chrX | 132315102 | 132315376 |
| chrX | 132399277 | 132399399 |
| chrX | 132462767 | 132462905 |
| chrX | 132534699 | 132534831 |
| chrX | 132595279 | 132595417 |
| chrX | 132646058 | 132646184 |
| chrX | 132671541 | 132671709 |
| chrX | 132677023 | 132677145 |
| chrX | 132683288 | 132683636 |
| chrX | 132725459 | 132725589 |
| chrX | 132848738 | 132848935 |
| chrX | 133017781 | 133017949 |
| chrX | 133134351 | 133134509 |
| chrX | 133293999 | 133294131 |
| chrX | 133476612 | 133476735 |
| chrX | 133609627 | 133609970 |
| chrX | 133808830 | 133808964 |
| chrX | 133951733 | 133951935 |
| chrX | 134088129 | 134088261 |
| chrX | 134248790 | 134248965 |
| chrX | 134458649 | 134458803 |
| chrX | 134686288 | 134686457 |
| chrX | 134773894 | 134774146 |
| chrX | 134831625 | 134831753 |
| chrX | 134988221 | 134988342 |
| chrX | 135016579 | 135016826 |
| chrX | 135092984 | 135093312 |
| chrX | 135273305 | 135273477 |
| chrX | 135470433 | 135470571 |
| chrX | 135534884 | 135535024 |
| chrX | 135677656 | 135677780 |
| chrX | 135848695 | 135849026 |
| chrX | 135954793 | 135955041 |
| chrX | 136110795 | 136110935 |
| chrX | 136356675 | 136356797 |
| chrX | 136400543 | 136400667 |
| chrX | 136654448 | 136654715 |
| chrX | 136727436 | 136727574 |
| chrX | 136980234 | 136980360 |
| chrX | 137001020 | 137001174 |
| chrX | 137228032 | 137228158 |
| chrX | 137406512 | 137406634 |
| chrX | 137571732 | 137571856 |
| chrX | 137652215 | 137652339 |
| chrX | 137928126 | 137928250 |
| chrX | 138177629 | 138177753 |
| chrX | 138236608 | 138236748 |
| chrX | 138474537 | 138474661 |
| chrX | 138638885 | 138639053 |
| chrX | 138663328 | 138663620 |
| chrX | 138940285 | 138940406 |
| chrX | 139191048 | 139191188 |
| chrX | 139343892 | 139344057 |
| chrX | 139399559 | 139399682 |
| chrX | 139634852 | 139634986 |
| chrX | 139879736 | 139879870 |
| chrX | 139939774 | 139939896 |
| chrX | 139985216 | 139985368 |
| chrX | 140023070 | 140023192 |
| chrX | 140062186 | 140062307 |
| chrX | 140076871 | 140077101 |
| chrX | 140087328 | 140087458 |
| chrX | 140099333 | 140099463 |
| chrX | 140209388 | 140209508 |
| chrX | 140334003 | 140334133 |
| chrX | 140388010 | 140388164 |
| chrX | 140559210 | 140559330 |
| chrX | 140670217 | 140670347 |
| chrX | 140680347 | 140680477 |
| chrX | 140783981 | 140784111 |
| chrX | 140802883 | 140803150 |
| chrX | 140964803 | 140964953 |
| chrX | 141044877 | 141045003 |
| chrX | 141086572 | 141086696 |
| chrX | 141126566 | 141126724 |
| chrX | 141204489 | 141204615 |
| chrX | 141271721 | 141271850 |
| chrX | 141399081 | 141399203 |
| chrX | 141441798 | 141441922 |
| chrX | 141506087 | 141506211 |
| chrX | 141765392 | 141765562 |
| chrX | 141802595 | 141802745 |
| chrX | 142052308 | 142052460 |
| chrX | 142128370 | 142128492 |
| chrX | 142245439 | 142245595 |
| chrX | 142506358 | 142506486 |
| chrX | 142682546 | 142682672 |
| chrX | 142776613 | 142776735 |
| chrX | 143002106 | 143002238 |
| chrX | 143210414 | 143210540 |
| chrX | 143259813 | 143259937 |
| chrX | 143441178 | 143441300 |
| chrX | 143499445 | 143499583 |
| chrX | 143569581 | 143569709 |
| chrX | 143582515 | 143582758 |
| chrX | 143625458 | 143625580 |
| chrX | 143697896 | 143698022 |
| chrX | 143735844 | 143735968 |
| chrX | 143771445 | 143771567 |
| chrX | 143782812 | 143782934 |
| chrX | 143862188 | 143862322 |
| chrX | 143942225 | 143942369 |
| chrX | 144155493 | 144155623 |
| chrX | 144209429 | 144209555 |
| chrX | 144370005 | 144370132 |
| chrX | 144528713 | 144528855 |
| chrX | 144706634 | 144706758 |
| chrX | 144816681 | 144816805 |
| chrX | 145069448 | 145069572 |
| chrX | 145186004 | 145186154 |
| chrX | 145335834 | 145335970 |
| chrX | 145425475 | 145425615 |
| chrX | 145643788 | 145643914 |
| chrX | 145764920 | 145765068 |
| chrX | 145989688 | 145989818 |
| chrX | 146002713 | 146002873 |
| chrX | 146251345 | 146251618 |
| chrX | 146322571 | 146322693 |
| chrX | 146446565 | 146446703 |
| chrX | 146693171 | 146693301 |
| chrX | 146823440 | 146823584 |
| chrX | 146899753 | 146899897 |
| chrX | 147085094 | 147085236 |
| chrX | 147318920 | 147319044 |
| chrX | 147372926 | 147373078 |
| chrX | 147640346 | 147640705 |
| chrX | 147770027 | 147770151 |
| chrX | 147904458 | 147904945 |
| chrX | 148081653 | 148081819 |
| chrX | 148149449 | 148149579 |
| chrX | 148266662 | 148266924 |
| chrX | 148428078 | 148428319 |
| chrX | 148594987 | 148595256 |
| chrX | 148837765 | 148837887 |
| chrX | 148901208 | 148901329 |
| chrX | 148977214 | 148977335 |
| chrX | 149006458 | 149006715 |
| chrX | 149030925 | 149031055 |
| chrX | 149085129 | 149085346 |
| chrX | 149141442 | 149141568 |
| chrX | 149336541 | 149336710 |
| chrX | 149466940 | 149467074 |
| chrX | 149624724 | 149624864 |
| chrX | 149800414 | 149800758 |
| chrX | 150007704 | 150007825 |
| chrX | 150088712 | 150088850 |
| chrX | 150214722 | 150214846 |
| chrX | 150265044 | 150265176 |
| chrX | 150322460 | 150322584 |
| chrX | 150391483 | 150391613 |
| chrX | 150478761 | 150478897 |
| chrX | 150544239 | 150544363 |
| chrX | 150684860 | 150684984 |
| chrX | 150757665 | 150757931 |
| chrX | 150792644 | 150792897 |
| chrX | 150813336 | 150813606 |
| chrX | 150847821 | 150848068 |
| chrX | 150920423 | 150920547 |
| chrX | 150990322 | 150990470 |
| chrX | 151191434 | 151191580 |
| chrX | 151373796 | 151373920 |
| chrX | 151413879 | 151414021 |
| chrX | 151457544 | 151457668 |
| chrX | 151503645 | 151503775 |
| chrX | 151563866 | 151563992 |
| chrX | 151605080 | 151605218 |
| chrX | 151667577 | 151667727 |
| chrX | 151749017 | 151749297 |
| chrX | 151911207 | 151911379 |
| chrX | 152130370 | 152130512 |
| chrX | 152350467 | 152350613 |
| chrX | 152477929 | 152478069 |
| chrX | 152559904 | 152560124 |
| chrX | 152602659 | 152602787 |
| chrX | 152651695 | 152651827 |
| chrX | 152736628 | 152736756 |
| chrX | 152747584 | 152747738 |
| chrX | 152856131 | 152856255 |
| chrX | 152973151 | 152973271 |
| chrX | 153022238 | 153022366 |
| chrX | 153077170 | 153077292 |
| chrX | 153327588 | 153327929 |
| chrX | 153505426 | 153505593 |
| chrX | 153555330 | 153555472 |
| chrX | 153592163 | 153592287 |
| chrX | 153627456 | 153627578 |
| chrX | 153713741 | 153713871 |
| chrX | 153737350 | 153737543 |
| chrX | 153773171 | 153773292 |
| chrX | 153776178 | 153776425 |
| chrX | 153827652 | 153827831 |
| chrX | 153885781 | 153886006 |
| chrX | 154002334 | 154002505 |
| chrX | 154155638 | 154156005 |
| chrX | 154336620 | 154336748 |
| chrX | 154454035 | 154454175 |
| chrX | 154664177 | 154664299 |
| chrX | 154777476 | 154777630 |
| chrX | 155257996 | 155258121 |
| chrY | 2692665 | 2692785 |
| chrY | 2819274 | 2819395 |
| chrY | 3074606 | 3074734 |
| chrY | 3162349 | 3162469 |
| chrY | 3196339 | 3196475 |
| chrY | 3339187 | 3339325 |
| chrY | 3696256 | 3696382 |
| chrY | 3719879 | 3720001 |
| chrY | 3798834 | 3798984 |
| chrY | 3978337 | 3978465 |
| chrY | 4056546 | 4056690 |
| chrY | 4196075 | 4196211 |
| chrY | 4360019 | 4360177 |
| chrY | 4437146 | 4437270 |
| chrY | 4586405 | 4586526 |
| chrY | 4902439 | 4902561 |
| chrY | 5075970 | 5076110 |
| chrY | 5171704 | 5171826 |
| chrY | 5298853 | 5298975 |
| chrY | 5529192 | 5529342 |
| chrY | 5611518 | 5611640 |
| chrY | 5747157 | 5747297 |
| chrY | 5970582 | 5970706 |
| chrY | 6517111 | 6517235 |
| chrY | 6630949 | 6631070 |
| chrY | 6793046 | 6793208 |
| chrY | 6970809 | 6970958 |
| chrY | 7070574 | 7070694 |
| chrY | 7152066 | 7152189 |
| chrY | 7325184 | 7325347 |
| chrY | 7560166 | 7560289 |
| chrY | 7742165 | 7742320 |
| chrY | 7887631 | 7887752 |
| chrY | 7901532 | 7901654 |
| chrY | 8180136 | 8180256 |
| chrY | 8348119 | 8348240 |
| chrY | 8489265 | 8489587 |
| chrY | 8584697 | 8584820 |
| chrY | 8714552 | 8714674 |
| chrY | 8858599 | 8858719 |
| chrY | 9424906 | 9425029 |
| chrY | 9644794 | 9644914 |
| chrY | 9988878 | 9989001 |
| chrY | 13247930 | 13248062 |
| chrY | 13318311 | 13318449 |
| chrY | 13639786 | 13639934 |
| chrY | 14026357 | 14026524 |
| chrY | 14044532 | 14044658 |
| chrY | 14071903 | 14072028 |
| chrY | 14081438 | 14081559 |
| chrY | 14096264 | 14096384 |
| chrY | 14177413 | 14177534 |
| chrY | 14277544 | 14277701 |
| chrY | 14469471 | 14469593 |
| chrY | 14650154 | 14650275 |
| chrY | 14752266 | 14752464 |
| chrY | 14884036 | 14884199 |
| chrY | 15079019 | 15079139 |
| chrY | 15343175 | 15343299 |
| chrY | 15460700 | 15460830 |
| chrY | 15666638 | 15666761 |
| chrY | 15814194 | 15814314 |
| chrY | 15930364 | 15930558 |
| chrY | 16131550 | 16131671 |
| chrY | 16192441 | 16192592 |
| chrY | 16402022 | 16402143 |
| chrY | 16582473 | 16582594 |
| chrY | 16630321 | 16630444 |
| chrY | 16887545 | 16887668 |
| chrY | 16919789 | 16919911 |
| chrY | 17055101 | 17055222 |
| chrY | 17252062 | 17252265 |
| chrY | 17427731 | 17427856 |
| chrY | 17506978 | 17507100 |
| chrY | 17705136 | 17705347 |
| chrY | 17875186 | 17875320 |
| chrY | 18066076 | 18066204 |
| chrY | 18151331 | 18151460 |
| chrY | 18260215 | 18260335 |
| chrY | 18548074 | 18548200 |
| chrY | 18634399 | 18634521 |
| chrY | 18723469 | 18723590 |
| chrY | 18964173 | 18964311 |
| chrY | 19037493 | 19037614 |
| chrY | 19224195 | 19224433 |
| chrY | 19404889 | 19405009 |
| chrY | 19506262 | 19506398 |
| chrY | 19565853 | 19566048 |
| chrY | 20839387 | 20839541 |
| chrY | 21039998 | 21040119 |
| chrY | 21230586 | 21230708 |
| chrY | 21370616 | 21370751 |
| chrY | 21481113 | 21481248 |
| chrY | 21683036 | 21683156 |
| chrY | 21752319 | 21752451 |
| chrY | 21986061 | 21986184 |
| chrY | 22116478 | 22116632 |
| chrY | 22206699 | 22206820 |
| chrY | 22567907 | 22568031 |
| chrY | 22607462 | 22607582 |
| chrY | 22835982 | 22836121 |
| chrY | 22913465 | 22913587 |
| chrY | 23140262 | 23140382 |
| chrY | 23313437 | 23313559 |
| chrY | 23379718 | 23379839 |
| chrY | 23455385 | 23455514 |
| chrY | 24874078 | 24874199 |
| chrY | 25534198 | 25534324 |
| chrY | 26525571 | 26525717 |
| chrY | 27436715 | 27436861 |
| chrY | 28428589 | 28428715 |
| chrY | 58986552 | 58986673 |
| chrY | 59361002 | 59361127 |

Column 1 = Chromosome No.;
Column 2 = Start Position;
Column 3 = Stop Position

RRPH provides for analysis of all of the common genes used for risk stratification of AML including ASXL1, CEBPA, DNMT3A, FLT3, IDH1, IDH2, KIT, KRAS, NPM1, NRAS, RUNX1, and TP53.

For myeloproliferative neoplasms mutations in JAK2, CALR, and SH2B3 are provided. Like FLT3 with the hybridization capture approach the classical 52 base pair exon 9 deletion (Type 1) in CALR as well as the 5 base pair insertion (Type 2) mutations are readily identified.

RPPH like no other advanced diagnostic assay can simultaneously identify gene specific mutations, amplification, or deletion of specific genes, and associated cytogenetic alterations simultaneously. Examples of such alterations in myeloid neoplasms include mutations and deletions in the following genes with the associated cytogenetic alteration, including EGR1 and loss of 5q31 or −5 or del(5q), CSF1R and loss of 5q33-q34, ATM and loss of 11q23, TP53 and loss of 17p13 or −17, CDKN2A and loss of 9p21 or −9, and PTEN and loss of 10q23. Similar associations for chronic lymphocytic leukemia (CLL) applicable to RPPH and not previously mentioned include RB1 and loss of 13q14, and POT1 and loss of 7q31. Applicable to the latter tumor type as a single cytogenetic change at the chromosomal level is the ability or RPPH to identify trisomy 12. In a similar model of trisomy 12, RPPH can identify gain of odd numbered chromosomes, so-called hyper diploid phenotype, in multiple myeloma.

The RNA component of the assay was designed and developed using specific databases to identify key fusion genes important to hematological malignancies. This custom RNA hybridization capture assay allows for the fusion analysis of 125 genes associated with hematological malignancies without the need for prior knowledge of the fusion partners or the breakpoints of the translocations when a chimeric transcript is formed, including ABL1, ABL2, ACTB, AFF1, AHI1, ALK, ASXL1, ASXL2, ATF7IP, ATM, BCL11B, BCL2, BCL6, BCOR, BCR, BIRC3, BMI1, BTK, CALR, CBFB, CBFA2T3, CBL, CCND1, CCND3, CD28, CDK6, CDKN2C, CEP43, CEBPA, CHD1, CHEK2, CHIC2, CIITA, CREBBP, CRLF2, CSF1R, CUX1, CXCR4, DAZAP1, DDX6, DDX10, DDX3X, DDX41, DEK, DIS3, DNMT3A, DSCAM, DUSP22, EBF1, EGR1, EIF4A1, ELL, EP300, EPOR, ERG, ETS2, ETV6, FGFR1, FGFR3, FLT3, FOXP1, FOXR1, FUS, GATA2, GLIS2, H2AX, HNRNPC, HOXA9, HOXA11, IDH1, IDH2, IGH, IGK, IGL, IKZF1, IKZF2, IKZF3, IRF4, JAK2, KANSL1, KAT6A, KDM5A, KLF2, KMT2A, KMT2C, KRAS, LMO1, LMO2, LYN, MALT1, MAP2K1, MECOM, MEK, MLF1, MLLT4, MLLT10, MKL1, MPL, MN1, MPO, MRTFA, MSI2, MTCP1, MYB, MYC, MYH9, MYH111, NF1, NFIA, NFE2, NFKB2, NIPBL, NOTCH1, NPM1, NRAS, NSD1, NTRK3, NUP214, NUP98, NUTM1, P2RY8, PAG1, PAX5, PBX1, PDCD1LG2, PDGFRA, PDGFRB, PHF6, PICALM, PLCG2, PML, PPM1D, PRDM16, PRRC2B, PTK2B, RARA, RARG, RB1, RBM15, RET, ROS1, RNF213, RUNX1, RUNX1T1, SEC31A, SETBP1, SEMA6A, SEPTIN9, SETD2, SF3B1, SH2B3, SPI1, SRSF2, SSBP2, STAG2, STAT3, STAT6, STIL, SYK, TAL1, TBL1XR1, TCF3, TCL1A, TFG, TP63, TP73, TRAF2, TPM3, TPM4, TRA, TRB, TRD, TYK2, U2AF1, UBA2, VAV1, WT1, XPO1, YPEL5, ZCCHC7, ZFP36L2, ZNF362, ZNF384, and ZRSR2. The ability to detect all fusion partners for this list of driver genes expands the list of possible genes interrogated as some genes like KMT2A (MLL) can have up to 60 different fusion partner genes. For NHL that are characterized by distinctive fusions that do not produce a chimeric transcript, such as CCND1-IGH t(11;14)(q13;q32) frequently identified in mantle cell lymphoma (MCL) and MM, FGFR3-IGH t(4;14)(p16;q32) in MM, BCL6-IGH t(3;14)(q27;q32) and BCL2-IGH t(14;18)(q32;q21) in diffuse large B-cell lymphoma (DLBCL) and follicular lymphoma (FL), MYC-IGH t(8;14)(q24;q32) in DLBCL, Burkitt lymphoma (BL), and FL, and MALT1-IGH t(14;18)(q32;q21) and API2-MALT1 t(11;18)(q21;q21) in mucosal-associated lymphoma (MALT), the only mode of detection is over expression of the driver gene (non-IGH component). For leukemias that are characterized by distinctive fusions that produce a chimeric transcript, RPPH provides analysis of the t(12;21)(p12;q22) ETV6-RUNX1(TEL-AML1), t(1;19)(q23;p13) TCF3-PBX1 (E2A-PBX) and t(4;11)(q21;q23) AF1-MLL frequently identified in B-acute lymphocytic leukemia (B-ALL), t(9;22)(q34.1;q11) ABL1-BCR in chronic myelogenous leukemia (CML) and B-ALL, t(8;21)(q22;q22) RUNX1-RUNX1T1 (AML1-ETO) in acute myelogenous leukemia (AML) M2, t(15;17)(q22;q12) PML-RARA in AML-M3, and t(16;16)(p13;q22)/inv(16)(p13q22) MYH11-CBFB in AML-M4EO. RPPH also provides analysis of chimeric fusions commonly identified in therapy related AML such as t(5;12)(q31;p13) ETV6-ACSL6, t(9;12)(q22;p13) ETV6-SYK, t(10;16)(q22;p13) KAT6B-CREBBP, t(11;19)(q23;q13) KMT2A-ACTN4, t(11;16)(q23;p13.3) KMT2A-CREBBP, t(11;19)(q23;p13.1) KMT2A-ELL, t(9;11)(p21;q23) KMT2A-MLLT3, t(11;16)(q23;p13) KMT2A-MYH11, t(16;21)(q24;q22) RUNX1-CBFA2T3, t(3;21)(q26;q22) RUNX1-MECOM, t(7;21)(p22;q22) RUNX1-USP42, and t(8;21)(q23;q22) RUNX1-ZFPM2.

While somewhat more uncommon chimeric fusions previously associated with myeloproliferative neoplasms such as t(5;9)(q32;p24) KANK1-PDGFRB, ins(12;8)(p13;q11q21) ETV6-LYN, t(8;9)(p22;p24) PCM1-JAK2, t(1;5)(q21;q32) PDE4DIP-PDGFRB, and t(3;5)(q13;q32) GOLGB1-PDGFRB are also readily identified. Many of the these previously listed fusions have been identified in myelodysplastic syndrome (MDS), but others such as t(5;12)(q31;p13) ETV6-ACSL6, t(9;12)(q22;p13) ETV6-SYK, and t(6;9)(p22;q34) DEK-NUP214 are also covered by RPPH. In summary, RPPH covers all the canonical chimeric fusions, but this represents only a small percentage of the breadth of fusions that can be potentially identified.

To summarize, RPPH is an advanced NGS-based diagnostic test that is applicable to all hematological neoplasms including acute and chronic leukemia, MDS, MPN, NHL, and MM. RPPH combines multiple cutting-edge technologies that have never been combined before in a single assay that will allow for a deeper and more thorough understanding of the genomic profile of all hematological neoplasms. This process includes custom analysis pipelines developed by Roswell Park Department of Pathology for variant detection and integration into a comprehensive report.

### Example 2: Acute Myeloid Leukemia Genetics Panel

Acute myeloid leukemia (AML) is a heterogeneous group of diseases that represent clonal proliferations of immature, nonlymphoid, bone marrow-derived cells that typically involve both the bone marrow and peripheral blood. AML classification by World Health Organization (WHO) schema incorporates recurrent genetic changes as an important variable in addition to morphology and immunophenotype, with “AML with recurrent genetic abnormalities” and “AML with myelodysplasia related changes” being two of five major classifications.

Within the category of AML with recurrent genetic abnormalities there are seven classes based upon fusions and three for mutations.
AML with t(8;21)(q22q22.1); RUNX1-RUNX1T1
AML with inv(16)(p13.1q22) or t(16;16)(p13.1;q22);CBFB-MYH11
APL with PML-RARA
AML with t(9;11)(p21.3;q23.3); KMT2A-MLLT3
AML with t(6;9)(p23;q34.1); DEK-NUP214
AML with inv(3)(q21.3q26.2) or t(3;3)(q21.3;q26.2); GATA2, MECOM
AML (megakaryoblastic) with t(1;22)(p13.3;q13.1); RBM15-MKL1
Provisional entity: AML with BCR-ABL1
AML with mutated NPM1
AML with biallelic mutation of CEBPA
Provisional entity: AML with mutated RUNX1

In addition to the classification of AML with recurrent genetic abnormalities by specific fusion or mutation events other associated genetic variants can influence prognosis and treatment.

RUNX1-RUNX1T7 AML Genetic Associations: Mutations in the gene KIT in exons 8 and 17 portend a worse prognosis. Additional structural variants, including a loss or X or Y or 9q-, are also associated with a worse prognosis.

CBFB-MYH11 AML Genetic Associations: Mutations in the gene KIT present in approximately 30% of this group portend a worse prognosis.

PML-RARA AML Genetic Associations: FLT3 internal tandem duplications (ITD) occur in up to 40% of this group and are associated with a higher death rate during induction chemotherapy. Patients with RARA fusion partners other than PML have a high risk of disseminated intravascular coagulation.

KMT2A-MLLT3 AML Genetic Associations: KMT2A, better known as MLL, has multiple translocation partners other than MLLT3 (AF9), including MLLT10 (AF10), ELL, MLLT4 (AF6), and MLLT1 (ENL) and classified as AML with balanced translocation 11q23 MLL, NOS, KIT mutations and FLT3 ITD are uncommon and of uncertain significance.

DEK-NUP2/4 AML Genetic Associations: An uncommon variant of AML with a poor prognosis DEK NUP214 frequently has FLT3 ITD, although the prognostic impact of this mutational change is not clearly defined.

GATA2 (RPN1), MECOM (EVI1) AML (Genetic Associations: Complex karyotypes with −7 and −5q are associated with a poor prognosis.

RBM15-MKL1 AML Genetic Associations: Associated genetic changes are not well known in this rare subtype of AML typically seen in infants.

NPM1 Mutated AML Genetic Associations: Found in 50% of adult and 20% pediatric normal karyotype AML and in the absence of FLT3 ITD, which constitutes 60% of cases, has a favorable prognosis. Structural variants are uncommon in NPM1 mutated AML.

CEBPA Mutated AML Genetic Associations: Found in 15 to 200% of adult and pediatric normal karyotype and in the absence of FLT3 ITD, which are uncommon, has a favorable prognosis with CEBPA double mutations. CEBPA mutated AML is unlikely to benefit from allogeneic stem cell transplantation.

In addition to recurrent genetic changes currently in the WHO classification there are other AML with similar changes including acute lymphoblastic leukemia (ALL) with BCR-ABL1 fusion, MLF1-NPM1 fusion positive AML, and MYST3-CREBB fusion positive AML.

AML with myelodysplasia related changes is typically associated with a complex karyotype and poor risk structural variants that include −5/5q-, −7/7q-, 9q-, 11q-, 12p-, and −13/13q-. In addition to structural variants specific fusions including, KMT2A/CREBBP, RUNX1/MECOM, HIP1/PDGFRB, RABEP1/PDGFRB, CCDC6/PDGFRB, and NPM1/MLF1 are other criteria for AML with myelodysplasia related changes.

### CBF AML

Beyond WHO classification there are other genetic changes that impact treatment and or prognosis. Most common is a “complex karyotype”, defined as 3 or more numerical or chromosomal structural variants. Most commonly and associated with a poor prognosis are losses or structural variants of chromosomes 5, 7, and 17. Patients with these structural variants combined with a TP53 mutation have particularly lower remission rates and poorer survival.

Beyond recurrently mutated FLT3, CEBPA, or NPM1 AML mutations in other genes including WT1, ASXL1, ASXL2, BCOR, DNMT3A, EZH2, KIT, MLL, PHF6, SRSF2, SF3B1, STAG2, TP53, U2AF1, and ZRSR2 have an unfavorable prognosis.

US Food and Drug Administration targeted therapies are approved for FLT3 and IDH2.

### Testing

Identification of one or more recurrent genetic abnormalities results in establishing the diagnosis and subclassification of AML
Identification of one or more recurrent genetic abnormalities aids in prognostication and clinical management of patients with AML
Specific mutations, cytogenetic abnormalities, or fusions has diagnostic or prognostic significance and informs clinical management
Specific mutations are associated with targeted therapies.

### Genetics

TABLE 1
Genes Tested for Mutations (single nucleotide
variants, insertions, deletions)

| | | | | |
|---|---|---|---|---|
| ABL1 | CREBBP | IDH2 | NPM1 | STAG2 |
| ASXL1 | DNMT3A | JAK2 | PHF6 | TP53 |
| ASXL2 | EZH2 | KIT | RUNX1 | U2AF1 |
| BCOR | FLT3 | KMT2A | SF3B1 | WT1 |
| CEBPA | IDH1 | MECOM | SRSF2 | ZRSR2 |

TABLE 2
Genes Tested for Copy Number Variants

| | | |
|---|---|---|
| ATM | RB1 | TP53 |

TABLE 3
Fusion Genes Tested

| | | | |
|---|---|---|---|
| ABL1 | FGFR1 | MYH11 | RARA |
| BCR | KMT2A | NUP214 | RBM15 |
| CBFB | MECOM | PDGFRA | RUNX1 |
| CREBBP | MKL1 | PDGFRB | RUNX1T1 |
| DEK | MLF1 | PML | |

### Results

### Genes Tested for Mutations

Positive: a somatic mutation in one of the tested genes was detected
Negative: no mutations were detected in the sequenced genes

### Genes Tested for Copy Number Variants

Positive: a copy number variant in one of the tested genes was detected
Negative: no copy number variants were detected in the sequenced genes

### Fusions Tested

Positive: a fusion involving one of the tested genes was detected
Negative: no fusions were detected in the sequenced genes

TABLE 4
Analytical Sensitivity

| | Variant Allelic | Analytical |
|---|---|---|
| Variant Class | Frequency | Sensitivity |
| Single nucleotide | 5% | 95% |
| variants | | |
| Insertions | 5% | 95% |
| deletions < 20 bp | | |
| Insertions | 5% | 95% |
| deletions => 20 bp | | |
| Fusions | 5% | 95% |

### Specimen Requirements

Peripheral blood: 5 mL in EDTA tube.
Bone marrow: 2 mL in EDTA tube.

TABLE 5
Applicable AMLs

| | |
|---|---|
| C92.00 -hyphen C92.02 | Acute myeloid leukemia (AML) |
| C92.40 -hyphen C92.A2 | |
| C91.00 -hyphen C91.02 | Acute lymphoblastic leukemia [ALL] |
| C83.50 -hyphen C83.59 | Lymphoblastic (diffuse) lymphoma |
| C91.00 -hyphen C91.02 | Acute myeloblastic leukemia |
| C91.10 -hyphen C91.12 | Chronic lymphocytic leukemia of B-hyphencell type |
| C92.00 -hyphen C92.12 | Myeloid leukemia |
| C92.20 -hyphen C92.62 | Atypical chronic myeloid leukemia BCR/ABL - negative, |
| | myeloid sarcoma, acute promyelocytic leukemia, acute |
| | myelomonocytic leukemia, and acute myeloid leukemia |
| | with 11q23-hyphenabnormality |
| C92.A0 -hyphen C92.A2 | Acute myeloid leukemia with multilineage dysplasia |
| C92.Z0 -hyphen C92.Z2 | Other myeloid leukemia |

### Example 3: Myelodysplasia Genetics Panel

The myelodysplastic syndromes (MDS) comprise a group of hematologic malignancies characterized by clonal hematopoiesis, one or more cytopenias (ie, anemia, neutropenia, and/or thrombocytopenia), and abnormal cellular maturation. MDS shares clinical and pathologic features with acute myeloid leukemia (AML), but MDS has a lower percentage of blasts in peripheral blood and bone marrow (by definition, <20 percent). Patients with MDS are at risk for symptomatic anemia, infection, bleeding, and transformation to AML.

In addition to cytopenias the diagnosis of MDS and prognosis based upon the Revised International Prognostic Scoring System (IPSS-R) has historically been based upon chromosomal abnormalities including, but not limited to loss of chromosome 7 or del(7q), del(5q), t(5q), isochromosome 17q or t(l 7p), loss of chromosome 13 or del(13q), del(11q), del(12p) or t(l 2p), del(9q), idic(X)(q13), t(I 116)(23.3;p13.3); KMT2A-CREBBP, t(3;21)(q26.2;q22.1); RUNX11-EVI1, t(1;3)(p36.3;q21.2); PSMD2-PRDM16, t(2;11)(p21;q23.3); KMT2A-?, inv(3)(q211q26.2) or t(3;3)(q21.2;q26.2); RPN1-EVI1, and t(6;9)(p23;q34.1); DEK-NUP214.

Additionally, specific chromosomal abnormalities exclude MDS, including t(8;21)(q22;q22); RUNX1-RUNX1T1 (previously AML1-ETO), inv(16)(p13.1q22) or t(6;16)(p13.1;q22); CBFB-MYH111, and t(15,17)(q22;q21.1), PML-RARA.

Among this group of chromosomal abnormalities deletion of the long arm of chromosome 5 (5q) is the most common chromosomal abnormality seen in MDS, occurring in approximately 15 percent of cases overall. The identification of an isolated del(5q), or del(5q) plus one other abnormality, with the exception of −7/del(7q), is used in the World Health Organization (WHO) classification system of MDS to define the 5q-syndrome. Deletions of 5q are further defined by deletion of 5q32-33.1 that are associated with a good prognosis, and 5q31.2 deletion that is more commonly seen with high risk MDS or therapy-related MDS and is associated with a complex karyotype, TP53 loss or mutation, and more aggressive disease.

In addition to the diagnosis of MDS using specific recurrent genetic chromosomal abnormalities there are additional mutational events that influence prognosis and treatment, including:
Adverse—ASXL1, BCOR, CBL, DNMT3A, EZH2, FLT3, NRAS, RUNX1, SRSF2, STAG2, TP53, U2AF1
Favorable—SF3B1
Neutral or uncertain—IDH1, IDH2, TET2, ZRSR2

Among this group mutations in EZH2, SF3B1, and TP53 have been utilized to form a molecular IPSS-R. Mutations in other genes support specific subtypes of MDS or differential diagnoses such as CBL and NF1 in CMML or JMML, JAK2, CALR, MPL in MDS/MPN-RS&T

### Testing

Identification of one or more recurrent genetic abnormalities results in establishing the diagnosis of MDS
Identification of one or more recurrent genetic abnormalities aids in prognostication and clinical management of patients with MDS
Specific mutations, cytogenetic abnormalities, or fusions has diagnostic or prognostic significance and inform clinical management

### Genetics

TABLE 6
Genes Tested for Mutations (single nucleotide
variants, insertions, deletions)

| | | | | | |
|---|---|---|---|---|---|
| ASXL1 | DNMT3A | IDH1 | NRAS | SF3B1 | TP53 |
| BCOR | ETV6 | IDH2 | PHF6 | SRSF2 | U2AF1 |
| CALR | EZH2 | JAK2 | PPM1D | STAG2 | WT1 |
| CBL | FLT3 | MPL | RUNX1 | STAT3 | ZRSR2 |
| DDX41 | GATA2 | NPM1 | SETBP1 | TET2 | |

TABLE 7
Genes Tested for Copy Number Variants

| | |
|---|---|
| ATM | RB1 |

TABLE 8
Fusion Genes Tested

| | | |
|---|---|---|
| CBFB | MECOM | PRDM16 |
| CREBBP | MYH11 | RARA |
| DEK | NUP214 | RUNX1 |
| KMT2A | PML | RUNX1T1 |

### Results

### Genes Tested for Mutations

Positive: a somatic mutation in one of the tested genes was detected
Negative: no mutations were detected in the sequenced genes

### Genes Tested for Copy Number Variants

Positive: a copy number variant in one of the tested genes was detected
Negative: no copy number variants were detected in the sequenced genes

### Fusions Tested

Positive: a fusion involving one of the tested genes was detected
Negative: no fusions were detected in the sequenced genes

TABLE 9
Analytical Sensitivity

| | Variant Allelic | Analytical |
|---|---|---|
| Variant Class | Frequency | Sensitivity |
| Single nucleotide | 5% | 95% |
| variants | | |
| Insertions | 5% | 95% |
| deletions < 20 bp | | |
| Insertions | 5% | 95% |
| deletions => 20 bp | | |
| Fusions | 5% | 95% |

### Specimen Requirements

Peripheral blood: 5 mL in EDTA tube.
Bone marrow: 2 mL in EDTA tube.

TABLE 10
Applicable MDSs

| | |
|---|---|
| D46.0 -hyphen D46.9 | Myelodysplastic syndromes |

### Example 4: Myeloproliferative Neoplasms Genetics Panel

BCR/ABL1-negative myeloproliferative neoplasms (MPNs) cause excess blood cell production in the bone marrow and are characterized by recurrent genetic changes. BCR/ABL1-negative MPNs include polycythemia vera (PV), essential thrombocythemia (ET), primary myelofibrosis (PMF), chronic neutrophilic leukemia (CNL), and chronic eosinophilic leukemia (CEL). The majority of patients with PV, ET, and PMF have a mutation in the JAK2, CALR, or MPL gene, which carries implications for classification, diagnosis, and prognosis, however when negative other recurrent genetic changes may also be diagnostic for these neoplasms.

PV is noted for JAK2 V716F mutation in more than 95% of cases, with other JAK2 exon 12 or 14 mutations in V617F negative cases. In addition to JAK2 mutations 10 to 20% of cases will have recurrent structural variants that include +8, +9, 20q-, 13q-, and 9p-. There is an increased incidence of structural variants associated with disease progression.

ET is noted for JAK2 V716F mutation in approximately 50% of cases, with 1 to 2% 5% having mutations in MPL with W515L/K being most common. JAK2 and MPL negative ET frequently have exon 9 insertions or deletions in the gene CALR, structural variants are uncommon, present in less than 10% of cases, and typically include +8 or del(20q).

PMF is noted for JAK2 V716F mutation in approximately 50% of cases, with a smaller number estimated at 5% having mutations in MPL with W515L/K being most common. JAK2 and MPL negative PMF frequently have exon 9 insertions or deletions in the gene CALR A diversity of nonrecurrent cytogenetic abnormalities have been reported in PMF including −5/5q-, −7/7q-, +8, +9, 12p-, 13q-, and 20q-. When present as a sole abnormality 13q- and 20q- are considered favorable, while +8 is unfavorable. Transformation to MDS or AML is typically associated with complex cytogenetic abnormalities that typically include −5/5q- and or −7/7q-.

CEL, and other more uncommon eosinophilic leukemic disorders, are noted for gene fusions rather than mutations or other cytogenetic abnormalities. Classic CEL is noted for a cryptic deletion of part of chromosome 4q12 resulting in a FIP1L1/PDGFRA fusion. A number of leukemic disorders with eosinophilic features are noted for various fusions associated with the gene PDGFRB at 12p12, with ETV6/PDGFRB being the most common. Other less common fusions in eosinophilic leukemic disorders other than CEL are associated with the gene FGFR1. Located at 8p11, FGFR1 has several partner genes, such as MYST3 and WHSC1L1, in so-called 8p11 myeloproliferative syndrome.

CNL is a rare MPN not noted for recurrent mutations or fusions, although JAK2 V617F has been previously identified. Reported recurrent cytogenetic abnormalities include +8, +9, 11q-, 20q-, and +21.

### Testing

Identification of one or more recurrent genetic abnormalities results in establishing the diagnosis and subclassification of a myeloproliferative neoplasm
Identification of one or more recurrent genetic abnormalities aids in prognostication and clinical management of patients with a myeloproliferative neoplasm
Specific mutations, cytogenetic abnormalities, or fusions has diagnostic or prognostic significance and inform clinical management

### Genetics

TABLE 11
Genes Tested for Mutations (single nucleotide
variants, insertions, deletions)

| | | | | |
|---|---|---|---|---|
| ASXL1 | IDH1 | KMT2C | SF3B1 | TP53 |
| CALR | IDH2 | MPL | SH2B3 | U2AF1 |
| CBL | IKZF1 | NFE2 | SRSF2 | |
| EZH2 | JAK2 | PPM1D | TET2 | |

TABLE 12
Genes Tested for Copy Number Variants

| | |
|---|---|
| ATM | RB1 |

TABLE 13
Fusion Genes Tested

FGFR1
PDGFRA
PDGFRB

### Results

### Genes Tested for Mutations

Positive: a somatic mutation in one of the tested genes was detected
Negative: no mutations were detected in the sequenced genes

### Genes Tested for Copy Number Variants

Positive: a copy number variant in one of the tested genes was detected
Negative: no copy number variants were detected in the sequenced genes

### Fusions Tested

Positive: a fusion involving one of the tested genes was detected
Negative: no fusions were detected in the sequenced genes

TABLE 14
Analytical Sensitivity

| | Variant Allelic | Analytical |
|---|---|---|
| Variant Class | Frequency | Sensitivity |
| Single nucleotide | 5% | 95% |
| variants | | |
| Insertions | 5% | 95% |
| deletions < 20 bp | | |
| Insertions | 5% | 95% |
| deletions => 20 bp | | |
| Fusions | 5% | 95% |

### Specimen Requirements

Peripheral blood: 5 mL in EDTA tube.
Bone marrow: 2 mL in EDTA tube.

TABLE 15
Applicable MPNs

| | |
|---|---|
| D45 | Polycythemia vera |
| D47.1 | Chronic myeloproliferative disease |
| D47.3 | Essential (hemorrhagic) thrombocythemia |

### Example 5: Non-Hodgkin Lymphoma Genetics Panel

Non-Hodgkin lymphomas (NHL) comprise a diverse group of hematologic malignancies of both B and T cell origin. The clinical presentation of NHL varies per subtype and can range from aggressive disease subtypes such as diffuse large B cell lymphoma (DLBCL), or Burkitt lymphoma (BL) to a much more indolent course as often seen with follicular lymphoma (FL) or marginal zone lymphoma (MZL). Despite their typical less acute behavior than acute myeloid leukemia, NHL can present as immediate life-threatening disease with associated complications such as spinal cord compression, lymphomatous meningitis and/or central nervous system mass lesions, airway obstruction, pericardial tamponade, superior vena cava obstruction, gastrointestinal obstruction or liver failure, or hydronephrosis or renal failure.

Many, but not all, subtypes of NHL exhibit one or more well-defined fusions that include DLBCL & FL: rearrangements of BCL6, BCL2, and MYC, MCL: rearrangement of CCND1, and MZL: rearrangement of MALT1.

While some well-defined recurrent genetic mutations in CLL are associated with an unfavorable prognosis, others occur as an escape mechanism to a particular therapy. Recurrent genetic mutations in NHL include;
TP53: NHL cells without normal TP53 function cannot trigger apoptosis in response to
BTK, PCLG2: Following treatment that targets the BCR pathway, mutations in BTK are assumed to be predictive of worse clinical outcomes with BCR inhibitors in MCL and MZL
BIRC3, TRAF2 (MCL): Mutations in BIRC3 and TRAF2 are associated with resistance to BCR inhibitors in MCL.
DNMT3A, TET2, IDH2: aid in the diagnosis of peripheral T-cell lymphoma (PTCL)
EZH2: EZH2 inhibitor tazemetostat for treatment of relapsed/refractory FL with EZH2 mutations
MYD88, CXCR4: mutations in MYD88 and CXCR4 predict aggressive behavior and response to BCR inhibitors in lymphoplasmacytic lymphoma and Waldenstrom macroglobulinemia

### Testing

Identification of one or more recurrent genetic abnormalities aids in prognostication and clinical management of patients with NHL
Initial management of CLL patients is determined by a combination of structural variants and specific mutations
Specific mutations are associated with resistance to targeted therapies.

### Genetics

TABLE 16
Genes Tested for Mutations (single nucleotide
variants, insertions, deletions)

| | | |
|---|---|---|
| BIRC3 | EZH2 | TET2 |
| BTK | IDH2 | TP53 |
| CXCR4 | MYD88 | TRAF2 |
| DNMT3A | PLCG2 | MAP2K1 |
| BRAF | MEK | |

TABLE 17
Fusion Genes Tested

| | |
|---|---|
| BCL6 | MALT1 |
| BCL2 | MYC |
| CCND1 | |

### Results

### Genes Tested for Mutations

Positive: a somatic mutation in one of the tested genes was detected
Negative: no mutations were detected in the sequenced genes

### Fusions

Positive: a fusion involving one of the tested genes was detected
Negative: no fusions were detected in the sequenced genes

TABLE 18
Analytical Sensitivity

| | Variant Allelic | Analytical |
|---|---|---|
| Variant Class | Frequency | Sensitivity |
| Single nucleotide | 5% | 95% |
| variants | | |
| Insertions | 5% | 95% |
| deletions < 20 bp | | |
| Insertions | 5% | 95% |
| deletions => 20 bp | | |

TABLE 19
Applicable NHLs

| | |
|---|---|
| C83.00 -hyphen C83.09 | Small cell B cell lymphoma [splenic marginal zone lymphoma] |
| C83.10 -hyphen C83.19 | Mantle cell lymphoma |
| C90.00 -hyphen C90.02 | Multiple myeloma |
| C83.30 -hyphen C83.39 | Diffuse large B-hyphencell lymphoma |
| C84.40 -hyphen C84.49 | Peripheral T-hyphencell lymphoma, not classified |
| D47.z1 | Post-hyphentransplant lymphoproliferative disorder (PTLD) |

### Example 6: Chronic Lymphocytic Leukemia Genetics Panel

Chronic lymphocytic leukemia/small lymphocytic lymphoma (CLL/SLL) is a mature B cell neoplasm characterized by a progressive accumulation of monoclonal B lymphocytes in the bone marrow, peripheral blood, and lymph nodes. CLL/SLL is the most common leukemia in adults in Western countries, accounting for approximately 25 to 35 percent of all leukemias in the United States.

Most cases of CLL exhibit at least one of four well-defined structural variants that include:
13q14 deletion: Deletions of part of the long arm of chromosome 13, more specifically 13q14 that includes the gene RB1, miR15A, and miR16A, are the most common molecular cytogenetic abnormality in CLL, and can be found in an estimated 50% of CLL. 13q14 deletion is considered a favorable prognostic marker.
Trisomy 12: Detected in 10-20% of CLL and is considered a favorable prognostic marker.
11q22-23 deletion: Deletions of 1 q22-23 involving the ATM (ataxia telangiectasia mutated), are detected in an estimated 20% of CLL. 11q22-23 deletion is an unfavorable prognostic factor and has been associated with rapidly progressive disease and inferior survival.
17p12-13 deletion: Deletions of the P53 tumor suppressor gene at 17p12-13 is the least common of these four well-defined structural variants seen in approximately 5-10% of CLL. 17p12-13 deletion is an unfavorable prognostic factor and has been associated with shorter survival times and resistance to treatment. 17p12-13 deletion and or TP53 mutational status combined with IGHV somatic hypermutation determines initial management of CLL.

Recurrent genetic mutations in CLL are less common than recurrent structural variants and typically are present in 5 to 10% of CLL. While some well-defined recurrent genetic mutations in CLL are associated with an unfavorable prognosis, others occur as an escape mechanism to a particular therapy. Recurrent genetic mutations in CLL include:
TP53: CLL cells without normal TP53 function cannot trigger apoptosis in response to chemotherapy-induced DNA damage. TP53 mutational status and or 17p12-13 deletion combined with IGHV somatic hypermutation determines initial management of CLL.
ATM: CLL cells without normal ATM function are unable to respond appropriately to chemotherapy-induced DNA damage.
NOTCHL: Activating mutations in NOTCH1 are associated with lack of response to rituximab, but benefit from a different anti-CD20 therapy Obinutuzumab.
SF3B1: Mutations in SF3B1 have been identified as an independent marker associated with worse PFS and OS following initial chemoimmunotherapy.
BTK: Following treatment that targets the BCR pathway, mutations in BTK are assumed to be predictive of worse clinical outcomes with BCR inhibitors.
BIRC3: similar to TP53 mutations and outcome as an independent risk factor for poor outcome
PCLG2: Following treatment that targets the BCR pathway, mutations in PLCg2 are assumed to be predictive of worse clinical outcomes with BCR inhibitors.
BCL2: Following treatment with venetoclax, mutations in BCL2 are assumed to be predictive of worse outcomes upon retreatment with venetoclax-based therapy.
CKDN2A/CKDN2B: associated with Richter's transformation

### Testing

Identification of one or more recurrent genetic abnormalities aids in prognostication and clinical management of patients with CLL
Initial management of CLL patients is determined by a combination of structural variants and specific mutations
Specific mutations are associated with resistance to targeted therapies.

### Genetics

TABLE 20
Genes Tested for Mutations (single nucleotide
variants, insertions, deletions)

| | | |
|---|---|---|
| ATM | NOTCH1 | TP53 |
| BCL2 | PLCG2 | |
| BTK | SF3B1 | |
| BIRC3 | CKDN2A/ | CKDN2B |

TABLE 21
Genes Tested for Copy Number Variants

ATM
RB1
TP53

### Results

### Genes Tested for Mutations

Positive: a somatic mutation in one of the tested genes was detected
Negative: no mutations were detected in the sequenced genes

### Genes Tested for Copy Number Variants

Positive: a copy number variant in one of the tested genes was detected
Negative: no copy number variants were detected in the sequenced genes

TABLE 22
Analytical Sensitivity

| | Variant Allelic | Analytical |
|---|---|---|
| Variant Class | Frequency | Sensitivity |
| Single nucleotide | 5% | 95% |
| variants | | |
| Insertions | 5% | 95% |
| deletions < 20 bp | | |
| Insertions | 5% | 95% |
| deletions => 20 bp | | |

TABLE 23
Applicable CLLs

| | |
|---|---|
| C91.10 -hyphen C91.12 | Chronic lymphocytic leukemia of B-cell type |

### Example 7: Multiple Myeloma Genetics Panel

Multiple myeloma (MM) is a neoplastic proliferation of plasma cells producing a monoclonal immunoglobulin characterized primarily involving the bone marrow. MM is an extremely heterogeneous disease with some patients having rapidly progressive symptoms and others have a more indolent clinical presentation. To predict the progression of MM a Revised International Staging System (R-ISS) based upon serum B2M, serum albumin, serum lactate dehydrogenase (LDH), and genetic changes is used to stratify patients into three risk groups. As a part of this analysis genetic changes are classified into high versus standard risk.

MM standard risk genetic changes include:
hyperdiploid karyotype characterized by trisomies of odd numbered chromosomes #3, #5, #7. #9, #11, #15, and #19
CCND1-IGH, t(l 1;14)(1q3;q32)t(6;14)

MM high risk genetic changes include;
CCND3-IGH t(6;14)(p21;q32)
FGFR3-IGH t(4;14)(p16;q32)
del(17p)
add(1q)
   Additional genetic changes in MM of clinical significance include:
del(1p) associated with poor outcomes
mutations in ATM, BRAF, CDKN2C, CHEK2, DIS3, KRAS NRAS, RB1, STAT3 or TP53 associated with poor outcomes
reduced sensitivity to bortezomib in NRAS mutant MM
mutations in EGR1, IRF4, and TRAF3 associated with favorable outcome

### Testing

Identification of one or more recurrent genetic abnormalities for risk stratification of patients with MM
Mutations in NRAS are associated with reduced sensitivity to bortezomib
Prognostic markers of poor outcome

### Genetics

TABLE 24
Genes Tested for Mutations (single nucleotide
variants, insertions, deletions)

ATM
BRAF
CDKN2C
CHEK2
DIS3
EGR1
IRF4
KRAS
NRAS
RB1
STAT3
TP53
TRAF3

TABLE 25
Genes Tested for Copy Number Variants

TP53

TABLE 26
Fusions Tested

CCND1
CCND3

### Results

### Genes Tested for Mutations

Positive: a somatic mutation in one of the tested genes was detected
Negative: no mutations were detected in the sequenced genes

### Genes Tested for Copy Number Variants

Positive: a copy number variant in one of the tested genes was detected
Negative: no copy number variants were detected in the sequenced genes

### Fusions Tested

Positive: a fusion involving one of the tested genes was detected
Negative: no fusions were detected in the sequenced genes

TABLE 27
Analytical Sensitivity

| | Variant Allelic | Analytical |
|---|---|---|
| Variant Class | Frequency | Sensitivity |
| Single nucleotide | 5% | 95% |
| variants | | |
| Insertions | 5% | 95% |
| deletions < 20 bp | | |
| Insertions | 5% | 95% |
| deletions => 20 bp | | |

### Specimen Requirements

Peripheral blood: 5 mL in EDTA tube.
Bone marrow: 2 mL in EDTA tube.

TABLE 28
Applicable MMs

| | |
|---|---|
| C90.00 -hyphen C90.02 | Multiple myeloma |

1. A dual DNA-based and RNA-based next generation sequencing (NGS) assay for detecting a hematological neoplasm comprising a DNA component comprising an oligonucleotide and a detection probe targeting DNA isolated from the hematological neoplasm and an RNA component comprising oligonucleotide and a detection probe targeting RNA isolated from the hematological neoplasm, and wherein the hematopoietic neoplasm is selected from acute myeloid leukemia (AML), myelodysplastic syndrome (MDS), myeloproliferative neoplasm (MPN), non-Hodgkin lymphoma (NHL), chronic lymphocytic leukemia (CLL), and multiple myeloma (MM).

2. The dual DNA-based and RNA-based NGS assay of claim 1, wherein the nucleic acid sequence targeted by the DNA component encodes BRAF, EZH2, KIT, MYD88, TP53, TET2, or a gene expressed in AML, MDS, MPN, NHL, CLL, or MM.

3. The dual DNA-based and RNA-based NGS assay of claim 1, wherein the nucleic acid sequence targeted by the DNA component encodes a heme-related gene selected from the group consisting of ABL1, ACD, AKT1, AKT2, AKT3, ALK, ANKRD26, ARAF, ARID1A, ARID1B, ARID2, ASXL1, ASXL2, ATM, ATRX, B2M, BCL10, BCL11A, BCL2, BCL6, BCL7A, BCOR, BCORL1, BIRC2, BIRC3, BLM, BRAF, BRCA, BRCA2, BRIP1, BTG1, BTG2, BTK, CALR, CARD11, CASP8, CBFB, CBL, CBLB, CBLC, CCND1, CCND2, CCND3, CCNE1, CCR4, CCR6, CCR7, CD22, CD274, CD28, CD38, CD58, CD70, CD79A, CD79B, CD83, CDC25C, CDK4, CDK6, CDK7, CDKN1B, CDKN2A, CDKN2B, CDKN2C, CEBPA, CHEK2, CIITA, CKS1B, CNOT3, CRBN, CREBBP, CSF1R, CSF3R, CTC1, CTCF, CTNNB1, CUL4A, CUL4B, CUX1, CXCR4, CYLD, DAPK1, DCK, DDX3X, DDX41, DHX15, DIS3, DKC1, DNMT3A, DTX1, EBF1, EFL1, EGFR, EGR1, EGR2, ELANE, EP300, EPCAM, ERBB2, ERBB3, ERCC4, ETNK1, ETV6, EZH2, FANCA, FANCB, FANCC, FANCD2, FANCE, FANCF, FANCG, FANCI, FANCL, FAS, FAT1, FAT3, FAT4, FBXO11, FBXW7, FGFR1, FGFR2, FGFR3, FLT3, FOXO1, FYN, G6PC3, GATA1, GATA2, GATA3, GFI1, GNA13, GNAS, GPR34, GRB2, GTF2I, H1-2, H1-3, H1-4, H1-5, H3C2, HAX1, HLA-A, HLA-B, HLA-C, HRAS, ID3, IDH1, IDH2, IDH3A, IFNGR2, IGF1R, IKBKB, IKZF1, IKZF3, IL2RG, IL6, IL6R, IL7R, INO80, IRF1, IRF4, IRF8, ITPKB, JAK1, JAK2, JAK3, KDM6A, KIT, KLF2, KLHL6, KMT2A, KMT2D, KRAS, LCK, LMO2, LTB, LUC7L2, MALT1, MAP2K1, MAP2K4, MAP3K1, MAP3K14, MCL1, MECOM, MED12, MEF2B, MET, MGA, MLH1, MPEG1, MPL, MSH2, MSH6, MTOR, MYB, MYC, MYD88, NBN, NF1, NF2, NFE2, NFKB1, NFKB2, NFKBIA, NFKBIE, NFKBIZ, NHP2, NOP10, NOTCH1, NOTCH2, NPM1, NR3C1, NRAS, NSD2, NTRK1, P2RY8, PALB2, PAX5, PDCD1LG2, PDGFRA, PDGFRB, PDS5B, PHF6, PIGA, PIK3CA, PIK3CB, PIK3CD, PIK3CG, PIK3R1, PIK3R2, PIM1, PIM2, PIM3, PLCG1, PLCG2, PMS2, POT1, POU2AF1, POU2F2, PPM1D, PRDM1, PRKCB, PRPF8, PSMA1, PSMB5, PSMD1, PSMG2, PTCH1, PTEN, PTPN1, PTPN11, PTPN2, PTPN6, PTPRC, PTPRD, RAD21, RAD51, RAD51C, RASA2, RB1, RBBP6, RBM8A, RCOR1, REL, RHOA, RIT1, ROCK1, ROS1, RPL11, RPL35A, RPL5, RPS10, RPS14, RPS15, RPS19, RPS24, RPS26, RPS7, RTEL1, RUNX1, SAMD9, SAMD9L, SBDS, SBF2, SETBP1, SETD2, SETDB1, SF1, SF3B1, SGK1, SH2B3, SLC29A1, SLX4, SMARCA4, SMARCB1, SMC1A, SMC3, SOCS1, SOS1, SPEN, SPIB, SRC, SRP72, SRSF2, STAG2, STAT3, STAT5B, STAT6, STK11, SUZ12, SYK, TBL1XR1, TCF3, TENT5C, TERT, TET2, TGFBR2, THRAP3, TINF2, TLR2, TLR4, TMEM30A, TMSB4X, TNF, TNFAIP2, TNFAIP3, TNFRSF14, TP53, TRAF2, TRAF3, U2AF1, U2AF2, UBE2A, UBE2T, UBR5, USB1, VAV1, VPS45, WAS, WRAP53, WT1, XBP1, XPO1, ZFP36L1, and ZRSR2.

4. The dual DNA-based and RNA-based NGS assay of claim 1, wherein the DNA component of the NGS assay detects a variant selected from a group consisting of a single nucleotide variant, an insertion deletion (indel), a chimeric fusion, a structural variant, a copy number variant, and a chromosomal abnormality.

5. The dual DNA-based and RNA-based NGS assay of claim 1, wherein the DNA component of the NGS assay simultaneously detects more than one variant within the hematological neoplasm.

6. The dual DNA-based and RNA-based NGS assay of claim 1, wherein the gene expressed in AML is selected from ABL1, ASXL1, ASXL2, ATM, BCOR, BCR, CBFB, CREBBP, CEBPA, DEK, DNMT3A, EZH2, FGFR1, FLT3, IDH1, IDH2, JAK2, KIT, KMT2A, KRAS, MLF1, MKL1, MYH11, MECOM, NPM1, NRAS, NUP214, PDGFRA, PDGFRB, PHF6, PML, RBM15, RARA, RUNX1, RUNX1T1, SF3B1, SRSF2, STAG2, TP53, U2AF1, WT1, and ZRSR2.

7. The dual DNA-based and RNA-based NGS assay of claim 1, wherein the gene expressed in MDS is selected from ASXL1, ATM, BCOR, CALR, CBFB, CBL, CREBBP, DEK, DDX41, DNMT3A, ETV6, EZH2, FLT3, GATA2, IDH1, IDH2, JAK2, KMT2A, MECOM, MPL, MYH11, NPM1, NRAS, NUP214, PHF6, PML, PPM1D, PRDM16, RARA, RB1, RUNX1, RUNX1T1, SETBP1, SF3B1, STAG2, STAT3, SRSF2, TET2, TP53, U2AF1, WT1, and ZRSR2.

8. The dual DNA-based and RNA-based NGS assay of claim 1, wherein the gene expressed in MPN is selected from ASXL1, CBL, CALR, EZH2, IDH1, IDH2, IKZF1, JAK2, KMT2C, MPL, NFE2, PPM1D, SF3B1, SH2B3, SRSF2, TET2, TP53, and U2AF1.

9. The dual DNA-based and RNA-based NGS assay of claim 1, wherein the gene expressed in NHL is selected from BIRC3, BRAF, BTK, CXCR4, DNMT3A, EZH2, IDH2, MAP2K1, MEK, MYD88, PLCG2, TET2, TP53, and TRAF2.

10. The dual DNA-based and RNA-based NGS assay of claim 1, wherein the gene expressed in CLL is selected from ATM, BCL2, BTK, NOTCH1, PLCG2, RB1, SF3B1, and TP53.

11. The dual DNA-based and RNA-based NGS assay of claim 1, wherein the gene expressed in MM is selected from ATM, BRAF, CCND1, CCND3, CDKN2A, CDKN2C, CHEK2, DIS3, EGR1, IRF4, KRAS, NRAS, RB1, STAT3, TRAF3, and TP53.

12. The dual DNA-based and RNA-based NGS assay of claim 1, wherein the DNA component of the NGS assay detects one or more single nucleotide variations listed in Table 29.

13. The dual DNA-based and RNA-based NGS assay of claim 1, wherein the DNA component of the NGS assay simultaneously detects a gene variant and the cytogenic alternation selected from the group consisting of an EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, an odd number of chromosomes, and trisomy 21.

14. The dual DNA-based and RNA-based NGS assay of claim 1, wherein the gene variant and chromosomal abnormality detected in CCL comprise EGR1 variant and deletion of chromosome segment 5q31, EGR1 variant and deletion of chromosome 5, EGR1 variant and deletion of chromosome segment 5q, CSF1R variant and deletion of chromosome segments 5q33-5q34, ATM variant and deletion of chromosome segment 11q23, TP53 variant and deletion of chromosome segment of 17p13, TP53 variant and deletion of chromosome 17, CDKN2A variant and deletion of chromosome segment 9p21, CDKN2A variant and deletion of chromosome 9, PTEN variant and deletion of chromosome segment 10q23, RB1 variant and deletion of chromosome segment 13q14, POT1 variant and deletion of chromosome segment 7q31, duplication of chromosome 12, an odd number of chromosomes, or trisomy 21.

15. The dual DNA-based and RNA-based NGS assay of claim 1, wherein the RNA component of the NGS assay detects a chimeric gene fusion without prior knowledge of the fusion partners or the breakpoint translocation.

16. The dual DNA-based and RNA-based NGS assay of claim 1, wherein the fusion comprises a fusion of any two genes selected from the group comprising ABL1, AFF1, BCL11B, BCL6, BCR, CBFA2T3, CBFB, CCND3, CDK6, CEP43, CHD1, CHIC2, CITA, CREBBP, DAZAP1, DDX6, DEK, DUSP22, EBF1, EIF4A1, EPOR, EP300, ERG, ETV6, FGFR1, FGFR3, FLT3, FOXP1, FUS, GATA2, H2AX, IGH, IGK, IGL, IKZF1, IKZF2, IKZF3, JAK2, KANSL1, KAT6A, KLF2, KMT2A, MECOM, MKL1, MLF1, MLLT4, MLLT10, MN1, MPO, MYB, MYC, MYH11, MYH9, NF1, NOTCH1, NPM1, NTRK3, NUP98, P2RY8, PAG1, PAX5, PML, PBX1, PDCD1LG2, PICALM, PTK2B, RARA, RARG, RBM15, ROS1, RUNX1, SSBP2, SEMA6A, SETD2, STIL, TAL1, TBL1XR1, TCF3, TFG, TPM4, TRA, TRB, TRD, UBA2, VAV1, XPO1, ZFP36L2, ZNF362, ZNF384, DSCAM, NUTM1, BCL2, PDGFRB, TYK2, ALK, CCND1, TPM3, NIPBL, STAG2, MALT1, ETS2, DDX10, PDGFRA, MSI2, PRDM16, NFKB2, CRLF2, LMO2, CD28, NUP214, ZCCHC7, HOXA11, IRF4, GLIS2, AHI1, ACTB, RET, BIRC3, HOXA9, LYN, ATF7IP, STAT6, BMI1, KDM5A, SYK, RB1, MTCP1, SEC31A, NSD1, LMO1, NFIA, CTCF, RUNX1T1, FOXR1, ABL2, PRRC2B, YPEL5, TP73, MRTFA, CSF1R, PICALM, FNBP1, CUX1, SEPTIN9, TP63, SPI1, RNF213, TCL1A, ELL, DDX3X, or HNRNPC

17-29. (canceled)

30. The dual DNA-based and RNA-based NGS assay of claim 1, wherein the sequencing assay further comprises a gene library construction reagent.

31. The dual DNA-based and RNA-based NGS assay of claim 1, wherein the sequencing assay further comprises a gene amplification reagent.

32-34. (canceled)

35. A method of identifying or profiling a hematological neoplasm in a subject in need thereof, the method comprising
obtaining a tissue sample from the subject,
performing dual DNA-based and RNA-based next generation sequencing (NGS) assay on the tissue sample using an oligonucleotide and a detection probe targeting DNA isolated from the tissue sample and an oligonucleotide and a detection probe targeting RNA isolated from the tissue sample, and
detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM).

36-58. (canceled)

59. A method of treating a hematological neoplasm in a subject in need thereof, the method comprising
obtaining a tissue sample from the subject,
performing dual DNA-based and RNA-based next generation sequencing (NGS) assay on the tissue sample using an oligonucleotide and a detection probe targeting DNA isolated from the tissue sample and an oligonucleotide and a detection probe targeting RNA isolated from the tissue sample;
detecting a variant in the nucleic acid sequence, wherein the variant identifies or profiles the hematopoietic neoplasm selected from acute myeloid leukemias (AML), myelodysplastic syndromes (MDS), myeloproliferative neoplasms (MPN), non-Hodgkin lymphoma (NHL), chronic lymphocytic leukemia (CLL), and multiple myeloma (MM); and
administering to the subject with a therapeutically effective amount of an anti-cancer agent appropriate for the treatment of the hematological neoplasm identified.

60-81. (canceled)
